(19) United States
(12) Patent Application Publication
(10) Pub. No.: US 2024/0025907 A1
(43) Pub. Date: Jan. 25, 2024

(54) QUINAZOLINE PAN-KRas INHIBITORS

(71) Applicant: Mirati Therapeutics, Inc., San Diego, CA (US)

(72) Inventors: Xiaolun Wang, San Diego, CA (US); Anthony Ivetac, San Diego, CA (US); Svitlana Kulyk, San Diego, CA (US); John David Lawson, San Diego, CA (US); Matthew Arnold Marx, San Diego, CA (US); Christopher Ronald Smith, San Diego, CA (US)

(21) Appl. No.: 18/229,662

(22) Filed: Aug. 2, 2023

Related U.S. Application Data
(63) Continuation-in-part of application No. PCT/US23/12299, filed on Feb. 3, 2023.
(60) Provisional application No. 63/306,271, filed on Feb. 3, 2022.
(60) Provisional application No. 63/327,625, filed on Apr. 5, 2022.
(60) Provisional application No. 63/352,180, filed on Jun. 14, 2022.
(60) Provisional application No. 63/432,243, filed on Dec. 13, 2022.
(60) Provisional application No. 63/434,327, filed on Dec. 21, 2022.
(60) Provisional application No. 63/442,648, filed on Feb. 1, 2023.

(51) Int. Cl. C07D 487/04; C07D 451/00; C07D 519/00; C07D 401/04; C07D 471/10; C07D 409/14; A61P 35/00

(52) U.S. Cl. CPC C07D 487/04; C07D 451/00; C07D 519/00; C07D 401/04; C07D 471/10; C07D 409/14; A61P 35/00

(57) ABSTRACT

The present invention relates to compounds that inhibit at least one of KRas G12A, KRas G12C, KRas G12D, KRas G12R, KRas G12S, KRas G12V, KRas G13D and KRas Q61H, pharmaceutical compositions comprising the compounds and methods of use therefor.

### FIELD OF THE INVENTION

The present invention relates to compounds that inhibit multiple mutated forms of KRas, i.e., pan-KRas inhibitors. In particular, the present invention relates to pan-KRas compounds, pharmaceutical compositions comprising the compounds and methods of use therefor.

### BACKGROUND OF THE INVENTION

Kirsten Rat Sarcoma 2 Viral Oncogene Homolog (“KRas”) is a small GTPase and a member of the Ras family of oncogenes. KRas serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin Pharmcol. 13:394-401).

The role of activated KRas in malignancy was observed over thirty years ago (e.g., see Santos et al., (1984) Science 223:661-664). Aberrant expression of KRas accounts for up to 20% of all cancers and oncogenic KRas mutations that stabilize GTP binding and lead to constitutive activation of KRas. KRas mutations at codons 12, 13, 61 and other positions of the KRas primary amino acid sequence are present in 88% of all pancreatic adenocarcinoma patients, 50% of all colon/rectal adenocarcinoma patients, and 32% lung adenocarcinoma patients (e.g., see Prior et all., (2020) Cancer Res 80:2969-74). A recent publication also suggested wild type Kras inhibition could be a viable therapeutic strategy to treat KRas^{WT} dependent cancers (e.g., see Bery et al., (2020) Nat. Commun. 11: 3233).

The well-known role of KRas in malignancy and the discovery of these frequent mutations in KRas in various tumor types made KRas a highly attractive target of the pharmaceutical industry for cancer therapy. Notwithstanding thirty years of large-scale discovery efforts to develop inhibitors of KRas for treating cancer, no KRas inhibitor has yet demonstrated sufficient safety and/or efficacy to obtain regulatory approval (e.g., see McCormick (2015) Clin Cancer Res. 21 (8):1797-1801).

Compounds that inhibit KRas activity are still highly desirable and under investigation, including those that disrupt effectors such as guanine nucleotide exchange factors (e.g., see Sun et al., (2012) Agnew Chem Int Ed Engl. 51(25):6140-6143 doi: 10.1002/anie201201358) as well recent advances in the covalent targeting of an allosteric pocket of KRas G12C (e.g., see Ostrem et al., (2013) Nature 503:548-551 and Fell et al., (2018) ACS Med. Chem. Lett. 9:1230-1234). Clearly there remains a continued interest and effort to develop inhibitors of KRas, particularly inhibitors of activating KRas mutants.

Thus, there is a need to develop new pan-KRas inhibitors that demonstrate sufficient efficacy for treating KRas-mediated cancers.

### SUMMARY OF THE INVENTION

In one aspect of the invention there are provided compound of Formula (I):

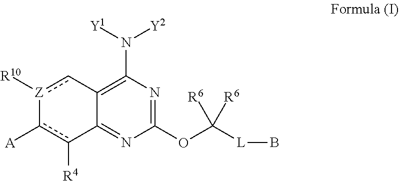

or a pharmaceutically acceptable salt thereof, wherein:

A is aryl or heteroaryl, wherein the aryl or the heteroaryl is optionally substituted with 1-4 R¹;

B is:

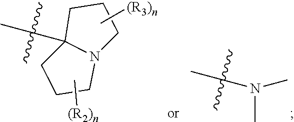

Y¹ is hydrogen, hydroxy, halogen, L-SO₂—NH₂, L-OH, C1-C4 alkyl, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle optionally substituted with 1-2 oxo (═O) or oxo-containing substituent, and optionally further substituted with 1-2 R⁸;

Y² is hydrogen or C1-C4 alkyl;

or Y¹ and Y² join to form:

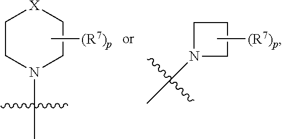

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—;

each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxy C1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;

each R² is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —OC(O)N(R⁵)₂, —CO₂R⁵, or —CO₂N(R⁵)₂;

each R³ is independently hydrogen, deuterium hydroxy, halogen, C1-C3 alkyl, ═CH₂, ═CH(halogen), ═C(halogen)₂, C1-C3 cyanoalkyl, C1-C3 hydroxyalkyl, HC(═O)—, —COC(O)N(R⁵)₂, —CO₂R⁵, or —CO₂N(R⁵)₂;

R⁴ is hydrogen, halogen or C1-C3 alkyl;

each R⁵ is independently hydrogen or C1-C3 alkyl;

each R⁶ is independently hydrogen, hydroxy, C1-C4 hydroxyalkyl or heteroaryl;

each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, aryl, —CH₂—S(O)₂NH₂, or heteroaryl optionally independently substituted with 1-2 C1-C3 alkyl, —CN or C(O)NH₂,

two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),

two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and

two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;

each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;

each R⁹ is independently C1-C3 alkyl, hydroxy, halogen, oxo (═O), —O—(C1-C3 alkyl), —(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;

R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;

L is a bond, —C1-C4 alkyl-, —NH—, —N(C1-C3 alkyl)- or cyclopropyl-CH₂—;

Z is C or O, wherein if Z is C the 6-membered ring that includes Z is aromatic, and wherein if Z is O the 6-membered ring that includes Z is an oxane;

each n is 0-3;

o is 1-6; and

p is 1-8.

In another aspect of the invention, pharmaceutical compositions are provided comprising a therapeutically effective amount of a compound of the present invention or a pharmaceutically acceptable salt thereof and a pharmaceutically acceptable excipient.

In yet another aspect of the invention, methods for inhibiting the activity of cells containing wild type KRas or one or more Kras mutations, for instance the Kras mutations G12A, G12C, G12D, G12R, G12S, G12V, G13D and/or Q61H, in a in a cell, comprising contacting the cell with a compound of Formula (I), or a pharmaceutically acceptable salt thereof, or a pharmaceutical composition thereof as defined herein. In one embodiment, the contacting is in vitro. In one embodiment, the contacting is in vivo.

Also provided herein is a method of inhibiting cell proliferation, in vitro or in vivo, the method comprising contacting a cell with an effective amount of a compound of Formula (I), or a pharmaceutically acceptable salt thereof, or a pharmaceutical composition thereof as defined herein.

Also provided are methods for treating cancer in a patient comprising administering a therapeutically effective amount of a compound or pharmaceutical composition of the present invention or a pharmaceutically acceptable salt thereof to a patient in need thereof.

Also provided herein is a method of treating a Kras wild type, Kras G12A, G12C, G12D, G12R, G12S, G12V, G13D and/or Q61H-associated disease or disorder in a patient in need of such treatment, the method comprising administering to the patient a therapeutically effective amount of a compound of Formula (I), or a pharmaceutically acceptable salt thereof, or a pharmaceutical composition thereof as defined herein.

Also provided herein is a compound of Formula (I), or a pharmaceutically acceptable salt thereof, or a pharmaceutical composition thereof as defined herein for use in therapy.

Also provided herein is a compound of Formula (I), or a pharmaceutically acceptable salt thereof or a pharmaceutical composition thereof as defined herein for use in the treatment of cancer.

Also provided herein is a compound of Formula (I), or a pharmaceutically acceptable salt thereof for use in the inhibition of Kras wild type or multiple types of Kras mutations, for instance Kras G12A, G12C, G12D, G12R, G12S, G12V, G13D and/or Q61H mutations.

Also provided herein is a compound of Formula (I), or a pharmaceutically acceptable salt thereof or a pharmaceutical composition thereof as defined herein, for use in the treatment of a Kras wild type associated disease or disorder or a Kras mutation G12A, G12C, G12D, G12R, G12S, G12V, G13D and/or Q61H-associated disease or disorder.

Also provided herein is the use of a compound of Formula (I), or a pharmaceutically acceptable salt thereof, as defined herein in the manufacture of a medicament for the treatment of cancer.

Also provided herein is a use of a compound of Formula (I), or a pharmaceutically acceptable salt thereof, as defined herein in the manufacture of a medicament for the inhibition of activity of the wild type form of Kras or mutated forms of Kras, including the mutations: G12A, G12C, G12D, G12R, G12S, G12V, G13D and/or Q61H.

Also provided herein is the use of a compound of Formula (I), or a pharmaceutically acceptable salt thereof, as defined herein, in the manufacture of a medicament for the treatment of a Kras wild type associated disease or disorder or a Kras G12A, G12C, G12D, G12R, G12S, G12V, G13D and/or Q61H-associated disease or disorder.

Also provided herein is a method for treating cancer in a patient in need thereof, the method comprising (a) determining that the cancer is associated with Kras wild type or a Kras G12A, G12C, G12D, G12R, G12S, G12V, G13D and/or Q61H mutation (i.e., a Kras G12A, G12C, G12D, G12R, G12S, G12V, G13D and/or Q61H-associated cancer); and (b) administering to the patient a therapeutically effective amount of a compound of Formula (I), or a pharmaceutically acceptable salt thereof, or a pharmaceutical composition thereof.

One potential utility of the herein-described pan-Kras inhibitors, including pan-Kras inhibitors such as 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide (Example 23 herein), is for the treatment of cancers that develop resistance following long-term treatment with Kras G12C inhibitors. Thus, embodiments of the invention include those wherein a patient suffering from cancer is treated with a herein-described pan-Kras inhibitor such as Example 5 after treatment with a G12C inhibitor becomes ineffective or less effective due to the emergence of resistance-imparting mutations.

Treatment of Kras G12C mutant cancers with covalent Kras G12C inhibitors such as adagrasib (MRTX849) or sotorasib (AMG510) may result in the incorporation of additional mutations that confer resistance to adagrasib. These mutations could confer resistance through numerous mechanisms.

Mutations that change the mutant cysteine at codon 12 to another amino acid would render the current covalent Kras G12C inhibitors ineffective since current inhibitors make a covalent bond with the mutant cysteine amino acid side chain. Likewise, in patients that have one wild type Kras allele in addition to the Kras G12C-mutant allele, mutations in the wild type codon 12 glycine to another codon would allow bypass signaling in these tumors through the novel mutant protein. The repertoire of codon 12 mutations that can occur with a single nucleotide substitution in the wild type gene (glycine codon) includes mutations commonly observed in cancer such as G12S, G12V, G12R, G12C. The repertoire of codon 12 mutations that can occur with single nucleotide base substitutions of the cysteine codon 12 include mutations not frequently observed in cancer, G12Y, G12F and G12W, in addition to G12S and G12R.

Second-site mutations may also occur in another location in the Kras G12C mutant gene that confers resistance to Kras G12C inhibitor treatment. These mutations may confer resistance through different mechanisms. RAS proteins are small GTPases that normally cycle between an active, GTP-bound state and an inactive, GDP-bound state. RAS proteins are loaded with GTP through guanine nucleotide exchange factors (GEFs; e.g., SOS1) which are activated by upstream receptor tyrosine kinases, triggering subsequent interaction with effector proteins that activate RAS-dependent signaling. RAS proteins hydrolyze GTP to GDP through their intrinsic GTPase activity which is dramatically enhanced by GTPase-activating proteins (GAPs). Mutations at codons 12 and 13 in RAS proteins impair GAP-stimulated GTP hydrolysis leaving RAS predominantly in the GTP-bound, active state. Covalent Kras G12C inhibitors in current clinical development only bind GDP-bound Kras G12C. Mutations such as Q61 codon mutations, which may or may not occur on the same allele as the G12C mutation, reduce the intrinsic GTPase activity of Kras and may represent a mechanism of resistance to Kras G12C inhibitor treatment by shifting Kras into the GTP-loaded state where it is not susceptible to covalent inhibition. Co-mutations such as R68, H95 and Y96 may be present along with the Kras G12C mutation and may diminish the binding affinity of Kras G12C inhibitors to the Switch II binding pocket.

The herein-described pan-Kras inhibitors may demonstrate activity against common as well as uncommon codon 12 mutations or mutations that occur in the Kras protein that diminish binding of Kras G12C inhibitors to the Kras protein.

Also provided herein is a process for preparing a compound of Formula (I), or a pharmaceutically acceptable salt thereof.

Also provided herein is a compound of Formula (I), or a pharmaceutically acceptable salt thereof obtained by a process of preparing the compound as defined herein.

### DETAILED DESCRIPTION OF THE INVENTION

The present invention relates to inhibitors of Kras wild type and/or multiple mutated forms of Kras, for instance Kras G12A, G12C, G12D, G12R, G12S, G12V, G13D and/or Q61H mutations. In particular, the present invention relates to compounds that inhibit the activity of Kras wild type and/or Kras mutations such as G12A, G12C, G12D, G12R, G12S, G12V, G13D and/or Q61H, pharmaceutical compositions comprising a therapeutically effective amount of the compounds and methods of use therefor.

### Definitions

Unless defined otherwise, all technical and scientific terms used herein have the same meaning as is commonly understood by one of skill in the art to which this invention belongs. All patents, patent applications, and publications referred to herein are incorporated by reference.

Compounds described herein comprise atoms in both their natural isotopic abundance and in non-natural abundance. The disclosed compounds can be isotopically-labelled or isotopically-substituted compounds identical to those described, but for the fact that one or more atoms are replaced by an atom having an atomic mass or mass number different from the atomic mass or mass number typically found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F and ³⁶Cl, respectively. Compounds further comprise prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of the present invention and prodrugs thereof can generally be prepared by carrying out the procedures below, by substituting a readily available isotopically labelled reagent for a non-isotopically labelled reagent.

As used herein, “wild type Kras” refers to a non-mutant form of a mammalian Kras protein. The assignment of amino acid codon and residue positions for human Kras is based on the amino acid sequence identified by UniProtKB/Swiss-Prot P01116: Variantp.Gly12Asp. As used herein, a “wild type Kras inhibitor” refers to compounds of the present invention that are represented by Formula (I), as described herein. These compounds are capable of negatively modulating or inhibiting all or a portion of the enzymatic activity of wild type Kras G12A. A “wild type Kras-associated disease or disorder” as used herein refers to diseases or disorders associated with or mediated by or having wild type Kras. A non-limiting example of a wild type Kras-associated disease or disorder is a wild type Kras-associated cancer.

As used herein, “Kras G12A” refers to a mutant form of a mammalian Kras protein that contains an amino acid substitution of an alanine for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12A inhibitor” refers to compounds of the present invention that are represented by Formula (I), as described herein. These compounds are capable of negatively modulating or inhibiting all or a portion of the enzymatic activity of Kras G12A. A “Kras G12A-associated disease or disorder” as used herein refers to diseases or disorders associated with or mediated by or having a Kras G12A mutation. A non-limiting example of a Kras G12A-associated disease or disorder is a Kras G12A-associated cancer.

As used herein, “Kras G12C” refers to a mutant form of a mammalian Kras protein that contains an amino acid substitution of a cysteine for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12C inhibitor” refers to compounds of the present invention that are represented by Formula (I), as described herein. These compounds are capable of negatively modulating or inhibiting all or a portion of the enzymatic activity of Kras G12C. A “Kras G12C-associated disease or disorder” as used herein refers to diseases or disorders associated with or mediated by or having a Kras G12C mutation. A non-limiting example of a Kras G12C-associated disease or disorder is a Kras G12CD-associated cancer.

As used herein, “Kras G12D” refers to a mutant form of a mammalian Kras protein that contains an amino acid substitution of an aspartic acid for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12D inhibitor” refers to compounds of the present invention that are represented by Formula (I), as described herein. These compounds are capable of negatively modulating or inhibiting all or a portion of the enzymatic activity of Kras G12D. A “Kras G12D-associated disease or disorder” as used herein refers to diseases or disorders associated with or mediated by or having a Kras G12D mutation. A non-limiting example of a Kras G12D-associated disease or disorder is a Kras G12D-associated cancer.

As used herein, “Kras G12R” refers to a mutant form of a mammalian Kras protein that contains an amino acid substitution of an arginine for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12R inhibitor” refers to compounds of the present invention that are represented by Formula (I), as described herein. These compounds are capable of negatively modulating or inhibiting all or a portion of the enzymatic activity of Kras G12R. A “Kras G12R-associated disease or disorder” as used herein refers to diseases or disorders associated with or mediated by or having a Kras G12R mutation. A non-limiting example of a Kras G12R-associated disease or disorder is a Kras G12R-associated cancer.

As used herein, “Kras G12S” refers to a mutant form of a mammalian Kras protein that contains an amino acid substitution of a serine for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12S inhibitor” refers to compounds of the present invention that are represented by Formula (I), as described herein. These compounds are capable of negatively modulating or inhibiting all or a portion of the enzymatic activity of Kras G12S. A “Kras G12S-associated disease or disorder” as used herein refers to diseases or disorders associated with or mediated by or having a Kras G12S mutation. A non-limiting example of a Kras G12S-associated disease or disorder is a Kras G12S-associated cancer.

As used herein, “Kras G12V” refers to a mutant form of a mammalian Kras protein that contains an amino acid substitution of a valine for a glycine at amino acid position 12. The assignment of amino acid codon and residue positions for human Kras is based on the amino acid sequence identified by UniProtKB/Swiss-Prot P01116: Variantp.Gly12Asp. As used herein, a “Kras G12V inhibitor” refers to compounds of the present invention that are represented by Formula (I), as described herein. These compounds are capable of negatively modulating or inhibiting all or a portion of the enzymatic activity of Kras G12V. A “Kras G12V-associated disease or disorder” as used herein refers to diseases or disorders associated with or mediated by or having a Kras G12V mutation. A non-limiting example of a Kras G12V-associated disease or disorder is a Kras G12V-associated cancer.

As used herein, “Kras G13D” refers to a mutant form of a mammalian Kras protein that contains an amino acid substitution of an aspartic acid for a glycine at amino acid position 13. The assignment of amino acid codon and residue positions for human Kras is based on the amino acid sequence identified by UniProtKB/Swiss-Prot P01116: Variantp.Gly12Asp. As used herein, a “Kras G13D inhibitor” refers to compounds of the present invention that are represented by Formula (I), as described herein. These compounds are capable of negatively modulating or inhibiting all or a portion of the enzymatic activity of Kras G13D. A “Kras G13D-associated disease or disorder” as used herein refers to diseases or disorders associated with or mediated by or having a Kras G13D mutation. A non-limiting example of a Kras G13D-associated disease or disorder is a Kras G13D-associated cancer.

As used herein, “Kras Q61H” refers to a mutant form of a mammalian Kras protein that contains an amino acid substitution of a histidine for a glutamine at amino acid position 61. The assignment of amino acid codon and residue positions for human Kras is based on the amino acid sequence identified by UniProtKB/Swiss-Prot P01116: Variantp.Gly12Asp. As used herein, a “Kras Q61H inhibitor” refers to compounds of the present invention that are represented by Formula (I), as described herein. These compounds are capable of negatively modulating or inhibiting all or a portion of the enzymatic activity of Kras Q61H. A “Kras Q61H-associated disease or disorder” as used herein refers to diseases or disorders associated with or mediated by or having a Kras Q61H mutation. A non-limiting example of a Kras Q61H-associated disease or disorder is a Kras Q61H-associated cancer.

As used herein, the term “subject,” “individual,” or “patient,” used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the subject has been identified or diagnosed as having a cancer having wild type Kras or a Kras G12A, G12C, G12D, G12R, G12S, G12V, G13D and/or Q61H mutation (e.g., as determined using a regulatory agency-approved, e.g., FDA-approved, assay or kit). In some embodiments, the subject has a tumor that is positive for wild type Kras or a Kras G12A, G12C, G12D, G12R, G12S, G12V, G13D and/or Q61H mutation (e.g., as determined using a regulatory agency-approved assay or kit). The subject can be a subject with a tumor(s) that is positive for wild type Kras or a Kras G12A, G12C, G12D, G12R, G12S, G12V, G13D and/or Q61H mutation (e.g., identified as positive using a regulatory agency-approved, e.g., FDA-approved, assay or kit). The subject can be a subject whose tumors have wild type Kras or a Kras G12A, G12C, G12D, G12R, G12S, G12V, G13D and/or Q61H mutation (e.g., where the tumor is identified as such using a regulatory agency-approved, e.g., FDA-approved, kit or assay). In some embodiments, the subject is suspected of having wild type Kras or a Kras G12A, Kras G12C, Kras G12D, Kras G12R, Kras G12S, Kras G12V, Kras G13D or Kras Q61H gene-associated cancer. In some embodiments, the subject has a clinical record indicating that the subject has a tumor that has wild type Kras or a Kras G12A, G12C, G12D, G12R, G12S, G12V, G13D and/or Q61H mutation (and optionally the clinical record indicates that the subject should be treated with any of the compositions provided herein).

In some embodiments of any of the methods or uses described herein, an assay is used to determine whether the patient has wild type Kras or a Kras G12A, G12C, G12D, G12R, G12S, G12V, G13D and/or Q61H mutation using a sample (e.g., a biological sample or a biopsy sample (e.g., a paraffin-embedded biopsy sample) from a patient (e.g., a patient suspected of having wild type Kras-associated or a Kras G12A, G12C, G12D, G12R, G12S, G12V, G13D and/or Q61H-associated cancer, a patient having one or more symptoms of wild type Kras-associated or a Kras G12A, G12C, G12D, G12R, G12S, G12V, G13D and/or Q61H-associated cancer, and/or a patient that has an increased risk of developing wild type Kras-associated or a Kras G12A, G12C, G12D, G12R, G12S, G12V, G13D and/or Q61H-associated cancer) can include, for example, next generation sequencing, immunohistochemistry, fluorescence microscopy, break apart FISH analysis, Southern blotting, Western blotting, FACS analysis, Northern blotting, and PCR-based amplification (e.g., RT-PCR and quantitative real-time RT-PCR). As is well-known in the art, the assays are typically performed, e.g., with at least one labelled nucleic acid probe or at least one labelled antibody or antigen-binding fragment thereof.

The term “regulatory agency” is a country's agency for the approval of the medical use of pharmaceutical agents with the country. For example, a non-limiting example of a regulatory agency is the U.S. Food and Drug Administration (FDA).

The term “acyl” refers to —C(O)CH₃.

The terms “C1-C6 alkyl”, “C1-C4 alkyl” and “C1-C3 alkyl” as employed herein refers to straight and branched chain aliphatic groups having from 1-6 carbon atoms, or 1-4 carbon atoms, or 1-3 carbon atoms, respectively. Examples of alkyl groups include, without limitation, methyl, ethyl, propyl, isopropyl, butyl, isobutyl, sec-butyl, tert-butyl, pentyl, and hexyl.

The terms “C1-C3 haloalkyl” and “C1-C4 haloalkyl” refer to a C1-C3 alkyl chain or C1-C4 alkyl chain, respectively, as defined herein in which one or more hydrogen has been replaced by a halogen. Examples include trifluoromethyl, difluoromethyl and fluoromethyl.

An “C1-C4 alkylene,” group is a C1-C4 alkyl group, as defined hereinabove, that is positioned between and serves to connect two other chemical groups. Exemplary alkylene groups include, without limitation, methylene, ethylene, propylene, and butylene.

The terms “C1-C3 alkoxy” and “C1-C4 alkoxy” refer to —OC1-C3 alkyl and —OC1-C4 alkyl, respectively, wherein the alkyl portion is as defined herein above.

The term “cycloalkyl” as employed herein includes saturated and partially unsaturated cyclic hydrocarbon groups having 3 to 12 carbons, for example 3 to 8 carbons, and as a further example 3 to 6 carbons, wherein the cycloalkyl group additionally is optionally substituted with one or more R⁸ or R⁹ groups as defined herein. Examples of cycloalkyl groups include, without limitation, cyclopropyl, cyclobutyl, cyclopentyl, cyclopentenyl, cyclohexyl, cyclohexenyl, cycloheptyl, and cyclooctyl. The term “cycloalkyl” also includes bridged cycloalkyls, such as bicyclo[1.1.1]pentanyl.

As used herein, the terms “C1-C3 hydroxyalkyl” and “C1-C4 hydroxyalkyl” refer to —C1-C3 alkylene-OH and —C1-C4 alkylene-OH, respectively.

As used herein, the term “C2-C4 hydroxyalkynyl” refers to —C2-C4 alkynylene-OH.

An “aryl” group is a C6-C14 aromatic moiety comprising one to three aromatic rings, which is optionally substituted with one or more R⁸ or R⁹ groups as defined herein. As one embodiment, the aryl group is a C₆-C₁₀ aryl group. Examples of aryl groups include, without limitation, phenyl, naphthyl, anthracenyl, fluorenyl, and dihydrobenzofuranyl. “Aryl” also refers to bicyclic or tricyclic ring systems in which one or two rings, respectively, of said aryl ring system may be saturated or partially saturated, and wherein if said ring system includes two saturated rings, said saturated rings may be fused or spirocyclic. An example of an aryl ring system comprising two saturated rings wherein the rings are spirocyclic includes the following ring system:

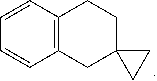 [SMILES: c1ccc2c(c1)CCC1(CC1)C2]

An “araC1-C6 alkyl” or “arylalkyl” group comprises an aryl group covalently linked to an alkyl group, either of which may independently be optionally substituted or unsubstituted. An example of an aralkyl group is (C₆-C₁₀)aryl(C₁-C₆)alkyl-, including, without limitation, benzyl, phenethyl, and naphthylmethyl. An example of a substituted araC1-C6 alkyl is wherein the alkyl group is substituted with hydroxyalkyl.

A “heterocyclyl” or “heterocyclic” group is a ring structure having from 3 to 12 atoms, for example 4 to 8 atoms, wherein one or more atoms are selected from the group consisting of N, O, and S wherein the ring N atom may be oxidized to N—O, and the ring S atom may be oxidized to SO or SO₂, the remainder of the ring atoms being carbon. The heterocyclyl may be a monocyclic, a bicyclic, a spirocyclic or a bridged ring system. The heterocyclic group is optionally substituted with one or more R⁸ or R⁹ groups on ring carbon or ring nitrogen at one or more positions, wherein R⁶ is as defined for Formula I. The heterocyclic group is also independently optionally substituted on a ring nitrogen atom with alkyl, aralkyl, alkylcarbonyl, or on sulfur with lower alkyl. Examples of heterocyclic groups include, without limitation, epoxy, azetidinyl, aziridinyl, tetrahydrofuranyl, tetrahydropyranyl, pyrrolidinyl, pyrrolidinonyl, piperidinyl, piperazinyl, imidazolidinyl, imidazopyridinyl, thiazolidinyl, dithianyl, trithianyl, dioxolanyl, oxazolidinyl, oxazolidinonyl, decahydroquinolinyl, piperidonyl, 4-piperidinonyl, quinuclidinyl, thiomorpholinyl, thiomorpholinyl 1,1 dioxide, morpholinyl, azepanyl, oxazepanyl, azabicyclohexanyls, azabicycloheptanyl, azabicyclooctanyls, azabicyclononanyls (e.g., octahydroindolizinyl), azaspiroheptanyls, dihydro-1H,3H,5H-oxazolo[3,4-c]oxazolyl, tetrahydro-1′H,3′H-spiro[cyclopropane-1,2′-pyrrolizine], hexahydro-1H-pyrrolizinyl, hexahydro-1H-pyrrolo[2,1-c][1,4]oxazinyl, octahydroindolizinyl, oxaazaspirononanyls, oxaazaspirooctanyls, diazaspirononanyls, oxaazabiocycloheptanyls, hexahydropyrrolizinyl 4(1H)-oxide, tetrahydro-2H-thiopyranyl 1-oxide and tetrahydro-2H-thiopyranyl 1,1-dioxide. Specifically excluded from the scope of this term are compounds having adjacent annular O and/or S atoms.

As used herein, the term “heteroaryl” refers to groups having 5 to 14 ring atoms, preferably 5, 6, 9, or 10 ring atoms; having 6, 10, or 14π electrons shared in a cyclic array; and having, in addition to carbon atoms, from one to three heteroatoms per ring, or from one to three heteroatoms in at least one ring, selected from the group consisting of N, O, and S. Examples of heteroaryl groups include acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, 6,7-dihydro-5H-pyrrolo[1,2-a]imidazole, furanyl, furazanyl, imidazolinyl, imidazolyl, 1H-indazolyl, indolenyl, indolinyl, indolizinyl, indolyl, 3H-indolyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinyl, pyrimidinyl, phenanthridinyl, phenanthrolinyl, phenazinyl, phenothiazinyl, phenoxathiinyl, phenoxazinyl, phthalazinyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolinyl, 2H-pyrrolyl, pyrrolyl, quinazolinyl, quinolinyl, 4H-quinolizinyl, quinoxalinyl, quinuclidinyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. “Heteroaryl” also refers to bicyclic ring systems having, in addition to carbon atoms, from one to three heteroatoms per ring selected from the group consisting of N, O, and S in which one ring system may be saturated or partially saturated.

As used herein, “an effective amount” of a compound is an amount that is sufficient to negatively modulate or inhibit the activity of one or more of wild type Kras, Kras G12A, Kras G12C, Kras G12D, Kras G12R, Kras G12S, Kras G12V, Kras G13D or Kras Q61H. Such amount may be administered as a single dosage or may be administered according to a regimen, whereby it is effective.

As used herein, a “therapeutically effective amount” of a compound is an amount that is sufficient to ameliorate, or in some manner reduce a symptom or stop or reverse progression of a condition, or negatively modulate or inhibit the activity of one or more of wild type Kras, Kras G12A, Kras G12C, Kras G12D, Kras G12R, Kras G12S, Kras G12V, Kras G13D or Kras Q61H. Such amount may be administered as a single dosage or may be administered according to a regimen, whereby it is effective.

As used herein, treatment means any manner in which the symptoms or pathology of a condition, disorder or disease are ameliorated or otherwise beneficially altered. Treatment also encompasses any pharmaceutical use of the compositions herein.

As used herein, amelioration of the symptoms of a particular disorder by administration of a particular pharmaceutical composition refers to any lessening, whether permanent or temporary, lasting or transient that can be attributed to or associated with administration of the composition.

### Compounds

In one embodiment of the invention there are provided compounds of Formula (I):

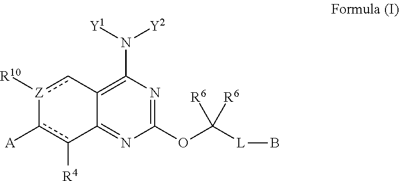

or a pharmaceutically acceptable salt thereof, wherein:

A is aryl or heteroaryl, wherein the aryl or the heteroaryl is optionally substituted with 1-4 R¹;

B is:

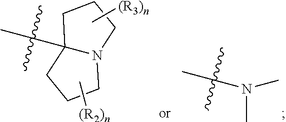

Y¹ is hydrogen, hydroxy, halogen, C1-C4 alkyl, L-SO₂—NH₂, L-OH, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle optionally substituted with 1-2 oxo (═O) or oxo-containing substituent, and optionally further substituted with 1-2 R⁸;

Y² is hydrogen or C1-C4 alkyl;

or Y¹ and Y² join to form:

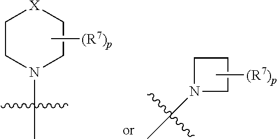

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—;

each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;

each R² is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —OC(O)N(R⁵)₂, —CO₂R⁵, or —CO₂N(R⁵)₂;

each R³ is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —COC(O)N(R⁵)₂, —CO₂R⁵, or —CO₂N(R⁵)₂;

R⁴ is hydrogen, halogen or C1-C3 alkyl;

each R⁵ is independently hydrogen or C1-C3 alkyl;

each R⁶ is independently hydrogen, hydroxy, C1-C4 hydroxyalkyl or heteroaryl;

each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, aryl, —CH₂—S(O)₂NH₂, or heteroaryl optionally independently substituted with 1-2 C1-C3 alkyl, —CN or C(O)NH₂,

two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),

two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and

two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;

each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;

each R⁹ is independently C1-C3 alkyl, hydroxy, halogen, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;

R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;

L is a bond, —C1-C4 alkyl-, —NH—, —N(C1-C3 alkyl)- or cyclopropyl-CH₂—;

Z is C or O, wherein if Z is C the 6-membered ring that includes Z is aromatic, and wherein if Z is O the 6-membered ring that includes Z is an oxane;

each n is 0-3;

o is 1-6; and

p is 1-8.

Such embodiment can include compounds or salts wherein:

A is aryl, optionally substituted with 1-4 R¹;

Y¹ and Y² join to form:

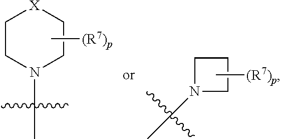

where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—;

each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, or (C1-C3 alkoxy)haloC1-C3 alkyl-;

each R⁶ is independently hydrogen, hydroxy, C1-C4 hydroxyalkyl or heteroaryl;

each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, or —CH₂—S(O)₂NH₂,

two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),

two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and

two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;

each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;

R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;

L is a bond, —C1-C4 alkyl-, —NH—, —N(C1-C3 alkyl)- or cyclopropyl-CH₂—;

Z is C or O, wherein if Z is C the 6-membered ring that includes Z is aromatic, and wherein if Z is O the 6-membered ring that includes Z is an oxane;

each n is 0-3; and

p is 1-8.

In some embodiments described herein, B is:

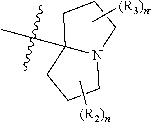

In some embodiments described herein B is di-methyl amino.

In some embodiments, A is naphthyl.

In certain embodiments, -L-B is:

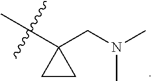 [SMILES: *C1(CN(C)C)CC1]

In certain embodiments, A is indazolyl.

In certain embodiments, A is benzothiophenyl.

In some embodiments described herein, at least one R¹ is C1-C4 alkyl.

In certain embodiments described herein, at least one R¹ is halogen, and is preferably fluorine.

In some embodiments described herein, at least one R¹ is hydroxy.

In some embodiments described herein, at least one R² is halogen, and is preferably fluorine.

In certain embodiments of the invention, at least one R³ is halogen, and is preferably fluorine.

In certain embodiments of the invention, at least one R³ is selected from the group consisting of ethenyl, fluoro ethenyl, and di-fluoro ethenyl.

In certain embodiments of the invention, R⁴ is halogen, and is preferably fluorine.

In certain embodiments of the invention, one or both R⁶ are C1-C4 alkyl.

In certain embodiments of the invention, one or both R⁶ are hydrogen.

In certain embodiments of the invention described herein, two R⁷ on the same atom join to form a spirocyclic ring selected from C3-C6 cycloalkyl and heterocycle, where said spirocyclic ring is optionally substituted with one or more substituents selected from oxo (═O), halogen, hydroxy, C1-C3 alkyl and —O—(C1-C3 alkyl).

In certain embodiments of the invention described herein, two R⁷ on adjacent atoms join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R⁸.

In certain embodiments of the invention, two R⁷ on non-adjacent atoms join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;

In certain embodiments of the invention, at least one R⁸ is C1-C4 alkyl.

In certain embodiments of the invention, at least one R⁸ is hydroxy or C1-C3 alkyl-hydroxy.

In certain embodiments of the invention, one or two R⁸ are oxo (═O).

In certain embodiments of the invention, Y¹ and Y² join to form piperidine, azepane, azocane, thiazepine, diazepane, oxazepane, azetidine, pyrrolidine, piperazine bound to a fused ring via nitrogen or thiomorpholine.

Non-limiting examples of compounds of Formula (I) are selected from the group consisting of:

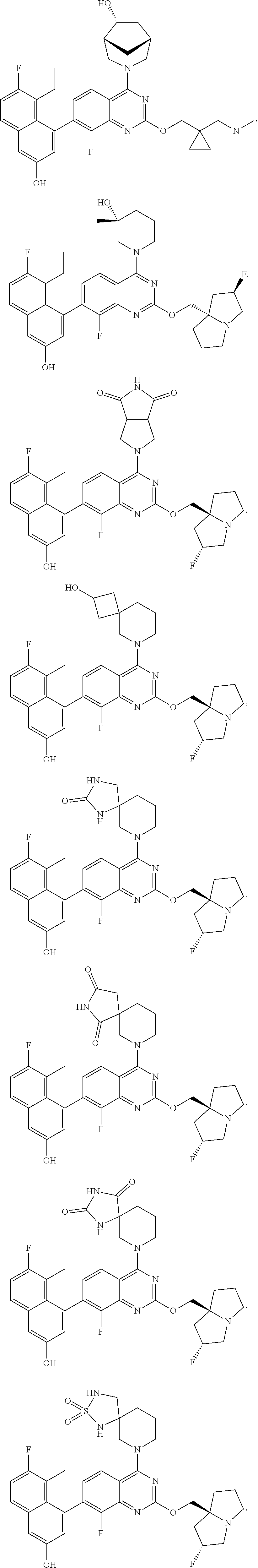

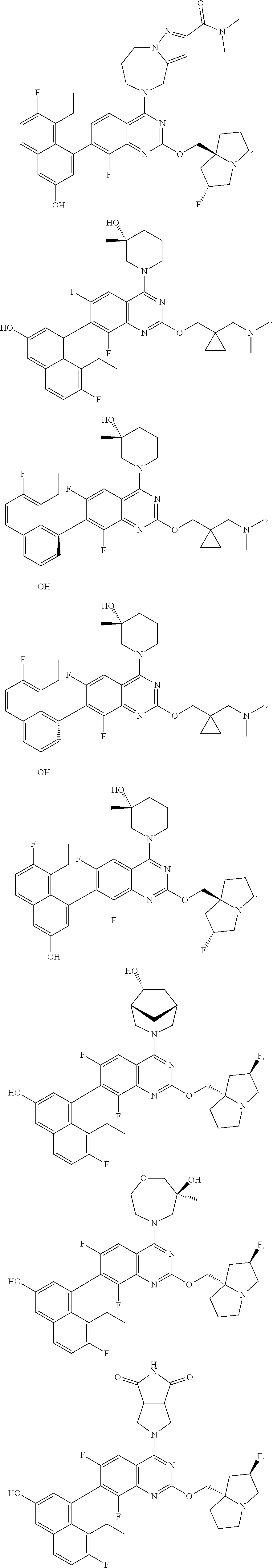

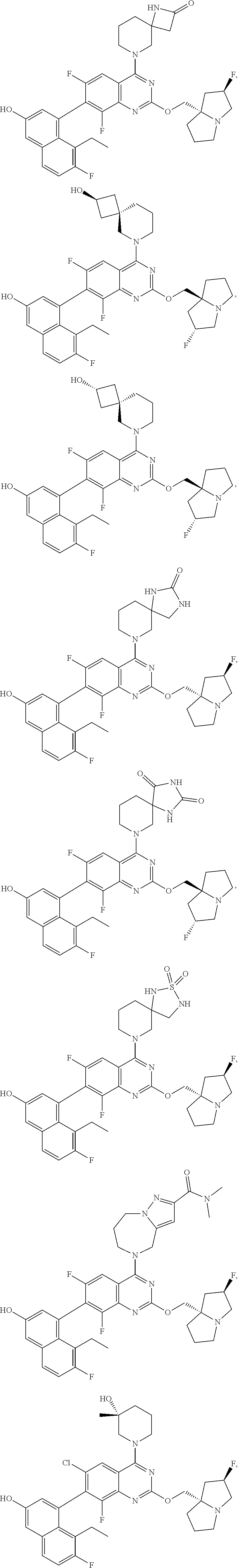

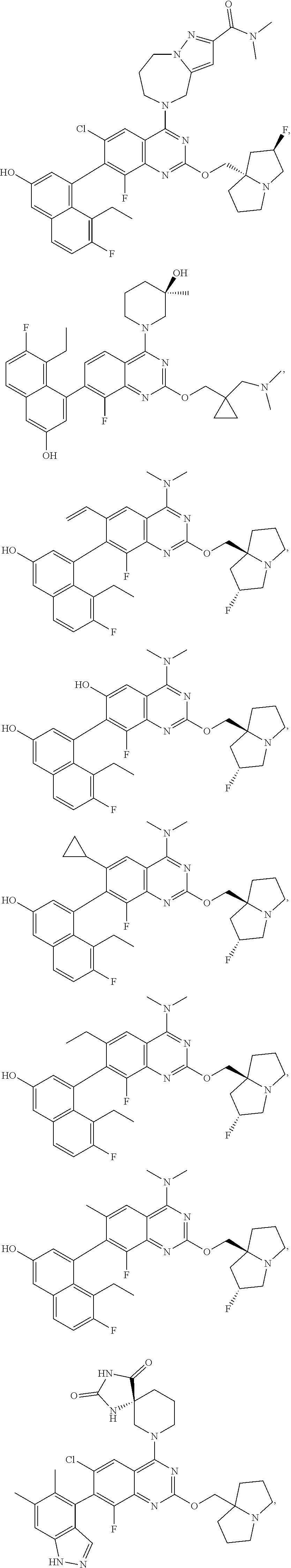

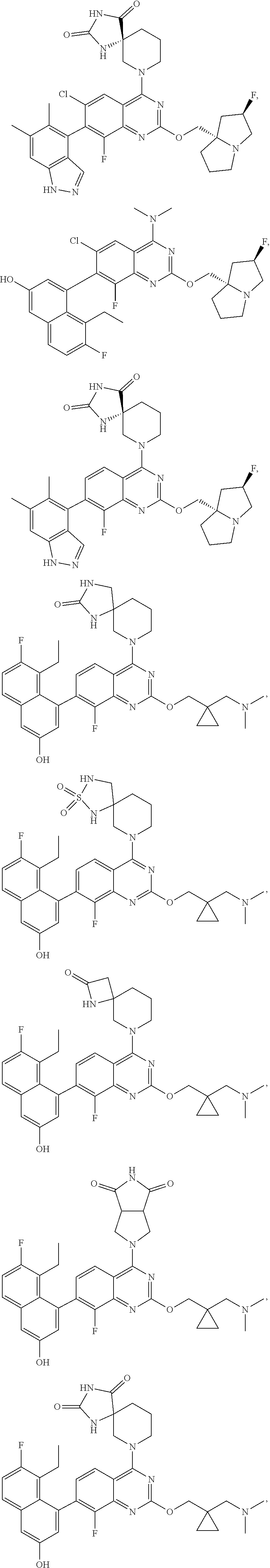

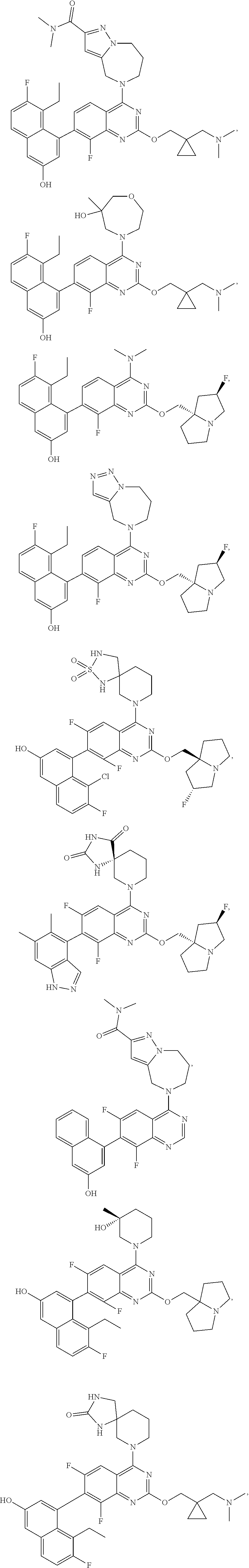

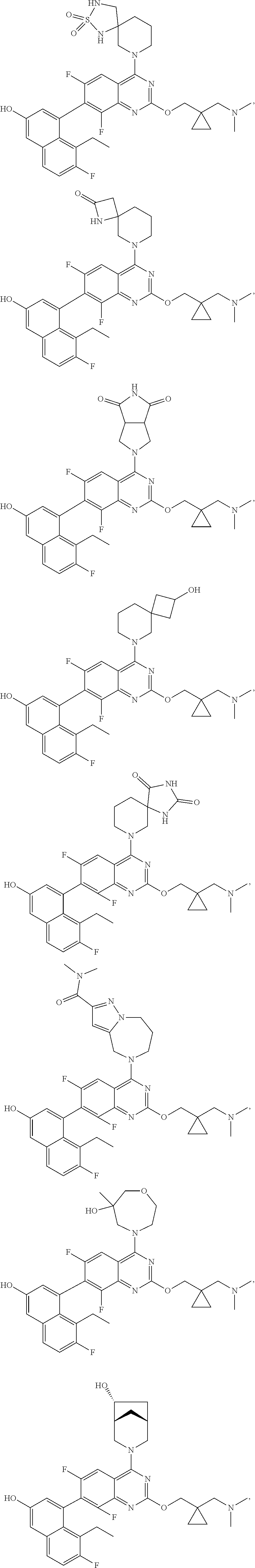

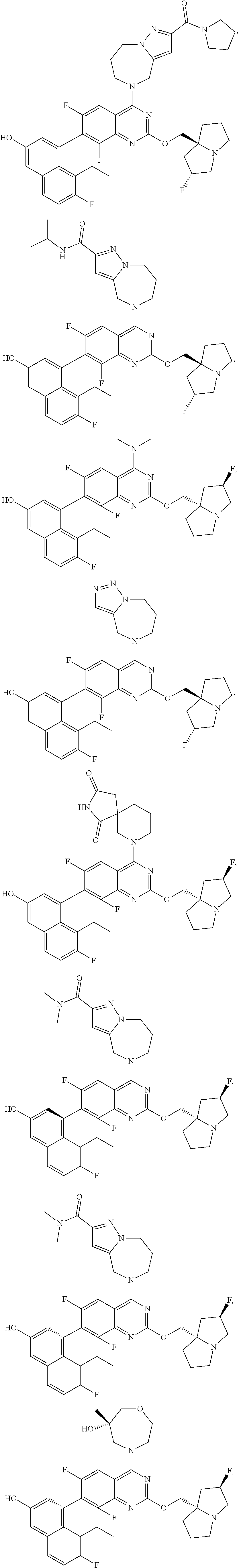

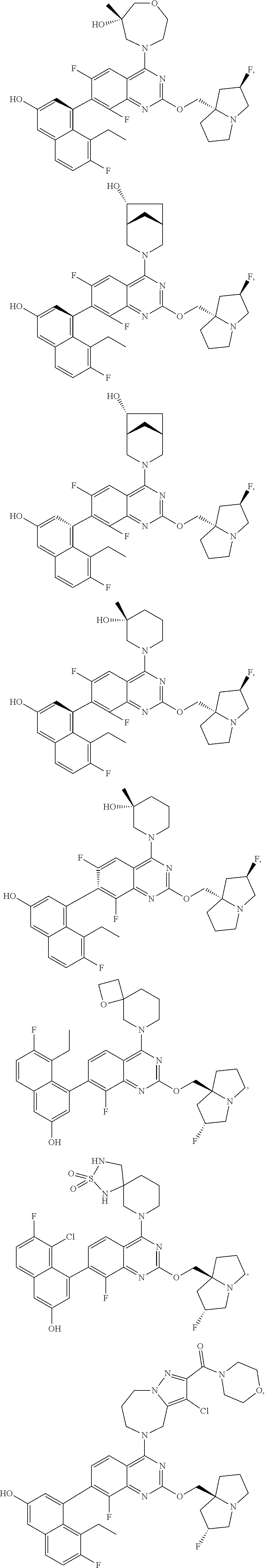

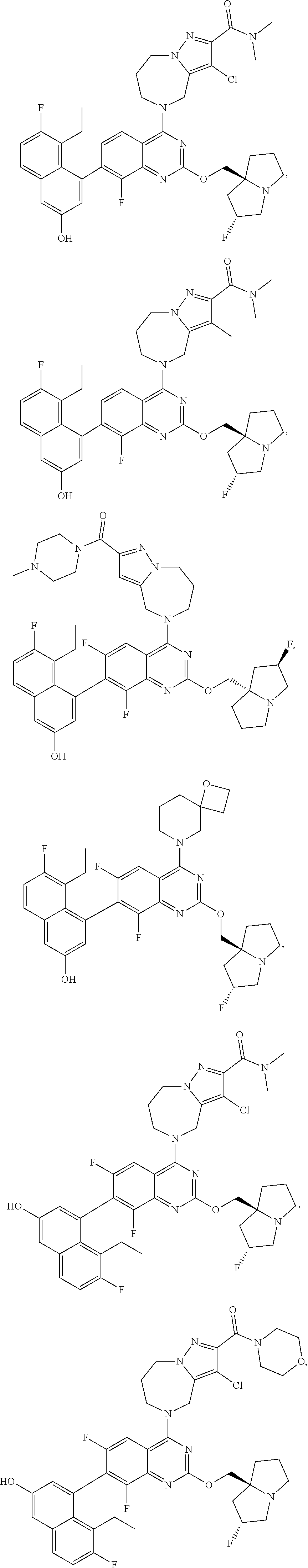

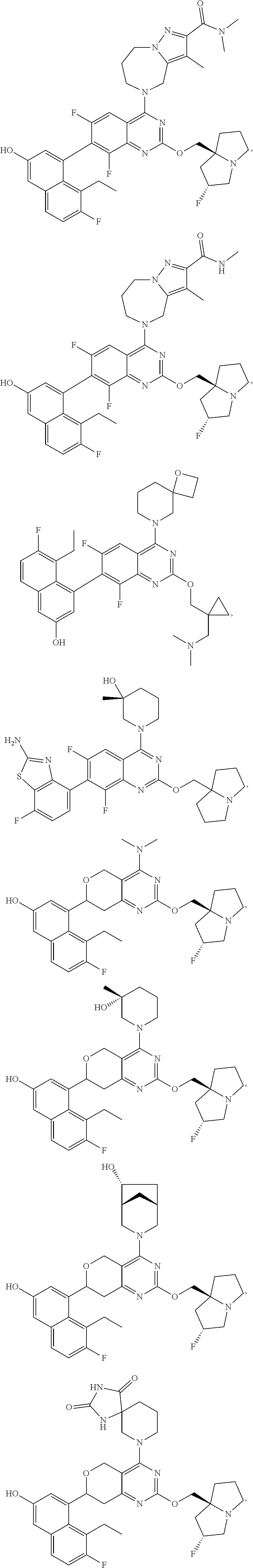

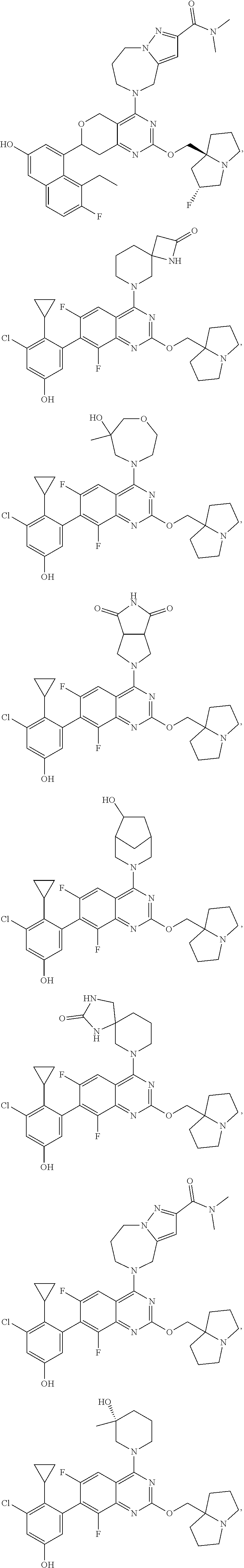

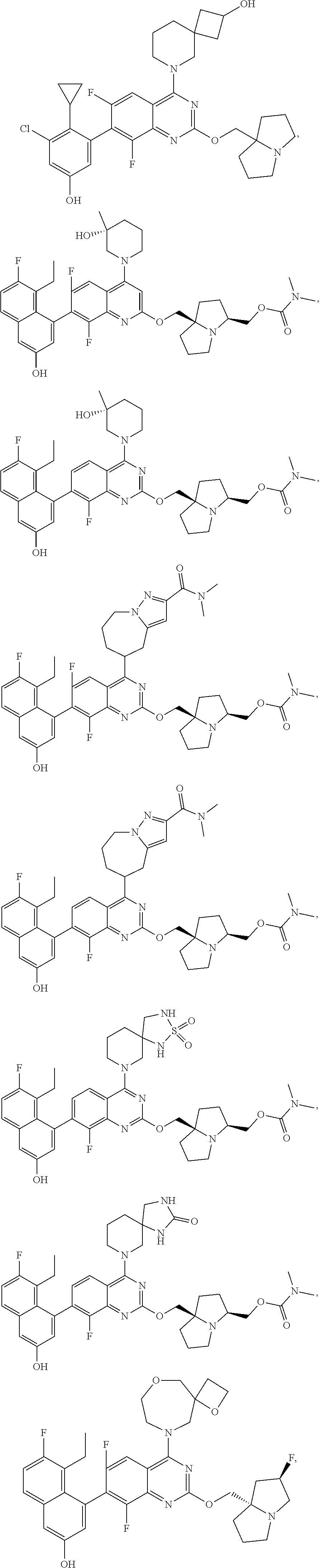

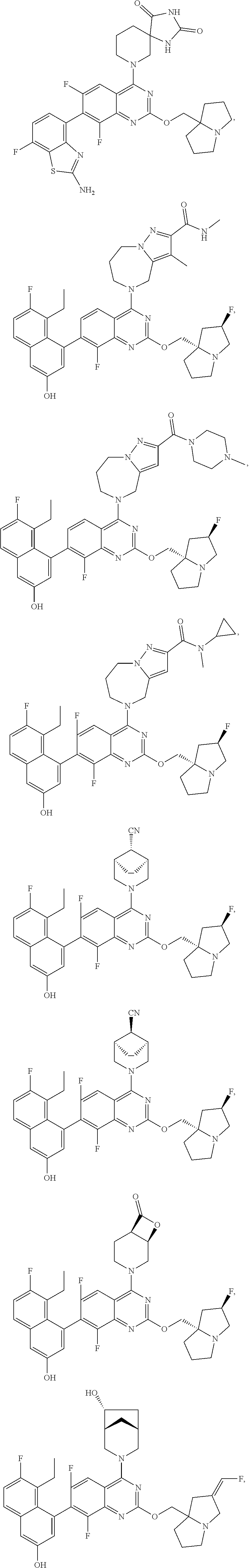

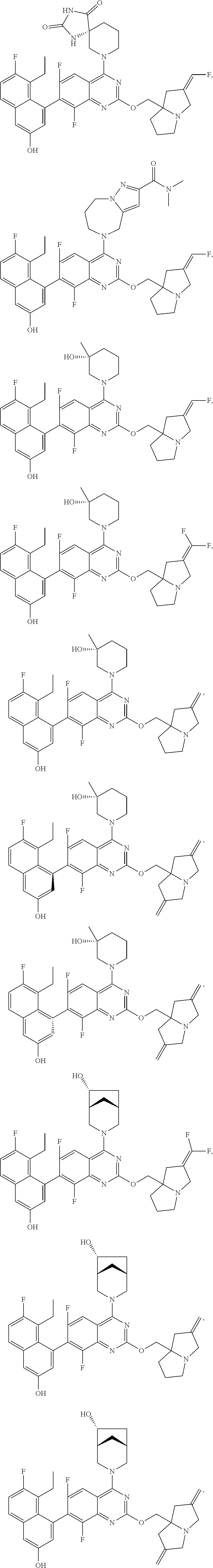

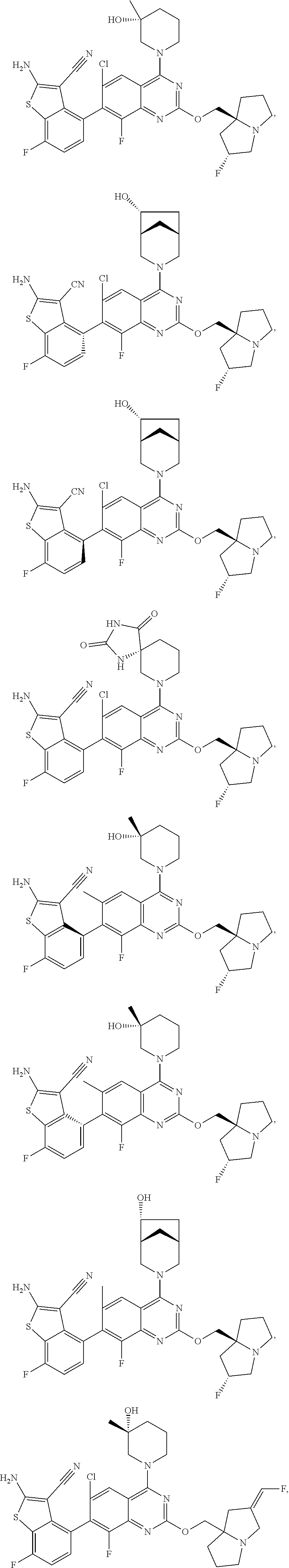

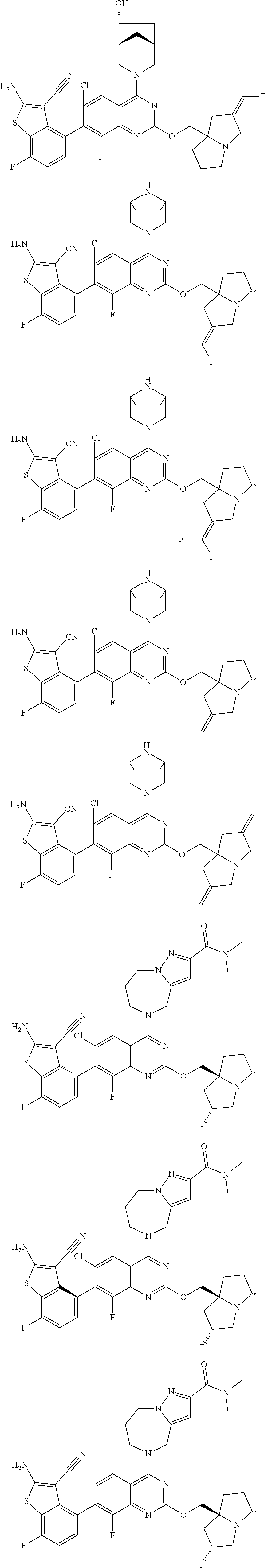

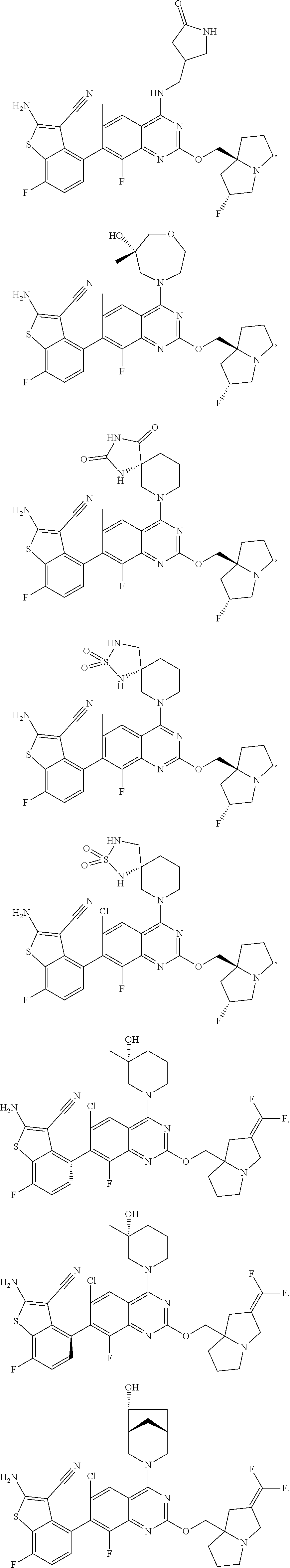

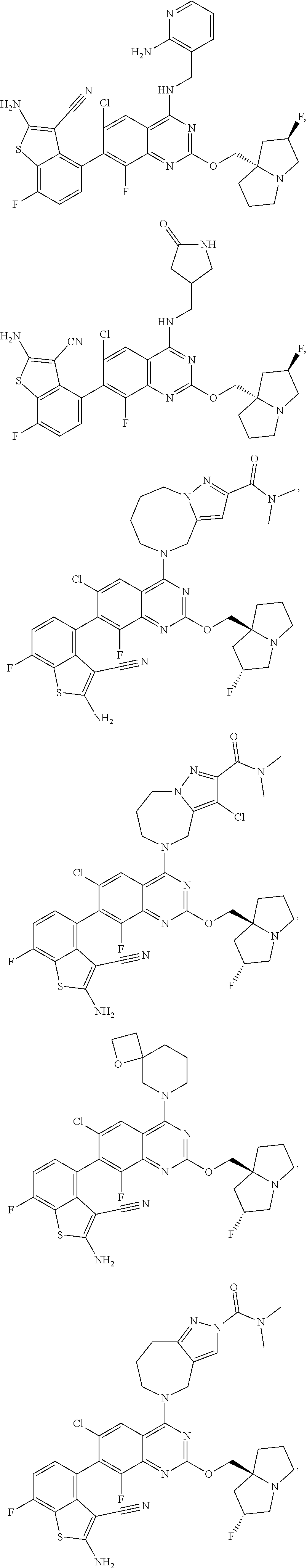

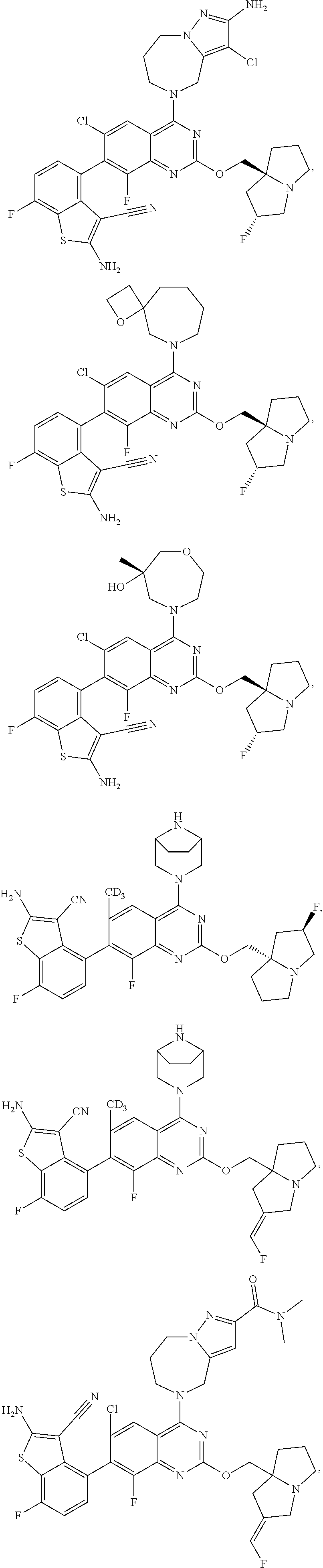

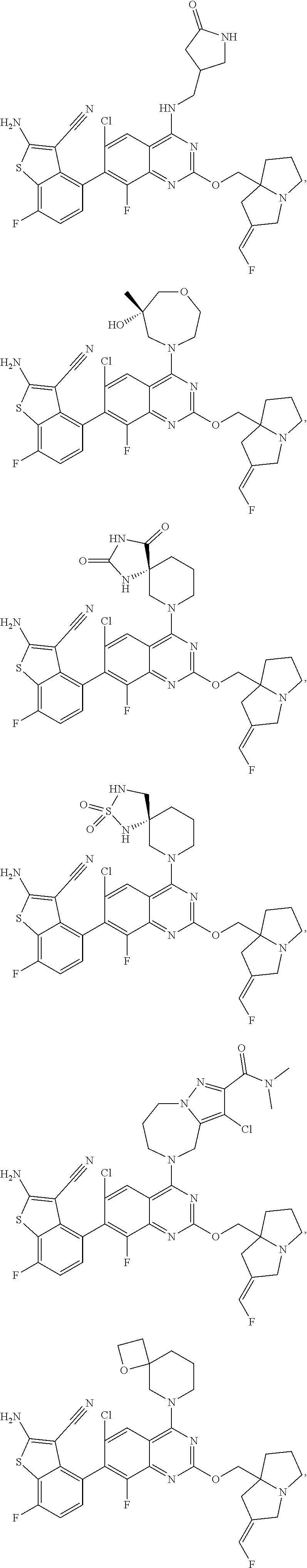

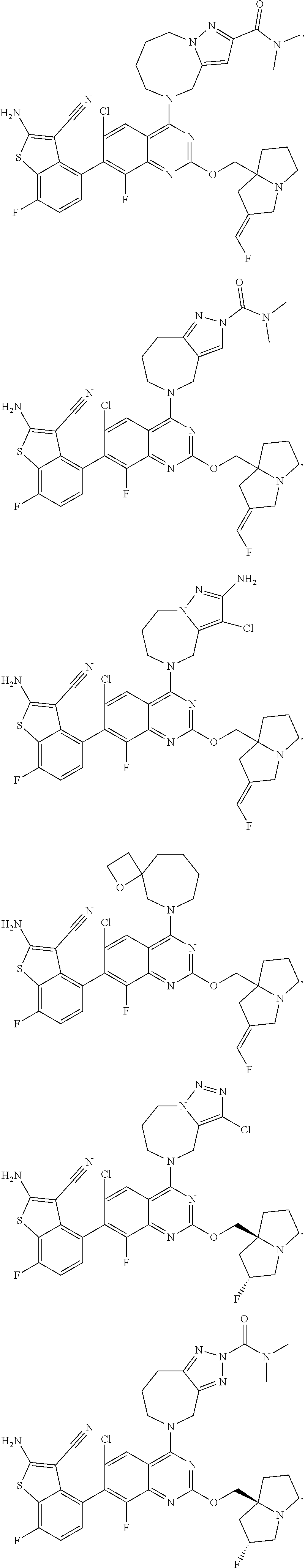

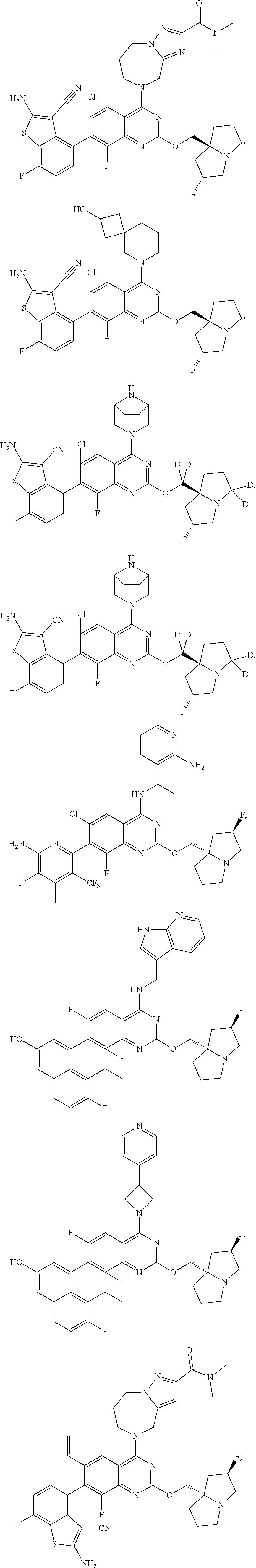

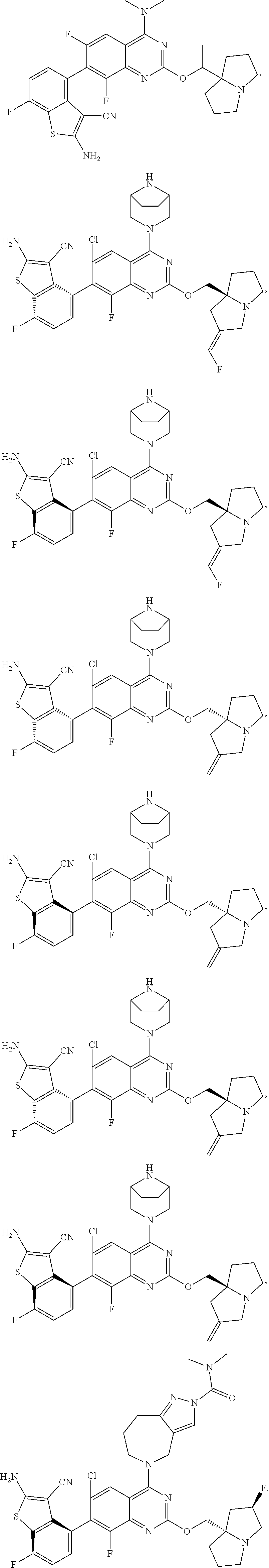

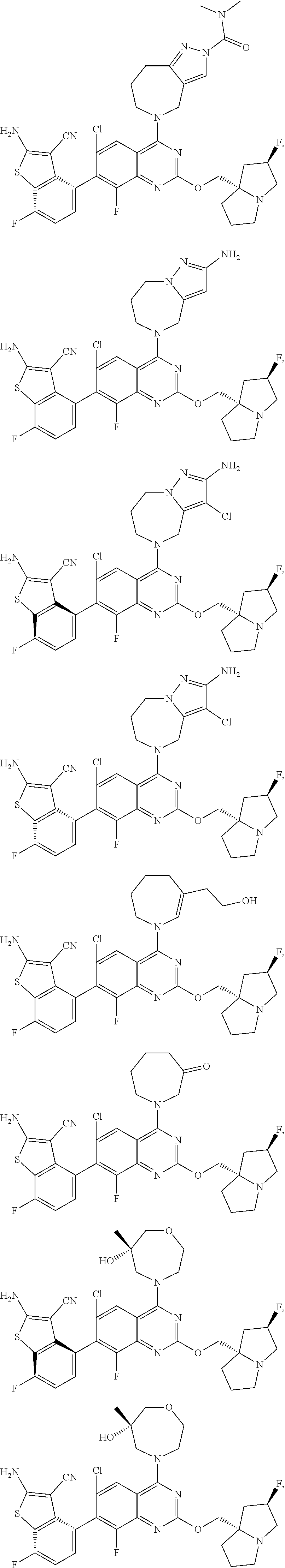

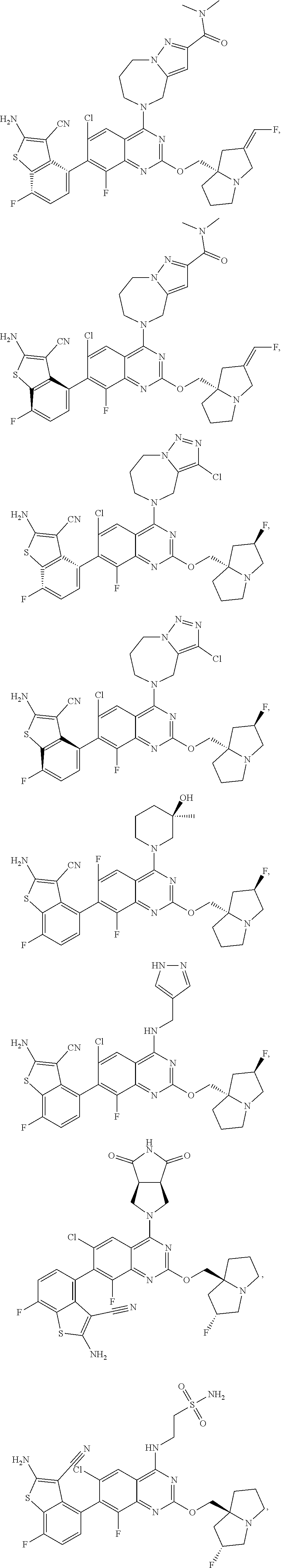

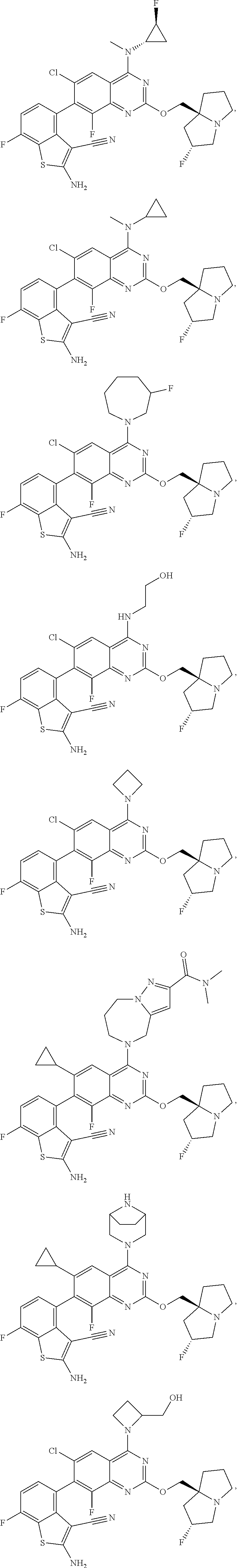

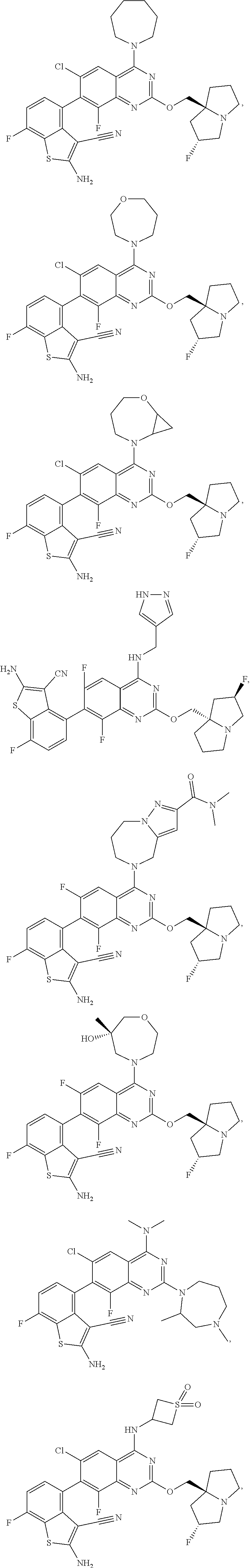

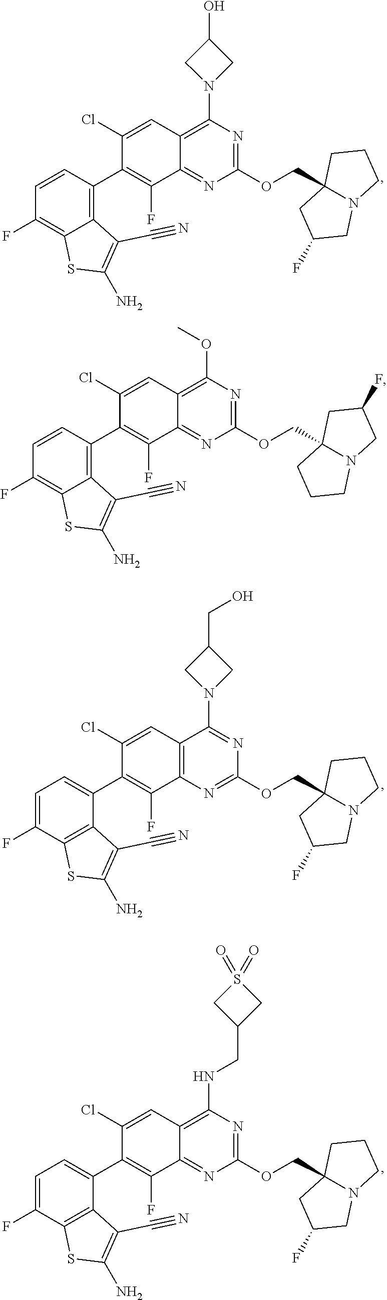 [SMILES: N#Cc1c(N)sc2c(F)ccc(-c3c(Cl)cc4c(N5CC(O)C5)nc(OC[C@@]56CCCN5C[C@H](F)C6)nc4c3F)c12 ; COc1nc(OC[C@@]23CCCN2C[C@H](F)C3)nc2c(F)c(-c3ccc(F)c4sc(N)c(C#N)c34)c(Cl)cc12 ; N#Cc1c(N)sc2c(F)ccc(-c3c(Cl)cc4c(N5CC(CO)C5)nc(OC[C@@]56CCCN5C[C@H](F)C6)nc4c3F)c12 ; N#Cc1c(N)sc2c(F)ccc(-c3c(Cl)cc4c(NCC5CS(=O)(=O)C5)nc(OC[C@@]56CCCN5C[C@H](F)C6)nc4c3F)c12]

and pharmaceutically acceptable salts thereof.

In one embodiment, the compounds of Formula (I) include bis-hydrochloride, tris-hydrochloride, trifluoroacetic acid, bis-trifluoroacetic acid, and tris-trifluoracetic acid salts of the above compounds. The compounds of Formula (I) or pharmaceutically acceptable salt thereof may be formulated into pharmaceutical compositions.

### Pharmaceutical Compositions

In another aspect, the invention provides pharmaceutical compositions comprising a wild type Kras, Kras G12A, Kras G12C, Kras G12D, Kras G12R, Kras G12S, Kras G12V, Kras G13D and/or Kras Q61H inhibitor according to the invention and a pharmaceutically acceptable carrier, excipient, or diluent. Compounds of the invention may be formulated by any method well known in the art and may be prepared for administration by any route, including, without limitation, parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intranasal, intratracheal, intrarectal, subcutaneous, and topical administration. In certain embodiments, compounds of the invention are administered intravenously in a hospital setting. In one embodiment, administration may be by the oral route. In some embodiments, the provided pharmaceutical compositions may be administered to a subject in need of treatment by injection systemically, such as by intravenous injection; or by injection or application to the relevant site, such as by direct injection via syringe, or direct application to the site when the site is exposed in surgery; or by topical administration.

Parenteral administration can be by bolus injection or continuous infusion. Pharmaceutical compositions for injection may be presented in unit dosage form, e.g., in ampoules or in multi-dose containers, with an added preservative.

The provided pharmaceutical compositions can also be formulated as a depot preparation. Such long acting formulations may be administered by implantation (for example subcutaneously or intramuscularly) or by intramuscular injection. Thus, for example, the formulations may be modified with suitable polymeric or hydrophobic materials (for example as an emulsion in an acceptable oil) or ion exchange resins, or as sparingly soluble derivatives, for example, as a sparingly soluble salt.

The pharmaceutical compositions may, if desired, be presented in a vial, pack or a medical device, including but not limited to a dispenser device which may contain one or more unit dosage forms containing the active ingredient. In one embodiment the dispenser device can comprise a syringe having a single dose of the liquid formulation ready for injection. The syringe can be accompanied by instructions for administration.

The characteristics of the carrier will depend on the route of administration. As used herein, the term “pharmaceutically acceptable” means a non-toxic material that is compatible with a biological system such as a cell, cell culture, tissue, or organism, and that does not interfere with the effectiveness of the biological activity of the active ingredient(s). Thus, compositions according to the invention may contain, in addition to the inhibitor, diluents, fillers, salts, buffers, stabilizers, solubilizers, and other materials well known in the art. The preparation of pharmaceutically acceptable formulations is described in, e.g., Remington's Pharmaceutical Sciences, 1^{8t}h Edition, ed. A. Gennaro, Mack Publishing Co., Easton, Pa., 1990.

As used herein, the term pharmaceutically acceptable salt refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counterion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The active compound is included in the pharmaceutically acceptable carrier or diluent in an amount sufficient to deliver to a patient a therapeutically effective amount without causing serious toxic effects in the patient treated. In one embodiment, a dose of the active compound for all of the above-mentioned conditions is in the range from about 0.01 to 300 mg/kg, for example 0.1 to 100 mg/kg per day, and as a further example 0.5 to about 25 mg per kilogram body weight of the recipient per day. A typical topical dosage will range from 0.01-3% wt/wt in a suitable carrier. The effective dosage range of the pharmaceutically acceptable derivatives can be calculated based on the weight of the parent compound to be delivered. If the derivative exhibits activity in itself, the effective dosage can be estimated as above using the weight of the derivative, or by other means known to those skilled in the art.

The pharmaceutical compositions comprising compounds of the present invention may be used in the methods of use described herein.

### Methods of Use

In yet another aspect, the invention provides for methods for inhibiting wild type kRas, kRas G12A, kRas G12C, kRas G12D, kRas G12R, kRas G12S, kRas G12V and/or kRas Q61H activity in a cell, comprising contacting the cell in which inhibition of wild type kRas, kRas G12A, kRas G12C, kRas G12D, kRas G12R, kRas G12S, kRas G12V and/or Q61H activity is desired with an effective amount of a compound of Formula (I), pharmaceutically acceptable salts thereof, or pharmaceutical compositions containing the compound or pharmaceutically acceptable salt thereof. In one embodiment, the contacting is in vitro. In one embodiment, the contacting is in vivo.

As used herein, the term “contacting” refers to the bringing together of indicated moieties in an in vitro system or an in vivo system. For example, “contacting” wild type kRas, kRas G12A, kRas G12C, kRas G12D, kRas G12R, kRas G12S, kRas G12V, kRas G13D and/or kRas Q61H with a compound provided herein includes the administration of a compound provided herein to an individual or patient, such as a human, having wild type kRas or a kRas G12A, kRas G12C, kRas G12D, kRas G12R, kRas G12S, kRas G12V, kRas G13D and/or kRas Q61H mutation, as well as, for example, introducing a compound provided herein into a sample containing a cellular or purified preparation containing wild type kRas or a kRas G12A, kRas G12C, kRas G12D, kRas G12R, kRas G12S, kRas G12V, kRas G13D or kRas Q61H mutation.

In one embodiment, a cell in which inhibition of wild type kRas or kRas G12A, kRas G12C, kRas G12D, kRas G12R, kRas G12S, kRas G12V, kRas G13D and/or kRas Q61H activity is desired is contacted with an effective amount of a compound of Formula (I) or pharmaceutically acceptable salt thereof to negatively modulate the activity of one or more of wild type kRas or kRas G12A, kRas G12C, kRas G12D, kRas G12R, kRas G12S, kRas G12V, kRas G13D and kRas Q61H.

By negatively modulating the activity of one or more of wild type kRas or kRas G12A, kRas G12C, kRas G12D, kRas G12R, kRas G12S, kRas G12V, kRas G13D and kRas Q61H, the methods described herein are designed to inhibit undesired cellular proliferation resulting from enhanced wild type kRas or kRas G12A, kRas G12C, kRas G12D, kRas G12R, kRas G12S, kRas G12V, kRas G13D and/or kRas Q61H activity within the cell. The cells may be contacted in a single dose or multiple doses in accordance with a particular treatment regimen to affect the desired negative modulation of wild type kRas or kRas G12A, kRas G12C, kRas G12D, kRas G12R, kRas G12S, kRas G12V, kRas G13D and/or kRas Q61H. The ability of compounds to bind one or more of wild type kRas or kRas G12A, kRas G12C, kRas G12D, kRas G12R, kRas G12S, kRas G12V, kRas G13D and kRas Q61H may be monitored in vitro using well known methods, including those described in Examples A and B below. In addition, the inhibitory activity of exemplary compounds in cells may be monitored, for example, by measuring the inhibition of one or more of wild type kRas or kRas G12A, kRas G12C, kRas G12D, kRas G12R, kRas G12S, kRas G12V, kRas G13D and/or kRas Q61H activity of the amount of phosphorylated ERK, for example using the method described in Example C below.

In another aspect, methods of treating cancer in a patient in need thereof, comprising administering to said patient a therapeutically effective amount of a compound of Formula (I) or a pharmaceutically acceptable salt thereof, or a pharmaceutical composition comprising the compound or pharmaceutically acceptable salt thereof are provided.

The compositions and methods provided herein may be used for the treatment of a wild type kRas-associated or kRas G12A, kRas G12C, kRas G12D, kRas G12R, kRas G12S, kRas G12V, kRas G13D and/or kRas Q61H-associated cancer in a patient in need thereof, comprising administering to said patient a therapeutically effective amount of a compound of Formula (I), a pharmaceutically acceptable salt thereof, or a pharmaceutical composition comprising the compound or pharmaceutically acceptable salt thereof are provided. In one embodiment, the wild type kRas-associated or kRas G12A, kRas G12C, kRas G12D, kRas G12R, kRas G12S, kRas G12V, kRas G13D and/or kRas Q61H-associated cancer is lung cancer.

The compositions and methods provided herein may be used for the treatment of a wide variety of cancers including tumors such as lung, prostate, breast, brain, skin, cervical carcinomas, testicular carcinomas, etc. More particularly, cancers that may be treated by the compositions and methods of the invention include, but are not limited to tumor types such as astrocytic, breast, cervical, colorectal, endometrial, esophageal, gastric, head and neck, hepatocellular, laryngeal, lung, oral, ovarian, prostate and thyroid carcinomas and sarcomas. More specifically, these compounds can be used to treat: Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma. In certain embodiments, the cancer is non-small cell lung cancer, small cell lung cancer, colorectal cancer, rectal cancer or pancreatic cancer. In certain embodiments, the cancer is non-small cell lung cancer.

The concentration and route of administration to the patient will vary depending on the cancer to be treated. The compounds, pharmaceutically acceptable salts thereof and pharmaceutical compositions comprising such compounds and salts also may be co-administered with other anti-neoplastic compounds, e.g., chemotherapy, or used in combination with other treatments, such as radiation or surgical intervention, either as an adjuvant prior to surgery or post-operatively.

Also provided herein is a compound of Formula (I) or a pharmaceutically acceptable salt thereof, or a pharmaceutical composition thereof as defined herein for use in therapy.

Also provided herein is a compound of Formula (I) or a pharmaceutically acceptable salt thereof, or a pharmaceutical composition thereof as defined herein for use in the treatment of cancer.

Also provided herein is a compound of Formula (I) or a pharmaceutically acceptable salt thereof, or a pharmaceutical composition thereof for use in the inhibition of wild type kRas or kRas G12A, kRas G12C, kRas G12D, kRas G12R, kRas G12S, kRas G12V, kRas G13D and/or kRas Q61H.

Also provided herein is a compound of Formula (I) or a pharmaceutically acceptable salt thereof, or a pharmaceutical composition thereof as defined herein, for use in the treatment of wild type kRas-associated or a kRas G12A, kRas G12C, kRas G12D, kRas G12R, kRas G12S, kRas G12V, kRas G13D and/or kRas Q61H-associated disease or disorder.

Also provided herein is the use of a compound of Formula (I) or a pharmaceutically acceptable salt thereof, as defined herein in the manufacture of a medicament for the treatment of cancer.

Also provided herein is a use of a compound of Formula (I) or a pharmaceutically acceptable salt thereof, as defined herein in the manufacture of a medicament for the inhibition of activity of wild type kRas or kRas G12A, kRas G12C, kRas G12D, kRas G12R, kRas G12S, kRas G12V, kRas G13D and/or kRas Q61H.

Also provided herein is the use of a compound of Formula (I) or a pharmaceutically acceptable salt thereof, as defined herein, in the manufacture of a medicament for the treatment of wild type kRas-associated or a kRas G12A, kRas G12C, kRas G12D, kRas G12R, kRas G12S, kRas G12V, kRas G13D and/or kRas Q61H-associated disease or disorder.

Also provided herein is a method for treating cancer in a patient in need thereof, the method comprising (a) determining that cancer is associated with wild type kRas or a kRas G12A, kRas G12C, kRas G12D, kRas G12R, kRas G12S, kRas G12V, kRas G13D and/or kRas Q61H mutation (e.g., as determined using a regulatory agency-approved, e.g., FDA-approved, assay or kit); and (b) administering to the patient a therapeutically effective amount of a compound of Formula (I), or a pharmaceutically acceptable salt thereof, or a pharmaceutical composition thereof.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### REACTION SCHEMES AND EXAMPLES

The compounds of the present invention may be prepared from commercially available reagents using the synthetic methods and reaction schemes described herein, or using other reagents and conventional methods well known to those skilled in the art. For instance, compounds of the present invention may be prepared according to the reaction schemes and examples outlines below.

The compounds of the present invention may have one or more chiral center and may be synthesized as stereoisomeric mixtures, isomers of identical constitution that differ in the arrangement of their atoms in space. The compounds may be used as mixtures or the individual components/isomers may be separated using commercially available reagents and conventional methods for isolation of stereoisomers and enantiomers well-known to those skilled in the art, e.g., using CHIRALPAK® (Sigma-Aldrich) or CHIRALCEL® (Diacel Corp) chiral chromatographic HPLC columns according to the manufacturer's instructions. Alternatively, compounds of the present invention may be synthesized using optically pure, chiral reagents and intermediates to prepare individual isomers or enantiomers. Unless otherwise indicated, all chiral (enantiomeric and diastereomeric) and racemic forms are within the scope of the invention. Unless otherwise indicated, whenever the specification, including the claims, refers to compounds of the invention, the term “compound” is to be understood to encompass all chiral (enantiomeric and diastereomeric) and racemic forms.

The compounds of the present invention may be in anhydrous, solvated or hydrated forms, and all such forms are included within the scope of the invention.

The following Examples are intended to illustrate further certain embodiments of the invention and are not intended to limit the scope of the invention.

### Example 1

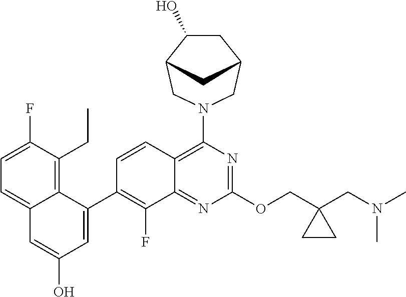 [SMILES: CCc1c(F)ccc2cc(O)cc(-c3ccc4c(N5C[C@@H]6C[C@H](C5)[C@H](O)C6)nc(OCC5(CN(C)C)CC5)nc4c3F)c12]

### (1R,5R,6R)-3-(2-((1-((dimethylamino)methyl)cyclopropyl)methoxy)-7-(8-ethyl-7-fluoro-3-hydroxynaphthalen-1-yl)-8-fluoroquinazolin-4-yl)-3-azabicyclo[3.2.1]octan-6-ol

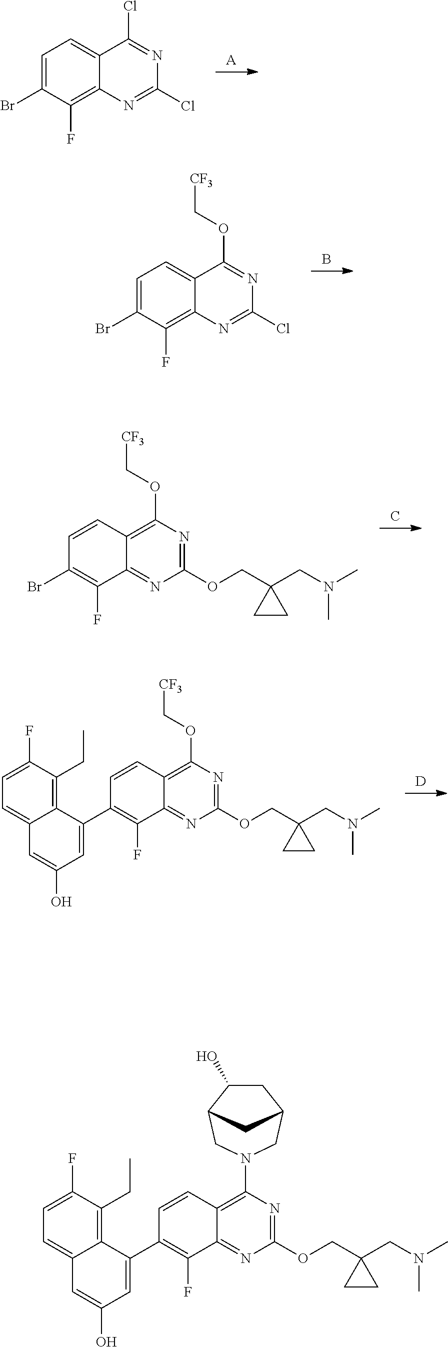

Step A. 7-bromo-2-chloro-8-fluoro-4-(2,2,2-trifluoroethoxy)quinazoline: To a solution of 2,2,2-trifluoroethanol (3.45 g, 1.2 equiv) in THE (36 mL) was added NaH (1.38 g, 60% purity, 1.2 equiv) at 0° C. The mixture was stirred at 0° C. for 0.5 hour. Then the mixture was added to a solution of 7-bromo-2,4-dichloro-8-fluoro-quinazoline (8.50 g, 1.0 equiv) in THE (64 mL) at −40° C. The mixture was stirred at −40° C. for 1 hour. The mixture was diluted with saturated NH₄C1 aqueous solution (80 mL) and extracted with EtOAc (100 mL×3). The organic layer was dried with anhydrous sodium sulfate, filtered and concentrated under vacuum. The residue was purified by column chromatography (SiO₂, petroleum ether/ethyl acetate=I/O to 5/1) and concentrated under vacuum to afford the title compound (9.10 g, 88% yield) as yellow solid; ¹H NMR (400 MHz, CDCl₃-d) δ=7.87 (dd, J=1.2, 8.8 Hz, 1H), 7.77 (dd, J=6.0, 8.8 Hz, 1H), 5.02 (q, J=8.0 Hz, 2H)

Step B. 1-[1-[[7-bromo-8-fluoro-4-(2,2,2-trifluoroethoxy)quinazolin-2-yl]oxymethyl]cyclopropyl]-N,N-dimethyl-methanamine: To a solution of 7-bromo-2-chloro-8-fluoro-4-(2,2,2-trifluoroethoxy)quinazoline (1.00 g, 1.0 equiv) and [1-[(dimethylamino)methyl]cyclopropyl]methanol (395 mg, 1.1 equiv) in dioxane (10 mL) was added Na₂CO₃ (884 mg, 3.0 equiv). The mixture was stirred at 40° C. for 12 hours. The mixture was diluted with water (3 mL) and extracted with EtOAc (5 mL×2). The combined organic layers were dried with anhydrous sodium sulfate and concentrated under vacuum. The residue was purified by column chromatography (SiO₂, PE/EtOAc=I/O to 0/1) and concentrated to afford the title compound (650 mg, 52% yield) as yellow oil; LCMS (ESI, M+3): m/z=454.1.

Step C. 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol: To a solution of 1-[1-[[7-bromo-8-fluoro-4-(2,2,2-trifluoroethoxy)quinazolin-2-yl]oxymethyl]cyclopropyl]-N,N-dimethylmethanamine (750 mg, 1.0 equiv) and 5-ethyl-6-fluoro-4-(4,4,5,5-tetramethyl-1,3,2-dioxaborolan-2-yl)naphthalen-2-ol (629 mg, 1.2 equiv) in CPME (7 mL) were added Cs₂CO₃ (1.5 M, 3.3 mL, 3.0 equiv) and Ad₂nBup-Pd-G₃ (242 mg, 0.2 equiv). The mixture was stirred at 100° C. for 2 hours under N₂ atmosphere. The mixture was diluted with water (5 mL) and extracted with EtOAc (8 mL×3). The organic layer was dried with anhydrous sodium sulfate and concentrated under vacuum. The residue was purified by column chromatography (SiO₂, PE/EtOAc=I/O to 0/1) and concentrated to afford the title compound (740 mg, 79% yield) as brown solid; LCMS (ESI, M+1): m/z=562.0.

Step D. (1S,5S,6S)-3-[2-[[1-[(dimethylamino)methyl]cyclopropyl]methoxy]-7-(8-ethyl-7-fluoro-3-hydroxy-1-naphthyl)-8-fluoro-quinazolin-4-yl]-3-azabicyclo[3.2.1]octan-6-ol: To a solution of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (250 mg, 1.0 equiv) and (1S,5S,6S)-3-azabicyclo[3.2.1]octan-6-ol (68.0 mg, 1.2 equiv) in DMF (1 mL) and AcN (1 mL) was added K₃PO₄ (283 mg, 3.0 equiv). The mixture was stirred at 40° C. for 12 hours. The mixture was diluted with water (3 mL) and extracted with EtOAc (3 mL×2). The organic layer was dried over anhydrous sodium sulfate and concentrated under vacuum. The residue was purified by prep-HPLC [Phenomenex Synergi C18 150×25 mm×10 um; A: water (FA), B: ACN, B %: 23%-53% over 10 min] and concentrated to remove ACN. The aqueous phase was adjusted to pH=7 with saturated NaHCO₃ aqueous solution and extracted with DCM (20 mL×3). The organic layer was dried over anhydrous sodium sulfate and concentrated. The impurity was further purified prep-HPLC [Phenomenex luna C18 150×25 mm×10 um; A: water (FA), B: ACN, B %: 17%-47% over 12 min] and lyophilized to afford the title compound (53.06 mg, 19% yield) as white solid; SFC: Rt=0.660 min, 1.511 min; column: Chiralpak IC-3 50×4.6 mm I.D., 3 um; mobile phase: phase A for CO₂, and phase B for EtOH (0.05% DEA); gradient elution: 50% EtOH (0.05% DEA) in CO₂; flow rate: 3 mL/min; detector: PDA; column temp: 35° C.; back pressure: 100 Bar. ¹H NMR (400 MHz, DMSO-d₆) δ=8.22-8.08 (m, 2H), 7.75 (dd, J=6.0, 8.8 Hz, 1H), 7.34 (t, J=9.4 Hz, 1H), 7.29 (d, J=1.6 Hz, 1H), 7.22 (br t, J=7.6 Hz, 1H), 6.93 (d, J=2.4 Hz, 1H), 4.63-4.48 (m, 2H), 4.26-4.13 (m, 3H), 3.52 (br t, J=10.2 Hz, 1H), 3.28 (br dd, J=7.2, 12.4 Hz, 1H), 2.40-2.25 (m, 6H), 2.21 (s, 6H), 2.13-2.03 (m, 2H), 1.76-1.60 (m, 2H), 1.36 (br d, J=13.2 Hz, 1H), 0.74-0.67 (m, 3H), 0.66-0.61 (m, 2H), 0.41 (s, 2H); LCMS (ESI, M+1): m/z=589.3.

### Example 2

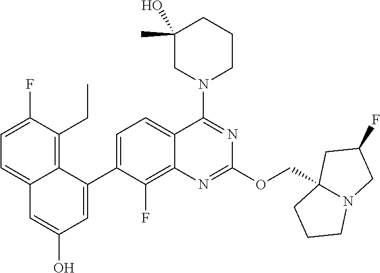 [SMILES: CCc1c(F)ccc2cc(O)cc(-c3ccc4c(N5CCC[C@@](C)(O)C5)nc(OC[C@@]56CCCN5C[C@H](F)C6)nc4c3F)c12]

### (R)-1-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-3-methylpiperidin-3-ol

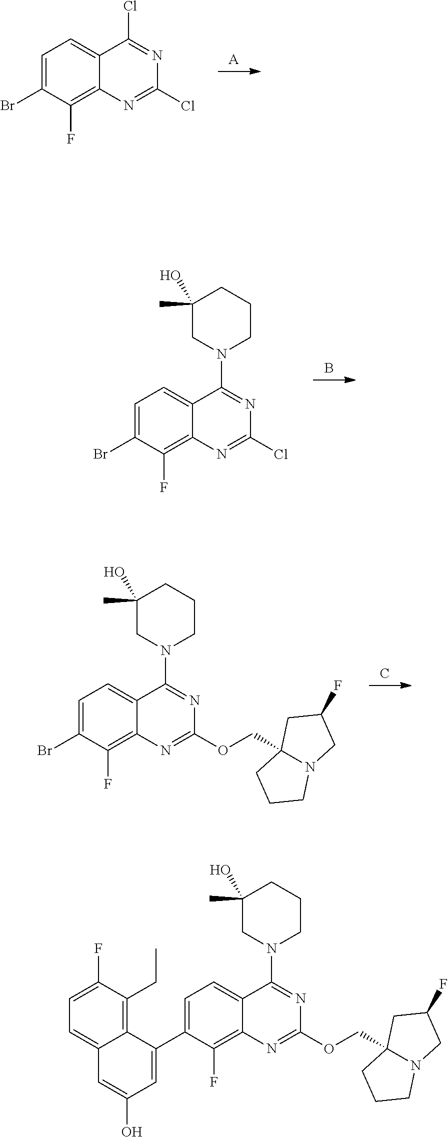

Step A. (R)-1-(7-bromo-2-chloro-8-fluoroquinazolin-4-yl)-3-methylpiperidin-3-ol: To a solution of 7-bromo-2,4-dichloro-8-fluoro-quinazoline (1.60 g, 1.0 equiv), DIEA (2.23 g, 3.19 equiv) and 4 Å molecular sieve (100 mg) in DCM (20 mL), (3R)-3-methylpiperidin-3-ol (1.0 g, 1.61 equiv) in DCM (5 mL). The mixture was stirred at 0˜15° C. for 16 hours. The mixture was diluted with water (200 mL), extracted with ethyl acetate (4×100 mL). The combined organic layers were dried over anhydrous sodium sulfate, filtered and concentrated under reduced pressure to give a residue. The residue was purified by column chromatography (SiO₂, Petroleum ether/Ethyl acetate=2/1 to 1/1) and TLC (PE/EA=1/1, Rf=0.49) to afford the tittle compound (1.0 g, 46% yield) as a red solid; LCMS (ESI, M+1): m/z=374.0.

Step B. (R)-1-(7-bromo-8-fluoro-2-(((2R,7aS)-2-fluorohexahydro-1H-pyrrolizin-7a-yl)methoxy)quinazolin-4-yl)-3-methylpiperidin-3-ol: To a solution of (3R)-1-(7-bromo-2-chloro-8-fluoro-quinazolin-4-yl)-3-methyl-piperidin-3-ol (400 mg, 1.0 equiv), [(2R,8S)-2-fluoro-1,2,3,5,6,7-hexahydropyrrolizin-8-yl]methanol (512 mg, 3.01 equiv) in dioxane (2.0 mL) were added DIEA (415 mg, 3.01 equiv) and 4 Å molecular sieve (80 mg). The mixture was stirred at 95° C. for 42 hours. The mixture was filtered. The filtrate was purified by reversed phase flash [water (FA, 0.1%)] to afford the tittle compound (260 mg, 43% yield) as a red solid; LCMS (ESI, M+1): m/z=499.2.

Step C. (R)-1-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-3-methylpiperidin-3-ol: A mixture of (R)-1-(7-bromo-8-fluoro-2-(((2R,7aS)-2-fluorotetrahydro-1H-pyrrolizin-7a(5H)-yl)methoxy)quinazolin-4-yl)-3-methylpiperidin-3-ol (100 mg, 1.0 equiv), 5-ethyl-6-fluoro-4-(4,4,5,5-tetramethyl-1,3,2-dioxaborolan-2-yl)naphthalen-2-ol (96.0 mg, 1.51 equiv) in Methoxycyclopentane (1.0 mL) was degassed and purged with N₂ for 3 times, [2-(2-aminophenyl)phenyl]palladium(1+);bis(1-adamantyl)-butyl-phosphane;methanesulfonate (14.6 mg, 0.1 equiv) was added and then the mixture was stirred at 95° C. for 3 hours under N₂ atmosphere. After completion, the mixture was diluted with water (3 mL) and extracted with ethyl acetate (4×4 mL). The combined organic layers were dried over anhydrous sodium sulfate, filtered and concentrated. The residue was purified by reversed phase flash [C18, 0.1% formic acid condition] and prep-HPLC [column: Phenomenex luna C18 150×25 mm×10 μm; mobile phase: water(0.225% FA)−ACN; B %: 15%-45% over 10 min] to afford the tittle compound (44.1 mg, 35% yield) as a white solid; ¹H NMR (400 MHz, METHANOL-d4) δ=7.99 (dd, J=1.6, 8.4 Hz, 1H), 7.65 (dd, J=6.0, 9.2 Hz, 1H), 7.33 (ddd, J=2.0, 6.8, 8.8 Hz, 1H), 7.27-7.19 (m, 2H), 6.95 (d, J=2.4 Hz, 1H), 5.54-5.37 (m, 1H), 4.58-4.52 (m, 1H), 4.51-4.45 (m, 1H), 4.32 (br d, J=12.8 Hz, 1H), 4.15 (br d, J=13.2 Hz, 1H), 3.84-3.68 (m, 1H), 3.53-3.52 (m, 1H), 3.84-3.52 (m, 2H), 3.51-3.42 (m, 1H), 3.28 (br d, J=4.4 Hz, 1H), 2.65-2.38 (m, 1H), 2.50-2.38 (m, 3H), 2.36-2.27 (m, 1H), 2.26-2.12 (m, 3H), 2.10-1.99 (m, 1H), 1.90-1.72 (m, 3H), 1.27 (d, J=13.1 Hz, 3H), 0.82-0.73 (m, 3H). LCMS (ESI, M+1): m/z=607.4.

### Example 3

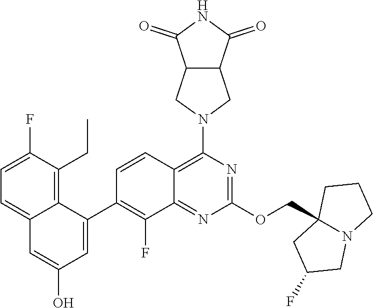 [SMILES: CCc1c(F)ccc2cc(O)cc(-c3ccc4c(N5CC6C(=O)NC(=O)C6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)tetrahydropyrrolo[3,4-c]pyrrole-1,3(2H,3aH)-dione

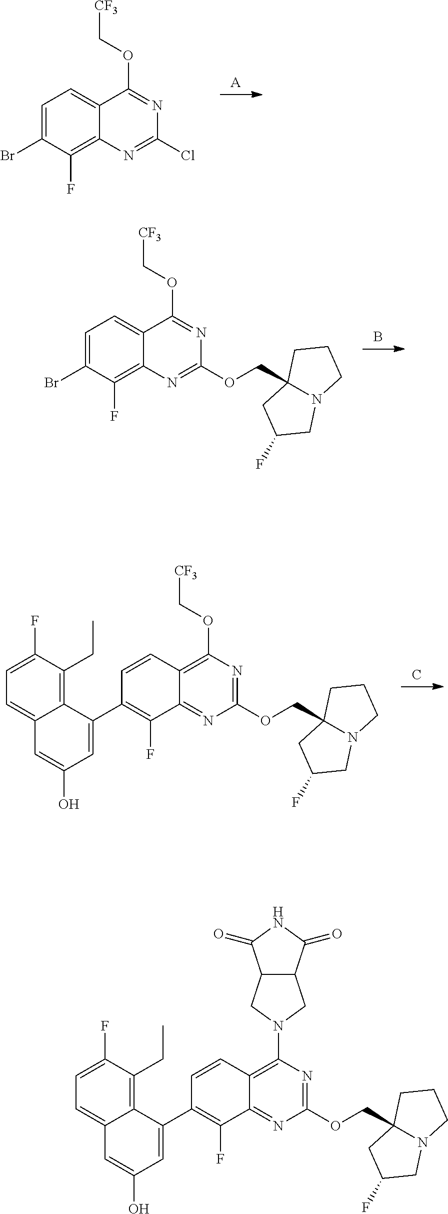

Step A. 7-bromo-8-fluoro-2-(((2R,7aS)-2-fluorohexahydro-1H-pyrrolizin-7a(5H)-yl)methoxy)-4-(2,2,2-trifluoroethoxy)quinazoline: To a solution of 7-bromo-2-chloro-8-fluoro-4-(2,2,2-trifluoroethoxy)quinazoline (5.00 g, 1.0 equiv) and [(2R,8S)-2-fluoro-1,2,3,5,6,7-hexahydropyrrolizin-8-yl]methanol (2.44 g, 1.1 equiv) in dioxane (50 mL) was added Na₂CO₃ (4.42 g, 3.0 equiv). The mixture was stirred at 110° C. for 12 hours. The mixture was filtered and washed with EtOAc (10 mL×2). The organic layer was concentrated under vacuum. The residue was purified by column chromatography (SiO₂, petroleum ether/ethyl acetate=I/O to 1/1) and concentrated under vacuum to afford the title compound (5.30 g, 79% yield) as yellow solid; ¹H NMR (400 MHz, CDCl₃-d) δ=7.75 (dd, J=1.6, 8.8 Hz, 1H), 7.51 (dd, J=6.0, 8.8 Hz, 1H), 5.36 (br d, J=1.6 Hz, 1H), 5.23 (br d, J=1.6 Hz, 1H), 4.97 (q, J=8.4 Hz, 2H), 4.36-4.22 (m, 2H), 3.33-3.22 (m, 2H), 3.18 (s, 1H), 3.00 (dt, J=5.6, 9.2 Hz, 1H), 2.30-2.24 (m, 1H), 2.19-2.09 (m, 2H), 2.02-1.91 (m, 3H)

Step B. 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol: To a solution of 7-bromo-8-fluoro-2-(((2R,7aS)-2-fluorohexahydro-1H-pyrrolizin-7a(5H)-yl)methoxy)-4-(2,2,2-trifluoroethoxy)quinazoline (2.00 g, 1.0 equiv) and 5-ethyl-6-fluoro-4-(4,4,5,5-tetramethyl-1,3,2-dioxaborolan-2-yl)naphthalen-2-ol (1.57 g, 1.2 equiv) in CPME (20 mL) was added Cs₂CO₃ (1.5 M, 8.3 mL, 3.0 equiv) and Ad₂nBup-Pd-G₃ (604 mg, 0.2 equiv). The mixture was stirred at 100° C. for 2 hours under nitrogen atmosphere. The mixture was diluted with water (15 mL) and extracted with EtOAc (20 mL×2). The organic layer was dried with anhydrous sodium sulfate, filtered and concentrated under vacuum. The residue was purified by column chromatography (SiO₂, petroleum ether/ethyl acetate=I/O to 1/1) and concentrated under vacuum to afford the title compound (1.50 g, 61% yield) as brown solid; ¹H NMR (400 MHz, CDCl₃-d) δ=7.89-7.83 (m, 1H), 7.58 (dd, J=5.6, 8.8 Hz, 1H), 7.34-7.28 (m, 1H), 7.25-7.18 (m, 2H), 6.92 (dd, J=2.4, 15.2 Hz, 1H), 5.44-5.19 (m, 1H), 5.02-4.84 (m, 2H), 4.40-4.30 (m, 2H), 3.36-3.23 (m, 2H), 3.07-2.96 (m, 1H), 2.51-2.09 (m, 6H), 2.03-1.87 (m, 4H), 1.27 (t, J=6.8 Hz, 3H)

Step C. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)tetrahydropyrrolo[3,4-c]pyrrole-1,3(2H,3aH)-dione: To a solution of 2,3,3a,6a-tetrahydro-1H-pyrrolo[3,4-c]pyrrole-4,6-dione (35.5 mg, 2.0 equiv) in DMF (0.5 mL) and ACN (0.5 mL) was added K₃PO₄ (134 mg, 5.0 equiv) and stirred at 25° C. for 0.5 hour. Then 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol (75.0 mg, 1.0 equiv) was added into the reaction solution and stirred at 25° C. for 1.5 hours. The mixture was filtered to remove the insoluble and the filtrate was extracted by DCM (50 mL). The organic layer was concentrated in vacuum. The crude product was purified by prep-HPLC [Waters Xbridge 150×25 mm×5 um; A: water (NH₄HCO₃), B: ACN, B %: 40%-70% over 8 min] to afford the title compound (18.1 mg, 23% yield) as off-white solid; ¹H NMR (400 MHz, DMSO-d₆) δ=11.38 (br d, J=5.6 Hz, 1H), 9.92 (s, 1H), 8.06 (d, J=8.8 Hz, 1H), 7.79-7.75 (m, 1H), 7.38-7.34 (m, 1H), 7.31 (br d, J=2.8 Hz, 2H), 6.94 (d, J=2.4 Hz, 1H), 5.36-5.21 (m, 1H), 4.43 (br d, J=11.2 Hz, 2H), 4.21-4.15 (m, 2H), 4.11-4.00 (m, 2H), 3.68-3.64 (m, 2H), 3.10 (br d, J=12.8 Hz, 2H), 3.02 (br s, 1H), 2.83 (br d, J=6.0 Hz, 1H), 2.37-2.33 (m, 2H), 2.19-2.00 (m, 4H), 1.79 (br d, J=3.6 Hz, 2H), 0.74-0.69 (m, 3H); LCMS (ESI, M+1): m/z=632.3.

### Example 4

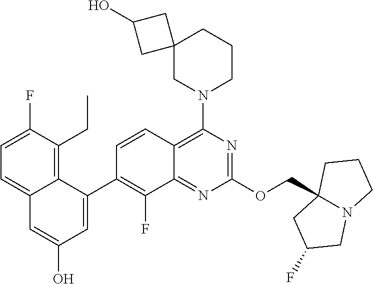 [SMILES: CCc1c(F)ccc2cc(O)cc(-c3ccc4c(N5CCCC6(CC(O)C6)C5)nc(OC[C@@]56CCCN5C[C@H](F)C6)nc4c3F)c12]

### 6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-azaspiro[3.5]nonan-2-ol

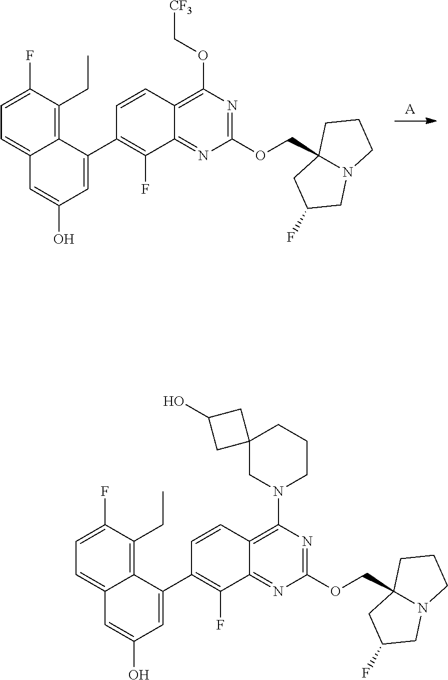

Step A. 6-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-6-azaspiro[3.5]nonan-2-ol: To a solution of 6-azaspiro[3.5]nonan-2-ol (45.0 mg, 3.0 equiv, HCl) in DMF (0.5 mL) was added K₃PO₄ (89.7 mg, 5.0 equiv). The mixture was stirred at 25° C. for 0.5 hour. Then 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol (50.0 mg, 1.0 equiv) was added. The mixture was stirred at 40° C. for 2 hours. The mixture was filtered to remove the insoluble. The crude product was purified by prep-HPLC [Phenomenex Synergi Polar-RP 100×25 mm×4 um; A: water (TFA), B: ACN, B %: 31%-51% over 7 min] to afford the title compound (4.19 mg, 7.6% yield) as white solid; ¹H NMR (400 MHz, DMSO-d₆) δ=10.17-9.60 (m, 1H), 7.94 (d, J=8.4 Hz, 1H), 7.77 (dd, J=6.0, 9.2 Hz, 1H), 7.51-7.45 (m, 1H), 7.39-7.31 (m, 2H), 6.94 (d, J=2.4 Hz, 1H), 5.46 (td, J=6.8, 17.6 Hz, 1H), 5.36 (br s, 1H), 4.19-4.07 (m, 2H), 3.14-3.07 (m, 2H), 3.02 (s, 1H), 2.83 (br d, J=6.0 Hz, 1H), 2.76 (br d, J=10.8 Hz, 2H), 2.69-2.65 (m, 2H), 2.36-2.32 (m, 2H), 2.12-1.94 (m, 6H), 1.89-1.73 (m, 4H), 1.68-1.60 (m, 2H), 1.51-1.43 (m, 2H), 0.70 (br t, J=7.2 Hz, 3H); LCMS (ESI, M+1): m/z=633.5.

### Example 5

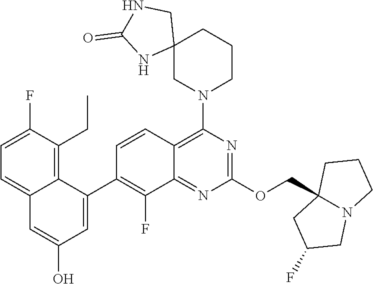 [SMILES: CCc1c(F)ccc2cc(O)cc(-c3ccc4c(N5CCCC6(CNC(=O)N6)C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decan-2-one

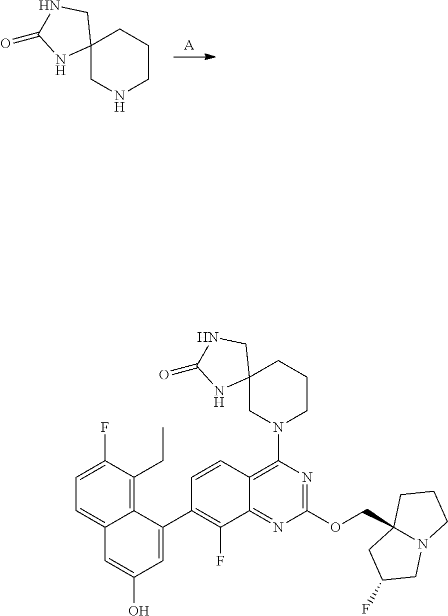

Step A. 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)guinazolin-4-yl)-1,3,7-triazaspiro[4.5]decan-2-one: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol(50.0 mg, 1.0 equiv) and 1,3,7-triazaspiro[4.5]decan-2-one (15.7 mg, 1.2 equiv) in DMF (0.1 mL) and ACN (0.1 mL) was added K₃PO₄ (53.8 mg, 3.0 equiv). The mixture was stirred at 60° C. for 12 hours. The mixture was diluted with water (2 mL) and extracted with EtOAc (2 mL×3). The organic layer was dried with anhydrous sodium sulfate, filtered and concentrated under vacuum. The residue was purified by prep-HPLC [Unisil 3-100 C18 Ultra 150×50 mm×3 um; A: water (FA), B: ACN, B %: 15%-45% over 7 min] and lyophilized. The impurity was further purified by prep-HPLC [Waters Xbridge 150×25 mm×5 um; A: water (NH₄HCO₃), B: ACN, B %: 37%-67% over 8 min] and lyophilized to afford the title compound (3.25 mg, 5.8% yield) as white solid; ¹H NMR (400 MHz, METHANOL-d₄) δ=7.89 (dd, J=2.8, 8.8 Hz, 1H), 7.66 (dd, J=6.0, 9.2 Hz, 1H), 7.37-7.30 (m, 1H), 7.27-7.18 (m, 2H), 6.95 (d, J=2.4 Hz, 1H), 5.51-5.27 (m, 1H), 4.50-4.27 (m, 2H), 4.02-3.81 (m, 4H), 3.42 (ddd, J=2.8, 9.2, 14.0 Hz, 4H), 3.30-3.27 (m, 1H), 3.14 (br dd, J=1.6, 6.4 Hz, 1H), 2.53-2.29 (m, 4H), 2.28-2.17 (m, 1H), 2.13-1.86 (m, 7H), 0.82-0.72 (m, 3H); LCMS (ESI, M+1): m/z=647.4.

### Example 6

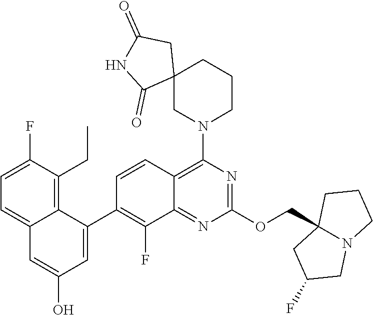 [SMILES: CCc1c(F)ccc2cc(O)cc(-c3ccc4c(N5CCCC6(CC(=O)NC6=O)C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-2,7-diazaspiro[4.5]decane-1,3-dione

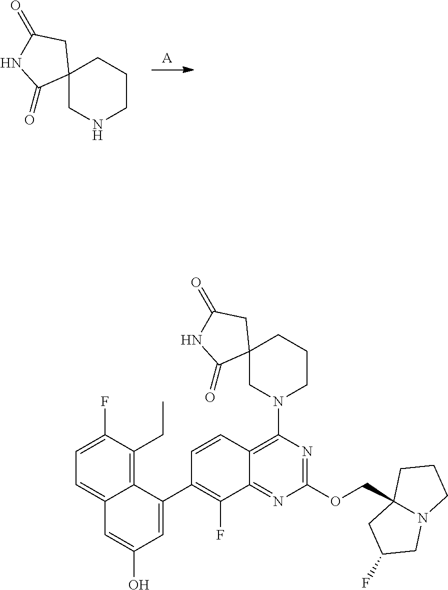

Step A. 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-2,7-diazaspiro[4.5]decane-1,3-dione: To a solution of 5-ethyl-6-fluoro-4-(8-fluoro-2-(((2R,7aS)-2-fluorohexahydro-1H-pyrrolizin-7a-yl)methoxy)-4-(2,2,2-trifluoroethoxy)quinazolin-7-yl)naphthalen-2-ol (70.0 mg, 1.0 equiv) and 2,7-diazaspiro[4.5]decane-1,3-dione (36.3 mg, 1.8 equiv) in DMF (0.1 mL) and ACN (0.1 mL) was added K₃PO₄ (75.4 mg, 3.0 equiv). The mixture was stirred at 40° C. for 12 hours. The mixture was quenched by addition of water (2 mL) and then extracted with ethyl acetate (3×5 mL). The combined organic layers were dried over anhydrous sodium sulfate, concentrated to give a residue. The residue was purified by prep-HPLC [Waters Xbridge 150×25 mm×5 μm; A: water (10 mM NH₄HCO₃), B: ACN, B %: 41%-71% over 8 min] and lyophilized to afford the title compound (10.0 mg, 11% yield) as white solid; ¹H NMR (400 MHz, METHANOL-d₄) δ=7.83 (br d, J=8.4 Hz, 1H), 7.70-7.60 (m, 1H), 7.35-7.28 (m, 1H), 7.27-7.18 (m, 2H), 6.95 (s, 1H), 5.41-5.36 (m, 1H), 5.24 (br d, J=1.2 Hz, 1H), 4.63-4.47 (m, 1H), 4.43-4.33 (m, 1H), 4.30-4.17 (m, 2H), 3.71-3.54 (m, 2H), 3.23 (br s, 2H), 3.08-2.84 (m, 2H), 2.68 (dd, J=2.0, 18.0 Hz, 1H), 2.56 (br d, J=5.6 Hz, 3H), 2.29-2.11 (m, 3H), 2.06-1.85 (m, 6H), 1.30 (br dd, J=1.6, 3.2 Hz, 1H), 0.87-0.66 (m, 3H). LCMS (ESI, M+1): m/z=660.4.

### Example 7

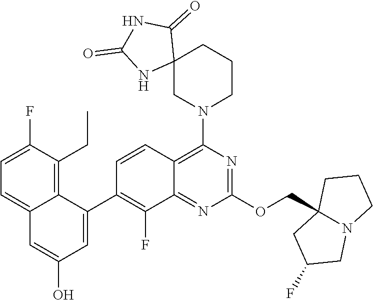 [SMILES: CCc1c(F)ccc2cc(O)cc(-c3ccc4c(N5CCCC6(C5)NC(=O)NC6=O)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione

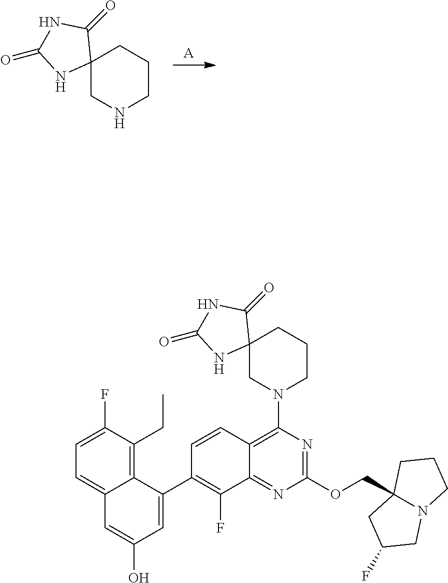

Step A. 7-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol (150 mg, 1.0 equiv) and 1,3,7-triazaspiro[4.5]decane-2,4-dione (51.5 mg, 1.2 equiv) in DMF (0.5 mL) and ACN (0.5 mL) was added K₃PO₄ (161 mg, 3.0 equiv). The mixture was stirred at 40° C. for 12 hours. The mixture was diluted with water (2 mL) and extracted with EtOAc (2 mL×3). The combined organic layers were dried over anhydrous sodium sulfate and concentrated under vacuum. The residue was purified by prep-HPLC [Phenomenex Synergi C18 150×25 mm×10 um; A: water (FA), B: ACN, B %: 15%-45% over 10 min] twice to afford the title compound (8.08 mg, 4.5% yield, FA) as yellow solid; ¹H NMR (400 MHz, DMSO-d₆) δ=10.83 (br d, J=1.2 Hz, 1H), 8.70 (d, J=7.6 Hz, 1H), 8.18 (br s, 1H), 7.84 (br dd, J=3.6, 8.4 Hz, 1H), 7.76 (dd, J=6.0, 9.2 Hz, 1H), 7.39-7.27 (m, 3H), 6.96-6.90 (m, 1H), 5.40-5.16 (m, 1H), 4.30-4.17 (m, 2H), 4.14-4.07 (m, 1H), 4.05-3.98 (m, 1H), 3.46 (br d, J=13.2 Hz, 2H), 3.36-3.33 (m, 1H), 3.09 (br d, J=12.4 Hz, 2H), 2.86-2.78 (m, 1H), 2.38-2.31 (m, 2H), 2.15-1.97 (m, 5H), 1.91-1.74 (m, 5H), 0.71 (br t, J=7.2 Hz, 3H); LCMS (ESI, M+1): m/z=661.3.

### Example 8

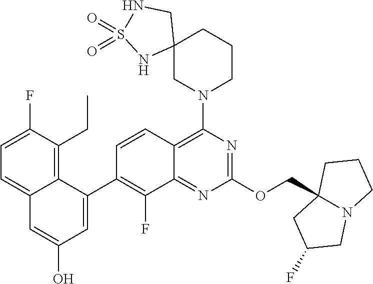 [SMILES: CCc1c(F)ccc2cc(O)cc(-c3ccc4c(N5CCCC6(CNS(=O)(=O)N6)C5)nc(OC[C@@]56CCCN5C[C@H](F)C6)nc4c3F)c12]

### 7-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-2-thia-1,3,7-triazaspiro[4.5]decane 2,2-dioxide

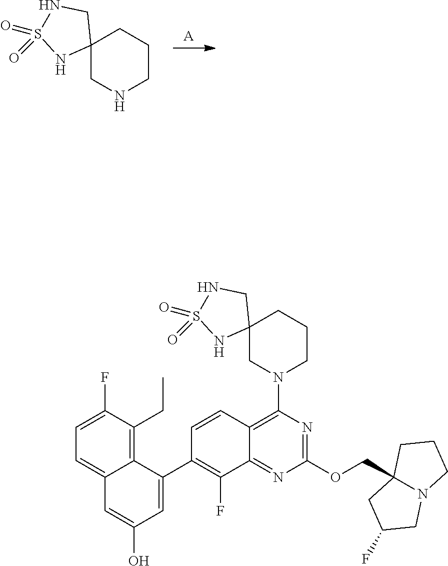

Step A. 7-(7-(8-ethyl-7-fluoro-3-hydroxynaphthalen-1-yl)-8-fluoro-2-(((2R,7aS)-2-fluorohexahydro-1H-pyrrolizin-7a(5H)-yl)methoxy)quinazolin-4-yl)-2-thia-1,3,7-triazaspiro[4.5]decane 2,2-dioxide: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol(200 mg, 1.0 equiv) and 2-thia-1,3,7-triazaspiro[4.5]decane 2,2-dioxide (70.0 mg, 1.1 equiv) in DMF (0.5 mL) and ACN (0.5 mL) was added K₃PO₄ (215 mg, 3.0 equiv). The mixture was stirred at 40° C. for 12 hours. The mixture was diluted with water (2 mL) and extracted with EtOAc (2 mL×3). The organic layer was dried over anhydrous sodium sulfate and concentrated under vacuum. The residue was purified by prep-HPLC [Phenomenex Synergi C18 150×25 mm×10 um; A: water (FA), B: ACN, B %: 16%-46% over 10 min] twice to afford the title compound (16.39 mg, 6.7% yield) as light yellow solid; ¹H NMR (400 MHz, DMSO-d₆) δ=9.92 (br s, 1H), 7.85 (dd, J=8.8, 13.6 Hz, 1H), 7.76 (dd, J=6.4, 8.4 Hz, 1H), 7.38-7.26 (m, 4H), 7.24-7.13 (m, 1H), 6.92 (s, 1H), 5.43-5.12 (m, 1H), 4.15-3.93 (m, 3H), 3.87-3.75 (m, 1H), 3.73-3.43 (m, 2H), 3.32-3.27 (m, 1H), 3.14-3.06 (m, 3H), 3.01 (br s, 1H), 2.88-2.77 (m, 1H), 2.42-2.31 (m, 2H), 2.13 (br s, 1H), 2.08-1.96 (m, 3H), 1.90-1.74 (m, 6H), 0.71 (dt, J=2.4, 7.2 Hz, 3H); LCMS (ESI, M+1): m/z=683.3.

### Example 9

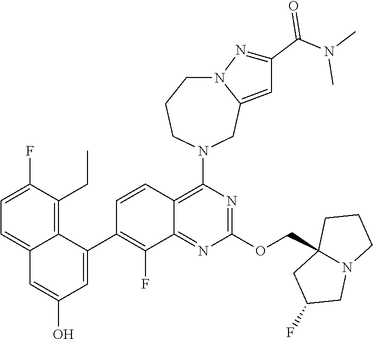 [SMILES: CCc1c(F)ccc2cc(O)cc(-c3ccc4c(N5CCCn6nc(C(=O)N(C)C)cc6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

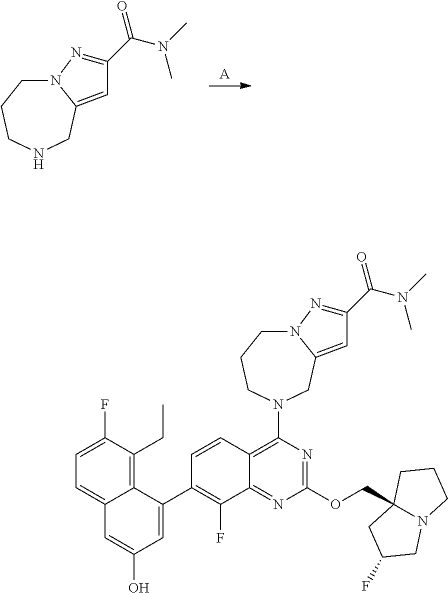

Step A. 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol(55.0 mg, 1.0 equiv) and N,N-dimethyl-5,6,7,8-tetrahydro-4H-pyrazolo[1,5-a][1,4]diazepine-2-carboxamide (23.2 mg, 1.2 equiv) in DMF (0.1 mL) and ACN(0.1 mL) was added K₃PO₄ (59.2 mg, 3.0 equiv). The mixture was stirred at 60° C. for 12 hours. The mixture was diluted with water (2 mL) and extracted with EtOAc (2 mL×3). The organic layers were dried with anhydrous sodium sulfate and concentrated under vacuum. The residue was purified by prep-HPLC [Unisil 3-100 C18 Ultra 150×50 mm×3 um; A: water (FA), B: ACN, B %: 17%-47% over 7 min] twice to afford the title compound (3.80 mg, 5.7% yield) as white solid; ¹H NMR (400 MHz, METHANOL-d₄) δ=7.95 (d, J=8.4 Hz, 1H), 7.65 (dd, J=5.6, 9.2 Hz, 1H), 7.35 (t, J=8.0 Hz, 1H), 7.27-7.19 (m, 2H), 6.95 (d, J=2.4 Hz, 1H), 6.71 (s, 1H), 5.45-5.27 (m, 1H), 5.24-5.10 (m, 2H), 4.56-4.50 (m, 2H), 4.45-4.24 (m, 4H), 3.52-3.33 (m, 6H), 3.14-3.06 (m, 4H), 2.49-2.31 (m, 5H), 2.29-2.14 (m, 2H), 2.12-1.89 (m, 3H), 0.76 (t, J=6.8 Hz, 3H); LCMS (ESI, M+1): m/z=700.3.

### Example 10

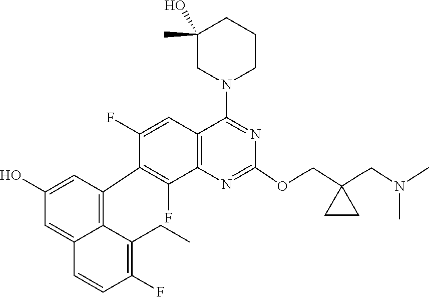 [SMILES: CCc1c(F)ccc2cc(O)cc(-c3c(F)cc4c(N5CCC[C@@](C)(O)C5)nc(OCCC5(CCN(C)C)CC5)nc4c3F)c12]

### (3R)-1-(2-((1-((dimethylamino)methyl)cyclopropyl)methoxy)-7-(8-ethyl-7-fluoro-3-hydroxynaphthalen-1-yl)-6,8-difluoroquinazolin-4-yl)-3-methylpiperidin-3-ol

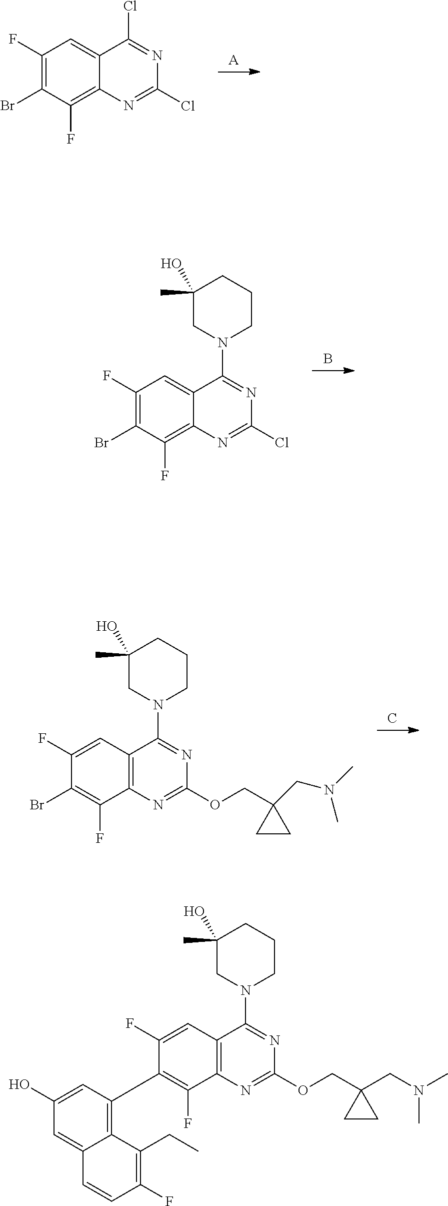

Step A. (R)-1-(7-bromo-2-chloro-6,8-difluoroquinazolin-4-yl)-3-methylpiperidin-3-ol: To a solution of 7-bromo-2,4-dichloro-6,8-difluoro-quinazoline (300 mg, 1 equiv) and 4 Å MS (1.5 mg) in DCM (0.5 mL) was added DIEA (370 mg, 3 equiv) and (R)-3-methylpiperidin-3-ol (132 mg, 1.2 equiv). The mixture was stirred at 0° C. for 0.5 hr. After completion, the residue was extracted with DCM (10 mL×3). The combined organic layers were dried over Na₂SO₄ and concentrated under reduced pressure to give a residue. The residue was purified by reversed-phase HPLC(0.1% FA condition) to afford the title compound (170 mg, 45% yield) as yellow solid. LCMS (ESI, M+1, M+3): m/z=391.9, 393.9.

Step B. (R)-1-(7-bromo-2-((1-((dimethylamino)methyl)cyclopropyl)methoxy)-6,8-difluoroquinazolin-4-yl)-3-methylpiperidin-3-ol: To a solution of (3R)-1-(7-bromo-2-chloro-6,8-difluoro-quinazolin-4-yl)-3-methyl-piperidin-3-ol (165 mg, 1 equiv) and 4 Å MS (1.5 mg) in dioxane (1 mL) was added DIEA (217 mg, 4 equiv) and (1-((dimethylamino)methyl)cyclopropyl)methanol (271 mg, 5 equiv). The mixture was stirred at 90° C. for 48 hrs. After completion, the reaction mixture was filtered and purified by reversed-phase HPLC (0.1% FA condition) to afford the title compound (67 mg, 33% yield) as yellow oil. LCMS (ESI, M+1, M+3): m/z=484.9, 486.9.

Step C. (3R)-1-(2-((1-((dimethylamino)methyl)cyclopropyl)methoxy)-7-(8-ethyl-7-fluoro-3-hydroxynaphthalen-1-yl)-6,8-difluoroquinazolin-4-yl)-3-methylpiperidin-3-ol: A mixture of (R)-1-(7-bromo-2-((1-((dimethylamino)methyl)cyclopropyl)methoxy)-6,8-difluoroquinazolin-4-yl)-3-methylpiperidin-3-ol (62 mg, 1 equiv), 5-ethyl-6-fluoro-4-(4,4,5,5-tetramethyl-1,3,2-dioxaborolan-2-yl)naphthalen-2-ol (69.3 mg, 2 equiv), methanesulfonato(diadamantyl-n-butylphosphino)-2-amino-1,1-biphenyl-2-yl)palladium(II) (7.98 mg, 0.1 equiv) and Cs₂CO₃ (107=mg, 3 equiv) in THE (1 mL) was degassed and purged with N₂ for 3 times, and then the mixture was stirred at 60° C. for 12 hours under N₂ atmosphere. After completion, the mixture was extracted with EtOAc (10 mL×3). The combined organic layers were dried over Na₂SO₄ and concentrated under reduced pressure to give a residue. The residue was purified by prep-HPLC (column: Phenomenex Luna C18 100×30 mm×5 μm; mobile phase: [water(FA)−ACN];B %: 17%-47%,8 min) to afford the title compound (18.4 mg, 26% yield) as white solid. ¹H NMR (400 MHz, METHANOL-d4) δ=7.88-7.77 (m, 1H), 7.68 (dd, J 6.0, 9.2 Hz, 1H), 7.33-7.20 (m, 2H), 6.98 (d, J 2.4 Hz, 1H), 4.43-4.34 (m, 2H), 4.32-4.21 (m, 1H), 4.08 (br d, J 13.6 Hz, 1H), 3.54-3.42 (m, 1H), 3.42-3.35 (m, 1H), 2.86 (br s, 2H), 2.69-2.50 (m, 7H), 2.49-2.41 (m, 1H), 2.20-2.09 (m, 1H), 1.88-1.71 (m, 3H), 1.28 (d, J 11.2 Hz, 3H), 0.88-0.76 (m, 5H), 0.73-0.61 (m, 2H); LCMS (ESI, M+1): m/z=595.3.

### Example 11

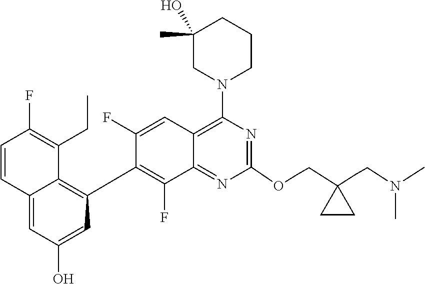

### (R)-1-(2-((1-((dimethylamino)methyl)cyclopropyl)methoxy)-7-((R)-8-ethyl-7-fluoro-3-hydroxynaphthalen-1-yl)-6,8-difluoroquinazolin-4-yl)-3-methylpiperidin-3-ol

### Example 12

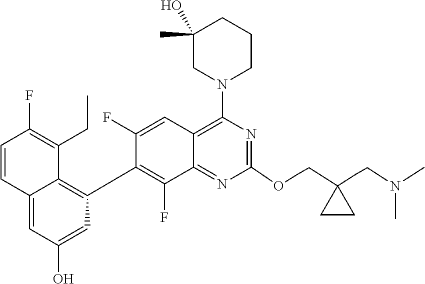

### (R)-1-(2-((1-((dimethylamino)methyl)cyclopropyl)methoxy)-7-((R)-8-ethyl-7-fluoro-3-hydroxynaphthalen-1-yl)-6,8-difluoroquinazolin-4-yl)-3-methylpiperidin-3-ol

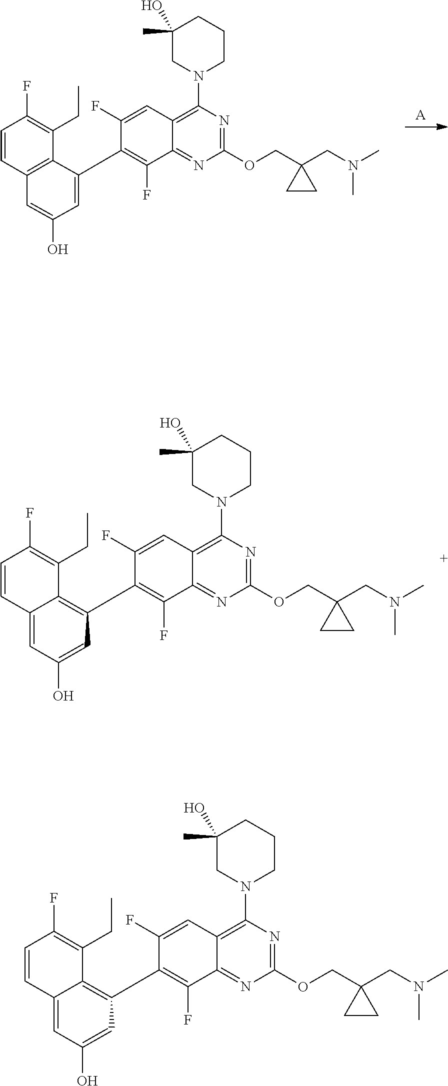

Step A. (R)-1-(2-((1-((dimethylamino)methyl)cyclopropyl)methoxy)-7-((R)-8-ethyl-7-fluoro-3-hydroxynaphthalen-1-yl)-6,8-difluoroquinazolin-4-yl)-3-methylpiperidin-3-ol: (3R)-1-(2-((1-((dimethylamino)methyl)cyclopropyl)methoxy)-7-(8-ethyl-7-fluoro-3-hydroxynaphthalen-1-yl)-6,8-difluoroquinazolin-4-yl)-3-methylpiperidin-3-ol (35.0 mg, 1 equiv) was purified with SFC [column: DAICEL CHIRALPAK AD 250 mm×30 mm×10 μm; mobile phase: 0.1% NH₃·1H₂O in IPA; B %: 45%-45%, 3.3 minutes](t_{R}: 2.232 min) and prep-HPLC [column: Phenomenex luna C18 150×25 mm×10 μm; mobile phase: water(FA)−ACN]; B %: 20%-50%, 2 minutes] to afford two peaks.

Peak 1 (Example 11) (35.0 mg, 1 equiv) was purified by SFC [column: DAICEL CHIRALPAK AD (250 mm×30 mm×10 m); mobile phase: 0.1% NH₃·H₂O in IPA; B %: 45%-45%, 3.3 minutes](t_{R}: 1.944 min) and prep-HPLC [column: Phenomenex luna C18 150×25 mm×10 μm; mobile phase: water(FA)−ACN]; B %: 20%-50%, 2 minutes] to afford the tittle compound (6.74 mg, 18% yield, 0.22 FA) as white solid; ¹H NMR (400 MHz, methanol-d₄) δ=7.83 (dd, J=1.6, 10.0 Hz, 1H), 7.68 (dd, J=6.0, 9.2 Hz, 1H), 7.29 (d, J=2.8 Hz, 1H), 7.25 (t, J=9.2 Hz, 1H), 6.98 (d, J=2.8 Hz, 1H), 4.38 (s, 2H), 4.24 (br d, J=13.6 Hz, 1H), 4.07 (br d, J=13.2 Hz, 1H), 3.48-3.34 (m, 2H), 2.79 (br s, 2H), 2.57 (br s, 6H), 2.53-2.31 (m, 2H), 2.24-2.07 (m, 1H), 1.91-1.69 (m, 3H), 1.29 (s, 3H), 0.90-0.75 (m, 5H), 0.65 (br s, 2H); LCMS (ESI, M+1): m/z=595.4; HPLC: >99% ee, column: Chiralpak AD-3 50×4.6 mm I.D., 3 μm, mobile phase: phase A for CO₂, and phase B for IPA (0.05% DEA), gradient elution: IPA (0.05% DEA) in CO₂ from 5% to 40%, flow rate: 3 mL/min, detector: 220 nm, t_{R}: 1.942 min.

Peak 2 (Example 12) (13.5 mg, 36% yield, 0.4 FA) as white solid; ¹H NMR (400 MHz, methanol-d₄) δ=7.80 (br d, J=9.6 Hz, 1H), 7.68 (dd, J=6.0, 9.2 Hz, 1H), 7.29 (d, J=2.8 Hz, 1H), 7.25 (t, J=9.6 Hz, 1H), 6.98 (d, J=2.8 Hz, 1H), 4.44-4.33 (m, 2H), 4.27 (br d, J=12.8 Hz, 1H), 4.07 (br d, J=13.2 Hz, 1H), 3.51 (d, J=13.2 Hz, 1H), 3.45-3.35 (m, 1H), 2.89-2.70 (m, 2H), 2.67-2.53 (m, 6H), 2.53-2.32 (m, 2H), 2.23-2.08 (m, 1H), 1.91-1.68 (m, 3H), 1.26 (s, 3H), 0.90-0.74 (m, 5H), 0.66 (br s, 2H); LCMS (ESI, M+1): m/z=595.4; HPLC: 99% ee, column: Chiralpak AD-3 50×4.6 mm I.D., 3 μm, mobile phase: phase A for CO₂, and phase B for IPA (0.05% DEA), gradient elution: IPA (0.05% DEA) in CO₂ from 5% to 40%, flow rate: 3 mL/min, detector: 220 nm, t_{R}: 2.227 min.

### Example 13

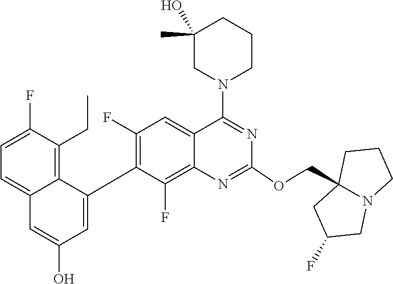 [SMILES: CCc1c(F)ccc2cc(O)cc(-c3c(F)cc4c(N5CCC[C@@](C)(O)C5)nc(OCC[C@@]56CCCN5C[C@H](F)C6)nc4c3F)c12]

### (3R)-1-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-methylpiperidin-3-ol

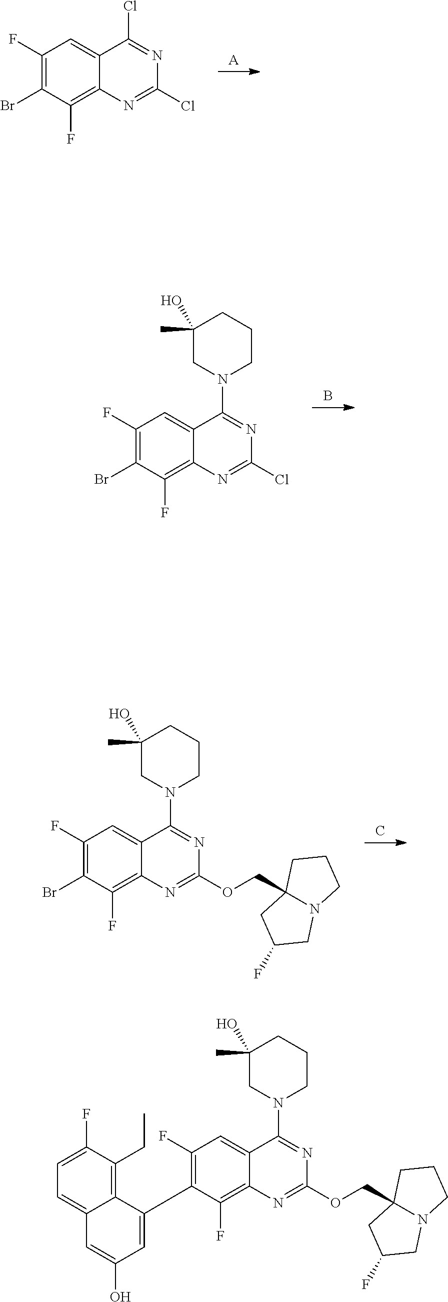

Step A. (R)-1-(7-bromo-2-chloro-6,8-difluoroquinazolin-4-yl)-3-methylpiperidin-3-ol: To a mixture of 7-bromo-2,4-dichloro-6,8-difluoroquinazoline (2 g, 1.0 equiv), 4 Å MS (600 mg) and DIPEA (3.29 g, 4.4 mL, 4.0 equiv) in DCM (20 mL) was added (R)-3-methylpiperidin-3-ol (660 mg, 0.9 equiv) in DCM (3 mL) dropwise at 0° C. The mixture was stirred at 25° C. for 0.5 hour. The mixture was filtered and diluted with water (30 mL), extracted with DCM (3×30 mL), washed with brine (50 mL), dried over Na₂SO₄, and concentrated. The residue was purified by reversed phase flash [C18, 0.1% formic acid condition] to afford the title compound (1.4 g, 55% yield) as yellow solid; LCMS [ESI, M+3]: m/z=393.7.

Step B. (R)-1-(7-bromo-6,8-difluoro-2-(((2R,7aS)-2-fluorohexahydro-1H-pyrrolizin-7a-yl)methoxy)guinazolin-4-yl)-3-methylpiperidin-3-ol: To a solution of (R)-1-(7-bromo-2-chloro-6,8-difluoroquinazolin-4-yl)-3-methylpiperidin-3-ol (230 mg, 1.0 equiv) and ((2R,7aS)-2-fluorohexahydro-1H-pyrrolizin-7a-yl)methanol (102 mg, 1.1 equiv) in dioxane (2 mL) were added DIPEA (227 mg, 306 μL, 1.1 equiv) and 4 Å MS (50 mg). The mixture was stirred at 100° C. for 72 hours. The mixture was diluted with H₂O (5 mL) and extracted with ethyl acetate (4×5 mL). The combined organic layers were dried over anhydrous Na₂SO₄, and concentrated. The residue was purified by reversed phase flash [C18, 0.1% formic acid condition] to afford the title compound (86 mg, 27% yield) as yellow solid; LCMS [ESI, M+3]: m/z=517.2.

Step C. (3R)-1-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-methylpiperidin-3-ol: A mixture of (R)-1-(7-bromo-6,8-difluoro-2-(((2R,7aS)-2-fluorohexahydro-1H-pyrrolizin-7a-yl)methoxy)quinazolin-4-yl)-3-methylpiperidin-3-ol (70.0 mg, 1.0 equiv), 5-ethyl-6-fluoro-4-(4,4,5,5-tetramethyl-1,3,2-dioxaborolan-2-yl)naphthalen-2-ol (85.9 mg, 2.0 equiv) and Ad₂nBuP-Pd-G3 (9.89 mg, 0.1 equiv), Cs₂CO₃ (1.5 M, 272 μL, 3.0 equiv) in THF (1 mL) was degassed and purged with N₂ for 3 times, and then the mixture was stirred at 60° C. for 2 hours under N₂ atmosphere. The mixture was filtered, diluted with water (10 mL) and extracted with ethyl acetate (4×10 mL). The combined organic layers were dried over anhydrous Na₂SO₄, concentrated. The residue was purified by reversed phase flash [C18, 0.1% formic acid condition] and prep-HPLC [Waters Xbridge 150×25 mm×5 μm; A: water (NH₄HCO₃), B: ACN; B %: 50%-80% over 10 minutes] to afford the title compound (11 mg, 12% yield) as white solid; ¹H NMR (400 MHz, METHANOL-d₄) δ=7.80-7.77 (m, 1H), 7.67 (dd, J=5.6, 9.2 Hz, 1H), 7.31-7.21 (m, 2H), 6.98 (d, J=2.4 Hz, 1H), 5.39-5.23 (m 1H), 4.63-4.59 (m, 1H), 4.35-4.18 (m, 3H), 4.05 (br d, J=13.2 Hz, 1H), 3.55-3.37 (m, 2H), 3.24-3.17 (m, 2H), 3.08-2.95 (m, 1H), 2.62-2.50 (m, 1H), 2.49-2.26 (m, 2H), 2.25-2.10 (m, 3H), 2.03-1.70 (m, 6H), 1.27 (d, J=12.0 Hz, 3H), 0.81 (q, J=7.2 Hz, 3H); SFC [Column: Chiralpak AD-3 50×4.6 mm ID, 3 μm Mobile phase: Phase A for CO₂, and Phase B for IPA (0.05% DEA); Gradient elution IPA (0.05% DEA) in CO₂ from 5% to 40%, Flow rate: 3 mL/min; Detector: PDA; Column Temp: 35C; Back Pressure: 100 Bar]; LCMS (ESI, M+1): m/z=625.3.

### Example 14

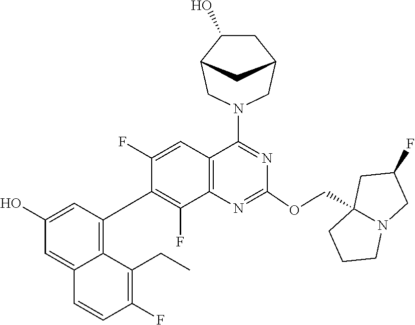 [SMILES: CCc1c(F)ccc2cc(O)cc(-c3c(F)cc4c(N5C[C@@H]6C[C@H](C5)[C@H](O)C6)nc(OCC[C@@]56CCCN5C[C@H](F)C6)nc4c3F)c12]

### (1R,5R,6R)-3-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-azabicyclo[3.2.1]octan-6-ol

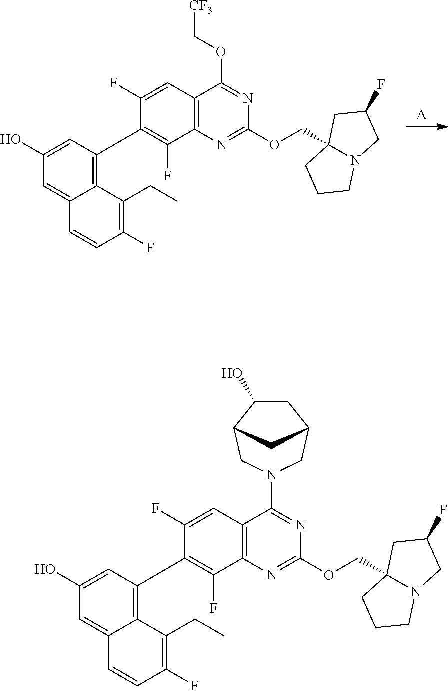

Step A. (1R,5R,6R)-3-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)guinazolin-4-yl)-3-azabicyclo[3.2.1]octan-6-ol: A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1.0 equiv), (1R,5R,6R)-3-azabicyclo[3.2.1]octan-6-ol (156 mg, 5.0 equiv) and DIEA (371 mg, 11.7 equiv), 4 Å molecular sieve (10 mg) in DMF (1 mL) was stirred at 40° C.-60° C. for 72 hours. After completion, the mixture was filtered and purified by prep-HPLC (column: Phenomenex luna C18 150*25 mm*10 um; mobile phase: [water(FA)−ACN];B %: 20%-50%,10 min) and (column: Welch Xtimate C18 150*25 mm*5 um; mobile phase: [water(NH₃H₂O)−ACN];B %: 45%-75%,8 min) to afford the title compound (7.57 mg, 4.8% yield) as white solid. ¹H NMR (400 MHz, METHANOL-d4) δ=8.04-7.96 (m, 1H), 7.69 (dd, J=5.6, 8.8 Hz, 1H), 7.33-7.20 (m, 2H), 6.99 (d, J=2.4 Hz, 1H), 5.44-5.22 (m, 1H), 4.77-4.67 (m, 1H), 4.61 (br d, J=11.2 Hz, 1H), 4.41-4.34 (m, 1H), 4.32 (d, J=10.4 Hz, 1H), 4.25-4.19 (m, 1H), 3.63-3.55 (m, 1H), 3.47 (br d, J=12.4 Hz, 1H), 3.40-3.35 (m, 1H), 3.30-3.14 (m, 3H), 3.09-2.99 (m, 1H), 2.66-2.53 (m, 1H), 2.48-2.41 (m, 1H), 2.38 (br d, J=4.0 Hz, 1H), 2.28 (br s, 1H), 2.27-2.24 (m, 1H), 2.24-2.14 (m, 2H), 2.08-1.97 (m, 2H), 1.96-1.81 (m, 3H), 1.60-1.45 (m, 1H), 0.87-0.78 (m, 3H). LCMS (ESI, M+1): m/z=637.3.

### Example 15

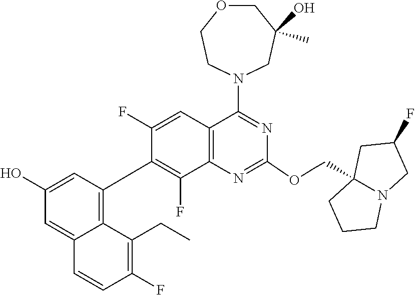 [SMILES: CCc1c(F)ccc2cc(O)cc(-c3c(F)cc4c(N5CCOC[C@@](C)(O)C5)nc(OCC[C@@]56CCCN5C[C@H](F)C6)nc4c3F)c12]

### (6S)-4-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol

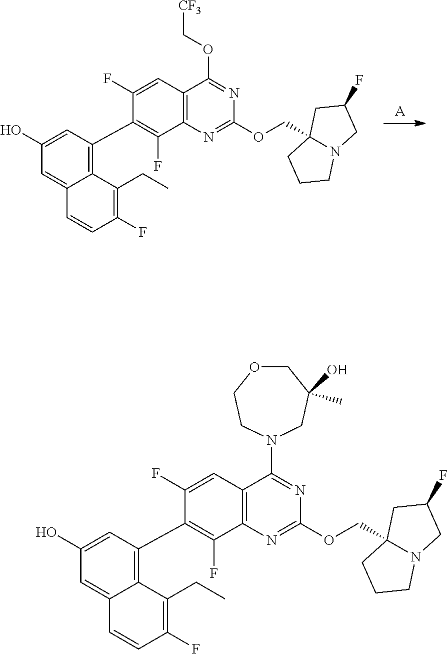

Step A. (6S)-4-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol: A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1.0 equiv), (S)-6-methyl-1,4-oxazepan-6-ol (226 mg, 7.0 equiv) and DIEA (191 mg, 6.0 equiv), 4 Å molecular sieve (10.0 mg) in DMF (1 mL) was stirred at 80° C. for 72 hrs. After completion, the mixture is filtered and purified by prep-HPLC (column: Phenomenex luna C18 150*25 mm*10 um; mobile phase: [water(FA)−ACN];B %: 20%-50%,10 min) to afford the title compound (16.1 mg, 9% yield) as off-white solid; ¹H NMR (400 MHz, METHANOL-d4) δ=8.25-8.07 (m, 1H), 7.68 (dd, J=6.0, 8.8 Hz, 1H), 7.31-7.21 (m, 2H), 6.98 (dd, J=2.4, 8.8 Hz, 1H), 5.54-5.32 (m, 1H), 4.55-4.37 (m, 4H), 4.13-3.97 (m, 2H), 3.94-3.82 (m, 2H), 3.75-3.56 (m, 3H), 3.50 (br dd, J=7.2, 9.6 Hz, 2H), 3.23-3.16 (m, 1H), 2.61-2.33 (m, 4H), 2.29-2.21 (m, 1H), 2.18-2.08 (m, 2H), 2.04-1.93 (m, 1H), 1.26 (d, J=8.4 Hz, 3H), 0.84-0.71 (m, 3H); LCMS (ESI, M+1): m/z=641.1.

### Example 16

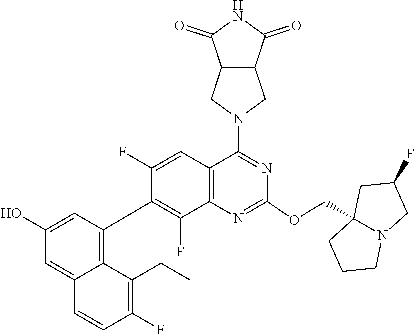 [SMILES: CCc1c(F)ccc2cc(O)cc(-c3c(F)cc4c(N5CC6C(=O)NC(=O)C6C5)nc(OCC[C@@]56CCCN5C[C@H](F)C6)nc4c3F)c12]

### 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)tetrahydropyrrolo[3,4-c]pyrrole-1,3(2H,3aH)-dione

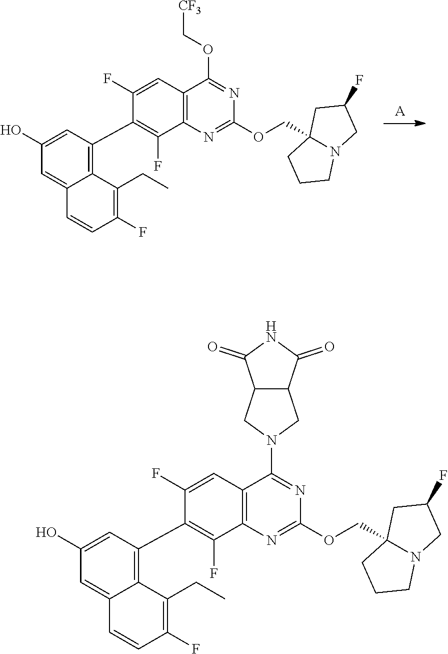

Step A. (3aR,6aS)-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)tetrahydropyrrolo[3,4-c]pyrrole-1,3(2H,3aH)-dione: A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (100 mg, 1.0 equiv), (3aR,6aS)-tetrahydropyrrolo[3,4-c]pyrrole-1,3(2H,3aH)-dione (161 mg, 7.0 equiv) and 4 Å molecular sieve (5 mg) in DIEA (1 mL) and DMF (0.5 mL) was stirred at 80° C. for 48 hrs. After completion, the mixture was filtered and purified by prep-HPLC (column: Phenomenex luna C18 150*25 mm*10 um; mobile phase: [water(NH₄HCO₃)−ACN];B %: 35%-65%,8 min) and prep-HPLC (column: Phenomenex luna C18 150*25 mm*10 um; mobile phase: [water(FA)−ACN];B %: 18%-48%,10 min). The desired fractions were collected and lyophilization. The residue was purified by prep-HPLC (column: Phenomenex luna C18 150*25 mm*10 um; mobile phase: [water(FA)−ACN];B %: 18%-48%,10 min) to afford the title compound (6.27 mg, 5% yield) as white solid; ¹H NMR (400 MHz, METHANOL-d4) δ=7.88 (br d, J=10.0 Hz, 1H), 7.68 (dd, J=6.0, 9.2 Hz, 1H), 7.34-7.18 (m, 2H), 6.99 (d, J=2.4 Hz, 1H), 5.53-5.33 (m, 1H), 4.66 (br d, J=12.0 Hz, 2H), 4.52-4.36 (m, 2H), 4.28-4.08 (m, 2H), 3.69 (br d, J=7.6 Hz, 2H), 3.73-3.45 (m, 2H), 3.26-3.18 (m, 1H), 2.59-2.33 (m, 4H), 2.31-2.24 (m, 1H), 2.22-2.11 (m, 2H), 2.08-1.99 (m, 1H), 0.79 (br t, J=6.8 Hz, 3H); LCMS (ESI, M+1): m/z=650.1.

### Example 17

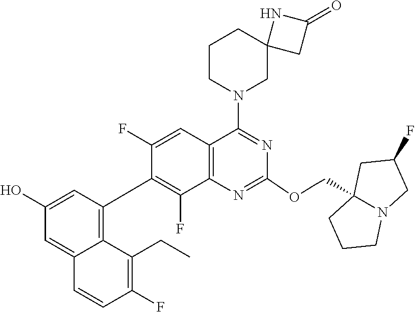 [SMILES: CCc1c(F)ccc2cc(O)cc(-c3c(F)cc4c(N5CCCC6(CC(=O)N6)C5)nc(OCC[C@@]56CCCN5C[C@H](F)C6)nc4c3F)c12]

### 6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,6-diazaspiro[3.5]nonan-2-one

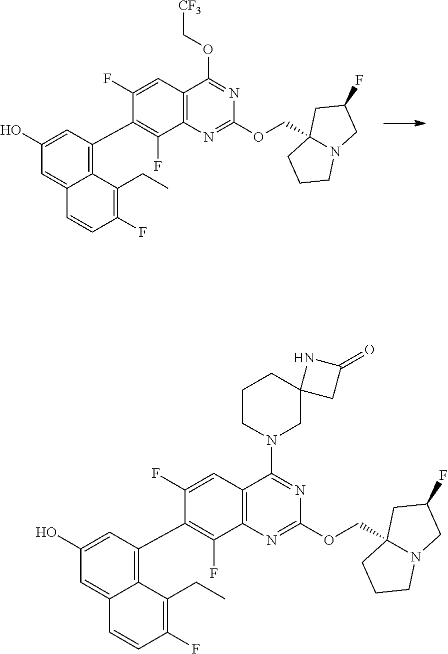 [SMILES: CCc1c(F)ccc2cc(O)cc(-c3c(F)cc4c(OCC(F)(F)F)nc(OC[C@@]56CCCN5C[C@H](F)C6)nc4c3F)c12 ; CCc1c(F)ccc2cc(O)cc(-c3c(F)cc4c(N5CCCC6(CC(=O)N6)C5)nc(OC[C@@]56CCCN5C[C@H](F)C6)nc4c3F)c12]

Step A. 6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,6-diazaspiro[3.5]nonan-2-one:

A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1 equiv) 1,6-diazaspiro[3.5]nonan-2-one (68.9 mg, 2 equiv), DIEA (191 mg, 6 equiv) in DMF (1 mL) was stirred 40° C. for 12 hours. After completion, the mixture was purified by prep-HPLC Phenomenex luna C18 150*25 mm*10 um; mobile phase: [water(FA)−ACN];B %: 20%-50%,10 min to afford the title compound (53.8 mg, 33% yield) as a white solid; ¹H NMR (400 MHz, METHANOL-d₄) δ=7.75-7.60 (m, 2H), 7.35-7.19 (m, 2H), 6.98 (s, 1H), 5.55-5.30 (m, 1H), 4.55-4.37 (m, 2H), 4.22-4.07 (m, 2H), 3.91-3.77 (m, 1H), 3.74-3.53 (m, 4H), 2.90-2.71 (m, 2H), 2.61-2.35 (m, 4H), 2.33-2.26 (m, 1H), 2.22-1.86 (m, 8H), 0.80 (br t, J=7.2 Hz, 3H). LCMS (ESI, M+1): m/z=650.6.

### Example 18

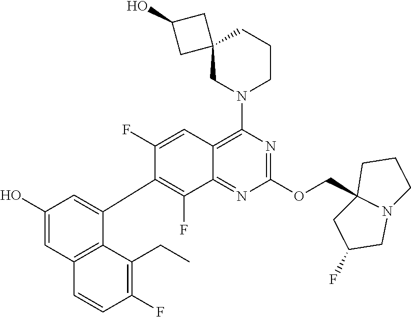 [SMILES: CCc1c(F)ccc2cc(O)cc(-c3c(F)cc4c(N5CCC[C@]6(C5)C[C@@H](O)C6)nc(OC[C@@]56CCCN5C[C@H](F)C6)nc4c3F)c12]

### (2S,4s)-6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-azaspiro[3.5]nonan-2-ol

### Example 19

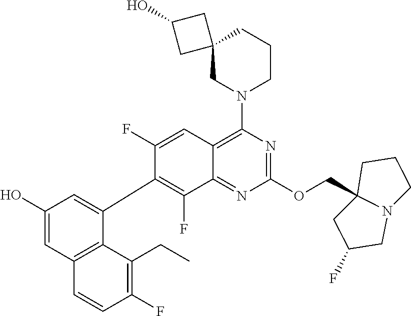 [SMILES: CCc1c(F)ccc2cc(O)cc(-c3c(F)cc4c(N5CCC[C@]6(C5)C[C@H](O)C6)nc(OC[C@@]56CCCN5C[C@H](F)C6)nc4c3F)c12]

### (2R,4r)-6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-azaspiro[3.5]nonan-2-ol

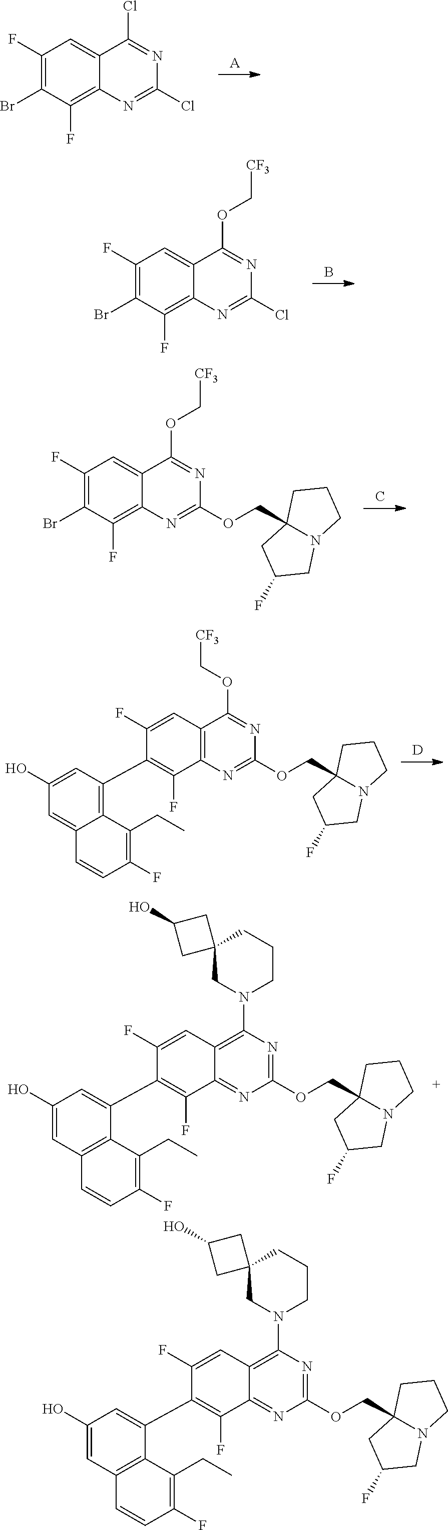

Step A. 7-bromo-2-chloro-6,8-difluoro-4-(2,2,2-trifluoroethoxy)quinazoline: To a solution of 2,2,2-trifluoroethanol (2.39 g, 1.0 equiv) in THF (80 mL) was added NaH (1.05 g, 1.1 equiv) at 0° C. After completion, the mixture was stirred at 10° C. for 0.5 hour and the mixture was added to a solution of 7-bromo-2,4-dichloro-6,8-difluoroquinazoline (7.5 g, 1.0 equiv) in THF (80 mL) with stirred at −40° C. The mixture was stirred at −40° C. for 1 hour and 25° C. for 2 hours. The reaction mixture was quenched by addition of H₂O (100 mL) at 0° C. and extracted with ethyl acetate (50 mL×2). The combined organic layers were washed with brine (50 mL), dried over Na₂SO₄, filtered and concentrated under reduced pressure to give a residue. The residue was purified by reversed phase flash [C18, water (FA, 0.1%)/acetonitrile] to afford the title compound (5 g, 54.3% yield) as yellow solid. LCMS (ESI, M+1, M+3): m/z=376.7, 378.7.

Step B. 7-bromo-6,8-difluoro-2-(((2R,7aS)-2-fluorohexahydro-1H-pyrrolizin-7a-yl)methoxy)-4-(2,2,2-trifluoroethoxy)quinazoline: To a mixture of 7-bromo-2-chloro-6,8-difluoro-4-(2,2,2-trifluoroethoxy)quinazoline (4.9 g, 1.0 equiv) and ((2R,7aS)-2-fluorohexahydro-1H-pyrrolizin-7a-yl)methanol (5.37 g, 2.6 equiv) in THE (50 mL) was added Na₂CO₃ (4.13 g, 3.0 equiv). The mixture was stirred at 40° C. for 24 hours and 60° C. for another 16 hours. After completion, the mixture was filtered to remove Na₂CO₃. The residue was diluted with water (100 ml) and extracted with ethyl acetate (50 ml×3). The combined organic layer was dried over Na₂SO₄, filtered and concentrated under vacuum to give a residue. The residue was purified by prep-HPLC (column: Phenomenex luna C18 250×50 mm×10 um; mobile phase: [water(FA)−ACN];B %: 25%-55%,30 min) to afford the title compound (3.3 g, 50% yield) as white solid. LCMS (ESI, M+1, M+3): m/z=499.9, 501.9.

Step C. 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol: To a solution of 7-bromo-6,8-difluoro-2-(((2R,7aS)-2-fluorohexahydro-1H-pyrrolizin-7a-yl)methoxy)-4-(2,2,2-trifluoroethoxy)quinazoline (3.3 g, 1.0 equiv) and 5-ethyl-6-fluoro-4-(4,4,5,5-tetramethyl-1,3,2-dioxaborolan-2-yl)naphthalen-2-ol (2.50 g, 1.2 equiv) in methoxycyclopentane (35 mL) was added Cs₂CO₃ (1.5 M, 3.0 equiv) at 25° C. The suspension was degassed under vacuum and purged with N₂ two times. Methanesulfonato(diadamantyl-n-butylphosphino)-2-amino-1,1-biphenyl-2-yl)palladium(II) (480 mg, 0.1 equiv) was added, the suspension was degassed under vacuum and purged with N₂ three times. The mixture was stirred at 90° C. for 2 hours. After completion, the mixture was diluted with water (30 mL) and extracted with ethyl acetate (30 mL×3). The combined organic layer was dried over Na₂SO₄, filtered and concentrated under vacuum to give a residue. The residue was purified by prep-HPLC (column: Phenomenex luna C18 250×50 mm×10 um; mobile phase: [water (FA)−ACN]; B %: 20%-50%,22 min) to afford the title compound (2.9 g, 71.4% yield) as white solid. LCMS (ESI, M+1): m/z=610.5.

Step D. (2S,4s)-6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-azaspiro[3.5]nonan-2-ol and Trans-(2R,4r)-6-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-azaspiro[3.5]nonan-2-ol: A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (100 mg, 1.0 equiv), 6-azaspiro[3.5]nonan-2-ol (43.7 mg, 1.5 equiv, HCl), 4 Å molecular sieve (50.0 mg, 1.0 equiv), K₃PO₄ (104 mg, 3.0 equiv) in DMF (0.5 mL) and ACN (0.5 mL) were degassed and purged with N₂ for 3 times, and then the mixture was stirred at 40° C. for 12 hours under N₂ atmosphere. After completion, the mixture was filtered. The reaction mixture was cooled to room temperature. Ethyl acetate (40 mL) and water (40 mL) were added, and layers were separated. The aqueous phase was extracted with ethyl acetate (2×30 mL). Combined extracts were washed with brine (40 mL), dried over sodium sulfate and filtered. The filtrate was concentrated under reduced pressure and was purified by prep-TLC (SiO₂, Dichloromethane/Methanol=10/1 column: Phenomenex Luna C18 150×25 mm×10 um; mobile phase: [water (FA)−ACN]; B %: 22%-52%, 10 min) to afford two peaks.

Example 18 (25.1 mg, 46.5% yield) as white solid: ¹H NMR (400 MHz, CD₃OD) δ=8.53 (br s, 1H), 7.68 (dd, J=6.0, 9.2 Hz, 1H), 7.63 (br d, J=10.0 Hz, 1H), 7.29 (d, J=2.4 Hz, 1H), 7.25 (t, J=9.2 Hz, 1H), 6.99 (d, J=2.4 Hz, 1H), 5.46-5.22 (m, 1H), 4.41-4.21 (m, 3H), 3.88 (s, 2H), 3.85-3.74 (m, 2H), 3.41 (br d, J=2.0 Hz, 3H), 3.14-2.99 (m, 1H), 2.66-2.51 (m, 1H), 2.48-2.25 (m, 3H), 2.25-2.14 (m, 3H), 2.11-1.99 (m, 2H), 1.98-1.90 (m, 1H), 1.89-1.68 (m, 6H), 0.81 (br t, J=7.2 Hz, 3H); LCMS (ESI, M+1): m/z=651.4.

Example 19 (10.3 mg, 18.7% yield) as white solid: ¹H NMR (400 MHz, METHANOL-d4) δ=8.54 (br s, 1H), 7.68 (dd, J=5.8, 9.2 Hz, 1H), 7.60 (br d, J=9.8 Hz, 1H), 7.30 (d, J=2.4 Hz, 1H), 7.25 (t, J=9.4 Hz, 1H), 6.99 (d, J=2.4 Hz, 1H), 5.54-5.20 (m, 1H), 4.43-4.29 (m, 2H), 4.24 (br t, J=7.3 Hz, 1H), 3.97-3.73 (m, 4H), 3.53-3.35 (m, 3H), 3.17-3.02 (m, 1H), 2.66-2.52 (m, 1H), 2.50-2.37 (m, 2H), 2.36-2.28 (m, 2H), 2.27-2.16 (m, 2H), 2.13-2.02 (m, 2H), 2.01-1.90 (m, 1H), 1.80 (br s, 4H), 1.69 (td, J=7.3, 11.7 Hz, 2H), 0.81 (br t, J=7.4 Hz, 3H); LCMS (ESI, M+1): m/z=651.4

### Example 20

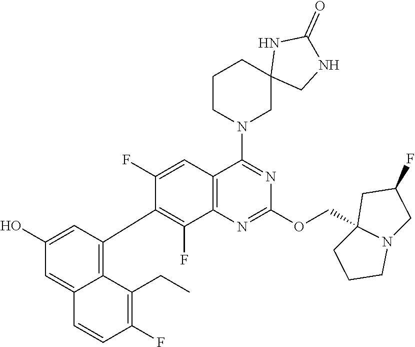 [SMILES: CCc1c(F)ccc2cc(O)cc(-c3c(F)cc4c(N5CCCC6(CNC(=O)N6)C5)nc(OC[C@@]56CCCN5C[C@H](F)C6)nc4c3F)c12]

### 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,3,7-triazaspiro[4.5]decan-2-one

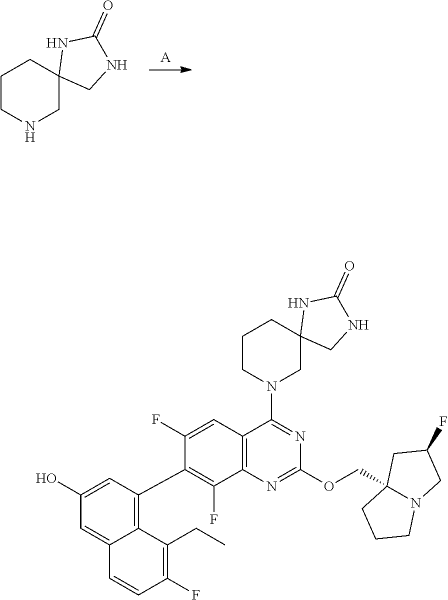

Step A. 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,3,7-triazaspiro[4.5]decan-2-one: To a mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1.0 equiv), 1,3,7-triazaspiro[4.5]decan-2-one (76.4 mg, 2.0 equiv) in DMF (0.2 mL) was added DIEA (318 mg, 10 equiv) and 4 Å molecular sieve (10 mg). The mixture was stirred at 60° C. for 12 hours. After completion, the mixture was filtered and purified by prep-HPLC (column: Phenomenex luna C18 150*25 mm*10 um; mobile phase: [water(FA)−ACN];B %: 18%-48%,10 min) and (column: Welch Xtimate C18 150*25 mm*5 um; mobile phase: [water(NH₃H₂O)−ACN];B %: 30%-60%,8 min) to afford the title compound (16.7 mg, 10% yield) as white solid. ¹H NMR (400 MHz, METHANOL-d4) δ=7.74-7.57 (m, 2H), 7.36-7.12 (m, 2H), 6.98 (br s, 1H), 5.44-5.20 (m, 1H), 4.37-4.17 (m, 2H), 3.95-3.73 (m, 4H), 3.43 (ddd, J=2.4, 6.8, 9.2 Hz, 1H), 3.30-3.13 (m, 4H), 3.06-2.95 (m, 1H), 2.60-2.11 (m, 4H), 2.06-1.84 (m, 7H), 0.88-0.71 (m, 3H). LCMS (ESI, M+1): m/z=665.2.

### Example 21

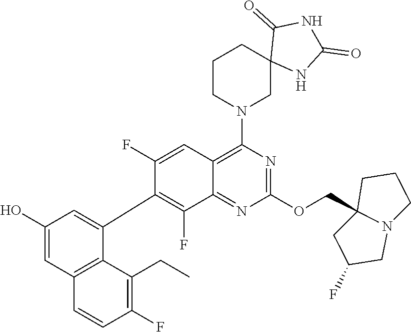 [SMILES: CCc1c(F)ccc2cc(O)cc(-c3c(F)cc4c(N5CCCC6(C5)NC(=O)NC6=O)nc(OC[C@@]56CCCN5C[C@H](F)C6)nc4c3F)c12]

### 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,3,7-triazaspiro[4.5]decane-2,4-dione

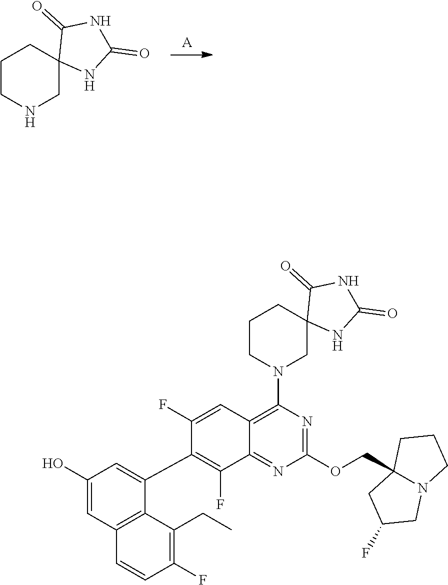

Step A. 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-1,3,7-triazaspiro[4.5]decane-2,4-dione: To a solution of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (100 mg, 1.0 equiv) and 1,3,9-triazaspiro[4.5]decane-2,4-dione (55.51 mg, 2.0 equiv) in DMF (0.5 mL) was added 4 Å MS (20 mg) and DIEA (63.6 mg, 3.0 equiv). The mixture was stirred at 40° C. for 24 hours. The reaction mixture was filtered and purified by prep-HPLC [column: Phenomenex C18 75×30 mm×3 μm; mobile phase: (water(FA)−ACN];B %: 18%-48%,7 min] to afford the title compound (32.7 mg, 29% yield) as white solid; ¹H NMR (400 MHz, METHANOL-d4) δ=7.77-7.66 (m, 2H), 7.36-7.22 (m, 2H), 7.01 (d, J=2.4 Hz, 1H), 5.56-5.35 (m, 1H), 4.56-4.31 (m, 4H), 3.74-3.49 (m, 5H), 3.30-3.21 (m, 1H), 2.64-2.37 (m, 4H), 2.35-2.15 (m, 4H), 2.12-1.94 (m, 4H), 0.88-0.76 (m, 3H)); LCMS (ESI, M+1): m/z=679.6.

### Example 22

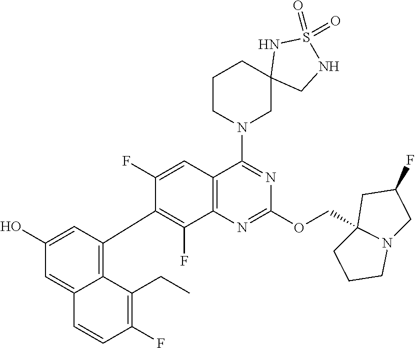 [SMILES: CCc1c(F)ccc2cc(O)cc(-c3c(F)cc4c(N5CCCC6(CNS(=O)(=O)N6)C5)nc(OC[C@@]56CCCN5C[C@H](F)C6)nc4c3F)c12]

### 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-2-thia-1,3,7-triazaspiro[4.5]decane 2,2-dioxide

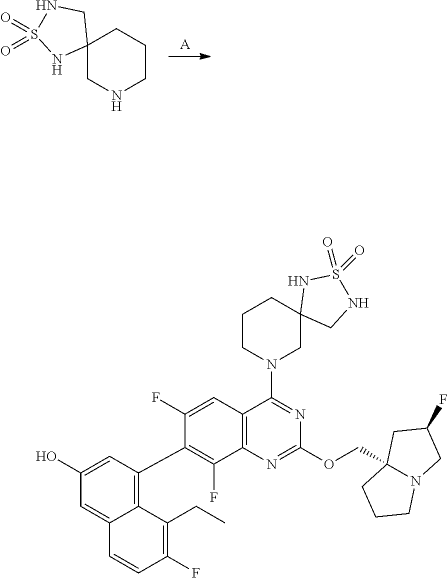

Step A. 7-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-2-thia-1,3,7-triazaspiro[4.5]decane 2,2-dioxide: To a solution of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1 equiv) in DMF (1.5 mL) were added DIEA (254 mg, 8 equiv), 4 Å molecular sieve (5 mg) and 2-thia-1,3,7-triazaspiro[4.5]decane 2,2-dioxide (94.1 mg, 2 equiv). The mixture was stirred at 40° C. for 72 hours. After completion, the mixture was filtered and purified by (column: Phenomenex luna C18 150×25 mm×10 μm; mobile phase: [water(FA)−ACN];B %: 20%-50%,10 min) to afford the title compound (9.9 mg, 5.4% yield) as yellow solid; ¹H NMR (400 MHz, METHANOL-d₄) δ=8.57-8.46 (m, 1H), 7.74-7.64 (m, 2H), 7.29 (d, J=2.8 Hz, 1H), 7.25 (t, J=9.2 Hz, 1H), 7.00-6.94 (m, 1H), 5.51-5.32 (m, 1H), 4.51-4.28 (m, 3H), 4.25-4.14 (m, 1H), 3.75-3.45 (m, 5H), 3.42 (br d, J=11.6 Hz, 1H), 3.25-3.17 (m, 2H), 2.63-2.33 (m, 4H), 2.32-2.22 (m, 1H), 2.17-1.98 (m, 5H), 1.95-1.81 (m, 2H), 0.81 (br t, J=7.2 Hz, 3H). LCMS (ESI, M+1): m/z=701.3.

### Example 23

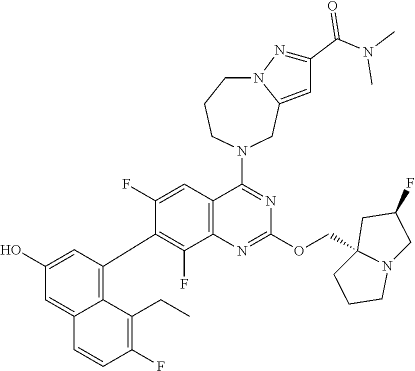 [SMILES: CCc1c(F)ccc2cc(O)cc(-c3c(F)cc4c(N5CCCn6nc(C(=O)N(C)C)cc6C5)nc(OC[C@@]56CCCN5C[C@H](F)C6)nc4c3F)c12]

### 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide

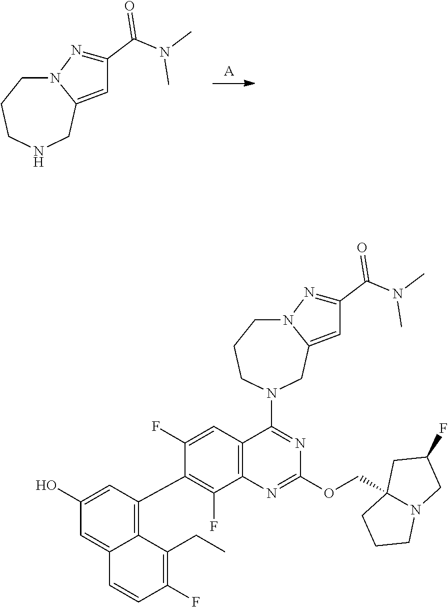

Step A. 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide: To a solution of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (150 mg, 1.0 equiv) in DMF (1.5 mL) was added DIEA (254 mg, 8.0 equiv), 4 Å molecular sieve (5 mg) and N,N-dimethyl-5,6,7,8-tetrahydro-4H-pyrazolo[1,5-a][1,4]diazepine-2-carboxamide (102 mg, 2.0 equiv). The mixture was stirred at 40° C. for 72 hrs. After completion, the mixture was filtered and purified by (column: Phenomenex luna C18 150×25 mm×10 μm; mobile phase: [water(FA)−ACN];B %: 22%-52%,10 min) and prep-HPLC (column: Welch Xtimate C18 150×25 mm×5 μm; mobile phase: [water(NH₃H₂O)−ACN];B %: 32%-62%,8 min) to afford the title compound (12.9 mg, 7.1% yield) as yellow solid; ¹H NMR (400 MHz, METHANOL-d₄) δ=7.84-7.63 (m, 2H), 7.35-7.20 (m, 2H), 6.98 (br s, 1H), 6.71 (s, 1H), 5.56-5.31 (m, 1H), 5.24-5.06 (m, 2H), 4.61-4.28 (m, 6H), 3.70-3.45 (m, 3H), 3.35 (s, 3H), 3.03 (s, 4H), 2.71-2.48 (m, 2H), 2.46-2.24 (m, 5H), 2.22-1.96 (m, 3H), 0.80 (br t, J=6.8 Hz, 3H); LCMS(ESI, M+1): m/z=718.3.

### Example 24

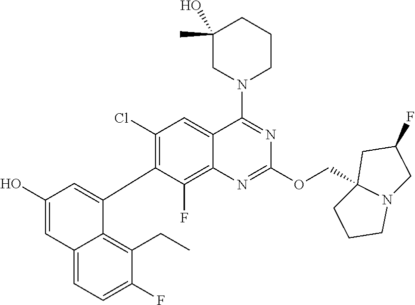 [SMILES: CCc1c(F)ccc2cc(O)cc(-c3c(Cl)cc4c(N5CCC[C@@](C)(O)C5)nc(OC[C@@]56CCCN5C[C@H](F)C6)nc4c3F)c12]

### (3R)-1-(6-chloro-7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-3-methylpiperidin-3-ol

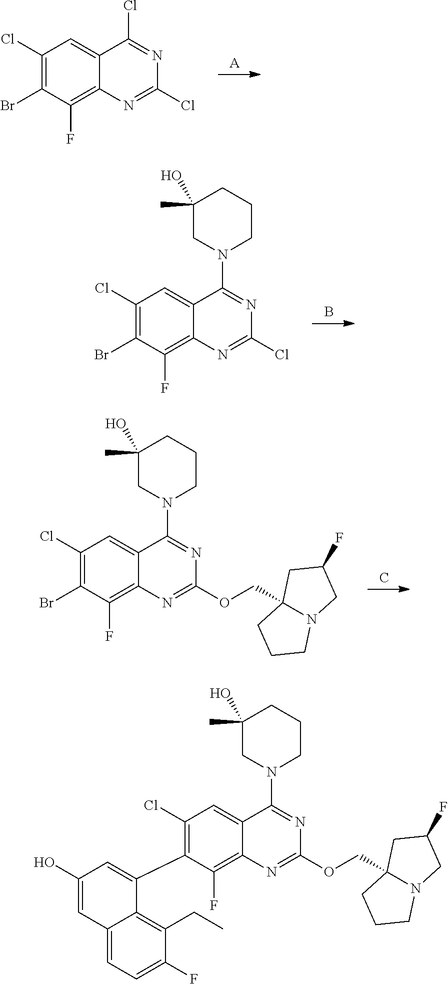

Step A. (R)-1-(7-bromo-2,6-dichloro-8-fluoroquinazolin-4-yl)-3-methylpiperidin-3-ol: To a solution of 7-bromo-2,4,6-trichloro-8-fluoroquinazoline (723 mg, 1.0 equiv) and DIEA (1.41 g, 5.0 equiv), 4 Å MS (100 mg) in DCM (10 mL) was added (R)-3-methylpiperidin-3-ol (302 mg, 1.2 equiv) in DCM (1 mL) dropwise at 0° C. The reaction was stirred at 0-15° C. for 0.5 hour. After completion, the reaction mixture was diluted with H₂O (10 mL) and extracted with DCM (20 mL). The combined organic layer was dried over Na₂SO₄, filtered and concentrated under reduced pressure. The residue was purified by column chromatography (SiO₂, Petroleum ether/Ethyl acetate=5/1 to 1/1) to give the title compound (541 mg, 59% yield, 98.7% purity) as yellow solid; LCMS (ESI, M+1): m/z=409.9.

Step B. (R)-1-(7-bromo-6-chloro-8-fluoro-2-(((2R,7aS)-2-fluorohexahydro-1H-pyrrolizin-7a-yl)methoxy)quinazolin-4-yl)-3-methylpiperidin-3-ol: To a mixture of (R)-1-(7-bromo-2,6-dichloro-8-fluoroquinazolin-4-yl)-3-methylpiperidin-3-ol (478 mg, 1.0 equiv), ((2R,7aS)-2-fluorohexahydro-1H-pyrrolizin-7a-yl)methanol (558 mg, 3.0 equiv), 4 Å molecular sieve (50 mg) in dioxane (10 mL) was added DIEA (755 mg, 5.0 equiv). The reaction was stirred at 90° C. for 16 hours. After completion, the reaction mixture was diluted with H₂O (10 mL) and extracted with Ethyl acetate (10 mL). The combined organic layer was dried over Na₂SO₄, filtered and concentrated under reduced pressure to give a residue. The crude product was purified by reversed-phase flash (0.1% FA condition) to afford the title compound (214 mg, 32% yield, 93.0% purity) as light yellow solid; LCMS (ESI, M+1): m/z=533.1.

Step C. (3R)-1-(6-chloro-7-(8-ethyl-7-fluoro-3-hydroxynaphthalen-1-yl)-8-fluoro-2-(((2R,7aS)-2-fluorohexahydro-1H-pyrrolizin-7a-yl)methoxy)guinazolin-4-yl)-3-methylpiperidin-3-ol: To a mixture of (R)-1-(7-bromo-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (100 mg, 1.0 equiv), 5-ethyl-6-fluoro-4-(4,4,5,5-tetramethyl-1,3,2-dioxaborolan-2-yl)naphthalen-2-ol (71.3 mg, 1.2 equiv), K₂CO₃ (77.9 mg, 3.0 equiv) in dioxane (1.5 mL) and H₂O (0.5 mL) was added RuPhos Pd G3 (15.7 mg, 0.1 equiv) and RuPhos (8.77 mg, 0.1 equiv) under N₂. The reaction was stirred at 80° C. for 1.5 hours. After completion, the reaction mixture was diluted with H₂O (2 mL) and extracted with Ethyl acetate (4 mL). The combined organic layer was dried over Na₂SO₄, filtered and concentrated under reduced pressure to give a residue. The crude product was purified by reversed-phase flash (0.1% FA condition) and prep-HPLC (Phenomenex Synergi C18 150×25 mm×10 μm; A: water (0.225% FA), B: ACN, B %: 19%-49% over 10 min) to afford the title compound (4.42 mg, 3% yield) as white solid; ¹H NMR (400 MHz, methanol-d₄): δ=8.57-8.48 (m, 1H), 8.17-8.09 (m, 1H), 7.67 (dd, J=6.0, 9.2 Hz, 1H), 7.28 (d, J=2.8 Hz, 1H), 7.24 (t, J=9.6 Hz, 1H), 6.89 (d, J=2.8 Hz, 1H), 5.47-5.29 (m, 1H), 4.60 (br s, 1H), 4.45-4.38 (m, 1H), 4.36-4.31 (m, 1H), 4.10 (br d, J=14.0 Hz, 1H), 3.57 (d, J=13.2 Hz, 1H), 3.52-3.34 (m, 4H), 3.18-3.10 (m, 1H), 2.68-2.54 (m, 1H), 2.52-2.04 (m, 7H), 2.01-1.92 (m, 1H), 1.88-1.71 (m, 3H), 1.32-1.25 (m, 3H), 0.84-0.74 (m, 3H); LCMS (ESI, M+1): m/z=641.2.

### Example 25

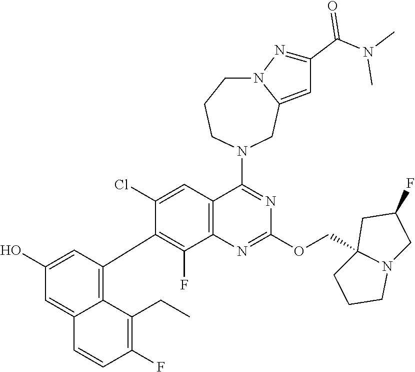 [SMILES: CCc1c(F)ccc2cc(O)cc(-c3c(Cl)cc4c(N5CCCn6nc(C(=O)N(C)C)cc6C5)nc(OC[C@@]56CCCN5C[C@H](F)C6)nc4c3F)c12]

### 5-(6-chloro-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

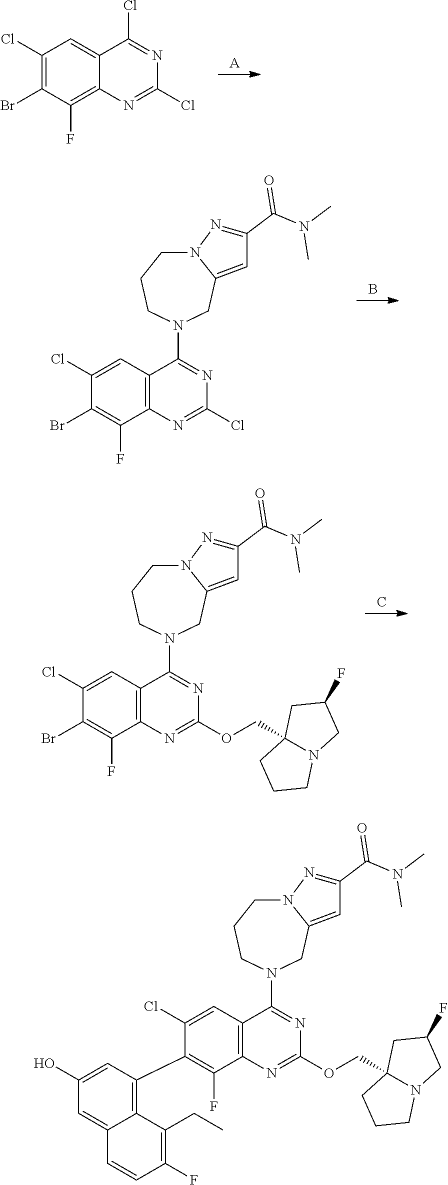

Step A. 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide: To a solution of 7-bromo-2,4,6-trichloro-8-fluoro-quinazoline (400 mg, 1.0 equiv) in DCM (4 mL) was added DIEA (782 mg, 5.0 equiv) at 0° C. The mixture was added N,N-dimethyl-5,6,7,8-tetrahydro-4H-pyrazolo[1,5-a][1,4]diazepine-2-carboxamide (385 mg, 1.3 equiv, HCl) and stirred at 25° C. for 0.5 hour. The reaction mixture was extracted with dichloromethane (2×10 mL), dried over anhydrous sodium sulfate, concentrated to afford the title compound (515 mg, crude) as white solid. LCMS (ESI, M+1): m/z=503.1.

Step B. 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide (515 mg, 1.0 equiv) in dioxane (5 mL) was added DIEA (398 mg, 3.0 equiv) and [(2R,8S)-2-fluoro-1,2,3,5,6,7-hexahydropyrrolizin-8-yl]methanol (816 mg, 5.0 equiv). The mixture was stirred at 90° C. for 36 hours. The reaction mixture was filtered and purified by reversed phase flash [C18, 0.1% formic acid condition] to afford the title compound (301 mg, 46% yield) as yellow solid; LCMS (ESI, M+1): m/z=625.8.

Step C. 5-(6-chloro-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)guinazolin-4-yl)-N,N-dimethyl-5,6,7,8-tetrahydro-4H-pyrazolo[1,5-a][1,4]diazepine-2-carboxamide: A mixture of 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide (300 mg, 1.0 equiv), 5-ethyl-6-fluoro-4-(4,4,5,5-tetramethyl-1,3,2-dioxaborolan-2-yl)naphthalen-2-ol (273 mg, 1.8 equiv), RuPhos Pd G3 (40.2 mg, 0.1 equiv), RuPhos (67.21 mg, 0.3 equiv), Cs₂CO₃ (469 mg, 3.0 equiv) in dioxane (3 mL) and H₂O (1 mL) was degassed and purged with N₂ for 3 times, and then the mixture was stirred at 80° C. for 6 hours under N₂ atmosphere. The reaction mixture was extracted with ethyl acetate (3×10 mL), dried over anhydrous sodium sulfate, concentrated. The residue was purified by prep-HPLC [Unisil 150×50 mm×3 um; A: water (FA), B: ACN; B %: 15%-45% over 10 min] to afford the title compound (100 mg, 27% yield, 0.7 FA) as white solid. ¹H NMR (400 MHz, METHANOL-d₄) δ=8.09 (s, 1H), 7.70-6.65 (m, 1H), 7.31-7.21 (m, 2H), 6.89 (d, J=2.4 Hz, 1H), 6.74 (s, 1H), 5.48-5.30 (m, 1H), 5.21-5.06 (m, 2H), 4.57-4.50 (m, 2H), 4.43-4.27 (m, 4H), 3.60-3.41 (m, 3H), 3.35 (s, 3H), 3.19-3.13 (m, 1H), 3.08 (s, 3H), 2.67-2.57 (m, 1H), 2.51-2.31 (m, 4H), 2.29-2.21 (m, 2H), 2.16-2.06 (m, 2H), 2.05-1.93 (m, 1H), 0.78 (dt, J=2.0, 7.4 Hz, 3H); LCMS (ESI, M+1): m/z=734.2.

### Example 26

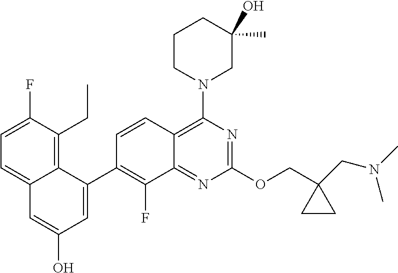 [SMILES: CCc1c(F)ccc2cc(O)cc(-c3ccc4c(N5CCC[C@@](C)(O)C5)nc(OCC5(CN(C)C)CC5)nc4c3F)c12]

### (R)-1-(2-((1-((dimethylamino)methyl)cyclopropyl)methoxy)-7-(8-ethyl-7-fluoro-3-hydroxynaphthalen-1-yl)-8-fluoroquinazolin-4-yl)-3-methylpiperidin-3-ol

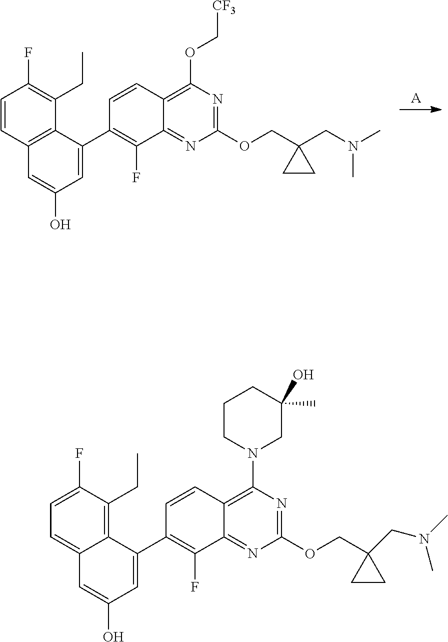

Step A. (R)-1-(2((1-((dimethylamino)methyl)cyclopropyl)methoxy)-7-(8-ethyl-7-fluoro-3-hydroxynaphthalen-1-yl)-8-fluoroquinazolin-4-yl)-3-methylpiperidin-3-ol: To a mixture of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (100 mg, 1.0 equiv), (3R)-3-methylpiperidin-3-ol (41.0 mg, 2.0 equiv) and 4 Å molecular sieve (10 mg) in DMF (1 mL) was added DIEA (69.0 mg, 3.0 equiv). The mixture was stirred at 60° C. for 24 hours. The mixture was filtered and washed with DMF (1 mL). The residue was purified by prep-HPLC [Phenomenex C18 75×30 mm×3 μm; A: water (FA), B: ACN, B %: 18%-48% over 7 min] to afford the title compound (58.4 mg, 56% yield) as yellow solid; SFC: Rt=0.992 min, 1.715 min; (column: Chiralpak IC-3 50×4.6 mm I.D., 3 um; mobile phase: phase A for CO₂, and phase B for MeOH (0.05% DEA); gradient elution: 40% MeOH (0.05% DEA) in CO₂; flow rate: 3 mL/min; detector: PDA; column temp: 35° C.; back pressure: 100 Bar). ¹H NMR (400 MHz, methanol-d₄) δ=7.97 (d, J=8.6 Hz, 1H), 7.65 (dd, J=6.0, 9.2 Hz, 1H), 7.31 (dd, J=7.2, 8.4 Hz, 1H), 7.27-7.18 (m, 2H), 6.95 (d, J=1.6 Hz, 1H), 4.48-4.35 (m, 2H), 4.28 (br d, J=12.0 Hz, 1H), 4.12 (br d, J=13.2 Hz, 1H), 3.63-3.40 (m, 2H), 3.15-2.98 (m, 2H), 2.78 (s, 6H), 2.45 (dt, J=3.2, 7.2 Hz, 2H), 2.23-2.06 (m, 1H), 1.89-1.71 (m, 3H), 1.27 (d, J=12.8 Hz, 3H), 0.94-0.85 (m, 2H), 0.82-0.72 (m, 5H); LCMS (ESI, M+1): m/z=577.3.

### Example 27

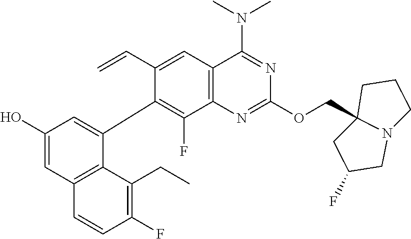 [SMILES: C=Cc1cc2c(N(C)C)nc(OC[C@@]34CCCN3C[C@H](F)C4)nc2c(F)c1-c1cc(O)cc2ccc(F)c(CC)c12]

### 4-(4-(dimethylamino)-8-fluoro-2-(((2R,7aS)-2-fluorotetrahydro-1H-pyrrolizin-7a(5H)-yl)methoxy)-6-vinylquinazolin-7-yl)-5-ethyl-6-fluoronaphthalen-2-ol

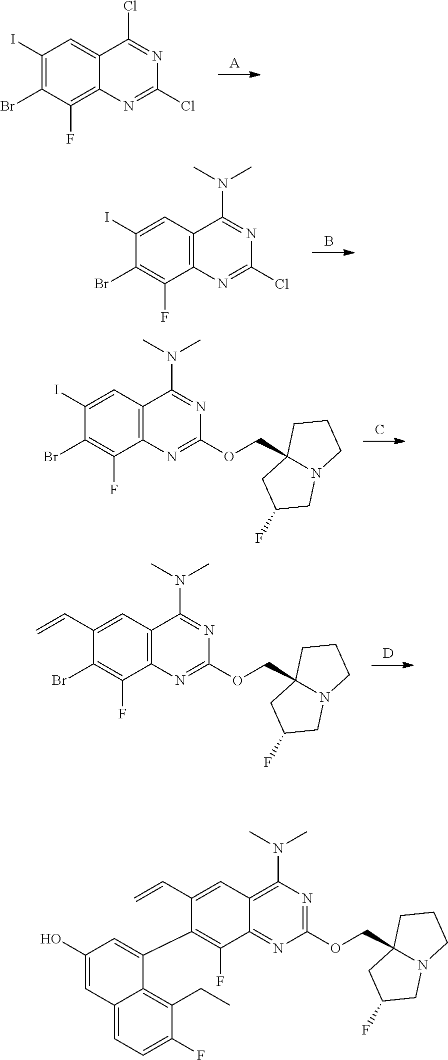

Step A. 7-bromo-2-chloro-8-fluoro-6-iodo-N,N-dimethylquinazolin-4-amine: To a solution of 7-bromo-2,4-dichloro-8-fluoro-6-iodo-quinazoline (9.20 g, 1.0 equiv) and DIEA (8.46 g, 3.0 equiv) in THE (80 mL) was added dropwise N-methylmethanamine (2 M, 21.8 mL, 2.0 equiv). The mixture was stirred at −40° C. for 0.5 hour. The reaction mixture was dissolved in DCM (700 mL). The mixture was diluted with water (10 mL) and extracted with DCM (30 mL×3). The organic layers were dried with anhydrous sodium sulfate, concentrated and triturated with MTBE (100 mL) at 20° C. for 20 mins to afford the title compound (8.00 g, 85% yield) as yellow solid.

Step B. 7-bromo-8-fluoro-2-(((2R,7aS)-2-fluorohexahydro-1H-pyrrolizin-7a-yl)methoxy)-6-iodo-N,N-dimethylquinazolin-4-amine: To a solution of 7-bromo-2-chloro-8-fluoro-6-iodo-N,N-dimethyl-quinazolin-4-amine (8.00 g, 1.0 equiv), ((2R,7aS)-2-fluorotetrahydro-1H-pyrrolizin-7a(5H)-yl)methanol (8.88 g, 3.0 equiv) in dioxane (40 mL) was added DIEA (7.21 g, 3.0 equiv). The mixture was stirred at 100° C. for 96 hours. The reaction mixture was poured into water (200 mL) to form a solid. The solid was filtered and the filtered cake was triturated with PE (200 mL) at 20° C. for 1 hour to afford the title compound (7.70 g, 66% yield) as yellow solid; LCMS (ESI, M+3): m/z=554.9.

Step C. 7-bromo-8-fluoro-2-(((2R,7aS)-2-fluorotetrahydro-1H-pyrrolizin-7a(5H)-yl)methoxy)-N,N-dimethyl-6-vinylquinazolin-4-amine: To a solution of 7-bromo-8-fluoro-2-(((2R,7aS)-2-fluorohexahydro-1H-pyrrolizin-7a-yl)methoxy)-6-iodo-N,N-dimethylquinazolin-4-amine (1.00 g, 1.0 equiv) in dioxane (10 mL) and H₂O (1 mL) was added 4,4,5,5-tetramethyl-2-vinyl-1,3,2-dioxaborolane (334 mg, 1.2 equiv), K₂CO₃ (749 mg, 3.0 equiv), Pd(dppf)Cl₂ (13.2 mg, 0.01 equiv) and degassed and purged with N₂ for 3 times, and then the mixture was stirred at 40° C. for 16 hours under N₂ atmosphere. The mixture was poured into water (20 mL) and filtered. The filtrate was extracted with ethyl acetate (2×20 mL). The organic phase was dried over Na₂SO₄ and concentrated under vacuum. The crude product was purified by reversed phase flash [C18, 0.1% formic acid] to afford the title compound (300 mg, 35% yield) as yellow solid; LCMS (ESI, M+3): m/z=455.0.

Step D. 4-(4-(dimethylamino)-8-fluoro-2-(((2R, 7aS)-2-fluorotetrahydro-1H-pyrrolizin-7a(5H)-yl)methoxy)-6-vinylquinazolin-7-yl)-5-ethyl-6-fluoronaphthalen-2-ol: To a solution of 7-bromo-8-fluoro-2-(((2R,7aS)-2-fluorotetrahydro-1H-pyrrolizin-7a(5H)-yl)methoxy)-N,N-dimethyl-6-vinylquinazolin-4-amine (1.80 g, 1.0 equiv) and 5-ethyl-6-fluoro-4-(4,4,5,5-tetramethyl-1,3,2-dioxaborolan-2-yl)naphthalen-2-ol (1.37 g, 1.2 equiv) in methoxycyclopentane (18 mL) was added Ad2nBuP-Pd-G3 (393 mg, 0.15 equiv) and Cs₂CO₃ (326 mg, 3 equiv). The mixture was stirred at 90° C. for 12 hours. The mixture was poured into water (20 mL) and filtered. The filtrate was extracted with ethyl acetate (2×20 mL). The organic phase was dried over Na₂SO₄ and concentrated under vacuum. The crude product purified by prep-HPLC [Phenomenex luna C18 150×40 mm×5 μm; A: water (FA), B:ACN; B %: 20%-50% over 10 min) to afford the title compound (475 mg, 21% yield) as yellow solid; ¹H NMR (400 MHz, METHANOL-d₄) δ=8.58-8.46 (m, 1H), 8.26 (s, 1H), 7.77-7.60 (m, 1H), 7.32-7.16 (m, 2H), 6.93-6.80 (m, 1H), 6.37-6.17 (m, 1H), 5.78-5.60 (m, 1H), 5.53-5.27 (m, 1H), 5.19-5.05 (m, 1H), 4.86 (s, 9H), 4.53-4.34 (m, 2H), 3.73-3.56 (m, 1H), 3.55 (s, 6H), 3.44 (s, 2H), 3.24-3.16 (m, 1H), 2.59-2.34 (m, 3H), 2.31-2.20 (m, 2H), 2.19-2.09 (m, 2H), 2.05-1.95 (m, 1H), 0.81-0.67 (m, 3H); LCMS (ESI, M+1): m/z=563.2.

### Example 28

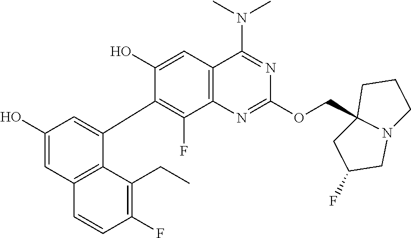 [SMILES: CCc1c(F)ccc2cc(O)cc(-c3c(O)cc4c(N(C)C)nc(OC[C@@]56CCCN5C[C@H](F)C6)nc4c3F)c12]

### 4-(dimethylamino)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-6-ol

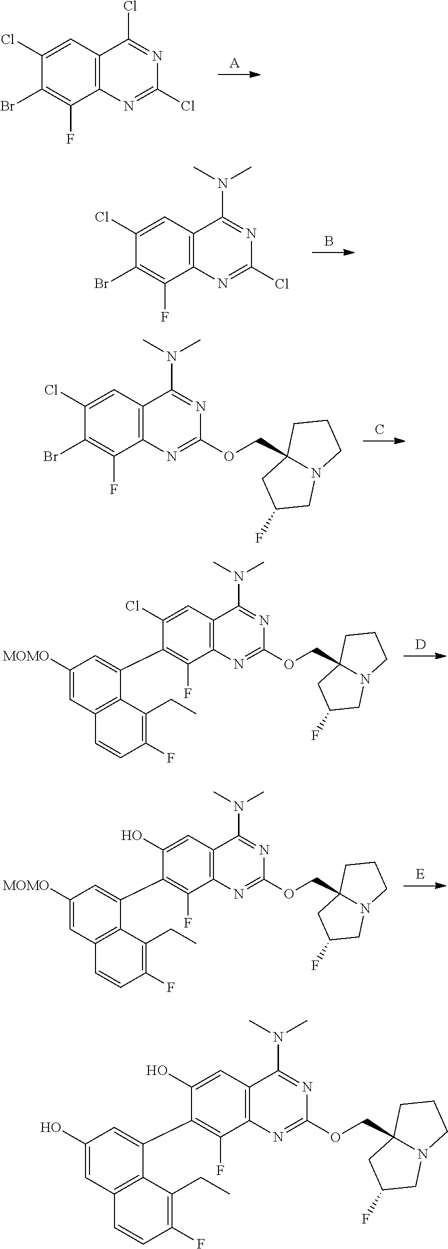

Step A. 7-bromo-2,6-dichloro-8-fluoro-N,N-dimethyl-quinazolin-4-amine: To a solution of 7-bromo-2,4,6-trichloro-8-fluoro-quinazoline (5.1 g, 1.0 equiv) in THE (50 mL) was added DIEA (5.99 g, 8.07 mL, 3.0 equiv) and N-methylmethanamine (2 M, 23.16 mL, 3.0 equiv). The mixture was stirred at −40° C. for 0.5 hours. The mixture was poured into water (20 mL) and filtered. The filtrate was extracted with ethyl acetate (2×50 mL). The organic phase was dried over Na₂SO₄ and concentrated under vacuum. The crude product was purified by slurried by acetonitrile (20 mL) at 25° C. for 0.5 hour afford the title compound (6.2 g, 88% yield) as white solid; LCMS (ESI, M+1): m/z=339.8.

Step B. 7-bromo-6-chloro-8-fluoro-2-(((2R,7aS)-2-fluorohexahydro-1H-pyrrolizin-7a-yl)methoxy)-N,N-dimethylquinazolin-4-amine: To a mixture of 7-bromo-2,6-dichloro-8-fluoro-N,N-dimethyl-quinazolin-4-amine (6.2 g, 1.0 equiv) was added ((2R,7aS)-2-fluorotetrahydro-1H-pyrrolizin-7a(5H)-yl)methanol (14.6 g, 5.0 equiv). The mixture was stirred at 90° C. for 24 hours. The mixture was filtered. The crude product was purified by re-crystallization from acetonitrile afford the title compound (4.1 g, 48% yield) as white solid; LCMS (ESI, M+1): m/z=462.9.

Step C. 6-chloro-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-N,N-dimethylquinazolin-4-amine: A mixture of 7-bromo-6-chloro-8-fluoro-2-(((2R,7aS)-2-fluorohexahydro-1H-pyrrolizin-7a-yl)methoxy)-N,N-dimethylquinazolin-4-amine (4.3 g, 1.0 equiv), 2-[8-ethyl-7-fluoro-3-(methoxymethoxy)-1-naphthyl]-4,4,5,5-tetramethyl-1,3,2-dioxaborolane (5.03 g, 1.5 equiv), RuPhos-Pd-G3 (778.89 mg, 0.1 equiv), RuPhos (1.30 g, 0.3 equiv) and Cs₂CO₃ (9.10 g, 3.0 equiv) in dioxane (32 mL) and H₂O (8 mL) was degassed and purged with N₂ for 3 times, and then the mixture was stirred at 90° C. for 3 hours under N₂ atmosphere. The mixture was poured into water (10 mL) and filtered. The filtrate was extracted with ethyl acetate (4×30 mL). The organic phase was dried over Na₂SO₄ and concentrated under vacuum. The crude product was purified with reversed phase flash [C18, 0.1% formic acid] afford the title compound (1.6 g, 28% yield) as yellow solid; LCMS (ESI, M+1): m/z=615.3.

Step D. 4-(dimethylamino)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)guinazolin-6-ol: A mixture of 6-chloro-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)-N,N-dimethylquinazolin-4-amine (640 mg, 1.0 equiv), Pd₂(dba)₃ (95.3 mg, 0.1 equiv), t-Bu Xphos (88.4 mg, 0.2 equiv) and KOH (175 mg, 3.0 equiv) in dioxane (5 mL) and H₂O (1 mL) was degassed and purged with N₂ for 3 times, and then the mixture was stirred at 90° C. for 12 hours under N₂ atmosphere. The mixture was poured into water (2 mL) and filtered. The filtrate was extracted with ethyl acetate (4×5 mL). The organic phase was dried over Na₂SO₄ and concentrated under vacuum. The crude product was purified with reversed phase flash [C18, 0.1% formic acid] afford the title compound (120 mg, 19% yield) as yellow solid; LCMS (ESI, M+1): m/z=597.3.

Step E. 4-(dimethylamino)-7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)guinazolin-6-ol: To a solution of 4-(dimethylamino)-7-(8-ethyl-7-fluoro-3-(methoxymethoxy)naphthalen-1-yl)-8-fluoro-2-(((2R,7aS)-2-fluorotetrahydro-1H-pyrrolizin-7a(5H)-yl)methoxy)quinazolin-6-ol (35 mg, 1.0 equiv) in ACN (1 mL) was added HCl.dioxane (4 M, 1 mL) at 0° C. The mixture was stirred at 25° C. for 1 hour. The reaction mixture was concentrated under vacuum. The crude product was purified with prep-HPLC [Phenomenex luna C18 150×25 mm×10 um; A: water(FA); B: ACN; B %: 14%-34% over 2 min] afford the title compound (120 mg, 19% yield) as light yellow solid; ¹H NMR (400 MHz, METHANOL-d₄) δ=7.65-7.61 (m, 1H), 7.47 (d, J=1.2 Hz, 1H), 7.28-7.16 (m, 2H), 6.92 (d, J=2.4 Hz, 1H), 5.34 (s, 1H), 4.52-4.37 (m, 2H), 3.73-3.53 (m, 3H), 3.53-3.41 (m, 6H), 3.24 (d, J=5.6 Hz, 1H), 2.60-2.37 (m, 4H), 2.35-2.25 (m, 1H), 2.21-2.10 (m, 2H), 2.01 (d, J=4.4 Hz, 1H), 0.85-0.77 (m, 3H); LCMS (ESI, M+1): m/z=553.4.

### Example 29

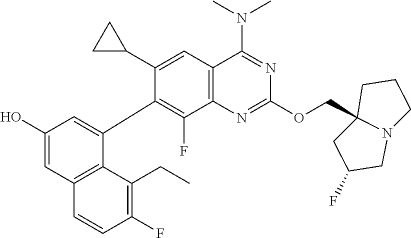 [SMILES: CCc1c(F)ccc2cc(O)cc(-c3c(C4CC4)cc4c(N(C)C)nc(OC[C@@]56CCCN5C[C@H](F)C6)nc4c3F)c12]

### 4-(6-cyclopropyl-4-(dimethylamino)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol

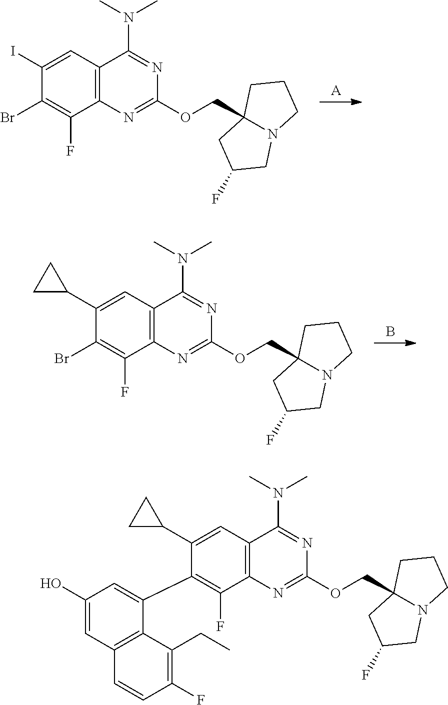

Step A. 7-bromo-6-cyclopropyl-8-fluoro-2-(((2R,7aS)-2-fluorotetrahydro-1H-pyrrolizin-7a(5H)-yl)methoxy)-N,N-dimethylquinazolin-4-amine: A mixture of 7-bromo-8-fluoro-2-(((2R,7aS)-2-fluorohexahydro-1H-pyrrolizin-7a-yl)methoxy)-6-iodo-N,N-dimethylquinazolin-4-amine (500 mg, 1.0 equiv), cyclopropylboronic acid (233 mg, 3.0 equiv), Pd(dppf)Cl₂ (66.1 mg, 0.1 equiv), K₃PO₄ (1.5 M, 1.8 mL, 3.0 equiv) in dioxane (5 mL) was degassed and purged with N₂ for 3 times, and then the mixture was stirred at 80° C. for 12 hours under N₂ atmosphere. The mixture was poured into water (2 mL) and filtered. The filtrate was extracted with ethyl acetate (3×40 mL). The organic phase was dried over Na₂SO₄ and concentrated under vacuum. The crude product was purified with prep-HPLC [YMC Triart C18 150×25 mm×5 um; A: water (FA); B: ACN; B %: 18%-48% over 10 min] to afford the title compound (92 mg, 22% yield) as white solid. LCMS (ESI, M+1): m/z=467.1.

Step B. 4-(6-cyclopropyl-4-(dimethylamino)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol: A mixture of 7-bromo-6-cyclopropyl-8-fluoro-2-(((2R,7aS)-2-fluorotetrahydro-1H-pyrrolizin-7a(5H)-yl)methoxy)-N,N-dimethylquinazolin-4-amine (82 mg, 1.0 equiv), 5-ethyl-6-fluoro-4-(4,4,5,5-tetramethyl-1,3,2-dioxaborolan-2-yl)naphthalen-2-ol (83.2 mg, 1.50 equiv), RuPhos-Pd-G3 (14.7 mg, 0.1 equiv), RuPhos (24.6 mg, 0.3 equiv) and Cs₂CO₃ (171 mg, 3.0 equiv) in dioxane (5 mL) and H₂O (1.0 mL) was degassed and purged with N₂ for 3 times, and then the mixture was stirred at 90° C. for 12 hours under N₂ atmosphere. The mixture was poured into water (1.0 mL) and filtered. The filtrate was extracted with ethyl acetate (3×2 mL). The organic phase was dried over Na₂SO₄ and concentrated under vacuum. The crude product was purified with prep-HPLC [Phenomenex luna C18 150×25 mm×10 um; A; water(FA); B: ACN; B %: 22%-52% over 10 min] afford the title compound (14.6 mg, 14% yield) as white solid; ¹H NMR (400 MHz, METHANOL-d₄) δ=7.66-7.62 (m, 1H), 7.49 (s, 1H), 7.32-7.17 (m, 2H), 6.94 (d, J=2.6 Hz, 1H), 5.46-5.25 (m, 1H), 4.43-4.25 (m, 2H), 3.62-3.49 (m, 1H), 3.46 (s, 6H), 3.40-3.33 (m, 2H), 3.15-3.06 (m, 1H), 2.65-2.53 (m, 1H), 2.48-2.17 (m, 4H), 2.11-2.01 (m, 2H), 1.99-1.86 (m, 1H), 1.51-1.39 (m, 1H), 0.80-0.56 (m, 7H); LCMS (ESI, M+1): m/z=577.5.

### Example 30

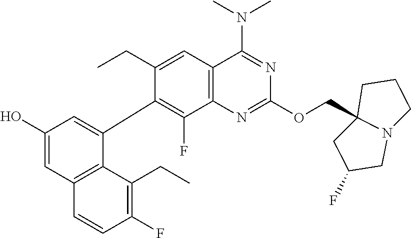 [SMILES: CCc1cc2c(N(C)C)nc(OC[C@@]34CCCN3C[C@H](F)C4)nc2c(F)c1-c1cc(O)cc2ccc(F)c(CC)c12]

### 4-(4-(dimethylamino)-6-ethyl-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol

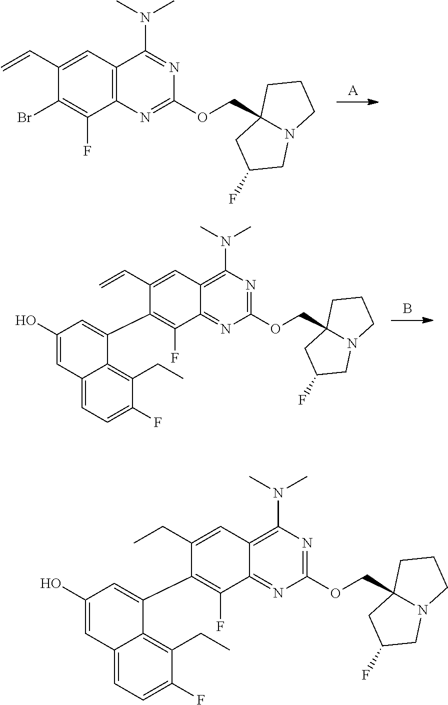

Step A. 4-(4-(dimethylamino)-8-fluoro-2-(((2R, 7aS)-2-fluorotetrahydro-1H-pyrrolizin-7a(5H)-yl)methoxy)-6-vinylquinazolin-7-yl)-5-ethyl-6-fluoronaphthalen-2-ol: To a solution of 7-bromo-8-fluoro-2-(((2R,7aS)-2-fluorotetrahydro-1H-pyrrolizin-7a(5H)-yl)methoxy)-N,N-dimethyl-6-vinylquinazolin-4-amine (1.80 g, 1.0 equiv) and 5-ethyl-6-fluoro-4-(4,4,5,5-tetramethyl-1,3,2-dioxaborolan-2-yl)naphthalen-2-ol (1.37 g, 1.2 equiv) in methoxycyclopentane (18 mL) was added Ad2nBuP-Pd-G3 (393 mg, 0.15 equiv) and Cs₂CO₃ (326 mg, 3.0 equiv). The mixture was stirred at 90° C. for 12 hours. The mixture was poured into water (20 mL) and filtered. The filtrate was extracted with ethyl acetate (2×20 mL). The organic phase was dried over Na₂SO₄ and concentrated under vacuum. The crude product purified by column chromatography [SiO₂, Petroleum ether/Ethyl acetate=10/1 to 1/1] to afford the title compound (475 mg, 21% yield) as yellow solid; ¹H NMR (400 MHz, METHANOL-d₄) δ=8.58-8.46 (m, 1H), 8.26 (s, 1H), 7.77-7.60 (m, 1H), 7.32-7.16 (m, 2H), 6.93-6.80 (m, 1H), 6.37-6.17 (m, 1H), 5.78-5.60 (m, 1H), 5.53-5.27 (m, 1H), 5.19-5.05 (m, 1H), 4.86 (s, 9H), 4.53-4.34 (m, 2H), 3.73-3.56 (m, 1H), 3.55 (s, 6H), 3.44 (br s, 2H), 3.24-3.16 (m, 1H), 2.59-2.34 (m, 3H), 2.31-2.20 (m, 2H), 2.19-2.09 (m, 2H), 2.05-1.95 (m, 1H), 0.81-0.67 (m, 3H); LCMS (ESI, M+1): m/z=563.2.

Step B. 4-(4-(dimethylamino)-6-ethyl-8-fluoro-2-(((2R, 7aS)-2-fluorotetrahydro-1H-pyrrolizin-7a(5H)-yl)methoxy)quinazolin-7-yl)-5-ethyl-6-fluoronaphthalen-2-ol: To a Pd/C (20.0 mg, 10% purity) in MeOH (4 mL) was added 4-(4-(dimethylamino)-8-fluoro-2-(((2R, 7aS)-2-fluorotetrahydro-1H-pyrrolizin-7a(5H)-yl)methoxy)-6-vinylquinazolin-7-yl)-5-ethyl-6-fluoronaphthalen-2-ol (200 mg, 1.0 equiv) at N₂. The mixture was degassed and purged with H₂ for 3 times, and then the mixture was stirred at 25° C. for 12 hours under H₂ atmosphere. The reaction mixture was filtered and concentrated under reduced pressure to give a residue. The residue was purified with prep-HPLC [Phenomenex Synergi C18 150×25 mm×10 μm; A: water (FA), B:ACN; B %: 19%-49% over 10 min) to afford the title compound (31 mg, 15% yield) as light yellow solid; ¹H NMR (400 MHz, METHANOL-d₄) δ=8.58-8.48 (m, 1H), 7.97-7.88 (m, 1H), 7.72-7.62 (m, 1H), 7.33-7.17 (m, 2H), 6.94-6.86 (m, 1H), 5.51-5.31 (m, 1H), 4.55-4.36 (m, 2H), 3.71-3.54 (m, 2H), 3.52 (s, 6H), 3.50-3.44 (m, 1H), 3.26-3.17 (m, 1H), 2.62-2.45 (m, 2H), 2.45-2.25 (m, 4H), 2.22-2.10 (m, 3H), 2.06-1.94 (m, 1H), 1.11-0.98 (m, 3H), 0.79-0.70 (m, 3H); LCMS (ESI, M+1): m/z=565.2.

### Example 31

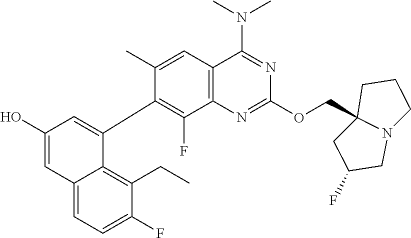 [SMILES: CCc1c(F)ccc2cc(O)cc(-c3c(C)cc4c(N(C)C)nc(OC[C@@]56CCCN5C[C@H](F)C6)nc4c3F)c12]

### 4-(4-(dimethylamino)-8-fluoro-2-(((2R,7aS)-2-fluorotetrahydro-1H-pyrrolizin-7a(5H)-yl)methoxy)-6-methylquinazolin-7-yl)-5-ethyl-6-fluoronaphthalen-2-ol

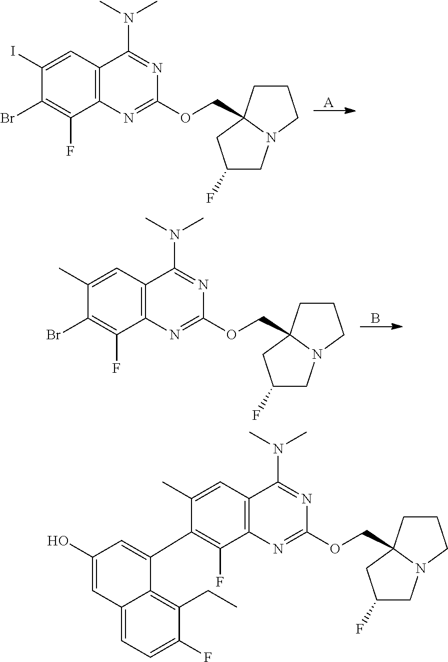

Step A. 7-bromo-8-fluoro-2-(((2R,7aS)-2-fluorohexahydro-1H-pyrrolizin-7a-yl)methoxy)-N,N,6-trimethylquinazolin-4-amine: A mixture of 7-bromo-8-fluoro-2-(((2R,7aS)-2-fluorohexahydro-1H-pyrrolizin-7a-yl)methoxy)-6-iodo-N,N-dimethylquinazolin-4-amine (500 mg, 1 equiv), 2,4,6-trimethyl-1,3,5,2,4,6-trioxatriborinane (516 μL, 2 equiv) and K₃PO₄ (1.5 M, 1.8 mL, 3 equiv), Pd(dppf)Cl₂ (66 mg, 0.1 equiv) in dioxane (5 mL) was degassed and purged with N₂ for 3 times. The mixture was stirred at 50° C. for 12 hours under N₂ atmosphere. The mixture was diluted with water (10 mL) and extracted with ethyl acetate (4×10 mL). The organic phase was dried over Na₂SO₄ and concentrated under vacuum. The crude product was purified with reversed phase flash [C18, 0.1% formic acid condition] and prep-TLC (Dichloromethane/Methanol=5/1) to afford the title compound (50 mg, 12% yield) as white solid; LCMS (ESI, M+1): m/z=441.1.

Step B. 4-(4-(dimethylamino)-8-fluoro-2-(((2R,7aS)-2-fluorotetrahydro-1H-pyrrolizin-7a(5H)-yl)methoxy)-6-methylquinazolin-7-yl)-5-ethyl-6-fluoronaphthalen-2-ol: A mixture of 7-bromo-8-fluoro-2-(((2R,7aS)-2-fluorotetrahydro-1H-pyrrolizin-7a(5H)-yl)methoxy)-N,N,6-trimethylquinazolin-4-amine (50 mg, 1 equiv), 5-ethyl-6-fluoro-4-(4,4,5,5-tetramethyl-1,3,2-dioxaborolan-2-yl)naphthalen-2-ol (53.7 mg, 1.5 equiv) in dioxane (1 mL), Cs₂CO₃ (110 mg, 3 equiv) in H₂O (0.2 mL), RuPhos (15.8 mg, 0.3 equiv) and RuPhos-Pd-G3 (9.5 mg, 0.1 equiv) was degassed and purged with N₂ for 3 times and then the mixture was stirred at 90° C. for 12 hours under N₂ atmosphere. The

mixture was diluted with water (5 mL), extracted with ethyl acetate (4×5 mL). The organic phase was dried over Na₂SO₄ and concentrated under vacuum. The crude product was purified with prep-HPLC [column: Phenomenex luna C18 150×25 mm×10 μm; A: water (FA), B: ACN; B %: 14%-44%, 2 min] and lyophilized to afford the title compound (19.2 mg, 30% yield) as white solid; ¹H NMR (400 MHz, METHANOL-d₄) δ=7.89 (s, 1H), 7.68-7.64 (m, 1H), 7.31-7.14 (m, 2H), 6.87 (d, J=2.4 Hz, 1H), 5.47-5.20 (m, 1H), 4.40-4.34 (m, 1H), 4.32-4.25 (m, 1H), 3.48-3.45 (s, 6H), 3.44-3.34 (m 3H), 3.14-3.04 (m, 1H), 2.66-2.53 (m, 1H), 2.48-2.25 (m, 2H), 2.24-2.15 (m, 2H), 2.11-1.99 (m, 5H), 1.99-1.88 (m, 1H), 0.77-0.73 (m, 3H); LCMS (ESI, M+1): m/z=551.3.

### Example 32

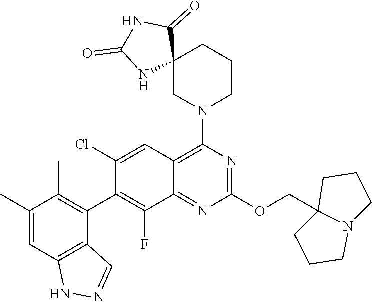 [SMILES: Cc1cc2[nH]ncc2c(-c2c(Cl)cc3c(N4CCC[C@]5(C4)NC(=O)NC5=O)nc(OCC45CCCN4CCC5)nc3c2F)c1C]

### (5R)-7-(6-chloro-7-(5,6-dimethyl-1H-indazol-4-yl)-8-fluoro-2-((tetrahydro-1H-pyrrolizin-7a(5H)-yl)methoxy)quinazolin-4-yl)-1,3,7-triazaspiro[4.5]decane-2,4-dione

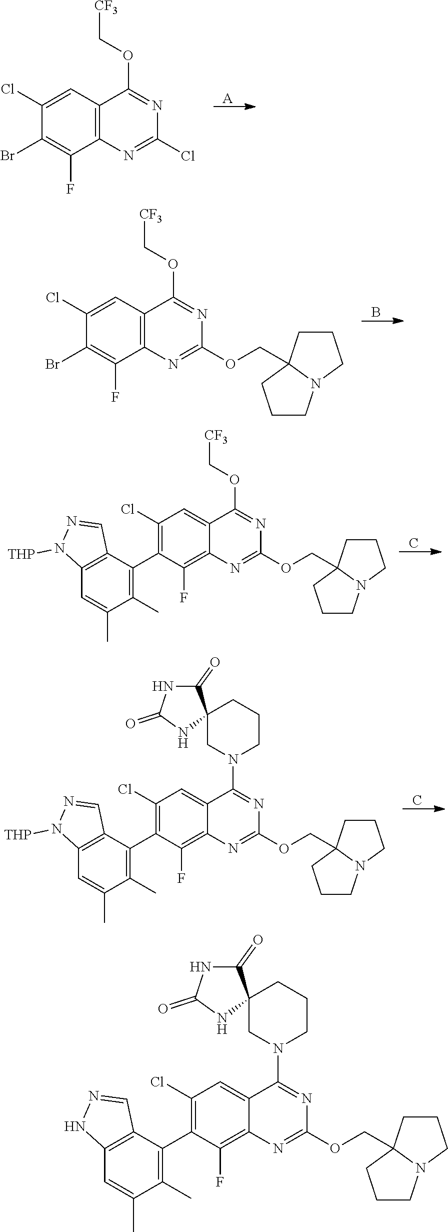

Step A. 7-bromo-6-chloro-8-fluoro-2-((hexahydro-1H-pyrrolizin-7a-yl)methoxy)-4-(2,2,2-trifluoroethoxy)quinazoline: To a mixture of 7-bromo-2,6-dichloro-8-fluoro-4-(2,2,2-trifluoroethoxy)quinazoline (500 mg, 1.0 equiv) in dioxane (5 mL) was added 1,2,3,5,6,7-hexahydropyrrolizin-8-ylmethanol (161 mg, 0.9 equiv), DIEA (656 mg, 4.0 equiv) and 4 Å molecular sieve (50 mg). The mixture was stirred at 40° C. for 16 hours. The reaction mixture was diluted with EtOAc (20 mL) and water (30 mL). The mixture was extracted with EtOAc (20 mL). The combined organic phase was washed with brine (20 mL), dried over anhydrous sodium sulfate, filtered and concentrated to give a residue. The residue was purified by reversed phase flash [water (0.1% FA)/acetonitrile]. The desired fractions were collected and neutralized with solid NaHCO₃, and concentrated under vacuum to remove acetonitrile. The aqueous layer was extracted with ethyl acetate (2×30 mL). The combined organic layers were dried over anhydrous sodium sulfate, and concentrated under vacuum to afford the title compound (210 mg, 33% yield) as yellow solid; ¹H NMR (400 MHz, DMSO-d₆) δ=8.02 (d, J=1.8 Hz, 1H), 5.35-5.23 (m, 2H), 4.14 (s, 2H), 3.00-2.90 (m, 2H), 2.58 (br s, 2H), 1.92-1.73 (m, 6H), 1.65-1.54 (m, 2H); LCMS (ESI, M+1, M+3): m/z=498.0, 500.0.

Step B. 6-chloro-7-(5,6-dimethyl-1-(tetrahydro-2H-pyran-2-yl)-1H-indazol-4-yl)-8-fluoro-2-((hexahydro-1H-pyrrolizin-7a-yl)methoxy)-4-(2,2,2-trifluoroethoxy)quinazoline: A mixture of 7-bromo-6-chloro-8-fluoro-2-((hexahydro-1H-pyrrolizin-7a-yl)methoxy)-4-(2,2,2-trifluoroethoxy)quinazoline (240 mg, 1.0 equiv), 5,6-dimethyl-1-tetrahydropyran-2-yl-4-(4,4,5,5-tetramethyl-1,3,2-dioxaborolan-2-yl)indazole (223 mg, 1.3 equiv), RuPhos Pd G3 (40.6 mg, 0.1 equiv), RuPhos (22.5 mg, 0.1 equiv) and Cs₂CO₃ (470 mg, 3.0 equiv) in dioxane (5 mL) and H₂O (1 mL) was degassed and purged with N₂ for 3 times, and then the mixture was stirred at 80° C. for 2 hours. The reaction mixture was diluted with water (20 mL). Then the mixture was extracted with EA (20 mL×3). The combined organic phase was washed with brine (20 mL), dried over anhydrous sodium sulfate, filtered and concentrated to give a residue. The residue was purified by reversed phase flash [water (0.1% FA)/acetonitrile]. The desired fractions were collected and neutralized with solid NaHCO₃ and concentrated under vacuum to remove acetonitrile. The aqueous layer was extracted with ethyl acetate (2×50 mL). The combined organic layers were dried over anhydrous sodium sulfate, and concentrated under vacuum to afford the title compound (130 mg, 35% yield) as yellow solid; LCMS (ESI, M+1): m/z=648.3.

Step C. (5R)-7-(6-chloro-7-(5,6-dimethyl-1-(tetrahydro-2H-pyran-2-yl)-1H-indazol-4-yl)-8-fluoro-2-((hexahydro-1H-pyrrolizin-7a-yl)methoxy)quinazolin-4-yl)-1,3,7-triazaspiro[4.5]decane-2,4-dione: To a solution of 6-chloro-7-(5,6-dimethyl-1-(tetrahydro-2H-pyran-2-yl)-1H-indazol-4-yl)-8-fluoro-2-((hexahydro-1H-pyrrolizin-7a-yl)methoxy)-4-(2,2,2-trifluoroethoxy)quinazoline (130 mg, 1.0 equiv) in DMF (1 mL) and ACN (1 mL) was added (5R)-1,3,9-triazaspiro[4.5]decane-2,4-dione (102 mg, 3.0 equiv) and K₃PO₄ (128 mg, 3.0 equiv). The reaction mixture was stirred at 40° C. for 16 hours. The mixture was filtered and the filtrate was purified by reversed phase flash [water (0.1% FA)/acetonitrile]. The desired fractions were collected and neutralized with solid NaHCO₃ and concentrated under vacuum to remove acetonitrile. The aqueous layer was extracted with ethyl acetate (2×20 mL). The combined organic layers were dried over anhydrous sodium sulfate and concentrated under vacuum to afford the title compound (60 mg, 39% yield) as yellow solid; LCMS (ESI, M+1): m/z=717.4.

Step D. (5R)-7-(6-chloro-7-(5,6-dimethyl-1H-indazol-4-yl)-8-fluoro-2-((hexahydro-1H-pyrrolizin-7a-yl)methoxy)quinazolin-4-yl)-1,3,7-triazaspiro[4.5]decane-2,4-dione: To a solution of (5R)-7-(6-chloro-7-(5,6-dimethyl-1-(tetrahydro-2H-pyran-2-yl)-1H-indazol-4-yl)-8-fluoro-2-((hexahydro-1H-pyrrolizin-7a-yl)methoxy)quinazolin-4-yl)-1,3,7-triazaspiro[4.5]decane-2,4-dione (50.0 mg, 1.0 equiv) in DCM (1 mL) was added TFA (2 mL, 387 equiv). The reaction mixture was stirred at 0° C. for 1 hour. Then the mixture was stirred at 20° C. for 16 hours. The mixture was filtered and the filtrate was concentrated under vacuum to give a residue. The residue was purified by prep-HPLC [column: Unisil 3-100 C18 Ultra 150×50 mm×3 μm; A: water (FA), B: ACN, B %: 10%-40% over 7 min]. The desired fraction was collected and concentrated under vacuum to remove acetonitrile and lyophilized to afford the title compound (60 mg, 39% yield) as yellow solid; ¹H NMR (400 MHz, METHANOL-d₄) δ=8.13 (s, 1H), 7.62-7.49 (m, 1H), 7.49-7.34 (m, 1H), 4.77-4.60 (m, 2H), 4.49 (br d, J=13.2 Hz, 1H), 4.39 (br d, J=13.2 Hz, 1H), 3.86-3.51 (m, 5H), 3.29-3.25 (m, 1H), 2.51 (s, 3H), 2.33 (br dd, J=6.8, 11.8 Hz, 3H), 2.28-2.15 (m, 6H), 2.14-2.06 (m, 5H), 2.01 (br s, 1H); LCMS (ESI, M+1): m/z=633.4.

### Example 33

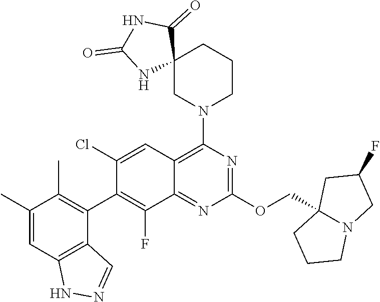 [SMILES: Cc1cc2[nH]ncc2c(-c2c(Cl)cc3c(N4CCC[C@]5(C4)NC(=O)NC5=O)nc(OC[C@@]45CCCN4C[C@H](F)C5)nc3c2F)c1C]

### (5R)-7-(6-chloro-7-(5,6-dimethyl-1H-indazol-4-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione

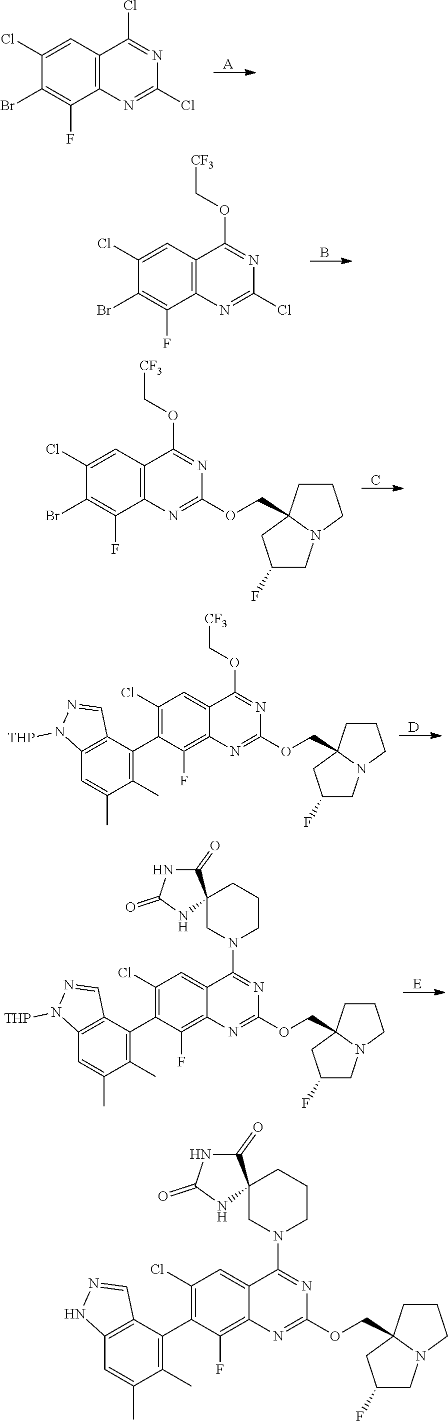

Step A. 7-bromo-2,6-dichloro-8-fluoro-4-(2,2,2-trifluoroethoxy)quinazoline: To a solution of 7-bromo-2,4,6-trichloro-8-fluoro-quinazoline (4.8 g, 1.0 equiv) in THE (40 mL) was added t-BuONa (2 M, 7.99 mL, 1.1 equiv) and 2,2,2-trifluoroethanol (1.45 g, 1.0 equiv) at −40° C. The mixture was stirred at −40° C. for 1.5 hours. The mixture was poured into water (20 mL) and filtered. The filtrate was extracted with ethyl acetate (3×40 mL). The organic phase was dried over Na₂SO₄ and concentrated under vacuum. The crude product was purified by column chromatography afford the title compound (5.4 g, 94% yield) as yellow solid; LCMS (ESI, M+1): m/z=394.8.

Step B. 7-bromo-6-chloro-8-fluoro-2-(((2R,7aS)-2-fluorotetrahydro-1H-pyrrolizin-7a(5H)-yl)methoxy)-4-(2,2,2-trifluoroethoxy)quinazoline: To a solution of 7-bromo-2,6-dichloro-8-fluoro-4-(2,2,2-trifluoroethoxy)quinazoline (1 g, 1.0 equiv) in DMF (10 mL) were added DIEA (984 mg, 1.33 mL, 3.0 equiv), 4 Å molecular sieve (100 mg) and ((2R,7aS)-2-fluorotetrahydro-1H-pyrrolizin-7a(5H)-yl)methanol (485 mg, 1.2 equiv). The mixture was stirred at 60° C. for 12 hours. The mixture was poured into water (20 mL) and filtered. The filtrate was extracted with ethyl acetate (3×20 mL). The organic phase was dried over Na₂SO₄ and concentrated under vacuum. The crude product was purified with reversed phase flash (C18, 0.1% formic acid condition) afford the title compound (310 mg, 24% yield) as yellow solid; LCMS (ESI, M+1): m/z=518.0.

Step C. 6-chloro-7-(5,6-dimethyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)quinazoline: To a solution of 7-bromo-6-chloro-8-fluoro-2-(((2R,7aS)-2-fluorotetrahydro-1H-pyrrolizin-7a(5H)-yl)methoxy)-4-(2,2,2-trifluoroethoxy)quinazoline (250 mg, 1.0 equiv) in dioxane (2.5 mL) and H₂O (0.5 mL) was added 5,6-dimethyl-1-tetrahydropyran-2-yl-4-(4,4,5,5-tetramethyl-1,3,2-dioxaborolan-2-yl)indazole (310 mg, 1.8 equiv), RuPhos Pd G3 (40.5 mg, 48.4 umol, 0.1 equiv), Cs₂CO₃ (473 mg, 3 equiv) and RuPhos (67.7 mg, 0.3 equiv). The mixture was stirred at 80° C. for 6 hours. The reaction mixture was filtered. The crude product was purified by column chromatography afford the title compound (239 mg, 74% yield) as white solid; LCMS (ESI, M+1): m/z=666.3.

Step D. (5R)-7-(6-chloro-7-(5,6-dimethyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione: To a solution of 6-chloro-7-(5,6-dimethyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)-4-(2,2,2-trifluoroethoxy)quinazoline (100 mg, 1.0 equiv) in DMF (0.8 mL) was added DIEA (58.21 mg, 3.0 equiv) and (5R)-1,3,9-triazaspiro[4.5]decane-2,4-dione (76.2 mg, 3.0 equiv). The mixture was stirred at 60° C. for 12 hours. The mixture was poured into water (3 mL) and filtered. The filtrate was extracted with ethyl acetate (2×4 mL). The organic phase was dried over Na₂SO₄ and concentrated under vacuum. The crude product was purified with reversed phase flash (C18, 0.1% formic acid condition) afford the title compound (50 mg, 45% yield) as white solid; LCMS (ESI, M+1): m/z=735.4.

Step E. (5R)-7-(6-chloro-7-(5,6-dimethyl-1H-indazol-4-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione: To a solution of (5R)-7-(6-chloro-7-(5,6-dimethyl-1-(tetrahydro-2H-pyran-2-yl)-1H-indazol-4-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione (30 mg, 1.0 equiv) in DCM (0.3 mL) was added TFA (0.5 mL). The mixture was stirred at 25° C. for 12 hours. The reaction mixture was quenched by addition saturated NaHCO₃ solution 4 mL at 0° C. The reaction mixture was extracted with ethyl acetate (3×5 mL). The combined organic layers were concentrated and purified with prep-HPLC [Waters Xbridge 150×25 mm×5 um; mobile phase: A: water (ammonia hydroxide) B: ACN; B %: 24%-54% over 9 min] afford the title compound (8.78 mg, 27% yield) as white solid; ¹H NMR (400 MHz, METHANOL-d₄) δ=8.08 (d, J=1.3 Hz, 1H), 7.48 (s, 1H), 7.43 (s, 1H), 5.38-5.22 (m, 1H), 4.38 (s, 2H), 4.24 (s, 2H), 3.64-3.61 (m, 1H), 3.55-3.47 (m, 1H), 3.26-3.15 (m, 3H), 3.03-2.94 (m, 1H), 2.50 (s, 3H), 2.38-2.19 (m, 3H), 2.12 (s, 3H), 2.09-2.03 (m, 2H), 2.01-1.88 (m, 5H); LCMS (ESI, M+1): m/z=651.2.

### Example 34

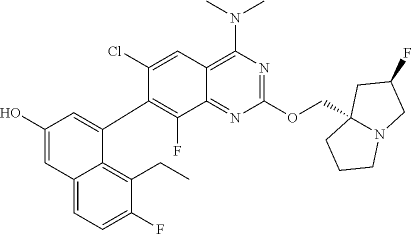 [SMILES: CCc1c(F)ccc2cc(O)cc(-c3c(Cl)cc4c(N(C)C)nc(OC[C@@]56CCCN5C[C@H](F)C6)nc4c3F)c12]

### 4-(6-chloro-4-(dimethylamino)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol

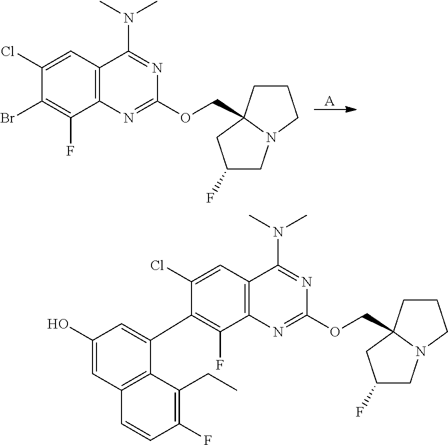

Step A. 4-(6-chloro-4-(dimethylamino)-8-fluoro-2-(((2R,7aS)-2-fluorohexahydro-1H-pyrrolizin-7a-yl)methoxy)quinazolin-7-yl)-5-ethyl-6-fluoronaphthalen-2-ol: To a mixture of 7-bromo-6-chloro-8-fluoro-2-(((2R,7aS)-2-fluorohexahydro-1H-pyrrolizin-7a-yl)methoxy)-N,N-dimethylquinazolin-4-amine (200 mg, 1.0 equiv), 5-ethyl-6-fluoro-4-(4,4,5,5-tetramethyl-1,3,2-dioxaborolan-2-yl)naphthalen-2-ol (205 mg, 1.5 equiv), Cs₂CO₃ (423 mg, 3.0 equiv) and RuPhos (60.6 mg, 0.3 equiv) in dioxane (1.5 mL) and H₂O (0.3 mL) was added RuPhos-Pd-G3 (36.2 mg, 0.1 equiv). The mixture was stirred at 90° C. for 3 hours. The mixture was diluted with water (3 mL) and extracted with EtOAc (3×10 mL). The organic layer was dried with anhydrous sodium sulfate and concentrated. The residue was purified by reversed phase flash [C18, 0.1% formic acid condition]. The crude product was purified with prep-HPLC [Phenomenex C18 75×30 mm×3 μm; A: water (FA), B: ACN; B %: 18%-48% over 7 min] and [Waters Xbridge 150×25 mm×5 μm; A: water (NH₄HCO₃), B: ACN; B %: 54%-84% over 7 min] and lyophilized to afford the title compound (21.6 mg, 8.7% yield) as yellow solid; ¹H NMR (400 MHz, METHANOL-d₄) δ=8.13 (br s, 1H), 7.73-7.57 (m, 1H), 7.34-7.12 (m, 2H), 6.90 (br s, 1H), 5.43-5.15 (m, 1H), 4.37-4.14 (m, 2H), 3.53-3.39 (m, 6H), 3.28-3.11 (m, 3H), 3.08-2.93 (m, 1H), 2.67-2.51 (m, 1H), 2.40-2.17 (m, 3H), 2.16-2.08 (m, 1H), 2.02-1.82 (m, 3H), 0.80 (br s, 3H); LCMS (ESI, M+1): m/z=571.4.

### Example 35

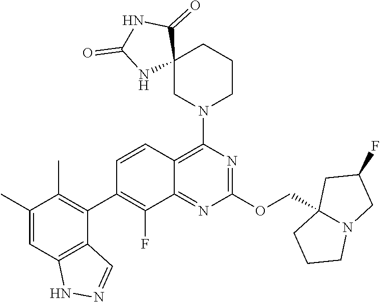 [SMILES: Cc1cc2[nH]ncc2c(-c2ccc3c(N4CCC[C@]5(C4)NC(=O)NC5=O)nc(OC[C@@]45CCCN4C[C@H](F)C5)nc3c2F)c1C]

### (5R)-7-(7-(5,6-dimethyl-1H-indazol-4-yl)-8-fluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione

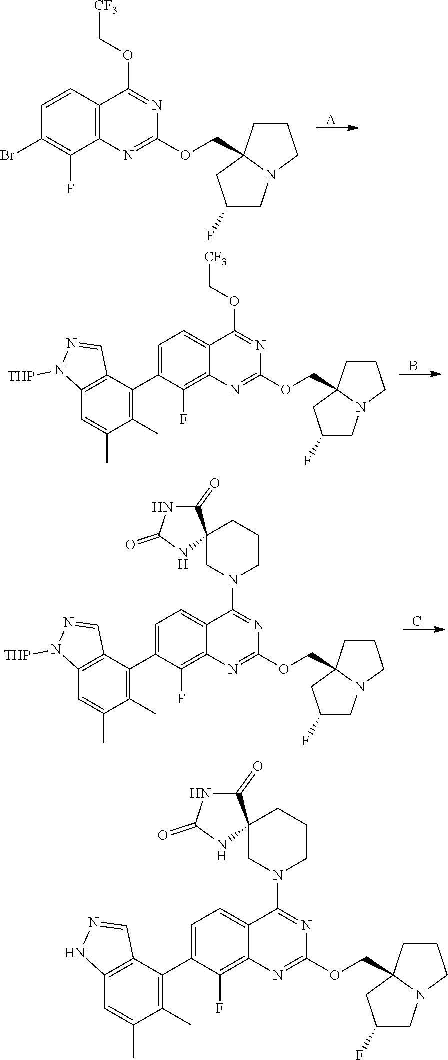

Step A. 7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)-4-(2,2,2-trifluoroethoxy)quinazoline: A mixture of 7-bromo-8-fluoro-2-(((2R,7aS)-2-fluorohexahydro-1H-pyrrolizin-7a-yl)methoxy)-4-(2,2,2-trifluoroethoxy)quinazoline (350 mg, 1.0 equiv), 5,6-dimethyl-1-(tetrahydro-2H-pyran-2-yl)-4-(4,4,5,5-tetramethyl-1,3,2-dioxaborolan-2-yl)-1H-indazole (310 mg, 1.2 equiv), Cs₂CO₃ (1 M, 2.18 mL, 3.0 equiv) in methoxycyclopentane (10 mL) was degassed and purged with N₂ for 3 times, Ad₂nBuP-Pd-G3 (52.9 mg, 0.1 equiv) was added and the mixture was stirred at 80° C. for 6 hours under N₂ atmosphere. The mixture was quenched with water (10 mL) and extracted with ethyl acetate (2×8 mL). The combined organic layers were dried over anhydrous sodium sulfate, concentrated and purified with reversed-phase flash (0.1% FA condition) to afford the title compound (350 mg, 71% yield) as white solid; LCMS (ESI, M+1): m/z=632.3.

Step B. (5R)-7-(7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)quinazolin-4-yl)-1,3,7-triazaspiro[4.5]decane-2,4-dione: To a solution of 7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)-4-(2,2,2-trifluoroethoxy)quinazoline (100 mg, 1.0 equiv), (R)-1,3,7-triazaspiro[4.5]decane-2,4-dione (40.2 mg, 1.5 equiv) and DIEA (61.4 mg, 82.7 μL, 3.0 equiv) in DMF (0.5 mL) was added 4 Å MS (25.0 mg). The mixture was stirred at 40° C. for 144 hours. The reaction mixture was filtered, washed with DMF (2 mL), and extracted with ethyl acetate (2×20 mL). The combined organic layers were dried over anhydrous sodium sulfate, concentrated and purified with prep-HPLC [column: Waters Xbridge 150×25 mm×5 μm; mobile phase: [water(NH₄HCO₃)−ACN];B %: 43%-73%, 10 min] and lyophilized to afford the title compound to afford the title compound (50.0 mg, 45% yield) as white solid; LCMS (ESI, M+1): m/z=701.4.

Step C. (5R)-7-(7-(5,6-dimethyl-1H-indazol-4-yl)-8-fluoro-2-(((2R,7aS)-2-fluorohexahydro-1H-pyrrolizin-7a-yl)methoxy)quinazolin-4-yl)-1,3,7-triazaspiro[4.5]decane-2,4-dione: To a solution of (5R)-7-(7-(5,6-dimethyl-1-(tetrahydro-2H-pyran-2-yl)-1H-indazol-4-yl)-8-fluoro-2-(((2R,7aS)-2-fluorohexahydro-1H-pyrrolizin-7a-yl)methoxy)quinazolin-4-yl)-1,3,7-triazaspiro[4.5]decane-2,4-dione (50.0 mg, 1.0 equiv) in dichloromethane (0.5 mL) was added TsOH (122 mg, 10 equiv). The mixture was stirred at 0° C. for 0.5 hour. The mixture was quenched with water (1 mL) and extracted with ethyl acetate (2×2 mL). The combined organic layers were dried over anhydrous sodium sulfate, concentrated and purified with prep-HPLC [column: Waters Xbridge 150×25 mm×5 μm; mobile phase: [water (NH₄HCO₃)−ACN]; B %: 30%-60%, 9 min] and lyophilized to afford the title compound (5.38 mg, 9.0% yield) as white solid; SFC: Chiralpak IG-3 50×4.6 mm I.D., 3 m [40% EtOH (0.05% DEA)] in CO₂, flow rate: 3 mL/min, detector: 220 nm, t_{R1}=0.737 min, t_{R2}=1.437 min; ¹H NMR (400 MHz, METHANOL-d4) δ=7.95 (d, J=8.4 Hz, 1H), 7.55-7.43 (m, 2H), 7.31 (dd, J=6.8, 8.4 Hz, 1H), 5.41 (br s, 1H), 5.28 (br s, 1H), 4.60-4.37 (m, 2H), 4.31 (s, 2H), 3.73-3.64 (m, 1H), 3.62-3.51 (m, 1H), 3.49-3.38 (m, 1H), 3.14-2.99 (m, 1H), 2.50 (s, 3H), 2.45-2.25 (m, 2H), 2.25-2.14 (m, 6H), 2.10-1.86 (m, 7H); LCMS (ESI, M+1): m/z=617.3.

### Example 36

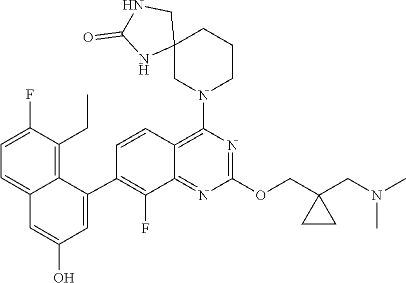 [SMILES: CCc1c(F)ccc2cc(O)cc(-c3ccc4c(N5CCCC6(CNC(=O)N6)C5)nc(OCC5(CN(C)C)CC5)nc4c3F)c12]

### 7-(2-((1-((dimethylamino)methyl)cyclopropyl)methoxy)-7-(8-ethyl-7-fluoro-3-hydroxynaphthalen-1-yl)-8-fluoroquinazolin-4-yl)-1,3,7-triazaspiro[4.5]decan-2-one

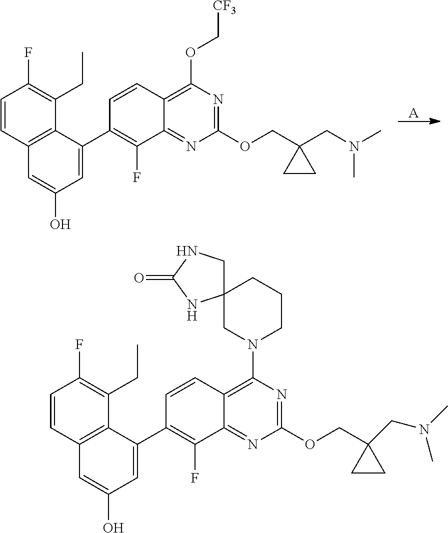

Step A. 7-(2-((1-((dimethylamino)methyl)cyclopropyl)methoxy)-7-(8-ethyl-7-fluoro-3-hydroxynaphthalen-1-yl)-8-fluoroquinazolin-4-yl)-1,3,7-triazaspiro[4.5]decan-2-one: A mixture of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (50.0 mg, 1.0 equiv), 1,3,9-triazaspiro[4.5]decan-2-one (50.0 mg, 3.6 equiv), K₃PO₄ (37.8 mg, 2.0 equiv) and 4 Å molecular sieve (25 mg) in DMF (0.5 mL) was degassed and purged with N₂ for 3 times, and then the mixture was stirred at 60° C. for 16 hours under N₂ atmosphere. The reaction mixture was cooled to room temperature. Ethyl acetate (40 mL) and water (40 mL) were added and layers were separated. The aqueous phase was extracted with ethyl acetate (2×30 mL). Combined extracts were washed with brine (40 mL), dried over sodium sulfate and filtered. The filtrate was concentrated under reduced pressure to give a residue. The residue was purified by reversed phase column (column: Phenomenex Synergi C18 150×25 mm×10 um; mobile phase: [water(FA)−ACN]; B %: 18%-38%, 10 min) lyophilized to afford the title compound (29.0 mg, 53% yield, 0.51 FA) as white solid; ¹H NMR (400 MHz, METHANOL-d₄) δ=7.93-7.84 (m, 1H), 7.70-7.60 (m, 1H), 7.37-7.29 (m, 1H), 7.27-7.19 (m, 2H), 6.95 (d, J=2.0 Hz, 1H), 4.43-4.34 (m, 2H), 4.00-3.74 (m, 4H), 3.46-3.35 (m, 1H), 3.30-3.27 (m, 1H), 2.82 (br s, 2H), 2.72-2.52 (m, 6H), 2.49-2.35 (m, 2H), 2.05-1.82 (m, 4H), 0.84 (s, 2H), 0.77 (q, J=7.2 Hz, 3H), 0.66 (s, 2H); LCMS (ESI, M+1): m/z=617.3

### Example 37

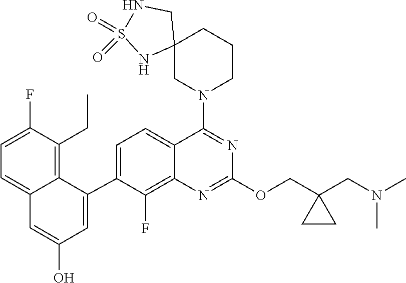 [SMILES: CCc1c(F)ccc2cc(O)cc(-c3ccc4c(N5CCCC6(CNS(=O)(=O)N6)C5)nc(OCC5(CN(C)C)CC5)nc4c3F)c12]

### 7-(2-((1-((dimethylamino)methyl)cyclopropyl)methoxy)-7-(8-ethyl-7-fluoro-3-hydroxynaphthalen-1-yl)-8-fluoroquinazolin-4-yl)-2-thia-1,3,7-triazaspiro[4.5]decane 2,2-dioxide

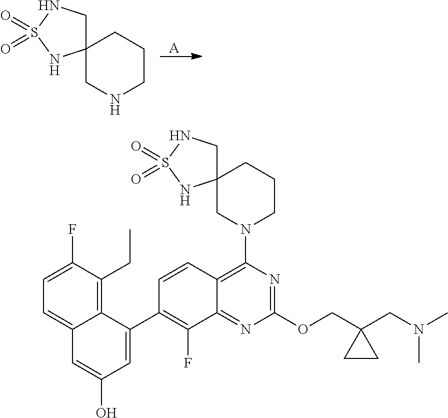

Step A. 7-(2-((1-((dimethylamino)methyl)cyclopropyl)methoxy)-7-(8-ethyl-7-fluoro-3-hydroxynaphthalen-1-yl)-8-fluoroquinazolin-4-yl)-2-thia-1,3,7-triazaspiro[4.5]decane 2,2-dioxide: To a mixture of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (10.0 mg, 1.0 equiv), 2 λ6-thia-1,3,9-triazaspiro[4.5]decane 2,2-dioxide (10.2 mg, 3.0 equiv) and 4 Å molecular sieve (10.0 mg, 1.0 equiv) in DMF (0.5 mL) was added K₃PO₄ (11.34 mg, 3.0 equiv)). The mixture was stirred at 60° C. for 12 hours. The reaction mixture was filtered and purified by reversed phase flash [C18, 0.1% formic acid condition] to afford the title compound (77.8 mg, 67% yield, 0.95 FA) as yellow solid; ¹H NMR (400 MHz, METHANOL-d₄) δ=7.96 (br d, J=8.4 Hz, 1H), 7.73-7.60 (m, 1H), 7.38-7.32 (m, 1H), 7.27-7.20 (m, 2H), 6.96-6.93 (m, 1H), 4.53-4.33 (m, 3H), 4.23 (br t, J=14.4 Hz, 1H), 3.81-3.58 (m, 2H), 3.42 (d, J=11.6 Hz, 1H), 3.27-3.12 (m, 3H), 2.89 (s, 6H), 2.56-2.37 (m, 2H), 2.14-1.98 (m, 2H), 1.97-1.81 (m, 2H), 0.95 (s, 2H), 0.85-0.72 (m, 5H); LCMS (ESI, M+1): m/z=653.3

### Example 38

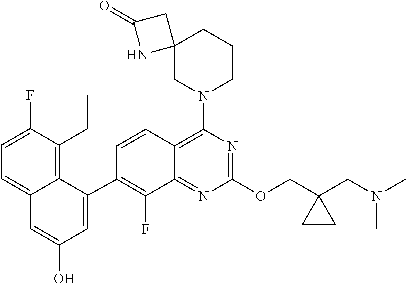 [SMILES: CCc1c(F)ccc2cc(O)cc(-c3ccc4c(N5CCCC6(CC(=O)N6)C5)nc(OCC5(CN(C)C)CC5)nc4c3F)c12]

### 6-(2-((1-((dimethylamino)methyl)cyclopropyl)methoxy)-7-(8-ethyl-7-fluoro-3-hydroxynaphthalen-1-yl)-8-fluoroquinazolin-4-yl)-1,6-diazaspiro[3.5]nonan-2-one

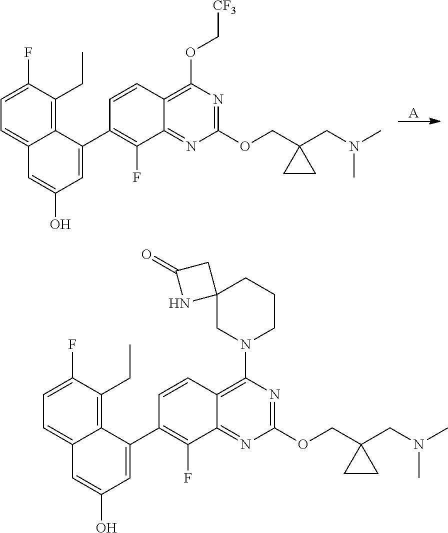

Step A. 8-[2-[[1-[(dimethylamino)methyl]cyclopropyl]methoxy]-7-(8-ethyl-7-fluoro-3-hydroxy-1-naphthyl)-8-fluoro-quinazolin-4-yl]-1,8-diazaspiro[3.5]nonan-2-one: To a solution of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (50.0 mg, 1 equiv) and 1,6-diazaspiro[3.5]nonan-2-one (37.4 mg, 3.0 equiv) in DMF (0.1 mL) and MeCN (0.1 mL) was added K₃PO₄ (56.7 mg, 3.0 equiv). The mixture was stirred at 40° C. for 12 hours. The mixture was quenched with water (3 mL) and extracted with ethyl acetate (3×2 mL). The combined organic layers were washed with brine (3×2 mL), dried over anhydrous sodium sulfate, concentrated and purified with prep-HPLC [column: Waters Xbridge 150×25 mm×5 m; mobile phase: [water(NH₄HCO₃)−ACN];B %: 36%-66%,9 min] and lyophilized to afford the title compound (25.3 mg, 46% yield) as yellow solid. SFC: Chiralcel OD-3 50×4.6 mm I.D., 3 m [MeOH (0.05% DEA) in CO₂ from 5% to 40%)] in CO₂, flow rate: 3 mL/min, detector: 220 nm, t_{R1}: 2.029 min, t_{R2}: 2.124 min, t_{R3}: 2.254 min; ¹H NMR (400 MHz, METHANOL-d4) δ=7.84 (d, J=8.4 Hz, 1H), 7.65 (dd, J=6.4, 9.2 Hz, 1H), 7.36-7.16 (m, 3H), 6.95 (d, J=2.8 Hz, 1H), 4.36 (t, J=3.6 Hz, 2H), 4.22-4.02 (m, 2H), 3.96-3.82 (m, 1H), 3.76-3.56 (m, 1H), 2.92-2.72 (m, 2H), 2.56-2.36 (m, 4H), 2.32 (s, 6H), 2.12-1.92 (m, 4H), 0.86-0.66 (m, 5H), 0.56-0.46 (m, 2H); LCMS (ESI, M+1): m/z=602.3.

### Example 39

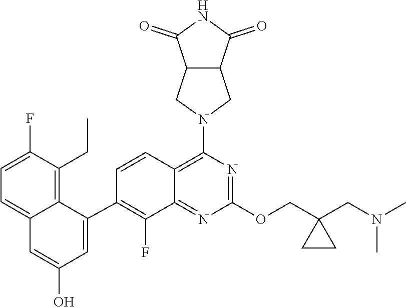 [SMILES: CCc1c(F)ccc2cc(O)cc(-c3ccc4c(N5CC6C(=O)NC(=O)C6C5)nc(OCC5(CN(C)C)CC5)nc4c3F)c12]

### 5-(2-((1-((dimethylamino)methyl)cyclopropyl)methoxy)-7-(8-ethyl-7-fluoro-3-hydroxynaphthalen-1-yl)-8-fluoroquinazolin-4-yl)tetrahydropyrrolo[3,4-c]pyrrole-1,3(2H,3aH)-dione

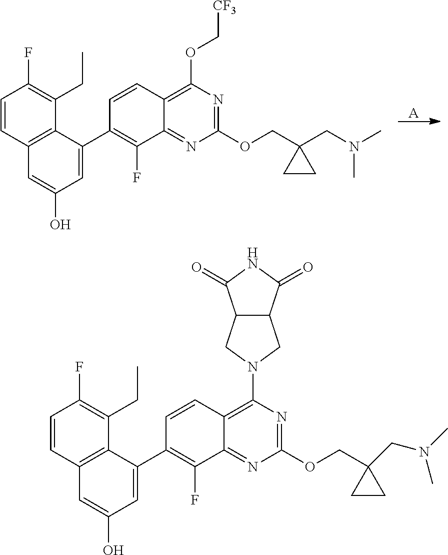

Step A. 5-(2-((1-((dimethylamino)methyl)cyclopropyl)methoxy)-7-(8-ethyl-7-fluoro-3-hydroxynaphthalen-1-yl)-8-fluoroquinazolin-4-yl)tetrahydropyrrolo[3,4-c]pyrrole-1,3(2H,3aH)-dione: To a solution of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (50.0 mg, 1.0 equiv) in DMF (0.5 mL) was added 2,3,3a,6a-tetrahydro-1H-pyrrolo[3,4-c]pyrrole-4,6-dione (25.0 mg, 2.0 equiv), ACN (0.5 mL) and K₃PO₄ (56.7 mg, 3.0 equiv). The mixture was stirred at 60° C. for 24 hours. The reaction mixture was filtered and purified by prep-HPLC (column: Phenomenex luna C18 150×25 mm×10 um; mobile phase: [water(FA)−ACN]; B %: 12%-42%, 10 min) to afford the title compound (36.5 mg, 30.8% yield) as white solid; ¹H NMR (400 MHz, METHANOL-d₄) δ=8.07 (br d, J=8.8 Hz, 1H), 7.65 (t, J=6.8 Hz, 1H), 7.36 (br t, J=8.0 Hz, 1H), 7.28-7.20 (m, 2H), 6.96 (s, 1H), 4.76-4.61 (m, 2H), 4.38 (s, 2H), 4.25-4.12 (m, 2H), 3.68 (br d, J=7.2 Hz, 2H), 2.99 (s, 2H), 2.84-2.68 (m, 6H), 2.49-2.36 (m, 2H), 0.89 (s, 2H), 0.82-0.71 (m, 5H); LCMS (ESI, M+1): m/z=602.4.

### Example 40

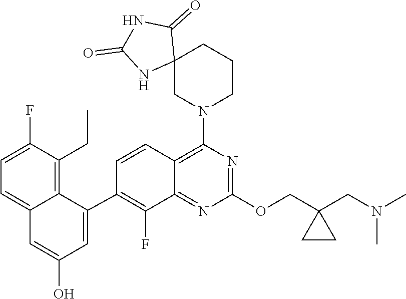 [SMILES: CCc1c(F)ccc2cc(O)cc(-c3ccc4c(N5CCCC6(C5)NC(=O)NC6=O)nc(OCC5(CN(C)C)CC5)nc4c3F)c12]

### 7-(2-((1-((dimethylamino)methyl)cyclopropyl)methoxy)-7-(8-ethyl-7-fluoro-3-hydroxynaphthalen-1-yl)-8-fluoroquinazolin-4-yl)-1,3,7-triazaspiro[4.5]decane-2,4-dione

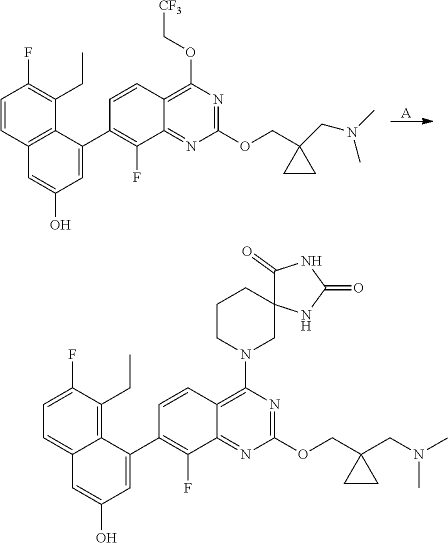

Step A. 7-(2-((1-((dimethylamino)methyl)cyclopropyl)methoxy)-7-(8-ethyl-7-fluoro-3-hydroxynaphthalen-1-yl)-8-fluoroquinazolin-4-yl)-1,3,7-triazaspiro[4.5]decane-2,4-dione: To a mixture of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (100 mg, 1.0 equiv), 1,3,9-triazaspiro[4.5]decane-2,4-dione (60.3 mg, 2.0 equiv) and 4 Å molecular sieve (10 mg) in DMF (0.5 mL) was added DIEA (69.0 mg, 3.0 equiv). The mixture was stirred at 60° C. for 24 hours. The residue was filtered and washed with DMF (1 mL) and purified with prep-HPLC [Phenomenex C18 75×30 mm×3 μm; A: water (FA), B: ACN; B %: 15%-45% over 7 min] and lyophilized to afford the title compound (54.6 mg, 47% yield) as yellow solid; ¹H NMR (400 MHz, METHANOL-d₄) δ=7.88 (d, J=8.8 Hz, 1H), 7.65 (dd, J=6.0, 9.2 Hz, 1H), 7.37-7.29 (m, 1H), 7.28-7.17 (m, 2H), 6.96 (d, J=2.4 Hz, 1H), 4.51-4.22 (m, 4H), 3.71 (dd, J=13.6, 15.2 Hz, 1H), 3.65-3.47 (m, 1H), 3.10-2.90 (m, 2H), 2.85-2.63 (m, 6H), 2.53-2.33 (m, 2H), 2.28-2.13 (m, 1H), 2.11-1.86 (m, 3H), 0.89 (s, 2H), 0.81-0.67 (m, 5H); LCMS (ESI, M+1): m/z=631.3.

### Example 41

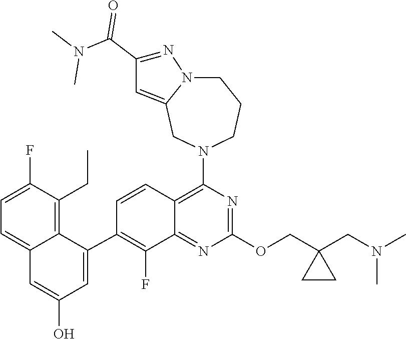 [SMILES: CCc1c(F)ccc2cc(O)cc(-c3ccc4c(N5CCCn6nc(C(=O)N(C)C)cc6C5)nc(OCC5(CN(C)C)CC5)nc4c3F)c12]

### 5-(2-((1-((dimethylamino)methyl)cyclopropyl)methoxy)-7-(8-ethyl-7-fluoro-3-hydroxynaphthalen-1-yl)-8-fluoroquinazolin-4-yl)-N,N-dimethyl-5,6,7,8-tetrahydro-4H-pyrazolo[1,5-a][1,4]diazepine-2-carboxamide

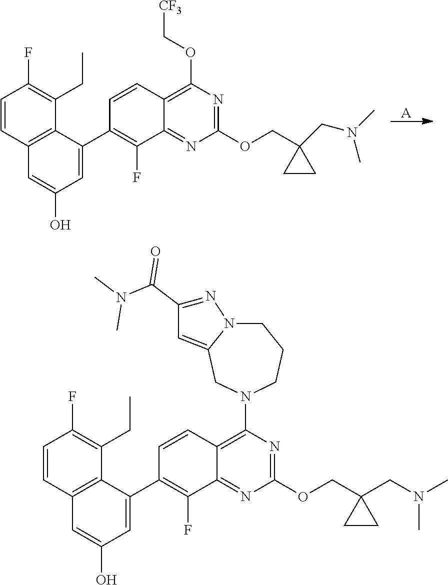

Step A. 5-(2-((1-((dimethylamino)methyl)cyclopropyl)methoxy)-7-(8-ethyl-7-fluoro-3-hydroxynaphthalen-1-yl)-8-fluoroquinazolin-4-yl)-N,N-dimethyl-5,6,7,8-tetrahydro-4H-pyrazolo[1,5-a][1,4]diazepine-2-carboxamide: To a mixture of 4-(2-((1-((dimethylamino)methyl)cyclopropyl)methoxy)-8-fluoro-4-(2,2,2-trifluoroethoxy)quinazolin-7-yl)-5-ethyl-6-fluoronaphthalen-2-ol (150 mg, 1.0 equiv), N,N-dimethyl-5,6,7,8-tetrahydro-4H-pyrazolo[1,5-a][1,4]diazepine-2-carboxamide (167 mg, 3.0 equiv) and 4 Å molecular sieve (50 mg) in DMF (0.05 mL) was added DIEA (103 mg, 3.0 equiv). The mixture was stirred at 100° C. for 24 hours. The residue was filtered and washed with DMF (1 mL) and purified with prep-HPLC [Phenomenex C18 75×30 mm×3 μm; A: water (FA), B: ACN; B %: 18%-48% over 7 min] to give a crude product. The crude product was purified with prep-HPLC [Waters Xbridge 150×25 mm×5 μm; A: water (NH₄HCO₃), B: ACN; B %: 35%-65% over 9 min] and lyophilized to afford the title compound (23.2 mg, 13% yield) as white solid; ¹H NMR (400 MHz, METHANOL-d4) 6=7.91 (d, J=8.8 Hz, 1H), 7.64 (dd, J=6.0, 9.2 Hz, 1H), 7.30 (dd, J=6.8, 8.8 Hz, 1H), 7.26-7.16 (m, 2H), 6.94 (d, J=2.4 Hz, 1H), 6.67 (s, 1H), 5.27-5.03 (m, 2H), 4.60-4.48 (m, 2H), 4.40-4.24 (m, 4H), 3.34 (s, 3H), 3.08 (s, 3H), 2.52-2.43 (m, 3H), 2.42-2.35 (m, 3H), 2.31 (s, 6H), 0.76 (t, J=7.2 Hz, 3H), 0.73-0.66 (m, 2H), 0.58-0.46 (m, 2H); LCMS (ESI, M+1): m/z=670.4.

### Example 42

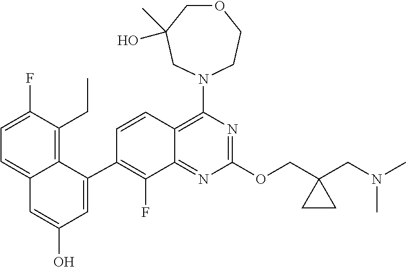 [SMILES: CCc1c(F)ccc2cc(O)cc(-c3ccc4c(N5CCOCC(C)(O)C5)nc(OCC5(CN(C)C)CC5)nc4c3F)c12]

### 4-(2-((1-((dimethylamino)methyl)cyclopropyl)methoxy)-7-(8-ethyl-7-fluoro-3-hydroxynaphthalen-1-yl)-8-fluoroquinazolin-4-yl)-6-methyl-1,4-oxazepan-6-ol

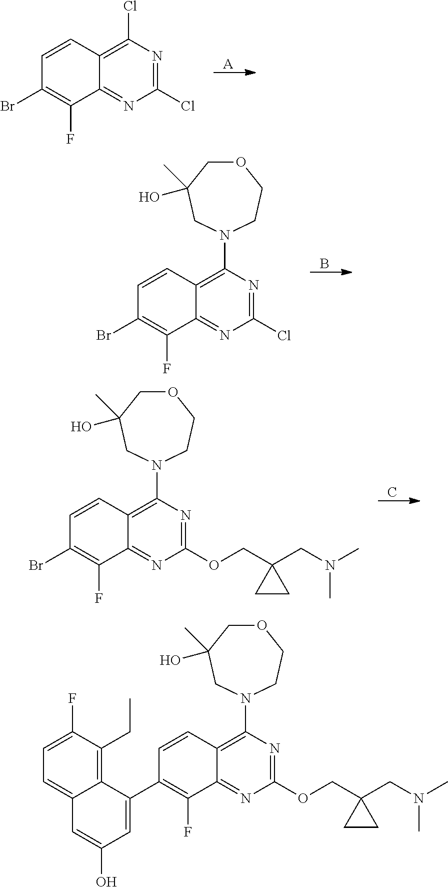

Step A. 4-(7-bromo-2-chloro-8-fluoroquinazolin-4-yl)-6-methyl-1,4-oxazepan-6-ol: To a solution of 7-bromo-2,4-dichloro-8-fluoroquinazoline (500 mg, 1.0 equiv) and 6-methyl-1,4-oxazepan-6-ol (199 mg, 0.9 equiv) in dichloromethane (5 mL) was added DIEA (655 mg, 3 equiv). The mixture was stirred at 0-20° C. for 16 hour. The reaction mixture was quenched by addition of water (10 mL) and was extracted with ethyl acetate (3×10 mL). The combined organic layers were washed with saturated brine (3×10 mL), dried over anhydrousNa₂SO₄, filtered and concentrated under reduced pressure to dryness. The crude product was purified by reversed-phase flash (0.1% FA condition) to afford the title compound (150 mg, 41% yield) as yellow solid; Column: Chiralcel OD-3 50×4.6 mm I.D., 3 m [MeOH (0.05% DEA) in CO₂ from 5% to 40%)] in CO₂, flow rate: 3 mL/min, detector: 220 nm, t_{R1}: 2.029 min, t_{R2}: 2.124 min, t_{R3}: 2.254 min; ¹H NMR (400 MHz, DMSO-d₆) δ=8.24-8.14 (m, 1H), 7.78-7.63 (m, 1H), 5.09 (s, 1H), 4.25-4.10 (m, 2H), 4.07-3.88 (m, 3H), 3.78 (ddd, J=3.2, 6.8, 14.4 Hz, 1H), 3.57-3.48 (m, 2H), 1.11 (s, 3H); LCMS (ESI, M+1): m/z=391.9.

Step B. 4-(7-bromo-2-((1-((dimethylamino)methyl)cyclopropyl)methoxy)-8-fluoroquinazolin-4-yl)-6-methyl-1,4-oxazepan-6-ol: To solution of 4-(7-bromo-2-chloro-8-fluoroquinazolin-4-yl)-6-methyl-1,4-oxazepan-6-ol (270 mg, 1.0 equiv) in DMSO (3 mL) was added (1-((dimethylamino)methyl)cyclopropyl)methanol (179 mg, 2.0 equiv), DIEA (268 mg, 3 equiv) and 4 Å molecular sieve (80.0 mg), and the reaction mixture was stirred at 80° C. for 36 hours. The reaction mixture was quenched by addition of water (10 mL) and was extracted with ethyl acetate (3×10 mL). The combined organic layers were washed with saturated brine (3×10 mL), dried over anhydrousNa₂SO₄, filtered and concentrated under reduced pressure to dryness. The crude product was purified by reversed-phase flash (0.1% FA condition) to afford the title compound (8.33 mg, 20% yield) as white solid; ¹H NMR (400 MHz, METHANOL-d₄) δ=9.13 (s, 1H), 7.67 (dd, J=6.0, 9.2 Hz, 1H), 7.30 (d, J=2.8 Hz, 1H), 7.24 (t, J=9.6 Hz, 1H), 7.03 (d, J=2.4 Hz, 1H), 5.42-5.22 (m, 1H), 4.41-4.24 (m, 2H), 4.02-3.90 (m, 2H), 3.27-3.13 (m, 3H), 3.05-2.98 (m, 1H), 2.71 (t, J=6.4 Hz, 2H), 2.50 (br s, 1H), 2.39-2.19 (m, 2H), 2.18-2.10 (m, 2H), 2.04-1.90 (m, 3H), 0.78 (t, J=7.2 Hz, 3H).

Step C. 4-(2-((1-((dimethylamino)methyl)cyclopropyl)methoxy)-7-(8-ethyl-7-fluoro-3-hydroxynaphthalen-1-yl)-8-fluoroquinazolin-4-yl)-6-methyl-1,4-oxazepan-6-ol: A mixture of 4-[7-bromo-2-[[1-[(dimethylamino)methyl]cyclopropyl]methoxy]-8-fluoro-quinazolin-4-yl]-6-methyl-1,4-oxazepan-6-ol (130 mg, 1.0 equiv), 5-ethyl-6-fluoro-4-(4,4,5,5-tetramethyl-1,3,2-dioxaborolan-2-yl)naphthalen-2-ol (128 mg, 1.5 equiv), Ad₂nBuP-Pd-G3 (39.2 mg, 0.2 equiv), Cs₂CO₃ (1 M, 807 μL, 3.0 equiv) in CPME (1 mL) was degassed and purged with N₂ for 3 times, and then the mixture was stirred at 90° C. for 2 hour under N₂ atmosphere. The reaction mixture was quenched by addition of water (10 mL) and was extracted with ethyl acetate (3×10 mL). The combined organic layer was washed with saturated brine (3×10 mL), dried over anhydrousNa₂SO₄, filtered and concentrated under reduced pressure to dryness. The residue was purified by prep-HPLC (column: Phenomenex luna C18 150*25 mm*10 m; mobile phase: [water(FA)−ACN];B %: 16%-46%, 10 min) to afford the title compound (150 mg, 41% yield) as yellow solid; ¹H NMR (400 MHz, METHANOL-d₄) δ=8.60-8.49 (m, 1H), 8.25-8.13 (m, 1H), 7.72-7.61 (m, 1H), 7.39-7.30 (m, 1H), 7.28-7.19 (m, 2H), 7.00-6.93 (m, 1H), 4.53-4.31 (m, 4H), 4.18-3.88 (m, 4H), 3.75-3.60 (m, 2H), 3.18-2.97 (m, 2H), 2.82 (s, 6H), 2.56-2.37 (m, 2H), 1.30-1.21 (m, 3H), 0.95-0.88 (m, 2H), 0.83-0.73 (m, 5H); LCMS (ESI, M+1): m/z=593.4.

### Example 43

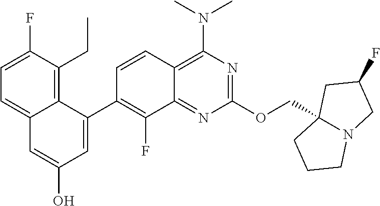 [SMILES: CCc1c(F)ccc2cc(O)cc(-c3ccc4c(N(C)C)nc(OC[C@@]56CCCN5C[C@H](F)C6)nc4c3F)c12]

### 4-(4-(dimethylamino)-8-fluoro-2-(((2R,7aS)-2-fluorotetrahydro-1-pyrrolizin-7a(5H)-yl)methoxy)quinazolin-7-yl)-5-ethyl-6-fluoronaphthalen-2-ol

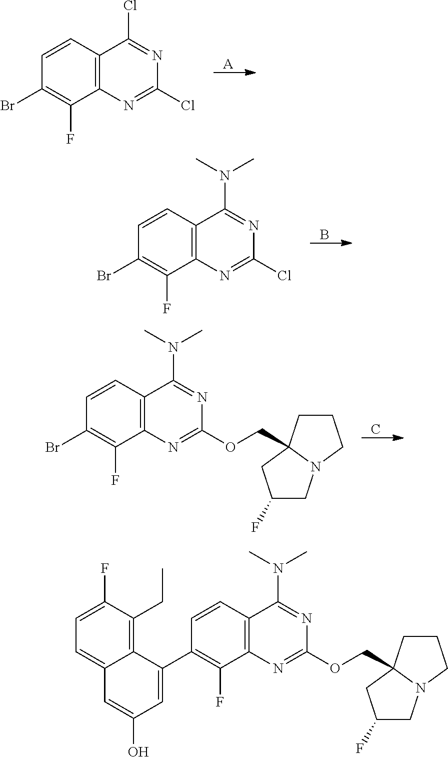

Step A. 7-bromo-2-chloro-8-fluoro-N,N-dimethylquinazolin-4-amine: To a solution of 7-bromo-2,4-dichloro-8-fluoro-quinazoline (600 mg, 1.0 equiv) and DIEA (786 mg, 3.0 equiv) in DCM (5 mL) was added N-methylmethanamine (2M in THF, 979 mg, 10 equiv) dropwise at 0° C. The mixture was stirred at 25° C. for 12 hours. The mixture was diluted with water (20 mL) and extracted with DCM (4×10 mL). The organic phase was dried over Na₂SO₄, concentrated and purified with reversed phase flash [C18, 0.1% FA] to afford the title compound (320 mg, 47% yield) as yellow solid; LCMS (ESI, M+1): m/z=305.9.

Step B. 7-bromo-8-fluoro-2-(((2R,7aS)-2-fluorohexahydro-1H-pyrrolizin-7a-yl)methoxy)-N,N-dimethylquinazolin-4-amine: To a solution of 7-bromo-2-chloro-8-fluoro-N,N-dimethyl-quinazolin-4-amine (250 mg, 1.0 equiv) and ((2R,7aS)-2-fluorohexahydro-1H-pyrrolizin-7a-yl)methanol (196 mg, 1.5 equiv) in dioxane (2 mL) was added DIEA (265 mg, 2.5 equiv) and 4 Å molecular sieve (25 mg). The mixture was stirred at 100° C. for 120 hours. The reaction mixture was filtered and purified with reversed phase flash [C18, 0.1% FA] to afford the title compound (180 mg, 49% yield) as yellow solid; LCMS (ESI, M+1): m/z=427.1.

Step C. 4-(4-(dimethylamino)-8-fluoro-2-(((2R,7aS)-2-fluorohexahydro-1H-pyrrolizin-7a-yl)methoxy)quinazolin-7-yl)-5-ethyl-6-fluoronaphthalen-2-ol: To a mixture of 7-bromo-8-fluoro-2-(((2R,7aS)-2-fluorohexahydro-1H-pyrrolizin-7a-yl)methoxy)-N,N-dimethylquinazolin-4-amine (150 mg, 1.0 equiv), 5-ethyl-6-fluoro-4-(4,4,5,5-tetramethyl-1,3,2-dioxaborolan-2-yl)naphthalen-2-ol (166 mg, 1.5 equiv) and K₃PO₄ (1.5 M in H₂O, 3.0 equiv) in methoxycyclopentane (1.5 mL) was added Ad2nBuP-Pd-G3 (50 mg, 0.1 equiv) under N₂ atmosphere. The mixture was stirred at 90° C. for 1 hour under N₂ atmosphere. The mixture was diluted with water (20 mL) and extracted with ethyl acetate (2×20 mL). The organic phase was dried over Na₂SO₄ and concentrated. The crude product was purified with reversed phase flash [C18, 0.1% FA] and prep-HPLC [Phenomenex luna C18 150×25 mm×10 μm; mobile phase: water(FA)-CAN; B %: 15%-45%, 10 minutes] to afford the title compound (91.21 mg, 45.51% yield, 0.61 FA) as white solid; ¹H NMR (400 MHz, METHANOL-d₄) δ=8.06 (br d, J=8.6 Hz, 1H), 7.64 (br dd, J=6.0, 8.8 Hz, 1H), 7.29-7.22 (m, 1H), 7.41-7.10 (m, 2H), 6.95 (s, 1H), 5.58-5.34 (m, 1H), 4.66-4.37 (m, 2H), 3.84-3.60 (m, 3H), 3.50 (s, 6H), 3.31-3.12 (m, 1H), 2.63-2.02 (m, 8H), 0.77 (br t, J=7.2 Hz, 3H); LCMS (ESI, M+1): m/z=537.2.

### Example 44

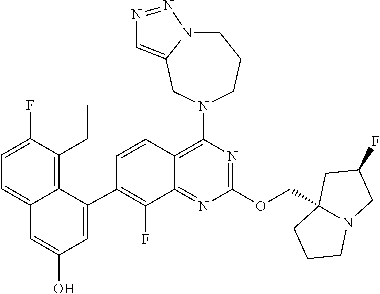 [SMILES: CCc1c(F)ccc2cc(O)cc(-c3ccc4c(N5CCCn6nncc6C5)nc(OC[C@@]56CCCN5C[C@H](F)C6)nc4c3F)c12]

### 4-4(4-(7,8-dihydro-4H-[1,2,3]triazolo[1,5-a][1,4]diazepin-5(6H)-yl)-8-fluoro-2-(((2R,7aS-2-fluorotetrahydro-1H-pyrrolizin-7a(5H)-yl)methoxy)quinazolin-7-yl)-5-ethyl-6-fluoronaphthalen-2-ol

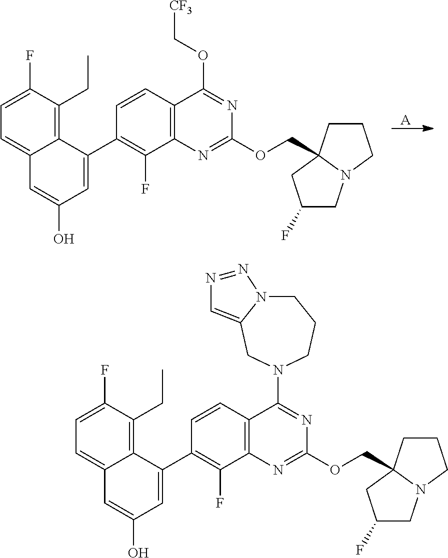

Step A. 4-(4-(7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-5-ethyl-6-fluoronaphthalen-2-ol: To a solution of 5-ethyl-6-fluoro-4-(8-fluoro-2-(((2R,7aS)-2-fluorotetrahydro-1H-pyrrolizin-7a(5H)-yl)methoxy)-4-(2,2,2-trifluoroethoxy)quinazolin-7-yl)naphthalen-2-ol (100 mg, 1.0 equiv) and 5,6,7,8-tetrahydro-4H-triazolo[1,5-a][1,4]diazepine (59.0 mg, 2.0 equiv, HCl) in DMF (0.1 mL) and ACN (0.1 mL) was added K₃PO₄ (215 mg, 6.0 equiv). The mixture was stirred at 40° C. for 72 hours. The mixture was poured into water (2 mL) and filtered. The filtrate was extracted with ethyl acetate (3×10 mL). The organic phase was dried over Na₂SO₄ and concentrated under vacuum. The crude product was purified with prep-HPLC [Phenomenex Gemini-NX C18 150×25 mm×10 μm; A: (water(FA)), B: ACN]; B %: 15%-45% over 10 min) to afford the title compound (10 mg, 9.1% yield) as yellow solid; ¹H NMR (400 MHz, METHANOL-d₄) δ=8.51 (s, 1H), 8.02-7.90 (m, 1H), 7.72-7.58 (m, 1H), 7.44-7.29 (m, 1H), 7.29-7.19 (m, 2H), 7.19-7.18 (m, 1H), 6.93 (dd, J=2.4, 7.2 Hz, 1H), 5.54-5.14 (m, 2H), 4.83-4.72 (m, 1H), 4.49-4.29 (m, 3H), 3.66-3.32 (m, 5H), 3.27-3.09 (m, 1H), 2.71-1.83 (m, 10H), 0.77 (q, J=7.2 Hz, 3H); LCMS (ESI, M+1): m/z=630.2.

### Example 45

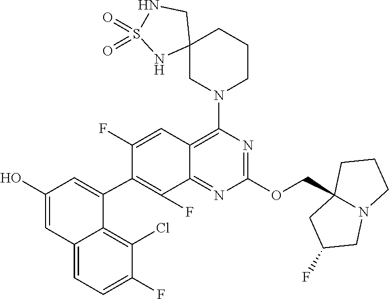 [SMILES: O=S1(=O)NCC2(CCCN(c3nc(OC[C@@]45CCCN4C[C@H](F)C5)nc4c(F)c(-c5cc(O)cc6ccc(F)c(Cl)c56)c(F)cc34)C2)N1]

### 7-(7-(8-chloro-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2-thia-1,3,7-triazaspiro[4.5]decane 2,2-dioxide

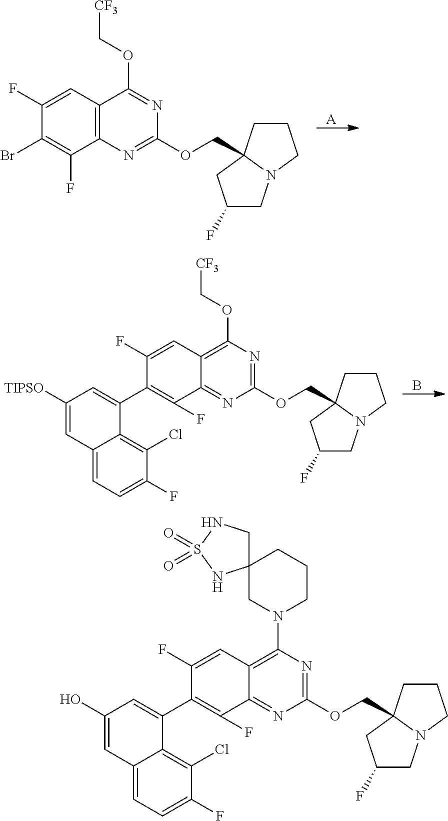

Step A. 7-(8-chloro-7-fluoro-3-((triisopropylsilyl)oxy)naphthalen-1-yl)-6,8-difluoro-2-(((2R,7aS)-2-fluorohexahydro-1H-pyrrolizin-7a-yl)methoxy)-4-(2,2,2-trifluoroethoxy)quinazoline: A mixture of 7-bromo-6,8-difluoro-2-(((2R,7aS)-2-fluorohexahydro-1H-pyrrolizin-7a-yl)methoxy)-4-(2,2,2-trifluoroethoxy)quinazoline (350 mg, 1.0 equiv), (5-chloro-6-fluoro-4-trimethylstannyl-2-naphthyl)oxy-triisopropyl-silane (433 mg, 1.2 eq), CuI (40.0 mg, 0.3 equiv) and BINAP (87.1 mg, 0.2 equiv) in toluene (3 mL) was degassed and purged with N₂ for 3 times. Pd(dppf)Cl₂ (51.2 mg, 0.1 equiv) was added and the mixture was stirred at 90° C. for 12 hours under N₂ atmosphere. The mixture was filtered. The filtrate was diluted with water (20 mL) and extracted with ethyl acetate (2×20 mL). The organic phase was dried over Na₂SO₄, concentrated and purified with reversed phase flash [C18, 0.1% FA] to afford the tittle compound (130 mg, 16% yield) as yellow solid; LCMS (ESI, M+1, M+3): m/z=772.1, 774.1.

Step B. 7-(7-(8-chloro-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-2-thia-1,3,7-triazaspiro[4.5]decane 2,2-dioxide: To a solution of 7-(8-chloro-7-fluoro-3-((triisopropylsilyl)oxy)naphthalen-1-yl)-6,8-difluoro-2-(((2R,7aS)-2-fluorohexahydro-1H-pyrrolizin-7a-yl)methoxy)-4-(2,2,2-trifluoroethoxy)quinazoline (70.0 mg, 1.0 equiv) and 2-thia-1,3,7-triazaspiro[4.5]decane 2,2-dioxide (32.6 mg, 1.5 equiv) in DMF (0.8 mL) was added K₃PO₄ (72.4 mg, 3.0 equiv) and 4 Å molecular sieve (20 mg). The mixture was stirred at 60° C. for 12 hours. The mixture was filtered and purified with prep-HPLC [Phenomenex Synergi C18 150×25 mm×10 μm; mobile phase: water(FA)−ACN; B %: 20%-40%, 10 minutes] to afford the title compound (19.6 mg, 23% yield, 0.42 FA) as yellow solid; ¹H NMR (400 MHz, methanol-d₄) δ=7.87-7.75 (m, 1H), 7.66 (br dd, J=2.8, 9.6 Hz, 1H), 7.44-7.32 (m, 2H), 7.15-7.11 (m, 1H), 5.55-5.31 (m, 1H), 4.57-4.41 (m, 2H), 4.40-4.27 (m, 1H), 4.27-4.10 (m, 1H), 3.77-3.46 (m, 6H), 3.42 (br d, J=11.2 Hz, 1H), 3.22 (br d, J=11.2 Hz, 2H), 2.65-2.24 (m, 4H), 2.16 (br s, 2H), 2.05 (br d, J=9.2 Hz, 3H), 1.95-1.78 (m, 2H); LCMS (ESI, M+1): m/z=707.0

### Example 46

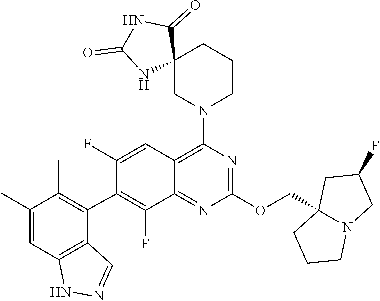 [SMILES: Cc1cc2[nH]ncc2c(-c2c(F)cc3c(N4CCC[C@]5(C4)NC(=O)NC5=O)nc(OC[C@@]45CCCN4C[C@H](F)C5)nc3c2F)c1C]

### (5R)-7-(7-(5,6-dimethyl-1H-indazol-4-yl)-6,8-difluoro-2-(((2R,7aS)-2-fluorotetrahydro-1H-pyrrolizin-7a(5H)-yl)methoxy)quinazolin-4-yl)-1,3,7-tri azaspiro[4.5]decane-2,4-dione

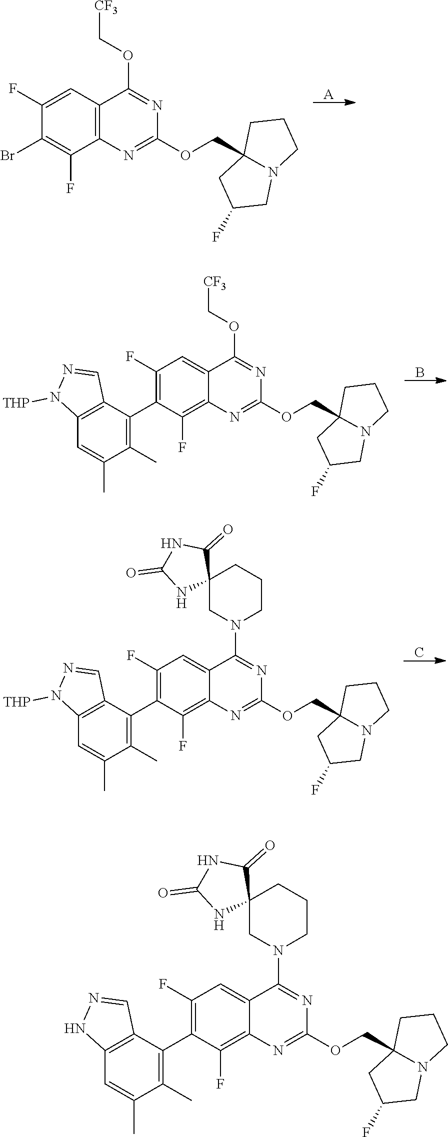

Step A. 7-(5,6-dimethyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)-4-(2,2,2-trifluoroethoxy)quinazoline: To a mixture of 7-bromo-6,8-difluoro-2-(((2R,7aS)-2-fluorotetrahydro-1H-pyrrolizin-7a(5H)-yl)methoxy)-4-(2,2,2-trifluoroethoxy)quinazoline (350 mg, 1 equiv), 5,6-dimethyl-1-(tetrahydro-2H-pyran-2-yl)-4-(4,4,5,5-tetramethyl-1,3,2-dioxaborolan-2-yl)-1H-indazole (374 mg, 1.5 equiv) in methoxycyclopentane (3.5 mL) was added Cs₂CO₃ (1.5 M, 1.40 mL, 3 equiv) at 25° C. The mixture was degassed and purged with N₂ for 3 times, and then Ad2nBuP-Pd-G3 (51.0 mg, 70.0 umol, 0.1 equiv) was added. The reaction was degassed and purged with N₂ for 3 times and stirred at 90° C. for 2 hours under N₂ atmosphere. The reaction mixture was diluted with H₂O (5 mL) and extracted with ethyl acetate (3×5 mL). The combined organic layers were dried over anhydrous Na₂SO₄, concentrated and purified with reversed-phase HPLC [0.1% FA condition] to afford the title compound (357 mg, 78% yield) as a white solid; LCMS (ESI, M+1): m/z=650.4.

Step B. (5R)-7-(7-(5,6-dimethyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)guinazolin-4-yl)-1,3,7-triazaspiro[4.5]decane-2,4-dione: To a mixture of 7-(5,6-dimethyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)-4-(2,2,2-trifluoroethoxy)quinazoline (280 mg, 1 equiv), (R)-1,3,7-triazaspiro[4.5]decane-2,4-dione (146 mg, 2 equiv) in DMF (1.4 mL) and ACN (1.4 mL) was added K₃PO₄ (274 mg, 3 equiv) and 4 Å molecular sieve (250 mg). The mixture was degassed and purged with N₂ for 3 times, and stirred at 60° C. for 12 hours under N₂ atmosphere. The reaction mixture was filtered and concentrated under reduced pressure. The residue was purified with reversed-phase HPLC [0.1% NH₃H₂O] to afford the title compound (140 mg, 45% yield) as a yellow solid; ¹H NMR (400 MHz, methanol-d₄) δ=7.71 (d, J=9.6 Hz, 1H), 7.65 (s, 1H), 7.52 (s, 1H), 5.82 (br d, J=8.4 Hz, 1H), 5.43-5.18 (m, 1H), 4.41-4.29 (m, 2H), 4.25 (s, 2H), 4.03 (br d, J=11.6 Hz, 1H), 3.84 (dt, J=2.4, 11.2 Hz, 1H), 3.61 (d, J=13.2 Hz, 1H), 3.52-3.41 (m, 1H), 3.28-3.16 (m, 2H), 3.07-2.95 (m, 1H), 2.53 (s, 3H), 2.52-2.44 (m, 1H), 2.41-2.09 (m, 8H), 2.08-1.83 (m, 9H), 1.81-1.59 (m, 2H); LCMS (ESI, M+1): m/z=719.5.

Step C. (5R)-7-(7-(5,6-dimethyl-1H-indazol-4-yl)-6,8-difluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione: To a solution of (5R)-7-(7-(5,6-dimethyl-1-(tetrahydro-2H-pyran-2-yl)-1H-indazol-4-yl)-6,8-difluoro-2-(((2R,7aS)-2-fluorotetrahydro-1H-pyrrolizin-7a(5H)-yl)methoxy)quinazolin-4-yl)-1,3,7-triazaspiro[4.5]decane-2,4-dione (40.0 mg, 1 equiv) in MeOH (0.4 mL) was added HCl.MeOH (4 M, 28.7 equiv) and stirred at 20° C. for 6 hours. The reaction mixture was filtered and concentrated under reduced pressure. The residue was purified with prep-HPLC [column: Waters Xbridge 150×25 mm×5 um; mobile phase: [water (ammonia hydroxide v/v)−ACN]; B %: 20%-50%, 9 minutes] to afford the title compound (14.3 mg, 39% yield) as a white solid; ¹H NMR (400 MHz, methanol-d₄) δ=7.72 (br d, J=10.0 Hz, 1H), 7.51 (d, J=10.0 Hz, 2H), 5.44-5.15 (m, 1H), 4.42-4.29 (m, 2H), 4.29-4.19 (m, 2H), 3.58 (dd, J=4.4, 13.2 Hz, 1H), 3.47-3.37 (m, 1H), 3.24-3.14 (m, 2H), 3.07-2.94 (m, 1H), 2.50 (s, 3H), 2.41-2.07 (m, 8H), 2.07-1.74 (m, 6H); LCMS (ESI, M+1): m/z=635.5.

### Example 47

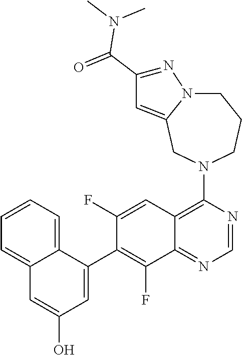 [SMILES: CN(C)C(=O)c1cc2n(n1)CCCN(c1ncnc3c(F)c(-c4cc(O)cc5ccccc45)c(F)cc13)C2]

### 5-(6,8-difluoro-7-(3-hydroxynaphthalen-1-yl)quinazolin-4-yl)-N,N-dimethyl-5,6,7,8-tetrahydro-4H-pyrazolo[1,5-a][1,4]diazepine-2-carboxamide

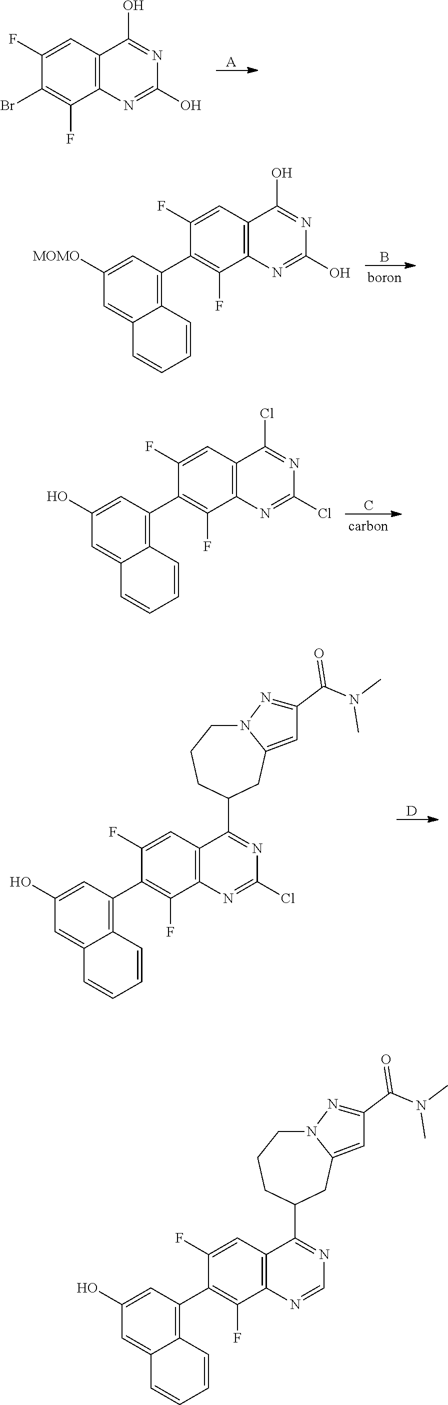

Step A. 6,8-difluoro-7-(3-(methoxymethoxy)naphthalen-1-yl)guinazolin-2,4-diol: A mixture of 2-[3-(methoxymethoxy)-1-naphthyl]-4,4,5,5-tetramethyl-1,3,2-dioxaborolane (425 mg, 1.5 equiv), 7-bromo-6,8-difluoro-quinazoline-2,4-diol (250 mg, 1.0 equiv), Cs₂CO₃ (882 mg, 3.0 equiv) and Ad₂nBuP-Pd-G3 (65.7 mg, 0.1 equiv) in ethyl alcohol (20 mL) and water (4 mL) was degassed and purged with nitrogen for 3 times, and then the mixture was stirred at 80° C. for 1.5 hours under nitrogen atmosphere. The mixture was concentrated under reduced pressure to give a residue. The residue was purified by prep-TLC (PE/EA=3/1) to afford the title compound (120 mg, 14.1% yield) as a yellow solid. ¹H NMR (400 MHz, CHLOROFORM-d) δ=7.89-7.75 (m, 2H), 7.62-7.36 (m, 5H), 5.42-5.27 (m, 2H), 3.61-3.52 (m, 3H); LCMS (ESI, M+1): m/z=385.0

Step B. 4-(2,4-dichloro-6,8-difluoroquinazolin-7-yl)naphthalen-2-ol: A mixture of 6,8-difluoro-7-[3-(methoxymethoxy)-1-naphthyl]quinazoline-2,4-diol (30.0 mg, 1.0 equiv), POCl₃ (144 mg, 12 equiv) and DIEA (40.3 mg, 4.0 equiv) were stirred at 130° C. for 0.2 hour under N₂ atmosphere. The mixture was concentrated under reduced pressure to afford the title compound (30 mg, crude) as yellow oil which was used into the next step without further purification.

Step C 5-(2-chloro-6,8-difluoro-7-(3-hydroxynaphthalen-1-yl)quinazolin-4-yl)-N,N-dimethyl-5,6,7,8-tetrahydro-4H-pyrazolo[1,5-a][1,4]diazepine-2-carboxamide: A mixture of 4-(2,4-dichloro-6,8-difluoroquinazolin-7-yl)naphthalen-2-ol (50.0 mg, 1.0 equiv), N,N-dimethyl-5,6,7,8-tetrahydro-4H-pyrazolo[1,5-a][1,4]diazepine-2-carboxamide (37.1 mg, 1.5 equiv) and DIEA (46.0 mg, 3.0 equiv) in DCM (2 mL) was degassed and purged with N₂ for 3 times, and then the mixture was stirred at −40° C. for 1 hour under N₂ atmosphere. The reaction mixture was concentrated under reduced pressure to remove solvent. The residue was purified by prep-HPLC (column: Phenomenex luna C18 150×25 mm×10 um; mobile phase: [water(FA)−ACN];B %: 39%-69%,10 min) to afford the title compound (10 mg, 15% yield) as a yellow solid; LCMS (ESI, M+1): m/z=549.4

Step D. 5-(6,8-difluoro-7-(3-hydroxynaphthalen-1-yl)quinazolin-4-yl)-N,N-dimethyl-5,6,7,8-tetrahydro-4H-pyrazolo[1,5-a][1,4]diazepine-2-carboxamide: A mixture of 5-[2-chloro-6,8-difluoro-7-(3-hydroxy-1-naphthyl)quinazolin-4-yl]-N,N-dimethyl-4,6,7,8-tetrahydropyrazolo[1,5-a][1,4]diazepine-2-carboxamide (10 mg, 1 equiv) and Pd/C (5 mg, 10% purity, 0.1 equiv) in MeOH (2 mL) was degassed and purged with H₂ for 3 times, and then the mixture was stirred at 25° C. for 1 hour under H₂ atmosphere. The mixture was filtered and concentrated under reduced pressure to give a residue. The residue was purified by prep-HPLC (column: Unisil 3-100 C18 Ultra 150×50 mm×3 um; mobile phase: [water(FA)−ACN]; B %: 28%-58%,7 min) to afford the title compound (1.08 mg, 11% yield) as an off-white solid; ¹H NMR (400 MHz, METHANOL-d₄) δ=8.64 (s, 1H), 7.83-7.75 (m, 2H), 7.47-7.40 (m, 1H), 7.35 (br d, J=8.8 Hz, 1H), 7.30 (d, J=2.0 Hz, 1H), 7.27-7.20 (m, 1H), 7.15 (d, J=2.4 Hz, 1H), 6.70 (s, 1H), 5.18 (s, 2H), 4.59 (s, 4H), 3.35 (s, 2H), 3.34 (s, 3H), 3.08 (s, 3H); LCMS (ESI, M+1): m/z=515.3

### Example 48

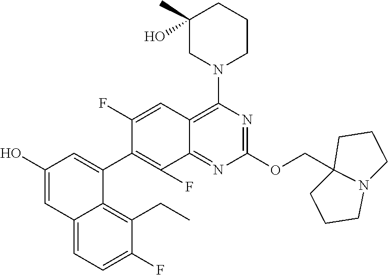 [SMILES: CCc1c(F)ccc2cc(O)cc(-c3c(F)cc4c(N5CCC[C@@](C)(O)C5)nc(OCC56CCCN5CCC6)nc4c3F)c12]

### (3R)-1-(7-(8-ethyl-7-fluoro-3-hydroxynaphthalen-1-yl)-6,8-difluoro-2-((tetrahydro-1H-pyrrolizin-7a(5H)-yl)methoxy)quinazolin-4-yl)-3-methylpiperidin-3-ol

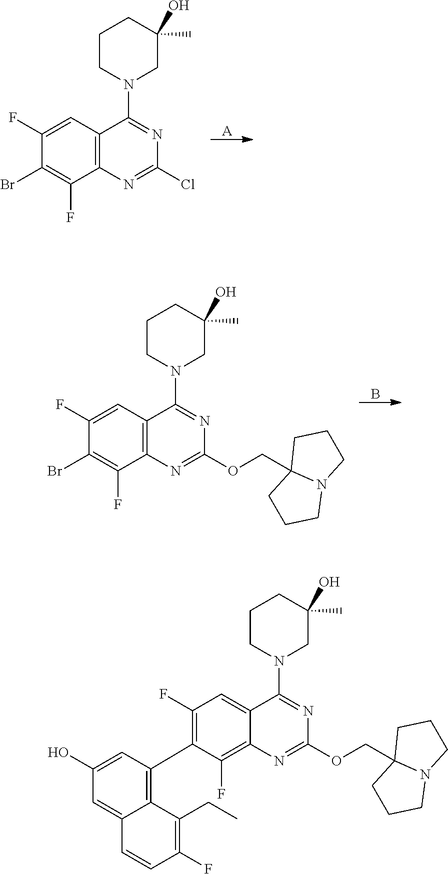

Step A. (R)-1-(7-bromo-6,8-difluoro-2-((hexahydro-1H-pyrrolizin-7a-yl)methoxy)quinazolin-4-yl)-3-methylpiperidin-3-ol: To a solution of (R)-1-(7-bromo-2-chloro-6,8-difluoroquinazolin-4-yl)-3-methylpiperidin-3-ol (200 mg, 1.0 equiv) in DMSO (0.3 mL) was added (hexahydro-1H-pyrrolizin-7a-yl)methanol (719 mg, 10 equiv). The mixture was stirred at 90° C. for 12 hours. The mixture was quenched with water (10 mL) and extracted with ethyl acetate (2×8 mL). The combined organic layers were dried over anhydrous sodium sulfate, concentrated and purified with reversed-phase flash (0.1% FA condition) to afford the title compound (130 mg, 42% yield) as white solid. LCMS (ESI, M+1, M+3): m/z=497.1, 499.1

Step B. (3R)-1-(7-(8-ethyl-7-fluoro-3-hydroxynaphthalen-1-yl)-6,8-difluoro-2-((hexahydro-1H-pyrrolizin-7a-yl)methoxy)quinazolin-4-yl)-3-methylpiperidin-3-ol: A mixture of (R)-1-(7-bromo-6,8-difluoro-2-((hexahydro-1H-pyrrolizin-7a-yl)methoxy)quinazolin-4-yl)-3-methylpiperidin-3-ol (100 mg, 1.0 equiv), 5-ethyl-6-fluoro-4-(4,4,5,5-tetramethyl-1,3,2-dioxaborolan-2-yl)naphthalen-2-ol (95.4 mg, 1.5 eq), Ad2nBuP-Pd-G3 (14.6 mg, 0.1 equiv), K₃PO₄ (2 M, 301 μL, 3.0 equiv) in CPME (3 mL) was degassed and purged with N₂ for 3 times, and then the mixture was stirred at 90° C. for 3 hours under N₂ atmosphere. The mixture was quenched with water (10 mL) and extracted with ethyl acetate (2×8 mL). The combined organic layers were dried over anhydrous sodium sulfate, concentrated and purified with reversed-phase flash (0.1% FA condition) to afford the title compound (130 mg, 42% yield) as white solid. SFC: Chiralpak IG-3 50×4.6 mm I.D., 3 m [40% EtOH (0.05% DEA)] in CO₂, flow rate: 3 mL/min, detector: 220 nm, t_{R1}=1.106 min, t_{R2}=1.615 min; ¹H NMR (400 MHz, METHANOL-d₄) δ=7.82 (br d, J=10.4 Hz, 1H), 7.68 (dd, J=5.6, 8.4 Hz, 1H), 7.34-7.18 (m, 2H), 6.98 (d, J=2.8 Hz, 1H), 4.47 (br s, 2H), 4.31 (br s, 1H), 4.11 (br d, J=13.2 Hz, 1H), 3.60-3.46 (m, 1H), 3.44-3.36 (m, 3H), 3.10-2.90 (m, 2H), 2.66-2.36 (m, 2H), 2.32-2.16 (m, 3H), 2.02 (br s, 4H), 1.96-1.92 (m, 2H), 1.86-1.82 (m, 1H), 1.82-1.72 (m, 2H), 1.36-1.22 (m, 3H), 0.81 (q, J=7.2 Hz, 3H); LCMS (ESI, M+1): m/z=607.2.

### Example 49

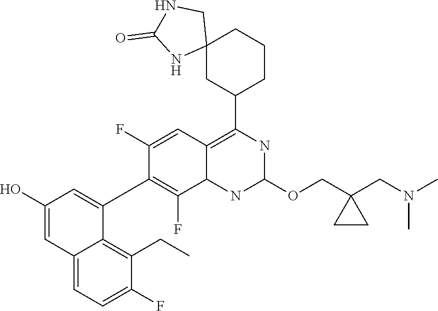

### 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,3,7-triazaspiro[4.5]decan-2-one

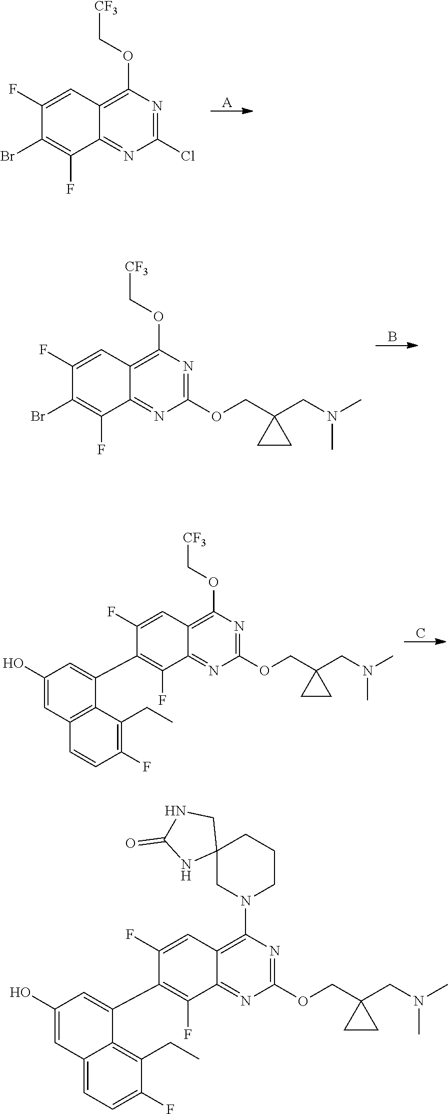

Step A. 1-(1-(((7-bromo-6,8-difluoro-4-(2,2,2-trifluoroethoxy)quinazolin-2-yl)oxy)methyl)cyclopropyl)-N,N-dimethylmethanamine: To a solution of 7-bromo-2-chloro-6,8-difluoro-4-(2,2,2-trifluoroethoxy)quinazoline (3.0 g, 1.0 equiv) and (1-((dimethylamino)methyl)cyclopropyl)methanol (1.03 g, 1.0 equiv) in DMF (15 mL) was added DIPEA (2.05 g, 2.0 equiv) and 4 Å molecular sieve (200 mg). The mixture was stirred at 40° C. for 14 hours under N₂ atmosphere. The reaction mixture was filtered. The filtrate was purified with reversed phase flash [water (FA, 0.1%)/acetonitrile=3/1] to afford the title compound (0.93 g, 22% yield) as light yellow solid; LCMS (ESI, M+1, M+3): m/z=470.1, 472.1.

Step B. 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol: To a mixture of 1-(1-(((7-bromo-6,8-difluoro-4-(2,2,2-trifluoroethoxy)quinazolin-2-yl)oxy)methyl)cyclopropyl)-N,N-dimethylmethanamine (1 g, 1 equiv) and 5-ethyl-6-fluoro-4-(4,4,5-trimethyl-1,3,2-dioxaborolan-2-yl)naphthalen-2-ol (1 g, 1.5 equiv) in methoxycyclopentane (20 mL) was added Cs₂CO₃ (1.5 M in H₂O, 4.25 mL, 3.0 equiv) and Ad₂nBuP-Pd-G3 (155 mg, 0.1 equiv) under N₂ atmosphere. The mixture was stirred at 70° C. for 12 hours. The mixture was diluted with H₂O (30 mL) and extracted with ethyl acetate (4×20 mL). The combined organic layer was dried over anhydrous Na₂SO₄, concentrated and purified with reversed phase flash [water (FA, 0.1%)/acetonitrile=3/2] to afford the tittle compound (0.87 g, 68% yield) was obtained as light yellow solid; LCMS (ESI, M+1): m/z=580.2.

Step C. 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,3,7-triazaspiro[4.5]decan-2-one: To a solution of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (170 mg, 1 equiv) and 1,3,7-triazaspiro[4.5]decan-2-one (91 mg, 2.0 equiv) in DMF (1 mL) was added 4 Å molecular sieve (20 mg) and K₃PO₄ (124 mg, 2.0 equiv). The mixture was stirred at 60° C. for 13 hours under N₂ atmosphere. The mixture was filtered. The filtrate was purified with prep-HPLC [column: Phenomenex Synergi C18 150×25 mm×10 μm; mobile phase: water(FA)−ACN; B %: 17%-47%, 10 minutes] to afford the tittle compound (17.3 mg, 9% yield, 0.27 FA) as white solid; ¹H NMR (400 MHz, methanol-d₄) δ=7.70-7.65 (m, 2H), 7.29 (d, J=2.4 Hz, 1H), 7.25 (t, J=9.2 Hz, 1H), 6.98 (d, J=2.8 Hz, 1H), 4.40-4.37 (m, 2H), 3.87-3.82 (m, 4H), 3.42 (d, J=9.2 Hz, 1H), 3.30-3.28 (m, 1H), 2.70-2.66 (m, 2H), 2.59-2.41 (m, 8H), 2.03-1.93 (m, 4H), 0.84-0.78 (m, 5H), 0.62-0.59 (m, 2H); LCMS (ESI, M+1): m/z=635.3.

### Example 50

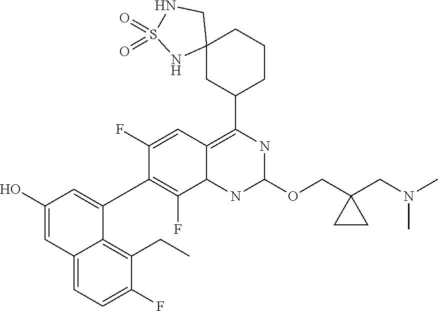

### 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-2-thia-1,3,7-triazaspiro[4.5]decane 2,2-dioxide

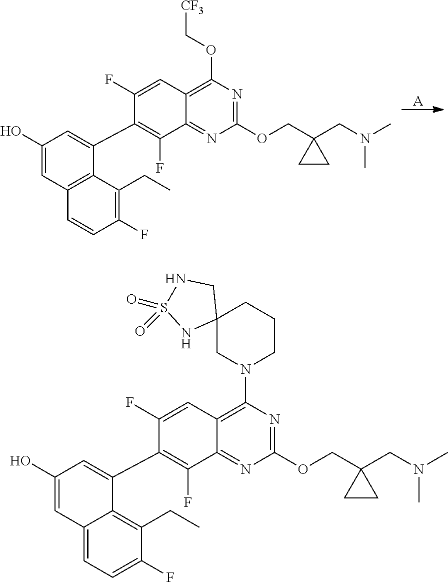

Step A. 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-2-thia-1,3,7-triazaspiro[4.5]decane 2,2-dioxide: To a solution of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (130 mg, 1 equiv) and 2-thia-1,3,7-triazaspiro[4.5]decane 2,2-dioxide (80 mg, 1.9 equiv) in DMF (0.9 mL) was added 4 Å molecular sieve (20 mg) and K₃PO₄ (95 mg, 2.0 equiv). The mixture was stirred at 60° C. for 16 hours under N₂ atmosphere. The mixture was filtered. The filtrate was purified with prep-HPLC [column: Phenomenex luna C18 150×25 mm×10 μm; mobile phase: water(FA)−ACN; B %: 20%-50%, 10 minutes] to afford the tittle compound (82.4 mg, 49% yield, 0.83 FA) as white solid; ¹H NMR (400 MHz, methanol-d4) δ=7.73-7.67 (m, 2H), 7.30 (d, J=2.8 Hz, 1H), 7.28-7.22 (m, 1H), 6.99 (dd, J=2.8, 6.8 Hz, 1H), 4.47-4.31 (m, 3H), 4.25-4.15 (br s, 1H), 3.69 (br t, J=13.2 Hz, 1H), 3.63-3.53 (m, 1H), 3.40 (dd, J=3.2, 11.8 Hz, 1H), 3.21 (d, J=12.0 Hz, 1H), 3.15 (br d, J=3.2 Hz, 2H), 2.87 (s, 6H), 2.65-2.51 (m, 1H), 2.43 (ddd, J=3.6, 7.2, 10.4 Hz, 1H), 2.04 (br d, J=11.2 Hz, 2H), 1.93-1.87 (m, 2H), 0.95-0.91 (m, 2H), 0.81-0.79 (m, 5H); LCMS (ESI, M+1): m/z=671.3.

### Example 51

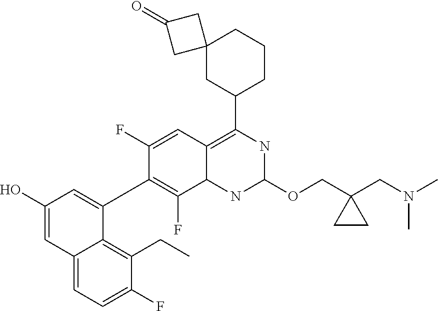

### 6-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,6-diazaspiro[3.5]nonan-2-one

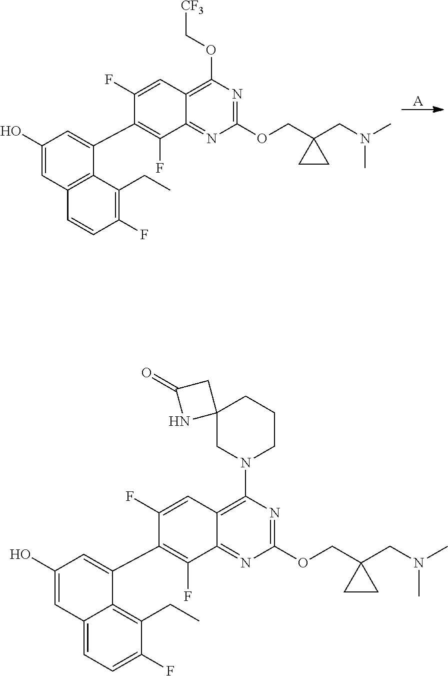

Step A. 6-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,6-diazaspiro[3.5]nonan-2-one: To a solution of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (120 mg, 1 equiv) and 1,6-diazaspiro[3.5]nonan-2-one (60 mg, 2.1 equiv) in DMF (0.9 mL) was added 4 Å molecular sieve (20 mg) and K₃PO₄ (88 mg, 2.0 equiv). The mixture was stirred at 60° C. for 11 hours under N₂ atmosphere. The mixture was filtered. The filtrate was purified with prep-HPLC [column: Phenomenex Synergi C18 150×25 mm×10 μm; mobile phase: water(FA)−ACN; B %: 17%-47%, 10 minutes] to afford the tittle compound (70.0 mg, 50% yield, 0.67 FA) as white solid; ¹H NMR (400 MHz, methanol-d4) δ=7.70-7.65 (m, 2H), 7.30 (d, J=2.8 Hz, 1H), 7.25 (t, J=9.2 Hz, 1H), 6.99 (d, J=2.4 Hz, 1H), 4.41-4.39 (m, 2H), 4.14-4.11 (m, 2H), 3.87-3.84 (m, 1H), 3.66 (ddd, J=4.0, 8.4, 12.4 Hz, 1H), 3.34-2.98 (m, 2H), 2.89-2.83 (m, 1H), 2.77 (s, 6H), 2.75-2.73 (m, 1H), 2.62-2.49 (m, 1H), 2.42 (ddd, J=2.8, 7.2, 14.4 Hz, 1H), 2.05-1.95 (m, 4H), 0.91-0.88 (m, 2H), 0.80 (t, J=7.3 Hz, 3H), 0.77-0.72 (m, 2H); LCMS (ESI, M+1): m/z=620.3.

### Example 52

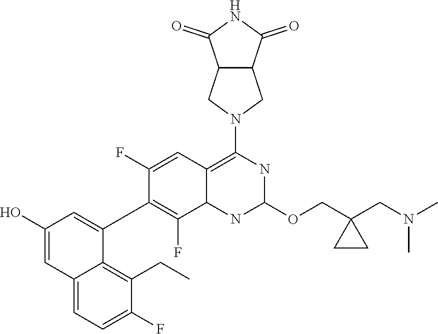

### 5-(2-((1-((dimethylamino)methyl)cyclopropyl)methoxy)-7-(8-ethly-7-fluoro-3-hydroxynaphthalen-1-yl)-6,8-difluoroquinazolin-4-yl)tetrahydropyrrolo[3,4-c]pyrrole-1,3(2H,3aH)-dione

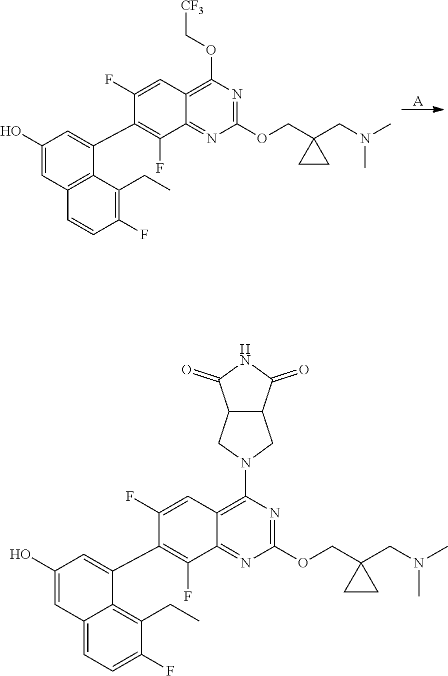

Step A. 5-(2-((1-((dimethylamino)methyl)cyclopropyl)methoxy)-7-(8-ethyl-7-fluoro-3-hydroxynaphthalen-1-yl)-6,8-difluoroquinazolin-4-yl)tetrahydropyrrolo[3,4-c]pyrrole-1,3(2H,3aH)-dione: To a solution of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (130 mg, 1 equiv) and tetrahydropyrrolo[3,4-c]pyrrole-1,3(2H,3aH)-dione (65 mg, 2.1 equiv) in DMF (0.9 mL) was added 4 Å molecular sieve (20 mg) and K₃PO₄ (95 mg, 2.0 equiv). The mixture was stirred at 60° C. for 13 hours under N₂ atmosphere. The mixture was filtered. The filtrate was purified with prep-HPLC [column: Phenomenex Synergi C18 150×25 mm×10 μm; mobile phase: water(FA)−ACN; B %: 18%-48%, 10 minutes] to afford the tittle compound (51.5 mg, 35% yield, 0.73 FA) as white solid; ¹H NMR (400 MHz, methanol-d₄) δ=7.87 (br d, J=10.0 Hz, 1H), 7.68 (dd, J=6.0, 8.8 Hz, 1H), 7.30 (d, J=2.4 Hz, 1H), 7.25 (t, J=9.2 Hz, 1H), 7.00 (d, J=2.4 Hz, 1H), 4.65 (br dd, J=5.2, 13.2 Hz, 2H), 4.41-4.38 (m, 2H), 4.18 (br dd, J=8.8, 12.0 Hz, 2H), 3.69 (br d, J=7.6 Hz, 2H), 3.10-3.02 (m, 2H), 2.81-2.78 (m, 6H), 2.55-2.49 (m, 1H), 2.47-2.42 (m, 1H), 0.91-0.88 (m, 2H), 0.81-0.76 (m, 5H); LCMS (ESI, M+1): m/z=620.3.

### Example 53

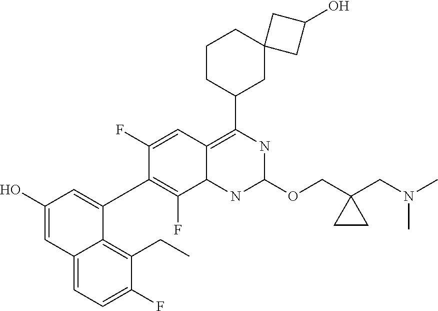

### 6-(2-((1-((dimethylamino)methyl)cyclopropyl)methoxy)-7-(8-ethyl-7-fluoro-3-hydroxynaphthalen-1-yl)-6,8-difluoroquinazolin-4-yl)-6-azaspiro[3.5]nonan-2-ol

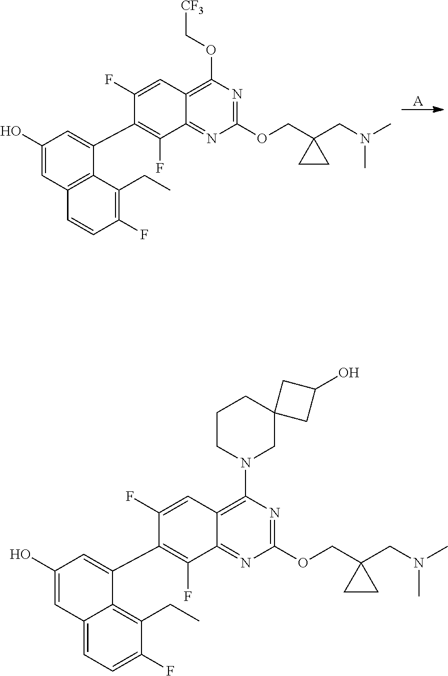

Step E. 6-(2-((1-((dimethylamino)methyl)cyclopropyl)methoxy)-7-(8-ethyl-7-fluoro-3-hydroxynaphthalen-1-yl)-6,8-difluoroquinazolin-4-yl)-6-azaspiro[3.5]nonan-2-ol: To a solution of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (130 mg, 1 equiv) and 6-azaspiro[3.5]nonan-2-ol (80 mg, 2.5 equiv) in DMF (0.9 mL) was added 4 Å molecular sieve (20 mg) and K₃PO₄ (95 mg, 2.0 equiv). The mixture was stirred at 60° C. for 16 hours under N₂ atmosphere. The mixture was filtered. The filtrate was purified with prep-HPLC [column: Phenomenex Synergi C18 150×25 mm×10 μm; mobile phase: water(FA)−ACN; B %: 18%-48%, 10 minutes] to afford the tittle compound (54.6 mg, 36% yield, 0.5 FA) as white solid; ¹H NMR (400 MHz, methanol-d₄) δ=7.67 (dd, J=6.0, 9.2 Hz, 1H), 7.65-7.62 (m, 1H), 7.30 (d, J=2.8 Hz, 1H), 7.24 (t, J=9.2 Hz, 1H), 7.00 (d, J=2.4 Hz, 1H), 4.39 (d, J=3.2 Hz, 2H), 4.30-4.26 (m, 1H), 3.91-3.83 (m, 2H), 3.80-3.77 (m, 2H), 2.93 (br d, J=4.8 Hz, 2H), 2.68 (br d, J=3.6 Hz, 6H), 2.56 (br s, 1H), 2.44 (dt, J=2.8, 7.2 Hz, 1H), 2.35-2.18 (m, 2H), 1.82-1.70 (m, 6H), 0.86-0.78 (m, 5H), 0.74-0.71 (m, 2H); LCMS (ESI, M+1): m/z=621.3.

### Example 54

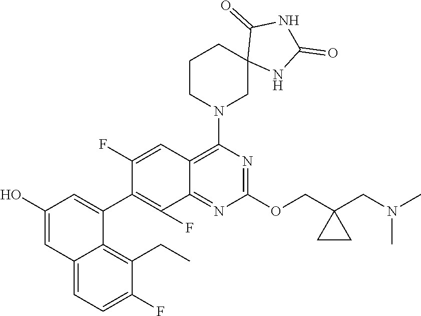 [SMILES: CCc1c(F)ccc2cc(O)cc(-c3c(F)cc4c(N5CCCC6(C5)NC(=O)NC6=O)nc(OCC5(CN(C)C)CC5)nc4c3F)c12]

### 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,3,7-triazaspiro[4.5]decane-2,4-dione

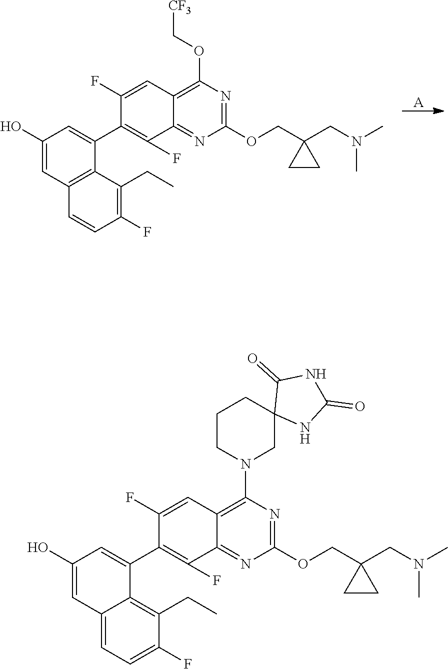

Step A. 7-(2-((1-((dimethylamino)methyl)cyclopropyl)methoxy)-7-(8-ethyl-7-fluoro-3-hydroxynaphthalen-1-yl)-6,8-difluoroquinazolin-4-yl)-1,3,7-triazaspiro[4.5]decane-2,4-dione: To a mixture of 4-(2-((1-((dimethylamino)methyl)cyclopropyl)methoxy)-6,8-difluoro-4-(2,2,2-trifluoroethoxy)quinazolin-7-yl)-5-ethyl-6-fluoronaphthalen-2-ol (60.0 mg, 1.0 equiv), 1,3,7-triazaspiro[4.5]decane-2,4-dione (35.0 mg, 2.0 equiv) and 4 Å molecular sieve (10.0 mg, 1.0 equiv) in MeCN (0.2 mL) and DMF (0.2 mL) was added K₃PO₄ (65.9 mg, 3.0 equiv). The mixture was stirred at 40° C. for 12 hrs. The reaction mixture was filtered and purified by prep-HPLC [column: Phenomenex Synergi C18 150×25 mm×10 um; mobile phase: [water(FA)−ACN]; B %: 18%-40%,11 min] and lyophilized to afford the title compound (19.8 mg, 29% yield) as white solid; H NMR (400 MHz, METHANOL-d₄) δ=7.73-7.66 (m, 2H), 7.32-7.30 (m, 1H), 7.26 (t, J=9.2 Hz, 1H), 7.01-6.96 (m, 1H), 4.45-4.36 (m, 3H), 4.35-4.34 (m, 1H), 4.35 (br s, 1H), 3.73-3.64 (m, 1H), 3.64-3.55 (m, 1H), 3.19-3.10 (m, 2H), 2.85 (s, 6H), 2.62-2.51 (m, 1H), 2.49-2.36 (m, 1H), 2.27-2.17 (m, 1H), 2.10-2.01 (m, 2H), 1.96 (br d, J=13.6 Hz, 1H), 0.93 (m, 2H), 0.83-0.76 (m, 5H); LCMS (ESI, M+1): m/z=649.4.

### Example 55

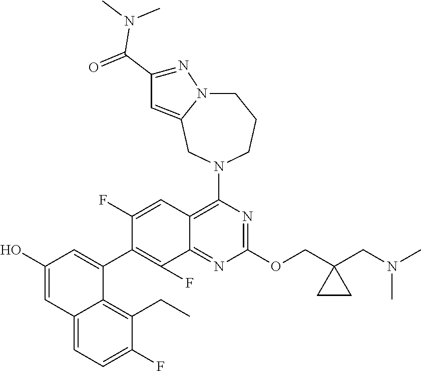 [SMILES: CCc1c(F)ccc2cc(O)cc(-c3c(F)cc4c(N5CCCn6nc(C(=O)N(C)C)cc6C5)nc(OCC5(CN(C)C)CC5)nc4c3F)c12]

### 5-(2-((1-((dimethylamino)methyl)cyclopropyl)methoxy)-7-(8-ethyl-7-fluoro-3-hydroxynaphthalen-1-yl)-6,8-difluoroquinazolin-4-yl)-N,N-dimethyl-5,6,7,8-tetrahydro-4H-pyrazolo[1,5-a][1,4]diazepine-2-carboxamide

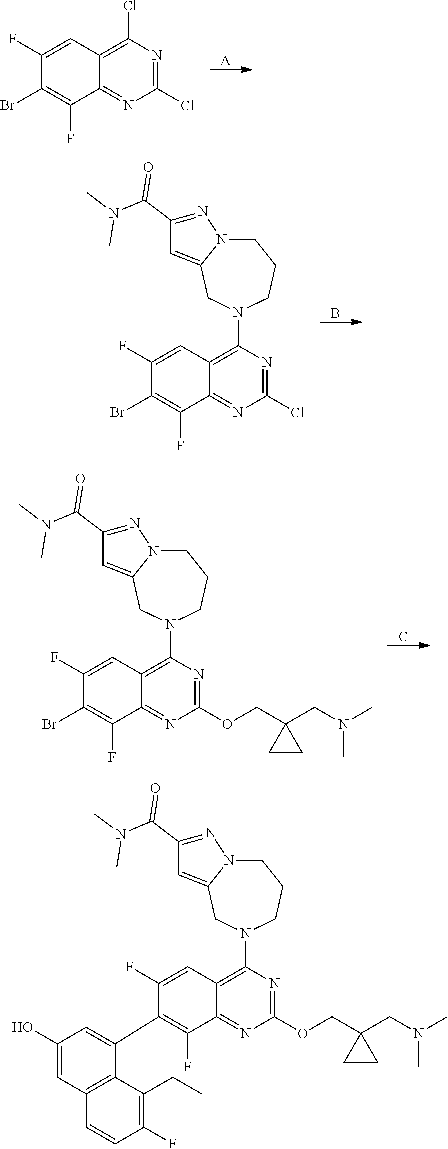

Step A. 5-(7-bromo-2-chloro-6,8-difluoroquinazolin-4-yl)-N,N-dimethyl-5,6,7,8-tetrahydro-4H-pyrazolo[1,5-a][1,4]diazepine-2-carboxamide: To a mixture of 7-bromo-2,4-dichloro-6,8-difluoroquinazoline (1.00 g, 1.0 equiv), N,N-dimethyl-5,6,7,8-tetrahydro-4H-pyrazolo[1,5-a][1,4]diazepine-2-carboxamide (597 mg, 0.9 equiv) and 4 Å molecular sieve (100 mg, 1.0 equiv) in DCM (10 mL) was added DIEA (1.24 g, 3 equiv) at 0° C., and then the mixture was stirred at 20° C. for 2 hrs. The reaction mixture was filtered and purified by reversed phase flash [C18, 0.1% formic acid condition] to afford the title compound (1.50 g, 89% yield) as white solid; LCMS (ESI, M+1, M+3): m/z=485.0, 487.0.

Step B. 5-(7-bromo-2-((1-((dimethylamino)methyl)cyclopropyl)methoxy)-6,8-difluoroquinazolin-4-yl)-N,N-dimethyl-5,6,7,8-tetrahydro-4H-pyrazolo[1,5-a][1,4]diazepine-2-carboxamide: To a mixture of 5-(7-bromo-2-chloro-6,8-difluoroquinazolin-4-yl)-N,N-dimethyl-5,6,7,8-tetrahydro-4H-pyrazolo[1,5-a][1,4]diazepine-2-carboxamide (500 mg, 1.0 equiv), (1-((dimethylamino)methyl)cyclopropyl)methanol (399 mg, 3.0 equiv), 4 Å molecular sieve (100 mg, 1.0 equiv) in DMF (5 mL) was added DIEA (399 mg, 3.0 equiv), and then the mixture was stirred at 110° C. for 36 hrs. The reaction mixture was filtered and purified by reversed phase flash [C18, 0.1% formic acid condition] to afford the title compound (200 mg, 28% yield) as white solid; LCMS (ESI, M+1, M+3): m/z=578.1, 580.1.

Step C. 5-(2-((1-((dimethylamino)methyl)cyclopropyl)methoxy)-7-(8-ethyl-7-fluoro-3-hydroxynaphthalen-1-yl)-6,8-difluoroquinazolin-4-yl)-N,N-dimethyl-5,6,7,8-tetrahydro-4H-pyrazolo[1,5-a][1,4]diazepine-2-carboxamide: To a mixture of 5-(7-bromo-2-((1-((dimethylamino)methyl)cyclopropyl)methoxy)-6,8-difluoroquinazolin-4-yl)-N,N-dimethyl-5,6,7,8-tetrahydro-4H-pyrazolo[1,5-a][1,4]diazepine-2-carboxamide (180 mg, 1.0 equiv), 5-ethyl-6-fluoro-4-(4,4,5,5-tetramethyl-1,3,2-dioxaborolan-2-yl)naphthalen-2-ol (197 mg, 2.0 equiv), Cs₂CO₃ (304 mg, 1.5 M in water, 3 equiv) and [2-(2-aminophenyl)phenyl]palladium(1+);bis(1-adamantyl)-butyl-phosphane;methanesulfonate (22.7 mg, 0.1 equiv) in CPME (5 mL) was degassed and purged with N₂ for 3 times, and then the mixture was stirred a 60° C. for 12 hours under N₂ atmosphere. The mixture was diluted with water (10 mL), filtered and extracted with EtOAc (3×5 mL). The combined organic layers were washed with brine (5 mL), dried over anhydrous sodium sulfate, concentrated and purified by reversed phase flash [C18, 0.1% formic acid condition] to afford the title compound (115 mg, 51% yield) as yellow solid; ¹H NMR (400 MHz, METHANOL-d₄) δ=7.73-7.65 (m, 2H), 7.30-7.28 (m, 1H), 7.27-7.21 (m, 1H), 7.00-6.96 (m, 1H), 6.69-6.65 (m, 1H), 5.20-5.05 (m, 2H), 4.57-4.52 (m, 2H), 4.36-4.24 (m, 4H), 3.35 (m, 3H), 3.09-3.06 (m, 3H), 2.61-2.53 (m, 1H), 2.46 (br d, J=6.4 Hz, 2H), 2.43-2.36 (m, 3H), 2.33-2.30 (m, 6H), 0.82-0.77 (m, 3H), 0.73-0.69 (m, 2H), 0.54-0.49 (m, 2H); LCMS (ESI, M+1): m/z=688.4.

### Example 56

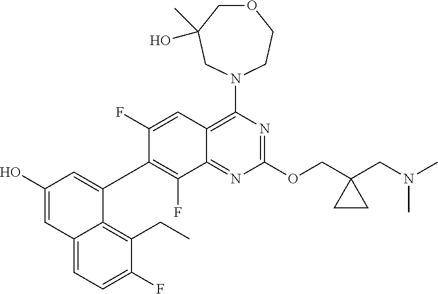 [SMILES: CCc1c(F)ccc2cc(O)cc(-c3c(F)cc4c(N5CCOCC(C)(O)C5)nc(OCC5(CN(C)C)CC5)nc4c3F)c12]

### 4-(2-((1-((dimethylamino)methyl)cyclopropyl)methoxy)-7-(8-ethyl-7-fluoro-3-hydroxynaphthalen-1-yl)-6,8-difluoroquinazolin-4-yl)-6-methyl-1,4-oxazepan-6-ol

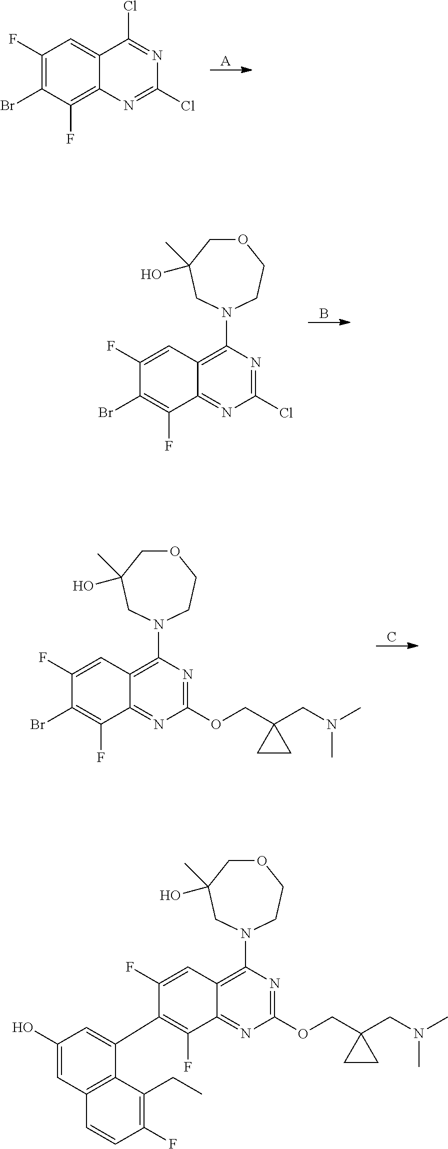

Step A. 4-(7-bromo-2-chloro-6,8-difluoroquinazolin-4-yl)-6-methyl-1,4-oxazepan-6-ol: To a solution of 7-bromo-2,4-dichloro-6,8-difluoro-quinazoline (0.5 g, 1.0 equiv) and DIPEA (411 mg, 2.0 equiv) in DCM (10 mL) was added 6-methyl-1,4-oxazepan-6-ol (198 mg, 0.95 equiv) in DCM (5 mL) dropwise at 0° C. The mixture was stirred at 15° C. for 1 hours. The mixture was diluted with H₂O (20 mL) at 0° C. and extracted with DCM (3×10 mL). The combined organic layers were dried over anhydrous Na₂SO₄, concentrated and purified with column chromatography [SiO₂, petroleum ether/ethyl acetate=2/1 to 1/1] to afford the title compound (0.5 g, 76% yield) as light yellow solid; LCMS (ESI, M+1, M+3): m/z=408.0, 410.0.

Step B. 4-(7-bromo-2-((1-((dimethylamino)methyl)cyclopropyl)methoxy)-6,8-difluoroquinazolin-4-yl)-6-methyl-1,4-oxazepan-6-ol: To a mixture of 4-(7-bromo-2-chloro-6,8-difluoroquinazolin-4-yl)-6-methyl-1,4-oxazepan-6-ol (0.5 g, 1.0 equiv) and (1-((dimethylamino)methyl)cyclopropyl)methanol (316 mg, 2.0 equiv) in DMSO (2.5 mL) was added DIPEA (319 mg, 2.0 equiv) and 4 Å molecular sieve (30 mg). The mixture was stirred at 90° C. for 13 hours. The reaction mixture was filtered. The filtrate was purified with reversed phase flash [water (FA, 0.1%)/acetonitrile=3/2] to afford the title compound (0.35 g, 50% yield) as light-yellow solid; LCMS (ESI, M+1, M+3): m/z=501.1, 503.1.

Step C. 4-(2-((1-((dimethylamino)methyl)cyclopropyl)methoxy)-7-(8-ethyl-7-fluoro-3-hydroxynaphthalen-1-yl)-6,8-difluoroquinazolin-4-yl)-6-methyl-1,4-oxazepan-6-ol: To a solution of 4-(7-bromo-2-((1-((dimethylamino)methyl)cyclopropyl)methoxy)-6,8-difluoroquinazolin-4-yl)-6-methyl-1,4-oxazepan-6-ol (0.35 g, 1.0 equiv) and 5-ethyl-6-fluoro-4-(4,4,5,5-tetramethyl-1,3,2-dioxaborolan-2-yl)naphthalen-2-ol (255 mg, 1.3 equiv) in methoxycyclopentane (6.25 mL) was K₃PO₄ (1.5 M in H₂O, 1.23 mL, 3.0 equiv) and Ad₂nBuP-Pd-G3 (45 mg, 0.1 equiv) under N₂ atmosphere. The mixture was stirred at 80° C. for 3 hours. The mixture was diluted with H₂O (2 mL) and extracted with ethyl acetate (4×3 mL). The combined organic layer was dried over anhydrous Na₂SO₄ and concentrated. The residue was purified with reversed phase flash [water (FA, 0.1%)/acetonitrile=13/7] and prep-HPLC [column: Waters Xbridge 150×25 mm×5 μm; mobile phase: water (NH₄HCO₃)−ACN; B %: 59%-89%, 10 minutes] to afford the tittle compound (156 mg, 40% yield, 0.26 FA) as white solid; ¹H NMR (400 MHz, methanol-d4) δ=8.16-8.09 (m, 1H), 7.68 (dd, J=6.0, 9.2 Hz, 1H), 7.30 (d, J=2.8 Hz, 1H), 7.25 (t, J=9.2 Hz, 1H), 7.01-6.96 (m, 1H), 4.46-4.42 (m, 1H), 4.41-4.35 (m, 3H), 4.04-3.97 (m, 2H), 3.93-3.84 (m, 2H), 3.38-3.64 (m, 2H), 2.69-2.54 (m, 3H), 2.49 (s, 6H), 2.45-2.36 (m, 1H), 1.27-1.25 (m, 3H), 0.85-0.77 (m, 5H), 0.63-0.58 (s, 2H); LCMS (ESI, M+1): m/z=611.4.

### Example 57

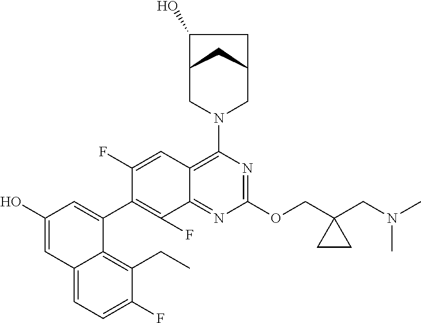 [SMILES: CCc1c(F)ccc2cc(O)cc(-c3c(F)cc4c(N5C[C@@H]6C[C@H](C5)[C@H](O)C6)nc(OCC5(CN(C)C)CC5)nc4c3F)c12]

### (1R,5R,6R)-3-(2-((1-((dimethylamino)methyl)cyclopropyl)methoxy)-7-(8-ethyl-7-fluoro-3-hydroxynaphthalen-1-yl)-6,8-difluoroquinazolin-4-yl)-3-azabicyclo[3.2.1]octan-6-ol

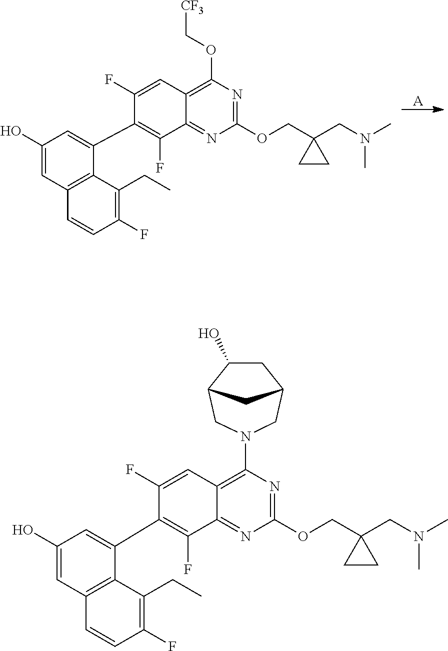

Step A. (1R,5R,6R)-3-(2-((1-((dimethylamino)methyl)cyclopropyl)methoxy)-7-(8-ethyl-7-fluoro-3-hydroxynaphthalen-1-yl)-6,8-difluoroquinazolin-4-yl)-3-azabicyclo[3.2.1]octan-6-ol: To a mixture of 4-[2-[[1-[(dimethylamino)methyl]cyclopropyl]methoxy]-6,8-difluoro-4-(2,2,2-trifluoroethoxy)quinazolin-7-yl]-5-ethyl-6-fluoro-naphthalen-2-ol (117 mg, 1.0 equiv), (1R,5R,6R)-3-azabicyclo[3.2.1]octan-6-ol (51.35 mg, 2.0 equiv) in MeCN (5 mL) and DMF (5 mL) was added K₃PO₄ (128 mg, 3.0 equiv). The mixture was stirred at 40° C. for 12 hrs. The mixture was filtered and purified by prep-HPLC (column: Phenomenex luna C18 150×25 mm×10 um; mobile phase: [water (FA)−ACN]; B %: 36%-56%, 2 min) to afford the title compound (27 mg, 22% yield) as white solid; ¹H NMR (400 MHz, CHLOROFORM-d) δ=7.59-7.52 (m, 1H), 7.43 (br d, J=10.0 Hz, 1H), 7.27-7.24 (m, 1H), 7.21-7.13 (m, 1H), 7.10-6.94 (m, 1H), 4.91 (br d, J=11.6 Hz, 1H), 4.75-4.71 (m, 1H), 4.53-4.37 (m, 1H), 4.33 (br s, 2H), 4.21-3.95 (m, 1H), 3.76-3.54 (m, 1H), 3.37-3.28 (m, 1H), 2.99-2.90 (m, 1H), 2.89-2.72 (m, 1H), 2.70-2.59 (m, 6H), 2.58-2.33 (m, 2H), 2.33-2.23 (m, 2H), 2.21-2.04 (m, 1H), 1.83-1.70 (m, 2H), 1.35-1.01 (m, 1H), 0.87-0.75 (m, 4H), 0.71-0.62 (m, 2H); LCMS (ESI, M+1): m/z=607.3.

### Example 58

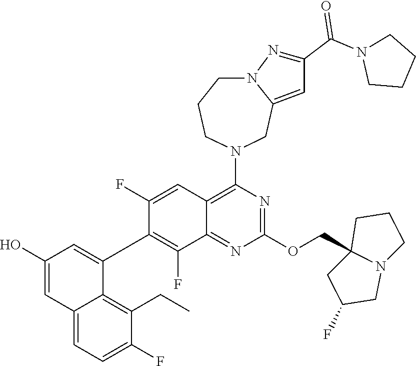 [SMILES: CCc1c(F)ccc2cc(O)cc(-c3c(F)cc4c(N5CCCn6nc(C(=O)N7CCCC7)cc6C5)nc(OC[C@@]56CCCN5C[C@H](F)C6)nc4c3F)c12]

### (5-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-5,6,7,8-tetrahydro-4H-pyrazolo[1,5-a][1,4]diazepin-2-yl)(pyrrolidin-1-yl)methanone

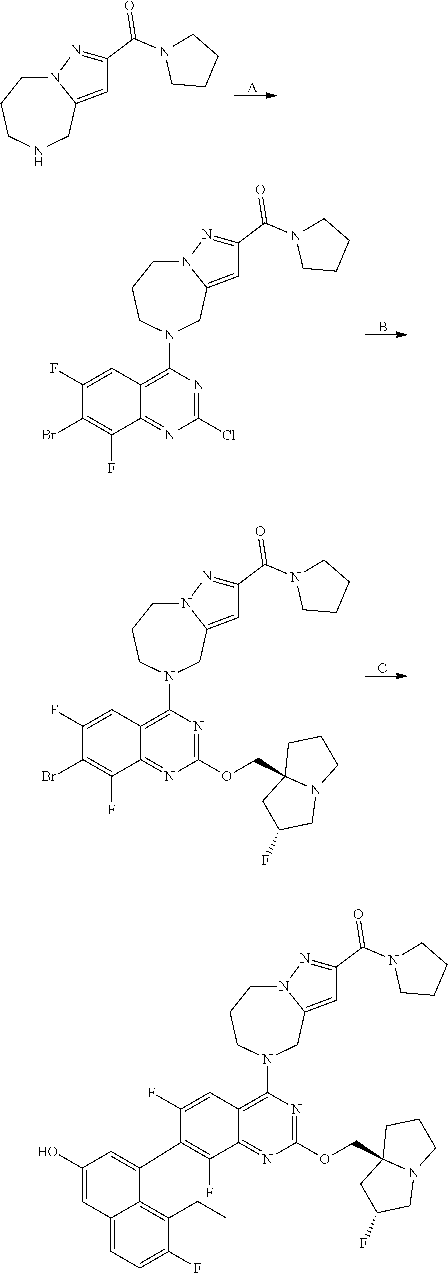

Step A. (5-(7-bromo-2-chloro-6,8-difluoroquinazolin-4-yl)-5,6,7,8-tetrahydro-4H-pyrazolo[1,5-a][1,4]diazepin-2-yl)(pyrrolidin-1-yl)methanone: To a solution of 7-bromo-2,4-dichloro-6,8-difluoroquinazoline (500 mg, 1.0 equiv) in DCM (4 mL) were added DIEA (1.65 g, 8.0 equiv) and pyrrolidin-1-yl(5,6,7,8-tetrahydro-4H-pyrazolo[1,5-a][1,4]diazepin-2-yl)methanone (560 mg, 1.5 equiv). The mixture was stirred at 0° C. for 0.5 hour. The reaction mixture was concentrated under reduced pressure to remove solvent and purified by column chromatography (SiO₂, petroleum ether/ethyl acetate=5/1 to dichloromethane: methanol=10:1) to afford the title compound (460 mg, 56% yield) as yellow solid; LCMS (ESI, M+1, M+3): m/z=510.8, 512.8.

Step B. (5-(7-bromo-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(pyrrolidin-1-yl)methanone: To a solution of (5-(7-bromo-2-chloro-6,8-difluoroquinazolin-4-yl)-5,6,7,8-tetrahydro-4H-pyrazolo[1,5-a][1,4]diazepin-2-yl)(pyrrolidin-1-yl)methanone (240 mg, 1.0 equiv) in DMAc (5 mL) was added CsF (712 mg, 10 equiv) and ((2R,7aS)-2-fluorohexahydro-1H-pyrrolizin-7a-yl)methanol (373 mg, 5.0 equiv). The mixture was stirred at 60° C. for 12 hours. The reaction mixture was filtered and purified by prep-HPLC [column: YMC Triart C18 250×50 mm×7 μm; mobile phase: (water (NH₃H₂O)−ACN); B %: 47%-77% over 20 min] to afford the title compound (70 mg, 23% yield) as white solid; LCMS (ESI, M+1, M+3): m/z=634.3, 636.3.

Step C. (5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(pyrrolidin-1-yl)methanone: To a solution of (5-(7-bromo-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(pyrrolidin-1-yl)methanone (60 mg, 1.0 equiv) and 5-ethyl-6-fluoro-4-(4,4,5,5-tetramethyl-1,3,2-dioxaborolan-2-yl)naphthalen-2-ol (89.7 mg, 3.0 equiv) in methoxycyclopentane (1 mL) were added K₃PO₄ (60.2 mg, 3 equiv) and Ad₂nBuP-Pd-G3 (6.89 mg, 0.1 equiv). The mixture was degassed and purged with N₂ for 3 times and stirred at 90° C. for 1.5 hours under N₂ atmosphere. The reaction mixture was filtered and purified by prep-HPLC [column: Phenomenex luna C18 150×25 mm×10 μm; A: water (10 mM HCOOH), B: ACN, B %: 24%-54% over 10 min] to afford the title compound (4.17 mg, 5.9% yield) as white solid; ¹H NMR (400 MHz, METHANOL-d₄) δ=7.80-7.64 (m, 2H), 7.33-7.20 (m, 2H), 6.98 (d, J=2.4 Hz, 1H), 6.79 (s, 1H), 5.46-5.26 (m, 1H), 5.21-5.06 (m, 2H), 4.58-4.51 (m, 2H), 4.38-4.24 (m, 4H), 3.93 (t, J=6.4 Hz, 2H), 3.59 (t, J=6.8 Hz, 2H), 3.43-3.36 (m, 2H), 3.19-3.06 (m, 2H), 2.64-2.52 (m, 1H), 2.48-2.35 (m, 4H), 2.34-2.26 (m, 1H), 2.24-2.16 (m, 1H), 2.12-2.03 (m, 2H), 2.00-1.90 (m, 5H), 0.80 (br t, J=7.2 Hz, 3H); LCMS (ESI, M+1): m/z=744.4

### Example 59

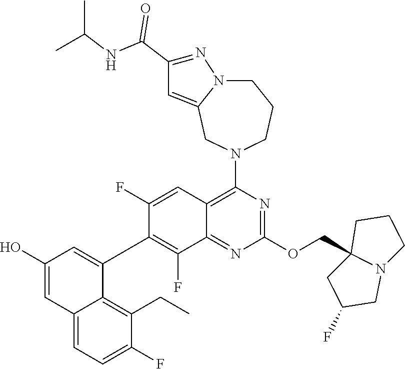 [SMILES: CCc1c(F)ccc2cc(O)cc(-c3c(F)cc4c(N5CCCn6nc(C(=O)NC(C)C)cc6C5)nc(OC[C@@]56CCCN5C[C@H](F)C6)nc4c3F)c12]

### 5-(7-(8-ethyl-7-fluoro-3-hydroxynaphthalen-1-yl)-6,8-difluoro-2-(((2R,7aS)-2-fluorotetrahydro-1H-pyrrolizin-7a(51)-yl)methoxy)quinazolin-4-yl)-N-isopropyl-5,6,7,8-tetrahydro-4H-pyrazolo[1,5-a][1,4]diazepine-2-carboxamide

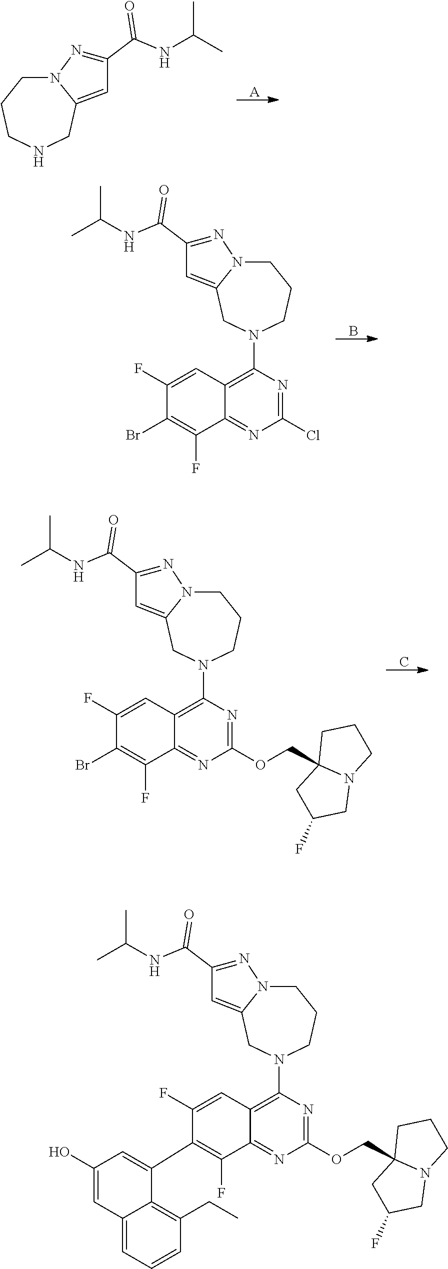

Step A. 5-(7-bromo-2-chloro-6,8-difluoroquinazolin-4-yl)-N-isopropyl-5,6,7,8-tetrahydro-4H-pyrazolo[1,5-a][1,4]diazepine-2-carboxamide: To a solution of 7-bromo-2,4-dichloro-6,8-difluoroquinazoline (300 mg, 1.0 equiv), 4 Å molecular sieve (10 mg) in DCM (0.5 mL) was added DIEA (370 mg, 3.0 equiv) and N-isopropyl-5,6,7,8-tetrahydro-4H-pyrazolo[1,5-a][1,4]diazepine-2-carboxamide (297 mg, 1.2 equiv, HCl). The mixture was stirred at 0° C. for 1 hour. The mixture was concentrated under vacuum to give a residue. The residue was purified by reversed phase flash [C18, 0.1% formic acid condition] to afford the title compound (201 mg, 38% yield) as yellow solid. LCMS (ESI, M+1, M+3): m/z=498.8, 500.8.

Step B. 5-(7-bromo-6,8-difluoro-2-(((2R,7aS)-2-fluorohexahydro-1H-pyrrolizin-7a-yl)methoxy)quinazolin-4-yl)-N-isopropyl-5,6,7,8-tetrahydro-4H-pyrazolo[1,5-a][1,4]diazepine-2-carboxamide: To a solution of 5-(7-bromo-2-chloro-6,8-difluoroquinazolin-4-yl)-N-isopropyl-5,6,7,8-tetrahydro-4H-pyrazolo[1,5-a][1,4]diazepine-2-carboxamide (180 mg, 1.0 equiv) and 4 Å molecular sieve (10 mg) in dioxane (1 mL) was added DIEA (186 mg, 4.0 equiv) and ((2R,7aS)-2-fluorohexahydro-1H-pyrrolizin-7a-yl)methanol (287 mg, 5.0 equiv). The mixture was stirred at 90° C. for 5 hours. The mixture was filtered to give a residue. The residue was purified by reversed phase flash [C18, 0.1% formic acid condition] to afford the title compound (60 mg, 26% yield) as yellow solid. LCMS (ESI, M+1, M+3): m/z=621.8, 623.8.

Step C. 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N-isopropyl-5,6,7,8-tetrahydro-4H-pyrazolo[1,5-a][1,4]diazepine-2-carboxamide: To a mixture of 5-(7-bromo-6,8-difluoro-2-(((2R,7aS)-2-fluorohexahydro-1H-pyrrolizin-7a-yl)methoxy)quinazolin-4-yl)-N-isopropyl-5,6,7,8-tetrahydro-4H-pyrazolo[1,5-a][1,4]diazepine-2-carboxamide (45.0 mg, 1.0 equiv), 5-ethyl-6-fluoro-4-(4,4,5,5-tetramethyl-1,3,2-dioxaborolan-2-yl)naphthalen-2-ol (68.6 mg, 3.0 equiv) and K₃PO₄ (46.0 mg, 3.0 equiv) in methoxy-cyclopentane (0.5 mL) was added Ad₂nBuP-Pd-G3 (5.30 mg, 0.1 equiv). The mixture was degassed and purged with N₂ for 3 times. The mixture was stirred at 90° C. for 1.5 hours under N₂ atmosphere. The mixture was filtered to give a residue. The residue was purified by prep-HPLC (column: Phenomenex luna C18 150×25 mm×10 μm; mobile phase: [water (FA)−ACN]; B %: 20%-50%, 10 min) to afford the title compound (9.4 mg, 17% yield, FA) as white solid. ¹H NMR (400 MHz, METHANOL-d₄) δ=7.77 (d, J=10.0 Hz, 1H), 7.68 (dd, J=6.0, 9.2 Hz, 1H), 7.30-7.23 (m, 2H), 6.98 (d, J=2.4 Hz, 1H), 6.77 (s, 1H), 5.48-5.34 (m, 1H), 5.20-5.04 (m, 2H), 4.52-4.49 (m, 1H), 4.40-4.36 (m, 2H), 4.34-4.28 (m, 2H), 4.16 (td, J=6.8, 13.2 Hz, 1H), 3.52-3.46 (m, 2H), 3.22-3.13 (m, 2H), 2.61-2.50 (m, 1H), 2.47-2.30 (m, 5H), 2.29-2.10 (m, 4H), 2.02-1.94 (m, 1H), 1.23 (d, J=6.8 Hz, 6H), 0.80 (t, J=7.6 Hz, 3H). LCMS (ESI, M+1): m/z=732.5.

### Example 60

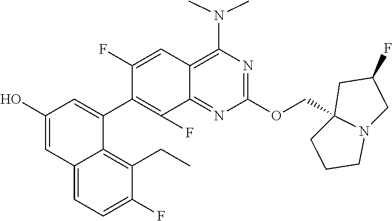 [SMILES: CCc1c(F)ccc2cc(O)cc(-c3c(F)cc4c(N(C)C)nc(OC[C@@]56CCCN5C[C@H](F)C6)nc4c3F)c12]

### 4-(4-(dimethylamino)-6,8-difluoro-2-(((2R,7aS)-2-fluorotetrahydro-1H-pyrrolizin-7a(5H₁)-yl)methoxy)quinazolin-7-yl)-5-ethyl-6-fluoronaphthalen-2-ol

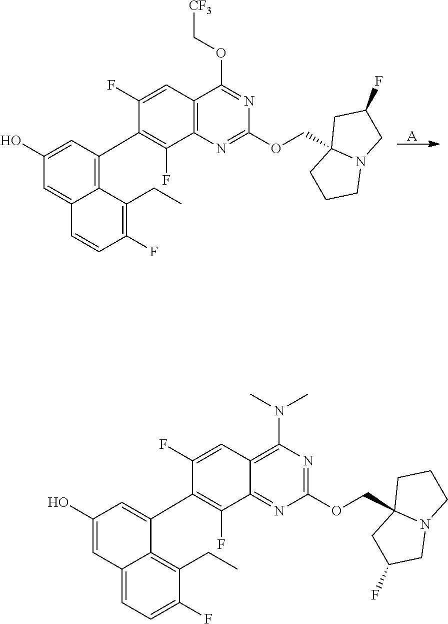

Step A: 4-(4-(dimethylamino)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol: A mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol in N-methylmethanamine (1 M, 1.64 mL, 5.0 equiv) was stirred at 60° C. for 12 hours. The mixture was concentrated and purified with reversed-phase HPLC(column: Waters Xbridge 150×25 mm×5 μm; mobile phase: [water (NH₄HCO₃)−ACN]; B %: 55%-85% over 8 min) to afford the title compound (41.9 mg, 23% yield) as yellow solid; SFC: “Column: Chiralpak IC-3 50×4.6 mm I.D., 3 μm Mobile phase: Phase A for CO₂, and Phase B for IPA (0.05% DEA); Gradient elution: 40% IPA (0.05% DEA) in CO₂ Flow rate: 3 mL/min; Detector: PDA; Column Temp: 35C; Back Pressure: 100 Bar. ¹H NMR (400 MHz, DMSO-d₆) δ=9.97 (s, 1H), 7.91 (d, J=10.4 Hz, 1H), 7.78 (dd, J=6.0, 8.4 Hz, 1H), 7.42-7.32 (m, 2H), 7.05-6.98 (m, 1H), 5.44-5.11 (m, 1H), 4.10 (dd, J=4.0, 10.4 Hz, 1H), 4.04-3.96 (m, 1H), 3.45-3.35 (m, 6H), 3.15-2.98 (m, 3H), 2.83-2.80 (m, 1H), 2.47-2.28 (m, 2H), 2.19-1.95 (m, 3H), 1.88-1.69 (m, 3H), 0.75 (t, J=7.2 Hz, 3H), LCMS (ESI, M+1): m/z=555.3.

### Example 61

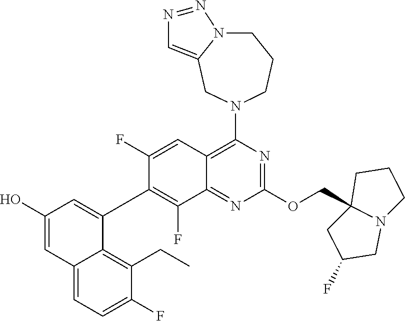 [SMILES: CCc1c(F)ccc2cc(O)cc(-c3c(F)cc4c(N5CCCn6nncc6C5)nc(OC[C@@]56CCCN5C[C@H](F)C6)nc4c3F)c12]

### 4-(4-(7,8-dihydro-4H-[1,2,3]triazolo[1,5-a][1,4]diazepin-5(6H)-yl)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol

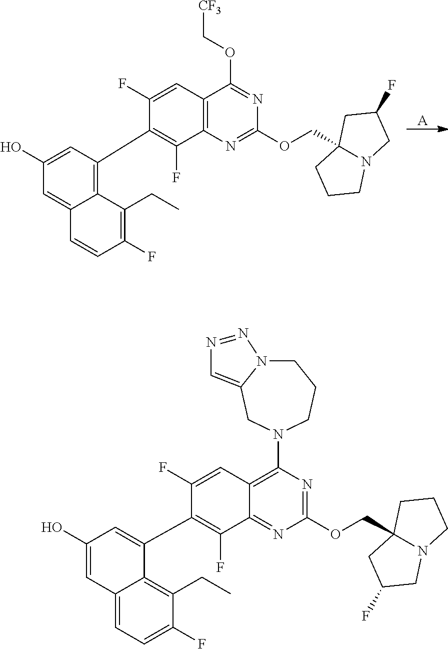

Step A. 4-(4-(7,8-dihydro-4H-[1,2,3]triazolo[1,5-a][1,4]diazepin-5(6H)-yl)-6,8-difluoro-2-(((2R,7aS)-2-fluorotetrahydro-1H-pyrrolizin-7a(5H)yl)methoxy)guinazolin-7-yl)-5-ethyl-6-fluoronaphthalen-2-ol: To a solution of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (200 mg, 1.0 equiv) and 5,6,7,8-tetrahydro-4H-triazolo[1,5-a][1,4]diazepine (90.7 mg, 2.0 equiv) in DMF (0.5 mL) was added DIEA (127 mg, 3.0 equiv). The mixture was stirred at 90° C. for 12 hours. The mixture was poured into water (2 mL) and filtered. The filtrate was extracted with ethyl acetate (3×10 mL). The organic phase was dried over Na₂SO₄ and concentrated under vacuum. The crude product was purified with reversed phase flash [Waters Xbridge 150×25 mm×5 um; mobile phase: A:water (NH₄HCO₃); B: ACN; B %: 40%-70% over 8.5 min] to afford the title compound (7.74 mg, 3.4% yield) as white solid; ¹H NMR (400 MHz, METHANOL-d₄) δ=7.83-7.76 (m, 1H), 7.72-7.63 (m, 2H), 7.31-7.20 (m, 2H), 7.00-6.95 (m, 1H), 5.39-5.09 (m, 3H), 4.79-4.71 (m, 2H), 4.44-4.27 (m, 2H), 4.23-4.12 (m, 2H), 3.29-3.15 (m, 3H), 3.07-2.96 (m, 1H), 2.63-2.50 (m, 1H), 2.46-2.34 (m, 3H), 2.31-2.07 (m, 3H), 2.04-1.86 (m, 3H), 0.84-0.75 (m, 3H); LCMS (ESI, M+1): m/z=648.4

### Example 62

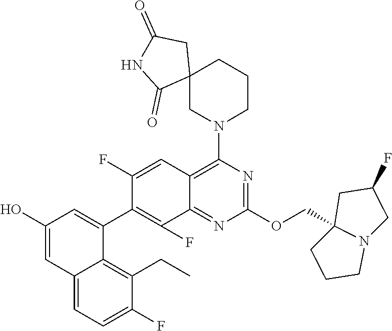 [SMILES: CCc1c(F)ccc2cc(O)cc(-c3c(F)cc4c(N5CCCC6(CC(=O)NC6=O)C5)nc(OC[C@@]56CCCN5C[C@H](F)C6)nc4c3F)c12]

### 7-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-2,7-azaspiro[4.5]decane-1,3-dione

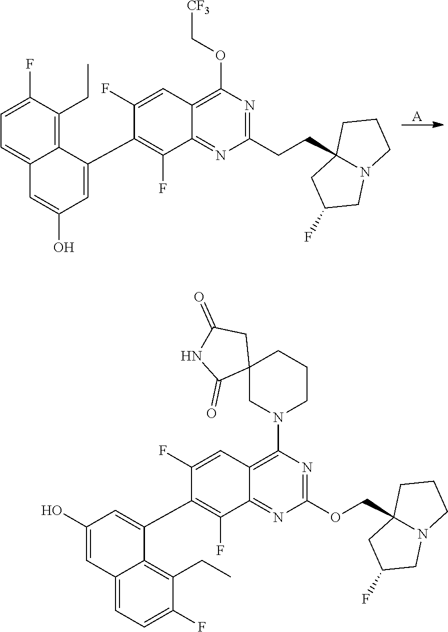

Step A. 7-(7-(8-ethyl-7-fluoro-3-hydroxynaphtalene-1-yl-6,8-difluoro-2-((2R,7aS)-2-fluorotetrahydro-1H-pyrrolizin-7a(5H₁)-yl)methoxy)guinazolin-4-yl)-2,7-diazaspiro[4.5]decane-1,3-dione: To a mixture of 4-(6,8-difluoro-2-(((2R,7aS)-2-fluorohexahydro-1H-pyrrolizin-7a-yl)methoxy)-4-(2,2,2-trifluoroethoxy)quinazolin-7-yl)-5-ethyl-6-fluoronaphthalen-2-ol (200 mg, 1.0 equiv), 2,7-diazaspiro[4.5]decane-1,3-dione (66.2 mg, 1.2 equiv) and 4 Å molecular sieve (50.0 mg, 1.0 equiv) in MeCN (1.0 mL) and DMF (1.0 mL) was added K₃PO₄ (209 mg, 3.0 equiv). The mixture was stirred at 40° C. for 12 hrs. The reaction mixture was filtered and purified by prep-HPLC [column: Waters Xbridge 150×25 mm×5 um; mobile phase: [water (ammonia hydroxide v/v)−ACN]; B %: 44%-74%,9 min] and lyophilized to afford the title compound (16.8 mg, 7% yield) as yellow solid; ¹H NMR (400 MHz, METHANOL-d₄) δ=7.67 (dd, J=6.0, 8.8 Hz, 1H), 7.63-7.58 (m, 1H), 7.31-7.28 (m, 1H), 7.27-7.21 (m, 1H), 7.00-6.97 (m, 1H), 5.40-5.21 (m, 1H), 4.42 (br t, J=12.0 Hz, 1H), 4.34-4.13 (m, 3H), 3.67-3.53 (m, 2H), 3.27-3.11 (m, 3H), 3.04-2.91 (m, 2H), 2.72-2.64 (m, 1H), 2.61-2.52 (m, 1H), 2.47-2.37 (m, 1H), 2.24 (br s, 1H), 2.22-2.07 (m, 3H), 2.06-1.83 (m, 6H), 0.86-0.73 (m, 3H); LCMS (ESI, M+1): m/z=678.5.

### Example 63

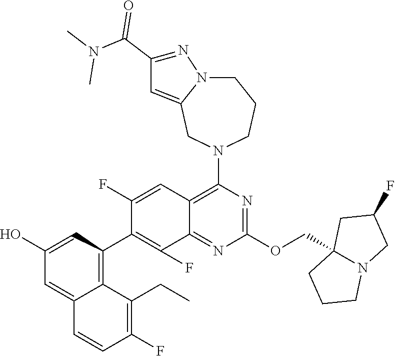

### 5-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide

### Example 64

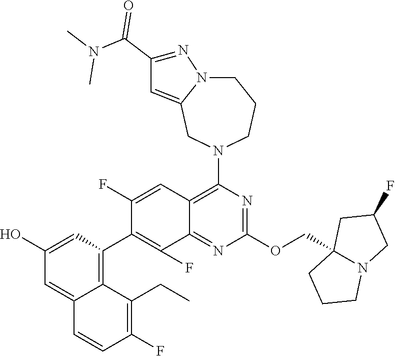

### 5-((S)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide

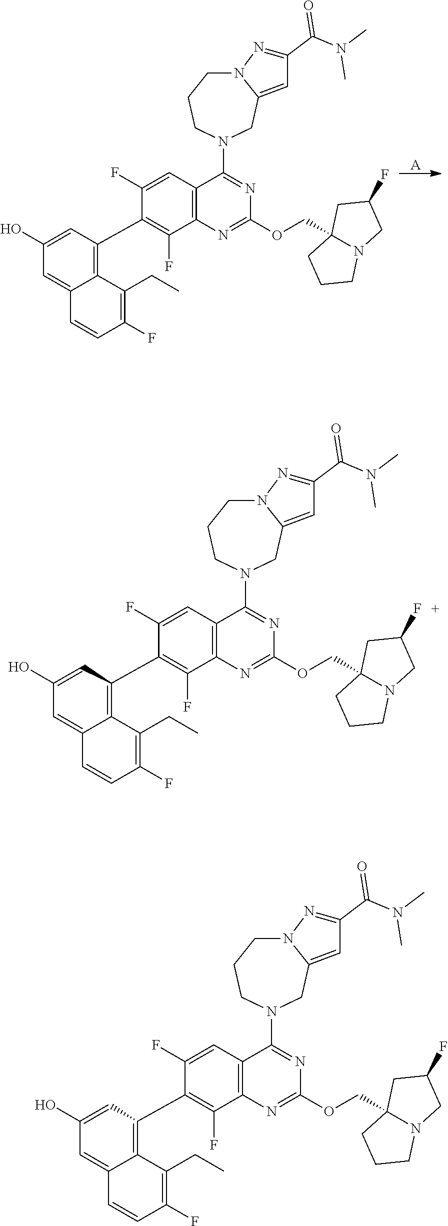

Step A: 5-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide and 5-((S)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide: 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide was separated by SFC (column: DAICEL CHIRALPAK IC (250 mm×30 mm, 10 m); mobile phase: [IPA−ACN]; B %: 40%-40% over 7.0 min), and then purified by prep-HPLC (column: Waters Xbridge 150×25 mm×5 m; Phase A: water (NH₄HCO₃); Phase B: ACN, B %: 48%-78% over 10 min) to afford two peaks.

Example 63 (124 mg, 13% yield) as white solid; column: Chiralpak IC-3 50×4.6 mm I.D., 3 μm, mobile phase: [40% IPA+ACN (0.05% DEA)] in CO₂, flow rate: 3 mL/min, detector: 220 nm, tR: 1.289 min; ¹H NMR (400 MHz, DMSO-d₆) δ 7.78-7.63 (m, 2H), 7.34-7.19 (m, 2H), 6.98 (d, J=2.8 Hz, 1H), 6.70 (s, 1H), 5.40-5.21 (m, 1H), 5.20-5.04 (m, 2H), 4.58-4.49 (m, 2H), 4.41-4.08 (m, 4H), 3.37-3.33 (m, 3H), 3.27-3.13 (m, 3H), 3.12-3.05 (m, 3H), 3.04-2.96 (m, 1H), 2.67-2.51 (m, 1H), 2.45-2.31 (m, 3H), 2.27-2.06 (m, 3H), 2.05-1.82 (m, 3H), 0.86-0.73 (m, 3H); LCMS (ESI, M+1): m/z=718.5.

Example 64 (148 mg, 16% yield) as white solid; column: Chiralpak IC-3 50×4.6 mm I.D., 3 μm, mobile phase: phase A: 40% IPA, phase B: ACN (0.05% DEA) in CO₂, flow rate: 3 mL/min, detector: 220 nm, tR: 1.979 min; ¹H NMR (400 MHz, DMSO-d₆) δ 7.77-7.64 (m, 2H), 7.33-7.20 (m, 2H), 6.98 (d, J=2.4 Hz, 1H), 6.70 (s, 1H), 5.40-5.22 (m, 1H), 5.21-5.06 (m, 2H), 4.64-4.43 (m, 2H), 4.40-4.15 (m, 4H), 3.35 (s, 3H), 3.27-3.13 (m, 3H), 3.08 (s, 3H), 3.04-2.97 (m, 1H), 2.63-2.51 (m, 1H), 2.45-2.33 (m, 3H), 2.25-2.05 (m, 3H), 2.04-1.84 (m, 3H), 0.80 (t, J=7.6 Hz, 3H); LCMS [ESI, M+1]: m/z=718.5.

### Example 65

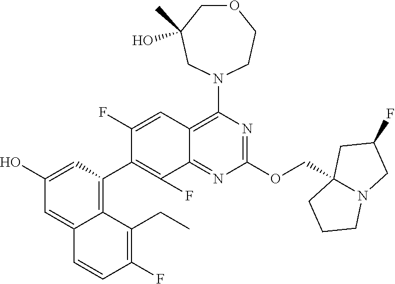

### (S)-4-((S)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol

### Example 66

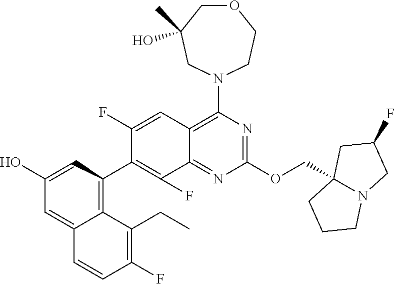

### (S)-4-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol

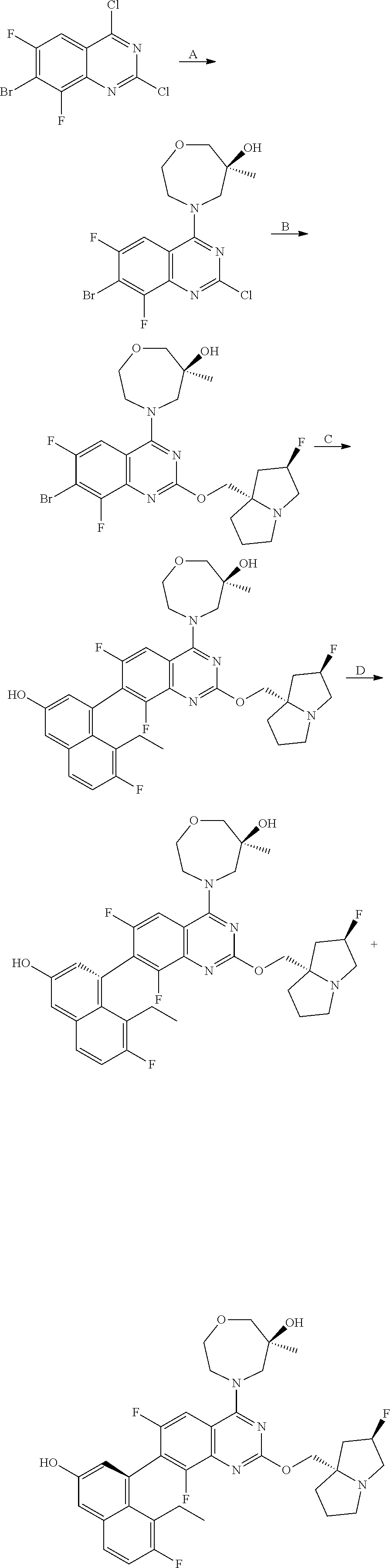

Step A. (S)-4-(7-bromo-2-chloro-6,8-difluoroquinazolin-4-yl)-6-methyl-1,4-oxazepan-6-ol: To a solution of 7-bromo-2,4-dichloro-6,8-difluoroquinazoline (12.0 g, 1.0 equiv), 4 Å molecular sieves (2 g) and DIEA (19.8 g, 4.0 equiv) in DCM (100 mL) was added a solution of (S)-6-methyl-1,4-oxazepan-6-ol (5.77 g, 0.9 equiv, HCl) in DCM (20 mL) at 0° C. The solution was diluted with water (50 mL) and extracted with DCM (50 mL×3). The combined organic layers were dried over Na₂SO₄, filtered and concentrated under vacuum and triturated with petroleum ether: ethyl acetate=5:1 at 15° C. for 15 minutes to afford the tittle compound (9.6 g, 56% yield) as yellow solid. LCMS (ESI, M+1, M+3): m/z=408.0, 410.0.

Step B. (S)-4-(7-bromo-6,8-difluoro-2-(((2R,7aS)-2-fluorohexahydro-1H-pyrrolizin-7a-yl)methoxy)guinazolin-4-yl)-6-methyl-1,4-oxazepan-6-ol: A mixture of (S)-4-(7-bromo-2-chloro-6,8-difluoroquinazolin-4-yl)-6-methyl-1,4-oxazepan-6-ol (3.20 g, 1.0 equiv) and ((2R,7aS)-2-fluorohexahydro-1H-pyrrolizin-7a-yl)methanol (16 mL) was stirred at 90° C. for 36 hours. The solution was diluted with MeOH (50 mL) and purified by reversed phase flash (C18, 0.1% formic acid condition) to afford the title compound (7.8 g, 3 batches, 57% yield) as yellow solid; LCMS (ESI, M+1, M+3): m/z=531.0, 533.2.

Step C. (6S)-4-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol: A mixture of (S)-4-(7-bromo-6,8-difluoro-2-(((2R,7aS)-2-fluorohexahydro-1H-pyrrolizin-7a-yl)methoxy)quinazolin-4-yl)-6-methyl-1,4-oxazepan-6-ol (7.50 g, 1.0 equiv), 5-ethyl-6-fluoro-4-(4,4,5,5-tetramethyl-1,3,2-dioxaborolan-2-yl)naphthalen-2-ol (5.80 g, 1.3 equiv), Ad2nBuP-Pd-G3 (1.03 g, 0.1 equiv) and K₃PO₄ (1.5 M in water, 28.2 mL, 3.0 equiv) in methoxycyclopentane (90 mL) was degassed and purged with N₂ for 3 times, and then the mixture was stirred at 90° C. for 2 hours under N₂ atmosphere. The mixture was diluted with water (50 mL), and extracted with ethyl acetate (3×50 mL). The combined organic layers were dried over Na₂SO₄, filtered and concentrated and purified by reversed phase flash [C18, 0.1% formic acid condition] to afford the title compound (6.3 g, 48% yield) as yellow solid; SFC condition: Chiralcel OJ-3 50×4.6 mm I.D., 3 um; Mobile phase: Phase A: CO₂, Phase B MeOH(0.05% DEA); Gradient elution: MeOH (0.05% DEA) in CO₂ from 5% to 40%; Flow rate: 3 mL/min;Detector: PDA; Column Temp: 35C; Back Pressure: 100 Bar; LCMS (ESI, M+1): m/z=641.3.

Step D. (S)-4-((S)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol and (S)-4-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-6-methyl-1,4-oxazepan-6-ol: The title compounds were separated by SFC (DAICEL CHIRALCEL OJ (250 mm 50 mm,10 μm); mobile phase A: water, B:(0.1% NH₃H₂O in MeOH), B %: 30%-30% over 2.5 min) to afford two peaks.

Example 65 (723 mg, 10% yield). SFC condition: Chiralcel OJ-3 50×4.6 mm I.D., 3 um; Mobile phase: Phase A: CO₂, Phase B: MeOH (0.05% DEA); Gradient elution: MeOH (0.05% DEA) in CO₂ from 5% to 40%; Flow rate: 3 mL/min; Detector: PDA; Column Temp: 35C; Back Pressure: 100 Bar; ¹H NMR (400 MHz, methanol-d₄) δ=8.08 (dd, J=1.6, 10.4 Hz, 1H), 7.67 (dd, J=6.0, 9.2 Hz, 1H), 7.29 (d, J=2.4 Hz, 1H), 7.24 (t, J=9.2 Hz, 1H), 6.97 (d, J=2.4 Hz, 1H), 5.42-5.19 (m, 1H), 4.47-4.40 (m, 1H), 4.37-4.18 (m, 3H), 4.10-3.96 (m, 2H), 3.91-3.80 (m, 2H), 3.71-3.59 (m, 2H), 3.29-3.13 (m, 3H), 3.05-2.97 (m, 1H), 2.64-2.53 (m, 1H), 2.49-2.40 (m, 1H), 2.38-2.24 (m, 1H), 2.23-2.08 (m, 2H), 2.03-1.82 (m, 3H), 1.24 (s, 3H), 0.82 (t, J=7.2 Hz, 3H); LCMS (ESI, M+3): m/z=641.3.

Example 66 (744 mg, 10% yield). SFC condition: Chiralcel OJ-3 50×4.6 mm I.D., 3 um; Mobile phase: Phase A: CO₂, Phase B: MeOH (0.05% DEA); Gradient elution: MeOH (0.05% DEA) in CO₂ from 5% to 40%; Flow rate: 3 mL/min; Detector: PDA; Column Temp: 35C; Back Pressure: 100 Bar; ¹H NMR (400 MHz, METHANOL-d₄) δ=8.11 (dd, J=1.2, 10.4 Hz, 1H), 7.67 (dd, J=6.0, 9.2 Hz, 1H), 7.28 (d, J=2.8 Hz, 1H), 7.24 (t, J=9.2 Hz, 1H), 6.98 (d, J=2.4 Hz, 1H), 5.41-5.17 (m, 1H), 5.52-4.44 (m, 1H), 4.37-4.19 (m, 3H), 4.06-3.95 (m, 2H), 3.88-3.78 (m, 2H), 3.73-3.57 (m, 2H), 3.28-3.13 (m, 3H), 3.05-2.96 (m, 1H), 2.61-2.48 (m, 1H), 2.45-2.35 (m, 1H), 2.35-2.16 (m, 2H), 2.16-2.07 (m, 1H), 2.03-1.82 (m, 3H), 1.26 (s, 3H), 0.78 (t, J=7.2 Hz, 3H)); LCMS [ESI, M+1]: m/z=641.3.

### Example 67

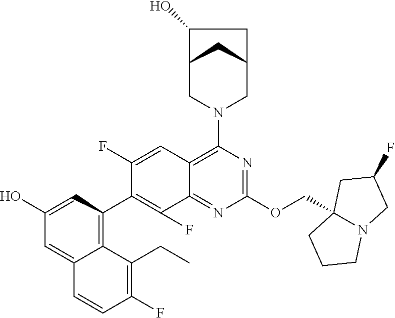

### (1R,5R,6R)-3-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-azabicyclo[3.2.1]octan-6-ol

### Example 68

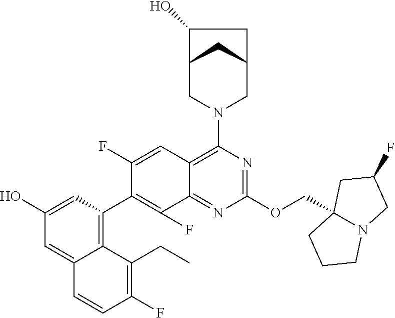

### (1R,5R,6R)-3-((S)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-azabicyclo[3.2.1]octan-6-ol

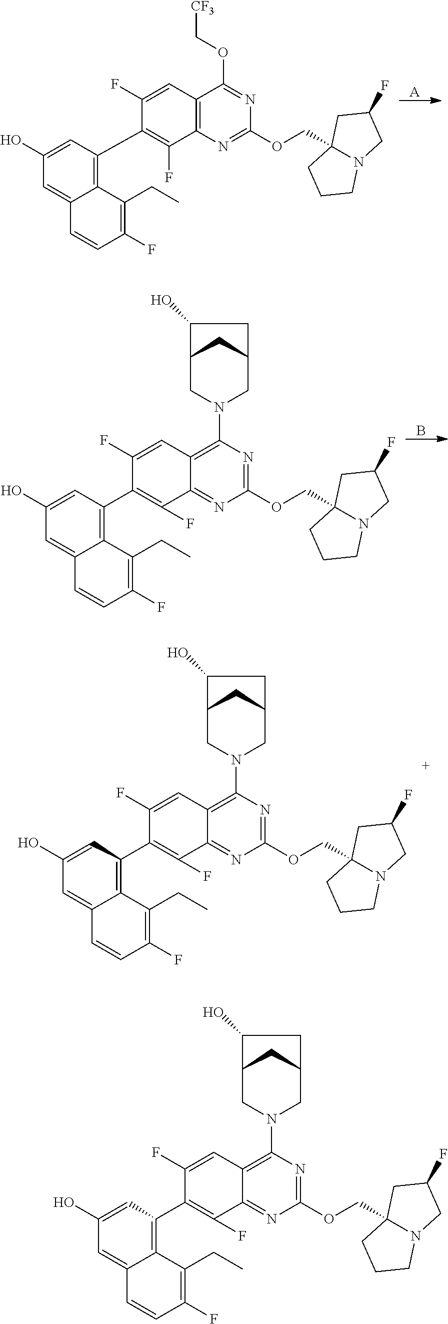

Step A: (1R,5R,6R)-3-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)guinazolin-4-yl)-3-azabicyclo[3.2.1]octan-6-ol: To a mixture of 4-(6,8-difluoro-2-(((2R,7aS)-2-fluorotetrahydro-1H-pyrrolizin-7a(5H)-yl)methoxy)-4-(2,2,2-trifluoroethoxy)quinazolin-7-yl)-5-ethyl-6-fluoronaphthalen-2-ol (1.0 g, 1.0 equiv), (1R,5R,6R)-3-azabicyclo[3.2.1]octan-6-ol (625.97 mg, 3.0 equiv) in DMF (2 mL) was added DIEA (636 mg, 3.0 equiv) and 4 Å molecular sieve (0.5 g). The mixture was at 60° C. for 12 hours. Water (10 mL) was added to the reaction mixture, and extracted with ethyl acetate (2×10 mL). The combined organic layers were washed with brine (20 mL), dried over Na₂SO₄, and concentrated under reduced pressure. The residue was purified by reversed phase flash [C18, 0.1% formic acid condition] to afford the title compound (0.82 g, 70% yield) as yellow solid; LCMS (ESI, M+1): m/z=637.5.

Step B: (1R,5R,6R)-3-((R)-7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)guinazolin-4-yl)-3-azabicyclo[3.2.1]octan-6-ol and (1R,5R,6R)-3-((S)-7-(8-ethyl-7-fluoro-3-hydroxynaphthalen-1-yl)-6,8-difluoro-2-(2R,7aS)-2-fluorohexahydro-1H-pyrrolizin-7a-yl)methoxy)quinazolin-4-yl)-3-azabicyclo[3.2.1]octan-6-ol: The title compounds were separated by SFC (column: Waters Xbridge 150×25 mm×5 μm; mobile phase: [water(NH₄HCO₃)−ACN]; B %: 57%-87% over 8 min) to afford two peaks.

Example 67 (179 mg, 21% yield) as yellow solid. SFC: 100% ee, “Column: Chiralpak IC-3 50×4.6 mm I.D., 3 μm Mobile phase: Phase A for CO₂, and Phase B for EtOH (0.05% DEA); Gradient elution: EtOH (0.05% DEA) in CO₂ from 5% to 40% Flow rate: 3 mL/min; Detector: PDA; Column Temp: 35C; Back Pressure: 100 Bar”. ¹H NMR (400 MHz, methanol-d₄) δ=8.05-7.92 (m, 1H), 7.67 (dd, J=6.0, 8.4 Hz, 1H), 7.36-7.19 (m, 2H), 6.97 (d, J=2.0 Hz, 1H), 5.39-5.21 (m, 1H), 4.70 (d, J=12.0 Hz, 1H), 4.69-4.62 (m, 1H), 4.37-4.16 (m, 3H), 3.55-3.52 (m, 1H), 3.48-3.42 (m, 1H), 3.23-3.19 (m, 3H), 3.04-2.98 (m, 1H), 2.62-2.55 (m, 1H), 2.47-2.31 (m, 3H), 2.31-2.10 (m, 5H), 1.98-1.75 (m, 4H), 1.55-1.42 (m, 1H), 0.80 (t, J=7.2 Hz, 3H); LCMS (ESI, M+1): m/z=637.3.

Example 68 (179 mg, 21% yield) as yellow solid. SFC: 98.9% ee, Column: Chiralpak IC-3 50×4.6 mm I.D., 3 μm Mobile phase: Phase A for CO₂, and Phase B for EtOH (0.05% DEA); Gradient elution: EtOH (0.05% DEA) in CO₂ from 5% to 40% Flow rate: 3 mL/min; Detector: PDA; Column Temp: 35C; Back Pressure: 100 Bar”. ¹H NMR (400 MHz, methanol-d₄) δ=8.08-7.93 (m, 1H), 7.67 (dd, J=6.0, 9.2 Hz, 1H), 7.37-7.16 (m, 2H), 6.97 (d, J=2.4 Hz, 1H), 5.40-5.20 (m, 1H), 4.76-4.54 (m, 2H), 4.40-4.15 (m, 3H), 3.57 (d, J=12.0 Hz, 1H), 3.49-3.41 (m, 2H), 3.25-3.16 (m, 2H), 3.08-2.95 (m, 1H), 2.62-2.51 (m, 1H), 2.50-2.29 (m, 3H), 2.28-2.11 (m, 4H), 1.99-1.77 (m, 4H), 1.66-1.58 (m, 1H), 1.57-1.43 (m, 1H), 0.80 (t, J=8 Hz, 3H); LCMS (ESI, M+1): m/z=637.4.

### Example 69

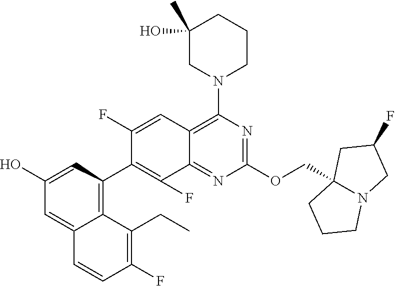

### (R)-1-((R)-7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol

### Example 70

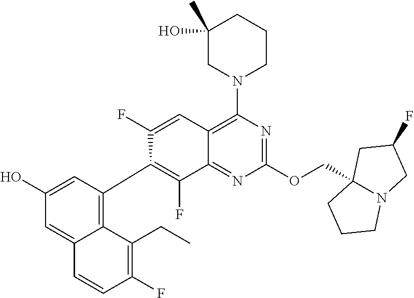

### (R)-1-((S)-7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol

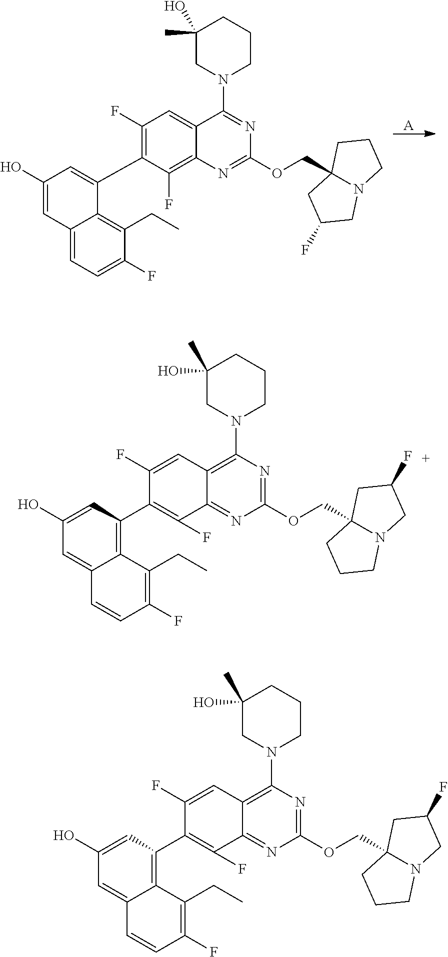

Step A. (R)-1-((R)-7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)guinazolin-4-yl)-3-methylpiperidin-3-ol and (R)-1-((S)-7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-methylpiperidin-3-ol: The title compounds were separated by SFC [condition: column: DAICEL CHIRALPAK AD (250 mm×50 mm,10 um); mobile phase: [0.1% NH₃H₂O IPA]; B %: 55%-55%,4.1 minutes] to afford two peaks.

Example 69 (6.85 g, 20% yield) as a yellow solid; ¹H NMR (400 MHz, methanol-d4) δ=7.80 (d, J=9.6 Hz, 1H), 7.67 (dd, J=6.0, 9.2 Hz, 1H), 7.29 (d, J=2.4 Hz, 1H), 7.24 (t, J=9.2 Hz, 1H), 6.98 (d, J=2.8 Hz, 1H), 5.44-5.15 (m, 1H), 4.34-4.18 (m, 3H), 4.05 (br d, J=13.2 Hz, 1H), 3.46-3.35 (m, 2H), 3.28-3.13 (m, 3H), 3.06-2.95 (m, 1H), 2.63-2.51 (m, 1H), 2.47-2.11 (m, 5H), 2.03-1.71 (m, 6H), 1.29 (s, 3H), 0.81 (t, J=7.2 Hz, 3H); LCMS (ESI, M+1): m/z=625.5.

Example 70 (6.02 g, 18% yield) as a yellow solid; ¹H NMR (400 MHz, methanol-d4) δ=7.76 (br d, J=8.8 Hz, 1H), 7.67 (dd, J=6.0, 9.2 Hz, 1H), 7.29 (d, J=2.4 Hz, 1H), 7.24 (t, J=9.2 Hz, 1H), 6.98 (d, J=2.4 Hz, 1H), 5.45-5.16 (m, 1H), 4.33-4.15 (m, 3H), 4.05 (br d, J=13.2 Hz, 1H), 3.50 (d, J=13.2 Hz, 1H), 3.42-3.34 (m, 1H), 3.29-3.12 (m, 3H), 2.97-3.03 (m, 1H), 2.63-2.50 (m, 1H), 2.45-2.10 (m, 5H), 2.02-1.70 (m, 6H), 1.25 (s, 3H), 0.80 (t, J=7.2 Hz, 3H); LCMS (ESI, M+1): m/z=625.3.

### Example 71

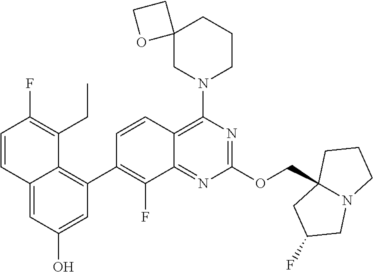 [SMILES: CCc1c(F)ccc2cc(O)cc(-c3ccc4c(N5CCCC6(CCO6)C5)nc(OC[C@@]56CCCN5C[C@H](F)C6)nc4c3F)c12]

### 5-ethyl-6-fluoro-4-(8-fluoro-2-(((2R,7aS)-2-fluorohexahydro-1H-pyrrolizin-7a-yl)methoxy)-4-(1-oxa-6-azaspiro[3.5]nonan-6-yl)quinazolin-7-yl)naphthalen-2-ol

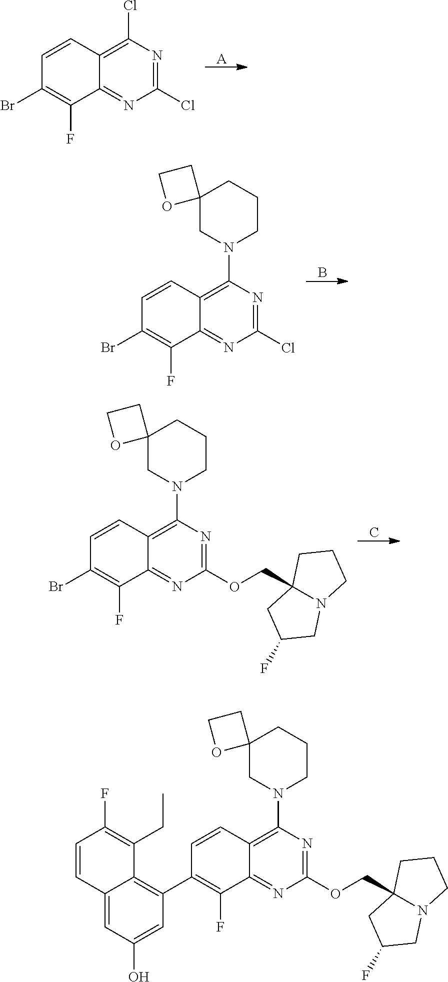

Step A. 6-(7-bromo-2-chloro-8-fluoroquinazolin-4-yl)-1-oxa-6-azaspiro[3.5]nonane: To a mixture of 7-bromo-2,4-dichloro-8-fluoro-quinazoline (300 mg, 1.0 equiv) and 1-oxa-8-azaspiro[3.5]nonane; oxalic acid (349 mg, 1.0 equiv) in DCM (10 mL) was added DIEA (393 mg, 3.0 equiv). The reaction mixture was stirred at −40° C. for 2 hours under nitrogen atmosphere. The mixture was concentrated and purified by flash silica gel chromatography [SiO₂, Petroleum ether/Ethyl acetate=3/1] to afford the title compound (300 mg, 69% yield) as yellow solid; ¹H NMR (400 MHz, CHLOROFORM-d) δ=7.76 (d, J=9.2 Hz, 1H), 7.55 (dd, J=6.4, 8.8 Hz, 1H), 4.57 (t, J=8.0 Hz, 2H), 4.25 (d, J=13.2 Hz, 1H), 4.03 (td, J=4.4, 13.2 Hz, 1H), 3.66 (d, J=13.2 Hz, 1H), 3.41 (ddd, J=2.8, 9.6, 13.2 Hz, 1H), 2.43 (t, J=8.0 Hz, 2H), 2.32-2.18 (m, 1H), 2.10-1.97 (m, 1H), 1.93-1.84 (m, 1H), 1.83-1.73 (m, 1H); LCMS (ESI, M+1): m/z=388.0.

Step B. 6-(7-bromo-8-fluoro-2-(((2R,7aS)-2-fluorohexahydro-1H-pyrrolizin-7a-yl)methoxy)quinazolin-4-yl)-1-oxa-6-azaspiro[3.5]nonane: A mixture of 8-(7-bromo-2-chloro-8-fluoro-quinazolin-4-yl)-1-oxa-8-azaspiro[3.5]nonane (110 mg, 1.0 equiv) and [(2R,8S)-2-fluoro-1,2,3,5,6,7-hexahydropyrrolizin-8-yl]methanol (453 mg, 10 equiv) in DMSO (0.05 mL) was stirred at 90° C. for 12 hours under N₂ atmosphere. The mixture was concentrated and purified with prep-HPLC [FA condition; column: Phenomenex Synergi C18 150×25 mm×10 μm; mobile phase: [water (FA)−ACN]; B %: 9%-39%, 10 minutes] to afford the title compound (75.0 mg, 50% yield) as yellow solid. ¹H NMR (400 MHz, CHLOROFORM-d) δ=8.32 (s, 1H), 7.68 (dd, J=1.2, 8.8 Hz, 1H), 5.47-5.29 (m, 1H), 4.57 (t, J=7.6 Hz, 2H), 4.47 (t, J=2.8 Hz, 2H), 4.14 (br dd, J=4.4, 13.2 Hz, 2H), 4.09-3.97 (m, 3H), 3.76-3.58 (m, 4H), 3.42-3.29 (m, 2H), 3.13-3.05 (m, 1H), 2.41 (br s, 2H), 2.34 (br s, 1H), 2.23-2.17 (m, 1H), 2.09 (br d, J=5.6 Hz, 2H), 1.91-1.83 (m, 1H), 1.81-1.73 (m, 1H); LCMS [ESI, M+1]: m/z=511.0

Step C. 5-ethyl-6-fluoro-4-(8-fluoro-2-(((2R,7aS)-2-fluorohexahydro-1H-pyrrolizin-7a-yl)methoxy)-4-(1-oxa-6-azaspiro[3.5]nonan-6-yl)quinazolin-7-yl)naphthalen-2-ol: To a mixture of 5-ethyl-6-fluoro-4-(4,4,5,5-tetramethyl-1,3,2-dioxaborolan-2-yl)naphthalen-2-ol (37.2 mg, 1.2 equiv) and 6-(7-bromo-8-fluoro-2-(((2R,7aS)-2-fluorotetrahydro-1H-pyrrolizin-7a(5H)-yl)methoxy)quinazolin-4-yl)-1-oxa-6-azaspiro[3.5]nonane (50.0 mg, 1.0 equiv) in methoxycyclopentane (3.0 mL) and H₂O (0.6 mL) were added Ad₂nBuP Pd G3(cataCXium® A Pd G3) (7.10 mg, 0.1 equiv) and Cs₂CO₃ (96.0 mg, 3.0 equiv). The reaction mixture was degassed and purged with nitrogen for 3 times and stirred at 80° C. for 2 hours under N₂ atmosphere. The mixture was concentrated and purified with prep-HPLC [column: Waters Xbridge 150×25 mm×5 um; mobile phase: [water (ammonia hydroxide v/v)−ACN]; B %: 37%-67%, 9 minutes] to afford the title compound (13.5 mg, 25% yield, HCOOH salt) as a white solid. ¹H NMR (400 MHz, METHANOL-d₄) δ=8.54 (s, 1H), 8.02-7.92 (m, 1H), 7.70-7.60 (m, 1H), 7.40-7.28 (m, 1H), 7.27-7.18 (m, 2H), 6.96 (d, J=2.0 Hz, 1H), 5.50-5.24 (m, 1H), 4.58 (br s, 2H), 4.43-4.31 (m, 2H), 4.25-4.07 (m, 1H), 3.89-3.68 (m, 1H), 3.57-3.33 (m, 4H), 3.18-3.05 (m, 1H), 2.54-2.27 (m, 6H), 2.26-2.16 (m, 2H), 2.08 (br s, 3H), 2.01-1.88 (m, 2H), 1.80 (ddd, J=2.4, 4.0, 8.4 Hz, 1H), 0.78 (br t, J=7.2 Hz, 3H); LCMS [ESI, M+1]: m/z=619.1.

### Example 72

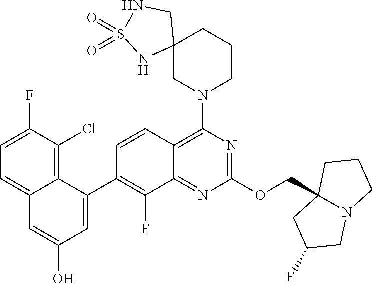 [SMILES: O=S1(=O)NCC2(CCCN(c3nc(OC[C@@]45CCCN4C[C@H](F)C5)nc4c(F)c(-c5cc(O)cc6ccc(F)c(Cl)c56)ccc34)C2)N1]

### 7-(7-(8-chloro-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-2-thia-1,3,7-triazaspiro[4.5]decane 2,2-dioxide

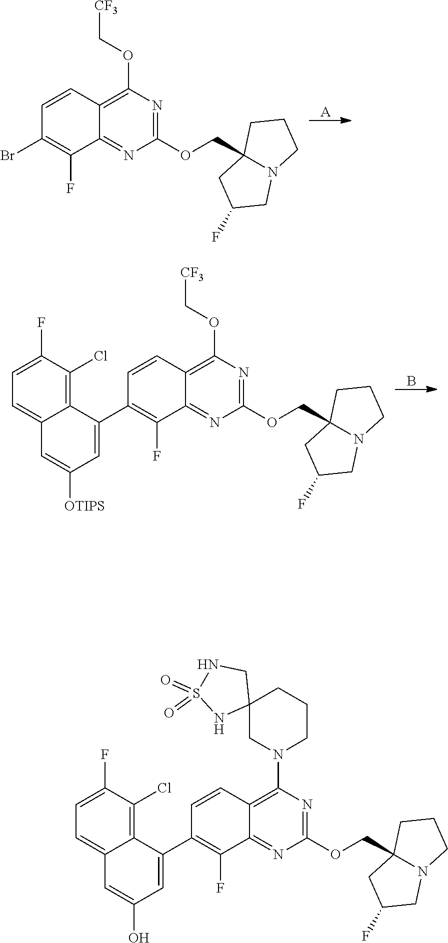

Step A. 7-(8-chloro-7-fluoro-3-((triisopropylsilyl)oxy)naphthalen-1-yl)-8-fluoro-2-(((2R,7aS)-2-fluorohexahydro-1H-pyrrolizin-7a-yl)methoxy)-4-(2,2,2-trifluoroethoxy)quinazoline: To a solution of 7-bromo-8-fluoro-2-(((2R,7aS)-2-fluorohexahydro-1H-pyrrolizin-7a-yl)methoxy)-4-(2,2,2-trifluoroethoxy)quinazoline (480 mg, 1.0 equiv) and (5-chloro-6-fluoro-4-trimethylstannyl-2-naphthyl)oxy-triisopropyl-silane (513 mg, 1.0 equiv) in DMAc (5 mL) was added CataCXium A Pd G3(72.5 mg, 0.1 equiv) under N₂. The reaction mixture was stirred at 90° C. for 5 hours under N₂ atmosphere. The reaction mixture was diluted with water (40 mL) and extracted with ethyl acetate (2×40 mL). The combined layers were washed with brine (40 mL), dried over sodium sulfate, concentrated and purified with reversed phase flash (C 18, 0.1% FA) to afford the title compound (140 mg, 30% yield) as colorless oil.

Step B. 7-(7-(8-chloro-7-fluoro-3-hydroxynaphthalen-1-yl)-8-fluoro-2-(((2R,7aS)-2-fluorotetrahydro-1H-pyrrolizin-7a(5H)-yl)methoxy)guinazolin-4-yl)-2-thia-1,3,7-triazaspiro[4.5]decane 2,2-dioxide: To a solution of 7-(8-chloro-7-fluoro-3-((triisopropylsilyl)oxy)naphthalen-1-yl)-8-fluor-2-(((2R,7aS)-2-fluorotetrahydro-1H-pyrrolizin-7a(5H)-yl)methoxy)-4-(2,2,2-trifluoroethoxy)quinazoline (60 mg, 1.0 equiv) and 2-thia-1,3,7-triazaspiro[4.5]decane 2,2-dioxide (53.2 mg, 3.5 equiv) in DMF (0.6 mL) were added 4 Å molecular sieve (7 g) and potassium phosphate (33.8 mg, 2.0 equiv). The reaction mixture was stirred at 60° C. for 12 hours under N₂ atmosphere. The mixture was diluted with water (10 mL) and extracted with ethyl acetate (2×10 mL). The combined organic layers were washed with brine (10 mL), dried over sodium sulfate, concentrated and purified with prep-HPLC (column: Phenomenex luna C18 150×25 mm×10 μm; A: water(FA);B: ACN, B %: 12%-42% over 2 min) to afford the title compound (36.0 mg) as yellow solid (FA salt). 1H NMR (400 MHz, METHANOL-d₄) δ=8.51 (br s, 1H), 8.01-7.90 (m, 1H), 7.78 (ddd, J=2.0, 5.6, 9.2 Hz, 1H), 7.37 (dt, J=1.6, 8.8 Hz, 1H), 7.34-7.29 (m, 2H), 7.09 (s, 1H), 5.60-5.31 (m, 1H), 4.64-4.47 (m, 2H), 4.47-4.32 (m, 1H), 4.30-4.16 (m, 1H), 3.82-3.50 (m, 5H), 3.43 (d, J=11.9 Hz, 1H), 3.29-3.19 (m, 2H), 2.64-2.36 (m, 2H), 2.36-2.27 (m, 1H), 2.25-2.13 (m, 2H), 2.12-1.96 (m, 3H), 1.95-1.81 (m, 2H); LCMS [ESI, M+1]: m/z=689.1.

### Example 73

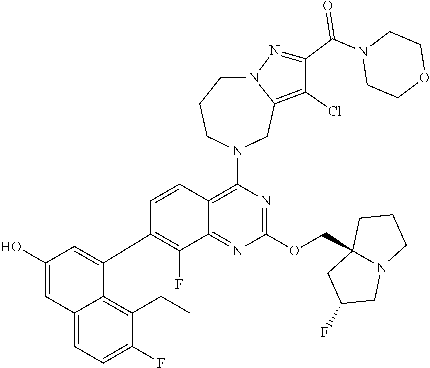 [SMILES: CCc1c(F)ccc2cc(O)cc(-c3ccc4c(N5CCCn6nc(C(=O)N7CCOCC7)c(Cl)c6C5)nc(OC[C@@]56CCCN5C[C@H](F)C6)nc4c3F)c12]

### (3-chloro-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(morpholino)methanone

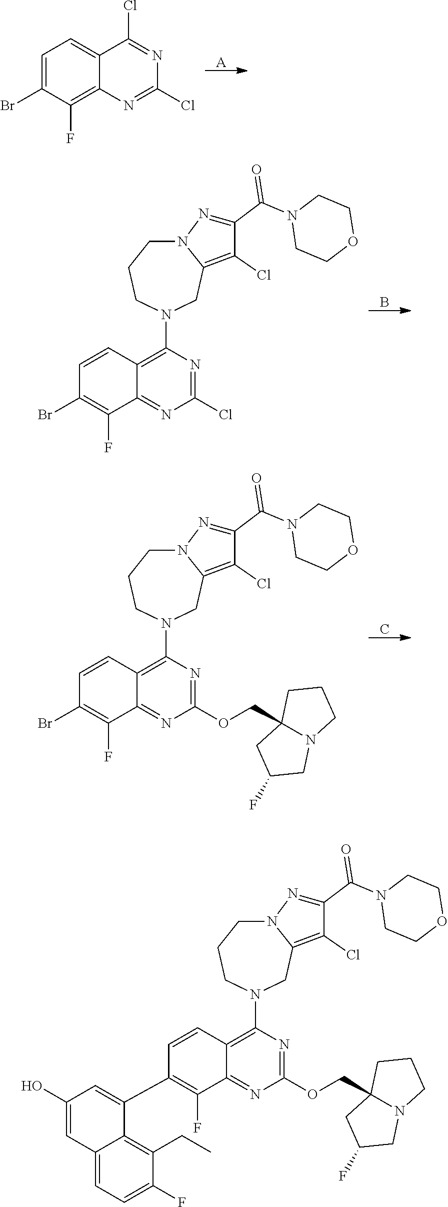

Step A. (5-(7-bromo-2-chloro-8-fluoroquinazolin-4-yl)-3-chloro-5,6,7,8-tetrahydro-4H-pyrazolo[1,5-a][1,4]diazepin-2-yl)(morpholino)methanone: To a solution of 7-bromo-2,4-dichloro-8-fluoroquinazoline (1.00 g, 1.0 equiv) and (3-chloro-5,6,7,8-tetrahydro-4H-pyrazolo[1,5-a][1,4]diazepin-2-yl)(morpholino)methanone(1.15 g, 1.2 equiv) in dichloromethane (10 mL) was added DIEA (1.31 g, 3.0 equiv). The reaction mixture was stirred at −40° C. for 0.5 hours. The reaction mixture was diluted with water (10 mL) and extracted with dichloromethane (4×15 mL). The combined organic layers were dried over anhydrous sodium sulfate, concentrated and slurried with acetonitrile (30 mL) at 20° C. for 20 minutes to afford the title compound (1.20 g, 64% yield) as white solid; LCMS (ESI, M+1): m/z=545.0.

Step B. (5-(7-bromo-8-fluoro-2-(((2R,7aS)-2-fluorohexahydro-1H-pyrrolizin-7a-yl)methoxy)guinazolin-4-yl)-3-chloro-5,6,7,8-tetrahydro-4H-pyrazolo[1,5-a][1,4]diazepin-2-yl)(morpholino)methanone: A mixture of (5-(7-bromo-2-chloro-8-fluoroquinazolin-4-yl)-3-chloro-5,6,7,8-tetrahydro-4H-pyrazolo[1,5-a][1,4]diazepin-2-yl)(morpholino)methanone (1.10 g, 1.0 equiv) and ((2R,7aS)-2-fluorohexahydro-1H-pyrrolizin-7a-yl)methanol (965 mg, 3.0 equiv) was stirred at 110° C. for 12 hours. The mixture was diluted with water (20 mL) and extracted with ethyl acetate (4×15 mL). The combined organic layers were washed with brine (30 mL), dried over anhydrous sodium sulfate, concentrated and purified with reversed phase flash chromatography [C18, 0.1% formic acid condition] to afford the title compound (100 mg, 7.1% yield) as yellow solid; LCMS (ESI, M+1): m/z=668.0.

Step C. (3-chloro-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(morpholino)methanone: To a mixture of (5-(7-bromo-8-fluoro-2-(((2R,7aS)-2-fluorohexahydro-1H-pyrrolizin-7a-yl)methoxy)quinazolin-4-yl)-3-chloro-5,6,7,8-tetrahydro-4H-pyrazolo[1,5-a][1,4]diazepin-2-yl)(morpholino)methanone (100 mg, 1.0 equiv), 5-ethyl-6-fluoro-4-(4,4,5,5-tetramethyl-1,3,2-dioxaborolan-2-yl)naphthalen-2-ol (71.1 mg, 1.5 equiv) and K₃PO₄ (95.5 mg, 3.0 equiv) in methoxycyclopentane (1.0 mL) and H₂O (0.3 mL) was added Ad₂nBup-Pd-G₃ (10.9 mg, 0.10 equiv) under nitrogen atmosphere. The reaction mixture was stirred at 90° C. for 2 hours. The reaction mixture was diluted with water (5 mL) and extracted with ethyl acetate (4×5 mL). The combined organic layers were washed with brine (10 mL), dried over anhydrous sodium sulfate, concentrated and purified with prep-HPLC [Waters Xbridge 150×25 mm×5 μm; A: water (NH₄HCO₃), B: ACN, B %: 51%-81% over 10 min] and prep-HPLC [Phenomenex C18 75×30 mm×3 μm; A: water (FA), B: ACN, B %: 18%-48% over 7 min] to afford the title compound (54.2 mg, 46% yield, HCOOH salt) as white solid; ¹H NMR (400 MHz, METHANOL-d₄) δ=7.92 (d, J=8.8 Hz, 1H), 7.68-7.64 (m, 1H), 7.38-7.34 (m, 1H), 7.28-7.19 (m, 2H), 6.94 (d, J=2.4 Hz, 1H), 5.53-5.33 (m, 1H), 5.21-5.03 (m, 2H), 4.47-4.30 (m, 6H), 3.75 (s, 4H), 3.67 (s, 4H), 3.65-3.40 (m, 3H), 3.26-3.15 (m, 1H), 2.61-2.31 (m, 6H), 2.30-2.22 (m, 1H), 2.19-2.08 (m, 2H), 2.03 (s, 1H), 0.79-0.75 (m, 3H); LCMS (ESI, M+1): m/z=776.3.

### Example 74

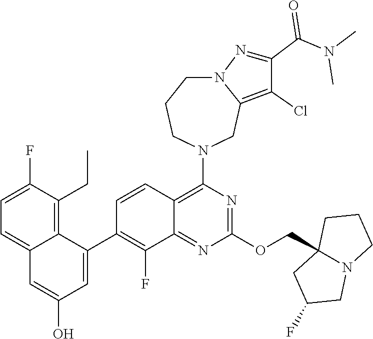 [SMILES: CCc1c(F)ccc2cc(O)cc(-c3ccc4c(N5CCCn6nc(C(=O)N(C)C)c(Cl)c6C5)nc(OC[C@@]56CCCN5C[C@H](F)C6)nc4c3F)c12]

### 3-chloro-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide

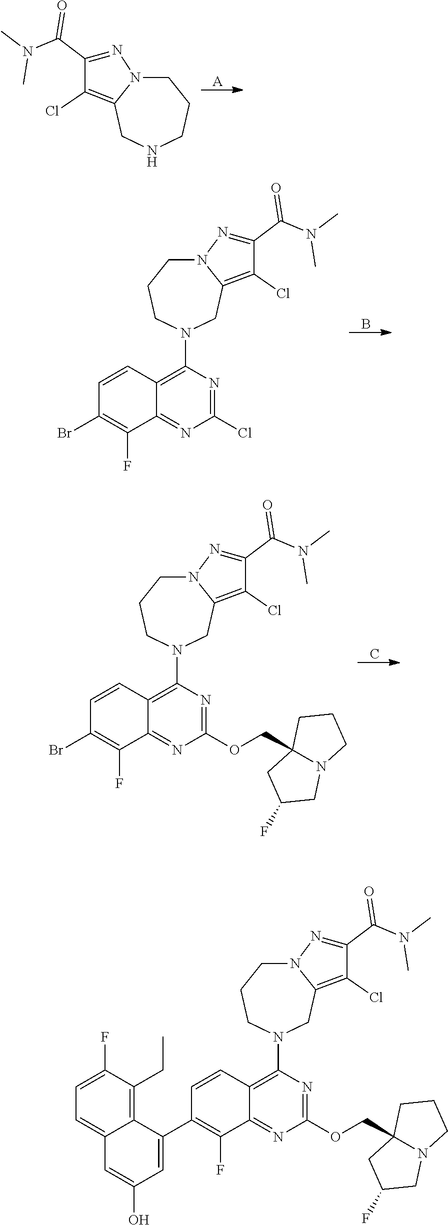

Step A. 5-(7-bromo-2-chloro-8-fluoroquinazolin-4-yl)-3-chloro-N,N-dimethyl-5,6,7,8-tetrahydro-4H-pyrazolo[1,5-a][1,4]diazepine-2-carboxamide: To a solution of 7-bromo-2,4-dichloro-8-fluoroquinazoline (950 mg, 1.0 equiv) and 3-chloro-N,N-dimethyl-5,6,7,8-tetrahydro-4H-pyrazolo[1,5-a][1,4]diazepine-2-carboxamide (779 mg, 1.0 equiv) in dichloromethane (9 mL) was added DIEA (2.07 g, 5.0 equiv) at −40° C. The reaction mixture was stirred at −40° C. for 1 hour. The reaction mixture was diluted with water (10 mL) and extracted with dichloromethane (3×10 mL). The combined organic layers were dried over anhydrous sodium sulfate, concentrated and triturated with acetonitrile (10 mL) at 20° C. for 20 minutes, filtered and concentrated to afford the title compound (1.13 g, 69% yield) as white solid; LCMS (ESI, M+1): m/z=503.1.

Step B. 5-(7-bromo-8-fluoro-2-(((2R,7aS)-2-fluorohexahydro-1H-pyrrolizin-7a-yl)methoxy)quinazolin-4-yl)-3-chloro-N,N-dimethyl-5,6,7,8-tetrahydro-4H-pyrazolo[1,5-a][1,4]diazepine-2-carboxamide: A mixture of 5-(7-bromo-2-chloro-8-fluoroquinazolin-4-yl)-3-chloro-N,N-dimethyl-5,6,7,8-tetrahydro-4H-pyrazolo[1,5-a][1,4]diazepine-2-carboxamide (500 mg, 1.0 equiv) and ((2R,7aS)-2-fluorohexahydro-1H-pyrrolizin-7a-yl)methanol (792 mg, 5.0 equiv) was stirred at 110° C. for 12 hours. The reaction mixture was cooled to 20° C. and diluted with water (5 mL). The crude product was slurried with H₂O (6 mL) at 20° C. for 20 minutes and triturated with acetonitrile (4 mL) at 20° C. for 20 minutes to afford the title compound (420 mg, 61% yield) as white solid; LCMS (ESI, M+1): m/z=626.1.

Step C. 3-chloro-5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide: To a mixture of 5-(7-bromo-8-fluoro-2-(((2R,7aS)-2-fluorohexahydro-1H-pyrrolizin-7a-yl)methoxy)quinazolin-4-yl)-3-chloro-N,N-dimethyl-5,6,7,8-tetrahydro-4H-pyrazolo[1,5-a][1,4]diazepine-2-carboxamide (150 mg, 1.0 equiv), 5-ethyl-6-fluoro-4-(4,4,5,5-tetramethyl-1,3,2-dioxaborolan-2-yl)naphthalen-2-ol (114 mg, 1.5 equiv) and K₃PO₄ (153 mg, 3.0 equiv) in methoxycyclopentane (1.5 mL) and H₂O (0.5 mL) was added Ad2nBuP-Pd-G3 (17.5 mg, 0.10 equiv). The reaction mixture was stirred at 90° C. for 2 hours under N₂ atmosphere. The reaction mixture was diluted with water (10 mL) and extracted with ethyl acetate (3×10 mL). The combined organic layers were dried over anhydrous sodium sulfate, concentrated and purified with reversed phase flash chromatography [C18, 0.1% formic acid condition] and prep-HPLC [Waters Xbridge 150×25 mm×5 μm; A: water (NH₄HCO₃), B: ACN, B %: 46%-76% over 8 min] to afford the title compound (30.3 mg, 17% yield) as white solid; ¹H NMR (400 MHz, METHANOL-d₄) δ=7.88 (d, J=8.8 Hz, 1H), 7.65 (dd, J=6.0, 9.2 Hz, 1H), 7.32 (dd, J=6.8, 8.4 Hz, 1H), 7.28-7.17 (m, 2H), 6.94 (d, J=2.4 Hz, 1H), 5.44-5.24 (m, 1H), 5.22-5.03 (m, 2H), 4.52-4.40 (m, 2H), 4.40-4.29 (m, 2H), 4.25 (s, 2H), 3.49-3.34 (m, 1H), 3.29-3.17 (m, 2H), 3.11 (d, J=12.4 Hz, 6H), 3.08-2.99 (m, 1H), 2.56-2.28 (m, 5H), 2.27-2.10 (m, 2H), 2.05-1.89 (m, 3H), 0.77 (dt, J=3.2, 7.2 Hz, 3H); LCMS (ESI, M+1): m/z=734.2.

### Example 75

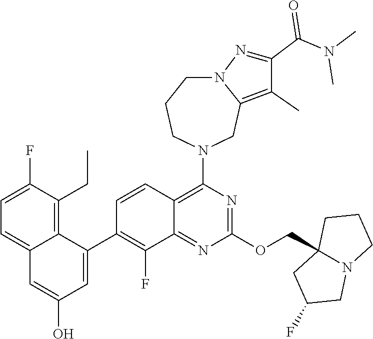 [SMILES: CCc1c(F)ccc2cc(O)cc(-c3ccc4c(N5CCCn6nc(C(=O)N(C)C)c(C)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide

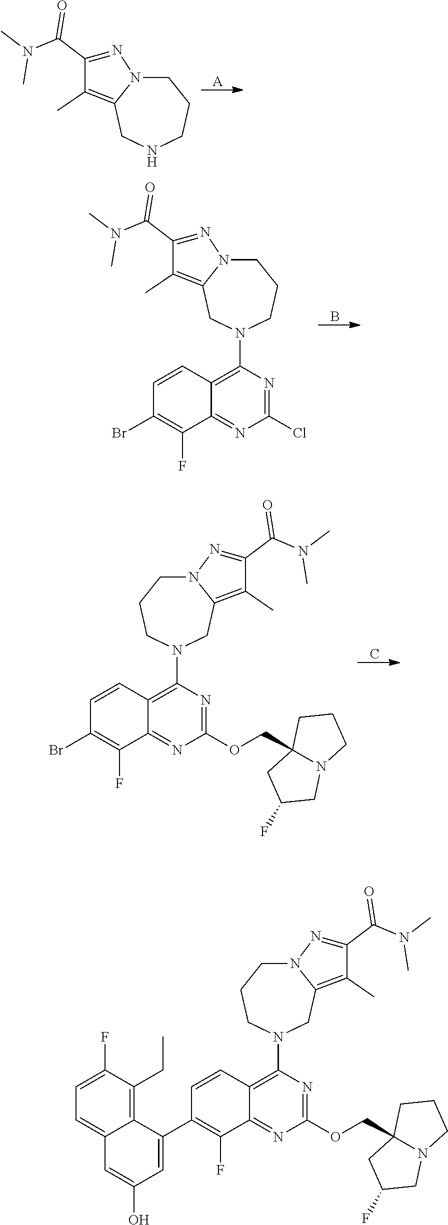

Step A. 5-(7-bromo-2-chloro-8-fluoroquinazolin-4-yl)-N,N,3-trimethyl-5,6,7,8-tetrahydro-4H-pyrazolo[1,5-a][1,4]diazepine-2-carboxamide: To a solution of 7-bromo-2,4-dichloro-8-fluoroquinazoline (840 mg, 1.0 equiv) and DIEA (1.10 g, 3.0 equiv) in dichloromethane (1.0 mL) was added N,N,3-trimethyl-5,6,7,8-tetrahydro-4H-pyrazolo[1,5-a][1,4]diazepine-2-carboxamide (214 mg, 2.0 equiv) at −40° C. The reaction mixture was stirred at −40° C. for 0.5 hours. The reaction mixture was quenched with water (10 mL) and extracted with dichloromethane (3×40 mL). The combined organic layers were dried over anhydrous sodium sulfate, concentrated, and triturated with acetonitrile (15 mL) to afford the title compound (1.10 g, 80% yield) as white solid; LCMS (ESI, M+1): m/z=483.1.

Step B. 5-(7-bromo-8-fluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide: A mixture of 5-(7-bromo-2-chloro-8-fluoroquinazolin-4-yl)-N,N,3-trimethyl-5,6,7,8-tetrahydro-4H-pyrazolo[1,5-a][1,4]diazepine-2-carboxamide (400 mg, 1.0 equiv) and ((2R,7aS)-2-fluorohexahydro-1H-pyrrolizin-7a-yl)methanol (1.18 g, 10 equiv) was stirred at 90° C. for 12 hours. The reaction mixture was slurried with water (15 mL) to afford the title compound (300 mg, 54% yield) as brown solid.

Step C. 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide: To a mixture of 5-(7-bromo-8-fluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide (200 mg, 1.0 equiv) and 5-ethyl-6-fluoro-4-(4,4,5,5-tetramethyl-1,3,2-dioxaborolan-2-yl)naphthalen-2-ol(125 mg, 1.2 equiv) in methoxycyclopentane (2 mL) and water (0.5 mL) were added Ad₂nBup-Pd-G₃ (24.1 mg, 0.1 equiv) and Cs₂CO₃ (323 mg, 3.0 equiv) under N₂ atmosphere. The reaction mixture was stirred at 90° C. for 12 hours under N₂ atmosphere. The mixture was quenched with H₂O (5 mL) and extracted with ethyl acetate (3×15 mL). The combined organic layers were dried over anhydrous sodium sulfate, concentrated and purified with prep-HPLC [Phenomenex luna C18 150×25 mm×10 μm; A: water (FA), B: ACN, B %: 21%-51% over 8 min] to afford the title compound (100 mg, 98% yield, HCOOH salt) as yellow solid; ¹H NMR (400 MHz, METHANOL-d₄) δ=8.56-8.50 (m, 1H), 7.92-7.82 (m, 1H), 7.70-7.58 (m, 1H), 7.38-7.28 (m, 1H), 7.27-7.17 (m, 2H), 6.98-6.88 (m, 1H), 5.49-5.27 (m, 1H), 5.21-5.01 (m, 2H), 4.51-4.42 (m, 2H), 4.41-4.23 (m, 4H), 3.60-3.36 (m, 3H), 3.22-3.15 (m, 1H), 3.15-3.11 (m, 3H), 3.11-3.05 (m, 3H), 2.52-2.27 (m, 6H), 2.25-2.05 (m, 6H), 2.03-1.87 (m, 1H), 0.84-0.67 (m, 3H); LCMS (ESI, M+1): m/z=714.2.

### Example 76

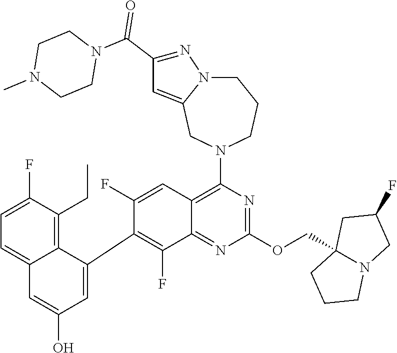 [SMILES: CCc1c(F)ccc2cc(O)cc(-c3c(F)cc4c(N5CCCn6nc(C(=O)N7CCN(C)CC7)cc6C5)nc(OC[C@@]56CCCN5C[C@H](F)C6)nc4c3F)c12]

### (5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone

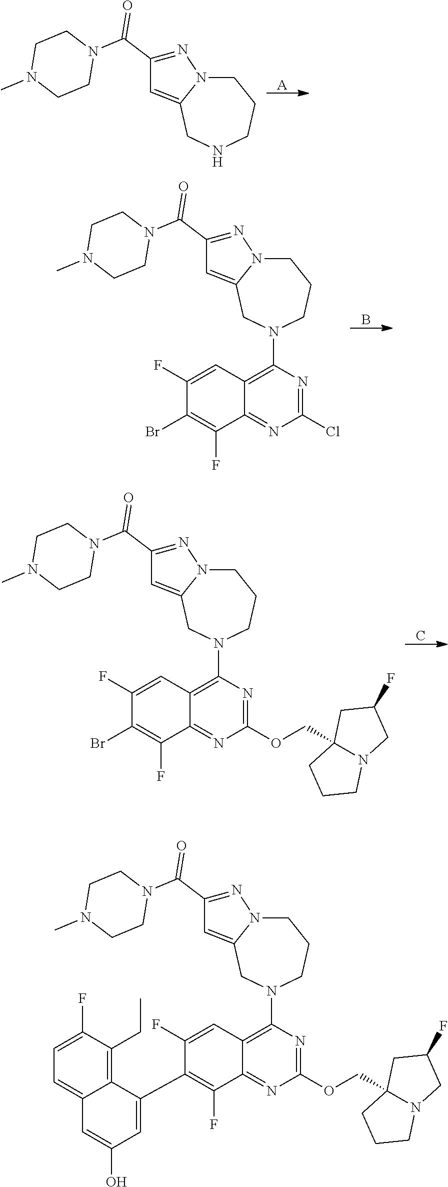

Step A. (5-(7-bromo-2-chloro-6,8-difluoroquinazolin-4-yl)-5,6,7,8-tetrahydro-4H-pyrazolo[1,5-a][1,4]diazepin-2-yl)(4-methylpiperazin-1-yl)methanone: To a solution of 7-bromo-2,4-dichloro-6,8-difluoroquinazoline (880 mg, 1.0 equiv) and DIEA (1.10 g, 3.0 equiv) in dichloromethane (1 mL) was added (4-methylpiperazin-1-yl)(5,6,7,8-tetrahydro-4H-pyrazolo[1,5-a][1,4]diazepin-2-yl)methanone (775 mg, 1.1 equiv). The reaction mixture was stirred at −40° C. for 0.5 hours. The reaction mixture was quenched with water (10 mL) and extracted with dichloromethane (3×40 mL). The combined organic layers were dried over anhydrous sodium sulfate, concentrated and slurried with acetonitrile (15 mL) to afford the title compound (800 mg, 52% yield) as white solid; LCMS (ESI, M+1): m/z=542.2.

Step B. (5-(7-bromo-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone: A mixture of (5-(7-bromo-2-chloro-6,8-difluoroquinazolin-4-yl)-5,6,7,8-tetrahydro-4H-pyrazolo[1,5-a][1,4]diazepin-2-yl)(4-methylpiperazin-1-yl)methanone (400 mg, 1.0 equiv) and ((2R,7aS)-2-fluorohexahydro-1H-pyrrolizin-7a-yl)methanol (1.18 g, 10 equiv) was stirred at 90° C. for 12 hours. The reaction mixture was concentrated and slurried with water (15 mL) to afford the title compound (300 mg, 54% yield) as brown solid; LCMS (ESI, M+1): m/z=665.1.

Step C. (5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone: To a mixture of (5-(7-bromo-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone (250 mg, 1.0 equiv) and 5-ethyl-6-fluoro-4-(4,4,5,5-tetramethyl-1,3,2-dioxaborolan-2-yl)naphthalen-2-ol (143 mg, 1.2 equiv) in methoxycyclopentane (2 mL) and water (0.5 mL) were added Ad₂nBup-Pd-G₃ (41.1 mg, 0.15 equiv) and Cs₂CO₃ (368 mg, 3.0 equiv) under N₂ atmosphere. The reaction mixture was stirred at 90° C. for 12 hours. The reaction mixture was quenched with H₂O (5 mL) and extracted with ethyl acetate (3×15 mL). The combined organic layers were dried over anhydrous sodium sulfate, concentrated and purified with prep-HPLC [Phenomenex luna C18 150×25 mm×10 μm; A: water (FA), B: ACN, B %: 10%-40% over 8 min] to afford the title compound (90.0 mg, 96% yield, HCOOH salt) as yellow solid; ¹H NMR (400 MHz, METHANOL-d₄) δ=8.46-8.43 (m, 1H), 7.84-7.76 (m, 1H), 7.72-7.65 (m, 1H), 7.34-7.29 (m, 1H), 7.29-7.22 (m, 1H), 7.28-7.21 (m, 1H), 7.01-6.96 (m, 1H), 6.76-6.70 (m, 1H), 5.65-5.41 (m, 1H), 5.25-5.06 (m, 2H), 4.59-4.31 (m, 6H), 4.22-4.00 (m, 2H), 3.92-3.64 (m, 5H), 3.40-3.33 (m, 1H), 2.70-2.45 (m, 7H), 2.45-2.31 (m, 7H), 2.30-2.20 (m, 2H), 2.18-2.05 (m, 1H), 0.91-0.72 (m, 3H); LCMS (ESI, M+1): m/z=773.2.

### Example 77

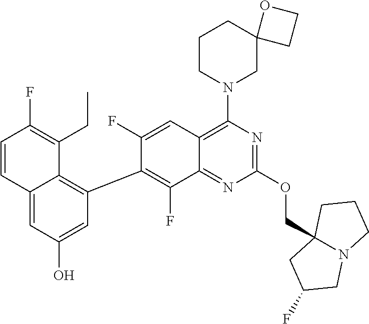 [SMILES: CCc1c(F)ccc2cc(O)cc(-c3c(F)cc4c(N5CCCC6(CCO6)C5)nc(OC[C@@]56CCCN5C[C@H](F)C6)nc4c3F)c12]

### 4-(6,8-difluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-5-ethyl-6-fluoronaphthalen-2-ol

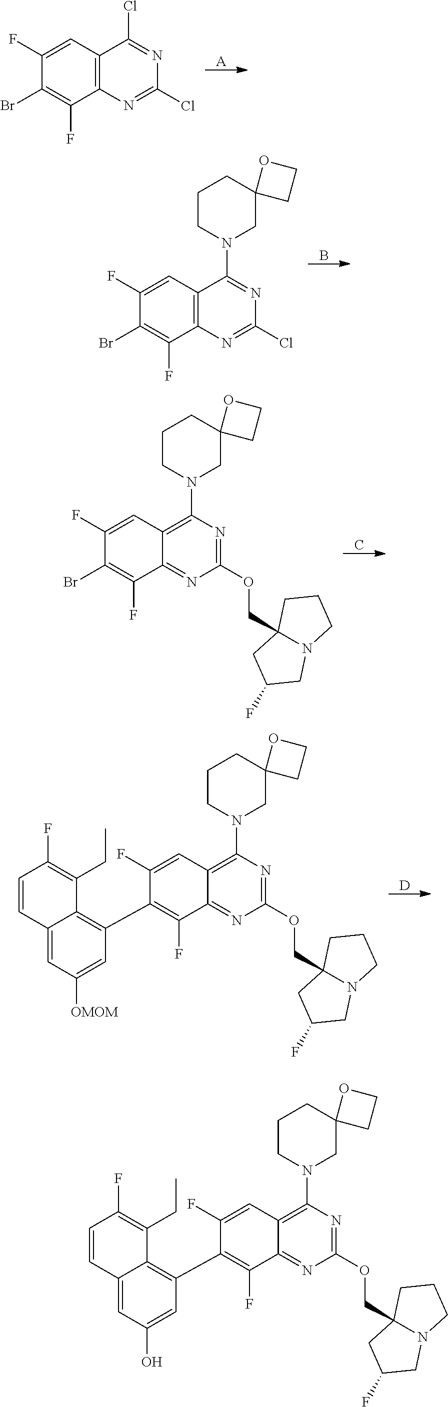

Step A. 6-(7-bromo-2-chloro-6,8-difluoroquinazolin-4-yl)-1-oxa-6-azaspiro[3.5]nonane: To a mixture of 7-bromo-2,4-dichloro-6,8-difluoroquinazoline (1.50 g, 1.0 equiv) and 1-oxa-6-azaspiro[3.5]nonane (905 mg, 1.1 equiv, 0.5 oxalic acid) in DCM (15 mL) were added DIEA (2.47 g, 4.0 equiv) and 4 Å molecular sieve (1.5 g). The reaction mixture was stirred at 0° C. for 15 mins. The mixture was filtered. The filtrate was concentrated and triturated with petroleum ether: ethyl acetate=3:1 (10 V) at 25° C. for 30 mins to afford the title compound (1.50 g, 77.6% yield) as yellow solid. ¹H NMR (400 MHz, DMSO-d₆) δ=8.01 (dd, J=1.6, 9.8 Hz, 1H), 4.39 (t, J=7.8 Hz, 2H), 4.29 (br d, J=13.6 Hz, 1H), 4.12-4.02 (m, 1H), 3.67 (d, J=13.6 Hz, 1H), 3.32 (s, 1H), 2.42-2.28 (m, 2H), 2.15-2.05 (m, 1H), 1.88-1.75 (m, 2H), 1.73-1.61 (m, 1H), 1.32-1.20 (m, 1H).

Step B. 6-(7-bromo-6,8-difluoro-2-(((2R,7aS)-2-fluorohexahydro-1H-pyrrolizin-7a-yl)methoxy)quinazolin-4-yl)-1-oxa-6-azaspiro[3.5]nonane: A mixture of 6-(7-bromo-2-chloro-6,8-difluoroquinazolin-4-yl)-1-oxa-6-azaspiro[3.5]nonane (500 mg, 1.0 equiv) and ((2R,7aS)-2-fluorohexahydro-1H-pyrrolizin-7a-yl)methanol (197 mg, 1.0 equiv) was heated to 110° C. for 2 hours. The reaction mixture was concentrated and triturated with petroleum ether: ethyl acetate=1:5 (3 mL) at 25° C. for 10 mins to afford the title compound (230 mg, 35% yield) as brown solid; LCMS (ESI, M+1): m/z=527.1.

Step C. 6-(7-(8-ethyl-7-fluoro-3-(methoxymethoxy)naphthalen-1-yl)-6,8-difluoro-2-(((2R,7aS)-2-fluorohexahydro-1H-pyrrolizin-7a-yl)methoxy)quinazolin-4-yl)-1-oxa-6-azaspiro[3.5]nonane: To a mixture of 6-(7-bromo-6,8-difluoro-2-(((2R,7aS)-2-fluorohexahydro-1H-pyrrolizin-7a-yl)methoxy)quinazolin-4-yl)-1-oxa-6-azaspiro[3.5]nonane (80.0 mg, 1.0 equiv), 2-(8-ethyl-7-fluoro-3-(methoxymethoxy)naphthalen-1-yl)-4,4,5,5-tetramethyl-1,3,2-dioxaborolane (60.1 mg, 1.1 equiv) in DMF (5 mL) and H₂O (1 mL) were Ad₂nBuP Pd G3(cataCXium® A Pd G3) (11.1 mg, 0.1 equiv) and K₃PO₄ (96.6 mg, 3.0 equiv). The reaction mixture was stirred at 100° C. for 12 hours under N₂ atmosphere. The mixture was diluted with H₂O (10 mL) and extracted with EtOAc (10 mL×3). The combined organic layers were washed with brine (10 mL×3), dried over sodium sulfate, filtered, concentrated and purified by column chromatography [SiO₂, DCM: MeOH=10/1] to afford the title compound (55 mg, crude) as yellow solid; LCMS (ESI, M+1): m/z=681.3.

Step D. 4-(6,8-difluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-5-ethyl-6-fluoronaphthalen-2-ol: To a mixture of 6-(7-(8-ethyl-7-fluoro-3-(methoxymethoxy)naphthalen-1-yl)-6,8-difluoro-2-(((2R,7aS)-2-fluorohexahydro-1H-pyrrolizin-7a-yl)methoxy)quinazolin-4-yl)-1-oxa-6-azaspiro[3.5]nonane (20.0 mg, 1.0 equiv) in DCM (0.5 mL) was added TFA (770 mg, 229.8 equiv). The reaction mixture was stirred at 20° C. for 1 hour. The reaction mixture was quenched by addition of sat. NaHCO₃ aqueous solution (20 mL) at 0° C., and then extracted with EtOAc (20 mL×3). The combined organic layers were washed with brine (20 mL×3), dried over sodium sulfate, filtered, concentrated under reduced pressure and purified with prep-HPLC [column: Phenomenex C18 75*30 mm*3 μm; mobile phase: (water (FA)-CAN); B %: 20%-50%, 7 minutes] to afford the title compound (6.40 mg, 30.8% yield, HCOOH salt) as yellow solid. ¹H NMR (400 MHz, METHANOL-d₄) δ=8.54 (s, 1H), 7.84 (t, J=9.8 Hz, 1H), 7.68 (dd, J=6.0, 9.0 Hz, 1H), 7.29 (d, J=2.4 Hz, 1H), 7.25 (t, J=9.4 Hz, 1H), 7.00 (d, J=2.4 Hz, 1H), 5.41-5.21 (m, 1H), 4.66-4.52 (m, 2H), 4.48-4.36 (m, 1H), 4.35-4.21 (m, 2H), 4.21-4.09 (m, 1H), 3.65 (dd, J=13.8, 19.6 Hz, 1H), 3.36 (br d, J=2.3 Hz, 1H), 3.29-3.15 (m, 3H), 3.08-2.97 (m, 1H), 2.65-2.52 (m, 1H), 2.48 (t, J=7.9 Hz, 2H), 2.44-2.33 (m, 1H), 2.32-2.21 (m, 2H), 2.20-2.11 (m, 1H), 2.09-1.96 (m, 3H), 1.95-1.85 (m, 2H), 1.85-1.74 (m, 1H), 0.86-0.76 (m, 3H); LCMS (ESI, M+1): m/z=637.1.

### Example 78

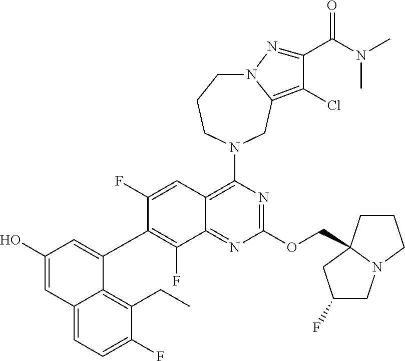 [SMILES: CCc1c(F)ccc2cc(O)cc(-c3c(F)cc4c(N5CCCn6nc(C(=O)N(C)C)c(Cl)c6C5)nc(OC[C@@]56CCCN5C[C@H](F)C6)nc4c3F)c12]

### 3-chloro-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide

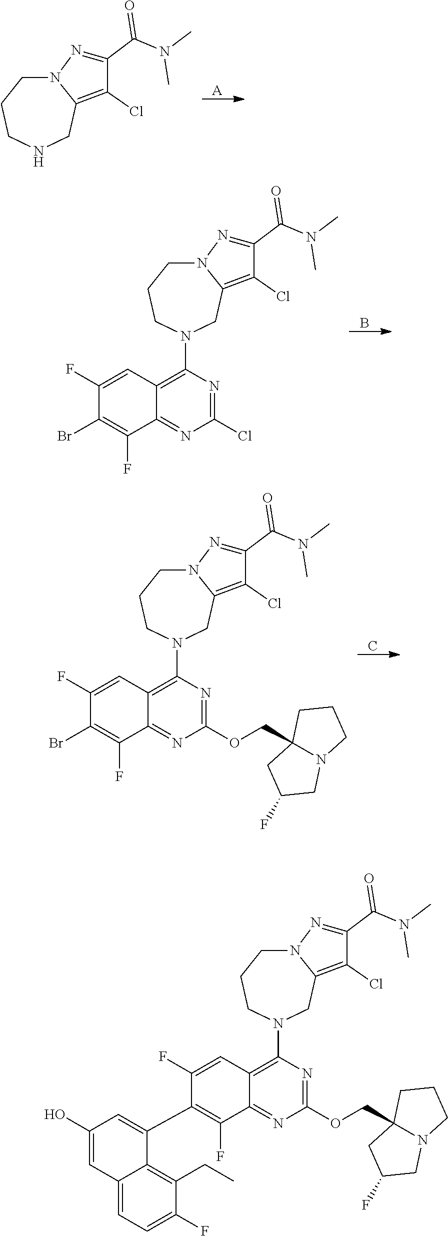

Step A. 5-(7-bromo-2-chloro-6,8-difluoroquinazolin-4-yl)-3-chloro-N,N-dimethyl-5,6,7,8-tetrahydro-4H-pyrazolo[1,5-a][1,4]diazepine-2-carboxamide: To a solution of 7-bromo-2,4-dichloro-6,8-difluoro-quinazoline (1.20 g, 1.0 equiv) in dichloromethane (10 mL) was added N-ethyl-N-propan-2-ylpropan-2-amine (4.78 mL, 5.0 equiv) and 3-chloro-N,N-dimethyl-5,6,7,8-tetrahydro-4H-pyrazolo[1,5-a][1,4]diazepine-2-carboxamide hydrochloride (1.28 g, 1.2 equiv). The reaction mixture was stirred at −40° C. for 1 hour. The mixture was quenched with water (50 ml) and extracted with dichloromethane (3×30 mL). The combined organic layers were washed with brine (20 mL), dried over anhydrous sodium sulfate, concentrated to afford the title compound (2.00 g, crude) as yellow solid.

Step B. 5-(7-bromo-6,8-difluoro-2-(((2R,7aS)-2-fluorohexahydro-1H-pyrrolizin-7a-yl)methoxy)quinazolin-4-yl)-3-chloro-N,N-dimethyl-5,6,7,8-tetrahydro-4H-pyrazolo[1,5-a][1,4]diazepine-2-carboxamide: To a solution of 5-(7-bromo-2-chloro-6,8-difluoro-quinazolin-4-yl)-3-chloro-N,N-dimethyl-4,6,7,8-tetrahydropyrazolo[1,5-a][1,4]diazepine-2-carboxamide (2.00 g, 1.0 equiv) in DMSO (0.5 mL) was added [(2R,8S)-2-fluoro-1,2,3,5,6,7-hexahydropyrrolizin-8-yl]methanol (6.12 g, 10.0 equiv). The mixture was stirred at 90° C. for 48 hours. The mixture diluted with water (5 mL) extracted with dichloromethane (2×20 mL). The combined organic layers were washed with brine (50 mL), dried over anhydrous sodium sulfate, concentrated and purified by reversed-phase flash (0.1% FA condition) to afford the title compound (292 mg, 9% yield) as yellow solid; LCMS (ESI, M+3): m/z=644.3.

Step C. 3-chloro-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N-dimethyl-5,6,7,8-tetrahydro-4H-pyrazolo[1,5-a][1,4]diazepine-2-carboxamide: To a mixture of 5-(7-bromo-6,8-difluoro-2-(((2R,7aS)-2-fluorohexahydro-1H-pyrrolizin-7a-yl)methoxy)quinazolin-4-yl)-3-chloro-N,N-dimethyl-5,6,7,8-tetrahydro-4H-pyrazolo[1,5-a][1,4]diazepine-2-carboxamide (100 mg, 1.0 equiv), 5-ethyl-6-fluoro-4-(4,4,5,5-tetramethyl-1,3,2-dioxaborolan-2-yl)naphthalen-2-ol (73.8 mg, 1.5 equiv) and tripotassium phosphate (1.5 M in water, 0.25 mL, 3.0 equiv) in CPME (1.5 mL) was added CataCXium A Pd G3 (11.3 mg, 0.1 equiv). The reaction mixture was degassed and purged with N₂ for 3 times and stirred at 90° C. for 16 hours under N₂ atmosphere. The mixture was diluted with water (10 mL) and extracted with dichloromethane (2×30 mL). The combined organic layers were washed with brine (30 mL), dried over anhydrous sodium sulfate, concentrated and purified by reversed-phase flash (0.1% FA condition), followed by prep-HPLC [column: Waters Xbridge 150×25 mm×5 μm; A: water (NH₄HCO₃), B:ACN; B %: 49%-79% over 9 min] afford the title compound (7.99 mg, 7% yield) as a yellow solid. ¹H NMR (400 MHz, METHANOL-d₄) δ=7.70-7.63 (m, 2H), 7.30 (d, J=2.4 Hz, 1H), 7.21 (s, 1H), 6.98-6.95 (m, 1H), 5.35 (br s, 1H), 5.12-5.06 (m, 2H), 4.50-4.45 (m, 2H), 4.35-4.26 (m, 2H), 4.24-4.19 (m, 2H), 3.22 (br s, 1H), 3.18 (br d, J=2.8 Hz, 1H), 3.13 (s, 3H), 3.10 (s, 3H), 3.05-2.98 (m, 2H), 2.63-2.51 (m, 1H), 2.48-2.37 (m, 3H), 2.33-2.05 (m, 3H), 2.03-1.86 (m, 3H), 0.84-0.75 (m, 3H); LCMS (ESI, M+1): m/z=752.3.

### Example 79

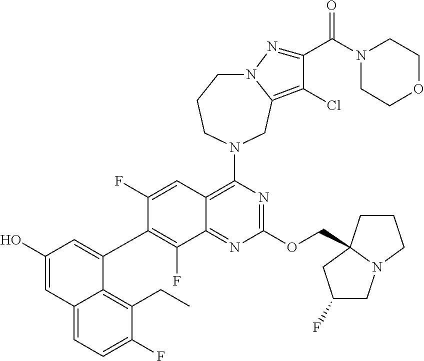 [SMILES: CCc1c(F)ccc2cc(O)cc(-c3c(F)cc4c(N5CCCn6nc(C(=O)N7CCOCC7)c(Cl)c6C5)nc(OC[C@@]56CCCN5C[C@H](F)C6)nc4c3F)c12]

### (3-chloro-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(morpholino)methanone

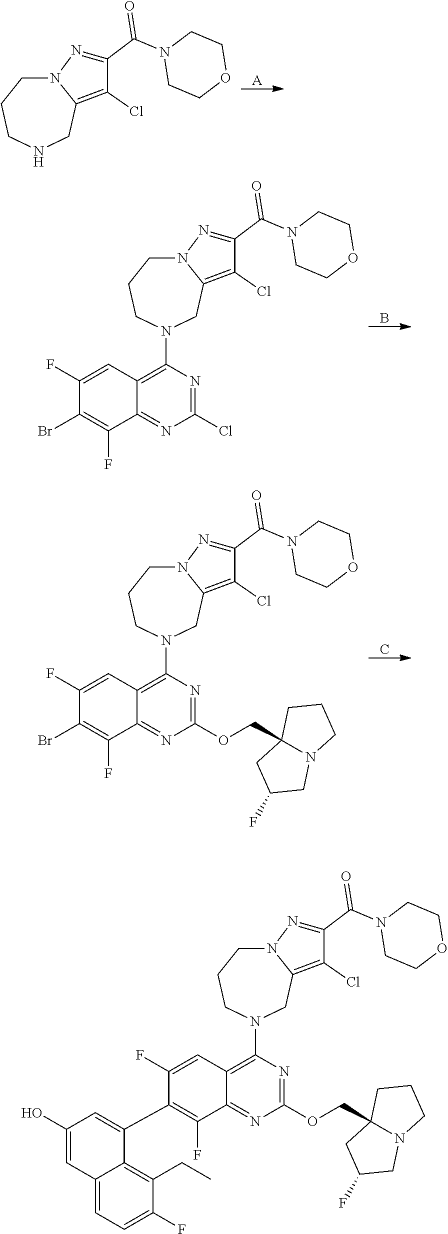

Step A. (5-(7-bromo-2-chloro-6,8-difluoroquinazolin-4-yl)-3-chloro-5,6,7,8-tetrahydro-4H-pyrazolo[1,5-a][1,4]diazepin-2-yl)(morpholino)menthanone: To a solution of (3-chloro-5,6,7,8-tetrahydro-4H-pyrazolo[1,5-a][1,4]diazepin-2-yl)(morpholino)methanone hydrochloride (1.23 g, 1.2 equiv) in DCM (10 mL) was added DIEA (2.06 g, 5.0 equiv) and 7-bromo-2,4-dichloro-6,8-difluoro-quinazoline (1.00 g, 1.0 equiv). The reaction mixture was stirred at −40° C. for 1 hour. The mixture was quenched with H₂O (50 ml) and extracted with DCM (30 mL×3). The combined organic layers were washed with brine (20 mL), dried over anhydrous sodium sulfate, and concentrated to afford the title compound (2.00 g, crude) as yellow oil; LCMS (ESI, M+1): m/z=563.1.

Step B. (5-(7-bromo-6,8-difluoro-2-(((2R,7aS)-2-fluorohexahydro-1H-pyrrolizin-7a-yl)methoxy)guinazolin-4-yl)-3-chloro-5,6,7,8-tetrahydro-4H-pyrazolo[1,5-a][1,4]diazepin-2-yl)(morpholino)methanone: To a solution of [5-(7-bromo-2-chloro-6,8-difluoro-quinazolin-4-yl)-3-chloro-4,6,7,8-tetrahydropyrazolo[1,5-a][1,4]diazepin-2-yl]-morpholino-methanone (2.00 g, 1.0 equiv) in DMSO (2 mL) was added ((2R,7aS)-2-fluorotetrahydro-1H-pyrrolizin-7a(5H)-yl)methanol (5.66 g, 10 equiv). The reaction mixture was stirred at 90° C. for 2 days. The mixture was diluted with water (20 mL) and extracted with DCM (2×100 mL). The combined organic layers were washed with brine (50 mL), dried over anhydrous sodium sulfate, concentrated and purified by reversed-phase flash (0.1% FA condition) to afford the title compound (843 mg, 32% yield) as yellow solid. LCMS (ESI, M+1): m/z=686.2.

Step C. (3-chloro-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(morpholino)methanone: To a mixture of (5-(7-bromo-6,8-difluoro-2-(((2R,7aS)-2-fluorotetrahydro-1H-pyrrolizin-7a(5H)-yl)methoxy)quinazolin-4-yl)-3-chloro-5,6,7,8-tetrahydro-4H-pyrazolo[1,5-a][1,4]diazepin-2-yl)(morpholino)methanone (100 mg, 1.0 equiv), 5-ethyl-6-fluoro-4-(4,4,5,5-tetramethyl-1,3,2-dioxaborolan-2-yl)naphthalen-2-ol (69.2 mg, 1.5 equiv) and K₃PO₄ (1.5 M in water, 292 μL, 3.0 equiv) in CPME (1.5 mL) was added CataCXium A Pd G3 (10.6 mg, 0.1 equiv). The reaction was degassed and purged with nitrogen 3 times and stirred at 90° C. for 5 hours under N₂ atmosphere. The mixture was extracted with DCM (30 mL×2). The combined organic layers were washed with brine (30 mL), dried over anhydrous sodium sulfate, concentrated and purified by reversed-phase flash (0.1% FA condition). The desired fractions were collected and neutralized with solid NaHCO₃ concentrated under vacuum to remove acetonitrile. The aqueous layer was extracted with DCM (3×20 mL). The combined organic layers were washed with brine (30 mL), dried over anhydrous sodium sulfate, concentrated and purified by prep-HPLC (column: Waters Xbridge 150×25 mm×5 μm; A: [water (ammonia hydroxide)]; B: ACN, B %: 46%-76% over 9 min) to afford the title compound (8.35 mg, 7% yield, HOOH salt) as white solid. ¹H NMR (400 MHz, METHANOL-d₄) δ=7.71-7.62 (m, 2H), 7.29 (d, J=2.4 Hz, 1H), 7.25 (t, J=9.2 Hz, 1H), 6.99-6.95 (m, 1H), 5.38-5.25 (m, 1H), 5.14-5.07 (m, 2H), 4.54-4.40 (m, 2H), 4.36-4.27 (m, 2H), 4.25-4.18 (m, 2H), 3.74 (s, 4H), 3.67 (s, 4H), 3.63-3.63 (m, 1H), 3.25-3.14 (m, 2H), 3.07-2.98 (m, 1H), 2.64-2.52 (m, 1H), 2.48-2.39 (m, 3H), 2.34-2.20 (m, 1H), 2.18-2.08 (m, 2H), 2.02-1.95 (m, 2H), 1.94-1.85 (m, 1H), 0.80 (dt, J=2.8, 7.3 Hz, 3H); LCMS (ESI, M+1): m/z=794.5.

### Example 80

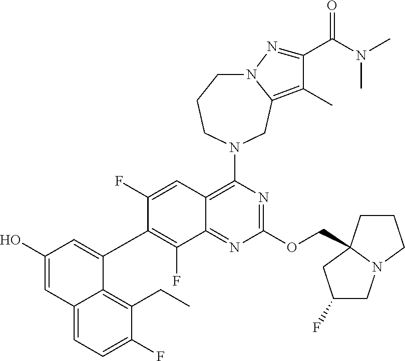 [SMILES: CCc1c(F)ccc2cc(O)cc(-c3c(F)cc4c(N5CCCn6nc(C(=O)N(C)C)c(C)c6C5)nc(OC[C@@]56CCCN5C[C@H](F)C6)nc4c3F)c12]

### 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide

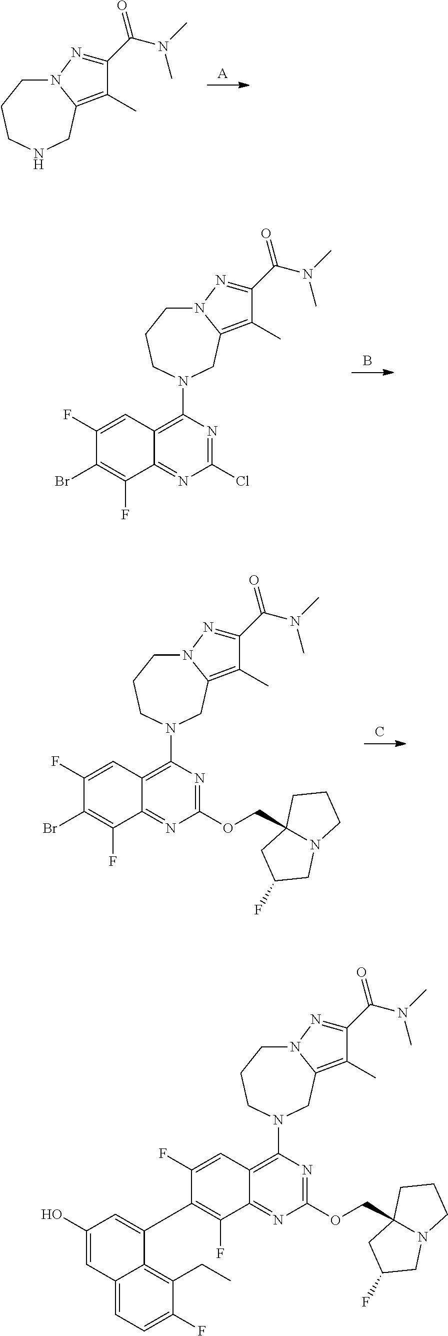

Step A. 5-(7-bromo-2-chloro-6,8-difluoroquinazolin-4-yl)-N,N,3-trimethyl-5,6,7,8-tetrahydro-4H-pyrazolo[1,5-a][1,4]diazepine-2-carboxamide: To a solution of 7-bromo-2,4-dichloro-6,8-difluoroquinazoline (1.0 g, 1.0 equiv) and DIEA (617 mg, 1.5 equiv) in DCM (10 mL) were added a solution of N,N,3-trimethyl-5,6,7,8-tetrahydro-4H-pyrazolo[1,5-a][1,4]diazepine-2-carboxamide (602 mg, 0.85 equiv) and DIEA (823 mg, 2.0 equiv) in DMF (3 mL) at 0° C. The reaction mixture was stirred at 25° C. for 12 hours. The mixture was diluted with water (10 ml) and extracted with ethyl acetate (2×10 mL). The organic layer was washed with brine (10 ml), dried over Na₂SO₄, concentrated purified with reversed phase flash [C18, 0.1% formic acid condition] to afford the title compound (1.2 g, 53% yield, 71% purity) as yellow solid; LCMS (ESI, M+1, M+3, M+5): m/z=499.1, 501.1, 503.1.

Step B. 5-(7-bromo-6,8-difluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide: To a solution of 5-(7-bromo-2-chloro-6,8-difluoroquinazolin-4-yl)-N,N,3-trimethyl-5,6,7,8-tetrahydro-4H-pyrazolo[1,5-a][1,4]diazepine-2-carboxamide (400 mg, 71% purity, 1.0 equiv) in DMSO (2 mL) was added ((2R,7aS)-2-fluorohexahydro-1H-pyrrolizin-7a-yl)methanol (1.27 g, 14 equiv). The mixture was stirred at 90° C. for 24 hours. The mixture was diluted with water (10 ml) and extracted with ethyl acetate (2×10 mL). The organic layer was washed with brine (10 ml), dried over Na₂SO₄, concentrated and purified with reversed phase flash [C18, 0.1% formic acid condition] to afford the title compound (200 mg, 54% yield) as yellow solid; LCMS (ESI, M+1, M+3): m/z=622.2, 624.2.

Step C. 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide: To a solution of 5-(7-bromo-6,8-difluoro-2-(((2R,7aS)-2-fluorohexahydro-1H-pyrrolizin-7a-yl)methoxy)quinazolin-4-yl)-N,N,3-trimethyl-5,6,7,8-tetrahydro-4H-pyrazolo[1,5-a][1,4]diazepine-2-carboxamide (200 mg, 1.0 equiv) and 5-ethyl-6-fluoro-4-(4,4,5,5-tetramethyl-1,3,2-dioxaborolan-2-yl)naphthalen-2-ol (203 mg, 2.0 equiv) in methoxycyclopentane (2 mL) were added K₃PO₄ (1.5 M in H₂O, 642 μL, 3.0 equiv) and CataCXium A Pd G3 (23.4 mg, 0.1 equiv). The mixture was stirred at 90° C. for 5 hours. The mixture was diluted with water (10 ml) and extracted with ethyl acetate (2×10 mL). The organic layer was dried over Na₂SO₄, concentrated and purified with prep-HPLC [column: Phenomenex luna C18 150×25 mm×10 μm; mobile phase: water(FA)−ACN; B %: 18%-48% over 10 minutes] to afford the title compound (101 mg, 42% yield, HCOOH salt) as white solid; ¹H NMR (400 MHz, METHANOL-d₄) δ=7.70-7.63 (m, 2H), 7.29 (d, J=2.6 Hz, 1H), 7.25 (t, J=9.4 Hz, 1H), 6.97 (d, J=2.6 Hz, 1H), 5.50-5.28 (m, 1H), 5.05 (br dd, J=2.8, 8.1 Hz, 2H), 4.47 (br d, J=6.7 Hz, 2H), 4.35 (br d, J=2.6 Hz, 1H), 4.32 (br d, J=11.1 Hz, 3H), 3.57-3.39 (m, 3H), 3.20-3.12 (m, 4H), 3.08 (s, 3H), 2.62-2.44 (m, 2H), 2.43-2.31 (m, 4H), 2.21 (br s, 1H), 2.18 (s, 3H), 2.15-2.05 (m, 2H), 2.03-1.91 (m, 1H), 0.80 (t, J=7.2 Hz, 3H); LCMS (ESI, M+1): m/z=732.4.

### Example 81

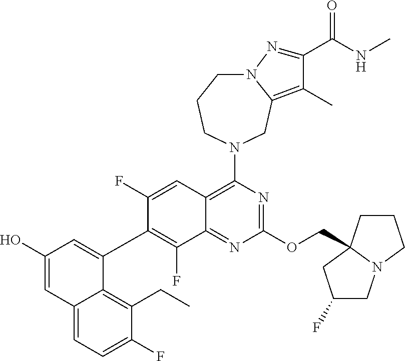 [SMILES: CCc1c(F)ccc2cc(O)cc(-c3c(F)cc4c(N5CCCn6nc(C(=O)NC)c(C)c6C5)nc(OC[C@@]56CCCN5C[C@H](F)C6)nc4c3F)c12]

### 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide

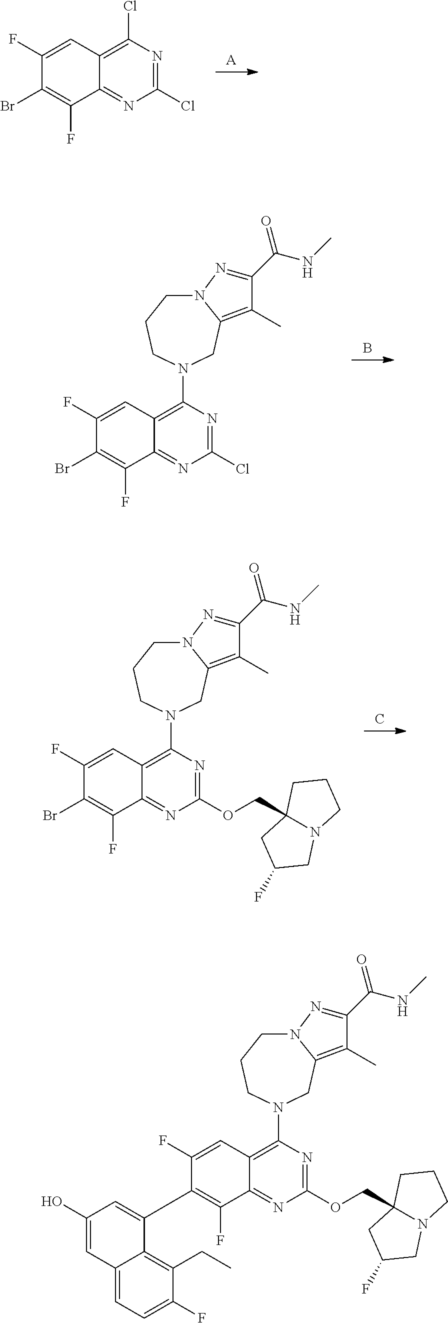

Step A. 5-(7-bromo-2-chloro-6,8-difluoroquinazolin-4-yl)-N,3-dimethyl-5,6,7,8-tetrahydro-4H-pyrazolo[1,5-a][1,4]diazepine-2-carboxamide: To a solution of 7-bromo-2,4-dichloro-6,8-difluoroquinazoline (300 mg, 1.0 equiv) and DIEA (247 mg, 2.0 equiv) in dichloromethane (4.5 mL) was added a solution of N,3-dimethyl-5,6,7,8-tetrahydro-4H-pyrazolo[1,5-a][1,4]diazepine-2-carboxamide (298 mg, 1.5 equiv) in dichloromethane (3 mL) at −40° C. The reaction mixture was stirred at −40° C. for 0.5 hours. The mixture was diluted with water (15 mL) and extracted with ethyl acetate (30 mL). The organic layer was dried over sodium sulfate, concentrated to afford the title compound (447 mg, crude) as yellow solid; LCMS (ESI, M+1, M+3): m/z=485.0, 487.0.

Step B. 5-(7-bromo-6,8-difluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide: To a solution of 5-(7-bromo-2-chloro-6,8-difluoroquinazolin-4-yl)-N,3-dimethyl-5,6,7,8-tetrahydro-4H-pyrazolo[1,5-a][1,4]diazepine-2-carboxamide (250 mg, 1.0 equiv) in DMSO (0.3 mL) was added ((2R,7aS)-2-fluorohexahydro-1H-pyrrolizin-7a-yl)methanol (410 mg, 5.0 equiv). The reaction mixture was stirred at 90° C. for 8 hours. The mixture was diluted with water (10 mL) and extracted with ethyl acetate (20 mL). The organic layer was dried over sodium sulfate, concentrated and purified with reversed-phase flash [C18, 0.1% formic acid condition] to afford the title compound (37.0 mg, 11% yield) as yellow solid; LCMS (ESI, M+1, M+3): m/z=608.0, 610.1.

Step C. 5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide: To a mixture of 5-(7-bromo-6,8-difluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide (37.0 mg, 1.0 equiv), 5-ethyl-6-fluoro-4-(4,4,5,5-tetramethyl-1,3,2-dioxaborolan-2-yl)naphthalen-2-ol (28.8 mg, 1.5 equiv) and K₃PO₄ (1.5 M in water, 122 μL, 3.0 equiv) in methoxycyclopentane (1 mL) was added cataCXium A Pd G3 (4.43 mg, 0.1 equiv). The reaction mixture was degassed and purged with N₂ for 3 times and was stirred at 90° C. for 2 hours under N₂ atmosphere. The mixture was diluted with water (5 mL) and extracted with ethyl acetate (10 mL). The organic layer was dried over sodium sulfate, concentrated and purified with prep-HPLC [Phenomenex luna C18 150×25 mm×10 um; A, water (FA); B, ACN; B %: 17%-47% over 10 mins] to afford the title compound (14.4 mg, 33% yield, HCOOH salt) as white solid; ¹H NMR (400 MHz, METHANOL-d4) δ=7.72-7.62 (m, 2H), 7.32-7.22 (m, 2H), 6.97 (d, J=2.4 Hz, 1H), 5.46-5.25 (m, 1H), 5.11-4.96 (m, 3H), 4.52-4.42 (m, 2H), 4.35-4.22 (m, 4H), 3.51-3.38 (m, 1H), 3.15-3.07 (m, 1H), 2.86 (s, 3H), 2.63-2.48 (m, 1H), 2.46-2.19 (m, 9H), 2.18-2.10 (m, 1H), 2.09-2.01 (m, 2H), 1.98-1.86 (m, 1H), 0.80 (t, J=7.2 Hz, 3H); LCMS (ESI, M+1): m/z=718.5.

### Example 82

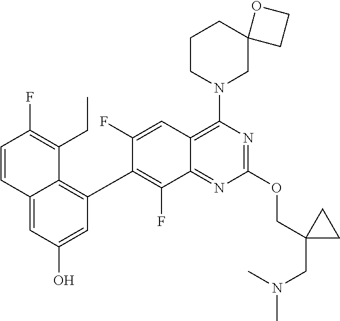 [SMILES: CCc1c(F)ccc2cc(O)cc(-c3c(F)cc4c(N5CCCC6(CCO6)C5)nc(OCC5(CN(C)C)CC5)nc4c3F)c12]

### 4-(2-((1-((dimethylamino)methyl)cyclopropyl)methoxy)-6,8-difluoro-4-(1-oxa-6-azaspiro[3.5]nonan-6-yl)quinazolin-7-yl)-5-ethyl-6-fluoronaphthalen-2-ol

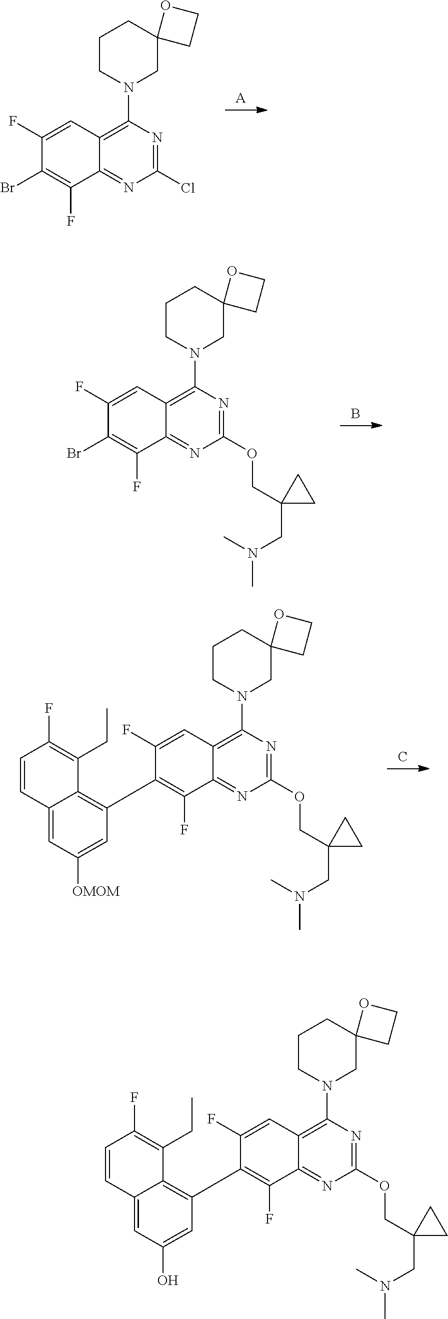

Step A. 1-(1-(((7-bromo-6,8-difluoro-4-(1-oxa-6-azaspiro[3.5]nonan-6-yl)quinazolin-2-yl)oxy)methyl)cyclopropyl)-N,N-dimethylmethanamine: A mixture of 6-(7-bromo-2-chloro-6,8-difluoroquinazolin-4-yl)-1-oxa-6-azaspiro[3.5]nonane (300 mg, 1.0 equiv) and (1-((dimethylamino)methyl)cyclopropyl)methanol (95.8 mg, 1.0 equiv) was heated to 110° C. for 1 hour. The mixture was diluted with H₂O (30 mL) and extracted with EtOAc (30 mL×3). The combined organic layers were washed with brine (30 mL×3), dried over sodium sulfate, filtered, concentrated and purified by reversed-phase MPLC [C18, 0.1% formic acid condition] to afford the title compound (350 mg, 90.2% yield) as a yellow liquid; LCMS (ESI, M+1): m/z=499.0.

Step B. 1-(1-(((7-(8-ethyl-7-fluoro-3-(methoxymethoxy)naphthalen-1-yl)-6,8-difluoro-4-(1-oxa-6-azaspiro[3.5]nonan-6-yl)quinazolin-2-yl)oxy)methyl)cyclopropyl)-N,N-dimethylmethanamine: To a mixture of 1-(1-(((7-bromo-6,8-difluoro-4-(1-oxa-6-azaspiro[3.5]nonan-6-yl)quinazolin-2-yl)oxy)methyl)cyclopropyl)-N,N-dimethylmethanamine (300 mg, 1.0 equiv) and 2-(8-ethyl-7-fluoro-3-(methoxymethoxy)naphthalen-1-yl)-4,4,5,5-tetramethyl-1,3,2-dioxaborolane (239 mg, 1.1 equiv) in DMF (5 mL) and H₂O (1 mL) were added Ad₂nBuP Pd G3 (cataCXium® A Pd G3) (43.9 mg, 0.1 equiv) and K₃PO₄ (384 mg, 3.0 equiv). The reaction was degassed and purged with nitrogen for 3 times, and then stirred at 100° C. for 12 hours. The reaction mixture was diluted with H₂O (30 mL) and extracted with EtOAc (30 mL×3). The combined organic layers were washed with brine (30 mL×3), dried over sodium sulfate, filtered, concentrated and purified by reversed-phase MPLC [C18, 0.1% formic acid condition] to afford the title compound (160 mg, crude) as yellow liquid; LCMS (ESI, M+1): m/z=651.5.

Step C. 4-(2-((1-((dimethylamino)methyl)cyclopropyl)methoxy)-6,8-difluoro-4-(1-oxa-6-azaspiro[3.5]nonan-6-yl)quinazolin-7-yl)-5-ethyl-6-fluoronaphthalen-2-ol: To a mixture of 1-(1-(((7-(8-ethyl-7-fluoro-3-(methoxymethoxy)naphthalen-1-yl)-6,8-difluoro-4-(1-oxa-6-azaspiro[3.5]nonan-6-yl)quinazolin-2-yl)oxy)methyl)cyclopropyl)-N,N-dimethylmethanamine (100 mg, 1.0 equiv) in DCM (3 mL) was added TFA (770 mg, 43.9 equiv). The reaction was stirred at 0° C. for 1 hour. The reaction mixture was quenched with sat. NaHCO₃ aqueous solution (20 mL) at 0° C. and extracted with DCM (20 mL×3). The combined organic layers were washed with brine (20 mL×3), dried over sodium sulfate, filtered, concentrated and purified with prep-HPLC (column: Phenomenex C18 75*30 mm*3 um; mobile phase: [water (FA)−ACN]; B %: 18%-48%, 7 min) to afford the title compound (24.7 mg, 23.8% yield, HCOOH salt) as yellow solid. ¹H NMR (400 MHz, METHANOL-d₄) δ=8.55 (s, 1H), 7.91-7.79 (m, 1H), 7.67 (dd, J=6.0, 8.8 Hz, 1H), 7.29 (d, J=2.4 Hz, 1H), 7.24 (t, J=9.4 Hz, 1H), 7.04-6.88 (m, 1H), 4.62-4.51 (m, 1H), 4.49-4.32 (m, 3H), 4.14 (br t, J=12.8 Hz, 1H), 4.07-3.92 (m, 1H), 3.76-3.60 (m, 1H), 3.37 (br d, J=11.8 Hz, 1H), 2.70 (br d, J=2.4 Hz, 2H), 2.61-2.41 (m, 8H), 2.38-2.20 (m, 2H), 2.16-1.97 (m, 1H), 1.96-1.85 (m, 1H), 1.80 (td, J=4.3, 8.8 Hz, 1H), 0.88-0.75 (m, 5H), 0.61 (br s, 2H); LCMS (ESI, M+1): m/z=607.2.

### Example 83

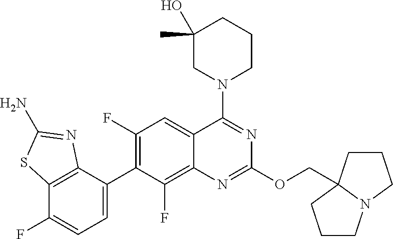 [SMILES: C[C@@]1(O)CCCN(c2nc(OCC34CCCN3CCC4)nc3c(F)c(-c4ccc(F)c5sc(N)nc45)c(F)cc23)C1]

### (3R)-1-(7-(2-amino-7-fluorobenzo[d]thiazol-4-yl)-6,8-difluoro-2-((hexahydro-1H-pyrrolizin-7a-yl)methoxy)quinazolin-4-yl)-3-methylpiperidin-3-ol

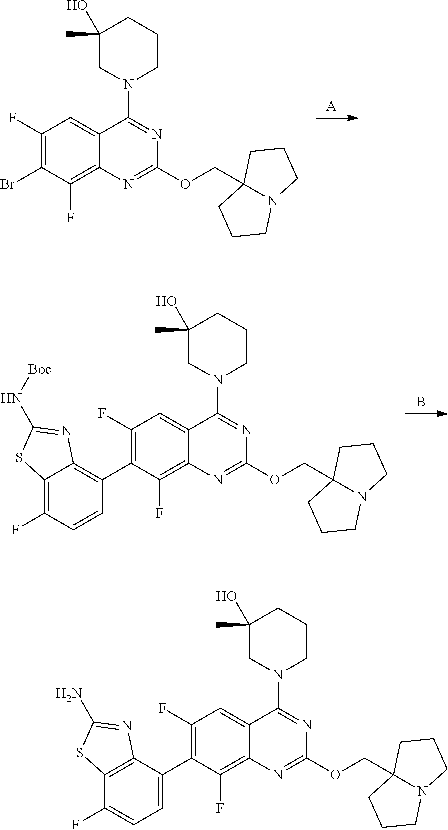

Step A. tert-butyl (4-(6,8-difluoro-2-((hexahydro-1H-pyrrolizin-7a-yl)methoxy)-4-((R)-3-hydroxy-3-methylpiperidin-1-yl)quinazolin-7-yl)-7-fluorobenzo[d]thiazol-2-yl)carbamate: To a solution of (R)-1-(7-bromo-6,8-difluoro-2-((hexahydro-1H-pyrrolizin-7a-yl)methoxy)quinazolin-4-yl)-3-methylpiperidin-3-ol (450 mg, 1.0 equiv), (2-((tert-butoxycarbonyl)amino)-7-fluorobenzo[d]thiazol-4-yl)boronic acid (423 mg, 1.5 equiv) in cyclopentyl methyl ether (6 mL) and were added [2-(2-aminophenyl)phenyl]palladium bis(1-adamantyl)butylphosphane methanesulfonate (65.9 mg, 0.1 equiv), tripotassium phosphate (576.14 mg) and water (1.36 mL). The reaction mixture was degassed and purged with N₂ for 3 times and stirred at 90° C. for 3 hrs. The mixture was diluted with water (5 mL) and extracted with ethyl acetate (3×5 mL). The organic layers were washed with brine (5 mL), dried over anhydrous sodium sulfate (500 mg), concentrated and purified with reversed-phase flash (0.1% FA condition) to afford the title compound (450 mg, 70% yield); LCMS(ESI, M+1): m/z=685.2.

Step B. (3R)-1-(7-(2-amino-7-fluorobenzo[d]thiazol-4-yl)-6,8-difluoro-2-((hexahydro-1H-pyrrolizin-7a-yl)methoxy)quinazolin-4-yl)-3-methylpiperidin-3-ol: To a solution of_tert-butyl (4-(6,8-difluoro-2-((hexahydro-1H-pyrrolizin-7a-yl)methoxy)-4-((R)-3-hydroxy-3-methylpiperidin-1-yl)quinazolin-7-yl)-7-fluorobenzo[d]thiazol-2-yl)carbamate (100 mg, 1.0 equiv) in acetonitrile (1 mL) was added HCl/dioxane (4 M, 2 mL). The mixture was stirred at 0° C. for 1 hr. The reaction mixture was dripped in to ice sodium hydrogen carbonate solution slowly at 0° C. and extracted with ethyl acetate (3×5 mL). The combined organic layers were washed with brine (10 mL), dried over anhydrous sodium sulfate (500 mg), concentrated and purified with prep-HPLC (column: Phenomenex luna C18 150×25 mm×10 μm; A: water(FA); B: ACN; B %: 14%-44%,10 min) and lyophilized to afford the title compound (17.8 mg, 20% yield, HCOOH salt) as white solid; ¹H NMR (400 MHz, METHANOL-d₄) δ=7.73 (ddd, J=1.6, 10.4, 18.4 Hz, 1H), 7.30 (ddd, J=3.6, 5.2, 8.8 Hz, 1H), 6.99 (t, J=8.8 Hz, 1H), 4.42-4.32 (m, 2H), 4.30-4.18 (m, 1H), 4.06 (br d, J=13.0 Hz, 1H), 3.47 (dd, J=5.6, 13.2 Hz, 1H), 3.42-3.34 (m, 1H), 3.29-3.21 (m, 2H), 2.95-2.82 (m, 2H), 2.15 (td, J=6.4, 12.4 Hz, 3H), 2.07-1.92 (m, 4H), 1.90-1.80 (m, 3H), 1.79-1.70 (m, 2H), 1.26 (d, J=2.0 Hz, 3H); LCMS (ESI, M+1): m/z=585.4.

### Example 84

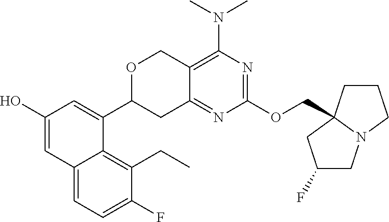 [SMILES: CCc1c(F)ccc2cc(O)cc(C3Cc4nc(OC[C@@]56CCCN5C[C@H](F)C6)nc(N(C)C)c4CO3)c12]

### 4-(4-(dimethylamino)-2-(((2R,7aS)-2-fluorotetrahydro-1H-pyrrolizin-7a(5H)-yl)methoxy)-7,8-dihydro-5H-pyrano[4,3-d]pyrimidin-7-yl)-5-ethyl-6-fluoronaphthalen-2-ol

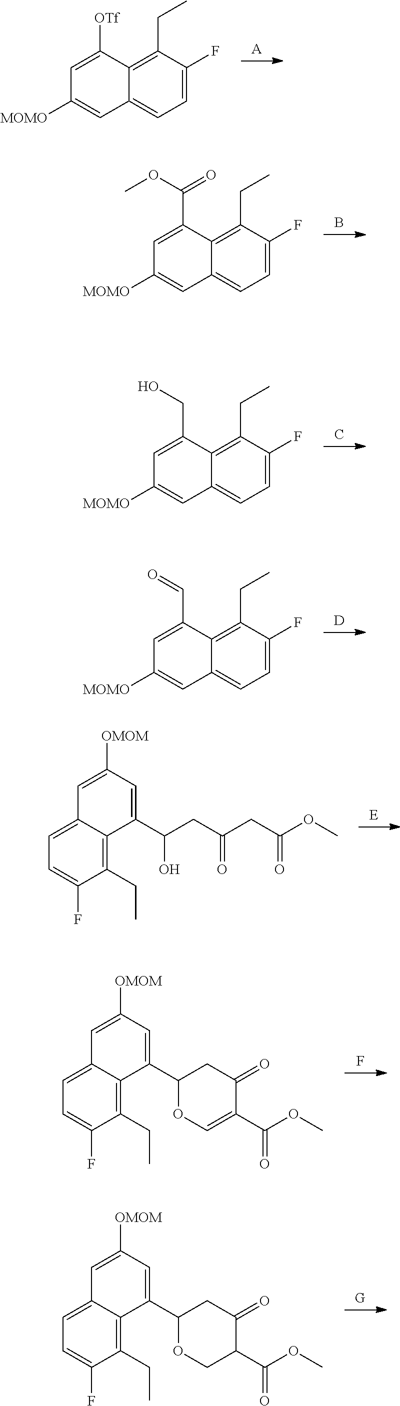

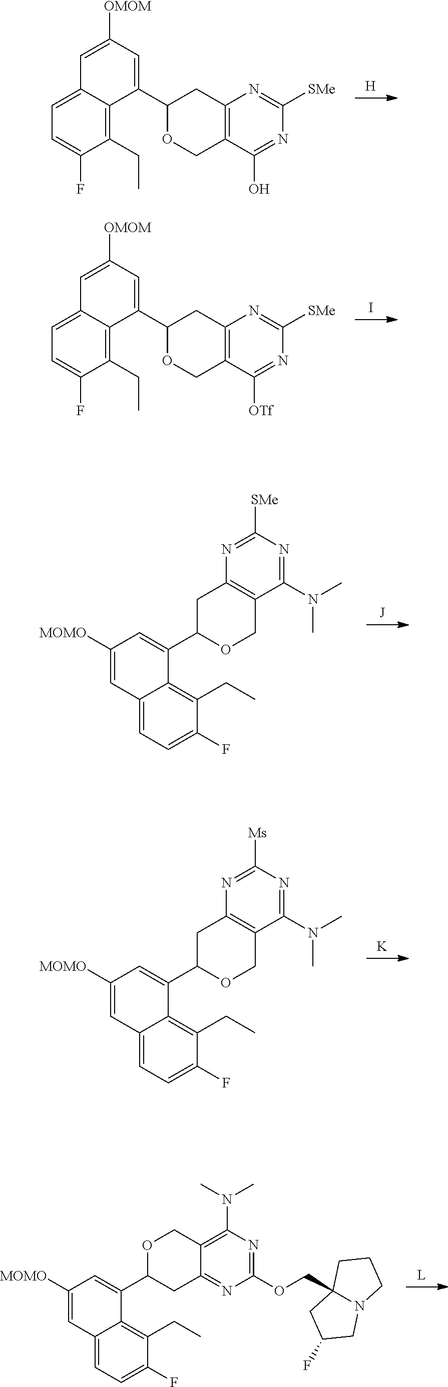

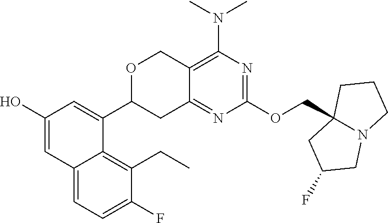 [SMILES: CCc1c(F)ccc2cc(O)cc(C3Cc4nc(OC[C@@]56CCCN5C[C@H](F)C6)nc(N(C)C)c4CO3)c12]

Step A. methyl 8-ethyl-7-fluoro-3-(methoxymethoxy)-1-naphthoate. To a mixture of 8-ethyl-7-fluoro-3-(methoxymethoxy)naphthalen-1-yl trifluoromethanesulfonate (20 g, 52.3 mmol, 1.00 eq), TEA (15.9 g, 157 mmol, 21.8 mL, 3.00 eq) in MeOH (100 mL) and DMSO (100 mL) was added Pd(dppf)Cl₂ (3.83 g, 5.23 mmol, 0.1 eq) in one portion at 20° C. under CO, then heated to 80° C. and stirred for 3 hours. The residue was poured into ice-water (500 mL) and stirred for 30 min. The aqueous phase was extracted with dichloromethane (200 mL×2), the combined organic phase was washed with brine (100 mL), dried with anhydrous Na₂SO₄, filtered and concentrated in vacuum. The residue was purified by a silica gel chromatography column (SiO₂, Petroleum ether/Ethyl acetate=30/1 to 10/1) to give compound to give the title compound (12.0 g, 41.1 mmol, 78.5% yield) as light-yellow solid. ¹H NMR: (400 MHz, CDCl₃) δ 7.53-7.51 (m, 1H), 7.39-7.38 (d, J=4.0 Hz, 1H), 7.26-7.25 (d, J=4.0 Hz, 1H), 7.20-7.15 (m, 1H), 5.19 (s, 2H), 3.90 (s, 3H), 3.43 (s, 3H), 2.81-2.78 (q, 2H), 1.18-1.14 (t, J=8.0 Hz, 3H)

Step B. (8-ethyl-7-fluoro-3-(methoxymethoxy)naphthalen-1-yl)methanol. To a mixture of methyl 8-ethyl-7-fluoro-3-(methoxymethoxy)-1-naphthoate (12.0 g, 41.1 mmol, 1.00 eq) in THF (120 mL) was added LiAlH₄ (1.25 g, 32.8 mmol, 0.80 eq) at 0° C. under N₂. The mixture was stirred at 20° C. for 2 hrs. Add water (1.25 ml) to the reaction solution and stirred for 10 min, add 15% NaOH (1.25 ml) and stirred for 10 min, then add water (4.00 ml) to the reaction solution and stirred for 10 min the mixture was filtered and concentrated in vacuum to give the title compound (9.66 g, crude) as yellow solid.

Step C. 8-ethyl-7-fluoro-3-(methoxymethoxy)-1-naphthaldehyde: To a mixture of (8-ethyl-7-fluoro-3-(methoxymethoxy)naphthalen-1-yl)methanol (9.66 g, 36.6 mmol, 1.00 eq) and MnO₂ (63.6 g, 731 mmol, 20.0 eq) in dichloromethane (100 mL) in one portion at 20° C. under N₂. The mixture was stirred at 20° C. for 16 hrs. The mixture was filtered and concentrated in vacuum to give the title compound (9.00 g, crude) as black brown solid. ¹H NMR: (400 MHz, CDCl₃). δ 10.6 (s, 1H), 7.65-7.64 (d, J=4.0 Hz, 1H), 7.63-7.59 (m, 1H), 7.51-7.50 (d, J=4.0 Hz, 1H), 7.24-7.18 (m, 1H), 5.22 (s, 2H), 3.44 (s, 3H), 2.97-2.95 (q, 2H), 1.33-1.29 (t, J=8.0 Hz, 3H)

Step D. methyl 5-(8-ethyl-7-fluoro-3-(methoxymethoxy)naphthalen-1-yl)-5-hydroxy-3-oxopentanoate: To a mixture of NaH (4.12 g, 103 mmol, 60% purity, 3.00 eq) in THE (50.0 mL) at 20° C. under N₂, was added methyl 3-oxobutanoate (12.0 g, 103 mmol, 11.1 mL, 3.00 eq) at 20° C. The mixture was stirred at 20° C. for 30 min, then was added n-BuLi (2.5 M, 41.2 mL, 3.00 eq) dropwise at −10° C., the mixture was stirred at −10° C. for 30 min, then was added a solution of 8-ethyl-7-fluoro-3-(methoxymethoxy)-1-naphthaldehyde in THE (50.0 mL) drop-wise at −10° C., the mixture was stirred at −10° C. for 2 hrs. The residue was poured into NH₄C1 (200 mL) and stirred for 30 min. The aqueous phase was extracted with ethyl acetate (100 mL×2). The combined organic phase was washed with brine (100 mL), dried with anhydrous Na₂SO₄, filtered and concentrated in vacuum. The residue was purified by column chromatography (SiO₂, Petroleum ether/Ethyl acetate=10/1 to 4/1) to give the title compound (10.0 g, 26.4 mmol, 77.0% yield) as yellow solid. ¹H NMR: EW34545-10-P1A (400 MHz, CDCl₃). δ 7.72-7.71 (d, J=4.0 Hz, 1H), 7.63-7.59 (m, 1H), 7.33-7.34 (d, J=4.0 Hz, 1H), 7.24-7.19 (m, 1H), 6.15-6.12 (m, 1H), 5.31-5.27 (m, 2H), 3.75 (s, 3H), 3.54 (s, 2H), 3.55 (s, 3H), 3.37-3.31 (m, 1H), 3.08-2.95 (m, 3H), 2.80-2.73 (m, 1H), 1.34-1.30 (t, J=8.0 Hz, 3H)

Step E. methyl 2-(8-ethyl-7-fluoro-3-(methoxymethoxy)naphthalen-1-yl)-4-oxo-3,4-dihydro-2H-pyran-5-carboxylate: To a mixture of methyl 5-(8-ethyl-7-fluoro-3-(methoxymethoxy)naphthalen-1-yl)-5-hydroxy-3-oxopentanoate (5.00 g, 13.2 mmol, 1.00 eq) in dichloromethane (50.0 mL) was added DMF-DMA (1.89 g, 15.7 mmol, 2.11 mL, 1.20 eq) in one portion at 20° C. under N₂. The mixture was stirred at 20° C. for 1 hr. Then was added BF₃·Et₂O (2.44 g, 17.2 mmol, 2.12 mL, 1.30 eq) at 20° C., the mixture was stirred at 20° C. for 2 hrs. The residue was poured into ice-water (50.0 mL) and stirred for 30 min. The aqueous phase was extracted with dichloromethane (20.0 mL×2), the combined organic phase was washed with brine (20.0 mL), dried with anhydrous Na₂SO₄, filtered and concentrated in vacuum. The residue was purified by column chromatography (SiO₂, Petroleum ether/Ethyl acetate=10/1 to 5/1) to give the title compound (4.30 g, 11.1 mmol, 83.8% yield) as yellow oil. LCMS: RT=0.935 min, Hz=389.2 [M+H]⁺

Step F. methyl 6-(8-ethyl-7-fluoro-3-(methoxymethoxy)naphthalen-1-yl)-4-oxotetrahydro-2H-pyran-3-carboxylate: To a mixture of methyl 2-(8-ethyl-7-fluoro-3-(methoxymethoxy)naphthalen-1-yl)-4-oxo-3,4-dihydro-2H-pyran-5-carboxylate (3.00 g, 7.72 mmol, 1.00 eq) in THE (30.0 mL) at 20° C. under N₂, then was added L-selectride (1 M, 11.6 mL, 1.50 eq) drop-wise at −70° C. The mixture was stirred at −70° C. for 2 hrs. The residue was poured into NH₄C1 (100 mL) and stirred for 30 min. The aqueous phase was extracted with ethyl acetate (50 mL×2). The combined organic phase was washed with brine (30.0 mL), dried with anhydrous Na₂SO₄, filtered and concentrated in vacuum. The residue was purified by column chromatography (SiO₂, Petroleum ether/Ethyl acetate=10/1) to give the title compound (1.00 g, 2.56 mmol, 33.1% yield) as white solid. ¹H NMR: (400 MHz, CDCl₃), δ 7.56-7.54 (m, 2H), 7.30-7.29 (d, J=4.0 Hz, 1H), 7.14-7.11 (m, 1H), 5.59-5.53 (m, 1H), 5.21 (s, 2H), 5.46-5.45 (m, 1H), 4.42-4.37 (m, 1H), 3.75 (s, 3H), 3.44 (s, 3H), 3.17-3.24 (m, 1H), 2.78-2.71 (m, 1H), 2.64-2.60 (m, 2H), 2.56-2.32 (m, 2H), 1.27-1.25 (m, 3H).

Step G. 7-(8-ethyl-7-fluoro-3-(methoxymethoxy)naphthalen-1-yl)-2-(methylthio)-7,8-dihydro-5H-pyrano[4,3-d]pyrimidin-4-ol: To a mixture of methyl 6-(8-ethyl-7-fluoro-3-(methoxymethoxy)naphthalen-1-yl)-4-oxotetrahydro-2H-pyran-3-carboxylate (1.10 g, 2.82 mmol, 1.00 eq) in MeOH (10.0 mL) was added CH₃₀Na (5 M, 2.82 mL, 5.00 eq) and methyl carbamimidothioate sulfate (784 mg, 2.82 mmol, 1.00 eq) at 0° C. under N₂. The mixture was stirred at 20° C. for 16 hrs. The residue was poured into ice-water (20.0 mL) and stirred for 30 min. The pH was adjusted to around 3 by progressively adding 1M HCl, the aqueous phase was extracted with dichloromethane (20 mL×2). The combined organic phase was washed with brine (10.0 mL), dried with anhydrous Na₂SO₄, filtered and concentrated in vacuum. The residue was purified by column chromatography (SiO₂, Petroleum ether/Ethyl acetate=10/1 to 4/1) to give the title compound (500 mg, 1.16 mmol, 31.7% yield) as white solid. LCMS: EW34545-18-P1A, RT=0.965 min, [M+H]⁺=431.2.

Step H. 7-(8-ethyl-7-fluoro-3-(methoxymethoxy)naphthalen-1-yl)-2-(methylthio)-7,8-dihydro-5H-pyrano[4,3-d]pyrimidin-4-yl trifluoromethanesulfonate: To a mixture of 7-(8-ethyl-7-fluoro-3-(methoxymethoxy)naphthalen-1-yl)-2-(methylthio)-7,8-dihydro-5H-pyrano[4,3-d]pyrimidin-4-ol (500 mg, 1.16 mmol, 1.00 eq) and TEA (353 mg, 3.48 mmol, 3.00 eq) in dichloromethane (5.00 mL) was added Tf₂O (492 mg, 1.74 mmol, 1.50 eq) at −40° C. under N₂. The mixture was stirred at 20° C. for 1 hr. The residue was poured into ice-water (20.0 mL) and stirred for 30 min. The aqueous phase was extracted with dichloromethane (20.0 mL×2), the combined organic phase was washed with brine (10.0 mL), dried with anhydrous Na₂SO₄, filtered and concentrated in vacuum. The residue was purified by column chromatography (SiO₂, Methyl Tertiary Butyl Ether) to give the title compound (650 mg, 924 umol) as yellow oil. LCMS: RT=1.166 min, [M+H]⁺=563.2

Step I. 7-(8-ethyl-7-fluoro-3-(methoxymethoxy)naphthalen-1-yl)-N,N-dimethyl-2-(methylthio)-7,8-dihydro-5H-pyrano[4,3-d]pyrimidin-4-amine: To a mixture of Me₂NH (113 mg, 1.39 mmol, 1.20 eq, HCl) and DIEA (448 mg, 3.47 mmol, 3.00 eq) in THE (6.50 mL) was added 7-(8-ethyl-7-fluoro-3-(methoxymethoxy)naphthalen-1-yl)-2-(methylthio)-7,8-dihydro-5H-pyrano[4,3-d]pyrimidin-4-yl trifluoromethanesulfonate (650 mg, 1.16 mmol, 1.00 eq) in one portion at 20° C. under N₂. The mixture was stirred at 20° C. for 16 hrs. The residue was poured into ice-water (20.0 mL) and stirred for 30 min. The aqueous phase was extracted with dichloromethane (5.00 mL×2), the combined organic phase was washed with brine (5.00 mL), dried with anhydrous Na₂SO₄, filtered and concentrated in vacuum. The residue was purified by column chromatography (SiO₂, Petroleum ether/Ethyl acetate=10/1 to 4/1) to give compound 16 (350 mg, 765 umol, 66.2% yield) as yellow oil. LCMS: RT=0.876 min, [M+H]=458.2.

Step J. 7-(8-ethyl-7-fluoro-3-(methoxymethoxy)naphthalen-1-yl)-N,N-dimethyl-2-(methylsulfonyl)-7,8-dihydro-5H-pyrano[4,3-d]pyrimidin-4-amine: To a mixture of 7-(8-ethyl-7-fluoro-3-(methoxymethoxy)naphthalen-1-yl)-N,N-dimethyl-2-(methylthio)-7,8-dihydro-5H-pyrano[4,3-d]pyrimidin-4-amine (350 mg, 765 umol, 1 eq) and ethyl acetate (5.00 mL) was added m-CPBA (388 mg, 1.91 mmol, 85% purity, 2.50 eq) in one portion at 20° C. under N₂. The mixture was stirred at 20° C. for 1 hr. The residue was poured into Na₂SO₃ (20.0 mL) and stirred for 30 min. The aqueous phase was extracted with dichloromethane (5.00 mL×2), the combined organic phase was washed with brine (5.00 mL), dried with anhydrous Na₂SO₄, filtered and concentrated in vacuum. The residue was purified by column chromatography (SiO₂, Petroleum ether/Ethyl acetate=10/1 to 1/1) to give the title compound (330 mg, 674 umol, 88.1% yield) as white solid.

Step K. 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-N,N-dimethyl-7,8-dihydro-5H-pyrano[4,3-d]pyrimidin-4-amine: To a mixture of ((2R,7aS)-2-fluorotetrahydro-1H-pyrrolizin-7a(5H)-yl)methanol (52.0 mg, 327 umol, 1.60 eq) and t-BuONa (39.3 mg, 409 umol, 2.00 eq) in THE (1.00 mL) was added solution of 7-(8-ethyl-7-fluoro-3-(methoxymethoxy)naphthalen-1-yl)-N,N-dimethyl-2-(methylsulfonyl)-7,8-dihydro-5H-pyrano[4,3-d]pyrimidin-4-amine (100 mg, 204 umol, 1.00 eq) in THE (1.00 mL) drop-wise at 0° C. under N₂. The mixture was stirred at 20° C. for 1 hr. The residue was poured into water (20.0 mL) and stirred for 30 min. The aqueous phase was extracted with dichloromethane (5.00 mL×2), the combined organic phase was washed with brine (5.00 mL), dried with anhydrous Na₂SO₄, filtered and concentrated in vacuum. The residue was purified by prep-TLC (SiO₂, DCM: MeOH=8:1) to give te title compound (70.0 mg, 123 umol, 60.2% yield) as white solid. LCMS: RT=0.824 min, [M+H]⁺=569.4.

Step L. 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-N,N-dimethyl-7,8-dihydro-5H-pyrano[4,3-d]pyrimidin-4-amine: To a mixture of 7-(8-ethyl-7-fluoro-3-(methoxymethoxy)naphthalen-1-yl)-2-(((2R,7aS)-2-fluorotetrahydro-1H-pyrrolizin-7a(5H)-yl)methoxy)-N,N-dimethyl-7,8-dihydro-5H-pyrano[4,3-d]pyrimidin-4-amine (70.0 mg, 123 umol, 1.00 eq) in dioxane (500 uL) was added HCl/dioxane (4 M, 500 uL, 16.3 eq) at 20° C. under N₂, the mixture was stirred at 20° C. for 1 hr. The residue was poured into water (20.0 mL) and stirred for 30 min. The pH was adjusted to around 9 with NaHCO₃ aqueous solution and the mixture was extracted with dichloromethane (5.00 mL×2). The combined organic phase was washed with brine (5.00 mL), dried with anhydrous Na₂SO₄, filtered and concentrated in vacuum to give the title compound (15.0 mg, 28.6 umol, 23.2% yield) as white solid. ¹H NMR: (400 MHz, DMSO-d6). δ 9.82 (s, 1H), 7.44-7.43 (d, J=4.0 Hz, 1H), 7.30-7.26 (t, J=8.0 Hz, 1H), 7.16-7.15 (d, J=4.0 Hz, 1H), 5.49-5.46 (m, 1H), 5.31-5.17 (m, 1H), 5.01-4.98 (d, J=12.0 Hz, 1H), 4.84-4.81 (d, J=12.0 Hz, 1H), 3.98-3.94 (m, 1H), 3.86-3.83 (m, 1H), 3.10-3.04 (m, 3H), 3.02 (s, 6H), 2.98 (s, 1H), 2.83-2.76 (m, 3H), 2.08-1.93 (m, 3H), 1.82-1.71 (m, 3H), 1.27-1.23 (t, J=8.0 Hz, 3H); LCMS: RT=0.791 min, m/z=525.3 (M+H)+.

### Example 85

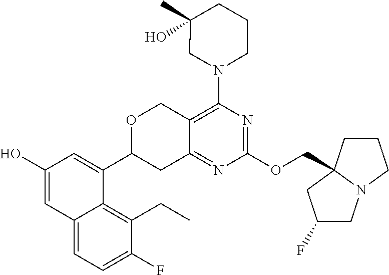 [SMILES: CCc1c(F)ccc2cc(O)cc(C3Cc4nc(OC[C@@]56CCCN5C[C@H](F)C6)nc(N5CCC[C@@](C)(O)C5)c4CO3)c12]

### (3R)-1-(7-(8-ethyl-7-fluoro-3-hydroxynaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-methylpiperidin-3-ol

Synthesized according to example 84. The title compound was obtained as white solid. ¹H NMR: (400 MHz, MeDH-d6). δ 7.63-7.59 (m, 1H), 7.52-7.51 (d, J=4.0 Hz, 1H), 7.24-7.19 (t, J=8.0 Hz, 1H), 7.14 (s, 1H), 5.74-5.64 (m, 1H), 5.36-5.22 (m, 1H), 4.61 (s, 3H), 4.22-4.06 (m, 2H), 3.60-3.51 (m, 1H), 3.48-3.41 (m, 2H), 3.42-3.15 (m, 6H), 3.03-2.99 (m, 1H), 2.98-2.83 (m, 2H), 2.43-2.21 (m, 2H), 2.01-1.95 (m, 4H), 1.94-1.60 (m, 5H), 1.35-1.32 (t, J=8.0 Hz, 3H), 1.25-1.23 (d, J=8.0 Hz, 3H); LCMS: RT=0.799 min, m/z=595.3 (M+H).

### Example 86

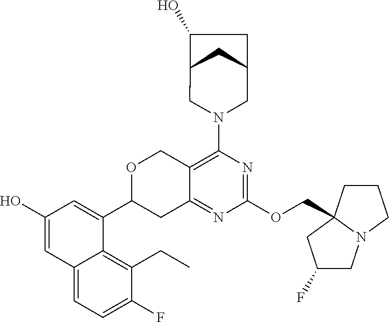 [SMILES: CCc1c(F)ccc2cc(O)cc(C3Cc4nc(OC[C@@]56CCCN5C[C@H](F)C6)nc(N5C[C@@H]6C[C@@H](C5)[C@H](O)C6)c4CO3)c12]

### (1R,5R,6R)-3-(7-(8-ethyl-7-fluoro-3-hydroxynaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-3-azabicyclo[3.2.1]octan-6-ol

Synthesized according to example 84. The title compound was obtained as white solid. ¹H NMR: (400 MHz, MeOD). δ 7.51-7.48 (m, 1H), 7.39-7.42 (dd, J=4.0 Hz, 1H), 7.12-7.07 (t, J=8.0 Hz, 1H), 7.03-7.03 (d, J=4.0 Hz, 1H), 5.57-5.54 (m, 1H), 5.26-5.12 (m, 1H), 5.05-4.93 (m, 2H), 4.49 (s, 3H), 4.36-4.17 (m, 2H), 4.10-3.89 (m, 3H), 3.16-3.02 (m, 4H), 2.98-2.88 (m, 2H), 2.81-2.74 (m, 2H), 2.23-2.00 (m, 6H), 1.98-1.76 (m, 4H), 1.66-1.59 (m, 2H), 1.23-1.20 (t, J=6.0 Hz, 3H); LCMS: EW35153-25-P1H, RT=0.790 min, m/z=607.4 (M+H).

### Example 87

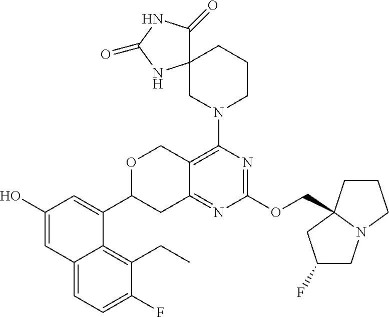 [SMILES: CCc1c(F)ccc2cc(O)cc(C3Cc4nc(OC[C@@]56CCCN5C[C@H](F)C6)nc(N5CCCC6(C5)NC(=O)NC6=O)c4CO3)c12]

### 7-(7-(8-ethyl-7-fluoro-3-hydroxynaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-1,3,7-triazaspiro[4.5]decane-2,4-dione

Synthesized according to example 84. The title compound was obtained as white solid. ¹H NMR: (400 MHz, MeOD): δ 7.52-7.48 (m, 1H), 7.42-7.39 (dd, J=4.0 Hz, 1H), 7.12-7.08 (t, J=8.0 Hz, 1H), 7.03-7.02 (t, J=4.0 Hz, 1H), 5.61-5.57 (m, 1H), 5.27-5.14 (m, 1H), 4.90-4.86 (m, 2H), 4.49 (s, 4H), 4.15-3.96 (m, 3H), 3.69-3.65 (m, 1H), 3.34-3.30 (m, 1H), 3.18-2.92 (m, 5H), 2.85-2.75 (m, 2H), 2.24-2.09 (m, 2H), 2.04-1.97 (m, 2H), 1.90-1.71 (m, 6H), 1.24-1.19 (m, 3H); LCMS: RT=0.804 min, m/z=649.4 (M+H).

### Example 88

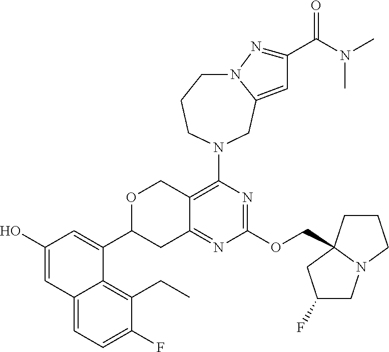 [SMILES: CCc1c(F)ccc2cc(O)cc(C3Cc4nc(OC[C@@]56CCCN5C[C@H](F)C6)nc(N5CCCn6nc(C(=O)N(C)C)cc6C5)c4CO3)c12]

### 5-(7-(8-ethyl-7-fluoro-3-hydroxynaphthalen-1-yl)-2-(((2R,7aS)-2-fluorotetrahydro-1H-pyrrolizin-7a(5H)-yl)methoxy)-7,8-dihydro-5H-pyrano[4,3-d]pyrimidin-4-yl)-N,N-dimethyl-5,6,7,8-tetrahydro-4H-pyrazolo[1,5-a][1,4]diazepine-2-carboxamide

Synthesized according to example 84. The title compound was obtained as white solid. ¹H NMR: (400 MHz, MeOD). δ 7.50-7.46 (m, 1H), 7.36-7.35 (dd, J=4.0 Hz, 1H), 7.11-7.06 (t, J=8.0 Hz, 1H), 7.02-7.01 (d, J=4.0 Hz, 1H), 6.48 (s, 1H), 5.58-5.55 (m, 1H), 5.24-5.10 (m, 1H), 4.94-4.91 (d, J=12.0 Hz, 1H), 4.68-4.66 (m, 3H), 4.40-3.37 (m, 2H), 3.69-3.65 (m, 1H), 4.04-3.89 (m, 3H), 3.81-3.79 (m, 1H), 3.21 (s, 6H), 3.17-3.14 (m, 2H), 3.08-3.02 (m, 2H), 2.97 (s, 2H), 2.91-2.87 (m, 1H), 2.85-2.75 (m, 2H), 2.17-2.05 (m, 3H), 2.01-1.94 (m, 2H), 1.88-1.84 (m, 2H), 1.79-1.74 (m, 2H), 1.23-1.19 (t, J=8.0 Hz, 3H); LCMS: RT=0.825 min, m/z=688.4 (M+H).

### Example 89

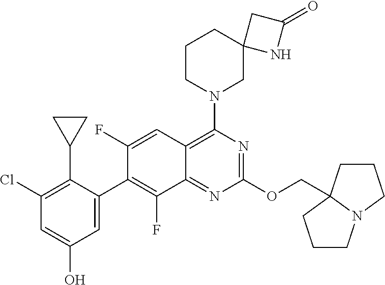 [SMILES: O=C1CC2(CCCN(c3nc(OCC45CCCN4CCC5)nc4c(F)c(-c5cc(O)cc(Cl)c5C5CC5)c(F)cc34)C2)N1]

### 6-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-1,6-diazaspiro[3.5]nonan-2-one

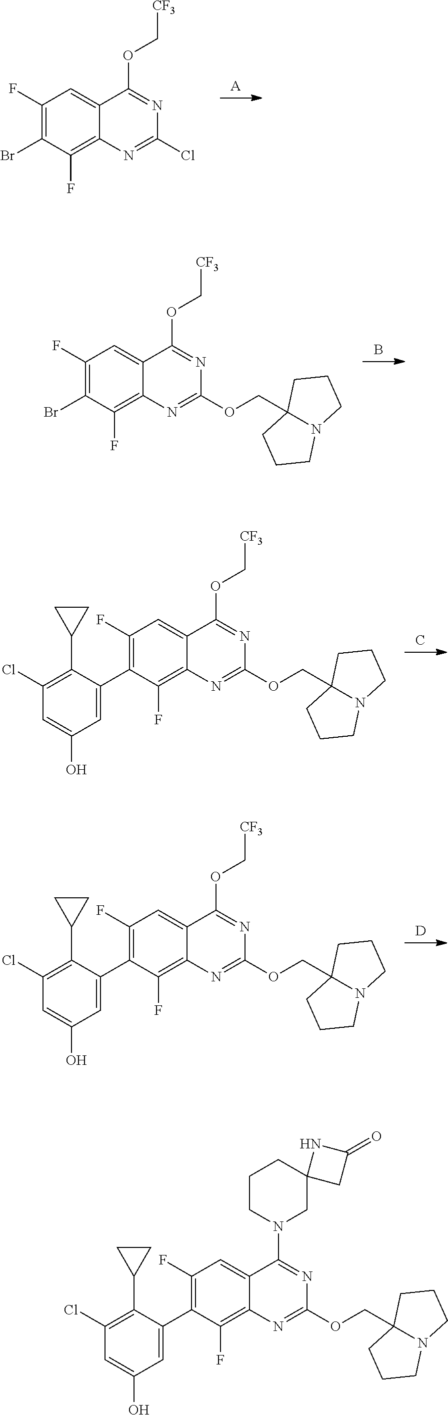

Step A. 7-bromo-6,8-difluoro-2-((tetrahydro-1H-pyrrolizin-7a(5H)-yl)methoxy)-4-(2,2,2-trifluoroethoxy)quinazoline: To a solution of 7-bromo-2-chloro-6,8-difluoro-4-(2,2,2-trifluoroethoxy)quinazoline (3.50 g, 1.0 equiv) in ACN (50 mL) were added Cs₂CO₃ (9.10 g, 3.0 equiv) and 1,2,3,5,6,7-hexahydropyrrolizin-8-ylmethanol (1.3 g, 1.0 equiv). The reaction was stirred at 60° C. for 3 hours. The mixture was diluted with water (40 mL) and extracted with EtOAc (2×30 mL). The combined layers were purified with column chromatography [SiO₂, PE/EtOAc=10/1 to 1/1] to afford the title compound (2.9 g, 58% yield) as white solid; LCMS (ESI, M+1, M+3): m/z=482.0, 484.0.

Step B. 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline: To a mixture of 7-bromo-6,8-difluoro-2-((tetrahydro-1H-pyrrolizin-7a(5H)-yl)methoxy)-4-(2,2,2-trifluoroethoxy)quinazoline (1.40 g, 1.0 equiv), 2-(3-chloro-2-cyclopropyl-5-(methoxymethoxy)phenyl)-4,4,5,5-tetramethyl-1,3,2-dioxaborolane (983 mg, 1.0 equiv) and K₃PO₄ (1.85 g, 3.0 equiv) in THE (30 mL) and H₂O (6.0 mL) was added CataCXium A Pd G3 (211 mg, 0.1 equiv). The reaction was degassed and purged with nitrogen 3 times. The reaction was stirred at 60° C. for 2 hours. The mixture was diluted with water (40 mL) and extracted with EtOAc (2×30 mL). The combined layers were washed with brine (40 mL), dried over sodium sulfate, concentrated and purified with column chromatography [SiO₂, PE/EtOAc=1/1 to 0/1] to afford the title compound (1.10 g, 48% yield) as yellow solid; LCMS (ESI, M+1): m/z=614.1.

Step C. 3-chloro-4-cyclopropyl-5-(6,8-difluoro-2-((tetrahydro-1H-pyrrolizin-7a(5H)-yl)methoxy)-4-(2,2,2-trifluoroethoxy)guinazolin-7-yl)phenol: To a solution of 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline (50.0 mg, 1.0 equiv) in ACN(2.0 mL) was added HCl.dioxane (2 M, 2.0 mL). The reaction was stirred at 0° C. for 1 hour. The mixture was concentrated under vacuum. The residue was diluted with water (0.1 mL). The mixture was adjusted to pH=7 with saturated NaHCO₃(2 mL) and extracted EtOAc (2×2 mL). The combined organic layers were dried over anhydrous sodium sulfate, concentrated to afford the title compound (43.0 mg, 92% yield) as yellow solid; LCMS (ESI, M+1): m/z=570.1.

Step D. 6-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-1,6-diazaspiro[3.5]nonan-2-one: To a solution of 3-chloro-4-cyclopropyl-5-(6,8-difluoro-2-((tetrahydro-1H-pyrrolizin-7a(5H)-yl)methoxy)-4-(2,2,2-trifluoroethoxy)quinazolin-7-yl)phenol (33.0 mg, 1.0 equiv) in DMF (2.0 mL) were added K₃PO₄ (24.5 mg, 2.0 equiv) and 1,8-diazaspiro[3.5]nonan-2-one (20.2 mg, 2.5 equiv). The reaction was stirred at 100° C. for 12 hours. The mixture was filtered and purified with prep-HPLC [Waters Xbridge 150×25 mm×5 um; mobile phase: water (ammonia hydroxide v/v)−ACN; B %: 22%-52%, 9 min] to afford the title compound (8.0 mg, 22% yield) as white solid; ¹H NMR (400 MHz, DMSO-d₆) δ=7.60 (d, J=9.2 Hz, 1H), 6.95 (d, J=2.4 Hz, 1H), 6.63 (d, J=2.4 Hz, 1H), 4.14 (d, J=12.4 Hz, 1H), 3.99 (d, J=12.0 Hz, 1H), 3.84-3.68 (m, 1H), 2.70-2.53 (m, 2H), 2.44-2.24 (m, 6H), 2.06-1.90 (m, 3H), 1.75-1.41 (m, 11H), 0.71-0.51 (m, 2H), 0.11 (br d, J=4.4 Hz, 2H); LCMS (ESI, M+1): m/z=610.1.

### Example 90

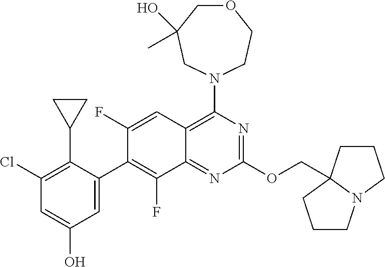 [SMILES: CC1(O)COCCN(c2nc(OCC34CCCN3CCC4)nc3c(F)c(-c4cc(O)cc(Cl)c4C4CC4)c(F)cc23)C1]

### 4-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-6-methyl-1,4-oxazepan-6-ol

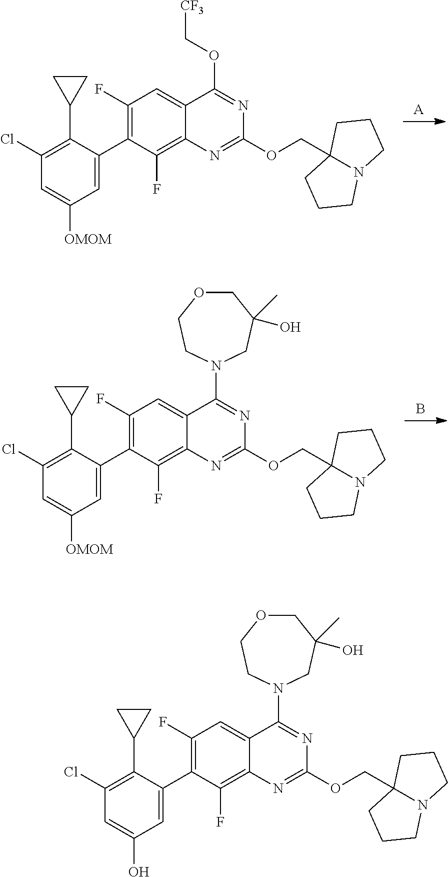

Step A. 4-(7-(3-chloro-2-cyclopropyl-5-(methoxymethoxy)phenyl)-6,8-difluoro-2-((tetrahydro-1H-pyrrolizin-7a(5H)-yl)methoxy)quinazolin-4-yl)-6-methyl-1,4-oxazepan-6-ol: To a mixture of 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline (60.0 mg, 1.0 equiv) and 6-methyl-1,4-oxazepan-6-ol (25.6 mg, 2.0 equiv) in DMF (1.0 mL) were added 4 Å molecular sieve (10.0 mg, 1.0 equiv) and K₃PO₄ (31.1 mg, 1.5 equiv). The reaction was stirred at 60° C. for 12 hours. The mixture was filtered and purified with prep-TLC [SiO₂, DCM/MeOH=10/1] to afford the title compound (35.4 mg, 5500 yield) as yellow solid. LCMS (ESI, M+1): m/z=645.2.

Step B. 4-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-6-methyl-1,4-oxazepan-6-ol: To a solution of 4-(7-(3-chloro-2-cyclopropyl-5-(methoxymethoxy)phenyl)-6,8-difluoro-2-((tetrahydro-1H-pyrrolizin-7a(5H)-yl)methoxy)quinazolin-4-yl)-6-methyl-1,4-oxazepan-6-ol (35.4 mg, 1.0 equiv) in MeCN (1.0 mL) was added HCl.dioxane (4 M, 1.0 mL). The reaction was stirred at 0° C. for 0.5 hours. The mixture was concentrated under vacuum. The residue was diluted with water (0.5 mL). The mixture was adjusted to pH=7 with saturated NaHCO₃(1 mL) and extracted with EtOAc (2×2 mL). The organic layers were dried over anhydrous sodium sulfate, concentrated and purified with prep-HPLC [Waters Xbridge 150×25 mm×5 um; mobile phase: water (ammonia hydroxide v/v)−ACN; B %: 43%-73%, 9 min] to afford the title compound (2.80 mg, 9.0% yield) as white solid; ¹H NMR (400 MHz, METHANOL-d₄) δ=7.63 (br d, J=9.2 Hz, 1H), 6.94 (d, J=2.4 Hz, 1H), 6.63 (dd, J=2.4, 6.0 Hz, 1H), 4.68-4.54 (m, 2H), 4.24-4.09 (m, 2H), 3.93-3.77 (m, 2H), 3.70-3.56 (m, 2H), 3.50 (d, J=12.4 Hz, 1H), 3.47-3.39 (m, 1H), 2.87-2.77 (m, 1H), 2.72-2.56 (m, 5H), 2.19-2.06 (m, 3H), 1.82-1.67 (m, 4H), 1.66-1.55 (m, 2H), 1.10 (s, 3H), 0.62 (br t, J=6.8 Hz, 2H), 0.17 (br s, 2H); LCMS (ESI, M+1): m/z=601.3.

### Example 91

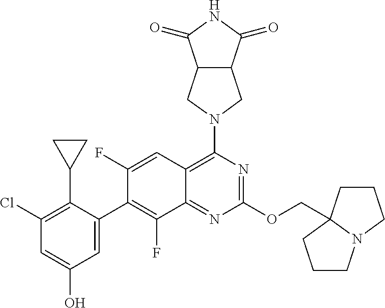 [SMILES: O=C1NC(=O)C2CN(c3nc(OCC45CCCN4CCC5)nc4c(F)c(-c5cc(O)cc(Cl)c5C5CC5)c(F)cc34)CC12]

### 5-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)tetrahydropyrrolo[3,4-c]pyrrole-1,3(2H,3aH)-dione

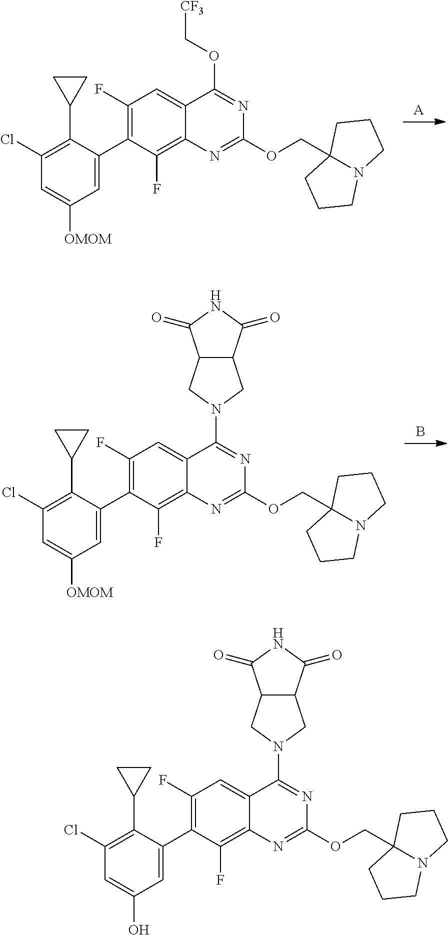

Step A. 5-(7-(3-chloro-2-cyclopropyl-5-(methoxymethoxy)phenyl)-6,8-difluoro-2-((tetrahydro-1H-pyrrolizin-7a(5H)-yl)methoxy)guinazolin-4-yl)tetrahydropyrrolo[3,4-c]pyrrole-1,3(2H,3aH)-dione: To a mixture of 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline (60.0 mg, 1.0 equiv) and tetrahydropyrrolo[3,4-c]pyrrole-1,3(2H,3aH)-dione (50.0 mg, 3.2 equiv) in DMF (1.0 mL) were added 4 Å molecular sieve (10.0 mg) and K₃PO₄ (31.1 mg, 1.5 equiv). The reaction was stirred at 60° C. for 12 hours. The mixture was filtered and purified with prep-TLC [SiO₂, DCM/MeOH=10/1] to afford the title compound (30.2 mg, 43% yield) as yellow solid. LCMS (ESI, M+1): m/z=654.2.

Step B. 5-(7-(3-chloro-2-cyclopropyl-5-hydroxyphenyl)-6,8-difluoro-2-((tetrahydro-1H-pyrrolizin-7a(5H)-yl)methoxy)guinazolin-4-yl)tetrahydropyrrolo[3,4-c]pyrrole-1,3(2H,3aH)-dione: To a solution of 5-(7-(3-chloro-2-cyclopropyl-5-(methoxymethoxy)phenyl)-6,8-difluoro-2-((tetrahydro-1H-pyrrolizin-7a(5H)-yl)methoxy)quinazolin-4-yl)tetrahydropyrrolo[3,4-c]pyrrole-1,3(2H,3aH)-dione (30.2 mg, 1.0 equiv) in MeCN (1.0 mL) was added HCl.dioxane (4M, 1.0 mL). The reaction was stirred at 0° C. for 0.5 hours. The mixture was concentrated under vacuum. The mixture was diluted with NaHCO₃(2 mL) and extracted with EtOAc (2×2 mL). The organic layers were dried over anhydrous sodium sulfate, concentrated and purified with prep-HPLC [Phenomenex C18 75×30 mm×3 um; mobile phase: water(FA)−ACN;B %: 18%-48%,7 min; B %: 43%-73%, 9 min] to afford the title compound (5.0 mg, 15.2% yield, 0.2 HCOOH) as white solid; ¹H NMR (400 MHz, METHANOL-d₄) δ=7.62 (dd, J=1.2, 8.8 Hz, 1H), 6.94 (d, J=2.4 Hz, 1H), 6.67-6.59 (m, 1H), 4.19-4.06 (m, 2H), 3.85 (qd, J=7.6, 11.2 Hz, 1H), 3.54 (br t, J=6.4 Hz, 2H), 3.51-3.45 (m, 4H), 3.21 (br dd, J=7.2, 11.2 Hz, 2H), 2.22-2.01 (m, 3H), 1.84-1.63 (m, 6H), 1.37-1.26 (m, 1H), 0.61 (br t, J=6.8 Hz, 2H), 0.16 (br d, J=4.4 Hz, 2H); LCMS (ESI, M+1): m/z=610.3.

### Example 92

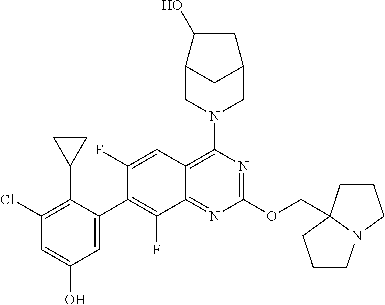 [SMILES: Oc1cc(Cl)c(C2CC2)c(-c2c(F)cc3c(N4CC5CC(O)C(C5)C4)nc(OCC45CCCN4CCC5)nc3c2F)c1]

### 3-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-3-azabicyclo[3.2.1]octan-6-ol

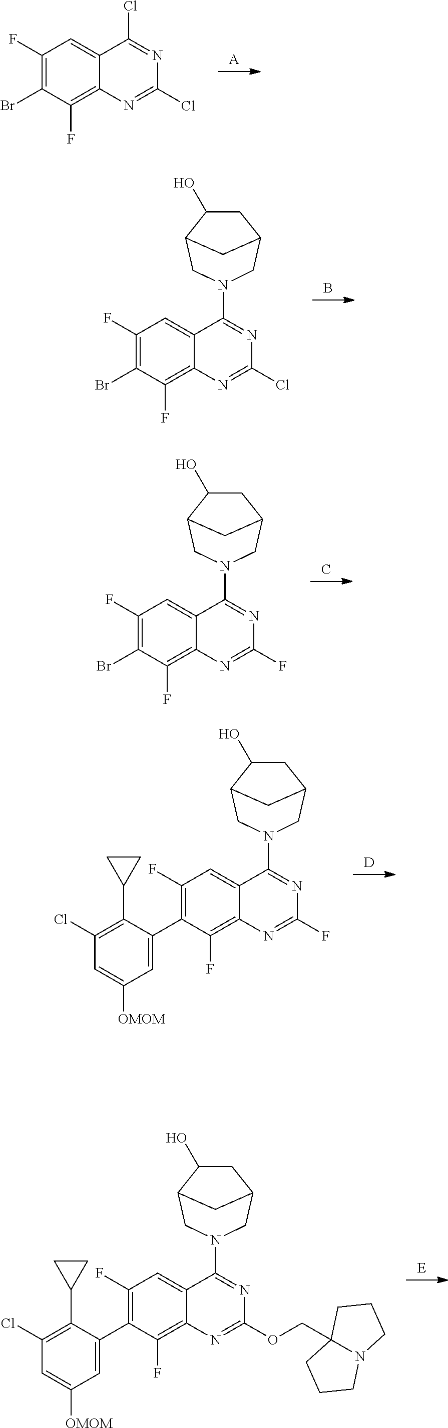

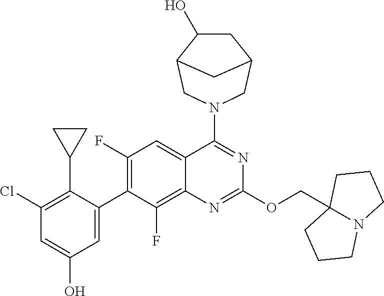 [SMILES: Oc1cc(Cl)c(C2CC2)c(-c2c(F)cc3c(N4CC5CC(O)C(C5)C4)nc(OCC45CCCN4CCC5)nc3c2F)c1]

Step A. 3-(7-bromo-2-chloro-6,8-difluoroquinazolin-4-yl)-3-azabicyclo[3.2.1]octan-6-ol: To a mixture of 7-bromo-2,4-dichloro-6,8-difluoroquinazoline (800 mg, 1.2 equiv) and 4 Å molecular sieve (1.50 g) in DCM (15 mL) were added DIEA (1.54 g, 3.0 equiv) and 3-azabicyclo[3.2.1]octan-6-ol (320 mg, 1.0 equiv). The reaction was stirred at 0° C. for 0.5 hours. The mixture was diluted with ice-water (50 mL) and extracted with EtOAc (3×50 mL). The combined organic layers were washed with brine (50 mL), dried over anhydrous sodium sulfate, concentrated and purified with re-crystallization from EtOAc (20 mL) at 25° C. to afford the title compound (600 mg, 68% yield) as yellow solid; LCMS (ESI, M+1, M+3): m/z=404.1, 406.1.

Step B. 3-(7-bromo-2,6,8-trifluoroquinazolin-4-yl)-3-azabicyclo[3.2.1]octan-6-ol: To a solution of 3-(7-bromo-2-chloro-6,8-difluoro-quinazolin-4-yl)-3-azabicyclo[3.2.1]octan-6-ol (600 mg, 1.0 equiv) in DMSO (5.0 mL) was added KF (1.77 g, 21 equiv). The reaction was stirred at 120° C. for 12 hours. The mixture was filtered. The filtrate was diluted with EtOAc (50 mL). The mixture was washed with brine (2×30 mL), dried over anhydrous sodium sulfate, concentrated to afford the title compound (570 mg, crude) as yellow solid; LCMS (ESI, M+1, M+3): m/z=387.9, 389.9

Step C. 3-(7-(3-chloro-2-cyclopropyl-5-(methoxymethoxy)phenyl)-2,6,8-trifluoroquinazolin-4-yl)-3-azabicyclo[3.2.1]octan-6-ol: To a mixture of 3-(7-bromo-2,6,8-trifluoroquinazolin-4-yl)-3-azabicyclo[3.2.1]octan-6-ol (570 mg, 1.0 equiv) and 2-[3-chloro-2-cyclopropyl-5-(methoxymethoxy)phenyl]-4,4,5,5-tetramethyl-1,3,2-dioxaborolane (746 mg, 1.5 equiv) in THE (15 mL) and H₂O (3.0 mL) were added CataCXium A Pd G3 (214 mg, 0.2 equiv) and K₃PO₄ (1.5 M, 2.94 mL, 3 equiv). The reaction was stirred at 60° C. for 2.5 hours. The mixture was quenched with saturated NaHCO₃ aqueous solution (50 mL) and extracted with EtOAc (3×30 mL). The combined organic layers were washed with brine (30 mL), dried over anhydrous sodium sulfate, concentrated and purified with column chromatography [SiO₂, PE/EtOAc=10/1 to 0/1] to afford the title compound (600 mg, 79% yield) as yellow solid; LCMS (ESI, M+1): m/z=520.2.

Step D. 3-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-3-azabicyclo[3.2.1]octan-6-ol: To a solution of (tetrahydro-1H-pyrrolizin-7a(5H)-yl)methanol (32.6 mg, 1.2 equiv) in THE (1.0 mL) was added NaH (15.4 mg, 60% purity, 2.0 equiv) at 0° C., and then 3-[7-[3-chloro-2-cyclopropyl-5-(methoxymethoxy)phenyl]-2,6,8-trifluoro-quinazolin-4-yl]-3-azabicyclo[3.2.1]octan-6-ol (100 mg, 1.0 equiv) was added into the mixture. The reaction was stirred at 20° C. for 1 hour. The mixture was quenched with water (10.0 mL) and extracted with EtOAc (3×10 mL). The combined organic layers were washed with brine (10 mL), dried over anhydrous sodium sulfate, filtered, concentrated and purified with prep-HPLC [column: Phenomenex luna C18 150×25 mm×10 um; mobile phase: water (FA)−ACN; B %: 22%-52%,10 minutes] to afford the title compound (15.0 mg, 10% yield) as yellow solid; LCMS (ESI, M+1): m/z=641.4.

Step E. 3-(7-(3-chloro-2-cyclopropyl-5-hydroxyphenyl)-6,8-difluoro-2-((hexahydro-1H-pyrrolizin-7a-yl)methoxy)quinazolin-4-yl)-3-azabicyclo[3.2.1]octan-6-ol: To a solution of 3-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-3-azabicyclo[3.2.1]octan-6-ol (15.0 mg, 1.0 equiv) in MeOH (1.0 mL) was added HCl.MeOH (4 M, 1.0 mL). The reaction was stirred at 20° C. for 0.5 hours. The mixture was concentrated under vacuum. The residue was diluted with water (0.5 mL). The mixture was adjusted to pH=7 with saturated NaHCO₃(10 mL) and extracted with EtOAc (2×10 mL). The combined organic layers were dried over anhydrous sodium sulfate, concentrated and purified with prep-HPLC [column: Phenomenex luna C18 150×25 mm×10 um; mobile phase: [water (FA)−ACN]; B %: 20%-50%, 10 minutes] to afford the title compound (8.00 mg, 53% yield, 0.79 HCOOH) as white solid; ¹H NMR (400 MHz, DMSO-d6) δ=7.63 (br d, J=10.4 Hz, 1H), 6.97 (d, J=2.8 Hz, 1H), 6.68 (s, 1H), 4.50-4.34 (m, 1H), 4.15 (br s, 1H), 4.08 (s, 3H), 3.45-3.36 (m, 1H), 3.33-3.24 (m, 1H), 3.08-2.96 (m, 2H), 2.64 (td, J=6.8, 10.0 Hz, 2H), 2.44-2.40 (m, 1H), 2.11 (br s, 1H), 1.98-1.74 (m, 8H), 1.67-1.55 (m, 4H), 1.42 (br dd, J=6.8, 13.6 Hz, 1H), 0.63-0.48 (m, 2H), 0.08 (br d, J=5.6 Hz, 2H); LCMS (ESI, M+1): m/z=597.2.

### Example 93

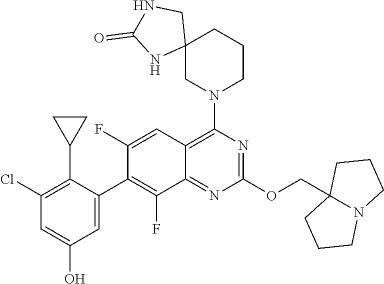 [SMILES: O=C1NCC2(CCCN(c3nc(OCC45CCCN4CCC5)nc4c(F)c(-c5cc(O)cc(Cl)c5C5CC5)c(F)cc34)C2)N1]

### 7-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-1,3,7-triazaspiro[4.5]decan-2-one

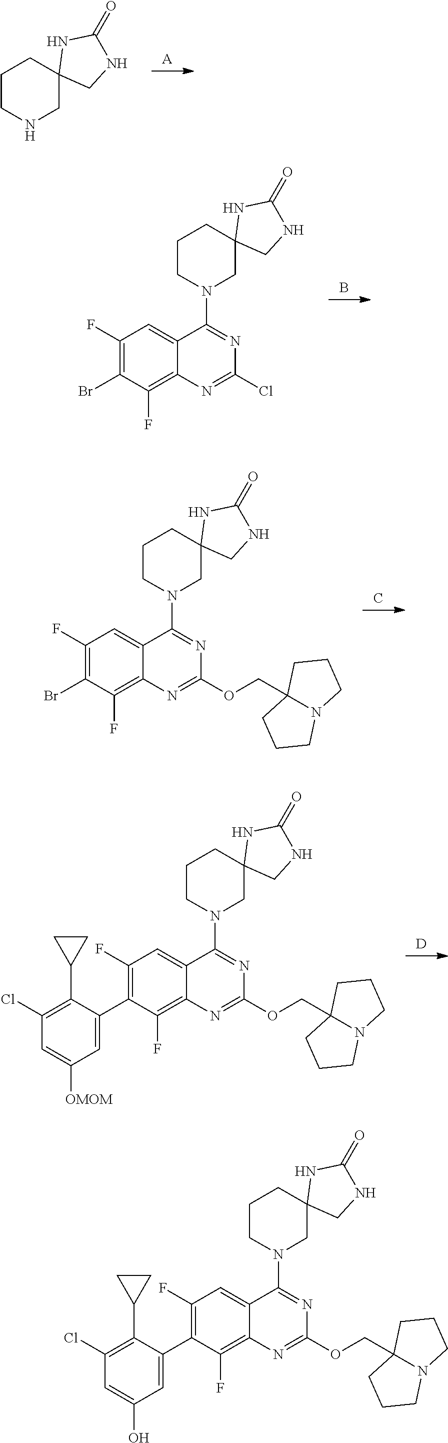

Step A. 7-(7-bromo-2-chloro-6,8-difluoroquinazolin-4-yl)-1,3,7-triazaspiro[4.5]decan-2-one: To a mixture of 7-bromo-2,4-dichloro-6,8-difluoro-quinazoline (1.25 g, 1.0 equiv) and 1,3,9-triazaspiro[4.5]decan-2-one (742 mg, 1.2 equiv) in DCM (15 mL) were added DIEA (1.54 g, 3.0 equiv) and 4 Å molecular sieve (1.50 g). The reaction was stirred at 0° C. for 0.5 hours. The mixture was diluted with ice-water (50 mL) and extracted with EtOAc (3×30 mL). The combined organic layers were washed with brine (50 mL), dried over sodium sulfate, concentrated and purified with re-crystallization from EtOAc (10 mL) at 25° C. to afford the title compound (1.60 g, 62% yield over two steps) as yellow solid; LCMS (ESI, M+1, M+3): m/z=432.1, 434.1.

Step B. 7-(7-bromo-6,8-difluoro-2-((hexahydro-1H-pyrrolizin-7a-yl)methoxy)quinazolin-4-yl)-1,3,7-triazaspiro[4.5]decan-2-one: A mixture of 7-(7-bromo-2-chloro-6,8-difluoroquinazolin-4-yl)-1,3,7-triazaspiro[4.5]decan-2-one (500 mg, 1.0 equiv) and 1,2,3,5,6,7-hexahydropyrrolizin-8-ylmethanol (2.45 g, 15.0 equiv) was stirred at 80° C. for 12 hours. The mixture was purified with prep-HPLC [column: 3_Phenomenex Luna C18 75×30 mm×3 μm; mobile phase: water (FA)−ACN; B %: 13%-33% over 8 minutes] to afford the title compound (120 mg, 19% yield) as yellow solid; LCMS (ESI, M+1, M+3): m/z=537.2, 539.2.

Step C. 7-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-1,3,7-triazaspiro[4.5]decan-2-one: To a mixture of 7-(7-bromo-6,8-difluoro-2-((hexahydro-1H-pyrrolizin-7a-yl)methoxy)quinazolin-4-yl)-1,3,7-triazaspiro[4.5]decan-2-one (100 mg, 1.0 equiv) and 2-(3-chloro-2-cyclopropyl-5-(methoxymethoxy)phenyl)-4,4,5,5-tetramethyl-1,3,2-dioxaborolane (75.6 mg, 1.2 equiv) in DMF (1 mL) and H₂O (0.2 mL) were added K₃PO₄ (119 mg, 3.0 equiv) and CataCXium Pd G3 (13.6 mg, 0.1 equiv). The reaction was degassed and purged with nitrogen for 3 times. The reaction was stirred at 70° C. for 3 hours. The mixture was diluted with ice-water (10 mL) and extracted with EtOAc (3×20 mL). The combined organic layers were washed with brine (30 mL), dried over sodium sulfate, concentrated and purified with prep-HPLC [column: Phenomenex Luna C18 150×25 mm×10 μm; mobile phase: water(FA)−ACN; B %: 21%-51% over 10 minutes] to afford the title compound (30.0 mg, 24% yield) as white solid; LCMS (ESI, M+1): m/z=669.4.

Step D. 7-(7-(3-chloro-2-cyclopropyl-5-hydroxyphenyl)-6,8-difluoro-2-((hexahydro-1H-pyrrolizin-7a-yl)methoxy)quinazolin-4-yl)-1,3,7-triazaspiro[4.5]decan-2-one: To a solution of 7-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-1,3,7-triazaspiro[4.5]decan-2-one (20.0 mg, 1.0 equiv) in MeCN (1.0 mL) was added HCl.MeOH (4 M, 1.0 mL) at 0° C. The reaction was stirred at 0° C. for 1 hour. The mixture was concentrated and purified with prep-HPLC [column: Waters Xbridge 150×25 mm×5 μm; mobile phase: water (NH₄HCO₃)−ACN; B %: 44%-74% over 8 minutes] to afford the title compound (4.81 mg, 26% yield) as white solid; ¹H NMR (400 MHz, METHANOL-d₄) δ=7.39-7.54 (m, 1H), 6.78 (d, J=2.4 Hz, 1H), 6.49 (d, J=2.4 Hz, 1H), 4.36-4.48 (m, 1H), 4.14 (q, J=10.8 Hz, 2H), 3.52-3.79 (m, 4H), 3.26 (dd, J=9.2, 5.2 Hz, 1H), 2.94-3.06 (m, 2H), 2.55-2.68 (m, 2H), 1.89-2.02 (m, 2H), 1.68-1.89 (m, 8H), 1.51-1.68 (m, 3H), 0.37-0.50 (m, 2H), 0.00 (br t, J=4.0 Hz, 2H); LCMS (ESI, M+1): m/z=625.4.

### Example 94

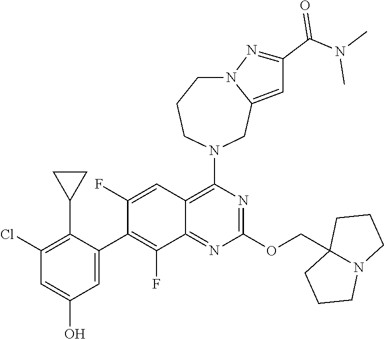 [SMILES: CN(C)C(=O)c1cc2n(n1)CCCN(c1nc(OCC34CCCN3CCC4)nc3c(F)c(-c4cc(O)cc(Cl)c4C4CC4)c(F)cc13)C2]

### 5-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

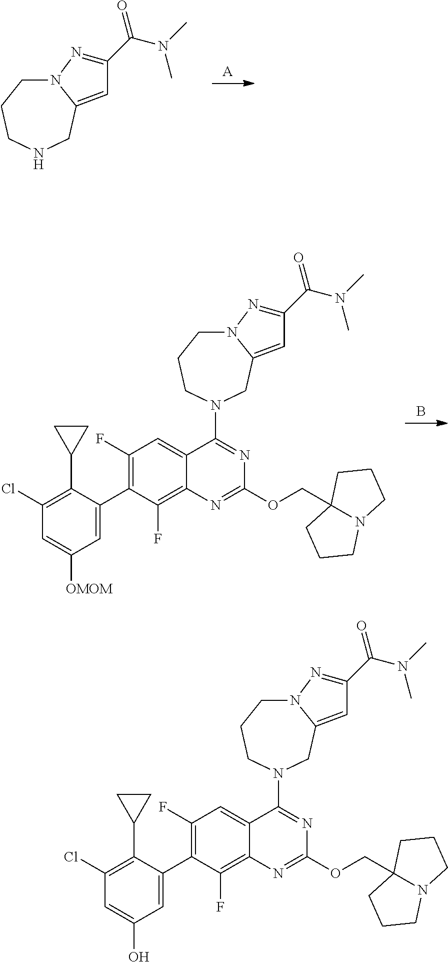

Step A. 5-(7-(3-chloro-2-cyclopropyl-5-(methoxymethoxy)phenyl)-6,8-difluoro-2-((tetrahydro-1H-pyrrolizin-7a(5H)yl)methoxy)guinazolin-4-yl)-N,N-dimethyl-5,6,7,8-tetrahydro-4H-pyrazolo[1,5-a][1,4]diazepine-2-carboxamide: To a mixture of N,N-dimethyl-5,6,7,8-tetrahydro-4H-pyrazolo[1,5-a][1,4]diazepine-2-carboxamide hydrogen chloride (90.0 mg, 7.5 equiv) and 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline (30.0 mg, 1.0 equiv) in DMF (2.00 mL) was added K₃PO₄ (41.5 mg, 4.0 equiv). The reaction was stirred at 120° C. for 24 hours. The mixture was filtered and purified with prep-HPLC [column: Unisil 3-100 C18 Ultra 150×50 mm×3 μm; mobile phase: water (FA)−ACN; B %: 26%-56% over 7 minutes] to afford the title compound (15.0 mg, 38% yield) as white solid; LCMS (ESI, M+1): m/z=722.4.

Step B. 5-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of 5-(7-(3-chloro-2-cyclopropyl-5-(methoxymethoxy)phenyl)-6,8-difluoro-2-((tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide (10.0 mg, 1.0 equiv) in MeOH (0.5 mL) was added HCl.MeOH (4 M, 0.5 mL). The reaction was stirred at 0° C. for 1 hour. The mixture was concentrated under vacuum. The residue was diluted with water (0.5 mL). The mixture was adjusted to pH=7 with saturated NaHCO₃(2 mL) and extracted with EtOAc (2×2 mL). The organic layers were dried over anhydrous sodium sulfate, concentrated and purified with prep-HPLC [column: Unisil 3-100 C18 Ultra 150×50 mm×3 μm; mobile phase: water (FA)−ACN; B %: 17%-47% over 7 minutes] to afford the title compound (5.00 mg, 51% yield, HCOOH) as white solid; ¹H NMR (400 MHz, DMSO-d₆) δ=7.60 (d, J=8.8 Hz, 1H), 6.95 (d, J=2.4 Hz, 1H), 6.63 (d, J=2.0 Hz, 1H), 6.49-6.43 (m, 1H), 4.35-4.28 (m, 2H), 4.13 (d, J=12.0 Hz, 1H), 4.03-3.96 (m, 1H), 3.83 (s, 2H), 3.79-3.72 (m, 1H), 3.25 (s, 3H), 3.05-3.00 (m, 2H), 2.93 (s, 3H), 2.37-2.27 (m, 3H), 2.01-1.90 (m, 3H), 1.85-1.30 (m, 9H), 0.64-0.56 (m, 2H), 0.16-0.06 (m, 2H); LCMS (ESI, M+1): m/z=678.3.

### Example 95

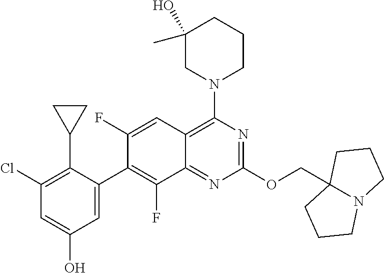 [SMILES: C[C@@]1(O)CCCN(c2nc(OCC34CCCN3CCC4)nc3c(F)c(-c4cc(O)cc(Cl)c4C4CC4)c(F)cc23)C1]

### (3R)-1-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-3-methylpiperidin-3-ol

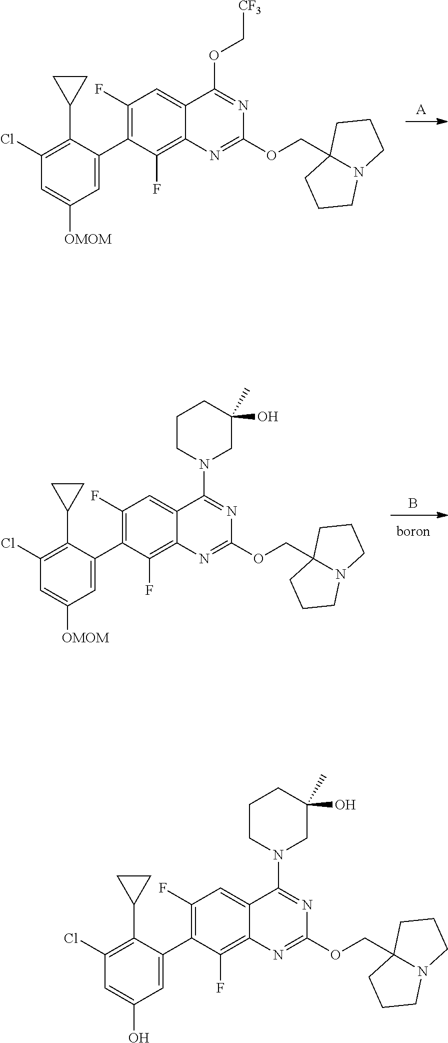

Step A. (3R)-1-(7-(3-chloro-2-cyclopropyl-5-(methoxymethoxy)phenyl)-6,8-difluoro-2-((tetrahydro-1H-pyrrolizin-7a(5H)-yl)methoxy)guinazolin-4-yl)-3-methylpiperidin-3-ol: To a mixture of 7-(3-chloro-2-cyclopropyl-5-(methoxymethoxy)phenyl)-6,8-difluoro-2-((tetrahydro-1H-pyrrolizin-7a(5H)-yl)methoxy)-4-(2,2,2-trifluoroethoxy)quinazoline (60.0 mg, 1.0 equiv) and (3R)-3-methylpiperidin-3-ol (20.3 mg, 1.8 equiv) in DMF (1.50 mL) was added K₃PO₄ (41.5 mg, 2.0 equiv) and 4 Å molecular sieve (30.0 mg). The reaction was stirred at 110° C. for 16 hours. The mixture was diluted with water (100 mL) and exacted with EtOAc (80 mL). The combined organic layers were washed with brine (100 mL), dried over anhydrous sodium sulfate, concentrated, and purified with prep-HPLC [FA condition: column: Phenomenex Luna C18 150×25 mm×10 μm; mobile phase: [water (FA)−ACN]; B %: 21%-51%, 10 mins] to afford the title compound (45.0 mg, 73% yield) as white solid; LCMS (ESI, M+1): m/z=629.5.

Step B. (3R)-1-(7-(3-chloro-2-cyclopropyl-5-hydroxyphenyl)-6,8-difluoro-2-((tetrahydro-1H-pyrrolizin-7a(5H)-yl)methoxy)guinazolin-4-yl)-3-methylpiperidin-3-ol: To a mixture of (3R)-1-(7-(3-chloro-2-cyclopropyl-5-(methoxymethoxy)phenyl)-6,8-difluoro-2-((tetrahydro-1H-pyrrolizin-7a(5H)-yl)methoxy)quinazolin-4-yl)-3-methylpiperidin-3-ol (40.0 mg, 1.0 equiv) in MeOH (2.0 Ml) was added HCl.MeOH (4M, 2.0 mL). The reaction was stirred at 0° C. for 0.5 hours. The mixture was concentrated under vacuum. The residue was diluted with water (1 mL). The mixture was adjusted to pH=7 with saturated NaHCO₃(2 mL) and extracted with EtOAc (2×2 mL). The organic layers were dried over anhydrous sodium sulfate, concentrated and purified with prep-HPLC [FA condition: column: Phenomenex luna C18 150×25 mm×10 μm; mobile phase: [water (FA)−ACN]; B %: 11%-41%, 10 mins] to afford the title compound (7.50 mg, 96% purity, HCOOH) as white solid; ¹H NMR (400 MHz, DMSO-d₆) δ=7.60 (d, J=8.8 Hz, 1H), 6.95 (d, J=2.4 Hz, 1H), 6.63 (d, J=2.4 Hz, 1H), 4.14 (br d, J=12.4 Hz, 1H), 4.00 (d, J=12.4 Hz, 1H), 3.80-3.70 (m, 1H), 3.32-3.25 (m, 2H), 2.38-2.28 (m, 3H), 2.27-2.16 (m, 2H), 2.11-1.90 (m, 4H), 1.89-1.72 (m, 1H), 1.70-1.57 (m, 4H), 1.52-1.38 (m, 3H), 1.38-1.15 (m, 2H), 1.10 (s, 3H), 0.60 (br d, J=8.0 Hz, 2H), 0.10 (br d, J=4.0 Hz, 2H); LCMS (ESI, M+1): m/z=585.4.

### Example 96

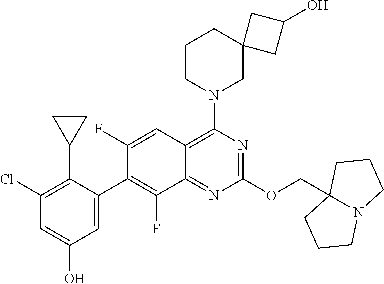 [SMILES: Oc1cc(Cl)c(C2CC2)c(-c2c(F)cc3c(N4CCCC5(CC(O)C5)C4)nc(OCC45CCCN4CCC5)nc3c2F)c1]

### 6-(7-(3-chloro-2-cyclopropyl-5-hydroxyphenyl)-6,8-difluoro-2-((tetrahydro-1H-pyrrolizin-7a(5H)-yl)methoxy)quinazolin-4-yl)-6-azaspiro[3.5]nonan-2-ol

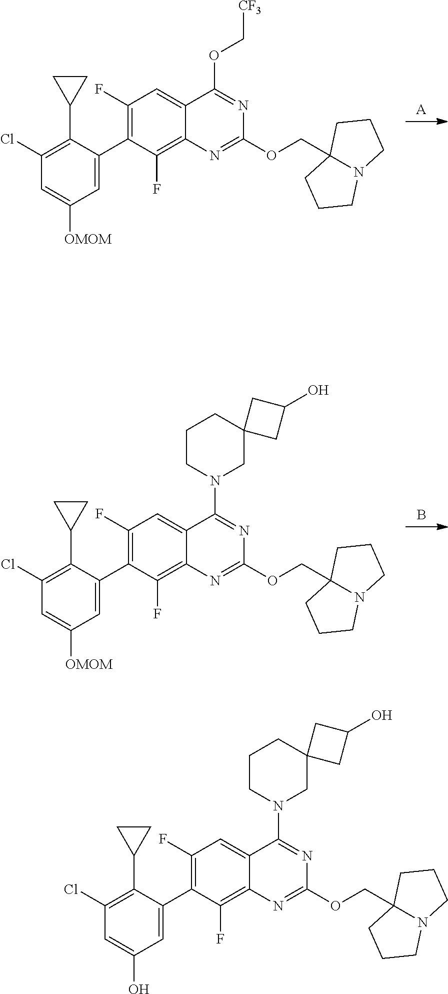

Step A. 6-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-6-azaspiro[3.5]nonan-2-ol: To a solution of 7-(3-chloro-2-cyclopropyl-5-(methoxymethoxy)phenyl)-6,8-difluoro-2-((hexahydro-1H-pyrrolizin-7a-yl)methoxy)-4-(2,2,2-trifluoroethoxy)quinazoline (33.0 mg, 1.0 equiv) in DMF (2.00 mL) were added K₃PO₄ (34.5 mg, 2.0 equiv) and 6-azaspiro[3.5]nonan-2-ol (23.0 mg, 2.0 equiv). The reaction was stirred at 100° C. for 12 hours. The mixture was diluted with water (2 mL) and extracted with EtOAc (2×3 mL). The combined organic layers were washed with brine (3 mL), dried over sodium sulfate, concentrated and purified with column chromatography [SiO₂, PE/EtOAc=1/1 to 0/1] to afford the title compound (30.0 mg, 45% yield) as yellow solid; LCMS (ESI, M+1): m/z=655.2.

Step B. 6-(7-(3-chloro-2-cyclopropyl-5-hydroxyphenyl)-6,8-difluoro-2-((hexahydro-1H-pyrrolizin-7a-yl)methoxy)quinazolin-4-yl)-6-azaspiro[3.5]nonan-2-ol: To a solution of 6-(7-(3-chloro-2-cyclopropyl-5-(methoxymethoxy)phenyl)-6,8-difluoro-2-((hexahydro-1H-pyrrolizin-7a-yl)methoxy)quinazolin-4-yl)-6-azaspiro[3.5]nonan-2-ol (30.0 mg, 1.0 equiv) in dioxane (2.0 mL) was added HCl.dioxane (4 M, 2.0 mL). The reaction was stirred at 0° C. for 1 hour. The mixture was concentrated and purified with prep-HPLC [column: Waters Xbridge 150×25 mm×5 μm; mobile phase: [water (ammonia hydroxide v/v)−ACN]; B %: 27%-57%, 9 mins] to afford the title compound (6.00 mg, 21% yield) as white solid; ¹H NMR (400 MHz, DMSO-d₆) δ=7.59 (br d, J=9.2 Hz, 1H), 6.91 (br s, 1H), 6.58 (br s, 1H), 4.17-3.96 (m, 3H), 3.82-3.72 (m, 1H), 2.31-1.88 (m, 12H), 1.75-1.26 (m, 13H), 0.59 (br d, J=7.6 Hz, 2H), 0.12 (br s, 2H); LCMS (ESI, M+1): m/z=611.4.

### Example 97

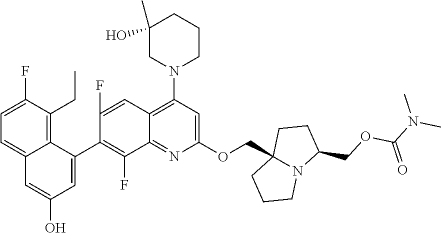 [SMILES: CCc1c(F)ccc2cc(O)cc(-c3c(F)cc4c(N5CCC[C@@](C)(O)C5)cc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate

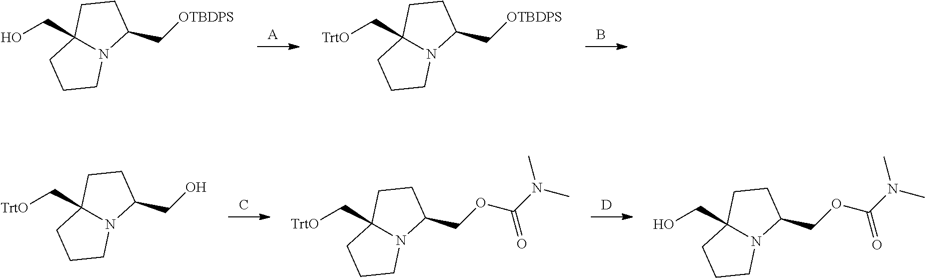

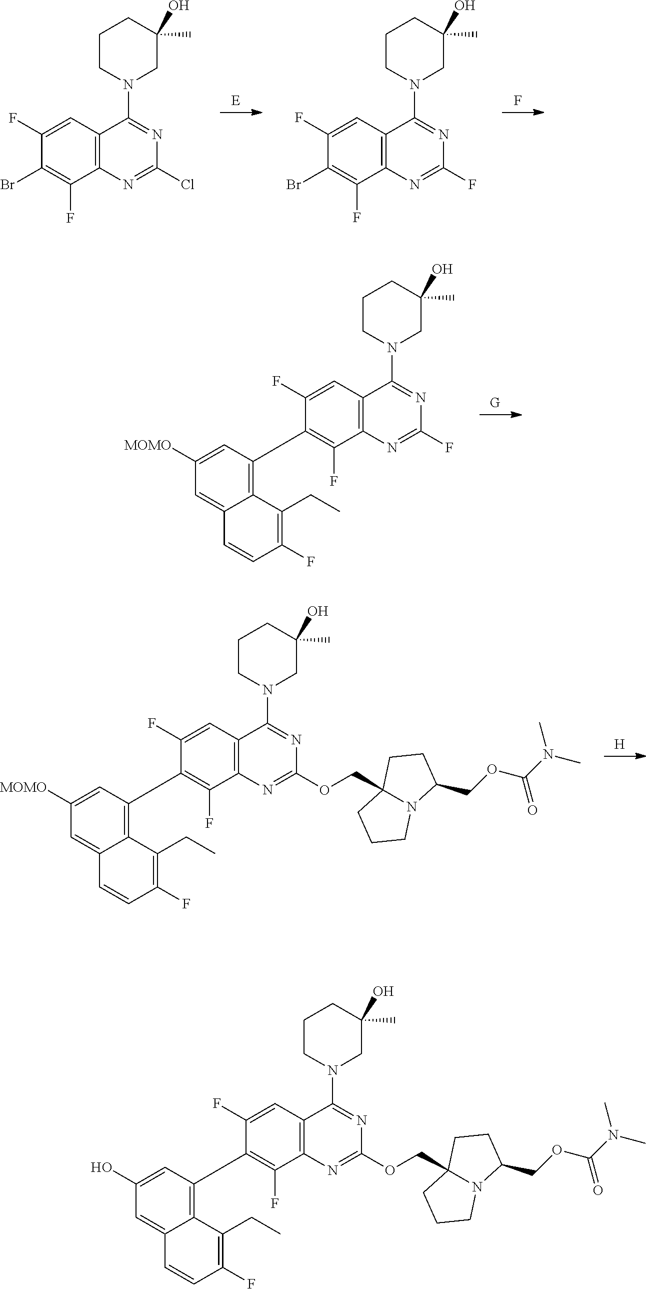

Step A. (3 S,7aR)-3-(((tert-butyldiphenylsilyl)oxy)methyl)-7a-((trityloxy)methyl)hexahydro-1H-pyrrolizine: To a mixture of ((3S,7aR)-3-(((tert-butyldiphenylsilyl)oxy)methyl)hexahydro-1H-pyrrolizin-7a-yl)methanol (6.44 g, 1.0 equiv) and TEA (3.98 g, 2.5 equiv) in DCM (64.4 mL) was added TrtCl (8.77 g, 2.0 equiv) at 0° C. The reaction was stirred at 15° C. for 12 hours. The mixture was diluted with water (100 mL) and extracted with DCM (3×50 mL). The combined organic layers were washed with brine (2×40 mL), dried over anhydrous sodium sulfate, filtered and concentrated to afford the title compound (10.3 g, crude) as yellow oil; LCMS (ESI, M+1): m/z=652.8.

Step B. ((3S,7aR)-7a-((trityloxy)methyl)hexahydro-1H-pyrrolizin-3-yl)methanol: To a solution of (3S,7aR)-3-(((tert-butyldiphenylsilyl)oxy)methyl)-7a-((trityloxy)methyl)hexahydro-1H-pyrrolizine (10.3 g, 1.0 equiv) in DMF (20.3 mL) was added CsF (23.9 g, 10 equiv). The reaction was stirred at 25° C. for 12 hours. The mixture was diluted with water (60.0 mL) and extracted with EtOAc (3×20 mL). The combined organic layers were washed with brine (2×20 mL), dried over anhydrous sodium sulfate, concentrated and purified with reversed-phase flash [0.1% FA condition] to afford the title compound (4.15 g, two steps 57% yield) as yellow oil; ¹H NMR (400 MHz, CDCl₃) δ=7.49-7.38 (m, 6H), 7.29-7.17 (m, 9H), 3.44 (dd, J=4.4, 10.4 Hz, 1H), 3.27 (br dd, J=3.6, 10.8 Hz, 1H), 2.95-2.83 (m, 3H), 2.82-2.73 (m, 1H), 2.62 (td, J=6.0, 11.2 Hz, 1H), 2.02 (s, 1H), 1.89-1.81 (m, 1H), 1.78-1.48 (m, 6H).

Step C. ((3S,7aR)-7a-((trityloxy)methyl)hexahydro-1H-pyrrolizin-3-yl)methyl dimethylcarbamate: To a mixture of ((3S,7aR)-7a-((trityloxy)methyl)hexahydro-1H-pyrrolizin-3-yl)methanol (4.15 g, 1.0 equiv) and TEA (3.05 g, 3.0 equiv) in DCM (42 mL) was added (4-nitrophenyl) carbonochloridate (3.03 g, 1.5 equiv) at 0-5° C. under nitrogen. The reaction was stirred at 20° C. for 2 hours. Then N-methylmethanamine (2.0 M, 7.50 mL, 1.5 equiv) was added to above mixture at 0° C. under nitrogen. The reaction was stirred at 0° C. for another 0.5 hours. The mixture was diluted with water (50.0 mL) and extracted with DCM (3×50 mL). The combined organic layers were washed with brine (50 mL), dried over anhydrous sodium sulfate, concentrated and purified with reversed-phase flash [0.1% FA condition] to afford the title compound (2.03 g, 41% yield) as yellow oil; LCMS (ESI, M+1): m/z=485.7.

Step D. ((3S,7aR)-7a-(hydroxymethyl)hexahydro-1H-pyrrolizin-3-yl)methyl dimethylcarbamate: To a solution of ((3S,7aR)-7a-((trityloxy)methyl)hexahydro-1H-pyrrolizin-3-yl)methyl dimethylcarbamate (2.03 g, 1.0 equiv) in DCM (20.0 mL) was added TFA (4.78 g, 10 equiv). The reaction was stirred at 20° C. for 12 hours. The mixture was concentrated. The residue was dissolved in MeOH (10 mL). Then the mixture was neutralized with NaHCO₃ solid and purified with column chromatography [Al₂O₃, DCM/MeOH=15:1] to afford the title compound (834 mg, 82% yield) as yellow oil; ¹H NMR (400 MHz, METHANOL-d4) δ=4.03-3.93 (m, 2H), 3.41-3.18 (m, 3H), 3.04-2.97 (m, 1H), 2.97-2.84 (m, 6H), 2.80 (td, J=4.8, 10.4 Hz, 1H), 2.09-1.93 (m, 2H), 1.91-1.82 (m, 1H), 1.82-1.71 (m, 2H), 1.69-1.47 (m, 3H).

Step E. (R)-1-(7-bromo-2,6,8-trifluoroquinazolin-4-yl)-3-methylpiperidin-3-ol: To a solution of KF (160 mg, 6.0 equiv) in DMSO (1.80 mL) was added (R)-1-(7-bromo-2-chloro-6,8-difluoroquinazolin-4-yl)-3-methylpiperidin-3-ol (180 mg, 1.0 equiv). The reaction was stirred at 120° C. for 6 hours. The mixture was diluted with water (6.00 mL) and extracted with EtOAc (3×3.0 mL). The combined organic layers were washed with brine (2×5 mL), dried over anhydrous sodium sulfate, concentrated and purified with reversed-phase flash [0.1% FA condition] to afford the title compound (60.0 mg, 33% yield) as white solid; LCMS (ESI, M+1, M+3): m/z=375.7, 377.7.

Step F. (3R)-1-(7-(8-ethyl-7-fluoro-3-(methoxymethoxy)naphthalen-1-yl)-2,6,8-trifluoroquinazolin-4-yl)-3-methylpiperidin-3-ol: To a mixture of (R)-1-(7-bromo-2,6,8-trifluoroquinazolin-4-yl)-3-methylpiperidin-3-ol (40.0 mg, 1.0 equiv) and 2-(8-ethyl-7-fluoro-3-(methoxymethoxy)naphthalen-1-yl)-4,4,5,5-tetramethyl-1,3,2-dioxaborolane (42.1 mg, 1.1 equiv) in cyclopentyl methyl ether (2.00 mL) were added K₃PO₄ (1.5 M, 0.40 mL, 5.6 equiv) and CataCXium A Pd G3 (7.74 mg, 0.10 equiv). The reaction was degassed and purged with nitrogen for 3 times. The reaction was stirred at 90° C. for 2 hours. The mixture was diluted with water (3.00 mL) and extracted with EtOAc (3×5 mL). The combined organic layers were washed with brine (10 mL), dried over anhydrous sodium sulfate, concentrated and purified by column chromatography [Al₂O₃, PE/EtOAc=10/1 to 4/1] to afford the title compound (45.0 mg, 52% yield) as yellow oil; LCMS (ESI, M+1): m/z=530.2.

Step G. ((3S,7aR)-7a-(((7-(8-ethyl-7-fluoro-3-(methoxymethoxy)naphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate: To a mixture of (3R)-1-(7-(8-ethyl-7-fluoro-3-(methoxymethoxy)naphthalen-1-yl)-2,6,8-trifluoroquinazolin-4-yl)-3-methylpiperidin-3-ol (50.0 mg, 1.0 equiv) and ((3S,7aR)-7a-(hydroxymethyl)hexahydro-1H-pyrrolizin-3-yl)methyl dimethylcarbamate (22.9 mg, 1.0 equiv) in THE (0.6 mL) was added t-BuONa (2.0 M, 0.19 mL, 4.0 equiv) at 0-5° C. The reaction was stirred at 0-5° C. for 2 hours. The mixture was diluted with water (2.00 mL) and extracted with EtOAc (3×2.0 mL). The combined organic layers were washed with brine (5 mL), dried over anhydrous sodium sulfate, filtered and concentrated to afford the title compound (35 mg, crude) as yellow oil; LCMS (ESI, M+1): m/z=751.8.

Step H. ((3S,7aR)-7a-(((7-(8-ethyl-7-fluoro-3-hydroxynaphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate: To a mixture of ((3S,7aR)-7a-(((7-(8-ethyl-7-fluoro-3-(methoxymethoxy)naphthalen-1-yl)-6,8-difluoro-4-((R)-3-hydroxy-3-methylpiperidin-1-yl)quinazolin-2-yl)oxy)methyl)hexahydro-1H-pyrrolizin-3-yl)methyl dimethylcarbamate (30.0 mg, 1.0 equiv) in MeCN (0.30 mL) was added HCl.dioxane (4M, 0.30 mL) at 0-5° C. The reaction was stirred at for 1 hour. The mixture was concentrated and purified by reversed-phase HPLC [column: Waters xbridge 150×25 mm 10 μm; mobile phase: [water (NH₄HCO₃)−ACN]; B %: 50%-80%, 10 mins] to afford the title compound (2.00 mg, 6.9% yield) as yellow solid; ¹H NMR (400 MHz, METHANOL-d₄) δ=7.79 (br d, J=11.2 Hz, 1H), 7.69 (dd, J=6.0, 9.2 Hz, 1H), 7.31 (d, J=2.8 Hz, 1H), 7.27 (t, J=9.6 Hz, 1H), 7.01 (d, J=2.4 Hz, 1H), 4.35-4.19 (m, 3H), 4.16-4.03 (m, 2H), 3.99 (br dd, J=6.8, 11.2 Hz, 1H), 3.58-3.49 (m, 1H), 3.47-3.37 (m, 2H), 3.20-3.02 (m, 2H), 2.92 (br d, 6H), 2.63-2.53 (m, 1H), 2.50-2.41 (m, 1H), 2.30-2.15 (m, 2H), 2.10-2.02 (m, 1H), 2.01-1.91 (m, 3H), 1.89-1.66 (m, 6H), 1.29 (s, 3H), 0.83 (t, J=7.2 Hz, 3H); LCMS (ESI, M+1): m/z=708.2.

### Example 98

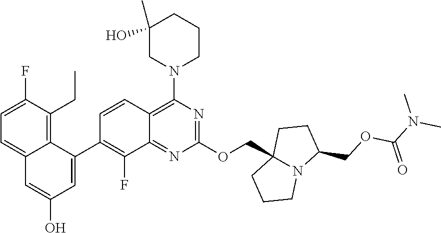 [SMILES: CCc1c(F)ccc2cc(O)cc(-c3ccc4c(N5CCC[C@@](C)(O)C5)nc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-((R)-3-hydroxy-3-methylpiperidin-1-yl)quinazolin-2-yl)oxy)methyl)hexahydro-1H-pyrrolizin-3-yl)methyl dimethylcarbamate

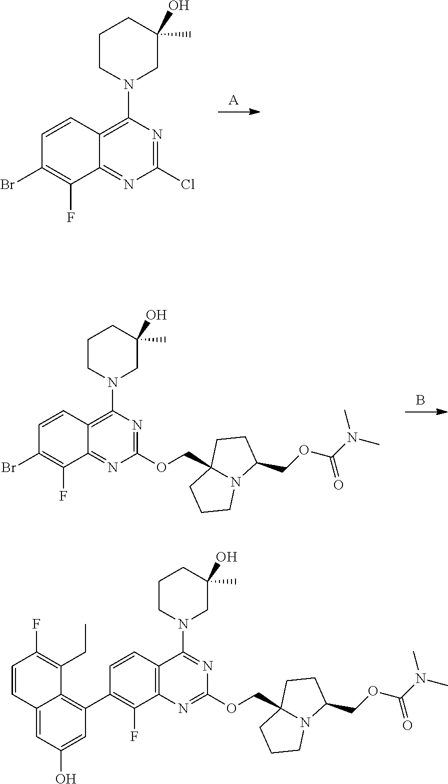

Step A. ((3S,7aR)-7a-(((7-bromo-8-fluoro-4-((R)-3-hydroxy-3-methylpiperidin-1-yl)quinazolin-2-yl)oxy)methyl)hexahydro-1H-pyrrolizin-3-yl)methyl dimethylcarbamate: To a solution of ((3S,7aR)-7a-(hydroxymethyl)hexahydro-1H-pyrrolizin-3-yl)methyl dimethylcarbamate (97.0 mg, 1.0 equiv) in DMAc (3.00 mL) was added NaH (64.1 mg, 60% purity, 4.0 equiv). The reaction was stirred at 0° C. for 1 hour. Then (R)-1-(7-bromo-2-chloro-8-fluoroquinazolin-4-yl)-3-methylpiperidin-3-ol (150 mg, 1.0 equiv) was added dropwise to give a colorless solution. The reaction was stirred at 20° C. for another 1 hour. The mixture was diluted with water (20.0 mL) and extracted with EtOAc (3×30 mL). The combined organic layers were washed with brine, dried over anhydrous sodium sulfate, concentrated and purified by prep-HPLC [column: Phenomenex luna C18 150×25 mm×10 μm; mobile phase: [[water(NH₃H₂O)−ACN];B %: 45%-75%, 8 mins] to afford the title compound (47 mg, 20% yield) as white solid; LCMS (ESI, M+1, M+3): m/z=580.3, 582.3.

Step B. ((3S,7aR)-7a-(((7-(8-ethyl-7-fluoro-3-hydroxynaphthalen-1-yl)-8-fluoro-4-((R)-3-hydroxy-3-methylpiperidin-1-yl)quinazolin-2-yl)oxy)methyl)hexahydro-1H-pyrrolizin-3-yl)methyl dimethylcarbamate: To a mixture of ((3S,7aR)-7a-(((7-bromo-8-fluoro-4-((R)-3-hydroxy-3-methylpiperidin-1-yl)quinazolin-2-yl)oxy)methyl)hexahydro-1H-pyrrolizin-3-yl)methyl dimethylcarbamate (27.0 mg, 1.0 equiv) and 5-ethyl-6-fluoro-4-(4,4,5,5-tetramethyl-1,3,2-dioxaborolan-2-yl)naphthalen-2-ol (14.7 mg, 1.0 equiv) in THE (0.5 mL) were added CataCXium A Pd G3 (6.77 mg, 0.2 equiv) and K₃PO₄ (1.5 M, 3.0 equiv). The reaction was stirred at 60° C. for 2 hours. The mixture was diluted with water (20.0 mL) and extracted with EtOAc (3×30 mL). The combined organic layers were washed with brine (30.0 mL), dried over anhydrous sodium sulfate, concentrated and purified with prep-HPLC [column: Phenomenex luna C18 150×25 mm×10 μm; mobile phase: [water(FA)−ACN];B %: 30%-50%, 58 mins] to afford the title compound (4.46 mg, 14% yield, HCOOH) as white solid; ¹H NMR (400 MHz, CD₃OD) δ=7.65 (dd, J=5.6, 9.2 Hz, 1H), 7.34-7.27 (m, 1H), 7.27 (br s, 2H), 7.00-6.92 (m, 1H), 4.45-4.20 (m, 3H), 4.20-4.05 (m, 2H), 4.04-3.95 (m, 1H), 3.66-3.45 (m, 3H), 3.26-3.05 (m, 2H), 2.95-2.84 (m, 6H), 2.53-2.39 (m, 2H), 2.36-2.24 (m, 1H), 2.19-1.93 (m, 5H), 1.90-1.70 (m, 6H), 1.28 (d, J=14.4 Hz, 3H), 0.79 (dt, J=3.6, 7.2 Hz, 3H); LCMS (ESI, M+1): m/z=690.7.

### Example 99

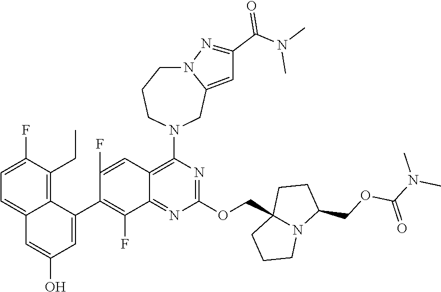 [SMILES: CCc1c(F)ccc2cc(O)cc(-c3c(F)cc4c(N5CCCn6nc(C(=O)N(C)C)cc6C5)nc(OC[C@]56CCCN5[C@H](COC(=O)N(C)C)CC6)nc4c3F)c12]

### ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-6,8-difluoroquinazolin-2-yl)oxy)methyl)hexahydro-1H-pyrrolizin-3-yl)methyl dimethylcarbamate

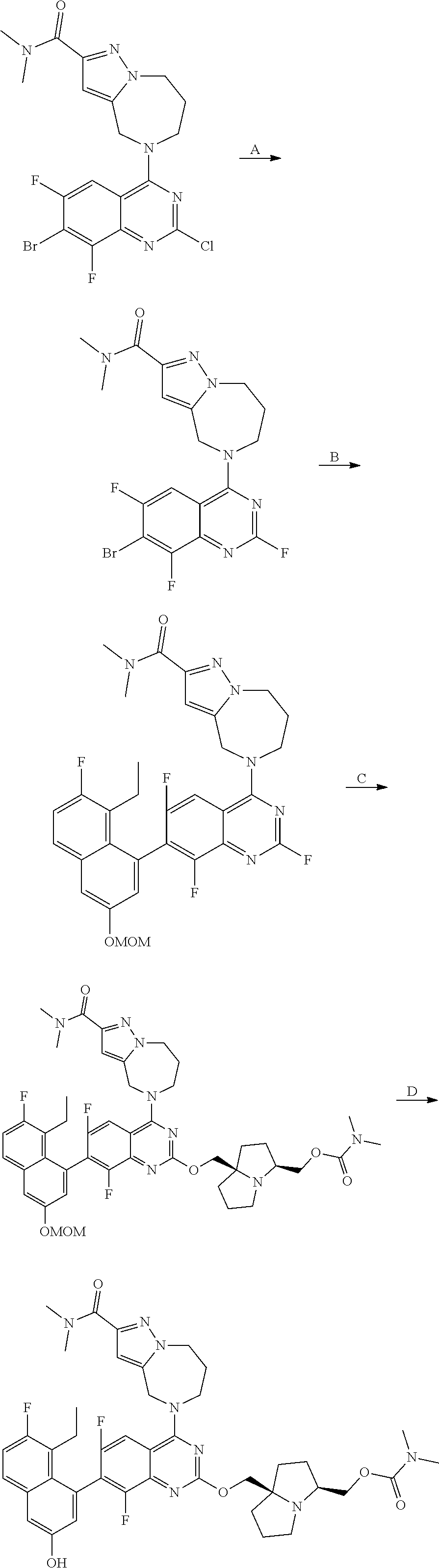

Step A. 5-(7-bromo-2,6,8-trifluoroquinazolin-4-yl)-N,N-dimethyl-5,6,7,8-tetrahydro-4H-pyrazolo[1,5-a][1,4]diazepine-2-carboxamide: To a mixture of 5-(7-bromo-2-chloro-6,8-difluoro-quinazolin-4-yl)-N,N-dimethyl-4,6,7,8-tetrahydropyrazolo[1,5-a][1,4]diazepine-2-carboxamide (750 mg, 1.0 equiv) in DMSO (8.0 mL) was added KF (538 mg, 6.0 equiv). The reaction was stirred at 120° C. for 6 hours. The mixture was diluted with water (20 mL) and extracted with EtOAc (2×20 mL). The combined organic layers were washed with brine (2×30 mL), dried over anhydrous sodium sulfate, concentrated to afford the title compound (520 mg, 66% yield) as white solid; LCMS (ESI, M+1, M+3): m/z=469.1, 471.1.

Step B. 5-(7-(8-ethyl-7-fluoro-3-(methoxymethoxy)naphthalen-1-yl)-2,6,8-trifluoroquinazolin-4-yl)-N,N-dimethyl-5,6,7,8-tetrahydro-4H-pyrazolo[1,5-a][1,4]diazepine-2-carboxamide: To a mixture of 5-(7-bromo-2,6,8-trifluoroquinazolin-4-yl)-N,N-dimethyl-5,6,7,8-tetrahydro-4H-pyrazolo[1,5-a][1,4]diazepine-2-carboxamide (500 mg, 1.0 equiv) and 2-[8-ethyl-7-fluoro-3-(methoxymethoxy)-1-naphthyl]-4,4,5,5-tetramethyl-1,3,2-dioxaborolane (1.15 g, 3.0 equiv) in THE (12.5 mL) and H₂O (2.5 mL) were added CataCXium A Pd G3 (77.6 mg, 0.1 equiv) and K₃PO₄ (1.5M, 2.5 mL, 3.5 equiv). The mixture was stirred at 60° C. for 6 hours under nitrogen atmosphere. The mixture was diluted with water (20 mL) and extracted with EtOAc (3×30 mL). The combined organic layers were washed with brine (30 mL), dried over anhydrous sodium sulfate, filtered, concentrated and purified with reversed phase flash [Phenomenex luna C18 150×25 mm×10 um; water (0.1% formic acid)/acetonitrile)] to afford the title compound (630 mg, 95% yield) as white solid; LCMS (ESI, M+1): m/z=623.3.

Step C. ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-6,8-difluoroquinazolin-2-yl)oxy)methyl)hexahydro-1H-pyrrolizin-3-yl)methyl dimethylcarbamate: To a mixture of 5-[7-[8-ethyl-7-fluoro-3-(methoxymethoxy)-1-naphthyl]-2,6,8-trifluoro-quinazolin-4-yl]-N,N-dimethyl-4,6,7,8-tetrahydropyrazolo[1,5-a][1,4]diazepine-2-carboxamide (550 mg, 1.0 equiv) and ((3S,7aR)-7a-(hydroxymethyl)hexahydro-1H-pyrrolizin-3-yl)methyl dimethylcarbamate (278 mg, 1.3 equiv) in THE (3.4 mL) was added t-BuONa (2 M, 1.5 equiv) at 0° C. under nitrogen. The reaction was stirred at 0° C. for 4 hours. The mixture was diluted with water (20 mL) and extracted with EtOAc (3×30 mL). The combined organic layers were washed with brine (30 mL), dried over anhydrous sodium sulfate, filtered, concentrated and purified with reversed phase flash [Phenomenex luna C18 150×25 mm×10 um; water (0.1% formic acid)/acetonitrile)] to afford the title compound (570 mg, 74% yield) as white solid; ¹H NMR (400 MHz, METHANOL-d₄) δ=7.89-7.74 (m, 2H), 7.64-7.61 (m, 1H), 7.32 (t, J=9.2 Hz, 1H), 7.19-7.11 (m, 1H), 6.71 (s, 1H), 5.35-5.31 (m, 2H), 5.20 (br s, 2H), 4.53-4.49 (m, 3H), 4.44-4.36 (m, 3H), 4.33-4.22 (m, 3H), 3.86-3.77 (m, 1H), 3.69-3.57 (m, 2H), 3.56-3.43 (m, 5H), 3.36-3.34 (m, 3H), 3.08 (s, 3H), 2.84 (br d, J=17.2 Hz, 3H), 2.63-2.54 (m, 1H), 2.51-2.43 (m, 2H), 2.43-2.35 (m, 2H), 2.29-2.17 (m, 4H), 2.17-2.06 (m, 3H), 0.87-0.76 (m, 3H); LCMS (ESI, M+1): m/z=845.7.

Step D. ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-6,8-difluoroquinazolin-2-yl)oxy)methyl)hexahydro-1H-pyrrolizin-3-yl)methyl dimethylcarbamate: To a solution of ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-(methoxymethoxy)naphthalen-1-yl)-6,8-difluoroquinazolin-2-yl)oxy)methyl)hexahydro-1H-pyrrolizin-3-yl)methyl dimethylcarbamate (430 mg, 1.0 equiv) in MeCN (4.3 mL) was added HCl.dioxane (4 M, 8.6 mL). The reaction was stirred at 0° C. for 1 hour. The mixture was concentrated under vacuum. The residue was diluted with water (1 mL). The mixture was adjusted to pH=7 with saturated NaHCO₃(10 mL) and extracted with EtOAc (2×10 mL). The combined organic layers were dried over anhydrous sodium sulfate, concentrated and purified with prep-HPLC (column: Phenomenex luna C18 150×25 mm×10 μm; mobile phase: [water(FA)−ACN]; B %: 22%-52% over 10 min) to afford the title compound (27.2 mg, 6.6% yield, 0.35 HCOOH) as white solid; ¹HNMR (400 MHz, METHANOL-d₄) δ=7.79-7.72 (m, 1H), 7.71-7.65 (m, 1H), 7.30 (d, J=2.8 Hz, 1H), 7.25 (t, J=9.2 Hz, 1H), 6.99 (d, J=2.4 Hz, 1H), 6.70 (s, 1H), 5.24-5.16 (m, 1H), 5.14-5.07 (m, 1H), 4.55 (br d, J=6.0 Hz, 2H), 4.39-4.26 (m, 4H), 4.23-4.15 (m, 1H), 4.10-4.02 (m, 1H), 3.34 (s, 3H), 3.27-3.15 (m, 3H), 3.08 (s, 3H), 2.94-2.88 (m, 3H), 2.85 (br s, 3H), 2.62-2.51 (m, 1H), 2.44-2.36 (m, 3H), 2.33-2.23 (m, 1H), 2.14-2.02 (m, 2H), 2.01-1.96 (m, 2H), 1.93-1.75 (m, 3H), 0.80 (t, J=7.2 Hz, 3H); LCMS (ESI, M+1): m/z=801.5.

### Example 100

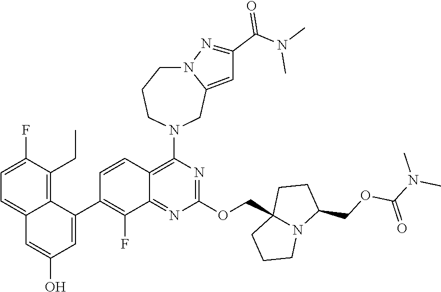 [SMILES: CCc1c(F)ccc2cc(O)cc(-c3ccc4c(N5CCCn6nc(C(=O)N(C)C)cc6C5)nc(OC[C@]56CCCN5[C@H](COC(=O)N(C)C)CC6)nc4c3F)c12]

### ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-8-fluoroquinazolin-2-yl)oxy)methyl)hexahydro-1H-pyrrolizin-3-yl)methyl dimethylcarbamate

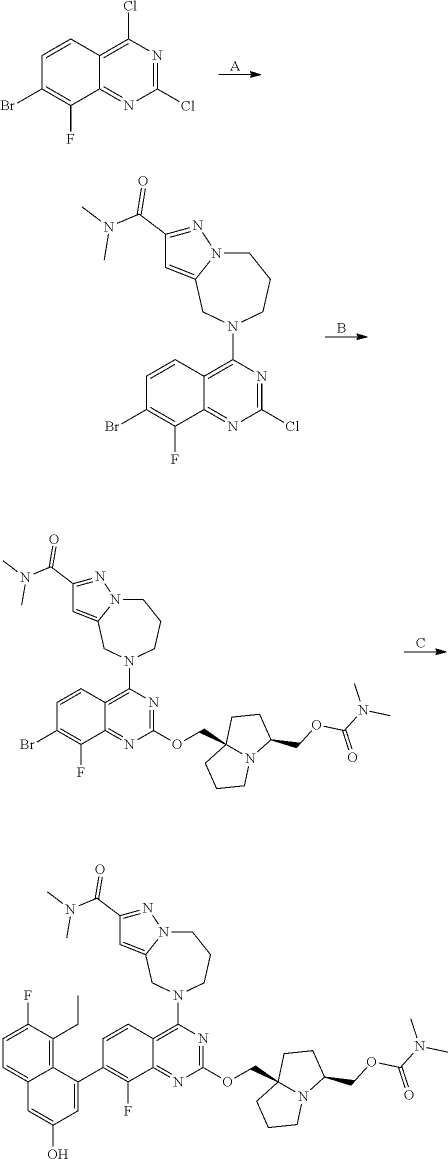

Step A. 5-(7-bromo-2-chloro-8-fluoroquinazolin-4-yl)-N,N-dimethyl-5,6,7,8-tetrahydro-4H-pyrazolo[1,5-a][1,4]diazepine-2-carboxamide: To a mixture of 7-bromo-2,4-dichloro-8-fluoroquinazoline (500 mg, 1.0 equiv) and N,N-dimethyl-5,6,7,8-tetrahydro-4H-pyrazolo[1,5-a][1,4]diazepine-2-carboxamide (352 mg, 1.0 equiv) in dioxane (10 mL) was added DIEA (655 mg, 3.0 equiv). The reaction was stirred at 50° C. for 3 hours. The mixture was filtered to afford the title compound (0.70 g, 84% yield) as white solid; ¹H NMR (400 MHz, chloroform-d) 6=7.63-7.52 (m, 2H), 6.72 (s, 1H), 4.59-4.49 (m, 2H), 4.18 (br t, J=5.6 Hz, 2H), 3.36 (s, 3H), 3.10 (s, 3H), 2.40-2.28 (m, 2H), 1.60-1.49 (m, 1H), 1.44 (d, J=6.8 Hz, 1H); LCMS (ESI, M+1, M+3): m/z=467.0, 469.0.

Step B. ((3S,7aR)-7a-(((7-bromo-4-(2-(dimethylcarbamoyl)-7,8-dihydro-4H-pyrazolo[1,5-a][1,4]diazepin-5(6H)-yl)-8-fluoroquinazolin-2-yl)oxy)methyl)hexahydro-1H-pyrrolizin-3-yl)methyl dimethylcarbamate: To a mixture of 5-(7-bromo-2-chloro-8-fluoroquinazolin-4-yl)-N,N-dimethyl-5,6,7,8-tetrahydro-4H-pyrazolo[1,5-a][1,4]diazepine-2-carboxamide (60.0 mg, 1.0 equiv) and ((3S,7aR)-7a-(hydroxymethyl)hexahydro-1H-pyrrolizin-3-yl)methyl dimethylcarbamate (40.4 mg, 1.3 equiv) in THF (1 mL) was added t-BuONa (1 M, 1.5 equiv). The reaction was stirred at 0° C. for 6 hours. The mixture was filtered and purified with reversed phase flash chromatography [C18, 0.1% formic acid condition] to afford the title compound (13.0 mg, 14% yield) as white solid; ¹H NMR (400 MHz, chloroform-d) δ=7.58-7.46 (m, 1H), 7.45-7.32 (m, 1H), 6.72-6.65 (m, 1H), 4.91-4.79 (m, 2H), 4.57-4.49 (m, 2H), 4.11-4.05 (m, 2H), 4.03-3.90 (m, 2H), 3.36 (s, 3H), 3.13-3.09 (m, 3H), 3.02-2.96 (m, 1H), 2.95-2.88 (m, 6H), 2.79-2.71 (m, 1H), 2.36-2.22 (m, 3H), 2.05-1.85 (m, 5H), 1.84-1.66 (m, 3H), 1.65-1.48 (m, 2H); LCMS (ESI, M+1, M+3): m/z=673.4, 675.4.

Step C. ((3S,7aR)-7a-(((4-(2-(dimethylcarbamoyl)-7,8-dihydro-4H-pyrazolo[1,5-a][1,4]diazepin-5(6H)-yl)-7-(8-ethyl-7-fluoro-3-hydroxynaphthalen-1-yl)-8-fluoroquinazolin-2-yl)oxy)methyl)hexahydro-1H-pyrrolizin-3-yl)methyl dimethylcarbamate: To a mixture of ((3S,7aR)-7a-(((7-bromo-4-(2-(dimethylcarbamoyl)-7,8-dihydro-4H-pyrazolo[1,5-a][1,4]diazepin-5(6H)-yl)-8-fluoroquinazolin-2-yl)oxy)methyl)hexahydro-1H-pyrrolizin-3-yl)methyl dimethylcarbamate (13.0 mg, 1.0 equiv), K₃PO₄ (1.5M, 3.0 equiv) and 5-ethyl-6-fluoro-4-(4,4,5,5-tetramethyl-1,3,2-dioxaborolan-2-yl)naphthalen-2-ol (6.10 mg, 1.0 equiv) in THE (1.0 mL) was added CataCXium A Pd G3 (1.41 mg, 0.1 equiv). The reaction was degassed and purged with nitrogen 3 times. The reaction was stirred at 60° C. for 6 hours. The mixture was diluted with water (3.0 mL) and extracted with EtOAc (2×5 mL). The combined organic layers were dried over anhydrous sodium sulfate, concentrated and purified with prep-HPLC (column: Waters xbridge 150×25 mm 10 um; mobile phase: [water (NH₄HCO₃)−ACN];B %: 38%-68%,8 min) to afford the title compound (3.84 mg, 24% yield) as yellow solid; ¹H NMR (400 MHz, METHANOL-d₄) δ=7.91 (d, J=8.8 Hz, 1H), 7.65 (dd, J=6.0, 9.2 Hz, 1H), 7.32 (dd, J=6.8, 8.8 Hz, 1H), 7.24 (d, J=2.4 Hz, 1H), 7.23-7.19 (m, 1H), 6.95 (t, J=2.4 Hz, 1H), 6.71-6.69 (m, 1H), 5.20 (br d, J=8.0 Hz, 1H), 5.14 (br d, J=8.0 Hz, 1H), 4.60 (br s, 2H), 4.41-4.25 (m, 2H), 4.15 (s, 2H), 4.10-4.03 (m, 1H), 4.01-3.95 (m, 1H), 3.33 (s, 3H), 3.11-2.99 (m, 5H), 2.91 (br d, J=17.2 Hz, 6H), 2.85-2.76 (m, 1H), 2.56-2.45 (m, 1H), 2.43-2.35 (m, 3H), 2.25 (s, 1H), 2.05-1.99 (m, 1H), 1.87 (br s, 3H), 1.84-1.73 (m, 2H), 1.71-1.62 (m, 1H), 0.77 (t, J=7.2 Hz, 3H); LCMS (ESI, M+1): m/z=783.6.

### Example 101

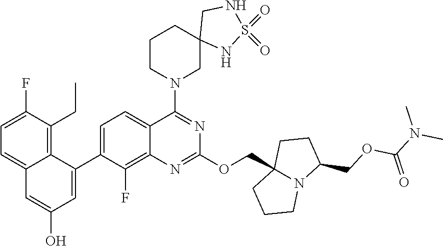 [SMILES: CCc1c(F)ccc2cc(O)cc(-c3ccc4c(N5CCCC6(CNS(=O)(=O)N6)C5)nc(OC[C@]56CCCN5[C@H](COC(=O)N(C)C)CC6)nc4c3F)c12]

### ((3S,7aR)-7a-(((4-(2,2-dioxido-2-thia-1,3,7-triazaspiro[4.5]decan-7-yl)-7-(8-ethyl-7-fluoro-3-hydroxynaphthalen-1-yl)-8-fluoroquinazolin-2-yl)oxy)methyl)hexahydro-1H-pyrrolizin-3-yl)methyl dimethylcarbamate

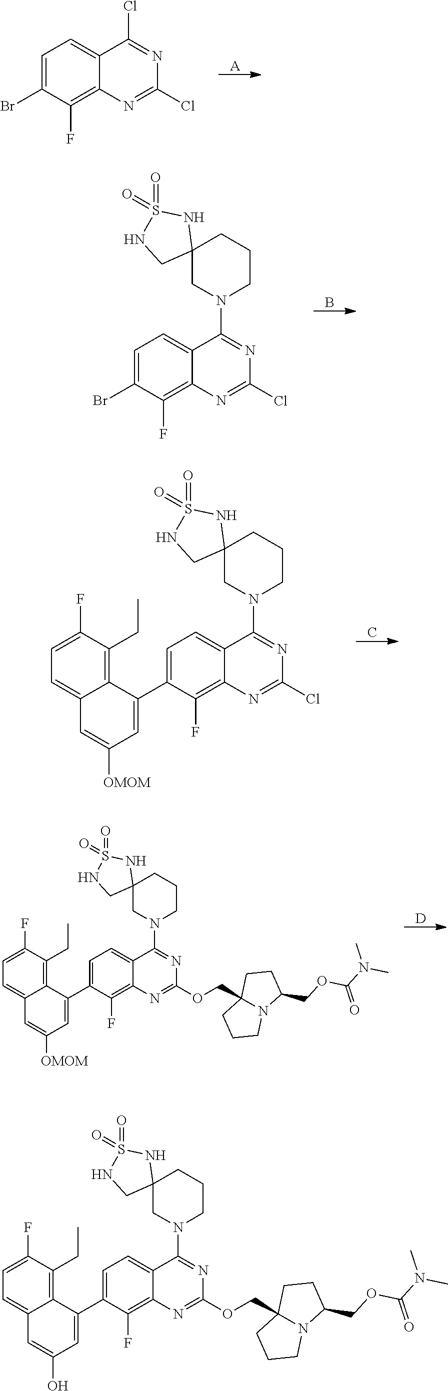

Step A. 7-(7-bromo-2-chloro-8-fluoroquinazolin-4-yl)-2-thia-1,3,7-triazaspiro[4.5]decane 2,2-dioxide: To a mixture of 7-bromo-2,4-dichloro-8-fluoroquinazoline (1.00 g, 1.0 equiv) and 2-thia-1,3,7-triazaspiro[4.5]decane 2,2-dioxide (646 mg, 1.0 equiv) in DCM (10 mL) was added DIEA (1.31 g, 3.0 equiv). The reaction was stirred at 25° C. for 2 hours. The mixture was filtered and the filter cake was collected to afford the title compound (1.33 g, 84% yield) as white solid; ¹H NMR (400 MHz, DMSO-d₆) δ=7.83 (br d, J=9.2 Hz, 1H), 7.76-7.66 (m, 1H), 7.26 (s, 1H), 7.18 (br t, J=7.6 Hz, 1H), 4.10-3.94 (m, 2H), 3.63 (br d, J=13.2 Hz, 1H), 3.48-3.38 (m, 1H), 3.22 (br dd, J=6.8, 12.0 Hz, 1H), 3.05 (br dd, J=8.0, 12.0 Hz, 1H), 1.99-1.86 (m, 2H), 1.86-1.72 (m, 2H); LCMS (ESI, M+1, M+3): m/z=449.7, 451.7.

Step B. 7-(2-chloro-7-(8-ethyl-7-fluoro-3-(methoxymethoxy)naphthalen-1-yl)-8-fluoroquinazolin-4-yl)-2-thia-1,3,7-triazaspiro[4.5]decane 2,2-dioxide: To a mixture of 7-(7-bromo-2-chloro-8-fluoroquinazolin-4-yl)-2-thia-1,3,7-triazaspiro[4.5]decane 2,2-dioxide (1.28 g, 1.0 equiv) and 2-(8-ethyl-7-fluoro-3-(methoxymethoxy)naphthalen-1-yl)-4,4,5,5-tetramethyl-1,3,2-dioxaborolane (1.02 g, 1.0 equiv) in THE (10 mL) were added CataCXium A Pd G3 (207 mg, 0.1 equiv) and K₃PO₄ (1.5 M, 3.0 equiv). The reaction was stirred at 60° C. for 6 hours. The mixture was diluted with water (10 mL) and extracted with EtOAc (2×15 mL). The combined organic layers were dried over anhydrous sodium sulfate, concentrated and purified with prep-HPLC (column: Phenomenex C18 250×5 0 mm×10 um; mobile phase: [water (NH₄HCO₃)−ACN]; B %: 43%-73%, 8 min) to afford the title compound (1.10 g, 64% yield) as light yellow solid; ¹H NMR (400 MHz, DMSO-d₆) δ=7.96 (dd, J=3.6, 8.8 Hz, 1H), 7.90 (ddd, J=3.2, 6.0, 8.8 Hz, 1H), 7.66 (t, J=2.4 Hz, 1H), 7.61-7.54 (m, 1H), 7.44 (dt, J=2.0, 9.2 Hz, 1H), 7.33-7.25 (m, 1H), 7.24-7.14 (m, 1H), 7.14-7.10 (m, 1H), 5.38-5.27 (m, 2H), 4.24-3.97 (m, 2H), 3.75-3.57 (m, 2H), 3.43 (s, 3H), 3.40-3.37 (m, 2H), 3.28 (br d, J=7.2 Hz, 2H), 2.02-1.90 (m, 2H), 1.89-1.72 (m, 2H), 0.83-0.65 (m, 3H); LCMS (ESI, M+1): m/z=604.0

Step C. ((3S,7aR)-7a-(((4-(2,2-dioxido-2-thia-1,3,7-triazaspiro[4.5]decan-7-yl)-7-(8-ethyl-7-fluoro-3-(methoxymethoxy)naphthalen-1-yl)-8-fluoroquinazolin-2-yl)oxy)methyl)hexahydro-1H-pyrrolizin-3-yl)methyl dimethylcarbamate: To a solution of ((3S,7aR)-7a-(hydroxymethyl)hexahydro-1H-pyrrolizin-3-yl)methyl dimethylcarbamate (104 mg, 1.3 equiv) in DMAc (2 mL) was added NaH (53.0 mg, 60% purity, 4.0 equiv) at 0° C. The reaction was stirred at 0° C. for 0.5 hours, and then the reaction was added 7-(2-chloro-7-(8-ethyl-7-fluoro-3-(methoxymethoxy)naphthalen-1-yl)-8-fluoroquinazolin-4-yl)-2-thia-1,3,7-triazaspiro[4.5]decane 2,2-dioxide (200 mg, 1.0 equiv). The reaction mixture was stirred at 20° C. for 3.5 hours. The mixture was quenched with H₂O (3 mL) and extracted with EtOAc (2×5 mL). The combined organic layers were dried over anhydrous sodium sulfate, concentrated and purified with prep-HPLC (column: Phenomenex C18 150×25 mm×10 um; mobile phase: [water (NH₄HCO₃)−ACN]; B %: 50%-80%, 8 min) to afford the title compound (50.0 mg, 16% yield) as white solid; LCMS (ESI, M+1): m/z=810.6.

Step D. ((3S,7aR)-7a-(((4-(2,2-dioxido-2-thia-1,3,7-triazaspiro[4.5]decan-7-yl)-7-(8-ethyl-7-fluoro-3-hydroxynaphthalen-1-yl)-8-fluoroquinazolin-2-yl)oxy)methyl)hexahydro-1H-pyrrolizin-3-yl)methyl dimethylcarbamate: To a mixture of ((3S,7aR)-7a-(((4-(2,2-dioxido-2-thia-1,3,7-triazaspiro[4.5]decan-7-yl)-7-(8-ethyl-7-fluoro-3-(methoxymethoxy)naphthalen-1-yl)-8-fluoroquinazolin-2-yl)oxy)methyl)hexahydro-1H-pyrrolizin-3-yl)methyl dimethylcarbamate (50.0 mg, 1.0 equiv) in MeCN (0.3 mL) was added HCl.dioxane (4M, 0.3 mL). The reaction was stirred at 25° C. for 1 hour. The mixture was concentrated under vacuum. The residue was diluted with water (0.5 mL). The mixture was adjusted to pH=7 with saturated NaHCO₃(1 mL) and extracted with EtOAc (2×3 mL). The organic layers were washed with brine (3 mL), dried over anhydrous sodium sulfate, concentrated and purified with prep HPLC (column: Phenomenex C18 150×25 mm×10 um; mobile phase: [water (NH₄HCO₃)−ACN]; B %: 40%-70%, 8 min) to afford the title compound (5.51 mg, 11% yield) as yellow solid; ¹H NMR (400 MHz, METHANOL-d₄) δ=8.00-7.91 (m, 1H), 7.70-7.61 (m, 1H), 7.36-7.28 (m, 1H), 7.26-7.20 (m, 2H), 6.99-6.92 (m, 1H), 4.51-4.30 (m, 3H), 4.27-4.15 (m, 2H), 4.13-4.00 (m, 1H), 3.75-3.54 (m, 3H), 3.45-3.41 (m, 1H), 3.25-3.20 (m, 1H), 3.18-3.03 (m, 1H), 2.95-2.74 (m, 6H), 2.53-2.40 (m, 2H), 2.37-2.28 (m, 1H), 2.14-1.79 (m, 12H), 0.78 (dt, J=3.2, 7.2 Hz, 3H); LCMS (ESI, M+1): m/z=766.5.

### Example 102

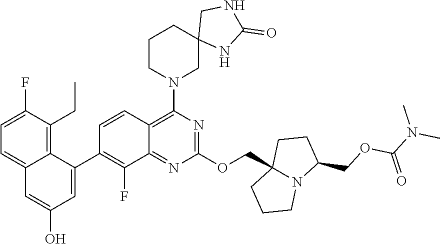 [SMILES: CCc1c(F)ccc2cc(O)cc(-c3ccc4c(N5CCCC6(CNC(=O)N6)C5)nc(OC[C@]56CCCN5[C@H](COC(=O)N(C)C)CC6)nc4c3F)c12]

### ((3S,7aR)-7a-(((7-(8-ethyl-7-fluoro-3-hydroxynaphthalen-1-yl)-8-fluoro-4-(2-oxo-1,3,7-triazaspiro[4.5]decan-7-yl)quinazolin-2-yl)oxy)methyl)hexahydro-1H-pyrrolizin-3-yl)methyl dimethylcarbamate

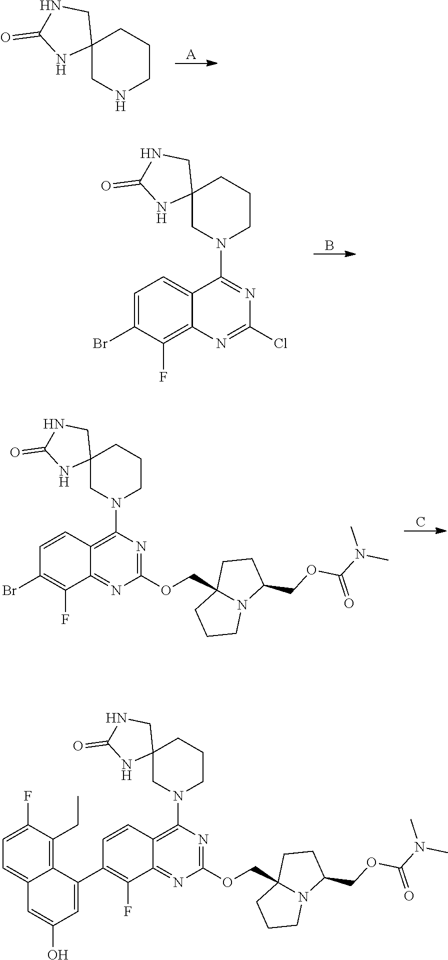

Step A. 7-(7-bromo-2-chloro-8-fluoroquinazolin-4-yl)-1,3,7-triazaspiro[4.5]decan-2-one: To a mixture of 7-bromo-2,4-dichloro-8-fluoroquinazoline (500 mg, 1.0 equiv) and DIEA (655 mg, 3.0 equiv) in DCM (5.0 mL) was added 1,3,7-triazaspiro[4.5]decan-2-one (262 mg, 1.0 equiv) at 0° C. The reaction was stirred at 25° C. for 2 hours. The mixture was filtered and the filter cake was washed with DCM (5.0 mL) to afford the title compound (600 mg, crude) as light yellow solid; LCMS (ESI, M+1, M+3): m/z=413.9, 415.9.

Step B. ((3S,7aR)-7a-(((7-bromo-8-fluoro-4-(2-oxo-1,3,7-triazaspiro[4.5]decan-7-yl)quinazolin-2-yl)oxy)methyl)hexahydro-1H-pyrrolizin-3-yl)methyl dimethylcarbamate: To a mixture of 7-(7-bromo-2-chloro-8-fluoroquinazolin-4-yl)-1,3,7-triazaspiro[4.5]decan-2-one (300 mg, 1.0 equiv) and ((3S,7aR)-7a-(hydroxymethyl)hexahydro-1H-pyrrolizin-3-yl)methyl dimethylcarbamate (263 mg, 1.5 equiv) in THF (5.0 mL) was added t-BuONa (2.0 M, 1.5 mL, 4.0 equiv) at 0° C. The reaction was stirred at 0° C. for 2 hours. The mixture was diluted with water (5.0 mL) and extracted with EtOAc (3×3.0 mL). The combined organic layers were washed with brine (5.0 mL), dried over anhydrous sodium sulfate, concentrated and purified with reversed-phase flash (0.1% FA condition) to afford the title compound (110 mg, 19% yield) as a colorless solid; LCMS (ESI, M+1, M+3): m/z=619.8, 621.8.

Step C. ((3S,7aR)-7a-(((7-(8-ethyl-7-fluoro-3-hydroxynaphthalen-1-yl)-8-fluoro-4-(2-oxo-1,3,7-triazaspiro[4.51]decan-7-yl)quinazolin-2-yl)oxy)methyl)hexahydro-1H-pyrrolizin-3-yl)methyl dimethylcarbamate: To a mixture of ((3S,7aR)-7a-(((7-bromo-8-fluoro-4-(2-oxo-1,3,7-triazaspiro[4.5]decan-7-yl)quinazolin-2-yl)oxy)methyl)hexahydro-1H-pyrrolizin-3-yl)methyl dimethylcarbamate (80.0 mg, 1.0 equiv) and 5-ethyl-6-fluoro-4-(4,4,5,5-tetramethyl-1,3,2-dioxaborolan-2-yl)naphthalen-2-ol (122 mg, 3.0 equiv) in CPME (1.0 mL) were added K₃PO₄ (1.5 M, 0.20 mL, 2.3 equiv) and cataCXium® A Pd G3 (9.39 mg, 0.1 equiv). The reaction was degassed and purged with nitrogen 3 times. The reaction was stirred at 90° C. for 2 hours. The mixture was diluted with water (2.0 mL) and extracted with EtOAc (3×2.0 mL). The combined organic layers were washed with brine (4.0 mL), dried over anhydrous sodium sulfate, concentrated and purified with column chromatography (Al₂O₃, PE/EtOAc=50/1 to 20/1, DCM/MeOH=10/1) and further purified with reversed-phase HPLC (column: Phenomenex luna C18 150×25 mm×10 um; mobile phase: [water (FA)−ACN]; B %: 16%-46%, 10 min) to afford the title compound (5.5 mg, 5.4% yield, 0.95 HCOOH) as off-white solid; ¹H NMR (400 MHz, METHANOL-d₄) δ=7.94-7.83 (m, 1H), 7.66 (dd, J=6.4, 9.2 Hz, 1H), 7.41-7.30 (m, 1H), 7.25 (d, J=2.8 Hz, 1H), 7.24-7.20 (m, 1H), 6.96 (dd, J=2.4, 6.0 Hz, 1H), 4.50-4.09 (m, 4H), 4.07-3.93 (m, 2H), 3.92-3.79 (m, 3H), 3.54-3.34 (m, 2H), 3.22-2.96 (m, 2H), 2.93-2.76 (m, 6H), 2.49-2.39 (m, 2H), 2.38-2.29 (m, 1H), 2.15-2.08 (m, 1H), 2.08-1.99 (m, 4H), 1.99-1.94 (m, 3H), 1.93-1.81 (m, 3H), 0.81-0.70 (m, 3H); LCMS (ESI, M+1): m/z=730.6.

### Example 103

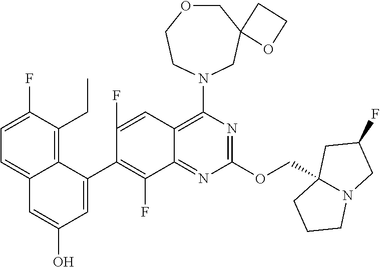 [SMILES: CCc1c(F)ccc2cc(O)cc(-c3c(F)cc4c(N5CCOCC6(CCO6)C5)nc(OC[C@@]56CCCN5C[C@H](F)C6)nc4c3F)c12]

### 4-(6,8-difluoro-2-(((2R,7aS)-2-fluorotetrahydro-1H-pyrrolizin-7a(5H)-yl)methoxy)-4-(1,6-dioxa-9-azaspiro[3.6]decan-9-yl)quinazolin-7-yl)-5-ethyl-6-fluoronaphthalen-2-ol

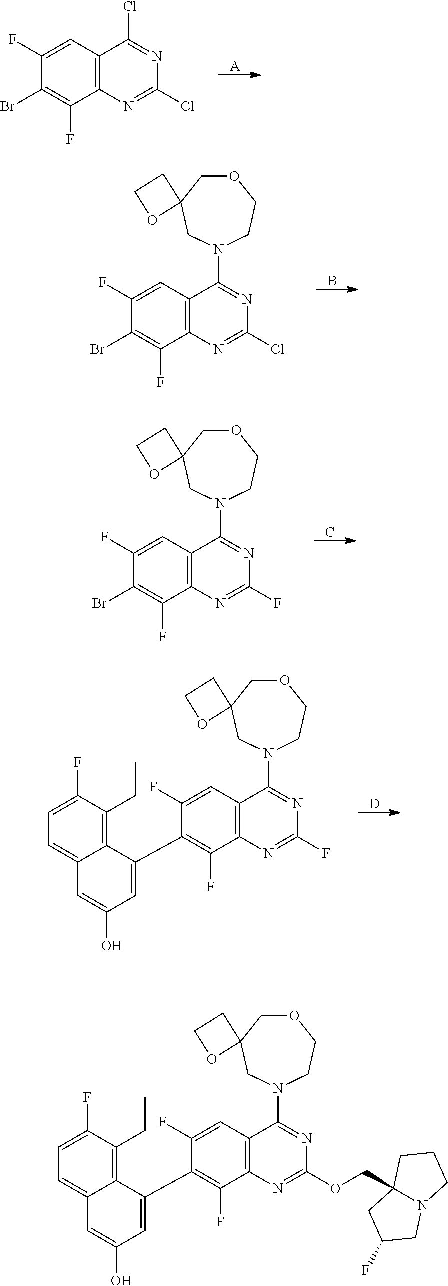

Step A. 9-(7-bromo-2-chloro-6,8-difluoroquinazolin-4-yl)-1,6-dioxa-9-azaspiro[3.6]decane: To a solution of 7-bromo-2,4-dichloro-6,8-difluoroquinazoline (100 mg, 1.0 equiv) in DMF (1.0 mL) were added DIEA (123 mg, 3.0 equiv) and 1,6-dioxa-9-azaspiro[3.6]decane (44.7 mg, 0.98 equiv). The reaction was stirred at −40° C. for 1 hour. The mixture was diluted with water (50 mL) and extracted with EtOAc (3×50 mL). The combined layers were concentrated and purified with column chromatography [SiO₂, PE/EtOAc=2/1 to 0/1] to afford the title compound (120 mg, 81% yield) as red solid; LCMS (ESI, M+1, M+3): m/z=420.0, 422.0.

Step B. 9-(7-bromo-2,6,8-trifluoroquinazolin-4-yl)-1,6-dioxa-9-azaspiro[3.6]decane: To a reaction of 9-(7-bromo-2-chloro-6,8-difluoroquinazolin-4-yl)-1,6-dioxa-9-azaspiro[3.6]decane (110 mg, 1.0 equiv) in DMSO (5.0 mL) was added KF (152 mg, 10 equiv). The reaction was stirred at 60° C. for 5 hours. The mixture was diluted with water (20 mL) and extracted with EtOAc (2×20 mL). The combined organic layers were washed with brine (30 mL), dried over anhydrous sodium sulfate, concentrated to afford the title compound (100 mg, 95% yield) as yellow solid; LCMS (ESI, M+1, M+3): m/z=404.1, 406.1.

Step C. 5-ethyl-6-fluoro-4-(2,6,8-trifluoro-4-(1,6-dioxa-9-azaspiro[3.6]decan-9-yl)quinazolin-7-yl)naphthalen-2-ol: To a mixture of 9-(7-bromo-2,6,8-trifluoroquinazolin-4-yl)-1,6-dioxa-9-azaspiro[3.6]decane (90.0 mg, 1.0 equiv) and 5-ethyl-6-fluoro-4-(4,4,5,5-tetramethyl-1,3,2-dioxaborolan-2-yl)naphthalen-2-ol (84.5 mg, 1.2 equiv) in methoxycyclopentane (2.0 mL) and H₂O (0.2 mL) were added Cs₂CO₃ (217 mg, 3.0 equiv) and CataCXium A Pd G3 (16.2 mg, 0.1 equiv). The reaction was degassed and purged with nitrogen for 3 times. The reaction was stirred at 80° C. for 1 hour. The mixture was diluted with water (40 mL) and extracted with EtOAc (2×30 mL). The combined organic layers were washed with brine (40 mL), dried over anhydrous sodium sulfate, concentrated and purified with prep-TLC [SiO₂, PE/EtOAc=1/1] to afford the title compound (60 mg, 47% yield) as red solid; LCMS (ESI, M+1): m/z=514.3.

Step D. 4-(6,8-difluoro-2-(((2R,7aS)-2-fluorohexahydro-1H-pyrrolizin-7a-yl)methoxy)-4-(1,6-dioxa-9-azaspiro[3.6]decan-9-yl)quinazolin-7-yl)-5-ethyl-6-fluoronaphthalen-2-ol: To a mixture of ((2R,7aS)-2-fluorohexahydro-1H-pyrrolizin-7a-yl)methanol (25.6 mg, 1.5 equiv) in THE (2.0 mL) was added dropwise t-BuOK (2 M, 0.16 mL) at 0° C. The reaction was stirred at 0° C. for 0.5 hours, and then 5-ethyl-6-fluoro-4-(2,6,8-trifluoro-4-(1,6-dioxa-9-azaspiro[3.6]decan-9-yl)quinazolin-7-yl)naphthalen-2-ol (55.0 mg, 1.0 equiv) in THE (2.0 mL) was added dropwise at 0° C. The reaction was stirred at 0° C. for 0.5 hours. The mixture was diluted with water (5 mL) and extracted with EtOAc (3×5 mL). The combined organic layers were washed with brine (10 mL), dried over anhydrous sodium sulfate, concentrated, and purified with prep-HPLC [column: Waters Xbridge 150×25 mm×5 um; mobile phase: [water(NH₄HCO₃)−ACN];B %: 51%-81%,8 min] to afford the title compound (5.9 mg, 8.0% yield) as white solid; ¹H NMR (400 MHz, METHANOL-d₄) δ=8.20 (br t, J=10.4 Hz, 1H), 7.70 (dd, J=6.0, 8.8 Hz, 1H), 7.35-7.22 (m, 2H), 7.00 (dd, J=2.4, 5.6 Hz, 1H), 5.42-5.21 (m, 1H), 4.83-4.43 (m, 6H), 4.33-4.19 (m, 2H), 4.17-3.95 (m, 5H), 3.84 (br dd, J=3.2, 17.6 Hz, 1H), 3.26-3.19 (m, 2H), 3.03 (br dd, J=3.6, 8.8 Hz, 1H), 2.76-2.51 (m, 3H), 2.49-2.13 (m, 4H), 2.08-1.83 (m, 3H), 0.89-0.77 (m, 3H); LCMS (ESI, M+1): m/z=653.4.

### Example 104

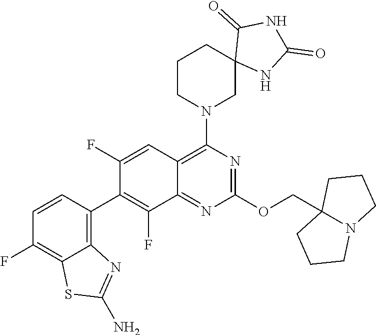 [SMILES: Nc1nc2c(-c3c(F)cc4c(N5CCCC6(C5)NC(=O)NC6=O)nc(OCC56CCCN5CCC6)nc4c3F)ccc(F)c2s1]

### 7-(7-(2-amino-7-fluorobenzo[d]thiazol-4-yl)-6,8-difluoro-2-((tetrahydro-1H-pyrrolizin-7a(5H)-yl)methoxy)quinazolin-4-yl)-1,3,7-triazaspiro[4.5]decane-2,4-dione

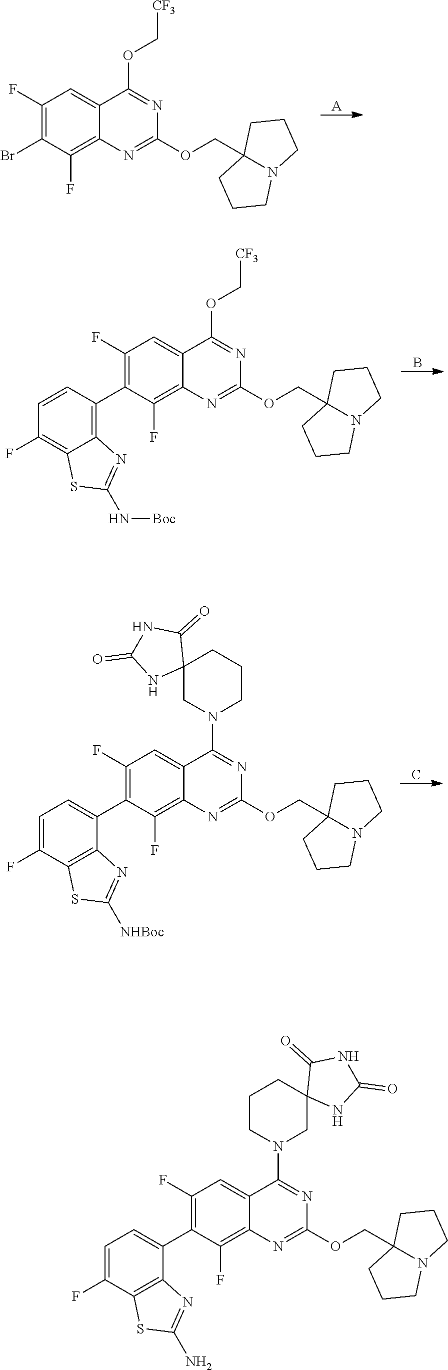

Step A. tert-butyl (4-(6,8-difluoro-2-((hexahydro-1H-pyrrolizin-7a-yl)methoxy)-4-(2,2,2-trifluoroethoxy)quinazolin-7-yl)-7-fluorobenzo[d]thiazol-2-yl)carbamate: To a mixture of 7-bromo-6,8-difluoro-2-((hexahydro-1H-pyrrolizin-7a-yl)methoxy)-4-(2,2,2-trifluoroethoxy)quinazoline (130 mg, 1.0 equiv), (2-((tert-butoxycarbonyl)amino)-7-fluorobenzo[d]thiazol-4-yl)boronic acid (109 mg, 1.3 equiv) and RuPhos (12.6 mg, 0.1 equiv) in dioxane (5.00 mL) and H₂O (1.00 mL) were added RuPhos Pd G3 (22.5 mg, 0.1 equiv) and Cs₂CO₃ (263 mg, 3.0 equiv). The reaction was degassed and purged with nitrogen 3 times. The reaction was stirred at 80° C. for 4 hours. The mixture was diluted with water (40 mL) and extracted with EtOAc (2×20 mL). The combined organic layers were washed with brine (20 mL), dried over anhydrous sodium sulfate, concentrated and purified by prep-HPLC [Unisil 3-100 C18 Ultra 150×50 mm×3 μm; A: water(FA), B:ACN;B %: 35%-65% over 7 min] to afford the title compound (45 mg, 23% yield) as white solid; LCMS (ESI, M+1): m/z=670.1.

Step B. tert-butyl (4-(4-(2,4-dioxo-1,3,7-triazaspiro[4.5]decan-7-yl)-6,8-difluoro-2-((hexahydro-1H-pyrrolizin-7a-yl)methoxy)quinazolin-7-yl)-7-fluorobenzo[d]thiazol-2-yl)carbamate: To a solution of tert-butyl (4-(6,8-difluoro-2-((hexahydro-1H-pyrrolizin-7a-yl)methoxy)-4-(2,2,2-trifluoroethoxy)quinazolin-7-yl)-7-fluorobenzo[d]thiazol-2-yl)carbamate (45.0 mg, 1.0 equiv) in DMF (0.50 mL) and acetonitrile (0.50 mL) were added 1,3,7-triazaspiro[4.5]decane-2,4-dione (22.7 mg, 2.0 equiv) and K₃PO₄ (42.8 mg, 3.0 equiv). The reaction was stirred at 40° C. for 48 hours. The mixture was filtered and purified by reversed phase flash chromatography [C18, 0.1% formic acid condition] to afford the title compound (40.0 mg, 55% yield) as yellow solid; LCMS (ESI, M+1): m/z=739.3.

Step C. 7-(7-(2-amino-7-fluorobenzo[d]thiazol-4-yl)-6,8-difluoro-2-((hexahydro-1H-pyrrolizin-7a-yl)methoxy)quinazolin-4-yl)-1,3,7-triazaspiro[4.5]decane-2,4-dione: A mixture of tert-butyl (4-(4-(2,4-dioxo-1,3,7-triazaspiro[4.5]decan-7-yl)-6,8-difluoro-2-((hexahydro-1H-pyrrolizin-7a-yl)methoxy)quinazolin-7-yl)-7-fluorobenzo[d]thiazol-2-yl)carbamate (40.0 mg, 1.0 equiv) and HCl.MeOH (4M, 1.0 mL) was stirred at 20° C. for 16 hours. The mixture was concentrated and purified by prep-HPLC [Unisil 3-100 C18 Ultra 150×50 mm×3 μm; A: water(FA), B: ACN, B %: 15%-45% over 7 min] to afford the title compound (5.04 mg, 14% yield) as white solid; ¹H NMR (400 MHz, METHANOL-d₄) δ=7.66 (br d, J=9.6 Hz, 1H), 7.36-7.27 (m, 1H), 7.05-6.96 (m, 1H), 4.61 (br s, 2H), 4.45-4.24 (m, 2H), 3.73-3.63 (m, 3H), 3.63-3.51 (m, 1H), 3.29-3.22 (m, 2H), 2.38-2.27 (m, 2H), 2.14 (br s, 9H), 1.95 (br d, J=12.8 Hz, 1H); LCMS (ESI, M+1): m/z=639.1.

### Example 105

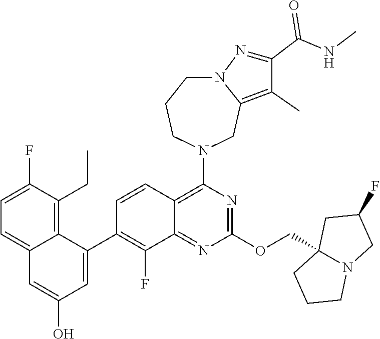 [SMILES: CCc1c(F)ccc2cc(O)cc(-c3ccc4c(N5CCCn6nc(C(=O)NC)c(C)c6C5)nc(OC[C@@]56CCCN5C[C@H](F)C6)nc4c3F)c12]

### 5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-N,3-dimethyl-5,6,7,8-tetrahydro-4H-pyrazolo[1,5-a][1,4]diazepine-2-carboxamide

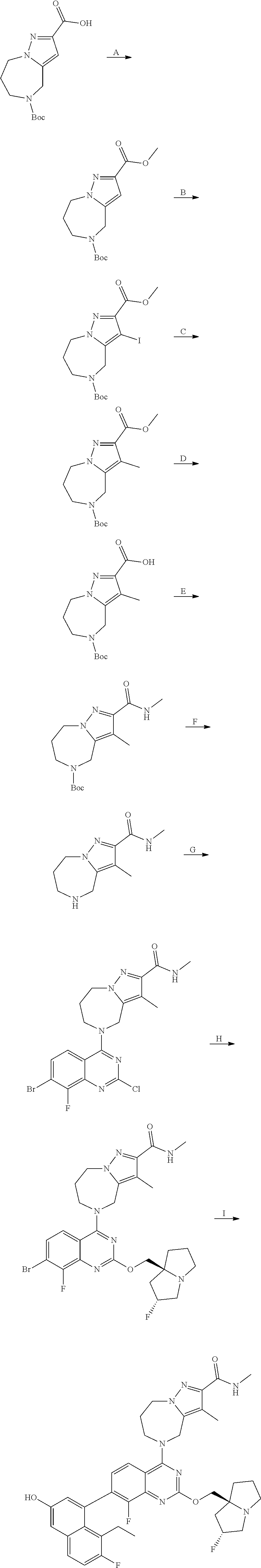

Step A. 5-tert-butyl 2-methyl 7,8-dihydro-4H-pyrazolo[1,5-a][1,4]diazepine-2,5(6H)-dicarboxylate: To a solution of 5-(tert-butoxycarbonyl)-5,6,7,8-tetrahydro-4H-pyrazolo[1,5-a][1,4]diazepine-2-carboxylic acid (5.00 g, 1.0 equiv) in MeOH (25 mL) was added diazomethyl(trimethyl)silane (2 M, 18 mL, 2.0 equiv). The reaction was stirred at 25° C. for 0.5 hours. The mixture was filtered and concentrated to afford the title compound (2.40 g, 81% yield) as yellow solid; LCMS (ESI, M+1): m/z=296.2.

Step B. 5-tert-butyl 2-methyl 3-iodo-7,8-dihydro-4H-pyrazolo[1,5-a][1,4]diazepine-2,5(6H)-dicarboxylate: To a solution of 5-tert-butyl 2-methyl 7,8-dihydro-4H-pyrazolo[1,5-a][1,4]diazepine-2,5(6H)-dicarboxylate (2.40 g, 1.0 equiv) in acetic acid (25 mL) was added NIS (3.70 g, 2.0 equiv). The reaction was stirred at 80° C. for 0.5 hours. The mixture was quenched with saturated NaHCO₃ solution (80 mL) at 0° C. and extracted with ethyl acetate (3×100 mL). The combined organic layers were washed with brine (3×50 mL), dried over anhydrous sodium sulfate, concentrated and purified with reversed phase flash chromatography [C18, 0.1% formic acid condition] to afford the title compound (2.05 g, 59% yield) as yellow solid; LCMS (ESI, M+1): m/z=422.1.

Step C. 5-tert-butyl 2-methyl 3-methyl-7,8-dihydro-4H-pyrazolo[1,5-a][1,4]diazepine-2,5(6H)-dicarboxylate: To a mixture of 5-tert-butyl 2-methyl 3-iodo-7,8-dihydro-4H-pyrazolo[1,5-a][1,4]diazepine-2,5(6H)-dicarboxylate (2.00 g, 1.0 equiv) and 2,4,6-trimethyl-1,3,5,2,4,6-trioxatriborinane (4.80 g, 50% purity, 4.0 equiv) in DMF (20 mL) were added Pd(dppf)Cl₂ (347 mg, 0.10 equiv) and K₂CO₃ (2.0 g, 3.0 equiv). The reaction was degassed and purged with N₂ for 3 times and then the reaction was stirred at 100° C. for 5 hours. The mixture was filtered, concentrated and purified with reversed phase flash chromatography [C18, 0.1% formic acid condition] to afford the title compound (1.20 g, 82% yield) as white solid; LCMS (ESI, M+1): m/z=310.2.

Step D. 5-(tert-butoxycarbonyl)-3-methyl-5,6,7,8-tetrahydro-4H-pyrazolo[1,5-a][1,4]diazepine-2-carboxylic acid: To a solution of 5-tert-butyl 2-methyl 3-methyl-7,8-dihydro-4H-pyrazolo[1,5-a][1,4]diazepine-2,5(6H)-dicarboxylate (3.0 g, 1.0 equiv) in tetrahydrofuran (15 mL), methy alcohol (7.5 mL) and H₂O (15 mL) was added LiOH.H₂O (1.20 g, 3.0 equiv). The reaction was stirred at 25° C. for 1 hour. The mixture was concentrated under reduced pressure and dissolved in water (10 mL). The mixture was adjusted to pH=8 with HCl (2.5 ml, 2 M) at 0° C. and extracted with ethyl acetate (3×100 mL). The combined organic layers were dried over anhydrous sodium sulfate and concentrated to afford the title compound (2.50 g, crude) as white solid; LCMS (ESI, M+1): m/z=296.2.

Step E. tert-butyl 3-methyl-2-(methylcarbamoyl)-7,8-dihydro-4H-pyrazolo[1,5-a][1,4]diazepine-5(6H)-carboxylate: To a mixture of 5-(tert-butoxycarbonyl)-3-methyl-5,6,7,8-tetrahydro-4H-pyrazolo[1,5-a][1,4]diazepine-2-carboxylic acid (1.00 g, 1.0 equiv) and DIEA (3.51 g, 8.0 equiv) in DMF (10 mL) were added HATU (1.93 g, 1.5 equiv) and methanamine (1.14 g, 5.0 equiv, HCl). The reaction was stirred at 20° C. for 11 hours. The mixture was diluted with water (50 mL) and extracted with ethyl acetate (4×30 mL). The combined organic layers were washed with brine (50 mL), dried over anhydrous sodium sulfate, concentrated and purified with reversed phase flash chromatography [C18, 0.1% formic acid condition] to afford the title compound (400 mg, 37% yield) as yellow solid; LCMS (ESI, M+1): m/z=309.1.

Step F. N,3-dimethyl-5,6,7,8-tetrahydro-4H-pyrazolo[1,5-a][1,4]diazepine-2-carboxamide: To a solution of tert-butyl 3-methyl-2-(methylcarbamoyl)-7,8-dihydro-4H-pyrazolo[1,5-a][1,4]diazepine-5(6H)-carboxylate (800 mg, 1.0 equiv) in acetonitrile (4 mL) was added HCl.dioxane (4 M, 8 mL). The reaction was stirred at 0° C. for 0.5 hours. The mixture was concentrated under reduced pressure and dissolved in water (5 mL). The mixture was adjusted to pH=10 with NaOH solution (10% w/w) at 0° C. and extracted with dichloromethane (4×30 mL). The combined organic layers were dried over anhydrous sodium sulfate and concentrated to afford the title compound (600 mg, crude) as yellow solid.

Step G. 5-(7-bromo-2-chloro-8-fluoroquinazolin-4-yl)-N,3-dimethyl-5,6,7,8-tetrahydro-4H-pyrazolo[1,5-a][1,4]diazepine-2-carboxamide: To a mixture of 7-bromo-2,4-dichloro-8-fluoroquinazoline (700 mg, 1.0 equiv) and DIEA (917 mg, 3.0 equiv) in dichloromethane (7 mL) was slowly added the solution of N,3-dimethyl-5,6,7,8-tetrahydro-4H-pyrazolo[1,5-a][1,4]diazepine-2-carboxamide (591 mg, 1.2 equiv) in dichloromethane (5 mL) at −40° C. The reaction was stirred at −40° C. for 0.5 hours. The mixture was diluted with water (10 mL) and extracted with dichloromethane (4×15 mL). The combined organic layers were washed with brine (20 mL), dried over anhydrous sodium sulfate, concentrated and triturated with acetonitrile (80 mL) at 20° C. for 20 minutes to afford the title compound (800 mg, 71% yield) as white solid; LCMS (ESI, M+1, M+3): m/z=466.9, 468.9.

Step H. 5-(7-bromo-8-fluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide: A mixture of 5-(7-bromo-2-chloro-8-fluoroquinazolin-4-yl)-N,3-dimethyl-5,6,7,8-tetrahydro-4H-pyrazolo[1,5-a][1,4]diazepine-2-carboxamide (800 mg, 1.0 equiv) and ((2R,7aS)-2-fluorohexahydro-1H-pyrrolizin-7a-yl)methanol (1.36 g, 5.0 equiv) was stirred at 90° C. for 24 hours under nitrogen atmosphere. The mixture was triturated with H₂O (30 mL) at 20° C. for 20 minutes to afford the title compound (800 mg, 65% yield) as yellow solid; LCMS (ESI, M+1, M+3): m/z=592.0, 594.0.

Step I. 5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide: To a mixture of 5-(7-bromo-8-fluoro-2-(((2R,7aS)-2-fluorohexahydro-1H-pyrrolizin-7a-yl)methoxy)quinazolin-4-yl)-N,3-dimethyl-5,6,7,8-tetrahydro-4H-pyrazolo[1,5-a][1,4]diazepine-2-carboxamide (200 mg, 1.0 equiv), 5-ethyl-6-fluoro-4-(4,4,5,5-tetramethyl-1,3,2-dioxaborolan-2-yl)naphthalen-2-ol (128 mg, 1.2 equiv) and K₃PO₄ (216 mg, 3.0 equiv) in methoxycyclopentane (2 mL) and H₂O (677 μL) was added CataCXium A Pd G3 (24.6 mg, 0.10 equiv). The reaction was degassed and purged with nitrogen 3 times. The reaction was stirred at 90° C. for 2 hours. The mixture was diluted with water (5 mL) and extracted with ethyl acetate (4×5 mL). The combined organic layers were washed with brine (15 mL), dried over anhydrous sodium sulfate, concentrated and purified with reversed phase flash chromatography [C18, 0.1% formic acid condition] and prep-HPLC [Phenomenex luna C18 150×25 mm×10 μm; A: water (FA), B: ACN; B %: 16%-46% over 10 min] to afford the title compound (22.6 mg, 9.3% yield, 0.3 HCOOH) as white solid; ¹H NMR (400 MHz, METHANOL-d₄) δ=7.87 (d, J=8.8 Hz, 1H), 7.67-7.63 (m, 1H), 7.35-7.30 (m, 1H), 7.25-7.19 (m, 2H), 6.93 (d, J=2.4 Hz, 1H), 5.44-5.25 (m, 1H), 5.16-5.08 (m, 1H), 5.05-4.99 (m, 1H), 4.51-4.42 (m, 2H), 4.39-4.21 (m, 4H), 3.50-3.32 (m, 3H), 3.12-3.06 (m, 1H), 2.86 (s, 3H), 2.53-2.41 (m, 2H), 2.41-2.30 (m, 6H), 2.20 (br s, 1H), 2.18-2.11 (m, 1H), 2.10-2.00 (m, 2H), 1.98-1.86 (m, 1H), 0.76 (t, J=7.2 Hz, 3H); LCMS (ESI, M+1, M+3): m/z=700.3.

### Example 106

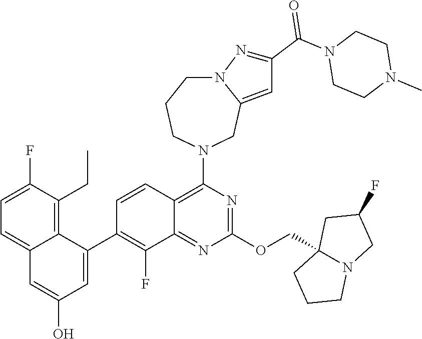 [SMILES: CCc1c(F)ccc2cc(O)cc(-c3ccc4c(N5CCCn6nc(C(=O)N7CCN(C)CC7)cc6C5)nc(OC[C@@]56CCCN5C[C@H](F)C6)nc4c3F)c12]

### (5-(7-(8-ethyl-7-fluoro-3-hydroxynaphthalen-1-yl)-8-fluoro-2-(((2R,7aS)-2-fluorotetrahydro-1H-pyrrolizin-7a(5H)-yl)methoxy)quinazolin-4-yl)-5,6,7,8-tetrahydro-4H-pyrazolo[1,5-a][1,4]diazepin-2-yl)(4-methylpiperazin-1-yl)methanone

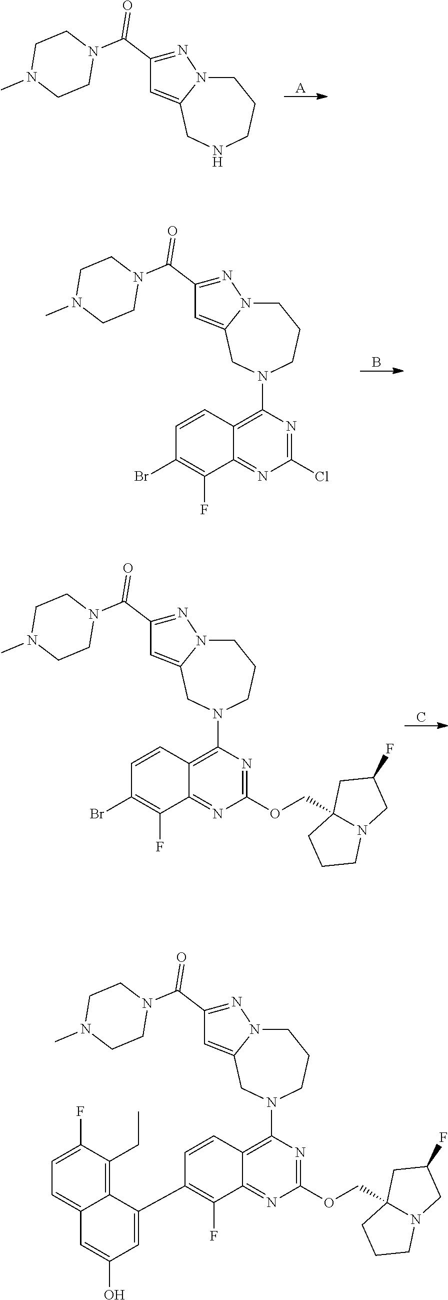

Step A. (5-(7-bromo-2-chloro-8-fluoroquinazolin-4-yl)-5,6,7,8-tetrahydro-4H-pyrazolo[1,5-a][1,4]diazepin-2-yl)(4-methylpiperazin-1-yl)methanone: To a solution of 7-bromo-2,4-dichloro-8-fluoroquinazoline (280 mg, 1.0 equiv) and (4-methylpiperazin-1-yl)(5,6,7,8-tetrahydro-4H-pyrazolo[1,5-a][1,4]diazepin-2-yl)methanone (250 mg, 1.0 equiv) in dichloromethane (1 mL) was added DIEA (367 mg, 3.0 equiv). The reaction was stirred at −40° C. for 0.5 hours. The mixture was quenched with water (10 mL) and extracted with dichloromethane (3×40 mL). The combined organic layers were dried over anhydrous sodium sulfate, concentrated, and purified with reversed phase flash [C18, 0.1% formic acid condition] to afford the title compound (380 mg, 76% yield) as white solid; LCMS (ESI, M+1, M+3): m/z=524.0, 526.0.

Step B. (5-(7-bromo-8-fluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone: A mixture of (5-(7-bromo-2-chloro-8-fluoroquinazolin-4-yl)-5,6,7,8-tetrahydro-4H-pyrazolo[1,5-a][1,4]diazepin-2-yl)(4-methylpiperazin-1-yl)methanone (380 mg, 1.0 equiv) and ((2R,7aS)-2-fluorohexahydro-1H-pyrrolizin-7a-yl)methanol (1.18 g, 10 equiv) was stirred at 90° C. for 12 hours. The mixture was quenched with water (5 mL) and extracted with dichloromethane (3×20 mL). The combined organic layers were was washed with brine (20 mL), dried over anhydrous sodium sulfate, concentrated, and purified with reversed phase flash [C18, 0.1% formic acid condition] to afford the title compound (50.0 mg, 7.2% yield) as brown solid; LCMS (ESI, M+1, M+3): m/z=645.1, 647.1.

Step C. (5-(7-(8-ethyl-7-fluoro-3-hydroxynaphthalen-1-yl)-8-fluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone: To a mixture of (5-(7-bromo-8-fluoro-2-(((2R,7aS)-2-fluorohexahydro-1H-pyrrolizin-7a-yl)methoxy)quinazolin-4-yl)-5,6,7,8-tetrahydro-4H-pyrazolo[1,5-a][1,4]diazepin-2-yl)(4-methylpiperazin-1-yl)methanone (50.0 mg, 1.0 equiv) and 5-ethyl-6-fluoro-4-(4,4,5,5-tetramethyl-1,3,2-dioxaborolan-2-yl)naphthalen-2-ol (50.0 mg, 2.0 equiv) in methoxycyclopentane (2 mL) and water (0.5 mL) were added CataCXium A Pd G3 (8.46 mg, 0.15 equiv) and Cs₂CO₃ (75.7 mg, 3.0 equiv). The reaction was degassed and purged with nitrogen 3 times. The reaction was stirred at 90° C. for 12 hours. The mixture was quenched with H₂O (5 mL) and extracted with EtOAc (3×15 mL). The combined organic layers were washed with brine (20 mL), dried over anhydrous sodium sulfate, concentrated and purified with prep-HPLC [Phenomenex luna C18 150×25 mm×10 μm; A: water (FA), B: ACN, B %: 10%-40% over 8 min] to afford the title compound (15.4 mg, 25% yield, 0.7 HCOOH) as white solid; ¹H NMR (400 MHz, METHANOL-d₄) δ=8.57-8.44 (m, 1H), 8.07-7.91 (m, 1H), 7.73-7.58 (m, 1H), 7.45-7.31 (m, 1H), 7.30-7.17 (m, 2H), 7.01-6.89 (m, 1H), 6.78-6.66 (m, 1H), 5.52-5.37 (m, 1H), 5.24-5.10 (m, 2H), 4.55-4.30 (m, 6H), 3.77 (br s, 2H), 3.72-3.37 (m, 3H), 3.27-3.21 (m, 1H), 2.60-2.35 (m, 3H), 2.35-2.32 (m, 3H), 2.32-2.26 (m, 1H), 2.22-2.13 (m, 2H), 2.09-1.98 (m, 1H), 0.84-0.69 (m, 3H); LCMS (ESI, M+1): m/z=755.2.

### Example 107

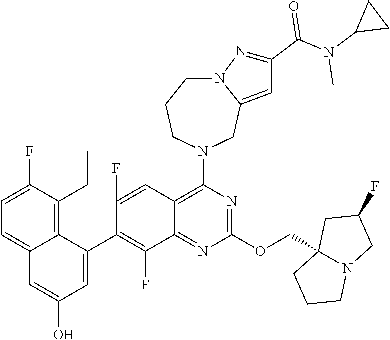 [SMILES: CCc1c(F)ccc2cc(O)cc(-c3c(F)cc4c(N5CCCn6nc(C(=O)N(C)C7CC7)cc6C5)nc(OC[C@@]56CCCN5C[C@H](F)C6)nc4c3F)c12]

### N-cyclopropyl-5-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-N-methyl-5,6,7,8-tetrahydro-4H-pyrazolo[1,5-a][1,4]diazepine-2-carboxamide

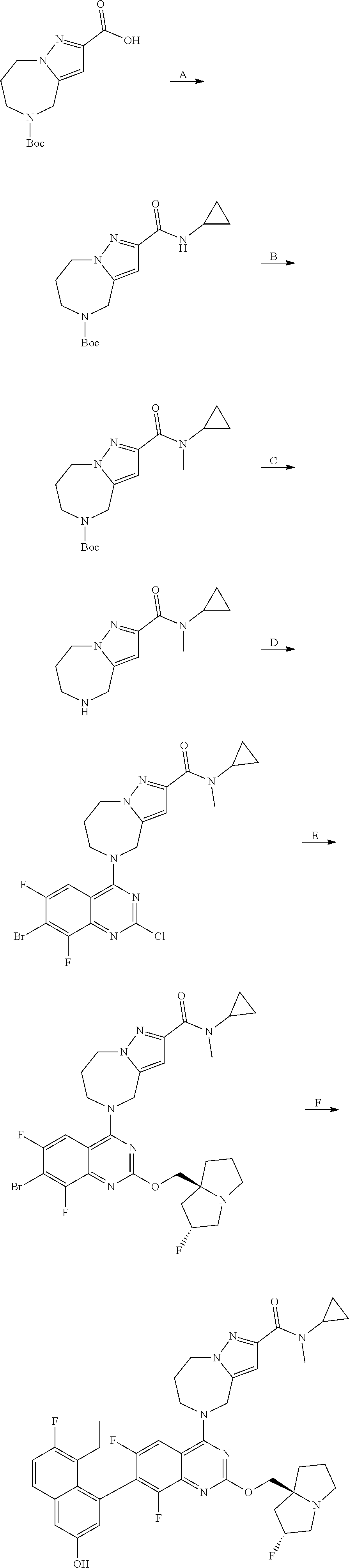

Step A. tert-butyl 2-(cyclopropylcarbamoyl)-7,8-dihydro-4H-pyrazolo[1,5-a][1,4]diazepine-5(6H)-carboxylate: To a mixture of 5-(tert-butoxycarbonyl)-5,6,7,8-tetrahydro-4H-pyrazolo[1,5-a][1,4]diazepine-2-carboxylic acid (2.00 g, 1.0 equiv) and DIPEA (2.76 g, 3.0 equiv) in DMF (25 mL) were added HATU (4.05 g, 1.5 equiv) and Cyclopropanamine (2.03 g, 5.0 equiv). The reaction was stirred at 20° C. for 2 hours. The mixture was diluted with H₂O (50 mL) and extracted with EtOAc (2×50 mL). The combined organic layers were washed with brine (50 mL), dried over anhydrous sodium sulfate, concentrated and purified with reversed phase flash [water (0.1% formic acid)/acetonitrile=1/1] to afford the title compound (2.16 g, 94% yield) as light yellow solid; LCMS (ESI, M+1): m/z=321.3.

Step B. tert-butyl 2-(cyclopropyl(methyl)carbamoyl)-7,8-dihydro-4H-pyrazolo[1,5-a][1,4]diazepine-5(6H)-carboxylate: To a solution of tert-butyl 2-(cyclopropylcarbamoyl)-7,8-dihydro-4H-pyrazolo[1,5-a][1,4]diazepine-5(6H)-carboxylate (2.1 g, 1.0 equiv) in THE (40 mL) was added NaH (524 mg, 60% purity, 2.0 equiv) at 0° C. in portions under N₂ atmosphere. The reaction was stirred between 0 and 10° C. for 0.25 hours. Then CH₃I (7.44 g, 8.0 equiv) was added dropwise at 0° C. The reaction was stirred at 25° C. for 15.75 hours. The mixture was quenched with saturated NH₄Cl aqueous (5 mL) at 0° C. The mixture was diluted with H₂O (100 mL) and extracted with EtOAc (3×30 mL). The combined organic layers were washed with brine (50 mL), dried over anhydrous sodium sulfate and concentrated to afford the title compound (2.6 g, crude) as light yellow oil; LCMS (ESI, M+1): m/z=335.1.

Step C. N-cyclopropyl-N-methyl-5,6,7,8-tetrahydro-4H-pyrazolo[1,5-a][1,4]diazepine-2-carboxamide: To a solution of tert-butyl 2-(cyclopropyl(methyl)carbamoyl)-7,8-dihydro-4H-pyrazolo[1,5-a][1,4]diazepine-5(6H)-carboxylate (2.6 g, 1.0 equiv) in MeOH (6 mL) was added HCl.dioxane (4 M, 12 mL) at 0° C. The reaction was stirred at 20° C. for 1 hour. The mixture was concentrated to afford the title compound (2.13 g, crude, HCl salt) as yellow oil; LCMS (ESI, M+1): m/z=235.1.

Step D. 5-(7-bromo-2-chloro-6,8-difluoroquinazolin-4-yl)-N-cyclopropyl-N-methyl-5,6,7,8-tetrahydro-4H-pyrazolo[1,5-a][1,4]diazepine-2-carboxamide: To a mixture of 7-bromo-2,4-dichloro-6,8-difluoroquinazoline (2.50 g, 1.0 equiv) and DIPEA (5.15 g, 5.0 equiv) in dichloromethane (30 mL) was added N-cyclopropyl-N-methyl-5,6,7,8-tetrahydro-4H-pyrazolo[1,5-a][1,4]diazepine-2-carboxamide hydrochloride (2.13 g, 0.9 equiv) at 0° C. The reaction was stirred at 20° C. for 14 hours. The mixture was diluted with H₂O (200 mL) and extracted with DCM (3×120 mL). The combined organic layers were washed with brine (200 mL), dried over anhydrous sodium sulfate and concentrated. The residue was dispersed in acetonitrile (30 mL) and stirred for 10 minutes. The mixture was filtered to afford the title compound (3.40 g, 81% yield over three steps) as yellow solid; LCMS (ESI, M+1, M+3): m/z=511.0, 513.0.

Step E. 5-(7-bromo-6,8-difluoro-2-(((2R,7aS)-2-fluorohexahydro-1H-pyrrolizin-7a-yl)methoxy)quinazolin-4-yl)-N-cyclopropyl-N-methyl-5,6,7,8-tetrahydro-4H-pyrazolo[1,5-a][1,4]diazepine-2-carboxamide: To a mixture of 5-(7-bromo-2-chloro-6,8-difluoroquinazolin-4-yl)-N-cyclopropyl-N-methyl-5,6,7,8-tetrahydro-4H-pyrazolo[1,5-a][1,4]diazepine-2-carboxamide (0.60 g, 1.0 equiv) and ((2R,7aS)-2-fluorohexahydro-1H-pyrrolizin-7a-yl)methanol (1.68 g, 9.0 equiv) in DMSO (0.5 mL) was added DIPEA (223 mg, 1.5 equiv). The reaction was stirred at 90° C. for 37 hours. The mixture was diluted with H₂O (5 mL) and extracted with EtOAc (3×10 mL). The combined organic layers were washed with brine (20 mL), dried over anhydrous sodium sulfate, concentrated, and purified with reversed phase flash [water (0.1% formic acid)/acetonitrile=2/1] to afford the title compound (453 mg, 52% yield) as yellow solid; LCMS (ESI, M+1, M+3): m/z=634.0, 636.0.

Step F. N-cyclopropyl-5-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-N-methyl-5,6,7,8-tetrahydro-4H-pyrazolo[1,5-a][1,4]diazepine-2-carboxamide: To a solution of 5-(7-bromo-6,8-difluoro-2-(((2R,7aS)-2-fluorohexahydro-1H-pyrrolizin-7a-yl)methoxy)quinazolin-4-yl)-N-cyclopropyl-N-methyl-5,6,7,8-tetrahydro-4H-pyrazolo[1,5-a][1,4]diazepine-2-carboxamide (200 mg, 1.0 equiv) and 5-ethyl-6-fluoro-4-(4,4,5,5-tetramethyl-1,3,2-dioxaborolan-2-yl)naphthalen-2-ol (110 mg, 1.1 equiv) in methoxycyclopentane (3.5 mL) were added K₃PO₄ (1.5 M in H₂O, 630 μL, 3.0 equiv) and CataCXium A Pd G3 (23.0 mg, 0.1 equiv). The reaction was degassed and purged with nitrogen 3 times. The reaction was stirred at 90° C. for 2 hours. The mixture was diluted with H₂O (10 mL) and extracted with EtOAc (3×10 mL). The combined organic layers were washed with brine (20 mL), dried over anhydrous sodium sulfate, concentrated, and purified with reversed phase flash [water (0.1% formic acid)/acetonitrile=2/1] and prep-HPLC [column: Phenomenex C18 75×30 mm×3 μm; mobile phase: water (formic acid)-acetonitrile; B %: 20%-50% over 7 minutes] to afford the title compound (75.4 mg, 32% yield, 0.3 HCOOH) as white solid; ¹H NMR (400 MHz, METHANOL-d₄) δ=7.76 (br d, J=9.6 Hz, 1H), 7.67 (dd, J=6.0, 8.8 Hz, 1H), 7.30 (d, J=2.4 Hz, 1H), 7.24 (t, J=9.6 Hz, 1H), 6.98 (d, J=2.4 Hz, 1H), 6.70 (s, 1H), 5.48-5.25 (m, 1H), 5.21-5.04 (m, 2H), 4.52 (br d, J=5.6 Hz, 2H), 4.39-4.21 (m, 4H), 3.57-3.32 (m, 4H), 3.20-3.02 (m, 4H), 2.64-2.50 (m, 1H), 2.44-2.16 (m, 6H), 2.13-1.91 (m, 3H), 0.78 (br t, J=7.2 Hz, 3H), 0.71 (br s, 2H), 0.54 (br s, 2H); ¹⁹F NMR (376 MHz, DMSO-d₆) δ=−118.8, −121.0, −124.2, −173.8; LCMS (ESI, M+1): m/z=744.1.

### Example 108

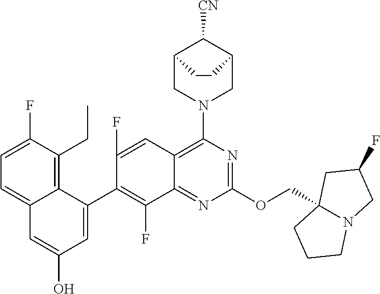 [SMILES: CCc1c(F)ccc2cc(O)cc(-c3c(F)cc4c(N5C[C@H]6CC[C@@H](C5)[C@@H]6C#N)nc(OC[C@@]56CCCN5C[C@H](F)C6)nc4c3F)c12]

### (1R,5S,8R)-3-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-azabicyclo[3.2.1]octane-8-carbonitrile

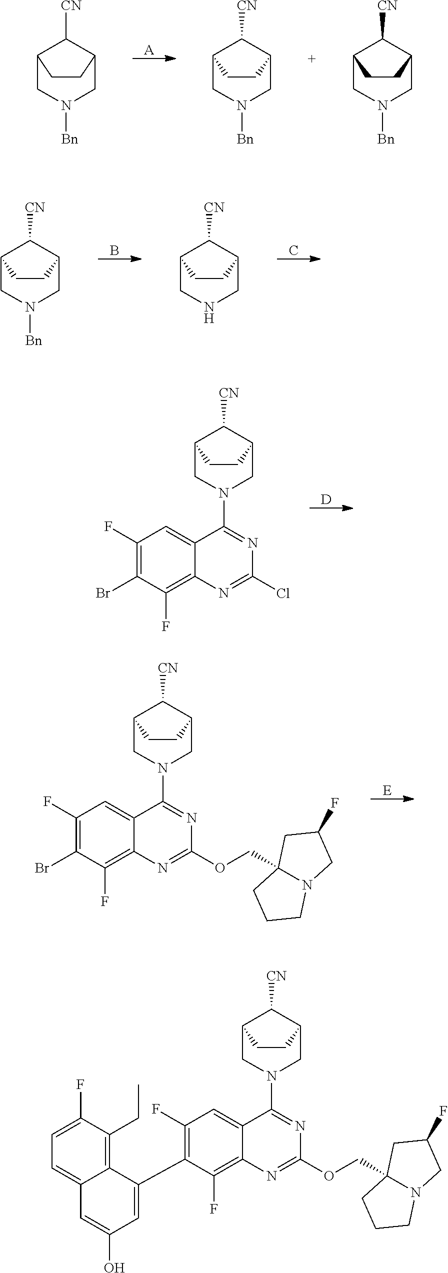

Step A. 3-benzyl-3-azabicyclo[3.2.1]octane-8-carbonitrile (4.00 g) was purified by column chromatography (SiO₂, petroleum ether/ethyl acetate 20/1 to 10/1) and SFC (column: DAICEL CHIRALPAK IG [250 mm×50 mm,10 m; A: CO₂; B: 0.1% NH₃H₂O in EtOH; B %: 10%-10% over 4.5 min) to afford two isomers.

(1R,5S,8s)-3-benzyl-3-azabicyclo[3.2.1]octane-8-carbonitrile (1.70 g, 42% yield) as white oil; ¹H NMR (400 MHz, METHANOL-d4) δ=7.23-7.08 (m, 5H), 3.36 (s, 2H), 2.59 (dd, J=4.2, 11.6 Hz, 2H), 2.52 (s, 1H), 2.32 (br s, 2H), 2.01 (s, 1H), 1.99 (s, 1H), 1.82-1.73 (m, 4H); LCMS (ESI, M+1): m/z=227.1; SFC: >97% ee, Column:Chiralpak IG-3 50×4.6 mm I.D.,3 m; mobile phase: 5% to 40% EtOH(0.05% DEA) in CO₂, flow rate:3 mL/min; detector: 220 nm, t_{R}: 0.797 min.

(1R,5S,8r)-3-benzyl-3-azabicyclo[3.2.1]octane-8-carbonitrile (1.05 g, 26% yield) as white solid; ¹H NMR (400 MHz, METHANOL-d4) δ=7.35-7.18 (m, 5H), 3.52 (s, 2H), 2.76-2.71 (m, 1H), 2.67-2.59 (m, 2H), 2.47 (d, J=11.6 Hz, 2H), 2.35 (br d, J=1.6 Hz, 2H), 1.91-1.81 (m, 2H), 1.75-1.64 (m, 2H); LCMS (ESI, M+1): m/z=227.1; SFC: >99% ee, column: Chiralpak IG-3 50×4.6 mm I.D., 3 m; mobile phase: 5% to 40% EtOH (0.05% DEA) in CO₂; flow rate:3 mL/min; detector: 220 nm, tR: 0.906 min.

Step B. (1R,5S,8s)-3-azabicyclo[3.2.1]octane-8-carbonitrile: To a solution of (1R,5S,8r)-3-benzyl-3-azabicyclo[3.2.1]octane-8-carbonitrile (500 mg, 1.0 equiv) in MeOH (10.0 mL) were added Pd/C (50 mg, 10% purity) and AcOH (265 mg, 2.0 equiv) under N₂ atmosphere. The reaction was degassed and purged with H₂ for 3 times. The reaction was stirred under H₂ (15 Psi) at 25° C. for 3 hours. The mixture was filtered and concentrated to afford the title compound (670 mg, crude, AcOH) as colourless oil; ¹H NMR (400 MHz, METHANOL-d₄) δ=3.20-3.07 (m, 4H), 2.84 (br d, J=7.2 Hz, 1H), 2.71-2.65 (m, 1H), 2.48-2.27 (m, 1H), 2.19 (br d, J=3.2 Hz, 2H), 1.92-1.69 (m, 2H).

Step C. (1R,5S,8s)-3-(7-bromo-2-chloro-6,8-difluoroquinazolin-4-yl)-3-azabicyclo[3.2.1]octane-8-carbonitrile: To a solution of 7-bromo-2,4-dichloro-6,8-difluoroquinazoline (800 mg, 1.0 equiv) and TEA (774 mg, 3 equiv) in DCM (10.0 mL) was added (1R,5S,8s)-3-azabicyclo[3.2.1]octane-8-carbonitrile (950 mg, 0.98 equiv, AcOH) at −40° C. slowly. The reaction was stirred at −40° C. for 1 hour. The mixture was quenched with water (50 mL) and extracted with DCM (2×20 mL). The combined organic layers were dried over anhydrous sodium sulfate, concentrated and purified by reversed phase flash chromatography [C18, 0.1% formic acid condition] to afford the title compound (530 mg, 48% yield) as yellow solid; LCMS (ESI, M+1, M+3): m/z=413.1, 415.1.

Step D. (1R,5S,8R)-3-(7-bromo-6,8-difluoro-2-(((2R,7aS)-2-fluorotetrahydro-1H-pyrrolizin-7a(5H)-yl)methoxy)quinazolin-4-yl)-3-azabicyclo[3.2.1]octane-8-carbonitrile: To a solution of (1R,5S,8s)-3-(7-bromo-2-chloro-6,8-difluoroquinazolin-4-yl)-3-azabicyclo[3.2.1]octane-8-carbonitrile (200 mg, 1.0 equiv) in DMSO (0.10 mL) was added ((2R,7aS)-2-fluorotetrahydro-1H-pyrrolizin-7a(5H)-yl)methanol (1.15 g, 15 equiv). The reaction was stirred at 90° C. for 16 hours. The mixture was diluted with water (20 mL) and extracted with EtOAc (2×10 mL). The combined organic layers were washed with brine (20 mL), dried over anhydrous sodium sulfate, concentrated and purified by reversed phase flash chromatography [C18, 0.1% formic acid condition] to afford the title compound (213 mg, 72% yield) as yellow solid; ¹H NMR (400 MHz, CHLOROFORM-d) δ=7.31 (br d, J=8.0 Hz, 1H), 5.36-5.18 (m, 1H), 4.36 (br d, J=12.8 Hz, 2H), 4.27-4.20 (m, 1H), 3.43-3.14 (m, 5H), 3.02-2.92 (m, 1H), 2.88-2.66 (m, 3H), 2.33-2.20 (m, 2H), 2.19-2.07 (m, 4H), 2.02-1.85 (m, 5H); LCMS (ESI, M+1, M+3): m/z=536.0, 538.0.

Step E. (1R,5S,8R)-3-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)guinazolin-4-yl)-3-azabicyclo[3.2.1]octane-8-carbonitrile: To a mixture of (1R,5S,8R)-3-(7-bromo-6,8-difluoro-2-(((2R,7aS)-2-fluorotetrahydro-1H-pyrrolizin-7a(5H)-yl)methoxy)quinazolin-4-yl)-3-azabicyclo[3.2.1]octane-8-carbonitrile (75 mg, 1.0 equiv), 5-ethyl-6-fluoro-4-(4,4,5,5-tetramethyl-1,3,2-dioxaborolan-2-yl)naphthalen-2-ol (53.0 mg, 1.2 equiv) and K₃PO₄ (3.0 equiv, 1.5 M in water) in methoxycyclopentane (2 mL) was added CataCXium-A-Pd-G3 (10.2 mg, 0.1 equiv). The reaction was degassed and purged with nitrogen 3 times. The reaction was stirred at 90° C. for 5 hours. The mixture was diluted with water (20 mL) and extracted with EtOAc (2×10 mL). The combined organic layers were dried over anhydrous sodium sulfate, concentrated and purified by reversed phase flash chromatography [C18, 0.1% formic acid condition] followed by prep-HPLC [column: YMC-Actus Triart C18 150×30 mm×7 μm; A: water(FA), B: ACN; B %: 28%-58% over 10 min] and prep-HPLC [column: YMC-Actus Triart C18 150×30 mm×7 μm; A: water(FA), B:ACN; B %: 28%-58% over 10 min] to afford the title compound (4.66 mg, 4.8% yield, HCOOH) as yellow solid; ¹H NMR (400 MHz, METHANOL-d4) δ=8.54 (br s, 1H), 7.67 (dd, J=6.0, 9.2 Hz, 1H), 7.61 (br d, J=8.8 Hz, 1H), 7.29 (d, J=2.4 Hz, 1H), 7.25 (t, J=9.2 Hz, 1H), 6.97 (d, J=2.4 Hz, 1H), 5.44-5.24 (m, 1H), 4.64-4.48 (m, 2H), 4.37-4.31 (m, 1H), 4.30-4.24 (m, 1H), 3.62-3.46 (m, 2H), 3.45-3.33 (m, 2H), 3.30-3.24 (m, 1H), 3.17 (s, 1H), 3.12-3.03 (m, 1H), 2.73 (br s, 2H), 2.62-2.50 (m, 1H), 2.46-2.24 (m, 3H), 2.23-2.13 (m, 1H), 2.11-1.99 (m, 4H), 1.98-1.87 (m, 1H), 1.87-1.74 (m, 2H), 0.80 (dt, J=2.4, 7.3 Hz, 3H); LCMS (ESI, M+1): m/z=646.4; SFC: >97% ee, Column:Chiralpak AD-3 50×4.6 mm I.D., 3 m; mobile phase: 25% IPA(0.05% DEA) in CO₂, flow rate:3 mL/min; detector: 220 nm, 4 nm, t_{R1}: 1.612 min, t_{R2}: 2.294 min.

### Example 109

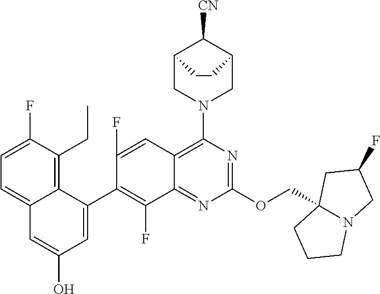 [SMILES: CCc1c(F)ccc2cc(O)cc(-c3c(F)cc4c(N5C[C@H]6CC[C@@H](C5)[C@H]6C#N)nc(OC[C@@]56CCCN5C[C@H](F)C6)nc4c3F)c12]

### (1R,5S,8S)-3-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-3-azabicyclo[3.2.1]octane-8-carbonitrile

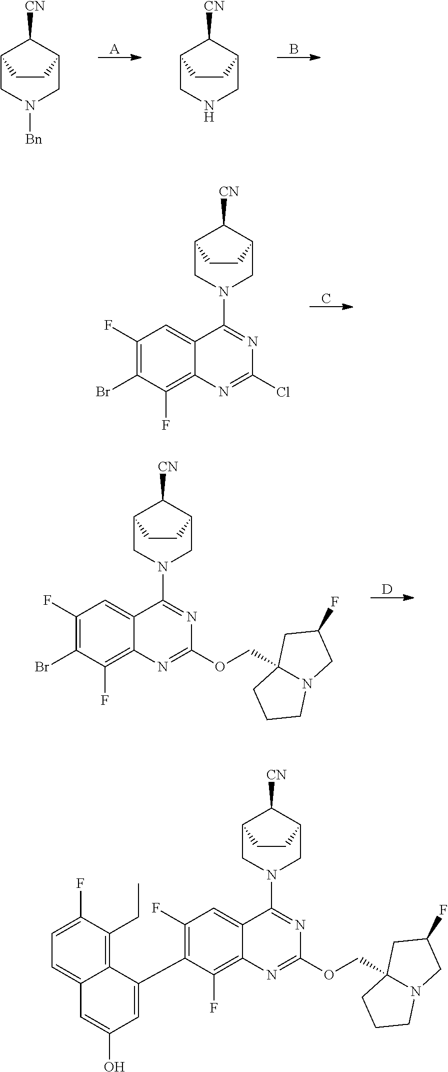

Step A. (1R,5S,8r)-3-azabicyclo[3.2.1]octane-8-carbonitrile: To a solution of (1R,5S,8r)-3-benzyl-3-azabicyclo[3.2.1]octane-8-carbonitrile (800 mg, 1.0 equiv) in MeOH (10.0 mL) were added Pd/C (80 mg, 10% purity) and AcOH (425 mg, 2.0 equiv) under N₂ atmosphere. The reaction was degassed and purged with H₂ for 3 times. The mixture was stirred under H₂ (15 Psi) at 25° C. for 3 hours. The mixture was filtered and the filtrate was concentrated under vacuum to afford the title compound (650 mg, crude, AcOH) as white solid; ¹H NMR (400 MHz, METHANOL-d4) δ=3.20-3.05 (m, 4H), 2.87-2.74 (m, 1H), 2.65 (br s, 1H), 2.49-2.30 (m, 1H), 2.20-2.05 (m, 2H), 1.90-1.68 (m, 2H).

Step B. (1R,5S,8r)-3-(7-bromo-2-chloro-6,8-difluoroquinazolin-4-yl)-3-azabicyclo[3.2.1]octane-8-carbonitrile: To a solution of 7-bromo-2,4-dichloro-6,8-difluoroquinazoline (530 mg, 1.0 equiv) and TEA (513 mg, 3.0 equiv) in DCM (10.0 mL) was slowly added (1R,5S,8r)-3-azabicyclo[3.2.1]octane-8-carbonitrile (514 mg, 0.8 equiv, AcOH) at −40° C. The reaction was stirred at −40° C. for 1 hour. The mixture was diluted with water (30 mL) and extracted with DCM (2×15 mL). The combined organic layers were dried over anhydrous sodium sulfate, and concentrated to afford the title compound (1.10 g, crude) as yellow solid; LCMS (ESI, M+1, M+3): m/z=413.1, 415.1.

Step C. (1R,5S,8S)-3-(7-bromo-6,8-difluoro-2-(((2R,7aS)-2-fluorohexahydro-1H-pyrrolizin-7a-yl)methoxy)quinazolin-4-yl)-3-azabicyclo[3.2.1]octane-8-carbonitrile: To a solution of (1R,5S,8r)-3-(7-bromo-2-chloro-6,8-difluoroquinazolin-4-yl)-3-azabicyclo[3.2.1]octane-8-carbonitrile (700 mg, 1.0 equiv) in DMSO (2.00 mL) was added ((2R,7aS)-2-fluorotetrahydro-1H-pyrrolizin-7a(5H)-yl)methanol (2.69 g, 10 equiv). The reaction was stirred at 90° C. for 16 hours. The mixture was diluted with water (100 mL) and extracted with EtOAc (2×50 mL). The combined organic layers were washed with brine (50 mL), dried over anhydrous sodium sulfate, concentrated and purified with reversed phase flash [water (0.10% FA)/acetonitrile] to afford the title compound (330 mg, 30% yield) as yellow solid; LCMS (ESI, M+1, M+3): m/z=536.1, 538.1.

Step D. (1R,5S,8S)-3-(7-(8-ethyl-7-fluoro-3-hydroxynaphthalen-1-yl)-6,8-difluoro-2-(((2R,7aS)-2-fluorohexahydro-1H-pyrrolizin-7a-yl)methoxy)quinazolin-4-yl)-3-azabicyclo[3.2.1]octane-8-carbonitrile: To a mixture of (1R,5S,8S)-3-(7-bromo-6,8-difluoro-2-(((2R,7aS)-2-fluorohexahydro-1H-pyrrolizin-7a-yl)methoxy)quinazolin-4-yl)-3-azabicyclo[3.2.1]octane-8-carbonitrile (340 mg, 1.0 equiv), 5-ethyl-6-fluoro-4-(4,4,5,5-tetramethyl-1,3,2-dioxaborolan-2-yl)naphthalen-2-ol and K₃PO₄ (1.5 M, 3.0 equiv) in methoxycyclopentane (5.00 mL) was added cataCXium-A-Pd-G3 (46.2 mg, 0.1 equiv). The reaction was degassed and purged with nitrogen 3 times. The reaction was stirred at 90° C. for 6 hours. The mixture was diluted with water (10 ml) and extracted with EtOAc (2×5 mL). The combined organic layers were dried over anhydrous sodium sulfate, concentrated and purified by prep-HPLC [column: Phenomenex luna C18 150×25 mm×10 μm; A: water(FA), B:ACN; B %: 28%-58% over 7 min) to afford the title compound (28.1 mg, 6.7% yield, HCOOH) as white solid; ¹H NMR (400 MHz, METHANOL-d₄) δ=7.76-7.62 (m, 2H), 7.30 (d, J=2.8 Hz, 1H), 7.25 (t, J=9.2 Hz, 1H), 6.98 (d, J=2.4 Hz, 1H), 5.51-5.30 (m, 1H), 4.63-4.47 (m, 2H), 4.47-4.41 (m, 1H), 4.41-4.34 (m, 1H), 3.90-3.75 (m, 2H), 3.64-3.40 (m, 3H), 3.24-3.12 (m, 1H), 3.01 (t, J=4.4 Hz, 1H), 2.66 (br s, 2H), 2.61-2.52 (m, 1H), 2.51-2.30 (m, 3H), 2.29-2.21 (m, 1H), 2.18-2.07 (m, 2H), 2.06-1.93 (m, 1H), 1.92-1.75 (m, 4H), 0.80 (dt, J=2.0, 7.4 Hz, 3H); LCMS (ESI, M+1): m/z=646.4; SFC: Column:Chiralpak AD-3 50×4.6 mm I.D.,3 μm; mobile phase:25% IPA(0.05% DEA) in CO₂; flow rate:3 mL/min; detector: 220 nm, 4 nm, t_{R1}: 1.126 min, t_{R2}: 1.461 min.

### Example 110

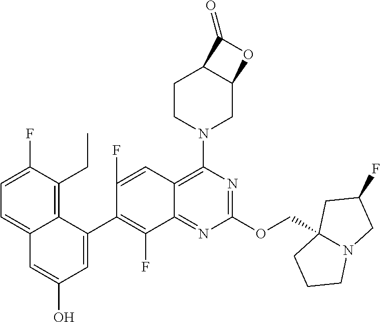 [SMILES: CCc1c(F)ccc2cc(O)cc(-c3c(F)cc4c(N5CC[C@H]6C(=O)O[C@H]6C5)nc(OC[C@@]56CCCN5C[C@H](F)C6)nc4c3F)c12]

### Cis-3-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)quinazolin-4-yl)-8-oxa-3-azabicyclo[4.2.0]octan-7-one

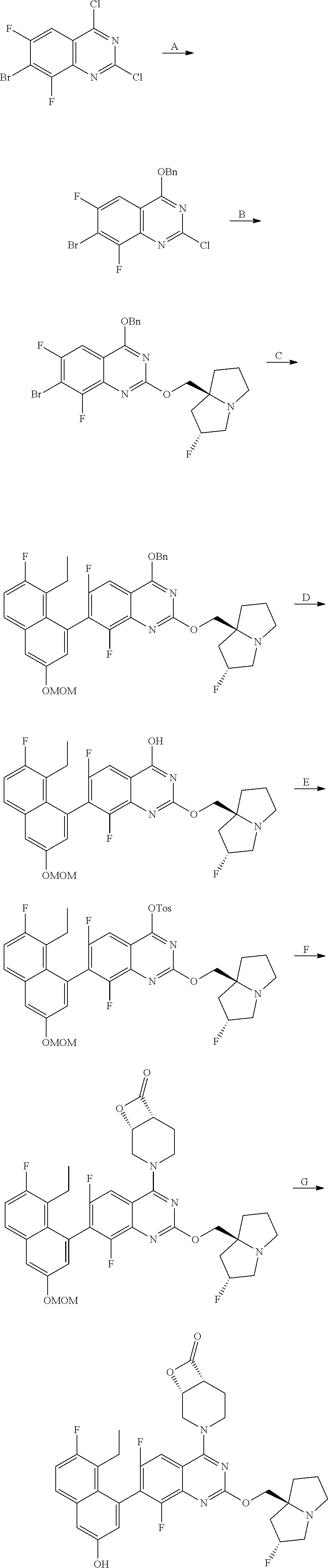

Step A: 4-(benzyloxy)-7-bromo-2-chloro-6,8-difluoroquinazoline. To a solution of NaH (700 mg, 17.5 mmol, 60% purity, 1.10 eq) in THF (150 mL) was added BnOH (2.07 g, 19.1 mmol, 1.98 mL, 1.20 eq) at 0′° C. The mixture was stirred at 0° C. for 0.5 hr. 7-bromo-2,4-dichloro-6,8-difluoroquinazoline (5.00 g, 15.9 mmol) was added in portions as solid at 0° C. The mixture was stirred at 20° C. for 1 hr. The reaction mixture was quenched by NH₄C1 (300 mL) at 0° C., and then extracted with EtOAc (200 mL). The organic layers were washed with brine (100 mL×3), dried over Na₂SO₄, filtered, and concentrated to give a residue. The crude product was triturated with PE:MTBE=2:1 (5 V) at 25° C. for 30 min to give the title compound (4.10 g, 10.33 mmol, 65.0% yield, 97.4% purity) as a gray solid. LCMS: Rt=0.682 min, m/z=387.0, M+H⁺. ¹H NMR: (400 MHz, CDCl₃): δ 6.77 (d, J=7.6 Hz, 1H), 7.53-7.41 (m, 5H), 5.66 (s, 2H).

Step B: 4-(benzyloxy)-7-bromo-6,8-difluoro-2-(((2R,7aS)-2-fluorotetrahydro-1H-pyrrolizin-7a(5H)-yl)methoxy)quinazoline. To a solution of 4-(benzyloxy)-7-bromo-2-chloro-6,8-difluoroquinazoline (3.00 g, 7.78 mol, 1.00 eq.) and ((2R,7aS)-2-fluorotetrahydro-1H-pyrrolizin-7a(5H)-yl)methanol (1.49 g, 9.34 mmol, 1.20 eq), (1.49 g, 9.34 mmol, 1.20 eq) in ACN (30 mL) was added DABCO (87.2 mg, 778 μmol, 85.5 μL, 0.10 eq) and Cs₂CO₃ (7.60 g, 23.3 mmol, 3.00 eq) at 20′° C. The mixture was stirred at 35° C. for 4 hr. The residue was poured into water (10.0 mL) and stirred for 2 min. The aqueous phase was extracted with DCM (20.0 mL×2). The combined organic phase was washed with brine (20.0 mL), dried with anhydrous Na₂SO₄, filtered, and concentrated give a residue. The residue was purified by pre-HPLC (SiO₂, column: Welch Ultimate XB-SiOH 250*50*10 um; mobile phase: [Hexane-EtOH]; gradient:1%-10% B over 15 min) to give the title compound (640 mg, 1.20 mmol, 15.4% yield, 95.3% purity) as yellow solid. LCMS: m/z=508.2, M+H⁺; LCMS: ¹HNMR: (400 MHz, CDCl₃): δ 7.58-7.37 (m, 6H), 5.61-5.54 (d, J=27 Hz, 2H), 5.38-5.24 (m, 1H), 4.39-4.27 (m, 2H), 3.35-3.25 (m, 3H), 3.02 (s, 1H), 2.32-1.97 (m, 6H)

Step C: 4-(benzyloxy)-7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline. A mixture of 4-(benzyloxy)-7-bromo-6,8-difluoro-2-(((2R,7aS)-2-fluorotetrahydro-1H-pyrrolizin-7a(5H)-yl)methoxy)quinazoline (450 mg, 745 μmol, 1.00 eq), 2-(8-ethyl-7-fluoro-3-(methoxymethoxy)naphthalen-1-yl)-4,4,5,5-tetramethyl-1,3,2-dioxaborolane (295 mg, 819 μmol, 1.10 eq), di-tert-butyl(cyclopentyl)phosphane;dichloropalladium;iron (48.58 mg, 74.5 μmol, 0.10 eq) and K₃PO₄ (1.5 M, 1.49 mL, 3.00 eq) in dioxane (5.00 mL) was degassed and purged with N₂ for 3 times. The mixture was stirred at 100° C. under N₂ atmosphere for 2 hours. The reaction mixture was quenched by H₂O (5.00 mL) at 0° C., and extracted with ethyl acetate. The combined organic layers were washed with brine (5.00 mL), dried over Na₂SO₄, filtered and concentrated to give a residue. The residue was purified by prep-HPLC (neutral condition, column: Waters Xbridge Prep OBD C18 150*40 mm*10 um; mobile phase: [water (NH₄HCO₃)−ACN];gradient:68%-98% B over 15 min). The eluent was diluted with NaHCO₃ solution (5.0 mL), and extracted with ethyl acetate (10.0 mL). The combined organic layers were washed with brine (5.00 mL), dried over Na₂SO₄, filtered and concentrated under reduced pressure to give the title compound (220 mg, 321 μmol, 43.2% yield, 80% purity) as yellow solid. LCMS: Rt=0.590 min, m/z=662.4, M+H⁺.

Step D: 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)guinazolin-4-ol. To a solution of 4-(benzyloxy)-7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazoline (190 mg, 287 μmol, 1.00 eq) in THE (2.00 mL) was added Pd/C (20.0 mg, 18.7 μmol, 10% purity) under N₂ atmosphere. The suspension was degassed and purged with H₂ for 3 times. The mixture was stirred under H₂ (45 Psi) at 20° C. for 16 hours. The reaction mixture was filtered and concentrated to give the title compound (180 mg, 238 μmol, 83.1% yield, 75.8% purity) as white solid. LCMS: Rt=0.508 min, m/z=572.4, M+H⁺. ¹HNMR (400 MHz, DMSO-d₆): δ 7.92-7.88 (m, 1H), 7.71-7.67 (m, 2H), 7.44 (t, J 8.0 Hz, 1H), 6.84 (s, 1H), 5.30 (s, 2H), 4.17-4.09 (m, 2H), 3.39 (s, 4H), 3.25-2.91 (m, 4H), 2.85-2.75 (m, 1H), 2.50-2.48 (m, 2H), 1.90-1.73 (m, 7H), 1.34 (s, 3H), 1.40-1.25 (m, 2H), 0.86-0.74 (m, 4H).

Step E: 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl 4-methylbenzenesulfonate. To a solution of 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-ol (150 mg, 262 μmol, 1.00 eq) in DCM (3.00 mL) were added TEA (106 mg, 1.05 mmol, 146 L, 4.00 eq) and TosCl (125 mg, 656 μmol, 2.50 eq). The mixture was stirred at 0° C. for 1 hr. The reaction mixture was quenched by NaHCO₃ solution (3.00 mL) at 0° C., and extracted with DCM 3.00 mL. The organic layer was washed with brine 3.00 mL, dried over Na₂SO₄, filtered, and concentrated to give compound the title compound (270 mg, crude) as yellow oil. LCMS: Rt=0.585 min, m/z=726.3, M+H⁺.

Step F: Cis-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-8-oxa-3-azabicyclo[4.2.0]octan-7-one. To a solution of 7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl 4-methylbenzenesulfonate (191 mg, 263.18 μmol, 1.00 eq) and cis-8-oxa-3-azabicyclo[4.2.0]octan-7-one (126 mg, 526.35 μmol, 2.00 eq, TFA) in DMF (2 mL) was added TEA (106 mg, 1.05 mmol, 146 μL, 4.00 eq) at 0° C. The mixture was stirred at 20° C. for 16 hrs. The mixture was purified by prep-HPLC (TFA condition, column: Phenomenex Luna C18 150*25 mm*10 um; mobile phase: [water(TFA)−ACN];gradient:33%-63% B over 9 min). The eluent was basified with sat.NaHCO₃(3 mL) at 0° C., and extracted with DCM 10 mL. The organic layer was washed with brine (3 mL), dried over Na₂SO₄, filtered, and concentrated to give the title compound (82 mg, 105.53 μmol, 40.10% yield, 87.6% purity) as white solid. LCMS: Rt=0.516 min, m/z=681.4, M+H⁺.

Step G: Cis-3-(7-(8-ethyl-7-fluoro-3-hydroxynaphthalen-1-yl)-6,8-difluoro-2-(((2R,7aS)-2-fluorotetrahydro-1H-pyrrolizin-7a(5H)-yl)methoxy)guinazolin-4-yl)-8-oxa-3-azabicyclo[4.2.0]octan-7-one. To a solution of Cis-3-(7-(8-ethyl-7-fluoro-3-(methoxymethoxy)naphthalen-1-yl)-6,8-difluoro-2-(((2R,7aS)-2-fluorotetrahydro-1H-pyrrolizin-7a(5H)-yl)methoxy)quinazolin-4-yl)-8-oxa-3-azabicyclo[4.2.0]octan-7-one (62 mg, 91.08 μmol, 1 eq) in ACN (0.6 mL) was added HCl/EtOAc (4 M, 159.40 μL, 7 eq) at 0° C. The mixture was stirred at 0° C. for 1 hr. The reaction mixture was basified with sat. NaHCO₃(1 mL) at 0° C., and extracted with DCM 5 mL. The organic layer was washed with brine 1 mL, dried over Na₂SO₄, filtered and concentrated. The residue was purified by prep-HPLC (neutral condition). The eluent was extracted with DCM (5 mL×2). The organic layer was washed with brine (3 mL), dried over Na₂SO₄, filtered, and concentrated to give the title compound (13 mg, 19.91 μmol, 21.86% yield, 97.5% purity) as off-white solid. LCMS: Rt=0.497 min, m/z=637.3, M+H⁺. ¹HNMR: EW41052-48-PIB (400 MHz, CDCl₃): δ 7.53-7.49 (m, 2H), 7.20-7.17 (m, 2H), 7.06 (s, 0.5H), 6.87 (s, 0.5H), 5.35-5.17 (m, 2H), 4.93-4.88 (m, 1H), 4.55-4.02 (m, 1H), 4.25-4.15 (m, 2H), 4.02-3.95 (m, 2H), 3.84-3.73 (m, 2H), 3.50-3.10 (m, 3H), 3.02 (s, 1H), 2.32-2.26 (m, 1H), 2.36-2.01 (m, 6H), 1.98-1.94 (m, 1H), 0.88-0.77 (m, 5H).

### Example 111

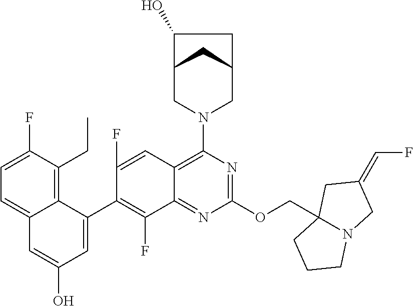 [SMILES: CCc1c(F)ccc2cc(O)cc(-c3c(F)cc4c(N5C[C@@H]6C[C@H](C5)[C@H](O)C6)nc(OCC56CCCN5C/C(=C\F)C6)nc4c3F)c12]

### (1R,5R,6R)-3-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)quinazolin-4-yl)-3-azabicyclo[3.2.1]octan-6-ol

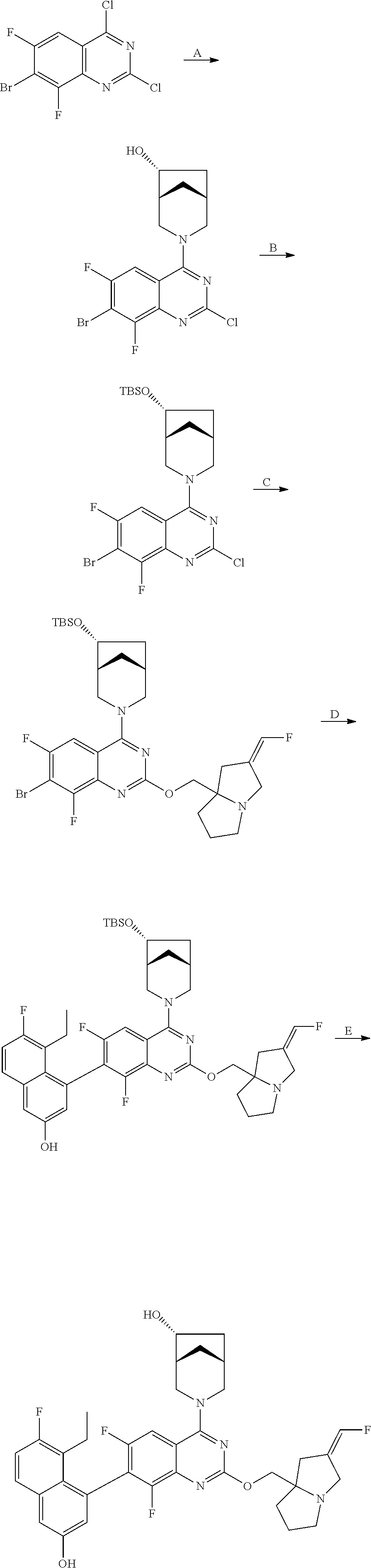

Step A. (1R,5R,6R)-3-(7-bromo-2-chloro-6,8-difluoroquinazolin-4-yl)-3-azabicyclo[3.2.1]octan-6-ol: To a solution of 7-bromo-2,4-dichloro-6,8-difluoroquinazoline (450 mg, 1.0 equiv) and DIEA (556 mg, 3.0 equiv) in DCM (5.0 mL) was added (1R,5R,6R)-3-azabicyclo[3.2.1]octan-6-ol (188 mg, 0.80 equiv, HCl salt). The reaction was stirred at −40° C. for 1 hour. The mixture was quenched with water (5.0 mL) at 25° C. and extracted with DCM (3×10 mL). The combined organic layers were dried over anhydrous sodium sulfate, concentrated and triturated with acetonitrile at 25° C. to afford the title compound (550 mg, 82% yield) as yellow solid; ¹H NMR (400 MHz, CHLOROFORM-d) δ=7.63 (dd, J=2.0, 9.2 Hz, 1H), 4.81 (br d, J=13.6 Hz, 1H), 4.38 (td, J=5.2, 10.8 Hz, 1H), 4.15 (br d, J=12.4 Hz, 1H), 3.66 (d, J=12.0 Hz, 1H), 3.50-3.34 (m, 1H), 2.34 (br d, J=10.8 Hz, 2H), 2.30-2.19 (m, 1H), 1.86-1.79 (m, 2H), 1.17 (ddd, J=2.0, 4.4, 14.2 Hz, 1H); LCMS (ESI, M+1): m/z=404.0.

Step B. 7-bromo-4-((1R,5R,6R)-6-((tert-butyldimethylsilyl)oxy)-3-azabicyclo[3.2.1]octan-3-yl)-2-chloro-6,8-difluoroquinazoline: To a solution of (1R,5R,6R)-3-(7-bromo-2-chloro-6,8-difluoroquinazolin-4-yl)-3-azabicyclo[3.2.1]octan-6-ol (530 mg, 1.0 equiv) and imidazole (268 mg, 3.0 equiv) in DMF (6 mL) were added tert-butylchlorodimethylsilane (592 mg, 3.0 equiv) and DMAP (80.0 mg, 0.50 equiv). The reaction was stirred at 25° C. for 12 hours. The mixture was quenched with water (20 mL) and extracted with ethyl acetate (3×15 mL). The combined organic layers were washed with brine (20 mL), dried over anhydrous sodium sulfate, concentrated, and purified with column chromatography (SiO₂, petroleum ether/ethyl acetate=I/O to 20/1) to afford the title compound (620 mg, 84% yield) as white solid; ¹H NMR (400 MHz, CHLOROFORM-d) δ=7.65 (dd, J=2.0, 9.2 Hz, 1H), 4.92 (td, J=2.0, 12.8 Hz, 1H), 4.48 (br d, J=11.6 Hz, 1H), 4.32-4.20 (m, 1H), 3.57 (dd, J=1.6, 11.6 Hz, 1H), 3.20 (d, J=12.8 Hz, 1H), 2.37 (br s, 1H), 2.17-2.07 (m, 2H), 1.83-1.70 (m, 2H), 1.52 (td, J=3.2, 13.6 Hz, 1H), 0.57 (s, 9H), 0.16 (d, J=2.4 Hz, 6H); LCMS (ESI, M+1): m/z=518.11.

Step C. 7-bromo-4-((1R,5R,6R)-6-((tert-butyldimethylsilyl)oxy)-3-azabicyclo[3.2.1]octan-3-yl)-6,8-difluoro-2-(((Z)-2-(fluoromethylene)tetrahydro-1H-pyrrolizin-7a(5H)-yl)methoxy)quinazoline: To a solution of 7-bromo-4-((1R,5R,6R)-6-((tert-butyldimethylsilyl)oxy)-3-azabicyclo[3.2.1]octan-3-yl)-2-chloro-6,8-difluoroquinazoline (100 mg, 1.0 equiv) and (Z)-(2-(fluoromethylene)tetrahydro-1H-pyrrolizin-7a(5H)-yl)methanol (49.5 mg, 1.5 equiv) in DMF (1.0 mL) and THE (1.0 mL) were added Cs₂CO₃ (188 mg, 3.0 equiv) and DABCO (21.6 mg, 1.0 equiv). The reaction was stirred at 25° C. for 12 hours. The mixture was diluted with water (15 mL) and extracted with EtOAc (3×10 mL). The combined organic layers were washed with brine (10 mL), dried over anhydrous sodium sulfate, concentrated, and purified with prep-TLC (petroleum ether/ethyl acetate=1/1) to afford the title compound (55.0 mg, 42% yield) as colorless gum; LCMS (ESI, M+1): m/z=653.3.

Step D. 4-(4-((1R,5R,6R)-6-((tert-butyldimethylsilyl)oxy)-3-azabicyclo[3.2.1]octan-3-yl)-6,8-difluoro-2-(((Z)-2-(fluoromethylene)tetrahydro-1H-pyrrolizin-7a(5H)-yl)methoxy)quinazolin-7-yl)-5-ethyl-6-fluoronaphthalen-2-ol: A mixture of 7-bromo-4-((1R,5R,6R)-6-((tert-butyldimethylsilyl)oxy)-3-azabicyclo[3.2.1]octan-3-yl)-6,8-difluoro-2-(((Z)-2-(fluoromethylene)tetrahydro-1H-pyrrolizin-7a(5H)-yl)methoxy)quinazoline (55.0 mg, 1.0 equiv), 5-ethyl-6-fluoro-4-(4,4,5,5-tetramethyl-1,3,2-dioxaborolan-2-yl)naphthalen-2-ol (79.8 mg, 3.0 equiv) in methoxycyclopentane (2.0 mL) were added K₃PO₄ (0.4 mL, 1.5 M, 3.0 equiv) and methanesulfonato([4-(n,n-dimethylamino)phenyl]di-t-butylphosphino)(2-amino-1,1-biphenyl-2-yl)palladium(II) (5.34 mg, 0.10 equiv). The reaction was degassed and purged with nitrogen 3 times. The reaction was stirred at 90° C. for 1 hour. The mixture was diluted with water (15 mL) and extracted with EtOAc (3×10 mL). The combined organic layers were washed with brine (10 mL), dried over anhydrous sodium sulfate, concentrated, and purified by reversed phase flash chromatography [C18, 0.1% formic acid condition] to afford the title compound (25.0 mg, 86% yield) as yellow solid; ¹H NMR (400 MHz, CHLOROFORM-d) δ=7.77 (br d, J=8.8 Hz, 1H), 7.63-7.54 (m, 1H), 7.36 (br s, 1H), 7.19 (br d, J=9.6 Hz, 1H), 7.15 (br s, 1H), 6.75-6.43 (m, 1H), 4.95-4.85 (m, 1H), 4.76-4.62 (m, 2H), 4.61-4.47 (m, 1H), 4.34-4.24 (m, 1H), 4.07-3.94 (m, 1H), 3.79 (br d, J=15.6 Hz, 1H), 3.48 (br d, J=12.8 Hz, 1H), 3.32 (br d, J=12.4 Hz, 1H), 3.12-2.88 (m, 3H), 2.65-2.55 (m, 2H), 2.51-2.33 (m, 4H), 2.31-2.21 (m, 2H), 2.20-2.11 (m, 3H), 2.03-1.95 (m, 2H), 0.82 (br s, 3H), 0.64-0.57 (m, 9H), 0.05-0.18 (m, 6H); LCMS (ESI, M+1): m/z=763.4.

Step E. (1R,5R,6R)-3-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)guinazolin-4-yl)-3-azabicyclo[3.2.1]octan-6-ol: To a solution of 4-(4-((1R,5R,6R)-6-((tert-butyldimethylsilyl)oxy)-3-azabicyclo[3.2.1]octan-3-yl)-6,8-difluoro-2-(((Z)-2-(fluoromethylene)tetrahydro-1H-pyrrolizin-7a(5H)-yl)methoxy)quinazolin-7-yl)-5-ethyl-6-fluoronaphthalen-2-ol (18.0 mg, 1.0 equiv) in MeOH (1.0 mL) was added HCl.MeOH (4 M, 1.0 mL). The reaction was stirred at 25° C. for 1 hour. The mixture was concentrated and purified with prep-HPLC [Phenomenex luna C18 150×25 mm×10 μm; A: water (FA), B: ACN; 25%-55% over 15 min] to afford the title compound (5.78 mg, 36% yield, 0.46 HCOOH) as white solid; ¹H NMR (400 MHz, METHANOL-d₄) δ=8.02 (br d, J=10.0 Hz, 1H), 7.71-7.61 (m, 1H), 7.31-7.18 (m, 2H), 6.97 (br s, 1H), 6.82-6.57 (m, 1H), 4.81-4.66 (m, 2H), 4.65-4.55 (m, 1H), 4.43-4.28 (m, 3H), 4.06-3.92 (m, 1H), 3.67-3.53 (m, 2H), 3.44 (br d, J=12.8 Hz, 1H), 2.90-2.76 (m, 2H), 2.61-2.31 (m, 4H), 2.30-2.13 (m, 3H), 2.11-1.76 (m, 5H), 1.56-1.43 (m, 1H), 0.80 (br t, J=7.4 Hz, 3H); LCMS (ESI, M+1): m/z=649.4.

### Example 112

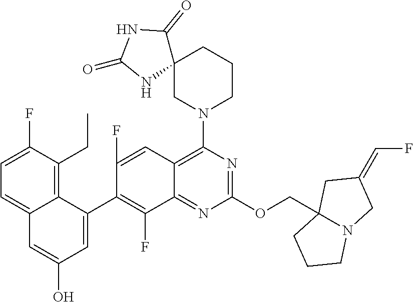 [SMILES: CCc1c(F)ccc2cc(O)cc(-c3c(F)cc4c(N5CCC[C@]6(C5)NC(=O)NC6=O)nc(OCC56CCCN5C/C(=C\F)C6)nc4c3F)c12]

### (5R)-7-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)quinazolin-4-yl)-1,3,7-triazaspiro[4.5]decane-2,4-dione

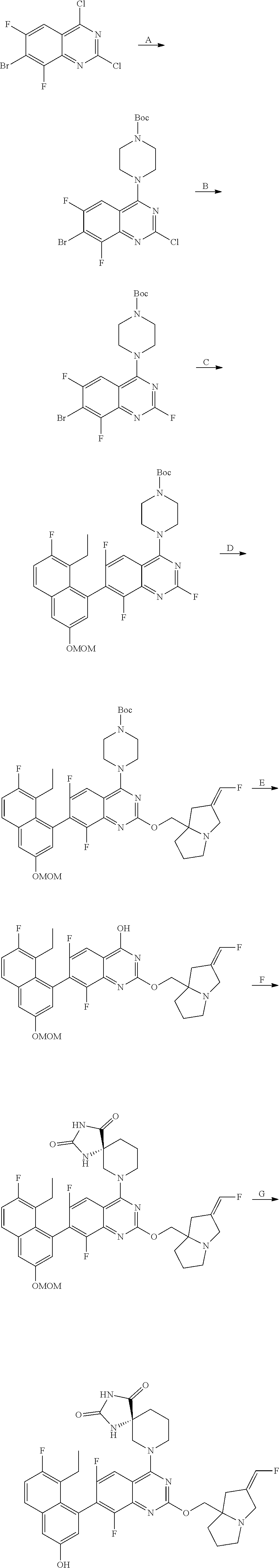

Step A. tert-butyl 4-(7-bromo-2-chloro-6,8-difluoroquinazolin-4-yl)piperazine-1-carboxylate: To a solution of 7-bromo-2,4-dichloro-6,8-difluoroquinazoline (0.90 g, 1.0 equiv) in DCM (18 mL) were added TEA (870 mg, 3.0 equiv) and tert-butyl piperazine-1-carboxylate (572 mg, 1.1 equiv) at −40° C. The reaction was stirred at −40° C. for 0.5 hours. The mixture was quenched with water (20 mL) at 0′° C. and extracted with DCM (2×10 mL). The combined organic layers were washed with brine (10 mL), dried over Na₂SO₄, concentrated and triturated with DCM (3 mL) at 25° C. to afford the title compound (1.30 g, 98% yield) as yellow solid; LCMS (ESI, M+1, M+3): m/z=463.0, 465.0.

Step B. tert-butyl 4-(7-bromo-2,6,8-trifluoroquinazolin-4-yl)piperazine-1-carboxylate: To a solution of tert-butyl 4-(7-bromo-2-chloro-6,8-difluoroquinazolin-4-yl)piperazine-1-carboxylate (1.30 g, 1.0 equiv) in DMSO (15 mL) were added KF (1.67 g, 10 equiv) and 1,4,7,10,13,16-hexaoxacyclooctadecane (75.8 mg, 0.1 equiv). The reaction was stirred at 120° C. for 2 hours. The mixture was quenched with water (100 mL) at 0° C. and extracted with DCM (2×25 mL). The combined organic layers were washed with brine (30 mL), dried over Na₂SO₄ and concentrated and purified with column chromatography (SiO₂, PE: EA=10:1 to 2:1) to afford the title compound (1.10 g, 69% yield) as yellow solid; LCMS (ESI, M+1, M+3): m/z=447.0, 449.0.

Step C. tert-butyl 4-(7-(8-ethyl-7-fluoro-3-(methoxymethoxy)naphthalen-1-yl)-2,6,8-trifluoroquinazolin-4-yl)piperazine-1-carboxylate: To a solution of tert-butyl 4-(7-bromo-2,6,8-trifluoroquinazolin-4-yl)piperazine-1-carboxylate (0.70 g, 1.0 equiv) and 2-(8-ethyl-7-fluoro-3-(methoxymethoxy)naphthalen-1-yl)-4,4,5,5-tetramethyl-1,3,2-dioxaborolane (812 mg, 1.5 equiv) in dioxane (14 mL) were added Ruphos Pd G₃ (131 mg, 0.1 equiv), Ruphos (219 mg, 0.3 equiv) and Cs₂CO₃ (1.5 M, 3.13 mL, 3.0 equiv). The reaction was degassed and purged with nitrogen for 3 times. The reaction was stirred at 60° C. for 12 hours under nitrogen atmosphere. The mixture was quenched with water (10 mL) and extracted with DCM (2×10 mL). The combined organic layers were washed with brine (10 mL), dried over Na₂SO₄, concentrated and purified with column chromatography (SiO₂, PE: EA=10:1 to 5:1) to afford the title compound (400 mg, 29% yield) as yellow solid; LCMS (ESI, M+1): m/z=601.3.

Step D. tert-butyl (Z)-4-(7-(8-ethyl-7-fluoro-3-(methoxymethoxy)naphthalen-1-yl)-6,8-difluoro-2-((2-(fluoromethylene)tetrahydro-1H-pyrrolizin-7a(5H)-yl)methoxy)quinazolin-4-yl)piperazine-1-carboxylate: To a mixture of (Z)-(2-(fluoromethylene)tetrahydro-1H-pyrrolizin-7a(5H)-yl)methanol (103 mg, 1.2 equiv) and THE (5 mL) was added portion-wise sodium hydride (59.9 mg, 3.0 equiv, 60% purity) at 0° C. The mixture was stirred at 0° C. for 10 minutes and then tert-butyl 4-(7-(8-ethyl-7-fluoro-3-(methoxymethoxy)naphthalen-1-yl)-2,6,8-trifluoroquinazolin-4-yl)piperazine-1-carboxylate (0.30 g, 1.0 equiv) in THE (2 mL) was added dropwise at 25° C. The reaction was stirred at 50° C. for 1 hour. The mixture was quenched with water (20 mL) at 0° C. and extracted with DCM (2×10 mL). The combined organic layers were washed with brine (10 mL), dried over Na₂SO₄, concentrated and purified with prep-TLC (SiO₂, DCM: MeOH=10:1) to afford the title compound (100 mg, 23% yield) as yellow solid; LCMS (ESI, M+1): m/z=752.4.

Step E. (Z)-7-(8-ethyl-7-fluoro-3-(methoxymethoxy)naphthalen-1-yl)-6,8-difluoro-2-((2-(fluoromethylene)tetrahydro-1H-pyrrolizin-7a(5H)-yl)methoxy)guinazolin-4-ol: To a mixture of tert-butyl (Z)-4-(7-(8-ethyl-7-fluoro-3-(methoxymethoxy)naphthalen-1-yl)-6,8-difluoro-2-((2-(fluoromethylene)tetrahydro-1H-pyrrolizin-7a(5H)-yl)methoxy)quinazolin-4-yl)piperazine-1-carboxylate (0.10 g, 1.0 equiv) and EtOH (10 mL) were added NaOH (26.6 mg, 5.0 equiv) and water (1 mL). The reaction was stirred at 50° C. for 5 hours. The mixture was concentrated to remove solvent. The residue was diluted with water (10 mL) and extracted with DCM (2×5 mL). The combined organic layers were washed with brine (10 mL), dried over Na₂SO₄, concentrated and purified with prep-HPLC (0.1% FA condition) to afford the title compound (68.0 mg, 73% yield) as yellow solid; LCMS (ESI, M+1): m/z=584.3.

Step F. (5R)-7-(7-(8-ethyl-7-fluoro-3-(methoxymethoxy)naphthalen-1-yl)-6,8-difluoro-2-(((Z)-2-(fluoromethylene)tetrahydro-1H-pyrrolizin-7a(5H)-yl)methoxy)quinazolin-4-yl)-1,3,7-triazaspiro[4.5]decane-2,4-dione: To a solution of (Z)-7-(8-ethyl-7-fluoro-3-(methoxymethoxy)naphthalen-1-yl)-6,8-difluoro-2-((2-(fluoromethylene)tetrahydro-1H-pyrrolizin-7a(5H)-yl)methoxy)quinazolin-4-ol (68.0 mg, 1.0 equiv) in DMSO (1 mL) were added PyBOP (182 mg, 3.0 equiv) and TEA (70.7 mg, 6.0 equiv) at 25° C. After addition, the reaction was stirred at 25° C. for 30 minutes and then (R)-1,3,7-triazaspiro[4.5]decane-2,4-dione (118 mg, 6.0 equiv) was added in portions at 25° C. The reaction was stirred at 45° C. for 16 hours. The mixture was quenched with water (10 mL) at 25° C. and extracted with EtOAc (2×5 mL). The combined organic layers were washed with brine (10 mL), dried over Na₂SO₄, concentrated and purified with prep-TLC (SiO₂, DCM: MeOH=10:1) to afford the title compound (40.0 mg, 46% yield) as yellow solid; LCMS (ESI, M+1): m/z=735.4.

Step G. (5R)-7-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)quinazolin-4-yl)-1,3,7-triazaspiro[4.5]decane-2,4-dione: To a solution of (5R)-7-(7-(8-ethyl-7-fluoro-3-(methoxymethoxy)naphthalen-1-yl)-6,8-difluoro-2-(((Z)-2-(fluoromethylene)tetrahydro-1H-pyrrolizin-7a(5H)-yl)methoxy)quinazolin-4-yl)-1,3,7-triazaspiro[4.5]decane-2,4-dione (30.0 mg, 1.0 equiv) in ACN (1.5 mL) was added HCl.dioxane (4 M, 1.5 mL, 147 equiv). The reaction was stirred at 0° C. for 1 hour. The mixture was concentrated to remove solvent at 25° C. The residue was dissolved in saturated sodium bicarbonate solution (5 mL) and extracted with EtOAc (2×5 mL). The combined organic layers were washed with brine (5 mL), dried over Na₂SO₄, concentrated and purified with prep-HPLC (neutral condition; column: Waters Xbridge 150*25 mm*Sum; mobile phase: [water(NH₄HCO₃)−ACN];gradient:44%-74% B over 9 min) to afford the title compound (8.54 mg, 30% yield) as white solid; ¹H NMR (400 MHz, METHANOL-d4) δ=7.71-7.62 (m, 2H), 7.32-7.21 (m, 2H), 6.99 (t, J=2.8 Hz, 1H), 6.77-6.50 (m, 1H), 4.39-4.20 (m, 4H), 3.84 (br d, J=14.8 Hz, 1H), 3.65 (ddd, J=4.0, 10.0, 13.6 Hz, 1H), 3.55-3.42 (m, 2H), 3.15 (td, J=5.2, 10.4 Hz, 1H), 2.80-2.65 (m, 2H), 2.63-2.50 (m, 1H), 2.44 (br d, J=14.8 Hz, 2H), 2.25-2.04 (m, 4H), 2.00-1.80 (m, 4H), 0.80 (t, J=7.6 Hz, 3H); LCMS (ESI, M+1): m/z=691.4.

### Example 113

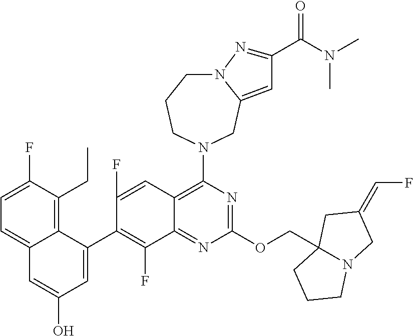 [SMILES: CCc1c(F)ccc2cc(O)cc(-c3c(F)cc4c(N5CCCn6nc(C(=O)N(C)C)cc6C5)nc(OCC56CCCN5C/C(=C\F)C6)nc4c3F)c12]

### (Z)-5-(7-(8-ethyl-7-fluoro-3-hydroxynaphthalen-1-yl)-6,8-difluoro-2-((2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

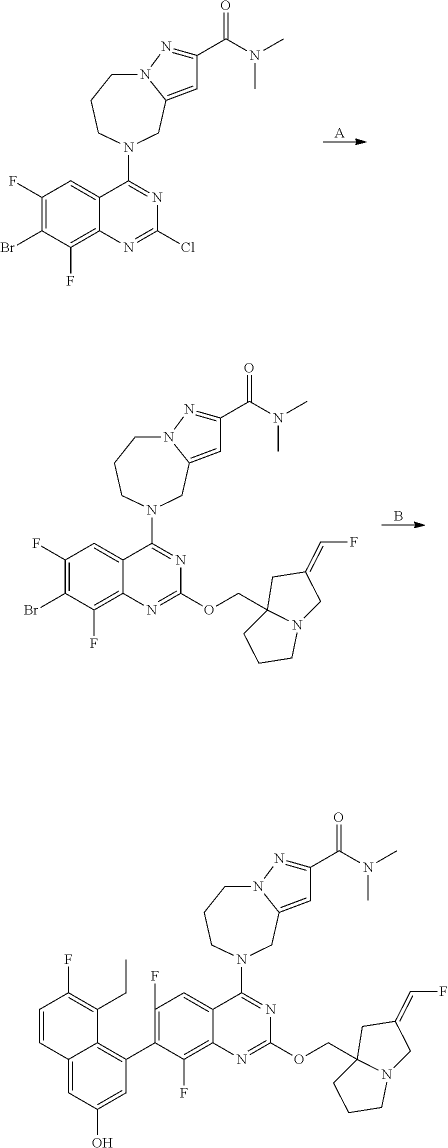

Step A. (Z)-5-(7-bromo-6,8-difluoro-2-((2-(fluoromethylene)tetrahydro-1H-pyrrolizin-7a(5H)-yl)methoxy)guinazolin-4-yl)-N,N-dimethyl-5,6,7,8-tetrahydro-4H-pyrazolo[1,5-a][1,4]diazepine-2-carboxamide: To a solution of 5-(7-bromo-2-chloro-6,8-difluoroquinazolin-4-yl)-N,N-dimethyl-5,6,7,8-tetrahydro-4H-pyrazolo[1,5-a][1,4]diazepine-2-carboxamide (150 mg, 1.0 equiv) and (Z)-(2-(fluoromethylene)tetrahydro-1H-pyrrolizin-7a(5H)-yl)methanol (63.5 mg, 1.2 equiv) in DMF (1.5 mL) and THE (1.5 mL) were added Cs₂CO₃ (302 mg, 3.0 equiv) and DABCO (34.6 mg, 1.0 equiv). The reaction was stirred at 20° C. for 12 hours. The mixture was diluted with water (5.0 mL) and extracted with EtOAc (3×5.0 mL). The combined organic layers were dried over sodium sulfate, concentrated and purified by reversed phase flash chromatography [C18, 0.1% formic acid condition] to afford the title compound (83.0 mg, 43% yield) as white solid; LCMS (ESI, M+1, M+3): m/z=620.1, 622.0.

Step B. (Z)-5-(7-(8-ethyl-7-fluoro-3-hydroxynaphthalen-1-yl)-6,8-difluoro-2-((2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a mixture of (Z)-5-(7-bromo-6,8-difluoro-2-((2-(fluoromethylene)tetrahydro-1H-pyrrolizin-7a(5H)-yl)methoxy) quinazolin-4-yl)-N,N-dimethyl-5,6,7,8-tetrahydro-4H-pyrazolo[1,5-a][1,4]diazepine-2-carboxamide (30.0 mg, 1.0 equiv), 5-ethyl-6-fluoro-4-(4,4,5,5-tetramethyl-1,3,2-dioxaborolan-2-yl)naphthalen-2-ol (22.9 mg, 1.5 equiv) and Cs₂CO₃ (1.5 M, 3.0 equiv) in dioxane (1.5 mL) were added RuPhos (4.51 mg, 0.2 equiv) and RuPhos Pd G3 (4.04 mg, 0.1 equiv). The reaction was degassed and purged with nitrogen 3 times. The reaction was stirred at 90° C. for 1 hour. The mixture was diluted with water (10 mL) and extracted with EtOAc (3×5.0 mL). The combined organic layers were dried over anhydrous sodium sulfate, concentrated and purified by prep-HPLC [Phenomenex luna C18 150×25 mm×10 μm; A: water (FA), B: ACN, B %: 24%-54% over 10 min] to afford the title compound (30.0 mg, 36% yield) as white solid; ¹H NMR (400 MHz, METHANOL-d₄) δ=7.74 (br d, J=10.0 Hz, 1H), 7.70-7.65 (m, 1H), 7.29 (d, J=2.4 Hz, 1H), 7.25 (t, J=9.2 Hz, 1H), 6.98 (d, J=2.4 Hz, 1H), 6.83-6.54 (m, 2H), 5.24-5.05 (m, 2H), 4.58-4.48 (m, 2H), 4.41-4.24 (m, 4H), 3.98 (br d, J=15.2 Hz, 1H), 3.61 (br d, J=14.4 Hz, 1H), 3.33 (s, 3H), 3.30-3.26 (m, 1H), 3.08 (s, 3H), 2.88-2.81 (m, 1H), 2.78 (br d, J=16.0 Hz, 1H), 2.62-2.46 (m, 2H), 2.39 (br s, 3H), 2.21-2.12 (m, 1H), 2.09-1.84 (m, 3H), 0.80 (t, J=7.2 Hz, 3H); LCMS (ESI, M+1): m/z=730.3.

### Example 114

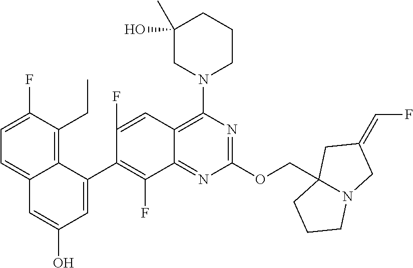 [SMILES: CCc1c(F)ccc2cc(O)cc(-c3c(F)cc4c(N5CCC[C@@](C)(O)C5)nc(OCC56CCCN5C/C(=C\F)C6)nc4c3F)c12]

### (3R)-1-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)quinazolin-4-yl)-3-methylpiperidin-3-ol

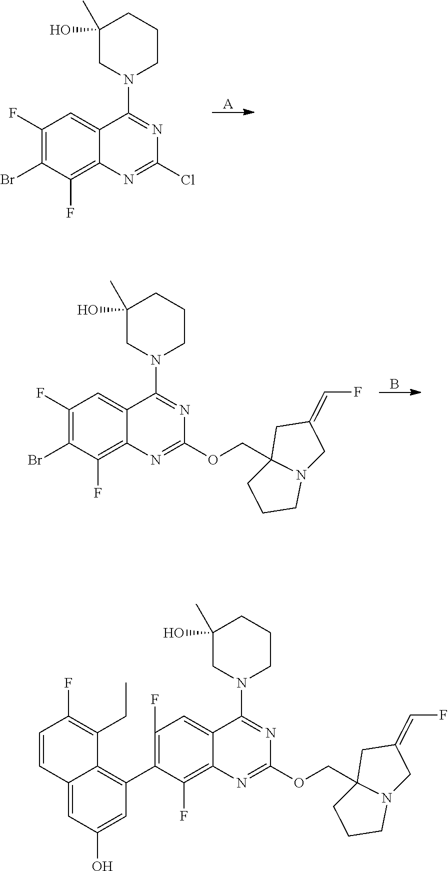

Step A. (3R)-1-(7-bromo-6,8-difluoro-2-(((Z)-2-(fluoromethylene)tetrahydro-1H-pyrrolizin-7a(5H)-yl)methoxy)quinazolin-4-yl)-3-methylpiperidin-3-ol: To a solution of (R)-1-(7-bromo-2-chloro-6,8-difluoroquinazolin-4-yl)-3-methylpiperidin-3-ol (80.0 mg, 1.0 equiv) and (Z)-(2-(fluoromethylene)tetrahydro-1H-pyrrolizin-7a(5H)-yl)methanol (38.4 mg, 1.1 equiv) in DMF (0.5 mL) and THE (0.5 mL) were added Cs₂CO₃ (199 mg, 3.0 equiv) and 1,4-diazabicyclo[2.2.2]octane (22.9 mg, 1.0 equiv). The reaction was stirred at 25° C. for 6 hours. The mixture was filtered and purified by reversed phase flash chromatography [C18, 0.1% formic acid condition] to afford the title compound (80.0 mg, 71% yield) as white oil; LCMS (ESI, M+1, M+3): m/z=527.1, 529.0.

Step B. (3R)-1-(7-(8-ethyl-7-fluoro-3-hydroxynaphthalen-1-yl)-6,8-difluoro-2-(((Z)-2-(fluoromethylene)tetrahydro-1H-pyrrolizin-7a(5H)-yl)methoxy)quinazolin-4-yl)-3-methylpiperidin-3-ol: To a mixture of (3R)-1-(7-bromo-6,8-difluoro-2-(((Z)-2-(fluoromethylene)tetrahydro-1H-pyrrolizin-7a(5H)-yl)methoxy)quinazolin-4-yl)-3-methylpiperidin-3-ol (70.0 mg, 1.0 equiv), 5-ethyl-6-fluoro-4-(4,4,5,5-tetramethyl-1,3,2-dioxaborolan-2-yl)naphthalen-2-ol (50.4 mg, 1.2 equiv) and Cs₂CO₃ (1.5 M, 265 μL, 3.0 equiv) in 1,4-dioxane (1.0 mL) was added RuPhos Pd G3 (11.1 mg, 0.1 equiv). The reaction was degassed and purged with nitrogen 3 times. The reaction was stirred at 90° C. for 1 hour. The mixture was diluted with water (2.0 mL) and extracted with EtOAc (3×5.0 mL). The combined organic layers were dried over anhydrous sodium sulfate, concentrated and purified with prep-HPLC [Phenomenex luna C18 150×25 mm×10 μm; A: water (FA); B: ACN, B %: 26%-56% over 10 min] to afford the title compound (20.5 mg, 22% yield, 0.38 HCOOH) as white solid; ¹H NMR (400 MHz, DMSO-d₆) δ=10.15-9.83 (m, 1H), 7.93-7.83 (m, 1H), 7.79 (dd, J=6.0, 9.2 Hz, 1H), 7.50-7.25 (m, 2H), 7.02 (s, 1H), 6.90-6.62 (m, 1H), 4.89-4.62 (m, 1H), 4.16-3.96 (m, 3H), 3.86 (br t, J=12.8 Hz, 1H), 3.72 (br d, J=15.2 Hz, 1H), 3.52-3.34 (m, 2H), 3.25 (br d, J=2.4 Hz, 1H), 3.07-2.97 (m, 1H), 2.67 (br s, 2H), 2.39-2.28 (m, 2H), 2.09-1.92 (m, 2H), 1.81 (br s, 2H), 1.76-1.58 (m, 4H), 1.16 (d, J=10.4 Hz, 3H), 0.75 (dt, J=3.2, 7.2 Hz, 3H); LCMS (ESI, M+1): m/z=637.3.

### Example 115

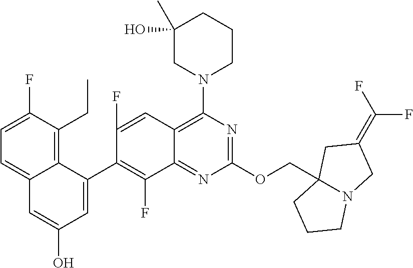 [SMILES: CCc1c(F)ccc2cc(O)cc(-c3c(F)cc4c(N5CCC[C@@](C)(O)C5)nc(OCC56CCCN5CC(=C(F)F)C6)nc4c3F)c12]

### (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-methylpiperidin-3-ol

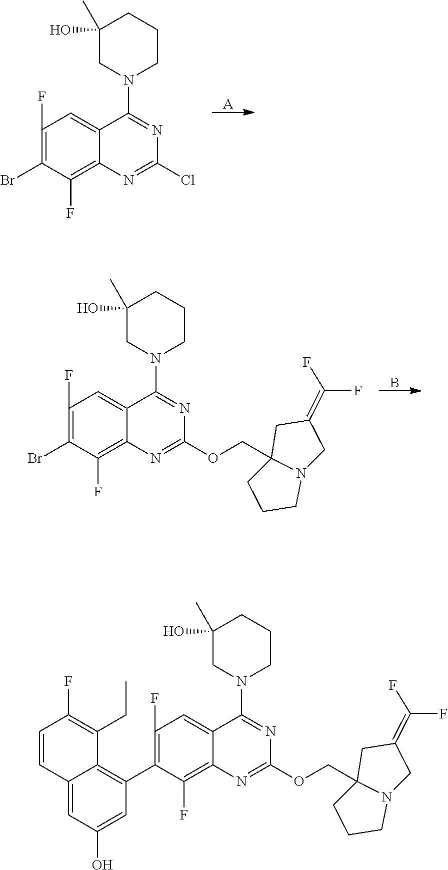

Step A. (3R)-1-(7-bromo-2-((2-(difluoromethylene)tetrahydro-1H-pyrrolizin-7a(5H)-yl)methoxy)-6,8-difluoroquinazolin-4-yl)-3-methylpiperidin-3-ol: To a solution of (R)-1-(7-bromo-2-chloro-6,8-difluoroquinazolin-4-yl)-3-methylpiperidin-3-ol (100 mg, 1.0 equiv) and (2-(difluoromethylene)tetrahydro-1H-pyrrolizin-7a(5H)-yl)methanol (72.3 mg, 1.5 equiv) in THE (0.5 mL) and DMF (0.5 mL) were added Cs₂CO₃ (249 mg, 3.0 equiv) and 1,4-diazabicyclo[2.2.2]octane (28.6 mg, 1.0 equiv). The reaction was stirred at 25° C. for 4 hours. The mixture was diluted with water (3 mL) and extracted with EtOAc (2×10 mL). The combined organic layers were dried over anhydrous sodium sulfate, concentrated and purified by reversed phase flash chromatography [C18, 0.1% formic acid condition] to afford the title compound (80 mg, 55% yield) as light yellow solid; LCMS (ESI, M+1, M+3): m/z=545.3, 547.2.

Step B. (3R)-1-(2-((2-(difluoromethylene)tetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-methylpiperidin-3-ol:

To a solution of (3R)-1-(7-bromo-2-((2-(difluoromethylene)tetrahydro-1H-pyrrolizin-7a(5H)-yl)methoxy)-6,8-difluoroquinazolin-4-yl)-3-methylpiperidin-3-ol (50.0 mg, 1.0 equiv) and 5-ethyl-6-fluoro-4-(4,4,5,5-tetramethyl-1,3,2-dioxaborolan-2-yl)naphthalen-2-ol (145 mg, 5.0 equiv) in CPME (1.0 mL) were added aqueous K₃PO₄ (0.4 mL, 1.5 M, 3.0 equiv) and APhos Pd G3 (11.7 mg, 0.20 equiv). The reaction was degassed and purged with nitrogen 3 times. The reaction was stirred at 90° C. for 4 hours. The mixture was diluted with water (5.0 mL) and extracted with EtOAc (2×8.0 mL). The combined organic layers were dried over anhydrous sodium sulfate, concentrated and purified by prep-HPLC [Welch Xtimate C18 150×25 mm×5 μm; A: water (FA); B: ACN; B %: 20%-50% over 10 min] to afford the title compound (11.3 mg, 18% yield, 0.43 HCOOH) as white solid; ¹H NMR (400 MHz, DMSO-d₆) δ=10.88-9.31 (m, 1H), 7.89 (br t, J=12.0 Hz, 1H), 7.79 (br dd, J=6.0, 8.8 Hz, 1H), 7.43-7.29 (m, 2H), 7.02 (br s, 1H), 4.88-4.60 (m, 1H), 4.21-4.01 (m, 3H), 3.86 (br t, J=12.8 Hz, 1H), 3.64 (br d, J=14.0 Hz, 1H), 3.49-3.39 (m, 3H), 2.98 (br d, J=4.8 Hz, 1H), 2.73-2.52 (m, 2H), 2.41-2.26 (m, 2H), 2.09-1.93 (m, 2H), 1.90-1.73 (m, 3H), 1.65 (br dd, J=10.4, 17.2 Hz, 3H), 1.16 (br d, J=10.4 Hz, 3H), 0.75 (br d, J=2.4 Hz, 3H); LCMS (ESI, M+1): m/z=655.4.

### Example 116

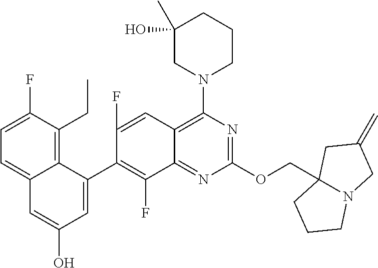 [SMILES: C=C1CN2CCCC2(COc2nc(N3CCC[C@@](C)(O)C3)c3cc(F)c(-c4cc(O)cc5ccc(F)c(CC)c45)c(F)c3n2)C1]

### (3R)-1-(7-(8-ethyl-7-fluoro-3-hydroxynaphthalen-1-yl)-6,8-difluoro-2-((2-methylenetetrahydro-1H-pyrrolizin-7a(5H)-yl)methoxy)quinazolin-4-yl)-3-methylpiperidin-3-ol

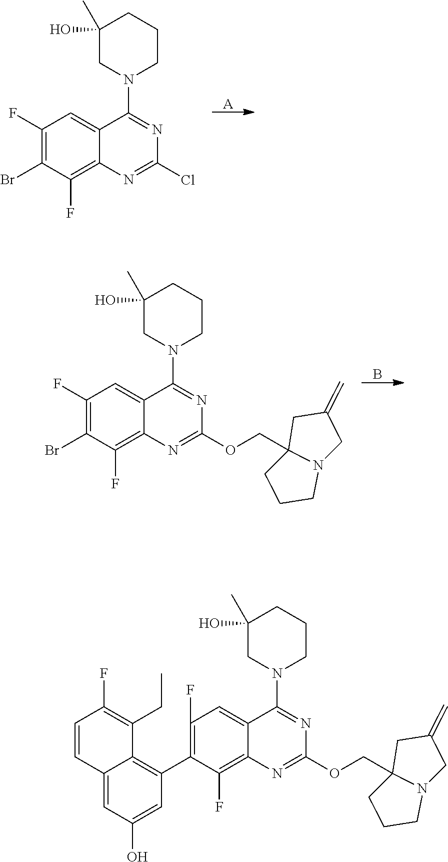

Step A. (3R)-1-(7-bromo-6,8-difluoro-2-((2-methylenetetrahydro-1H-pyrrolizin-7a(5H)-yl)methoxy)quinazolin-4-yl)-3-methylpiperidin-3-ol: To a solution of (R)-1-(7-bromo-2-chloro-6,8-difluoroquinazolin-4-yl)-3-methylpiperidin-3-ol (180 mg, 1.0 equiv), DABCO (51.4 mg, 1.0 equiv) and (2-methylenetetrahydro-1H-pyrrolizin-7a(5H)-yl)methanol (140 mg, 2.0 equiv) in THF (2 mL) and DMF (2 mL) were added Cs₂CO₃ (448 mg, 3.0 equiv) and 4 Å molecular sieve (200 mg). The reaction was stirred at 25° C. for 16 hours. The mixture was diluted with water (20 mL) and extracted with EtOAc (3×20 mL). The combined organic layers were dried over anhydrous sodium sulfate, concentrated and purified by reversed phase flash chromatography [C18, 0.1% formic acid condition] to afford the title compound (111 mg, 44% yield) as yellow solid; LCMS (ESI, M+1, M+3): m/z=509.0, 511.0.

Step B. (3R)-1-(7-(8-ethyl-7-fluoro-3-hydroxynaphthalen-1-yl)-6,8-difluoro-2-((2-methylenetetrahydro-1H-pyrrolizin-7a(5H)-yl)methoxy)quinazolin-4-yl)-3-methylpiperidin-3-ol:

To a solution of (3R)-1-(7-bromo-6,8-difluoro-2-((2-methylenetetrahydro-H-pyrrolizin-7a(5H)-yl)methoxy)quinazolin-4-yl)-3-methylpiperidin-3-1 (10.0 mg, 1.0 equiv) and 5-ethyl-6-fluoro-4-(4,4,5,5-tetramethyl-1,3,2-dioxaborolan-2-yl)naphthalen-2-ol (9.31 mg, 1.5 equiv) in 1,4-dioxane (1.0 mL) were added K₃PO₄ (1.5 M, 0.04 mL, 3.0 equiv) and CataCXium A Pd G3 (1.43 mg, 0.10 equiv). The reaction was degassed and purged with nitrogen 3 times. The reaction was stirred at 90° C. for 2 hours. The mixture was diluted with water (10 mL) and extracted with EtOAc (3×20 mL). The combined organic layers were dried over anhydrous sodium sulfate, concentrated and purified by prep-HPLC [Welch Xtimate C18 150×25 mm×5 um; A: water (FA), B: ACN; B %:18%-48%, over 9 min] to afford the title compound (11.0 mg, 14% yield) as yellow solid; ¹H NMR (400 MHz, METHANOL-d₄) δ=7.79-7.65 (m, 1H), 7.61-7.53 (m, 1H), 7.23-7.18 (m, 1H), 7.17-7.10 (m, 1H), 6.88 (s, 1H), 5.38-4.87 (m, 2H), 4.52-4.33 (m, 2H), 4.30-4.11 (m, 2H), 4.10-3.89 (m, 2H), 3.64-3.48 (m, 1H), 3.46-3.37 (m, 1H), 2.99-2.83 (m, 2H), 2.65-2.54 (m, 1H), 2.49-2.41 (m, 1H), 2.37-2.29 (m, 1H), 2.24-2.15 (m, 1H), 2.00 (br s, 2H), 1.78-1.71 (m, 2H), 1.69-1.61 (m, 2H), 1.60-1.54 (m, 1H), 1.18 (br d, J=11.6 Hz, 3H), 1.06-0.92 (m, 1H), 0.77-0.65 (m, 3H); LCMS (ESI, M+1): m/z=619.5.

### Example 117A

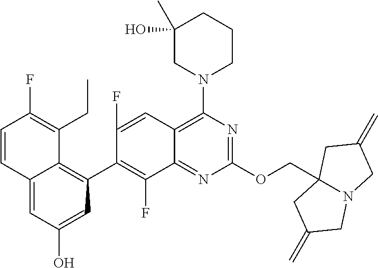

### (3R)-1-(2-((2,6-dimethylenetetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-methylpiperidin-3-ol

### Example 117B

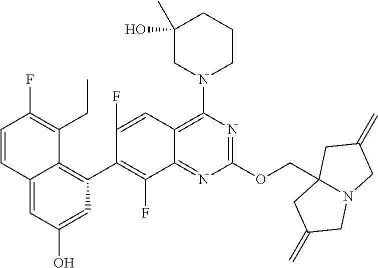

### (3R)-1-(2-((2,6-dimethylenetetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-methylpiperidin-3-ol

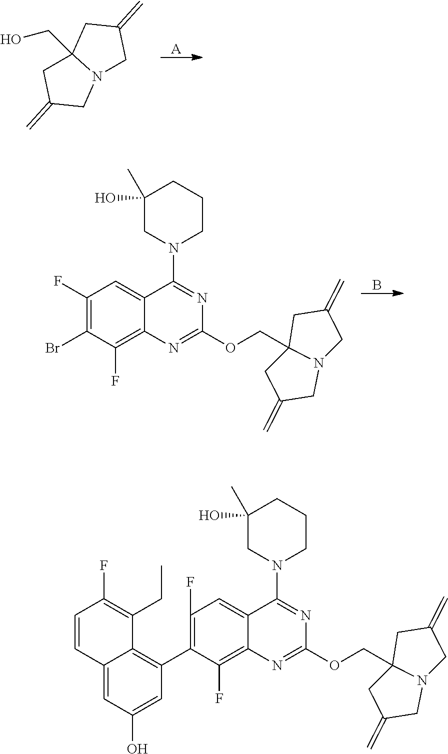

Step A. (R)-1-(7-bromo-2-((2,6-dimethylenetetrahydro-1H-pyrrolizin-7a(5H)-yl)methoxy)-6,8-difluoroquinazolin-4-yl)-3-methylpiperidin-3-ol: A mixture of (R)-1-(7-bromo-2-chloro-6,8-difluoroquinazolin-4-yl)-3-methylpiperidin-3-ol (120 mg, 1.0 equiv), (2,6-dimethylenetetrahydro-1H-pyrrolizin-7a(5H)-yl)methanol (75.7 mg, 1.5 equiv) and DABCO (34.3 mg, 1.0 equiv) in THE (2.0 mL) and DMF (2.0 mL) was added Cs₂CO₃ (299 mg, 3.0 equiv). The reaction was stirred at 20° C. for 12 hours. The mixture was diluted with water (10 mL) and extracted with EtOAc (3×15 mL). The combined organic layers were dried over anhydrous sodium sulfate, concentrated and purified by reversed phase flash chromatography (C18, 0.1% formic acid condition) to afford the title compound (120 mg, 69% yield) as yellow solid; LCMS (ESI, M+1, M+3): m/z=521.2, 523.2.

Step B. (3R)-1-(2-((2,6-dimethylenetetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-methylpiperidin-3-ol: A mixture of (R)-1-(7-bromo-2-((2,6-dimethylenetetrahydro-1H-pyrrolizin-7a(5H)-yl)methoxy)-6,8-difluoroquinazolin-4-yl)-3-methylpiperidin-3-ol (55.0 mg, 1.0 equiv), 5-ethyl-6-fluoro-4-(4,4,5,5-tetramethyl-1,3,2-dioxaborolan-2-yl)naphthalen-2-ol (50.0 mg, 1.5 equiv) and Cs₂CO₃ (1.5 M, 211 μL, 3.0 equiv) in dioxane (2.2 mL) were added RuPhos (14.8 mg, 0.3 equiv) and RuPhos Pd G3 (8.82 mg, 0.1 equiv). The reaction was degassed and purged with nitrogen 3 times. The reaction was stirred at 90° C. for 2.5 hours. The mixture was diluted with water (5.0 mL) and extracted with EtOAc (3×5 mL). The combined organic layers were dried over anhydrous sodium sulfate, concentrated and purified by prep-HPLC [Phenomenex luna C18 150×25 mm×10 μm; A:water (FA), B: ACN; 26%-56% over 10 min] and then further purified by SFC {condition: column: DAICEL CHIRALPAK IC(250 mm×30 mm×10 um); mobile phase: [CO₂-MeOH(0.1% NH₃H₂O)]; B %:45%, isocratic elution mode} to give two peaks as the title compound.

Example 117A (21.2 mg, 24.3% yield) as white solid; ¹H NMR (400 MHz, METHANOL-d₄) δ=7.80 (d, J=9.6 Hz, 1H), 7.67 (dd, J=6.0, 8.8 Hz, 1H), 7.28 (d, J=2.4 Hz, 1H), 7.24 (t, J=9.4 Hz, 1H), 6.98 (d, J=2.0 Hz, 1H), 5.01 (br d, J=6.6 Hz, 4H), 4.32 (s, 2H), 4.26-4.15 (m, 1H), 4.05 (br d, J=13.2 Hz, 1H), 3.76 (br d, J=14.6 Hz, 2H), 3.46-3.37 (m, 2H), 3.35 (br s, 2H), 2.80 (br d, J=16.4 Hz, 2H), 2.58 (br d, J=16.4 Hz, 3H), 2.50-2.36 (m, 1H), 2.14 (br d, J=13.0 Hz, 1H), 1.90-1.67 (m, 3H), 1.29 (s, 3H), 0.82 (t, J=7.2 Hz, 3H); LCMS (ESI, M+1): m/z=631.3; SFC: t_{R}=1.584, de>99.9%, Column: Chiralpak IC-3 50×4.6 mm I.D., 3 um, Mobile phase: Phase A for CO₂, and Phase B for MeOH (0.05% DEA); Isocratic elution: 30% B in A; Flow rate: 3 mL/min; Detector: PDA; Column Temp: 35C;Back Pressure: 100 Bar.

Example 117B (18.9 mg, 21.3% yield) as yellow solid; ¹H NMR (400 MHz, METHANOL-d₄) δ=7.77 (br d, J=9.6 Hz, 1H), 7.67 (dd, J=6.0, 8.8 Hz, 1H), 7.29 (d, J=2.4 Hz, 1H), 7.24 (t, J=9.6 Hz, 1H), 6.98 (d, J=2.0 Hz, 1H), 5.01 (br d, J=7.6 Hz, 4H), 4.32 (s, 2H), 4.28-4.20 (m, 1H), 4.05 (br d, J=13.2 Hz, 1H), 3.76 (br d, J=14.8 Hz, 2H), 3.50 (d, J=13.2 Hz, 1H), 3.44-3.38 (m, 1H), 3.35 (br s, 2H), 2.80 (br dd, J=3.2, 16.4 Hz, 2H), 2.58 (br d, J=16.4 Hz, 3H), 2.48-2.33 (m, 1H), 2.20-2.05 (m, 1H), 1.87-1.81 (m, 1H), 1.79-1.70 (m, 2H), 1.28-1.23 (m, 3H), 0.81 (t, J=7.6 Hz, 3H); LCMS (ESI, M+1): m/z=631.3; SFC: t_{R}=2.215, de=95.1% Column: Chiralpak IC-3 50×4.6 mm I.D., 3 um, Mobile phase: Phase A for CO₂, and Phase B for MeOH (0.05% DEA); Isocratic elution: 30% B in A; Flow rate: 3 mL/min; Detector: PDA; Column Temp: 35° C.; Back Pressure: 100 Bar.

### Example 118

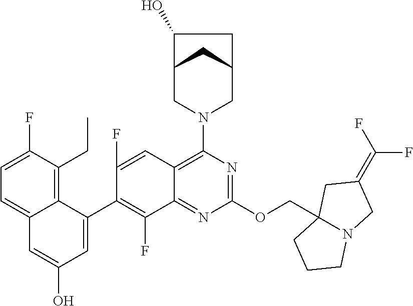 [SMILES: CCc1c(F)ccc2cc(O)cc(-c3c(F)cc4c(N5C[C@@H]6C[C@H](C5)[C@H](O)C6)nc(OCC56CCCN5CC(=C(F)F)C6)nc4c3F)c12]

### (1R,5R,6R)-3-(2-((2-(difluoromethylene)tetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-azabicyclo[3.2.1]octan-6-ol

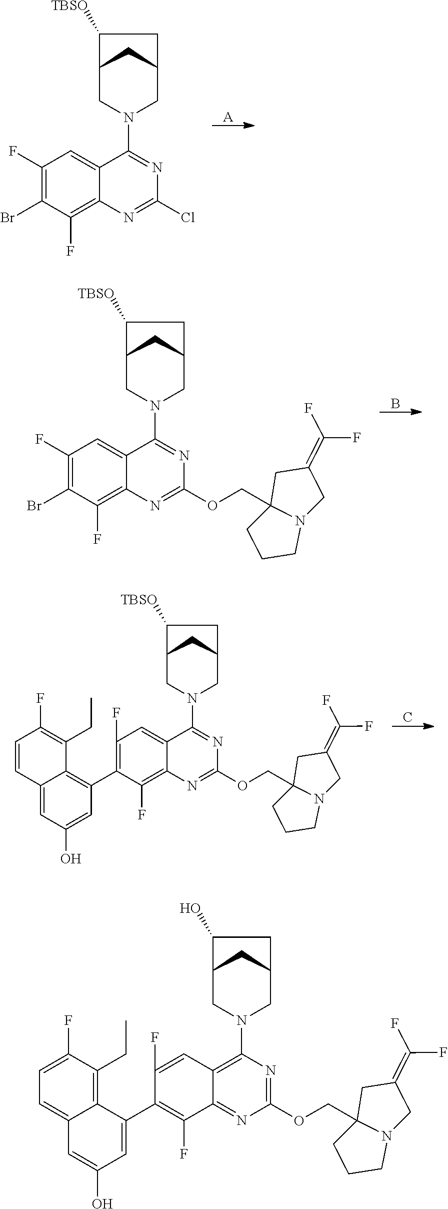

Step A. 7-bromo-4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-6,8-difluoroquinazoline: To a solution of 7-bromo-4-((1R,5R,6R)-6-((tert-butyldimethylsilyl)oxy)-3-azabicyclo[3.2.1]octan-3-yl)-2-chloro-6,8-difluoroquinazoline (100 mg, 1.0 equiv) and (2-(difluoromethylene)tetrahydro-1H-pyrrolizin-7a(5H)-yl)methanol (43.8 mg, 1.2 equiv) in DMF (1.0 mL) and THE (1.0 mL) were added Cs₂CO₃ (188 mg, 3.0 equiv) and DABCO (21.6 mg, 1.0 equiv). The reaction was stirred at 25° C. for 12 hours. The mixture was diluted with water (15 mL) and extracted with EtOAc (3×10 mL). The combined organic layers were washed with brine (10 mL), dried over anhydrous sodium sulfate, concentrated and purified with prep-TLC (petroleum ether/ethyl acetate=2/1) to afford the title compound (73.0 mg, 54% yield) as colorless gum; LCMS (ESI, M+1, M+3): m/z=671.3, 673.2.

Step B. 4-(4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-6,8-difluoroquinazolin-7-yl)-5-ethyl-6-fluoronaphthalen-2-ol: A mixture of 7-bromo-4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-6,8-difluoroquinazoline (58.0 mg, 1.0 equiv), 5-ethyl-6-fluoro-4-(4,4,5,5-tetramethyl-1,3,2-dioxaborolan-2-yl)naphthalen-2-ol (81.9 mg, 3.0 equiv) and aqueous K₃PO₄ (0.2 mL, 1.5 M, 3.0 equiv) in methoxycyclopentane (1.0 mL) was added APhos Pd G3 (5.48 mg, 0.10 equiv). The reaction was degassed and purged with nitrogen 3 times. The reaction was stirred at 90° C. for 1 hour. The mixture was diluted with water (15 mL) and extracted with EtOAc (3×10 mL). The combined organic layers were washed with brine (15 mL), dried over anhydrous sodium sulfate, concentrated and purified by reversed phase flash chromatography (C18, 0.1% formic acid condition) to afford the title compound (27.0 mg, 37% yield) as white solid; LCMS (ESI, M+1): m/z=781.4.

Step C. (1R,5R,6R)-3-(2-((2-(difluoromethylene)tetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-azabicyclo[3.2.1]octan-6-ol: To a solution of 4-(4-((1R,5R,6R)-6-((tert-butyldimethylsilyl)oxy)-3-azabicyclo[3.2.1]octan-3-yl)-2-((2-(difluoromethylene)tetrahydro-1H-pyrrolizin-7a(5H)-yl)methoxy)-6,8-difluoroquinazolin-7-yl)-5-ethyl-6-fluoronaphthalen-2-ol (24.0 mg, 1.0 equiv) in MeOH (1.0 mL) was added HCl.MeOH (4 M, 1.0 mL). The reaction was stirred at 25° C. for 1 hour. The mixture was concentrated to remove the organic solvent. The residue was dissolved in water (1 mL) and the pH was adjusted to 7 with saturated NaHCO₃(1.0 mL). The mixture was extracted with EtOAc (2×3 mL). The combined organic layers were dried over anhydrous sodium sulfate, concentrated and purified by prep-HPLC [Phenomenex luna C18 150×25 mm×10 μm; A:water (FA), B: ACN; 26%-56% over 15 min] to afford the title compound (7.03 mg, 33% yield, 0.42 HCOOH) as white solid; ¹H NMR (400 MHz, METHANOL-d4) δ=8.06-7.97 (m, 1H), 7.71-7.61 (m, 1H), 7.31-7.18 (m, 2H), 7.00-6.91 (m, 1H), 4.75-4.67 (m, 2H), 4.65-4.55 (m, 1H), 4.45-4.30 (m, 3H), 4.01-3.86 (m, 1H), 3.63-3.52 (m, 2H), 3.49-3.40 (m, 1H), 2.95-2.75 (m, 2H), 2.63-2.50 (m, 2H), 2.48-2.34 (m, 2H), 2.2-2.13 (m, 3H), 2.08-1.91 (m, 3H), 1.89-1.77 (m, 2H), 1.58-1.42 (m, 1H), 0.87-0.69 (m, 3H); LCMS (ESI, M+1): m/z=667.5.

### Example 119

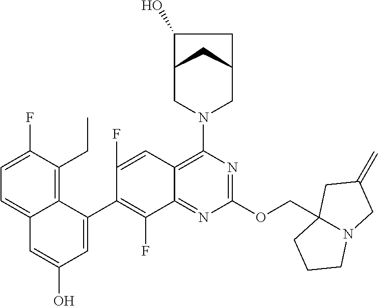 [SMILES: C=C1CN2CCCC2(COc2nc(N3C[C@@H]4C[C@H](C3)[C@H](O)C4)c3cc(F)c(-c4cc(O)cc5ccc(F)c(CC)c45)c(F)c3n2)C1]

### (1R,5R,6R)-3-(7-(8-ethyl-7-fluoro-3-hydroxynaphthalen-1-yl)-6,8-difluoro-2-((2-methylenetetrahydro-1H-pyrrolizin-7a(5H)-yl)methoxy)quinazolin-4-yl)-3-azabicyclo[3.2.1]octan-6-ol

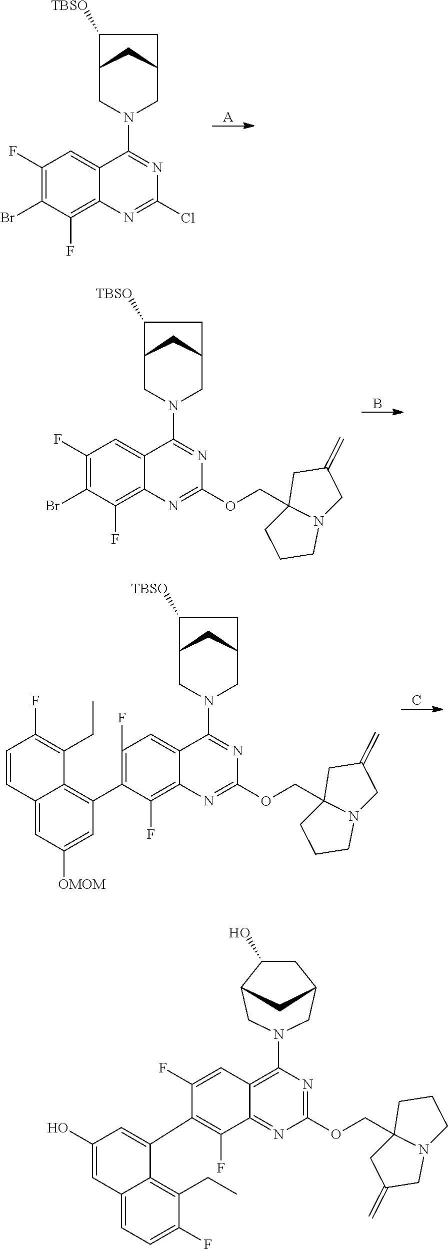

Step A. 7-bromo-4-((1R,5R,6R)-6-((tert-butyldimethylsilyl)oxy)-3-azabicyclo[3.2.1]octan-3-yl)-6,8-difluoro-2-((2-methylenetetrahydro-1H-pyrrolizin-7a(5H)-yl)methoxy)quinazoline: To a mixture of 7-bromo-4-((1R,5R,6R)-6-((tert-butyldimethylsilyl)oxy)-3-azabicyclo[3.2.1]octan-3-yl)-2-chloro-6,8-difluoroquinazoline (214 mg, 1.0 equiv), (2-methylenetetrahydro-1H-pyrrolizin-7a(5H)-yl)methanol (94.8 mg, 1.5 equiv) and Cs₂CO₃ (403 mg, 3.0 equiv) in THE (2.0 mL) and DMF (2.0 mL) were added 4 Å molecular sieve (214 mg) and DABCO (46.3 mg, 1 equiv). The reaction was stirred at 40° C. for 16 hours. The mixture was quenched with saturated ammonium chloride solution (20 mL) and extracted with EtOAc (3×5.0 mL). The combined organic layers were washed with brine (2×15 mL), dried over anhydrous sodium sulfate, concentrated and purified by prep-TLC (petroleum ether/ethyl acetate=0/1) to afford the title compound (80.0 mg, 29% yield) as light yellow oil; LCMS (ESI, M+1, M+3): m/z=635.2, 637.1.

Step B. 4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-6,8-difluoro-2-((2-methylenetetrahydro-1H-pyrrolizin-7a(5H)-yl)methoxy)quinazoline: To a mixture of 7-bromo-4-((1R,5R,6R)-6-((tert-butyldimethylsilyl)oxy)-3-azabicyclo[3.2.1]octan-3-yl)-6,8-difluoro-2-((2-methylenetetrahydro-1H-pyrrolizin-7a(5H)-yl)methoxy)quinazoline (50.0 mg, 1.0 equiv), 2-(8-ethyl-7-fluoro-3-(methoxymethoxy)naphthalen-1-yl)-4,4,5,5-tetramethyl-1,3,2-dioxaborolane (34.0 mg, 1.2 equiv) and Cs₂CO₃ (168 μL, 1.5 M, 2.0 equiv) in dioxane (1.0 mL) and water (0.2 mL) were added XPhos (3.8 mg, 0.10 equiv) and Xphos Pd G4 (6.8 mg, 0.10 equiv). The reaction was degassed and purged with nitrogen 3 times. The reaction was stirred at 80° C. for 1 hour. The mixture was diluted with water (10 mL) and extracted with EtOAc (3×3.0 mL). The combined organic layers were washed with brine (2×10 mL), dried over anhydrous sodium sulfate, concentrated and purified by prep-TLC (dichloromethane/methanol=10/1) to afford the title compound (40.0 mg, 61% yield) as brown oil; LCMS (ESI, M+1): m/z=789.7.

Step C. (1R,5R,6R)-3-(7-(8-ethyl-7-fluoro-3-hydroxynaphthalen-1-yl)-6,8-difluoro-2-((2-methylenetetrahydro-1H-pyrrolizin-7a(5H)-yl)methoxy)quinazolin-4-yl)-3-azabicyclo[3.2.1]octan-6-ol: To a solution of 4-((1R,5R,6R)-6-((tert-butyldimethylsilyl)oxy)-3-azabicyclo[3.2.1]octan-3-yl)-7-(8-ethyl-7-fluoro-3-(methoxymethoxy)naphthalen-1-yl)-6,8-difluoro-2-((2-methylenetetrahydro-1H-pyrrolizin-7a(5H)-yl)methoxy)quinazoline (40.0 mg, 1.0 equiv) in MeOH (0.30 mL) was added HCl.MeOH (342 μL, 4 M, 27 equiv). The reaction was stirred at 25° C. for 0.5 hour. The mixture was concentrated and purified by prep-HPLC [Phenomenex luna C18 150×25 mm×10 um; A: water (0.1% FA), B: ACN; 26%-56% over 10 min] to afford the title compound (9.35 mg, 28% yield, 0.57 HCOOH) as white solid; ¹H NMR (400 MHz, METHANOL-d₄) δ=8.06-7.97 (m, 1H), 7.71-7.64 (m, 1H), 7.29 (d, J=2.0 Hz, 1H), 7.25 (t, J=9.2 Hz, 1H), 6.97 (s, 1H), 5.15 (br s, 2H), 4.74-4.62 (m, 2H), 4.52-4.42 (m, 2H), 4.39-4.32 (m, 1H), 4.12-4.03 (m, 1H), 3.67-3.58 (m, 2H), 3.45 (br d, J=12.8 Hz, 2H), 3.05-2.89 (m, 2H), 2.69-2.50 (m, 2H), 2.49-2.36 (m, 2H), 2.34-2.18 (m, 3H), 2.16-1.94 (m, 3H), 1.91-1.77 (m, 2H), 1.48 (br d, J=14.8 Hz, 1H), 0.84-0.77 (m, 3H); LCMS (ESI, M+1): m/z=631.6.

### Example 120

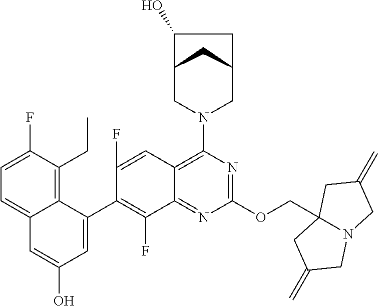 [SMILES: C=C1CN2CC(=C)CC2(COc2nc(N3C[C@@H]4C[C@H](C3)[C@H](O)C4)c3cc(F)c(-c4cc(O)cc5ccc(F)c(CC)c45)c(F)c3n2)C1]

### (1R,5R,6R)-3-(2-((2,6-dimethylenetetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-azabicyclo[3.2.1]octan-6-ol

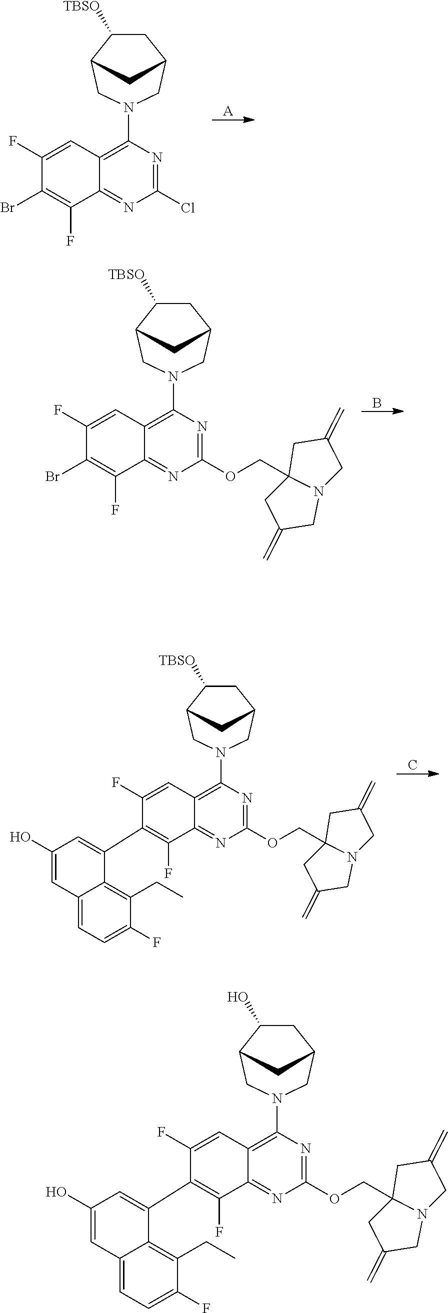

Step A. 7-bromo-4-((1R,5R,6R)-6-((tert-butyldimethylsilyl)oxy)-3-azabicyclo[3.2.1]octan-3-yl)-2-((2,6-dimethylenetetrahydro-1H-pyrrolizin-7a(5H)-yl)methoxy)-6,8-difluoroquinazoline: To a solution of 7-bromo-4-((1R,5R,6R)-6-((tert-butyldimethylsilyl)oxy)-3-azabicyclo[3.2.1]octan-3-yl)-2-chloro-6,8-difluoroquinazoline (100 mg, 1.0 equiv) and (2,6-dimethylenetetrahydro-1H-pyrrolizin-7a(5H)-yl)methanol (47.8 mg, 1.5 equiv) in THE (0.5 mL) and DMF (0.5 mL) were added Cs₂CO₃ (188 mg, 3.0 equiv) and DABCO (21.6 mg, 1.0 equiv). The reaction was stirred at 40° C. for 2 hours. The mixture was diluted with water (5 mL) and extracted with EtOAc (2×10 mL). The combined organic layers were dried over anhydrous sodium sulfate, concentrated and purified by reversed phase flash chromatography [C18, 0.1% formic acid condition] to afford the title compound (82.0 mg, 65% yield) as yellow oil; LCMS (ESI, M+1, M+3): m/z=647.2, 649.2.

Step B. 4-(4-((1R,5R,6R)-6-((tert-butyldimethylsilyl)oxy)-3-azabicyclo[3.2.1]octan-3-yl)-2-((2,6-dimethylenetetrahydro-1H-pyrrolizin-7a(5H)-yl)methoxy)-6,8-difluoroquinazolin-7-yl)-5-ethyl-6-fluoronaphthalen-2-ol: To a mixture of 7-bromo-4-((1R,5R,6R)-6-((tert-butyldimethylsilyl)oxy)-3-azabicyclo[3.2.1]octan-3-yl)-2-((2,6-dimethylenetetrahydro-1H-pyrrolizin-7a(5H)-yl)methoxy)-6,8-difluoroquinazoline (67.0 mg, 1.0 equiv), 5-ethyl-6-fluoro-4-(4,4,5,5-tetramethyl-1,3,2-dioxaborolan-2-yl)naphthalen-2-ol (49.1 mg, 1.5 equiv) and Cs₂CO₃ (1.5 M, 3.0 equiv) in dioxane (1.0 mL) were added RuPhos (14.5 mg, 0.3 equiv) and RuPhos Pd G3 (8.65 mg, 0.1 equiv). The reaction was degassed and purged with nitrogen 3 times. The reaction was stirred at 90° C. for 2 hours. The mixture was diluted with water (5.0 mL) and extracted with EtOAc (2×8.0 mL). The combined organic layers were dried over anhydrous sodium sulfate, concentrated and purified by reversed phase flash chromatography [C18, 0.1% formic acid condition] to afford the title compound (30 mg, 33% yield) as yellow solid; LCMS (ESI, M+1): m/z=757.4.

Step C. (1R,5R,6R)-3-(2-((2,6-dimethylenetetrahydro-1H-pyrrolizin-7a(5H)-yl)methoxy)-7-(8-ethyl-7-fluoro-3-hydroxynaphthalen-1-yl)-6,8-difluoroquinazolin-4-yl)-3-azabicyclo[3.2.1]octan-6-ol: To a solution of 4-(4-((1R,5R,6R)-6-((tert-butyldimethylsilyl)oxy)-3-azabicyclo[3.2.1]octan-3-yl)-2-((2,6-dimethylenetetrahydro-1H-pyrrolizin-7a(5H)-yl)methoxy)-6,8-difluoroquinazolin-7-yl)-5-ethyl-6-fluoronaphthalen-2-ol (25.0 mg, 1.0 equiv) in MeOH (0.25 mL) was added HCl.MeOH (4 M, 30 equiv). The reaction was stirred at 20° C. for 1 hour. The mixture was concentrated. The residue was diluted with MeOH (1.0 mL). NaHCO₃(0.3 g) was added into the mixture. The mixture was stirred at 20° C. for 0.5 hour. The mixture was filtered, concentrated and purified by prep-HPLC [Phenomenex luna C18 150×25 mm×10 μm; A:water (FA); B: ACN; B %: 22%-52% over 10 min] to afford the title compound (4.33 mg, 19% yield, 0.45 HCOOH) as yellow solid; ¹H NMR (400 MHz, DMSO-d₆) δ=8.29 (d, J=2.4 Hz, 1H), 7.80-7.73 (m, 1H), 7.39-7.31 (m, 2H), 7.03-6.98 (m, 1H), 4.98-4.88 (m, 4H), 4.50-4.39 (m, 1H), 4.36-4.27 (m, 1H), 4.24-4.14 (m, 1H), 4.13-3.99 (m, 2H), 3.62 (br s, 2H), 3.45 (br d, J=11.6 Hz, 1H), 3.30-3.23 (m, 1H), 3.22-3.12 (m, 2H), 2.71-2.59 (m, 2H), 2.47-2.39 (m, 3H), 2.38-2.27 (m, 2H), 2.17-2.03 (m, 2H), 1.72-1.61 (m, 2H), 1.39-1.30 (m, 1H), 0.79-0.68 (m, 3H); LCMS (ESI, M+1): m/z=643.3.

### Example 121

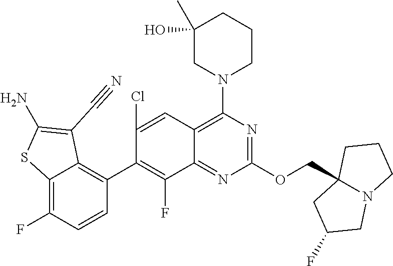 [SMILES: C[C@@]1(O)CCCN(c2nc(OC[C@@]34CCCN3C[C@H](F)C4)nc3c(F)c(-c4ccc(F)c5sc(N)c(C#N)c45)c(Cl)cc23)C1]

### 2-amino-4-(6-chloro-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile

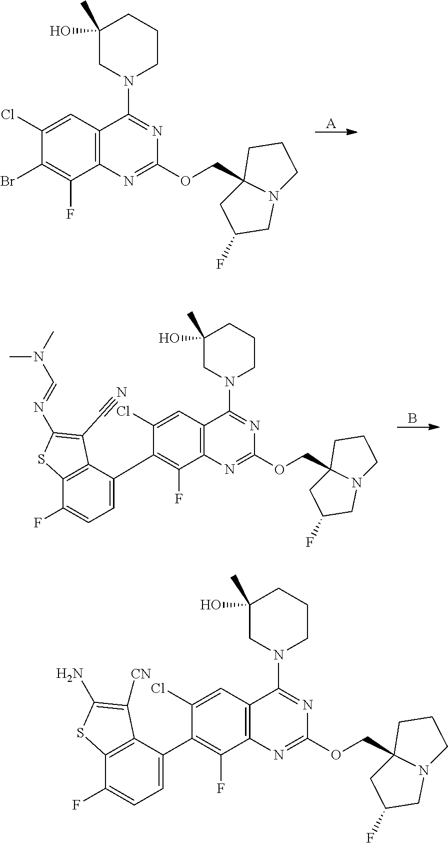

Step A. (E)-N′-(4-(6-chloro-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-3-cyano-7-fluorobenzo[b]thiophen-2-yl)-N,N-dimethylformimidamide: To a mixture of (R)-1-(7-bromo-6-chloro-8-fluoro-2-(((2R,7aS)-2-fluorotetrahydro-1H-pyrrolizin-7a(5H)-yl)methoxy)quinazolin-4-yl)-3-methylpiperidin-3-ol (160 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (202 mg, 1.8 equiv) and aqueous K₃PO₄ (0.8 mL, 1.5 M) in methoxycyclopentane was added Aphos Pd G3 (20.0 mg, 0.10 equiv). The reaction was degassed and purged with nitrogen 3 times. The reaction was stirred at 90° C. for 3 hours. The mixture was quenched with H₂O (5 mL) and extracted with EtOAc (2×5 mL). The combined organic layers were washed with brine (10 mL), dried over anhydrous sodium sulfate, concentrated, and purified by reversed phase flash chromatography [C18, 0.1% formic acid condition] to afford the title compound (20.0 mg, 9.0% yield) as white solid; LCMS (ESI, M+1): m/z=698.2.

Step B. 2-amino-4-(6-chloro-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile: To a mixture of (E)-N′-(4-(6-chloro-8-fluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-3-cyano-7-fluorobenzo[b]thiophen-2-yl)-N,N-dimethylformimidamide (50.0 mg, 1.0 equiv) and DMAc (1 mL) was added aqueous K₃PO₄ (0.20 mL, 1.5 M). The reaction was stirred at 90° C. for 0.5 hours. The mixture was quenched with H₂O (10 mL) and extracted with EtOAc (2×3 mL). The combined organic layers were washed with brine (3 mL), dried over anhydrous sodium sulfate, concentrated and purified with prep-HPLC [column: Waters xbridge 150×25 mm×10 μm; A: water (NH₄HCO₃), B: ACN, B %: 44%-74% over 10 min] to afford the title compound (12.8 mg, 28% yield) as yellow solid; ¹H NMR (400 MHz, METHANOL-d₄) δ=8.05 (d, J=10.4 Hz, 1H), 7.20 (dt, J=5.2, 8.4 Hz, 1H), 7.04 (t, J=9.2 Hz, 1H), 5.41-5.23 (m, 1H), 4.39-4.30 (m, 1H), 4.24 (br dd, J=3.2, 10.4 Hz, 2H), 4.04 (br d, J=13.4 Hz, 1H), 3.53 (br d, J=13.2 Hz, 1H), 3.47-3.33 (m, 2H), 3.29-3.19 (m, 2H), 3.04 (dt, J=5.6, 9.2 Hz, 1H), 2.44-2.08 (m, 4H), 2.06-1.96 (m, 2H), 1.95-1.67 (m, 4H), 1.31-1.22 (m, 3H); LCMS (ESI, M+1): m/z=643.2.

### Example 122A

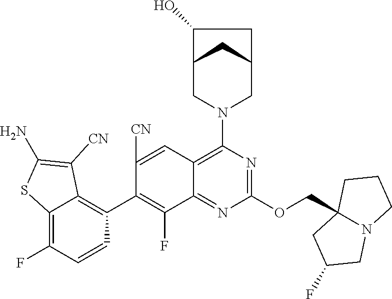

### 2-amino-4-(6-chloro-8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)quinazolin-7-yl)-7-fluorobenzo[b]thiophene-3-carbonitrile

### Example 122B

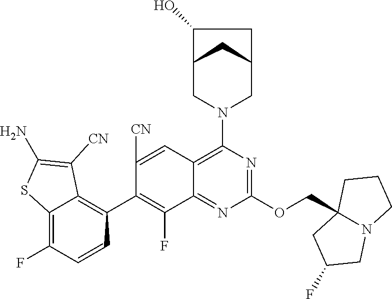

### 2-amino-4-(6-chloro-8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)quinazolin-7-yl)-7-fluorobenzo[b]thiophene-3-carbonitrile

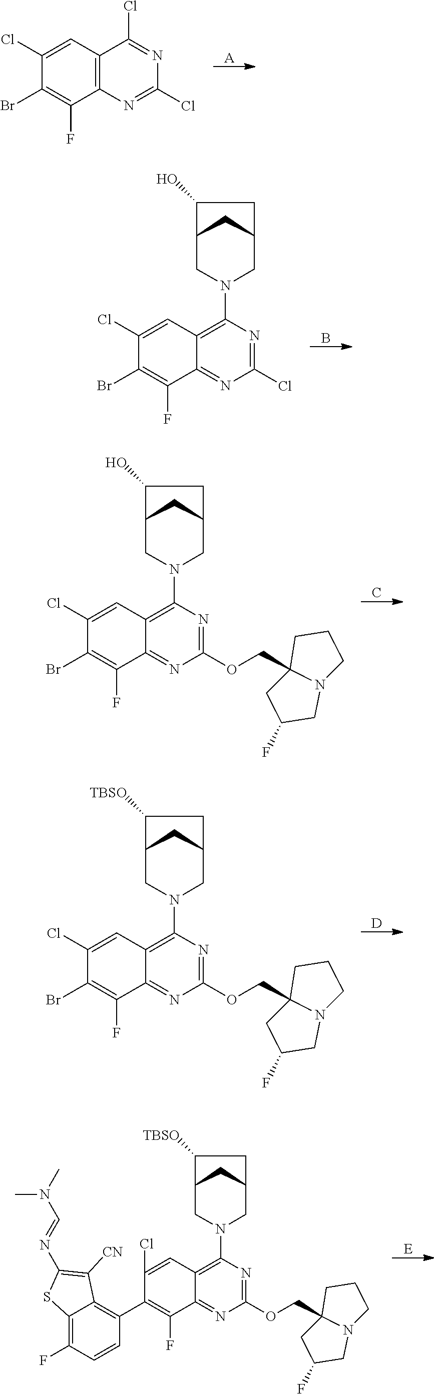

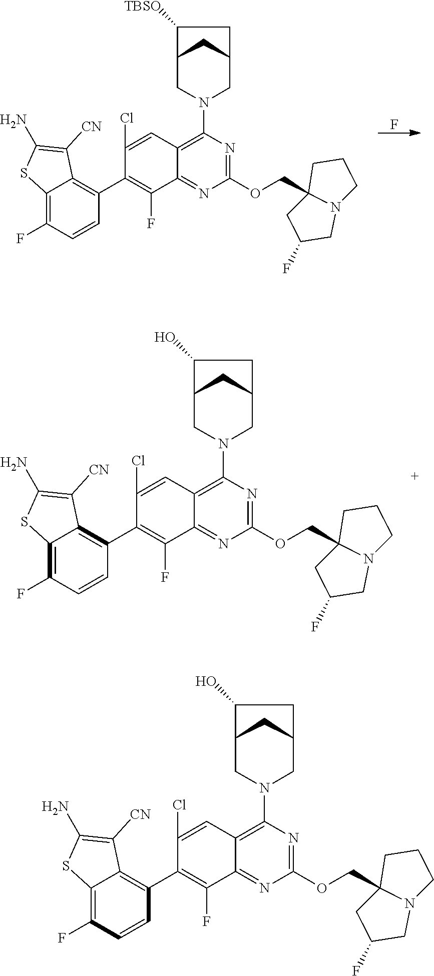

Step A. (1R,5R,6R)-3-(7-bromo-2,6-dichloro-8-fluoroquinazolin-4-yl)-3-azabicyclo[3.2.1]octan-6-ol: To a mixture of 7-bromo-2,4,6-trichloro-8-fluoroquinazoline (2.00 g, 1.0 equiv) and DIEA (2.35 g, 3.0 equiv) in DCM (20 mL) was added (1R,5R,6R)-3-azabicyclo[3.2.1]octan-6-ol (731 mg, 1.0 equiv). The reaction was stirred at −40° C. for 1 hour. The mixture was diluted with water (100 mL) and extracted with EtOAc (2×100 mL). The combined organic layers were dried over anhydrous sodium sulfate and concentrated to afford the title compound (1.90 g, crude) as yellow solid.

Step B. (1R,5R,6R)-3-(7-bromo-6-chloro-8-fluoro-2-(((2R,7aS)-2-fluorotetrahydro-1H-pyrrolizin-7a(5H)-yl)methoxy)guinazolin-4-yl)-3-azabicyclo[3.2.1]octan-6-ol: To a mixture of (1R,5R,6R)-3-(7-bromo-2,6-dichloro-8-fluoroquinazolin-4-yl)-3-azabicyclo[3.2.1]octan-6-ol (700 mg, 1.0 equiv) and ((2R,7aS)-2-fluorotetrahydro-1H-pyrrolizin-7a(5H)-yl)methanol (2.65 g, 10 equiv) was added TEA (505 mg, 3.0 equiv). The reaction was stirred at 90° C. for 16 hours. The mixture was diluted with water (100 mL) and extracted with ethyl acetate (3×50 mL). The combined organic layers were dried over anhydrous sodium sulfate, concentrated, and purified by reversed phase flash chromatography [C18, 0.1% formic acid condition] to afford the title compound (460 mg, 45% yield) as yellow solid. LCMS (ESI, M+1, M+3): m/z=543.1, 545.1.

Step C. 7-bromo-4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazoline: To a solution of (1R,5R,6R)-3-(7-bromo-6-chloro-8-fluoro-2-(((2R,7aS)-2-fluorotetrahydro-1H-pyrrolizin-7a(5H)-yl)methoxy)quinazolin-4-yl)-3-azabicyclo[3.2.1]octan-6-ol (300 mg, 1.0 equiv), TBSCl (166 mg, 2.0 equiv) and imidazole (113 mg, 3.0 equiv) in DMF (6.00 mL) was added DMAP (33.7 mg, 0.5 equiv). The reaction was stirred at 40° C. for 16 hours. The mixture was diluted with water (20 mL) and extracted with ethyl acetate (2×10 mL). The combined organic layers were dried over anhydrous sodium sulfate, concentrated and purified by reversed phase flash chromatography [C18, 0.1% formic acid condition] to afford the title compound (360 mg, 96% yield) as yellow solid; LCMS (ESI, M+1, M+3): m/z=656.9, 658.9.

Step D. (E)-N′-(4-(4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)-N,N-dimethylformimidamide: To a solution of 7-bromo-4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazoline (205 mg, 1.0 equiv) and (E)-N′-(3-cyano-7-fluoro-4-(4,4,5,5-tetramethyl-1,3,2-dioxaborolan-2-yl)benzo[b]thiophen-2-yl)-N,N-dimethylformimidamide (140 mg, 1.2 equiv) in CPME (2 mL) were added aqueous K₃PO₄ (0.4 mL, 1.5 M) and cataCXium-A-Pd-G₃ (22.7 mg, 0.1 equiv). The reaction was degassed and purged with N₂ for 3 times. The reaction was stirred at 90° C. for 3 hours. The mixture was diluted with water (20 mL) and extracted with ethyl acetate (2×10 mL). The combined organic layers were dried over anhydrous sodium sulfate, concentrated, and purified by reversed phase flash chromatography [C18, 0.1% formic acid condition] twice to afford the title compound (200 mg, 56% yeild) as yellow solid; LCMS (ESI, M+1): m/z=824.5.

Step E. 2-amino-4-(4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of (E)-N′-(4-(4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)-N,N-dimethylformimidamide (155 mg, 1.0 equiv) in DMAC (2.00 mL) was added aqueous K₃PO₄ (2.0 mL, 2 M). The reaction was stirred at 80° C. for 1 hour. The mixture was diluted with water (20 mL) and extracted with ethyl acetate (2×10 mL). The combined organic layers were dried over anhydrous sodium sulfate, concentrated, and purified by reversed phase flash chromatography [C18, 0.1% formic acid condition] to afford the title compound (180 mg, 86% yield) as yellow solid.

Step F. 2-amino-4-(6-chloro-8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)quinazolin-7-yl)-7-fluorobenzo[b]thiophene-3-carbonitrile: A solution of 2-amino-4-(4-((1R,5R,6R)-6-((tert-butyldimethylsilyl)oxy)-3-azabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (120 mg, 1.0 equiv) in HCl.MeOH (2.00 mL) was stirred at 10° C. for 1 hour. The mixture was concentrated, basified with saturated NaHCO₃ solution (20 mL), and extracted with ethyl acetate (2×10 mL). The combined organic layers were dried over anhydrous sodium sulfate concentrated, and purified with reversed phase flash chromatography [C18, 0.1% formic acid condition] followed by prep-HPLC [column: Phenomenex luna C18 150×25 mm×10 μm; A: water (FA), B: ACN; B %:18%-48% over 10 min] to afford two peaks of the title compounds.

Example 122A (19.1 mg, 17% yield, 0.38 HCOOH) as white solid; ¹H NMR (400 MHz, METHANOL-d₄) δ=8.23 (d, J=1.2 Hz, 1H), 7.19 (dd, J=5.2, 8.4 Hz, 1H), 7.03 (t, J=8.8 Hz, 1H), 5.49-5.30 (m, 1H), 4.75-4.61 (m, 2H), 4.48-4.41 (m, 1H), 4.37-4.26 (m, 2H), 3.68-3.54 (m, 2H), 3.51-3.39 (m, 3H), 3.16 (dt, J=5.2, 9.6 Hz, 1H), 2.52-2.31 (m, 3H), 2.30-2.15 (m, 3H), 2.15-2.05 (m, 2H), 2.01-1.91 (m, 1H), 1.90-1.76 (m, 2H), 1.49-1.40 (m, 1H); LCMS (ESI, M+1): m/z=655.2; SFC: t_{R}: 2.143 min, 98.2% de, ChiralpakAD-3 50×4.6 mm I.D., 3 μm column A: EtOH (0.05% DEA), B: C02, 3 mL/min, 220 nm.

Example 122B (7.72 mg, 6% yield, 0.26 HCOOH) as pink solid; ¹H NMR (400 MHz, METHANOL-d₄) δ=8.29 (d, J=1.2 Hz, 1H), 7.23 (s, 1H), 7.07-6.99 (m, 1H), 5.47-5.27 (m, 1H), 4.75 (br d, J=11.6 Hz, 1H), 4.51-4.41 (m, 1H), 4.40-4.27 (m, 3H), 3.57-3.33 (m, 5H), 3.17-3.06 (m, 1H), 2.51-2.32 (m, 2H), 2.32-2.15 (m, 4H), 2.14-2.01 (m, 2H), 2.01-1.89 (m, 1H), 1.87-1.77 (m, 2H), 1.42 (br dd, J=2.4, 13.2 Hz, 1H); LCMS (ESI, M+1): m/z=655.2. SFC: t_{R}=1.990 min, 92.0% de, Chiralpak AD-3 50×4.6 mm I.D., 3 μm column A: EtOH (0.05% DEA), B: C02, 3 mL/min, 220 nm.

### Example 123

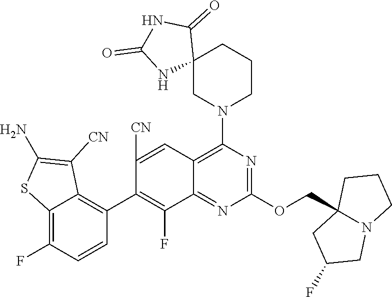 [SMILES: N#Cc1cc2c(N3CCC[C@]4(C3)NC(=O)NC4=O)nc(OC[C@@]34CCCN3C[C@H](F)C4)nc2c(F)c1-c1ccc(F)c2sc(N)c(C#N)c12]

### 2-amino-4-(6-chloro-4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

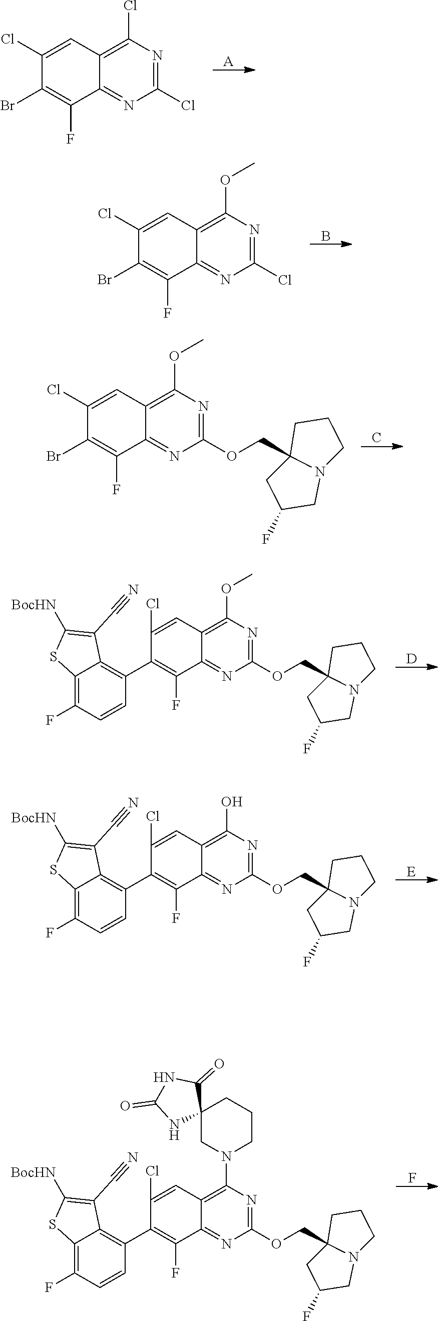

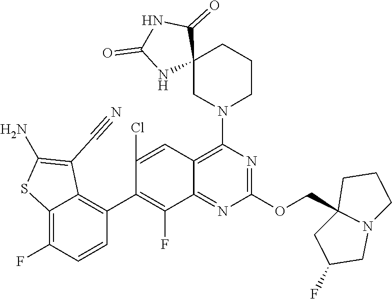 [SMILES: N#Cc1c(N)sc2c(F)ccc(-c3c(Cl)cc4c(N5CCC[C@]6(C5)NC(=O)NC6=O)nc(OC[C@@]56CCCN5C[C@H](F)C6)nc4c3F)c12]

Step A. 7-bromo-2,6-dichloro-8-fluoro-4-methoxyquinazoline: To a solution of 7-bromo-2,4,6-trichloro-8-fluoroquinazoline (60.0 g, 1.0 equiv) in MeOH (600 mL) was added NaOMe (32.7 g, 1.0 equiv, 30% purity) at −40° C. The reaction was stirred at −40° C. for 4 hours. The mixture was quenched with water (3.0 L) and extracted with EtOAc (3×2.0 L). The combined organic layers were washed with brine (3.0 L), dried over anhydrous sodium sulfate and concentrated to afford the title compound (53.0 g, 89% yield) as brown solid; ¹H NMR (400 MHz, DMSO-d₆) δ=8.19 (d, J=2.0 Hz, 1H), 4.18 (s, 3H).

Step B. 7-bromo-6-chloro-8-fluoro-2-(((2R,7aS)-2-fluorotetrahydro-1H-pyrrolizin-7a(5H)-yl)methoxy)-4-methoxyquinazoline: To a mixture of 7-bromo-2,6-dichloro-8-fluoro-4-methoxyquinazoline (53.0 g, 1.0 equiv), DABCO (9.12 g, 0.5 equiv) and Cs₂CO₃ (159 g, 3.0 equiv) in DMF (500 mL) and THE (500 mL) was added ((2R,7aS)-2-fluorotetrahydro-1H-pyrrolizin-7a(5H)-yl)methanol (31.1 g, 1.2 equiv). The reaction was stirred at 25° C. for 4 hours. The mixture was quenched with water (2.50 L) at 25° C. and filtered. The filter cake was triturated with EtOAc (100 mL) at 25° C. to afford the title compound (30 g, 31% yield) as white solid; LCMS (ESI, M+1, M+3): m/z=448.2, 450.2.

Step C. tert-butyl (4-(6-chloro-8-fluoro-2-(((2R,7aS)-2-fluorotetrahydro-1H-pyrrolizin-7a(5H)-yl)methoxy)-4-methoxyquinazolin-7-yl)-3-cyano-7-fluorobenzo[b]thiophen-2-yl)carbamate: To a solution of 7-bromo-6-chloro-8-fluoro-2-(((2R,7aS)-2-fluorotetrahydro-1H-pyrrolizin-7a(5H)-yl)methoxy)-4-methoxyquinazoline (10.0 g, 1.0 equiv) and tert-butyl (3-cyano-4-(5,5-dimethyl-1,3,2-dioxaborinan-2-yl)-7-fluorobenzo[b]thiophen-2-yl)carbamate (22.5 g, 2.5 equiv) in toluene (500 mL) were added Pd(DPEphos)Cl₂ (2.30 g, 0.15 equiv) and Cs₂CO₃ (21.8 g, 3.0 equiv). The reaction was degassed and purged with nitrogen for 3 times. The reaction was stirred at 110° C. for 3 hours under nitrogen atmosphere. The mixture was quenched with water (500 mL) at 25° C. and extracted with EtOAc (2×250 mL). The combined organic layers were washed with brine (200 mL), dried over anhydrous sodium sulfate, concentrated and purified with prep-HPLC [Phenomenex luna C18 75×30 mm×3 μm; A: water (FA), B: ACN, B %: 40%-70% over 13 min] to afford the title compound (2.70 g, 17% yield) as yellow solid; LCMS (ESI, M+1): m/z=660.3.

Step D. tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-methoxyquinazolin-7-yl)-3-cyano-7-fluorobenzo[b]thiophen-2-yl)carbamate in DMAC (30 mL) was added NaSEt (1.72 g, 5.0 equiv). The reaction was stirred at 60° C. for 1 hour. The mixture was quenched with water (80 mL) at 0° C. and extracted with DCM (3×50 mL). The combined organic layers were washed with brine (50 mL), dried over Na₂SO₄ and concentrated to afford the title compound (1.80 g, 46% yield) as yellow solid; LCMS (ESI, M+1): m/z=646.2.

Step E. tert-butyl (4-(6-chloro-4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)guinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (80 mg, 1.0 equiv) in DMSO (0.8 mL) were added TEA (37.6 mg, 3.0 equiv) and PyBOP (96.7 mg, 1.5 equiv). The reaction was stirred at 25° C. for 0.5 hour. Then (5R)-1,3,9-triazaspiro[4.5]decane-2,4-dione (41.9 mg, 2.0 equiv) was added to the mixture. The reaction was stirred at 25° C. for 9.5 hours. The reaction mixture was diluted with water (10 mL) and extracted with EtOAc (2×10 mL). The combined organic layers were washed with brine (2×10 mL), dried over anhydrous sodium sulfate, concentrated and purified by reversed phase flash chromatography [C18, 0.1% formic acid condition] to afford the title compound (40.0 mg, 39% yield) as yellow solid; LCMS (ESI, M+1): m/z=797.2.

Step F. 2-amino-4-(6-chloro-4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)guinazolin-7-yl)-7-fluorobenzo[b]thiophene-3-carbonitrile: A solution of tert-butyl (4-(6-chloro-4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30 mg, 1.0 equiv) in HCl.MeOH (0.5 mL) was stirred at 25° C. for 8 hours. The mixture was purified by Prep-HPLC [Phenomenex luna C18 150×25 mm×10 μm; A: water(FA), B: ACN; B %: 22%-52% B over 9 min] to afford the title compound (3.73 mg, 14% yield, 0.18 HCOOH) as off-white solid; ¹H NMR (400 MHz, METHANOL-d₄) δ=7.88 (br s, 1H), 7.16-7.06 (m, 1H), 6.94 (t, J=8.8 Hz, 1H), 5.39-5.18 (m, 1H), 4.38-4.17 (m, 4H), 3.65-3.26 (m, 5H), 3.11-2.97 (m, 1H), 2.43-1.72 (m, 10H); LCMS (ESI, M+1): m/z=697.2.

### Example 124A

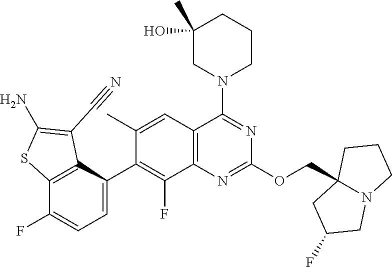

### 2-amino-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)-6-methylquinazolin-7-yl)benzo[b]thiophene-3-carbonitrile

### Example 124B

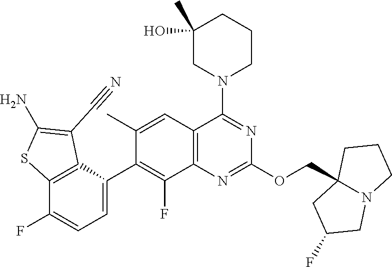

### 2-amino-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)-6-methylquinazolin-7-yl)benzo[b]thiophene-3-carbonitrile

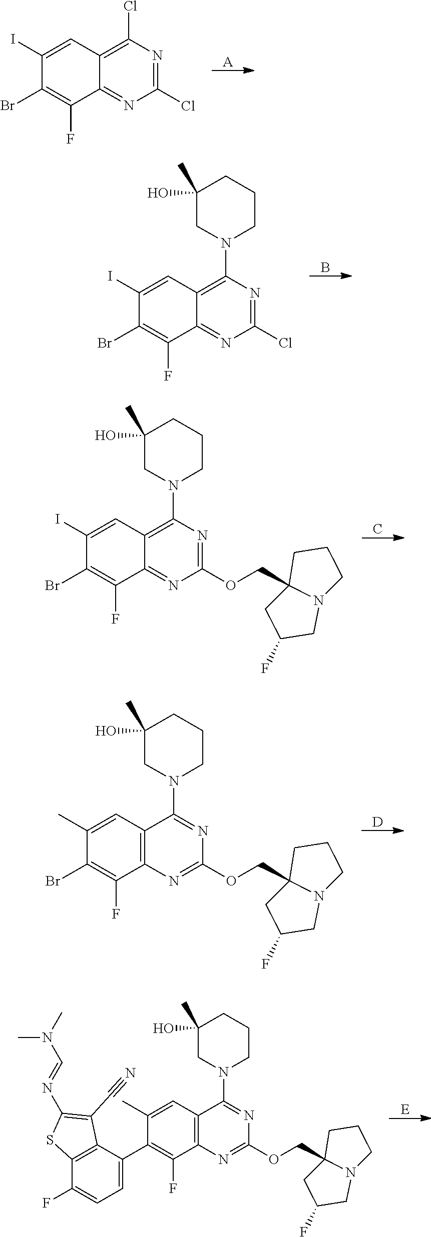

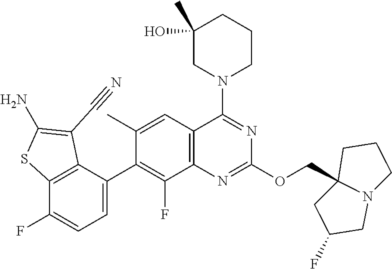 [SMILES: Cc1cc2c(N3CCC[C@@](C)(O)C3)nc(OC[C@@]34CCCN3C[C@H](F)C4)nc2c(F)c1-c1ccc(F)c2sc(N)c(C#N)c12]

Step A. (R)-1-(7-bromo-2-chloro-8-fluoro-6-iodoquinazolin-4-yl)-3-methylpiperidin-3-ol: To a solution of 7-bromo-2,4-dichloro-8-fluoro-6-iodoquinazoline (1.10 g, 1.0 equiv) and DIEA (1.0 g, 3.0 equiv) in DCM (10 mL) was added (R)-3-methylpiperidin-3-ol hydrochloride (0.40 g, 1.0 equiv). The reaction was stirred at −40° C. for 2 hours. The mixture was diluted with water (30 mL) and extracted with DCM (3×30 mL). The combined organic layers were dried over anhydrous sodium sulfate, concentrated, and purified with column chromatography (SiO₂, Petroleum ether/Ethyl acetate=20/1 to 3/1) to afford the title compound (1.14 g, 85% yield) as yellow solid; LCMS (ESI, M+1, M+3): m/z=499.9, 501.9.

Step B. (R)-1-(7-bromo-8-fluoro-2-(((2R,7aS)-2-fluorotetrahydro-1H-pyrrolizin-7a(5H)-yl)methoxy)-6-iodoquinazolin-4-yl)-3-methylpiperidin-3-ol: A mixture of (R)-1-(7-bromo-2-chloro-8-fluoro-6-iodoquinazolin-4-yl)-3-methylpiperidin-3-ol (1.00 g, 1.0 equiv) and ((2R,7aS)-2-fluorotetrahydro-1H-pyrrolizin-7a(5H)-yl)methanol (3.18 g, 10.0 equiv) was stirred at 100° C. for 24 hours. The mixture was triturated with water at 20° C. for 2 hrs. The residue was purified with prep-HPLC [Phenomenex luna C18 150×40 mm×15 μm; A: water (0.1% FA), B: ACN, B %: 0%-61% over 15 min] to afford the title compound (282 mg, 18% yield) as brown solid; LCMS (ESI, M+1, M+3): m/z=623.0, 625.0.

Step C. (R)-1-(7-bromo-8-fluoro-2-(((2R,7aS)-2-fluorotetrahydro-1H-pyrrolizin-7a(5H)-yl)methoxy)-6-methylquinazolin-4-yl)-3-methylpiperidin-3-ol: To a mixture of (R)-1-(7-bromo-8-fluoro-2-(((2R,7aS)-2-fluorotetrahydro-1H-pyrrolizin-7a(5H)-yl)methoxy)-6-iodoquinazolin-4-yl)-3-methylpiperidin-3-ol (282 mg, 1.0 equiv), 2,4,6-trimethyl-1,3,5,2,4,6-trioxatriborinane (50%, 190 μL, 1.5 equiv) and aqueous K₃PO₄ (0.1 mL, 1.5 M, 3.0 equiv) in dioxane (3 mL) was added Pd(dppf)Cl₂ (33.1 mg, 0.1 equiv). The reaction was degassed and purged with nitrogen 3 times. The reaction was stirred at 60° C. for 24 hours. The mixture was quenched by H₂O (30 mL) and extracted with EtOAc (3×10 mL). The combined organic layers were washed with brine (15 mL), dried over anhydrous sodium sulfate, concentrated and purified with prep-HPLC [SW80 spherical C18 20-45 μm, 100 Å; A: water (FA), B: ACN; B %: 0%-100% over 30 min] to afford the title compound (119 mg, 45% yield) as brown solid; LCMS (ESI, M+1, M+3): m/z=511.2, 513.2.

Step D. (E)-N′-(3-cyano-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)-6-methylquinazolin-7-yl)benzo[b]thiophen-2-yl)-N,N-dimethylformimidamide: To a solution of (R)-1-(7-bromo-8-fluoro-2-(((2R,7aS)-2-fluorotetrahydro-1H-pyrrolizin-7a(5H)-yl)methoxy)-6-methylquinazolin-4-yl)-3-methylpiperidin-3-ol (80 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (117 mg, 2.0 equiv.) and aqueous K₃PO₄ (0.3 mL, 1.5 M, 3.0 equiv) in THE (0.5 mL) was added Aphos-Pd-G₃ (10 mg, 0.10 equiv). The reaction was stirred at 60° C. for 3 hours under nitrogen atmosphere. The mixture was quenched by H₂O (20 mL) and extracted with DCM (3×20 mL). The combined organic layers were dried over anhydrous sodium sulfate, concentrated and purified by prep-HPLC [SW80 spherical C18 20-45 m, 100 Å; A: water (FA), B: ACN; B %: 0%-100% over 30 min) to afford the title compound (83.0 mg, 73% yield) as yellow solid; LCMS (ESI, M+1): m/z=678.3.

Step E. 2-amino-7-fluoro-4-((S)-8-fluoro-2-(((2R,7aS)-2-fluorotetrahydro-1H-pyrrolizin-7a(5H)-yl)methoxy)-4-((R)-3-hydroxy-3-methylpiperidin-1-yl)-6-methylquinazolin-7-yl)benzo[b]thiophene-3-carbonitrile: To a mixture of (E)-N′-(3-cyano-7-fluoro-4-(8-fluoro-2-(((2R,7aS)-2-fluorotetrahydro-1H-pyrrolizin-7a(5H)-yl)methoxy)-4-((R)-3-hydroxy-3-methylpiperidin-1-yl)-6-methylquinazolin-7-yl)benzo[b]thiophen-2-yl)-N,N-dimethylformimidamide (50.0 mg, 1.0 equiv) and DMAc (5.0 mL) was added aqueous K₃PO₄ (5.02 mL, 1.5 M, 102 equiv). The reaction was stirred at 80° C. for 3 hours. The mixture was quenched by H₂O (5 mL) and extracted with EtOAc (3×50 mL). The combined organic layers were washed with brine (50 mL), dried over anhydrous sodium sulfate, concentrated and purified by prep-HPLC [Phenomenex luna C18 150×25 mm×10 μm; A: water (FA), B: ACN; B %: 24%-48% over 8 min], SFC [DAICEL CHIRALPAK IC(250 mm×30 mm, 5 m); A: CO₂-ACN, B: i-PrOH (0.1% NH₃H₂O); B %: 40%, isocratic elution mode], and prep-HPLC [Phenomenex luna C18 150×25 mm×10 μm; A: water(FA), B: ACN; B %: 18%-48% over 10 min] to afford two peaks of the title compounds.

WX-95970A (6.60 mg, 33% yield), off-white solid; ¹H NMR (400 MHz, METHANOL-d₄) δ=7.78 (s, 1H), 7.14 (dd, J=5.2, 8.4 Hz, 1H), 7.08-6.99 (m, 1H), 5.43-5.26 (m, 1H), 4.39 (d, J=11.2 Hz, 1H), 4.27 (d, J=11.2 Hz, 1H), 4.17 (br d, J=13.2 Hz, 1H), 4.03 (br d, J=13.2 Hz, 1H), 3.56 (d, J=13.2 Hz, 1H), 3.51-3.41 (m, 2H), 3.39-3.34 (m, 2H), 3.15-3.04 (m, 1H), 2.45-2.32 (m, 1H), 2.32-2.25 (m, 1H), 2.22 (br d, J=8.8 Hz, 1H), 2.16 (s, 3H), 2.14-1.98 (m, 3H), 1.97-1.87 (m, 1H), 1.86-1.69 (m, 3H), 1.25 (s, 3H); LCMS (ESI, M+1): m/z=623.3; SFC: t_{R}: 0.761 min, 95% de, column: Chiralpak IC-3 50×4.6 mm I.D., 3 m Mobile phase: Phase A for CO₂, and Phase B for EtOH (0.05% DEA); Gradient elution: 40% EtOH (0.05% DEA) in CO₂ Flow rate: 3 mL/min; Detector: PDA; Column Temp: 35 C; Back Pressure: 100 Bar.

WX-95970B (5.03 mg, 25% yield), off-white solid; ¹H NMR (400 MHz, METHANOL-d₄) δ=7.77 (s, 1H), 7.17 (dd, J=5.2, 8.4 Hz, 1H), 7.04 (dd, J=8.4, 9.6 Hz, 1H), 5.45-5.27 (m, 1H), 4.43-4.36 (m, 1H), 4.35-4.28 (m, 1H), 4.20 (br d, J=13.2 Hz, 1H), 4.05 (br d, J=13.2 Hz, 1H), 3.55 (d, J=13.2 Hz, 1H), 3.51-3.35 (m, 4H), 3.12 (dt, J=5.6, 10.0 Hz, 1H), 2.48-2.34 (m, 1H), 2.25 (br s, 1H), 2.23-2.18 (m, 1H), 2.16 (s, 3H), 2.14-2.02 (m, 3H), 2.00-1.89 (m, 1H), 1.87-1.68 (m, 3H), 1.26 (s, 3H); LCMS (ESI, M+1): m/z=623.3; SFC: t_{R}: 1.196 min, 98% de, column: Chiralpak IC-3 50×4.6 mm I.D., 3 m; Mobile phase: Phase A for CO₂, and Phase B for EtOH (0.05% DEA); Gradient elution: 40% EtOH (0.05% DEA) in CO₂; Flow rate: 3 mL/min; Detector: PDA; Column Temp: 35C; Back Pressure: 100 Bar.

### Example 125

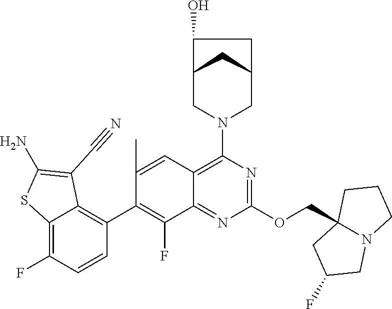 [SMILES: Cc1cc2c(N3C[C@@H]4C[C@H](C3)[C@H](O)C4)nc(OC[C@@]34CCCN3C[C@H](F)C4)nc2c(F)c1-c1ccc(F)c2sc(N)c(C#N)c12]

### 2-amino-7-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)-6-methylquinazolin-7-yl)benzo[b]thiophene-3-carbonitrile

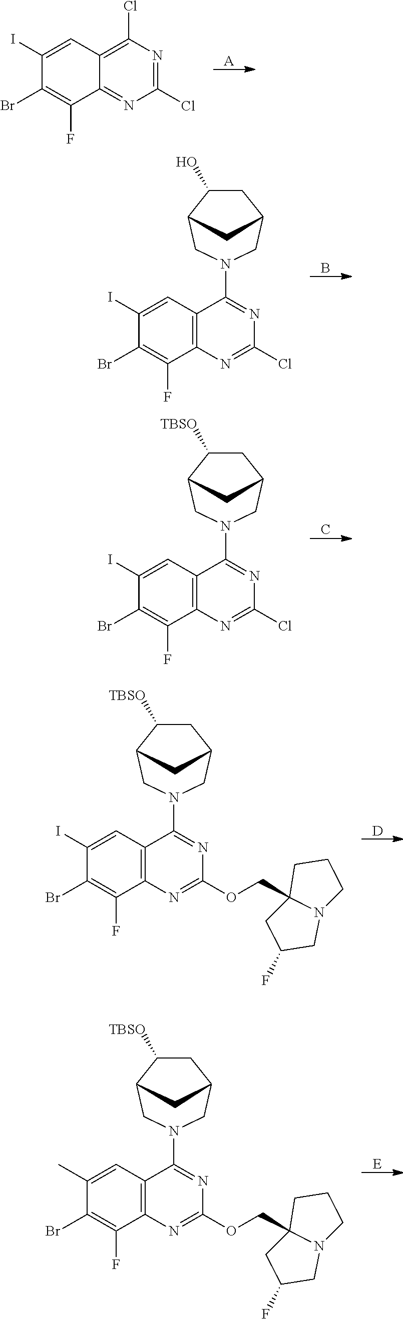

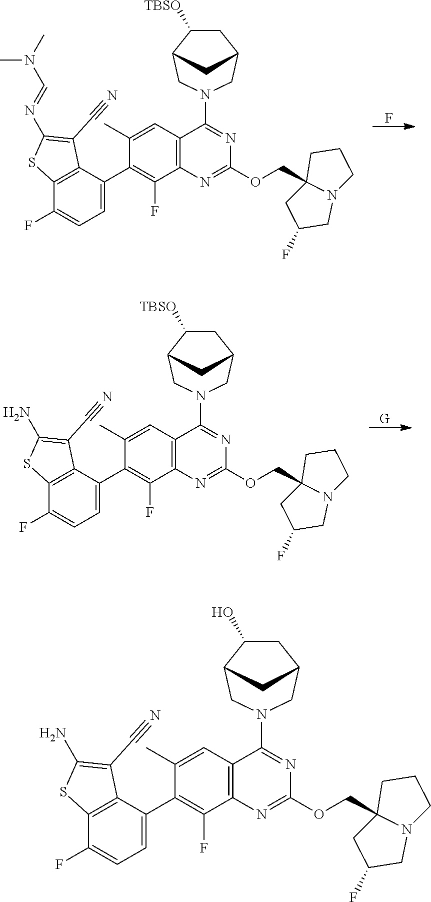

Step A. (1R,5R,6R)-3-(7-bromo-2-chloro-8-fluoro-6-iodoquinazolin-4-yl)-3-azabicyclo[3.2.1]octan-6-ol: To a solution of 7-bromo-2,4-dichloro-8-fluoro-6-iodoquinazoline (1.0 g, 1.0 equiv) and DIEA (1.53 g, 5.0 equiv) in DCM (10 mL) was added (1R,5R,6R)-3-azabicyclo[3.2.1]octan-6-ol (271 mg, 0.90 equiv) at −40° C. The reaction was stirred at −40° C. for 3 hours. The mixture was diluted with water (40 mL) and extracted with DCM (3×30 mL). The combined organic layers were dried over anhydrous sodium sulfate, concentrated and purified with reversed phase flash chromatography [C18, 0.1% formic acid condition] to afford the title compound (1.0 g, 80% yield) as brown solid; LCMS (ESI, M+1, M+3): m/z=511.9, 513.9.

Step B. 7-bromo-4-((1R,5R,6R)-6-((tert-butyldimethylsilyl)oxy)-3-azabicyclo[3.2.1]octan-3-yl)-2-chloro-8-fluoro-6-iodoquinazoline: To a solution of (1R,5R,6R)-3-(7-bromo-2-chloro-8-fluoro-6-iodoquinazolin-4-yl)-3-azabicyclo[3.2.1]octan-6-ol (1.0 g, 1.0 equiv), imidazole (398 mg, 3.0 equiv) and DMAP (119 mg, 0.50 equiv) in DMF (10 mL) was added TBSCl (588 mg, 2.0 equiv). The reaction was stirred at 0-20° C. for 12 hours. The mixture was quenched by H₂O (50 mL) at 0° C. and extracted with EtOAc (3×30 mL). The combined organic layers were dried over anhydrous sodium sulfate, concentrated and purified with column chromatography [SiO₂, Petroleum ether/Ethyl acetate=100/1 to 10/1] to afford the title compound (1.0 g, 76% yield) as yellow solid; LCMS (ESI, M+1, M+3): m/z=626.0, 628.0

Step C. 7-bromo-4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-iodoquinazoline: A mixture of 7-bromo-4-((1R,5R,6R)-6-((tert-butyldimethylsilyl)oxy)-3-azabicyclo[3.2.1]octan-3-yl)-2-chloro-8-fluoro-6-iodoquinazoline (800 mg, 1.0 equiv) and ((2R,7aS)-2-fluorotetrahydro-1H-pyrrolizin-7a(5H)-yl)methanol (4.06 g, 20 equiv) was stirred at 100° C. for 12 hours. The reaction was diluted with water (30 mL) and extracted with ethyl acetate (3×20 mL). The combined organic layers were washed with brine (40 mL), dried over anhydrous sodium sulfate, concentrated, and purified with reversed phase flash chromatography [C18, 0.1% formic acid condition] to afford the title compound (800 mg, 80% yield) as yellow solid; LCMS (ESI, M+1, M+3): m/z=749.2, 751.2.

Step D. 7-bromo-4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazoline: To a mixture of 7-bromo-4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-iodoquinazoline (350 mg, 1.0 equiv), 2,4,6-trimethyl-1,3,5,2,4,6-trioxatriborinane (87.9 mg, 1.5 equiv) and K₂CO₃ (1.5 M, 934 μL, 3.0 equiv) in dioxane (4.0 mL) was added Pd(dppf)Cl₂ (34.2 mg, 0.1 equiv). The reaction was degassed and purged with nitrogen 3 times. The reaction was stirred at 60° C. for 12 hours. The mixture was diluted with water (5.0 mL) and extracted with EtOAc (3×5.0 mL). The combined organic layers were washed with brine (15 mL), dried over anhydrous sodium sulfate, concentrated, and purified with reversed phase flash chromatography [C18, 0.1% formic acid condition] to afford the title compound (130 mg, 43% yield) as yellow solid; LCMS (ESI, M+1, M+3): m/z=637.3, 639.3

Step E. (E)-N′-(4-(4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)-N,N-dimethylformimidamide: To a mixture of 7-bromo-4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazoline (200 mg, 1.0 equiv), (E)-N-(3-cyano-7-fluoro-4-(4,4,5,5-tetramethyl-1,3,2-dioxaborolan-2-yl)benzo[b]thiophen-2-yl)-N,N-dimethylformimidamide (351 mg, 3.0 equiv) and Cs₂CO₃ (1.5 M, 627 μL, 3 equiv) in methoxycyclopentane (3.0 mL) was added CataCXium A Pd G3 (22.8 mg, 0.10 equiv). The reaction was degassed and purged with nitrogen 3 times. The reaction was stirred at 100° C. for 2 hours. The mixture was diluted with water (5.0 mL) and extracted with EtOAc (3×5.0 mL). The combined organic layers were washed with brine (15 mL), dried over anhydrous sodium sulfate, concentrated, and purified with prep-TLC [SiO₂, Dichloromethane: Methanol=10/1] to afford the title compound (120 mg, 47% yield) as yellow solid; LCMS (ESI, M+1): m/z=804.5.

Step F. 2-amino-4-(4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile: To a solution of (E)-N′-(4-(4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)-N,N-dimethylformimidamide (120 mg, 1.0 equiv) in DMAC (0.5 mL) was added aqueous K₃PO₄ (0.5 mL 1.5 M). The reaction was stirred at 80° C. for 1 hour. The mixture was diluted with water (3.0 mL) and extracted with EtOAc (3×3.0 mL). The combined organic layers were washed with brine (10 mL), dried over anhydrous sodium sulfate, and concentrated to afford the title compound (100 mg, crude) as yellow solid; LCMS (ESI, M+1): m/z=749.5.

Step G. 2-amino-7-fluoro-4-(8-fluoro-2-(((2R,7aS)-2-fluorotetrahydro-1H-pyrrolizin-7a(5H)-yl)methoxy)-4-((1R,5R,6R)-6-hydroxy-3-azabicyclo[3.2.1]octan-3-yl)-6-methylquinazolin-7-yl)benzo[b]thiophene-3-carbonitrile: To a solution of 2-amino-4-(4-((1R,5R,6R)-6-((tert-butyldimethylsilyl)oxy)-3-azabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile (50.0 mg, 1.0 equiv) in MeOH (0.5 mL) was added HCl.MeOH (4 M, 0.5 mL). The reaction was stirred at 20° C. for 1 hour. The mixture was concentrated under vacuum. The residue was diluted with saturated NaHCO₃ aqueous (5 mL) at 0° C. The mixture was extracted with EtOAc (3×3.0 mL). The combined organic layers were washed with brine (10 mL), dried over anhydrous sodium sulfate, concentrated and purified with prep-HPLC [column: Phenomenex luna C18 150×25 mm×10 μm; A: water (FA); B: ACN; B %: 20%-40% over 10 min] and lyophilized to afford the title compound (25.7 mg, 61% yield, 0.59 HCOOH) as off-white solid; ¹H NMR (400 MHz, METHANOL-d₄) δ=7.93 (s, 1H), 7.16-7.13 (m, 1H), 7.08-6.99 (m, 1H), 5.56-5.32 (m, 1H), 4.79-4.67 (m, 2H), 4.59-4.54 (m, 1H), 4.41-4.36 (m, 1H), 4.35-4.28 (m, 1H), 3.74 (br s, 4H), 3.51-3.42 (m, 1H), 3.28-3.22 (m, 1H), 2.58-2.39 (m, 2H), 2.39-2.29 (m, 2H), 2.15 (s, 7H), 2.06-1.92 (m, 1H), 1.92-1.77 (m, 2H), 1.51-1.37 (m, 1H); LCMS (ESI, M+1): m/z=635.3.

### Example 126

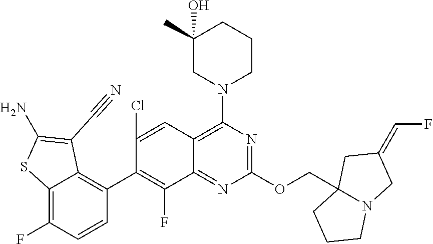 [SMILES: C[C@@]1(O)CCCN(c2nc(OCC34CCCN3C/C(=C\F)C4)nc3c(F)c(-c4ccc(F)c5sc(N)c(C#N)c45)c(Cl)cc23)C1]

### 2-amino-4-(6-chloro-8-fluoro-2-(((Z)-2-(fluoromethylene)tetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile

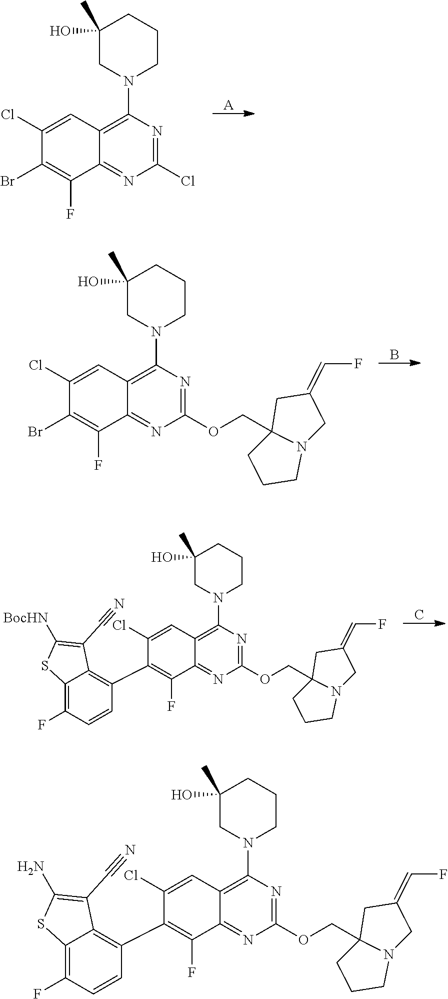

Step A. (3R)-1-(7-bromo-6-chloro-8-fluoro-2-(((Z)-2-(fluoromethylene)tetrahydro-1H-pyrrolizin-7a(5H)-yl)methoxy)guinazolin-4-yl)-3-methylpiperidin-3-ol: To a solution of (R)-1-(7-bromo-2,6-dichloro-8-fluoroquinazolin-4-yl)-3-methylpiperidin-3-ol (250 mg, 1.0 equiv) and (Z)-(2-(fluoromethylene)tetrahydro-1H-pyrrolizin-7a(5H)-yl)methanol (157 mg, 1.5 equiv) in DMF (2.5 mL) and THE (2.5 mL) were added Cs₂CO₃ (597 mg, 3.0 equiv) and DABCO (68.5 mg, 1.0 equiv). The reaction was stirred at 25° C. for 1 hour. The mixture was diluted with water (20 mL) and extracted with EtOAc (3×50 mL). The combined organic layers were washed with brine (30 mL), dried over anhydrous sodium sulfate, concentrated, and purified by reversed phase flash chromatography [C18, 0.1% formic acid condition] to afford the title compound (153 mg, 45% yield) as yellow oil; LCMS (ESI, M+1, M+3): m/z=543.1, 545.1.

Step B. tert-butyl (4-(6-chloro-8-fluoro-2-(((Z)-2-(fluoromethylene)tetrahydro-1H-pyrrolizin-7a(5H)-yl)methoxy)-4-((R)-3-hydroxy-3-methylpiperidin-1-yl)quinazolin-7-yl)-3-cyano-7-fluorobenzo[b]thiophen-2-yl)carbamate: To a mixture of (3R)-1-(7-bromo-6-chloro-8-fluoro-2-(((Z)-2-(fluoromethylene)tetrahydro-1H-pyrrolizin-7a(5H)-yl)methoxy)quinazolin-4-yl)-3-methylpiperidin-3-ol (153 mg, 1.0 equiv), tert-butyl (3-cyano-4-(5,5-dimethyl-1,3,2-dioxaborinan-2-yl)-7-fluorobenzo[b]thiophen-2-yl)carbamate (200 mg, 1.8 equiv) and Cs₂CO₃ (275 mg, 3.0 equiv) in toluene (2 mL) was added Pd(DPEphos)Cl₂ (38.7 mg, 0.2 equiv). The reaction was degassed and purged with nitrogen 3 times. The reaction was stirred at 110° C. for 1 hour. The mixture was quenched water (20 mL) and extracted with DCM (2×30 mL). The combined organic layers were washed with brine (30 mL), dried over anhydrous sodium sulfate, and concentrated and purified by reversed phase flash chromatography [C18, 0.1% formic acid condition] to afford the title compound (100 mg, 46% yield) as yellow oil; LCMS (ESI, M+1): m/z=755.2.

Step C. 2-amino-4-(6-chloro-8-fluoro-2-(((Z)-2-(fluoromethylene)tetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile: To a solution of tert-butyl (4-(6-chloro-8-fluoro-2-(((Z)-2-(fluoromethylene)tetrahydro-1H-pyrrolizin-7a(5H)-yl)methoxy)-4-((R)-3-hydroxy-3-methylpiperidin-1-yl)quinazolin-7-yl)-3-cyano-7-fluorobenzo[b]thiophen-2-yl)carbamate (100 mg, 1.0 equiv) in DCM (2.5 mL) was added TFA (3.84 g, 254 equiv). The reaction was stirred at 25° C. for 1 hour. The mixture was filtered, concentrated and purified by Prep-HPLC [column: Phenomenex luna C18 150×25 mm×10 μm; A: water (0.1% FA); B: ACN, B %: 25%-55% over 9 min] to afford the title compound (41.1 mg, 46% yield) as white solid; ¹H NMR (400 MHz, METHANOL-d₄) δ=8.12 (br s, 1H), 7.28-7.14 (m, 1H), 7.10-6.98 (m, 1H), 6.97-6.67 (m, 1H), 4.75-4.51 (m, 2H), 4.46-4.26 (m, 2H), 4.17-3.98 (m, 2H), 3.79-3.64 (m, 1H), 3.55 (br dd, J=8.0, 13.2 Hz, 1H), 3.46-3.34 (m, 1H), 3.27-3.16 (m, 1H), 2.98 (br d, J=15.6 Hz, 1H), 2.74 (br d, J=15.6 Hz, 1H), 2.46-2.28 (m, 1H), 2.27-2.03 (m, 4H), 1.90-1.67 (m, 3H), 1.38-1.18 (m, 3H); LCMS (ESI, M+1): m/z=655.3.

### Example 127

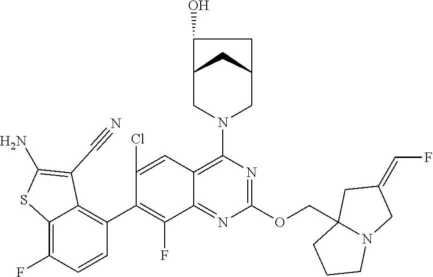 [SMILES: N#Cc1c(N)sc2c(F)ccc(-c3c(Cl)cc4c(N5C[C@@H]6C[C@H](C5)[C@H](O)C6)nc(OCC56CCCN5C/C(=C\F)C6)nc4c3F)c12]

### 2-amino-4-(6-chloro-8-fluoro-2-(((Z)-2-(fluoromethylene)tetrahydro-1H-pyrrolizin-7a(5H)-yl)methoxy)-4-((1R,5R,6R)-6-hydroxy-3-azabicyclo[3.2.1]octan-3-yl)quinazolin-7-yl)-7-fluorobenzo[b]thiophene-3-carbonitrile

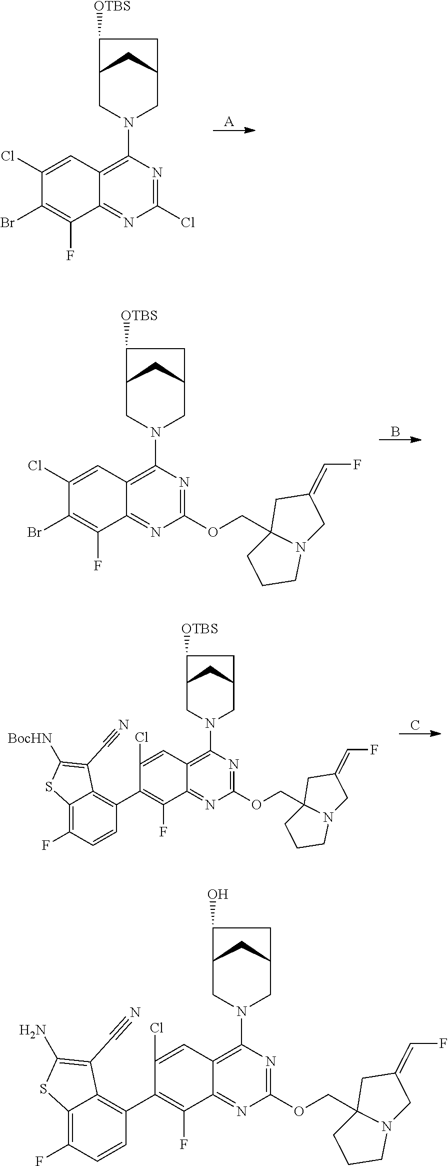

Step A. 7-bromo-4-((1R,5R,6R)-6-((tert-butyldimethylsilyl)oxy)-3-azabicyclo[3.2.1]octan-3-yl)-6-chloro-8-fluoro-2-(((Z)-2-(fluoromethylene)tetrahydro-1H-pyrrolizin-7a(5H)-yl)methoxy)quinazoline: To a mixture of 7-bromo-4-((1R,5R,6R)-6-((tert-butyldimethylsilyl)oxy)-3-azabicyclo[3.2.1]octan-3-yl)-2,6-dichloro-8-fluoroquinazoline (300 mg, 1.0 equiv), (Z)-(2-(fluoromethylene)tetrahydro-1H-pyrrolizin-7a(5H)-yl)methanol (125 mg, 1.3 equiv) and 1,4-diazabicyclo[2.2.2]octane (62.9 mg, 1.0 equiv) in DMF (1.0 mL) and THE (1.0 mL) was added Cs₂CO₃ (548 mg, 3.0 equiv). The reaction was stirred at 30° C. for 12 hours. The mixture was filtered and purified by reversed phase flash chromatography [C18, 0.1% formic acid condition] to afford the title compound (80.0 mg, 19% yield) as yellow solid; LCMS (ESI, M+1, M+3): m/z=669.2, 671.2.

Step B. tert-butyl (4-(4-((1R,5R,6R)-6-((tert-butyldimethylsilyl)oxy)-3-azabicyclo[3.2.1]octan-3-yl)-6-chloro-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a mixture of 7-bromo-4-((1R,5R,6R)-6-((tert-butyldimethylsilyl)oxy)-3-azabicyclo[3.2.1]octan-3-yl)-6-chloro-8-fluoro-2-(((Z)-2-(fluoromethylene)tetrahydro-1H-pyrrolizin-7a(5H)-yl)methoxy)quinazoline (60.0 mg, 1.0 equiv), tert-butyl (3-cyano-4-(5,5-dimethyl-1,3,2-dioxaborinan-2-yl)-7-fluorobenzo[b]thiophen-2-yl)carbamate (90.5 mg, 2.5 equiv) and Cs₂CO₃ (175 mg, 6.0 equiv) in THE (4 mL) was added Pd(DPEphos)Cl₂ (12.3 mg, 0.2 equiv). The reaction was degassed and purged with nitrogen 3 times. The reaction was stirred at 65° C. for 2 hours. The mixture was concentrated. The residue was diluted with water (2 mL) and extracted with EtOAc (3×2 mL). The combined organic layers were washed with brine (2 mL), dried over anhydrous sodium sulfate, concentrated, and purified by reversed phase flash chromatography [C18, 0.1% formic acid condition] to afford the title compound (80.0 mg, 50% yield) as yellow solid; LCMS (ESI, M+1): m/z=881.3.

Step C. 2-amino-4-(6-chloro-8-fluoro-2-(((Z)-2-(fluoromethylene)tetrahydro-1H-pyrrolizin-7a(5H)-yl)methoxy)-4-((1R,5R,6R)-6-hydroxy-3-azabicyclo[3.2.1]octan-3-yl)quinazolin-7-yl)-7-fluorobenzo[b]thiophene-3-carbonitrile: A solution of tert-butyl (4-(4-((1R,5R,6R)-6-((tert-butyldimethylsilyl)oxy)-3-azabicyclo[3.2.1]octan-3-yl)-6-chloro-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (60.0 mg, 1.0 equiv) in HCl.MeOH (4 M, 2 mL) was stirred at 25° C. for 6 hours. The mixture was concentrated. The residue was dilute with water (2 mL) and its pH was adjusted to 7 with NaHCO₃. The mixture was extracted with EtOAc (3×2 mL). The combined organic layers were washed with brine (3×2 mL), dried over anhydrous sodium sulfate, concentrated and purified by prep-HPLC [YMC-Actus Triart C18 150×30 mm×7 μm; A: water (FA); B: ACN, B: 25%-55% over 10 min] followed by prep-HPLC [Waters Xbridge 150×25 mm×5 μm; A: water (10 mM ammonia hydroxide), B: ACN, B %: 52%-82%% over 15 min] to afford the title compound (2.23 mg, 4.8% yield) as white solid; ¹H NMR (400 MHz, CHLOROFORM-d) δ=7.81-7.77 (m, 1H), 7.25-7.19 (m, 1H), 7.07-6.99 (m, 1H), 6.62-6.36 (m, 1H), 5.38 (s, 2H), 4.85 (d, J=3.2 Hz, 1H), 4.54-4.29 (m, 2H), 4.28-4.12 (m, 2H), 4.09-4.00 (m, 1H), 3.92-3.83 (m, 1H), 3.83-3.76 (m, 1H), 3.48-3.35 (m, 2H), 3.26-3.14 (m, 1H), 2.80-2.58 (m, 2H), 2.39-2.30 (m, 2H), 2.29-2.24 (m, 1H), 2.24-2.11 (m, 2H), 1.97-1.87 (m, 2H), 1.84-1.78 (m, 2H), 1.78-1.73 (m, 1H); LCMS (ESI, M+1): m/z=667.0.

### Example 128

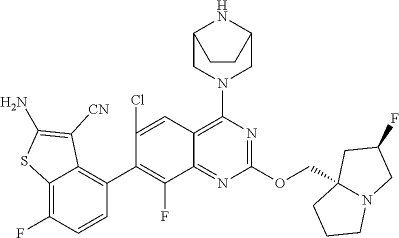 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

### 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

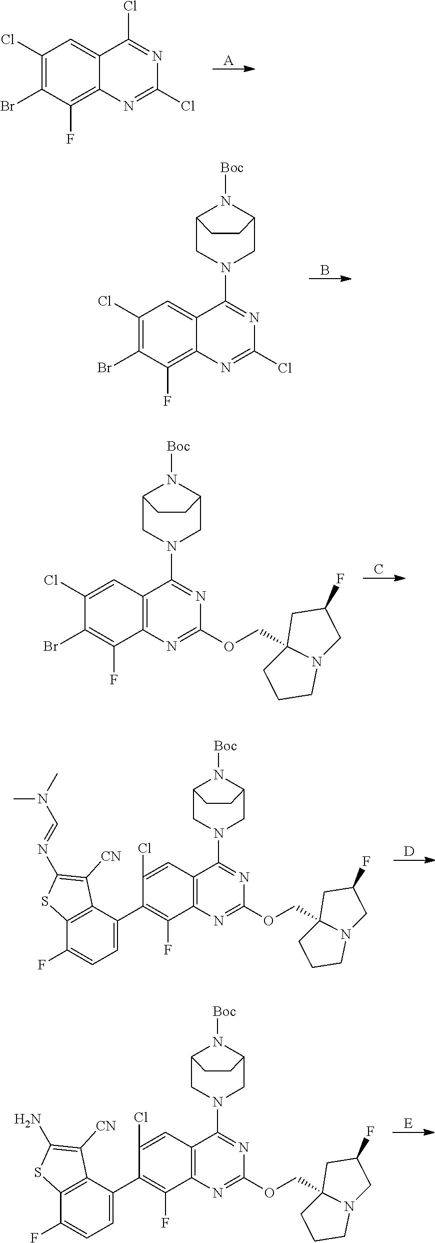

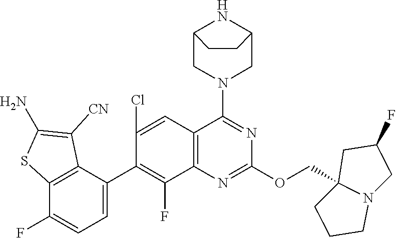 [SMILES: N#Cc1c(N)sc2c(F)ccc(-c3c(Cl)cc4c(N5CC6CCC(C5)N6)nc(OC[C@@]56CCCN5C[C@H](F)C6)nc4c3F)c12]

Step A. (1R,5S)-tert-butyl 3-(7-bromo-2,6-dichloro-8-fluoroquinazolin-4-yl)-3,8-diazabicyclo[3.2.1]octane-8-carboxylate: To a mixture of 7-bromo-2,4,6-trichloro-8-fluoroquinazoline (2.00 g, 6.05 mmol, 1.0 equiv) and DIEA (1.17 g, 1.5 equiv) in DCM (10 mL) was added and tert-butyl (1R,5S)-3,8-diazabicyclo[3.2.1]octane-8-carboxylate (1.41 g, 1.1 equiv). The reaction was stirred at −40° C. for 1 hour. The mixture was diluted with water (10 mL) and extracted with DCM (2×10 mL). The combined organic layers were washed with brine (2×10 mL), dried over anhydrous sodium sulfate, and concentrated to afford the title compound (3.00 g, 97% yield) as yellow solid; LCMS (ESI, M+1, M+3): m/z=505.1, 507.1.

Step B. (1R,5S)-tert-butyl 3-(7-bromo-6-chloro-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate: A mixture of (1R,5S)-tert-butyl 3-(7-bromo-2,6-dichloro-8-fluoroquinazolin-4-yl)-3,8-diazabicyclo[3.2.1]octane-8-carboxylate (1.0 g, 1.98 mmol, 1.0 equiv) and ((2R,7aS)-2-fluorohexahydro-1H-pyrrolizin-7a-yl)methanol (6.29 g, 39.5 mmol, 20 equiv) was stirred at 100° C. for 12 hours. The mixture was purified with prep-HPLC [Phenomenex luna C18 150×40 mm×15 μm; A: water (0.1% FA), B: ACN, B %: 22%-52% over 11 min] to afford the title compound (715 mg, 52% yield) as yellow solid; LCMS (ESI, M+1, M+3): m/z=628.4, 630.4.

Step C. (1R,5S)-tert-butyl 3-(6-chloro-7-(3-cyano-2-((E)-((dimethylamino)methylene)amino)-7-fluorobenzo[b]thiophen-4-yl)-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate: To a mixture of (1R,5S)-tert-butyl 3-(7-bromo-6-chloro-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate (300 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (534 mg, 3.0 equiv) and aqueous K₃PO₄ (1.3 mL, 1.5 M) in methoxycyclopentane (5 mL) was added APhos Pd G3 (30.3 mg, 0.10 equiv). The reaction was degassed and purged with nitrogen 3 times. The reaction was stirred at 60° C. for 2 hours. The mixture was quenched by H₂O (5 mL) and extracted with EtOAc (2×5 mL). The combined organic layers were washed with brine (2×5 mL), dried over anhydrous sodium sulfate, and concentrated to afford the title compound (753 mg, crude) as yellow solid.

Step D. (1R,5S)-tert-butyl 3-(7-(2-amino-3-cyano-7-fluorobenzo[b]thiophen-4-yl)-6-chloro-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate: To a solution of (1R,5S)-tert-butyl 3-(6-chloro-7-(3-cyano-2-((E)-((dimethylamino)methylene)amino)-7-fluorobenzo[b]thiophen-4-yl)-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate (700 mg, 1.0 equiv.) in DMAc (10 mL) was added aqueous K₃PO₄ (2 mL, 1.5 M). The reaction was stirred at 80° C. for 10 hours. The mixture was quenched with H₂O (10 mL) and extracted with EtOAc (2×10 mL). The combined organic layers were washed with brine (2×5 mL), dried over anhydrous sodium sulfate, concentrated, and purified by Prep-HPLC[Phenomenex luna C18 150×40 mm×15 μm; A: water (0.1% FA), B: ACN, B %: 25%-55% over 11 min) to afford the title compound (72.0 mg, 23% yield) as yellow solid; LCMS (ESI, M+1): m/z=740.2.

Step E. 4-(4-((1R,5S)-3,8-diazabicyclo[3.2.1]octan-3-yl)-6-chloro-8-fluoro-2-(((2R,7aS)-2-fluorohexahydro-1H-pyrrolizin-7a-yl)methoxy)quinazolin-7-yl)-2-amino-7-fluorobenzo[b]thiophene-3-carbonitrile: A mixture of (1R,5S)-tert-butyl 3-(7-(2-amino-3-cyano-7-fluorobenzo[b]thiophen-4-yl)-6-chloro-8-fluoro-2-(((2R,7aS)-2-fluorohexahydro-1H-pyrrolizin-7a-yl)methoxy)quinazolin-4-yl)-3,8-diazabicyclo[3.2.1]octane-8-carboxylate (72.0 mg, 1.0 equiv) and HCl.dioxane (4 M, 3.60 mL) was stirred at 25° C. for 1 hour. The mixture was concentrated under vacuum and purified by Prep-HPLC[C18 150×25 mm×10 μm; A: water (0.1% FA), B: ACN, B %: 10%-40% over 11 min] to afford the title compound (4.30 mg, 6.7% yield, HCOOH) as yellow solid; ¹H NMR (400 MHz, DMSO-d₆) δ=8.16 (s, 1H), 8.10 (s, 2H), 7.84 (s, 1H), 7.28-7.22 (m, 1H), 7.18-7.11 (m, 1H), 5.35-5.18 (m, 1H), 4.37-4.21 (m, 2H), 4.10-4.05 (m, 1H), 4.01-3.95 (m, 1H), 3.61-3.56 (m, 3H), 3.51 (br d, J=12.0 Hz, 1H), 3.11-3.05 (m, 2H), 3.00 (br d, J=1.2 Hz, 1H), 2.86-2.77 (m, 1H), 2.16-2.10 (m, 1H), 2.09-1.95 (m, 2H), 1.86-1.73 (m, 3H), 1.70-1.56 (m, 4H); LCMS (ESI, M+1): m/z=640.3.

### Example 129

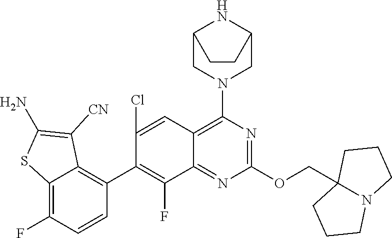 [SMILES: N#Cc1c(N)sc2c(F)ccc(-c3c(Cl)cc4c(N5CC6CCC(C5)N6)nc(OCC56CCCN5CCC6)nc4c3F)c12]

### 4-(4-((1R,5S)-3,8-diazabicyclo[3.2.1]octan-3-yl)-6-chloro-8-fluoro-2-((tetrahydro-1H-pyrrolizin-7a(5H)-yl)methoxy)quinazolin-7-yl)-2-amino-7-fluorobenzo[b]thiophene-3-carbonitrile

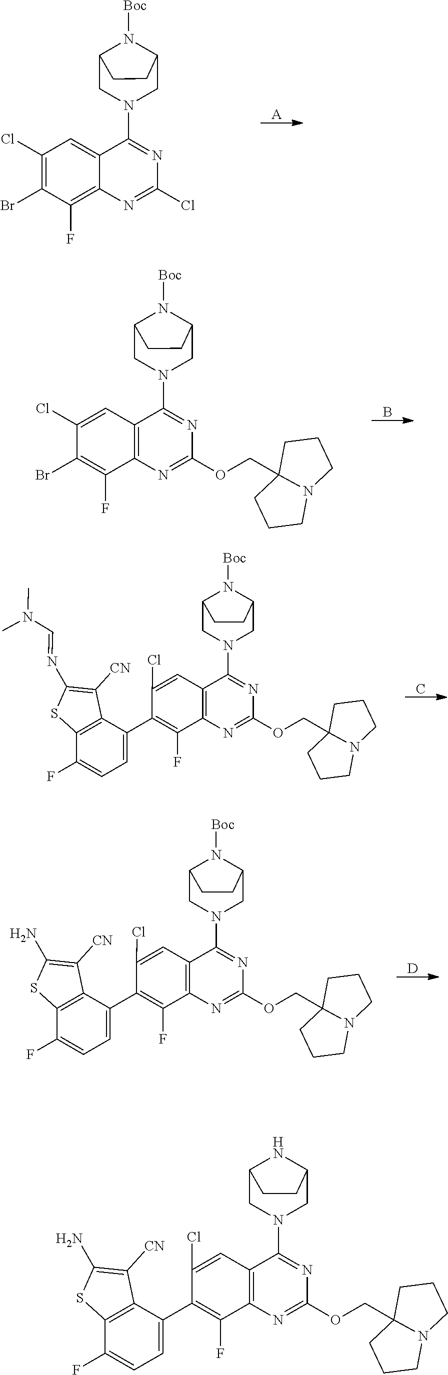

Step A. (1R,5S)-tert-butyl 3-(7-bromo-6-chloro-8-fluoro-2-((hexahydro-1H-pyrrolizin-7a-yl)methoxy)quinazolin-4-yl)-3,8-diazabicyclo[3.2.1]octane-8-carboxylate: A mixture of (1R,5S)-tert-butyl 3-(7-bromo-2,6-dichloro-8-fluoroquinazolin-4-yl)-3,8-diazabicyclo[3.2.1]octane-8-carboxylate (1.00 g, 1.0 equiv.) and (hexahydro-1H-pyrrolizin-7a-yl)methanol (5.58 g, 20 equiv) was stirred at 60° C. for 2 hours. The mixture was filtered and purified by Prep-HPLC [Phenomenex luna C18 150×40 mm×15 μm; A: water (0.1% FA); B: ACN, B %: 22%-52% over 11 min] to afford the title compound (630 mg, 46% yield) as yellow solid; LCMS (ESI, M+1, M+3): m/z=610.4, 612.4.

Step B. (1R,5S)-tert-butyl 3-(6-chloro-7-(3-cyano-2-((E)-((dimethylamino)methylene)amino)-7-fluorobenzo[b]thiophen-4-yl)-8-fluoro-2-((hexahydro-1H-pyrrolizin-7a-yl)methoxy)quinazolin-4-yl)-3,8-diazabicyclo[3.2.1]octane-8-carboxylate: To a mixture of (1R,5S)-tert-butyl 3-(7-bromo-6-chloro-8-fluoro-2-((hexahydro-1H-pyrrolizin-7a-yl)methoxy)quinazolin-4-yl)-3,8-diazabicyclo[3.2.1]octane-8-carboxylate (630 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (550 mg, 3.0 equiv) and aqueous K₃PO₄ (0.5 mL, 1.5 M) in methoxycyclopentane (5 mL) was added APhos Pd G3 (31.2 mg, 0.10 equiv). The reaction was degassed and purged with nitrogen 3 times. The reaction was stirred at 60° C. for 2 hours. The mixture was quenched by H₂O (5 mL) and extracted with EtOAc (2×5 mL). The combined organic layers were washed with brine, dried over anhydrous sodium sulfate, and concentrated to afford the title compound (800 mg, 94% yield) as yellow solid.

Step C. (1R,5S)-tert-butyl 3-(7-(2-amino-3-cyano-7-fluorobenzo[b]thiophen-4-yl)-6-chloro-8-fluoro-2-((hexahydro-1H-pyrrolizin-7a-yl)methoxy)quinazolin-4-yl)-3,8-diazabicyclo[3.2.1]octane-8-carboxylate: To a solution of (1R,5S)-tert-butyl 3-(6-chloro-7-(3-cyano-2-((E)-((dimethylamino)methylene)amino)-7-fluorobenzo[b]thiophen-4-yl)-8-fluoro-2-((hexahydro-1H-pyrrolizin-7a-yl)methoxy)quinazolin-4-yl)-3,8-diazabicyclo[3.2.1]octane-8-carboxylate (800 mg, 1.0 equiv) in DMAc (10 mL) was added aqueous K₃PO₄ (0.1 mL, 1.5 M). The reaction was stirred at 80° C. for 10 hours. The mixture was quenched by H₂O (10 mL) and extracted with EtOAc (2×10 mL). The combined organic layers were washed with brine (20 mL), dried over anhydrous sodium sulfate, concentrated and purified by Prep-HPLC [Phenomenex luna 150×40 mm×15 μm; A: water (0.1% FA); B: ACN, B %: 25%-55% over 11 min] to afford the title compound as yellow solid (55.0 mg, 15% yield); LCMS (ESI, M+1): m/z=722.2.

Step D. 4-(4-((1R,5S)-3,8-diazabicyclo[3.2.1]octan-3-yl)-6-chloro-8-fluoro-2-((hexahydro-1H-pyrrolizin-7a-yl)methoxy)quinazolin-7-yl)-2-amino-7-fluorobenzo[b]thiophene-3-carbonitrile: A solution of (1R,5S)-tert-butyl 3-(7-(2-amino-3-cyano-7-fluorobenzo[b]thiophen-4-yl)-6-chloro-8-fluoro-2-((hexahydro-1H-pyrrolizin-7a-yl)methoxy)quinazolin-4-yl)-3,8-diazabicyclo[3.2.1]octane-8-carboxylate (55.0 mg, 1.0 equiv) in HCl.dioxane (4 M, 2.75 mL) was stirred at 25° C. for 1 hour. The reaction was filtered and purified by Prep-HPLC [Phenomenex luna 150×25 mm×10 μm; A: water (0.1% FA); B: ACN, B %: 10%-40% over 9 min] to afford the title compound (10.4 mg, 21% yield, 1.2 HCOOH) as white solid; ¹H NMR (400 MHz, DMSO-d6) δ=8.20 (s, 1H), 8.10 (s, 2H), 7.85 (s, 1H), 7.26 (dd, J=8.4 Hz, 1H), 7.18-7.11 (m, 1H), 4.36-4.21 (m, 2H), 4.08 (s, 2H), 3.58 (br s, 3H), 3.04-2.97 (m, 2H), 2.66-2.60 (m, 3H), 1.93 (td, J=6.0, 12.0 Hz, 2H), 1.87-1.74 (m, 4H), 1.69-1.58 (m, 6H); LCMS (ESI, M+1): m/z=622.4.

### Example 130

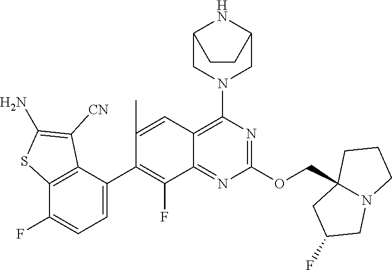 [SMILES: Cc1cc2c(N3CC4CCC(C3)N4)nc(OC[C@@]34CCCN3C[C@H](F)C4)nc2c(F)c1-c1ccc(F)c2sc(N)c(C#N)c12]

### 4-(4-((1R,5S)-3,8-diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-2-amino-7-fluorobenzo[b]thiophene-3-carbonitrile

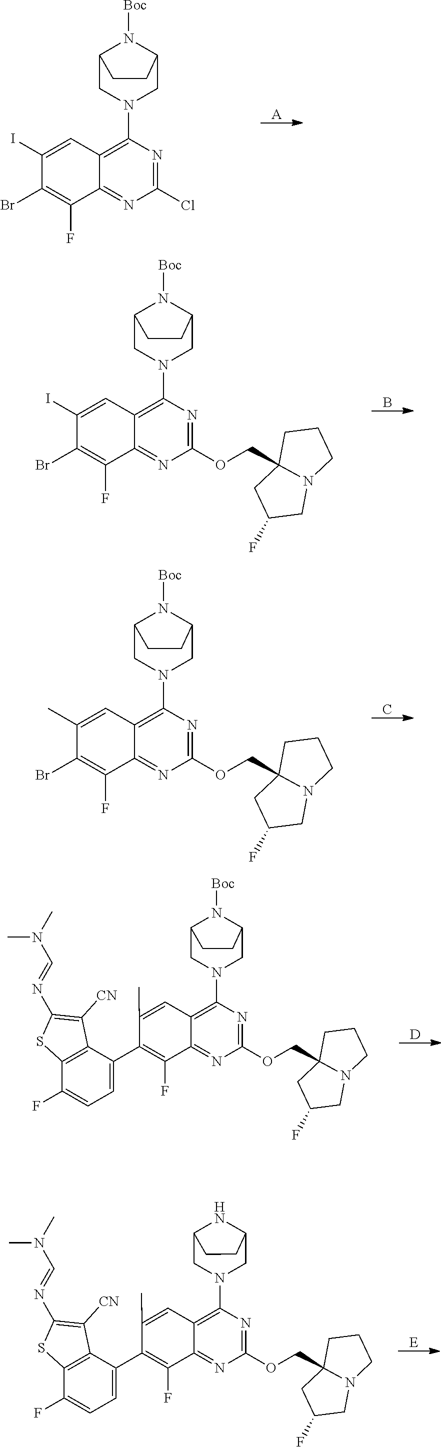

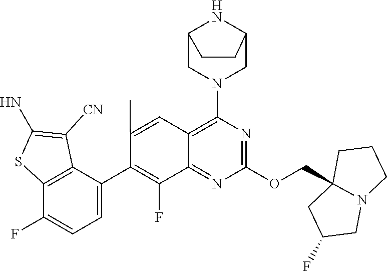 [SMILES: Cc1cc2c(N3CC4CCC(C3)N4)nc(OC[C@@]34CCCN3C[C@H](F)C4)nc2c(F)c1-c1ccc(F)c2sc(N)c(C#N)c12]

Step A. tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate: A mixture of tert-butyl (1R,5S)-3-(7-bromo-2-chloro-8-fluoro-6-iodoquinazolin-4-yl)-3,8-diazabicyclo[3.2.1]octane-8-carboxylate (968 mg, 1.0 equiv, synthesized according to example 124 step A) and ((2R,7aS)-2-fluorotetrahydro-1H-pyrrolizin-7a(5H)-yl)methanol (2.58 g, 10 equiv) was stirred at 100° C. for 30 hours. The mixture was quenched with H₂O (150 mL) and extracted with EtOAc (3×25 mL). The combined organic layers were washed with brine (200 mL), dried over anhydrous sodium sulfate, concentrated, and purified with reversed phase flash chromatography[C18, 0.1% formic acid condition] to afford title compound (260 mg, 21% yield) as yellow solid; LCMS (ESI, M+1, M+3): m/z=720.1, 722.1.

Step B. tert-butyl (1R,5S)-3-(7-bromo-8-fluoro-2-(((2R,7aS)-2-fluorotetrahydro-1H-pyrrolizin-7a(5H)-yl)methoxy)-6-methylquinazolin-4-yl)-3,8-diazabicyclo[3.2.1]octane-8-carboxylate: To a mixture of tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate (370 mg, 1 equiv), 2,4,6-trimethyl-1,3,5,2,4,6-trioxatriborinane (3.5 M, 3 eq), K₂CO₃ aqueous (1.5 M, 1.03 mL, 3 equiv) in dioxane (6 mL) was added Pd(dppf)Cl₂ (37.6 mg, 0.1 equiv). The reaction was degassed and purged with nitrogen 3 times. The reaction was stirred at 50° C. for 12 hours. The mixture was diluted with water (20 mL) and extracted with ethyl acetate (2×20 mL). The combined organic layers were concentrated and purified with reversed phase flash chromatography [C18, 0.1% formic acid condition] to afford title compound (140 mg, 44.79% yield) as a yellow solid.

Step C. tert-butyl (1R,5S)-3-(7-(3-cyano-2-(((E)-(dimethylamino)methylene)amino)-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl)-3,8-diazabicyclo[3.2.1]octane-8-carboxylate: To a mixture of tert-butyl (1R,5S)-3-(7-bromo-8-fluoro-2-(((2R,7aS)-2-fluorotetrahydro-1H-pyrrolizin-7a(5H)-yl)methoxy)-6-methylquinazolin-4-yl)-3,8-diazabicyclo[3.2.1]octane-8-carboxylate (100 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (92.0 mg, 1.5 eq), aqueous K₃PO₄ (1.5 M, 438 μL, 4.0 equiv) in THE (1 mL) was added RuPhos Pd G3 (13.74 mg, 0.1 eq). The reaction was degassed and purged with nitrogen 3 times. The reaction was stirred at 60° C. for 12 hours. The mixture was diluted with water (20 ml) and extracted with EtOAc (2×30 ml). The combined organic layers were washed with brine(10 mL), dried over anhydrous sodium sulfate, concentrated and purified with reversed phase flash chromatography[C18, 0.1% formic acid condition] to afford title compound (110 mg, 86% yield) as brown solid; LCMS (ESI, M+1): m/z=775.4.

Step D. (E)-N′-(4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)-N,N-dimethylformimidamide: A mixture of tert-butyl (1R,5S)-3-(7-(3-cyano-2-(((E)-(dimethylamino)methylene)amino)-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl)-3,8-diazabicyclo[3.2.1]octane-8-carboxylate (220 mg, 1.0 equiv) and HCl.MeOH (4 M, 2 mL, 28 equiv) was stirred at 25° C. for 0.5 hours. The mixture was concentrated, basified with NaHCO₃ aqueous (10 ml) and extracted with EtOAc (2×10 ml). The combined organic layers were washed with brine (10 mL), dried over anhydrous sodium sulfate and concentrated to afford title compound (130 mg, crude) as brown solid; LCMS (ESI, M+1): m/z=675.3.

Step E. 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-2-amino-7-fluorobenzo[b]thiophene-3-carbonitrile: To a mixture of (E)-N′-(4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)-N,N-dimethylformimidamide (120 mg, 1.0 equiv) in DMAC (2 mL) was added aqueous K₃PO₄ (1.5 M, 2.37 mL, 20 equiv). The reaction was stirred at 80° C. for 0.5 hours. The mixture was diluted with water (5 ml) and extracted with EtOAc (2×5 mL). The combined organic layers were washed with brine (5 mL), dried over anhydrous sodium sulfate, concentrated and purified by prep-HPLC [Phenomenex luna C18 150×25 mm×10 μm; A: water (FA), B: ACN, B %: 5%-35% over 10 min] to afford the title compound (73.4 mg, 59% yield, 1.23 HCOOH) as yellow solid; ¹H NMR (400 MHz, METHANOL-d₄) δ=7.67 (s, 1H), 7.17-7.14 (m, 1H), 7.06-7.02 (m, 1H), 5.44 (d, J=52.8 Hz, 1H), 4.60-4.55 (m, 2H), 4.52-4.41 (m, 2H), 3.98 (br s, 2H), 3.77-3.59 (m, 5H), 3.31-2.26 (m, 1H), 2.62-2.41 (m, 2H), 2.40-2.25 (m, 1H), 2.20-2.18 (m, 5H), 2.03-1.85 (m, 5H); LCMS (ESI, M+1): m/z=620.3.

### Example 131

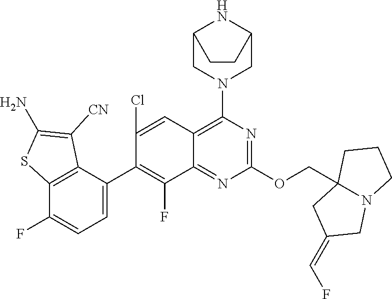 [SMILES: N#Cc1c(N)sc2c(F)ccc(-c3c(Cl)cc4c(N5CC6CCC(C5)N6)nc(OCC56CCCN5C/C(=C\F)C6)nc4c3F)c12]

### 4-(4-((1R,5S)-3,8-diazabicyclo[3.2.1]octan-3-yl)-6-chloro-8-fluoro-2-(((Z)-2-(fluoromethylene)tetrahydro-1H-pyrrolizin-7a(5H)-yl)methoxy)quinazolin-7-yl)-2-amino-7-fluorobenzo[b]thiophene-3-carbonitrile

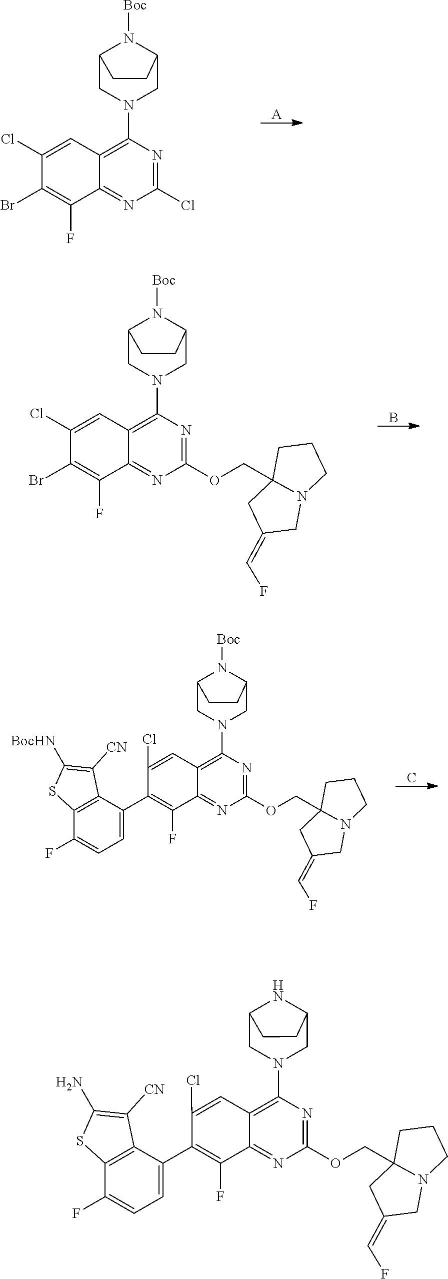

Step A. tert-butyl (1R,5S)-3-(7-bromo-6-chloro-8-fluoro-2-(((Z)-2-(fluoromethylene)tetrahydro-1H-pyrrolizin-7a(5H)-yl)methoxy)guinazolin-4-yl)-3,8-diazabicyclo[3.2.1]octane-8-carboxylate: To a mixture of tert-butyl (1R,5S)-3-(7-bromo-2,6-dichloro-8-fluoroquinazolin-4-yl)-3,8-diazabicyclo[3.2.1]octane-8-carboxylate (400 mg, 1.0 equiv), 1,4-diazabicyclo[2.2.2]octane (88.6 mg, 1.0 equiv) and (Z)-(2-(fluoromethylene)tetrahydro-1H-pyrrolizin-7a(5H)-yl)methanol (203 mg, 1.5 equiv) in THF (3 mL) and DMF (3 mL) was added Cs₂CO₃ (772 mg, 3.0 equiv). The reaction was stirred at 30° C. for 12 hours. The mixture was diluted with water (10 mL) and extracted with ethyl acetate (2×10 mL). The combined organic layers were dried over anhydrous sodium sulfate, concentrated and purified by reversed phase flash chromatography [C18, 0.1% formic acid condition] to afford the title compound (110 mg, 21% yield) as yellow solid; LCMS (ESI, M+1, M+3): m/z=640.2, 642.2.

Step B. tert-butyl (1R,5S)-3-(7-(2-((tert-butoxycarbonyl)amino)-3-cyano-7-fluorobenzo[b]thiophen-4-yl)-6-chloro-8-fluoro-2-(((Z)-2-(fluoromethylene)tetrahydro-1H-pyrrolizin-7a(5H)-yl)methoxy)quinazolin-4-yl)-3,8-diazabicyclo[3.2.1]octane-8-carboxylate: To a mixture of tert-butyl (1R,5S)-3-(7-bromo-6-chloro-8-fluoro-2-(((Z)-2-(fluoromethylene)tetrahydro-1H-pyrrolizin-7a(5H)-yl)methoxy)quinazolin-4-yl)-3,8-diazabicyclo[3.2.1]octane-8-carboxylate (110 mg, 1.0 equiv), tert-butyl (3-cyano-4-(5,5-dimethyl-1,3,2-dioxaborinan-2-yl)-7-fluorobenzo[b]thiophen-2-yl)carbamate (173 mg, 2.5 equiv), Cs₂CO₃ (356 mg, 6.0 equiv) in THE (5 mL) was added Pd(DPEphos)Cl₂ (23.6 mg, 0.2 equiv). The reaction was stirred at 65° C. for 2 hours under N₂ atmosphere. The mixture was filtered and concentrated. The residue was diluted with water (2 mL) and extracted with ethyl acetate (3×2 mL). The combined organic layers were washed with brine (2 mL), dried over anhydrous sodium sulfate, concentrated and purified by reversed phase flash chromatography [C18, 0.1% formic acid condition] to afford the title compound (120 mg, 70% yield) as yellow solid; LCMS (ESI, M+1): m/z=852.3.

Step C. 4-(4-((1R,5S)-3,8-diazabicyclo[3.2.1]octan-3-yl)-6-chloro-8-fluoro-2-(((Z)-2-(fluoromethylene)tetrahydro-1H-pyrrolizin-7a(5H)-yl)methoxy)guinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-6-chloro-8-fluoro-2-(((Z)-2-(fluoromethylene)tetrahydro-1H-pyrrolizin-7a(5H)-yl)methoxy)quinazolin-4-yl)-3,8-diazabicyclo[3.2.1]octane-8-carboxylate (110 mg, 1.0 equiv) in DCM (2 mL) was added TFA (3.07 g, 208 equiv). The reaction was stirred at 25° C. for 0.5 hour. The mixture was concentrated. The residue was dilute with water (2 mL) and neutralized with NaHCO₃, extracted with ethyl acetate (3×2 mL). The combined organic layers were washed with brine (3×2 mL), dried over anhydrous sodium sulfate, concentrated and purified with prep-HPLC [YMC Triart C18 150×25 mm×5 μm; A: water (10 mM formic acid); B: ACN, B %: 10%-40% over 10 min] to afford the title compound (19.5 mg, 22% yield, 0.50 HCOOH) as white solid; ¹H NMR (400 MHz, METHANOL-d₄) δ=7.93-7.85 (m, 1H), 7.25-7.17 (m, 1H), 7.04 (t, J=8.8 Hz, 1H), 6.85-6.57 (m, 1H), 4.64-4.50 (m, 2H), 4.49-4.34 (m, 2H), 4.11-4.00 (m, 1H), 3.97-3.84 (m, 2H), 3.79-3.65 (m, 3H), 3.42-3.34 (m, 1H), 2.97-2.87 (m, 1H), 2.86-2.78 (m, 1H), 2.60-2.52 (m, 1H), 2.27-2.17 (m, 1H), 2.13-2.05 (m, 1H), 2.03-1.91 (m, 6H); LCMS (ESI, M+1): m/z=652.3.

### Example 132

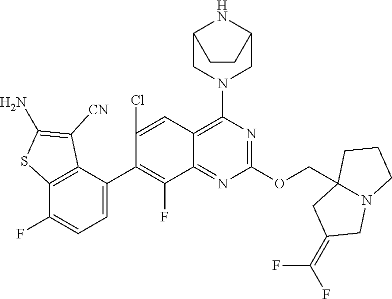 [SMILES: N#Cc1c(N)sc2c(F)ccc(-c3c(Cl)cc4c(N5CC6CCC(C5)N6)nc(OCC56CCCN5CC(=C(F)F)C6)nc4c3F)c12]

### 4-(4-((1R,5S)-3,8-diazabicyclo[3.2.1]octan-3-yl)-6-chloro-2-((2-(difluoromethylene)tetrahydro-1H-pyrrolizin-7a(5H)-yl)methoxy)-8-fluoroquinazolin-7-yl)-2-amino-7-fluorobenzo[b]thiophene-3-carbonitrile

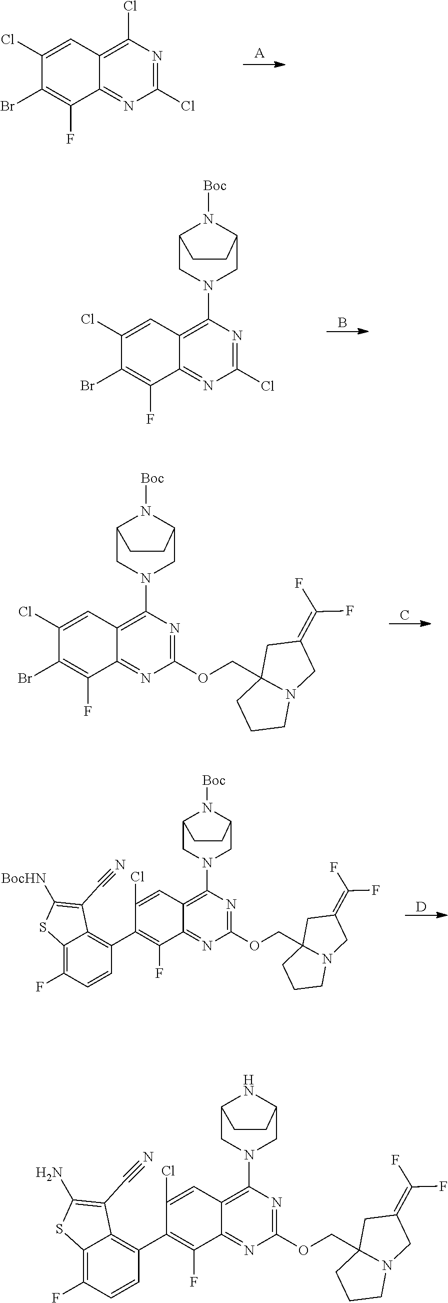

Step A. tert-butyl (1R,5S)-3-(7-bromo-2,6-dichloro-8-fluoroquinazolin-4-yl)-3,8-diazabicyclo[3.2.1]octane-8-carboxylate: To a solution of 7-bromo-2,4,6-trichloro-8-fluoroquinazoline (2.50 g, 1.0 equiv) and TEA (2.30 g, 3.0 equiv) in DCM (80.0 mL) was slowly added tert-butyl (1R,5S)-3,8-diazabicyclo[3.2.1]octane-8-carboxylate (1.69 g, 1.05 equiv) at −40° C. The mixture was stirred at −40° C. for 1 hour. The mixture was quenched with water (200 mL) and extracted with dichloromethane (2×100 mL). The combined organic layers were dried over anhydrous sodium sulfate, concentrated to afford the title compound (3.90 g, crude) as yellow solid. LCMS (ESI, M+1, M+3): m/z=504.8. 506.8.

Step B. tert-butyl (1R,5S)-3-(7-bromo-6-chloro-2-((2-(difluoromethylene)tetrahydro-1H-pyrrolizin-7a(5H)-yl)methoxy)-8-fluoroquinazolin-4-yl)-3,8-diazabicyclo[3.2.1]octane-8-carboxylate: To a mixture of tert-butyl (1R,5S)-3-(7-bromo-2,6-dichloro-8-fluoroquinazolin-4-yl)-3,8-diazabicyclo[3.2.1]octane-8-carboxylate (230 mg, 1.0 equiv), (2-(difluoromethylene)tetrahydro-1H-pyrrolizin-7a(5H)-yl)methanol (103 mg, 1.2 equiv) and Cs₂CO₃ (444 mg, 3.0 equiv) in DMF (5.00 mL) and THE (5.00 mL) was added DABCO (51.0 mg, 1.0 equiv). The reaction was stirred at 25° C. for 3 hours. The mixture was diluted with water (20 mL) and extracted with ethyl acetate (2×10 mL). The combined organic layers were dried over anhydrous sodium sulfate, concentrated and purified by reversed phase flash chromatography [C18, 0.1% formic acid condition] to afford the title compound (66 mg, 20.7% yield) as yellow solid; LCMS (ESI, M+1, M+3): m/z=657.9 659.9.

Step C. tert-butyl (1R,5S)-3-(7-(2-((tert-butoxycarbonyl)amino)-3-cyano-7-fluorobenzo[b]thiophen-4-yl)-6-chloro-2-((2-(difluoromethylene)tetrahydro-1H-pyrrolizin-7a(5H)-yl)methoxy)-8-fluoroquinazolin-4-yl)-3,8-diazabicyclo[3.2.1]octane-8-carboxylate: To a solution of tert-butyl (1R,5S)-3-(7-bromo-6-chloro-2-((2-(difluoromethylene)tetrahydro-1H-pyrrolizin-7a(5H)-yl)methoxy)-8-fluoroquinazolin-4-yl)-3,8-diazabicyclo[3.2.1]octane-8-carboxylate (60.0 mg, 1.0 equiv) and tert-butyl (3-cyano-4-(5,5-dimethyl-1,3,2-dioxaborinan-2-yl)-7-fluorobenzo[b]thiophen-2-yl)carbamate (73.6 mg, 2.0 equiv) in toluene (2.00 mL) were added Pd(DPEphos)Cl₂ (9.39 mg, 0.15 equiv) and Cs₂CO₃ (89.0 mg, 3.0 equiv). The reaction was degassed and purged with N₂ for 3 times. The mixture was stirred at 110° C. for 1 hour. The mixture was diluted with water (20 mL) and extracted with EtOAc (2×10 mL). The combined organic layers were washed with brine (20 mL), dried over anhydrous sodium sulfate, filtered and concentrated and purified by reversed phase flash chromatography [C18, 0.1% formic acid condition] to afford the title compound (40 mg, 37% yield) as yellow solid; LCMS (ESI, M+1): m/z=870.2.

Step D. 4-(4-((1R,5S)-3,8-diazabicyclo[3.2.1]octan-3-yl)-6-chloro-2-((2-(difluoromethylene)tetrahydro-1H-pyrrolizin-7a(5H)-yl)methoxy)-8-fluoroquinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-6-chloro-2-((2-(difluoromethylene)tetrahydro-1H-pyrrolizin-7a(5H)-yl)methoxy)-8-fluoroquinazolin-4-yl)-3,8-diazabicyclo[3.2.1]octane-8-carboxylate (35.0 mg, 1.0 equiv) in DCM (2.00 mL) was added TFA (3.07 g, 669 equiv). The reaction was stirred at 10° C. for 0.5 hours. The mixture was concentrated, and purified with prep-HPLC [column: Waters Xbridge 150×25 mm×5 μm; A: water(NH₄HCO₃), B:ACN; B %:37%-67% over 8 min] to afford the title compound (5.91 mg, 22% yield) as white solid; ¹H NMR (400 MHz, METHANOL-d₄) δ=7.86 (d, J=1.6 Hz, 1H), 7.23-7.18 (m, 1H), 7.03 (dd, J=8.5, 9.4 Hz, 1H), 4.58-4.48 (m, 1H), 4.42 (br d, J=11.9 Hz, 1H), 4.34-4.30 (m, 1H), 4.27-4.20 (m, 1H), 3.79 (br d, J=14.3 Hz, 1H), 3.68-3.56 (m, 4H), 3.45-3.37 (m, 1H), 3.18-3.10 (m, 1H), 2.85-2.76 (m, 1H), 2.73-2.64 (m, 1H), 2.50 (br d, J=15.9 Hz, 1H), 2.21-2.07 (m, 1H), 2.04-1.96 (m, 1H), 1.96-1.87 (m, 2H), 1.86-1.78 (m, 4H); LCMS (ESI, M+1): m/z=670.2.

### Example 133

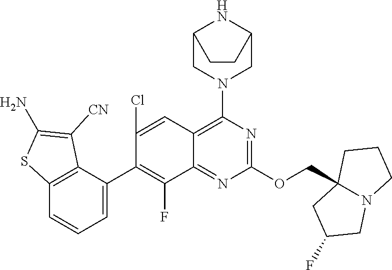 [SMILES: N#Cc1c(N)sc2cccc(-c3c(Cl)cc4c(N5CC6CCC(C5)N6)nc(OC[C@@]56CCCN5C[C@H](F)C6)nc4c3F)c12]

### 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl)methoxy)quinazolin-7-yl)-2-aminobenzo[b]thiophene-3-carbonitrile

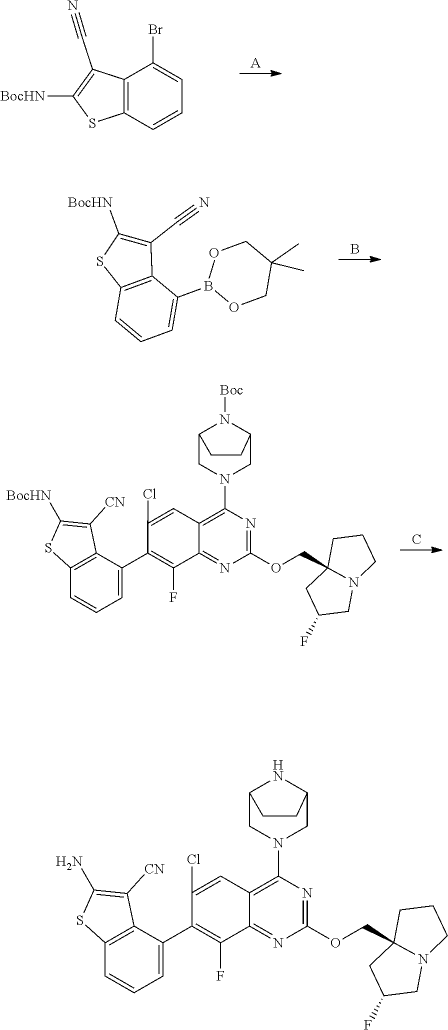

Step A. tert-butyl (3-cyano-4-(5,5-dimethyl-1,3,2-dioxaborinan-2-yl)benzo[b]thiophen-2-yl) carbamate: To a mixture of tert-butyl N-(4-bromo-3-cyano-benzothiophen-2-yl)carbamate (0.10 g, 1.0 equiv) and 2-(5,5-dimethyl-1,3,2-dioxaborinan-2-yl)-5,5-dimethyl-1,3,2-dioxaborinane (160 mg, 2.5 equiv) in dioxane (2 mL) were added Pd(DPEphos)Cl₂ (19.5 mg, 0.1 equiv) and potassium acetate (83.4 mg, 3.0 equiv). The reaction was degassed and purged with nitrogen for 3 times. The reaction was stirred at 105° C. for 1 hour under nitrogen atmosphere. The mixture was quenched with water (20 mL) at 20° C. and extracted with EtOAc (2×30 mL). The combined organic layers were washed with brine (30 mL), dried over anhydrous sodium sulfate and concentrated and purified with prep-TLC (SiO₂, PE: EA=3:1) to afford the title compound (50.0 mg, 46% yield) as light yellow solid; ¹H NMR (400 MHz, DMSO-d₆) δ=11.26 (s, 1H), 7.94 (dd, J=1.2, 8.0 Hz, 1H), 7.53 (dd, J=0.8, 6.8 Hz, 1H), 7.35-7.24 (m, 1H), 3.77 (s, 4H), 1.52 (s, 9H), 1.03 (s, 6H).

Step B. tert-butyl (1R,5S)-3-(7-(2-((tert-butoxycarbonyl)amino)-3-cyanobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-bromo-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (325 mg, 1.0 equiv) and tert-butyl N-[3-cyano-4-(5,5-dimethyl-1,3,2-dioxaborinan-2-yl)benzothiophen-2-yl]carbamate (499 mg, 2.5 equiv) in toluene (17 mL) were added Pd(DPEphos)Cl₂ (53.3 mg, 0.15 equiv) and Cs₂CO₃ (505 mg, 3.0 equiv). The reaction was degassed and purged with nitrogen for 3 times. The reaction was stirred at 110° C. for 4 hours under nitrogen atmosphere. The mixture was quenched with water (60 mL) at 20° C. and extracted with EtOAc (2×100 mL). The combined organic layers were washed with brine (60 mL), dried over anhydrous sodium sulfate and concentrated and purified with prep-HPLC (Phenomenex luna C18 150*40 mm*15 um; mobile phase: [water(FA)−ACN]; gradient:30%-60% B over 15 min) to afford the title compound (31.0 mg, 6.3% yield) as yellow solid; LCMS (ESI, M+1): m/z=822.3.

Step C. 4-(4-((1R,5S)-3,8-diazabicyclo[3.2.1]octan-3-yl)-6-chloro-8-fluoro-2-(((2R,7aS)-2-

fluorotetrahydro-1H-pyrrolizin-7a(5H)-yl)methoxy)quinazolin-7-yl)-2-aminobenzo[b]thiophene-3-carbonitrile: To a solution of tert-butyl (1R,5S)-3-(7-(2-((tert-butoxycarbonyl)amino)-3-cyanobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (28.0 mg, 1.0 equiv) in DCM (4 mL) was added TFA (3.07 g, 790 equiv). The reaction was stirred at 20° C. for 1 hour. The mixture was concentrated and purified with prep-HPLC (Phenomenex luna C18 150*25 mm*10 um; mobile phase: [water(FA)−ACN];gradient:10%-40% B over 7 min) to afford the title compound (8.69 mg, 37% yield, HCOOH) as white solid; ¹H NMR (400 MHz, DMSO-d₆) δ=8.20 (s, 1H), 7.86-7.75 (m, 4H), 7.27-7.21 (m, 1H), 7.19-7.15 (m, 1H), 5.27 (br d, J=54.0 Hz, 1H), 4.41-4.23 (m, 2H), 4.13-4.05 (m, 1H), 4.02-3.97 (m, 1H), 3.71-3.61 (m, 3H), 3.55 (br d, J=12.4 Hz, 1H), 3.15-3.05 (m, 2H), 2.87-2.78 (m, 1H), 2.15-1.97 (m, 3H), 1.91-1.56 (m, 8H); LCMS (ESI, M+1): m/z=622.1.

### Example 134

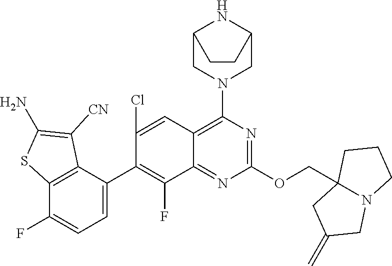 [SMILES: C=C1CN2CCCC2(COc2nc(N3CC4CCC(C3)N4)c3cc(Cl)c(-c4ccc(F)c5sc(N)c(C#N)c45)c(F)c3n2)C1]

### 4-(4-((1R,5S)-3,8-diazabicyclo[3.2.1]octan-3-yl)-6-chloro-8-fluoro-2-((2-methylenetetrahydro-1H-pyrrolizin-7a(5H)-yl)methoxy)quinazolin-7-yl)-2-amino-7-fluorobenzo[b]thiophene-3-carbonitrile

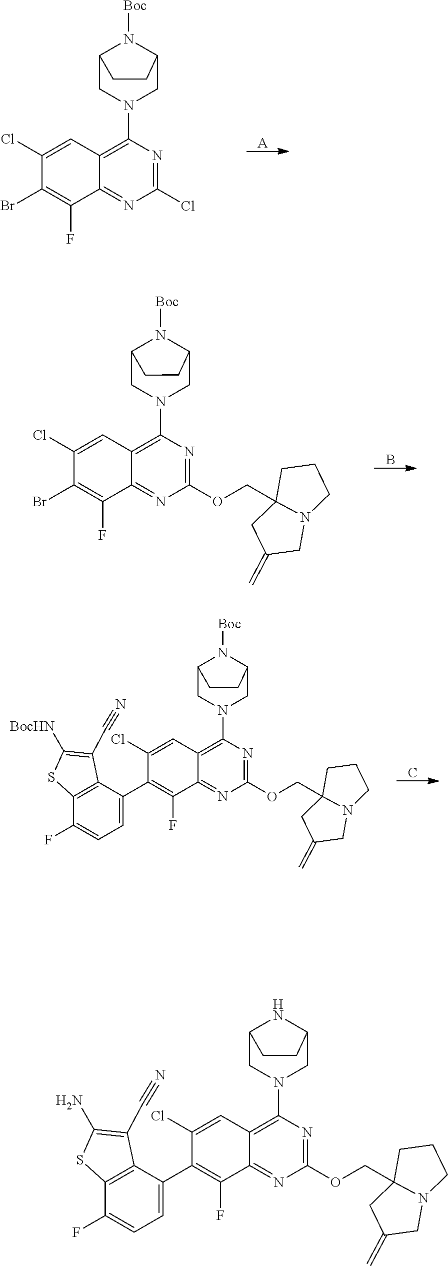

Step A. tert-butyl (1R,5S)-3-(7-bromo-6-chloro-8-fluoro-2-((2-methylenetetrahydro-1H-pyrrolizin-7a(5H)-yl)methoxy)quinazolin-4-yl)-38-diazabicyclo[3.2.1]octane-8-carboxylate. To a solution of tert-butyl 3-(7-bromo-2,6-dichloro-8-fluoro-quinazolin-4-yl)-3,8-diazabicyclo[3.2.1]octane-8-carboxylate (500 mg, 1.0 equiv) and (2-methylenetetrahydro-1H-pyrrolizin-7a(5H)-yl)methanol (227 mg, 1.5 equiv) in DMF (2.5 mL) and THE (2.5 mL) were added Cs₂CO₃ (965 mg, 3.0 equiv) and DABCO (111 mg, 1.0 equiv). The reaction was stirred at 35° C. for 1 hour. The mixture was diluted with water (10 mL) and extracted with ethyl acetate (2×10 mL). The combined organic layers were dried over anhydrous sodium sulfate, concentrated and purified by reversed phase flash chromatography [C18, 0.1% formic acid condition] to afford the title compound (280 mg, 40% yield) as yellow solid; LCMS (ESI, M+1, M+3): m/z=622.2, 624.2.

Step B. tert-butyl (1R,5S)-3-(7-(2-((tert-butoxycarbonyl)amino)-3-cyano-7-fluorobenzo[b]thiophen-4-yl)-6-chloro-8-fluoro-2-((2-methylenetetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-bromo-6-chloro-8-fluoro-2-((2-methylenetetrahydro-1H-pyrrolizin-7a(5H)-yl)methoxy)quinazolin-4-yl)-3,8-diazabicyclo[3.2.1]octane-8-carboxylate (280 mg, 1.0 equiv) and tert-butyl N-[3-cyano-4-(5,5-dimethyl-1,3,2-dioxaborinan-2-yl)-7-fluoro-benzothiophen-2-yl]carbamate (363 mg, 2.0 equiv) in toluene (3 mL) were added Cs₂CO₃ (439 mg, 3.0 equiv) and Pd(DPEphos)Cl₂ (61.8 mg, 0.2 equiv). The reaction was degassed and purged with nitrogen 3 times. The reaction was stirred at 110° C. for 1.5 hours. The mixture was quenched with water (10 mL) and extracted with EtOAc (2×10 mL). The organic layers were dried over anhydrous Na₂SO₄, concentrated and purified by reversed phase flash chromatography [C18, 0.1% formic acid condition] to afford the title compound (159 mg, 32% yield) as red solid; LCMS (ESI, M+1): m/z=834.2.

Step C. 4-(4-((1R,5S)-3,8-diazabicyclo[3.2.1]octan-3-yl)-6-chloro-8-fluoro-2-((2-methylenetetrahydro-1H-pyrrolizin-7a(5H)-yl)methoxy)quinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-6-chloro-8-fluoro-2-((2-methylenetetrahydro-1H-pyrrolizin-7a(5H)-yl)methoxy)quinazolin-4-yl)-3,8-diazabicyclo[3.2.1]octane-8-carboxylate (130 mg, 1.0 equiv) in DCM (1.5 mL) was added TFA (2.30 g, 130 equiv). The reaction was stirred at 0° C. for 1 hour. The mixture was concentrated. The residue was diluted with water (1 mL), neutralized with solid NaHCO₃ and extracted with EtOAc (2×2 mL). The combined organic layers were dried over anhydrous sodium sulfate, concentrated and purified by prep-HPLC [Phenomenex luna C18 150×25 mm×10 μm; A: water(FA); B: ACN; B %: 5%-35% over 10 min] to afford the title compound (25.3 mg, 25% yield, 0.79 HCOOH) as off-white solid; ¹H NMR (400 MHz, METHANOL-d₄) δ=7.95 (s, 1H), 7.24 (dd, J=5.2, 8.4 Hz, 1H), 7.15-6.99 (m, 1H), 5.32-5.15 (m, 2H), 4.74-4.58 (m, 4H), 4.36-4.19 (m, 1H), 3.95-3.88 (m, 2H), 3.87-3.72 (m, 3H), 3.72-3.63 (m, 1H), 3.25-3.12 (m, 1H), 3.09-2.97 (m, 1H), 2.76 (br d, J=15.6 Hz, 1H), 2.42-2.29 (m, 1H), 2.28-2.04 (m, 3H), 2.03-1.90 (m, 4H); LCMS (ESI, M+1): m/z=634.2.

### Example 135

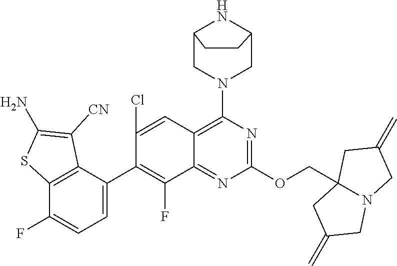 [SMILES: C=C1CN2CC(=C)CC2(COc2nc(N3CC4CCC(C3)N4)c3cc(Cl)c(-c4ccc(F)c5sc(N)c(C#N)c45)c(F)c3n2)C1]

### 4-(4-((1R,5S)-3,8-diazabicyclo[3.2.1]octan-3-yl)-6-chloro-2-((2,6-dimethylenetetrahydro-1H-pyrrolizin-7a(5H)-yl)methoxy)-8-fluoroquinazolin-7-yl)-2-amino-7-fluorobenzo[b]thiophene-3-carbonitrile

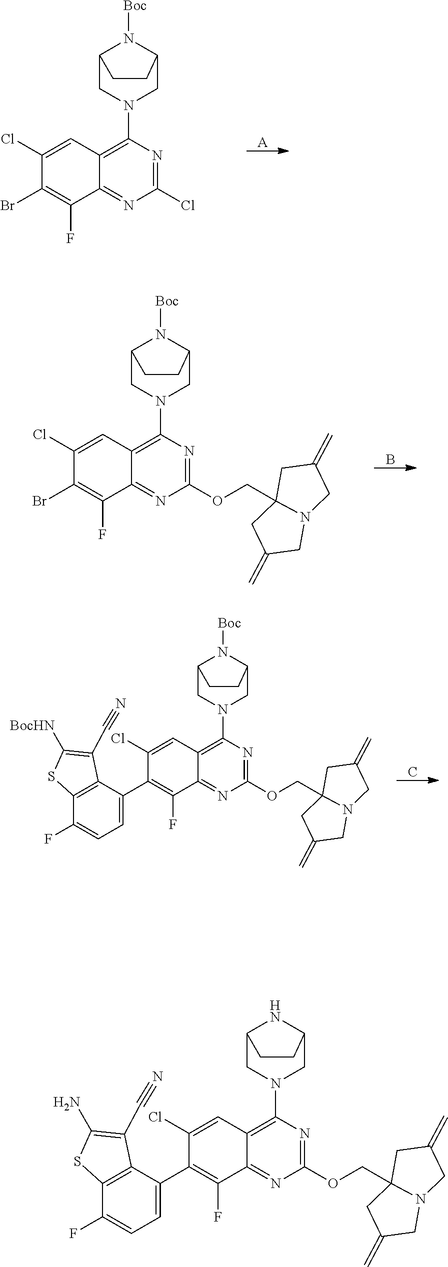

Step A. tert-butyl (1R,5S)-3-(7-bromo-6-chloro-2-((2,6-dimethylenetetrahydro-1H-pyrrolizin-7a(5H)-yl)methoxy)-8-fluoroquinazolin-4-yl)-3,8-diazabicyclo[3.2.1]octane-8-carboxylate: To a solution of tert-butyl (1R,5S)-3-(7-bromo-2,6-dichloro-8-fluoroquinazolin-4-yl)-3,8-diazabicyclo[3.2.1]octane-8-carboxylate (200 mg, 1.0 equiv) in DMF (0.5 mL) was added (2,6-dimethylenetetrahydro-1H-pyrrolizin-7a(5H)-yl)methanol (131 mg, 2.0 equiv). The reaction was stirred at 100° C. for 16 hours. The mixture was diluted with DMF (2 mL), purified by reversed phase flash chromatography (0.1% FA condition) and lyophilized to afford the title compound (70.0 mg, 28% yield) as yellow solid; LCMS (ESI, M+1, M+3): m/z=634.2, 636.2.

Step B. tert-butyl (1R,5S)-3-(7-(2-((tert-butoxycarbonyl)amino)-3-cyano-7-fluorobenzo[b]thiophen-4-yl)-6-chloro-2-((2,6-dimethylenetetrahydro-1H-pyrrolizin-7a(5H)-yl)methoxy)-8-fluoroquinazolin-4-yl)-3,8-diazabicyclo[3.2.1]octane-8-carboxylate: To a solution of tert-butyl (1R,5S)-3-(7-bromo-6-chloro-2-((2,6-dimethylenetetrahydro-1H-pyrrolizin-7a(5H)-yl)methoxy)-8-fluoroquinazolin-4-yl)-3,8-diazabicyclo[3.2.1]octane-8-carboxylate (70.0 mg, 1.0 equiv) and tert-butyl (3-cyano-4-(5,5-dimethyl-1,3,2-dioxaborinan-2-yl)-7-fluorobenzo[b]thiophen-2-yl)carbamate (53.5 mg, 1.2 equiv) in toluene (4 mL) were added Pd(DPEphos)Cl₂ (7.58 mg, 0.1 equiv) and Cs₂CO₃ (71.8 mg, 2.0 equiv). The reaction was degassed and purged with nitrogen for 3 times. The reaction was stirred at 110° C. for 2 hour under nitrogen atmosphere. The mixture was concentrated to afford the title compound (91.0 mg, crude) as yellow solid; LCMS (ESI, M+1): m/z=846.3.

Step C. 4-(4-((1R,5S)-3,8-diazabicyclo[3.2.1]octan-3-yl)-6-chloro-2-((2,6-dimethylenetetrahydro-1H-pyrrolizin-7a(5H)-yl)methoxy)-8-fluoroquinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-6-chloro-2-((2,6-dimethylenetetrahydro-1H-pyrrolizin-7a(5H)-yl)methoxy)-8-fluoroquinazolin-4-yl)-3,8-diazabicyclo[3.2.1]octane-8-carboxylate (25.0 mg, 1.0 equiv) in MeOH (1 mL) was added HCl.MeOH (4 M, 10 equiv). The reaction was stirred at 20° C. for 12 hours. The mixture was filtered, and purified by prep-HPLC (column: Phenomenex luna C18 150*25 mm*10 um; mobile phase: [water(FA)−ACN];gradient:12%-42% B over 9 min) to afford the title compound (15.2 mg, 72.7% yield, HCOOH) as yellow solid; ¹H NMR (400 MHz, CD₃OD) δ=7.89 (d, J=1.2 Hz, 1H), 7.21 (dd, J=5.2, 8.4 Hz, 1H), 7.04 (t, J=8.8 Hz, 1H), 5.09 (br d, J=2.4 Hz, 4H), 4.66-4.50 (m, 2H), 4.49-4.39 (m, 2H), 4.02-3.93 (m, 4H), 3.82-3.69 (m, 2H), 3.52 (br dd, J=1.6, 14.8 Hz, 2H), 2.92-2.62 (m, 4H), 2.02 (br s, 4H); LCMS (ESI, M+1): m/z=646.3.

### Example 136A

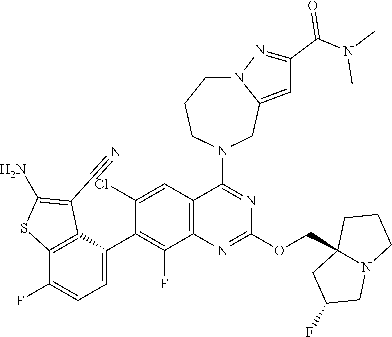

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

### Example 136B

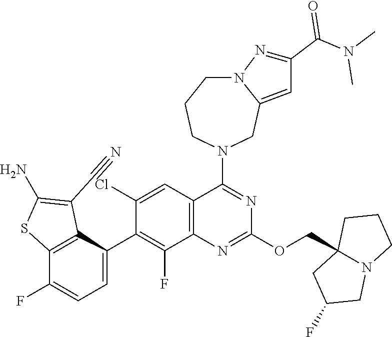

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

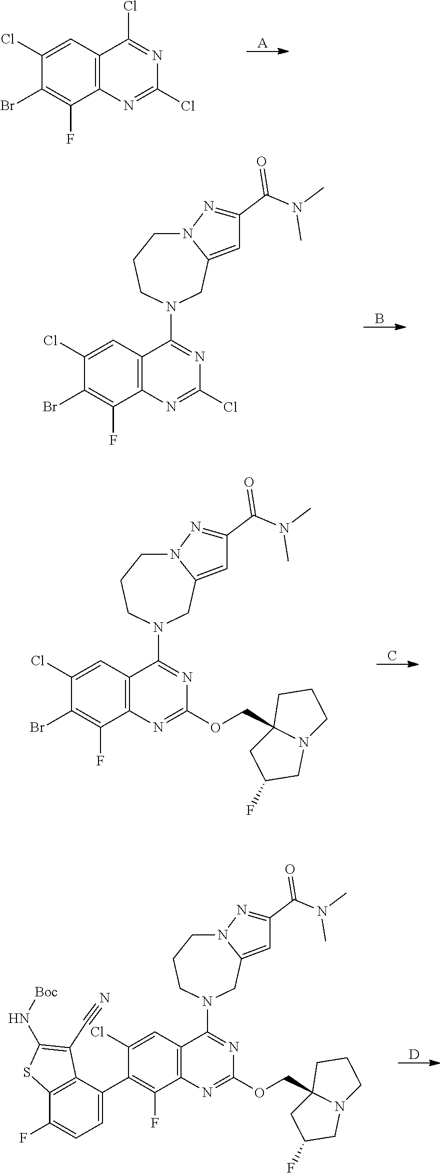

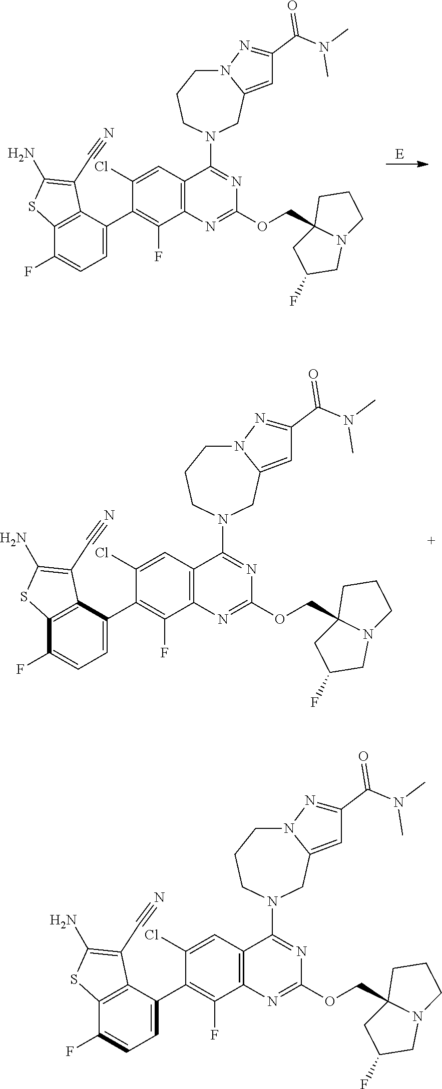

Step A. 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide: To a solution of 7-bromo-2,4,6-trichloro-8-fluoro-quinazoline (2.0 g, 1.0 equiv) and DIEA (1.96 g, 2.5 equiv) in DCM (25 mL) was added N,N-dimethyl-5,6,7,8-tetrahydro-4H-pyrazolo[1,5-a][1,4]diazepine-2-carboxamide (1.26 g, 1.0 equiv) at −40° C. The reaction was stirred at −40° C. for 4 hours. The mixture was diluted with water (40 mL) and extracted with DCM (3×60 mL). The combined organic layers were washed with brine (20 mL), dried over anhydrous sodium sulfate, concentrated and triturated with ACN (20 mL) to afford the title compound (2.85 g, 95% yield) as yellow solid; LCMS (ESI, M+1, M+3): m/z=501.1, 503.1.

Step B. 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a mixture of 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide (2.65 g, 1 equiv) and Cs₂CO₃ (5.16 g, 3.0 equiv) in DMF (25 mL) and THE (25 mL) were added ((2R,7aS)-2-fluorotetrahydro-1H-pyrrolizin-7a(5H)-yl)methanol (1.26 g, 1.5 equiv) and DABCO (651 mg, 1.1 equiv). The reaction was stirred at 20° C. for 12 hours. The mixture was diluted with water (50 mL) and extracted with ethyl acetate (5×30 mL). The organic layers were washed brine (20 mL), dried over anhydrous sodium sulfate, concentrated and purified with reversed phase flash chromatography [C18, 0.1% formic acid condition] to afford the title compound (3.3 g, 94% yield) as yellow solid; LCMS (ESI, M+1, M+3): m/z=624.1, 626.1.

Step C. tert-butyl (4-(6-chloro-4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide (1.85 g, 1.0 equiv) and tert-butyl (3-cyano-4-(5,5-dimethyl-1,3,2-dioxaborinan-2-yl)-7-fluorobenzo[b]thiophen-2-yl)carbamate (2.39 g, 2.0 equiv) in toluene (30 mL) were added Cs₂CO₃ (2.90 g, 3.0 equiv) and PdCl₂(DPEPhos) (105 mg, 0.1 equiv). The reaction was degassed and purged with nitrogen 3 times. The reaction was stirred at 100° C. for 12 hours. The mixture was diluted with H₂O (30 mL) and extracted with EtOAc (3×30 mL). The combined organic layers were dried over anhydrous Na₂SO₄, concentrated, and purified with reversed phase flash chromatography [C18, 0.1% formic acid condition] twice to afford the title compound (1.20 g, 50% yield) as brown solid; LCMS (ESI, M+1): m/z=836.3.

Step D. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of tert-butyl (4-(6-chloro-4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (1.10 g, 1.0 eq) in DCM (5.5 mL) was added TFA (6 mL). The mixture was stirred at 20° C. for 3 hrs. The mixture was concentrated, basified with NaHCO₃ aqueous (20 mL) and extracted with DCM (4×20 mL). The combined organic layers were dried over anhydrous Na₂SO₄, concentrated and purified with reversed phase flash chromatography [C18, 0.1% formic acid condition] to afford the title compound (750 mg, 75% yield) as yellow gum; LCMS (ESI, M+1): m/z=736.1.

Step E. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide (790 mg, 1 equiv) was purified by SFC [column: DAICEL CHIRALPAK AD (250 mm×30 mm,10 μm); mobile phase: CO₂-ACN/i-PrOH(0.1% NH₃H₂O); B %:50%, isocratic elution mode] to afford two peaks of the title compounds.

Example 136A (213 mg, 27% yield) as white solid; ¹H NMR (400 MHz, METHANOL-d₄) δ=8.05 (s, 1H), 7.21 (dd, J=5.2, 8.4 Hz, 1H), 7.06-7.02 (m, 1H), 6.74 (s, 1H) 5.45 (d, J=66.8 Hz, 1H), 5.10 (s, 2H), 4.52-4.32 (m, 6H), 3.61-3.59 (m, 2H), 3.34 (br s, 3H), 3.30-3.00 (m, 5H), 2.65-1.80 (m, 8H); LCMS (ESI, M+1): m/z=736.2; SFC: >99% de, Chiralpak AD-3 50×4.6 mm I.D., 3 μm column A: 40% (IPA+ACN) (0.05% DEA), B: CO₂, 3 mL/min, 220 nm, t_{R}: 1.148 min.

Example 136B (205 mg, 26% yield) as white solid. ¹H NMR (400 MHz, METHANOL-d₄) δ=8.05 (s, 1H), 7.21 (dd, J=5.2, 8.4 Hz, 1H), 7.07-7.02 (m, 1H), 6.74 (s, 1H) 5.45 (d, J=52.4 Hz, 1H), 5.10 (s, 2H), 4.52-4.33 (m, 6H), 3.67-3.54 (m, 2H), 3.34 (br s, 3H), 3.30-3.00 (m, 5H), 2.65-1.90 (m, 8H); LCMS (ESI, M+1): m/z=736.2; SFC: >99% de, Chiralpak AD-3 50×4.6 mm I.D., 3 μm column A: 40% (IPA+ACN) (0.05% DEA), B: CO₂, 3 mL/min, 220 nm, t_{R}: 1.991 min.

### Example 137

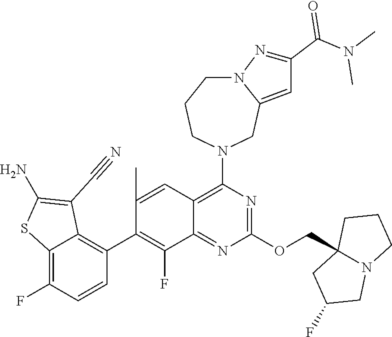 [SMILES: Cc1cc2c(N3CCCn4nc(C(=O)N(C)C)cc4C3)nc(OC[C@@]34CCCN3C[C@H](F)C4)nc2c(F)c1-c1ccc(F)c2sc(N)c(C#N)c12]

### 5-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl)-N,N-dimethyl-5,6,7,8-tetrahydro-4H-pyrazolo[1,5-a][1,4]diazepine-2-carboxamide

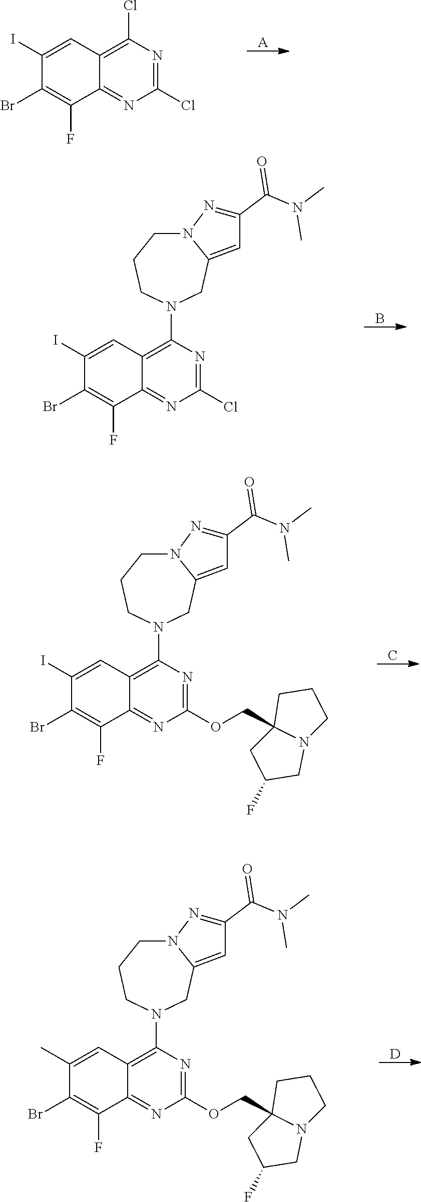

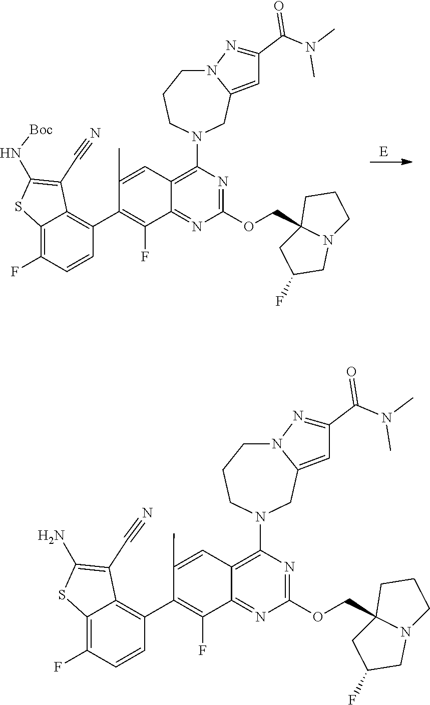 [SMILES: Cc1cc2c(N3CCCn4nc(C(=O)N(C)C)cc4C3)nc(OC[C@@]34CCCN3C[C@H](F)C4)nc2c(F)c1-c1ccc(F)c2sc(NC(=O)OC(C)(C)C)c(C#N)c12 ; Cc1cc2c(N3CCCn4nc(C(=O)N(C)C)cc4C3)nc(OC[C@@]34CCCN3C[C@H](F)C4)nc2c(F)c1-c1ccc(F)c2sc(N)c(C#N)c12]

Step A. 5-(7-bromo-2-chloro-8-fluoro-6-iodociuinazolin-4-yl)-N,N-dimethyl-5,6,7,8-tetrahydro-4H-pyrazolo[1,5-a][1,4]diazepine-2-carboxamide: To a solution of 7-bromo-2,4-dichloro-8-fluoro-6-iodo-quinazoline (1.80 g, 1.0 equiv) and DIPEA (1.38 g, 2.5 equiv) in DCM (40 mL) was added N,N-dimethyl-5,6,7,8-tetrahydro-4H-pyrazolo[1,5-a][1,4]diazepine-2-carboxamide (844 mg, 0.95 equiv) at −40° C. The reaction was stirred at −40° C. for 1 hour. The mixture was diluted with water (20 mL) and extracted with DCM (3×40 mL). The combined organic layers were washed with brine (50 mL), dried over anhydrous sodium sulfate, concentrated and triturated with acetonitrile (30 mL) to afford the title compound (1.70 g, 55% yield) as yellow solid; LCMS (ESI, M+1, M+3): m/z=593.0, 595.0.

Step B. 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide: To a mixture of 5-(7-bromo-2-chloro-8-fluoro-6-iodo-quinazolin-4-yl)-N,N-dimethyl-4,6,7,8-tetrahydropyrazolo[1,5-a][1,4]diazepine-2-carboxamide (1.60 g, 1.0 equiv) and ((2R,7aS)-2-fluorotetrahydro-1H-pyrrolizin-7a(5H)-yl)methanol (644 mg, 1.5 equiv) in THE (10 mL) and DMF (10 mL) were added Cs₂CO₃ (2.63 g, 3.0 equiv) and DABCO (302 mg, 1.0 equiv). The reaction was stirred at 20° C. for 3 hours. The mixture was diluted with water (30 mL) and extracted with EtOAc (4×50 mL). The combined organic layers were washed with brine (50 mL), dried over anhydrous sodium sulfate, concentrated and purified with reversed phase chromatography [water (0.1% formic acid)/acetonitrile=3/2] to afford the title compound (1.80 g, 92% yield) as yellow solid; LCMS (ESI, M+1, M+3): m/z=716.0, 718.0.

Step C. 5-(7-bromo-8-fluoro-2-(((2R,7aS)-2-fluorotetrahydro-1H-pyrrolizin-7a(5H)-yl)methoxy)-6-methylquinazolin-4-yl)-N,N-dimethyl-5,6,7,8-tetrahydro-4H-pyrazolo[1,5-a][1,4]diazepine-2-carboxamide: To a solution of 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide (700 mg, 1.0 equiv) and 2,4,6-trimethyl-1,3,5,2,4,6-trioxatriborinane (3.5 M, 0.42 mL, 1.5 equiv) in dioxane (9 mL) were added K₂CO₃ (1.5 M, 1.95 mL, 3.0 equiv) and Pd(dppf)Cl₂ (71.5 mg, 0.1 equiv). The reaction was degassed and purged with nitrogen 3 times. The reaction was stirred at 50° C. for 12 hours. The mixture was diluted with water (30 mL) and extracted with EtOAc (3×50 mL). The combined organic layers were washed with brine (50 mL), dried over anhydrous sodium sulfate, concentrated and purified with reversed phase chromatography [water (0.1% formic acid)/acetonitrile=3/2] to afford the title compound (320 mg, 47% yield) as brown solid; LCMS (ESI, M+1): m/z=604.2.

Step D. tert-butyl (3-cyano-4-(4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate: To a mixture of 5-(7-bromo-8-fluoro-2-(((2R,7aS)-2-fluorotetrahydro-1H-pyrrolizin-7a(5H)-yl)methoxy)-6-methylquinazolin-4-yl)-N,N-dimethyl-5,6,7,8-tetrahydro-4H-pyrazolo[1,5-a][1,4]diazepine-2-carboxamide (290 mg, 1.0 equiv) and Cs₂CO₃ (469 mg, 3.0 equiv) in toluene (5.0 mL) were added tert-butyl (3-cyano-4-(5,5-dimethyl-1,3,2-dioxaborinan-2-yl)-7-fluorobenzo[b]thiophen-2-yl)carbamate (291 mg, 1.5 equiv) and Pd(DPEphos)Cl₂ (33.0 mg, 0.1 equiv). The reaction was degassed and purged with nitrogen 3 times. The reaction was stirred at 105° C. for 4 hours. The mixture was diluted with water (10 mL) and extracted with EtOAc (4×10 mL). The combined organic layers were washed with brine (50 mL), dried over anhydrous sodium sulfate, concentrated and purified with reversed phase chromatography [water (0.1% formic acid)/acetonitrile=1/1] to afford the title compound (187 mg, 46% yield) as yellow solid; LCMS (ESI, M+1): m/z=816.3.

Step E. 5-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl)-N,N-dimethyl-5,6,7,8-tetrahydro-4H-pyrazolo[1,5-a][1,4]diazepine-2-carboxamide: To a solution of tert-butyl (3-cyano-4-(4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate (95.0 mg, 1.0 equiv) in DCM (1.0 mL) was added TFA (2.0 mL) at 0° C. The reaction was stirred at 20° C. for 1 hour. The mixture was quenched with saturated NaHCO₃ aqueous (10 mL) at 0° C. and extracted with DCM (4×10 mL). The combined organic layers were washed with brine (20 mL), dried over anhydrous sodium sulfate, concentrated and purified with prep-HPLC [column: Phenomenex C18 150×25 mm×10 μm; mobile phase: water (formic acid)-acetonitrile; gradient:16%-46% over 10 minutes] and lyophilized to afford the title compound (42.7 mg, 49% yield, HCOOH salt) as white solid; ¹H NMR (400 MHz, METHANOL-d4) δ=8.53 (s, 1H), 7.78 (s, 1H), 7.16 (dd, J=5.2, 8.4 Hz, 1H), 7.08-7.00 (m, 1H), 6.73 (s, 1H), 5.52-5.29 (m, 1H), 5.10 (s, 2H), 4.56-4.47 (m, 2H), 4.47-4.28 (m, 4H), 3.72-3.42 (m, 3H), 3.35 (s, 3H), 3.27-3.15 (m, 1H), 3.08 (s, 3H), 2.55-2.22 (m, 5H), 2.22-2.07 (m, 5H), 2.06-1.94 (m, 1H) LCMS (ESI, M+1): m/z=716.4.

### Example 138

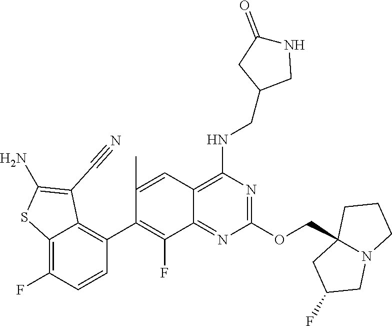 [SMILES: Cc1cc2c(NCC3CNC(=O)C3)nc(OC[C@@]34CCCN3C[C@H](F)C4)nc2c(F)c1-c1ccc(F)c2sc(N)c(C#N)c12]

### 2-amino-7-fluoro-4-(8-fluoro-2-(((2R,7aS)-2-fluorotetrahydro-1H-pyrrolizin-7a(5H)-yl)methoxy)-6-methyl-4-(((5-oxopyrrolidin-3-yl)methyl)amino)quinazolin-7-yl)benzo[b]thiophene-3-carbonitrile

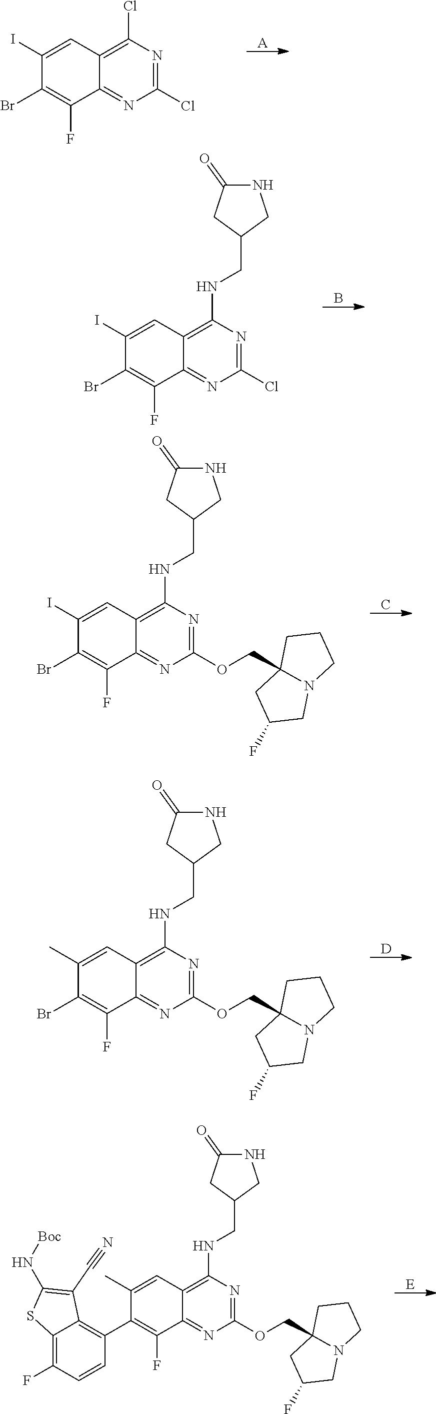

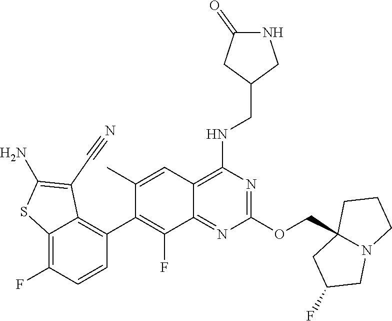 [SMILES: Cc1cc2c(NCC3CNC(=O)C3)nc(OC[C@@]34CCCN3C[C@H](F)C4)nc2c(F)c1-c1ccc(F)c2sc(N)c(C#N)c12]

Step A. 4-(7-bromo-2-chloro-8-fluoro-6-iodoquinazolin-4-yl)amino)methyl)pyrrolidin-2-one: To a solution of 7-bromo-2,4-dichloro-8-fluoro-6-iodoquinazoline (1.80 g, 1.0 equiv) and DIEA (2.48 g, 4.5 equiv) in DCM (50 mL) was added 4-(aminomethyl)pyrrolidin-2-one hydrogen chloride (610 mg, 0.9 equiv) at −40° C. The reaction was stirred at −40° C. for 1 hour. The mixture was quenched with H₂O (50 mL) at −40° C. and extracted with DCM (3×40 mL). The combined organic layers were washed with brine (50 mL), dried over anhydrous sodium sulfate, concentrated and triturated with acetonitrile (30 mL) to afford the title compound (1.64 g, 498 yield) as yellow solid; LCMS (ESI, M+1, M+3): m/z=498.9, 500.9.

Step B. 4-(((7-bromo-8-fluoro-2-(((2R,7aS)-2-fluorotetrahydro-1H-pyrrolizin-7a(5H)-yl)methoxy)-6-iodoquinazolin-4-yl)amino)methyl)pyrrolidin-2-one: To a solution of 4-(((7-bromo-2-chloro-8-fluoro-6-iodoquinazolin-4-yl)amino)methyl)pyrrolidin-2-one (870 mg, 1.0 equiv) and DIPEA (450 mg, 2.0 equiv) in DMSO (0.6 mL) was added ((2R,7aS)-2-fluorotetrahydro-1H-pyrrolizin-7a(5H)-yl)methanol (2.22 g, 8.0 equiv). The reaction was stirred at 100° C. for 14 hours. The mixture purified with reversed phase chromatography [water (0.1% formic acid)/acetonitrile=2/1] to afford the title compound (1.0 g, 81% yield) as yellow solid; LCMS (ESI, M+1, M+3): m/z=622.0, 624.0.

Step C. 4-(((7-bromo-8-fluoro-2-(((2R,7aS)-2-fluorotetrahydro-1H-pyrrolizin-7a(5H)-yl)methoxy)-6-methylquinazolin-4-yl)amino)methyl)pyrrolidin-2-one: To a mixture of 4-(((7-bromo-8-fluoro-2-(((2R,7aS)-2-fluorotetrahydro-1H-pyrrolizin-7a(5H)-yl)methoxy)-6-iodoquinazolin-4-yl)amino)methyl)pyrrolidin-2-one (310 mg, 1.0 equiv) and K₂CO₃ (207 mg, 3.0 equiv) in DMSO (3 mL) were added 2,4,6-trimethyl-1,3,5,2,4,6-trioxatriborinane (3.5 M, 285 μL, 2.0 equiv) and Pd(dppf)Cl₂ (36.4 mg, 0.1 equiv). The reaction was degassed and purged with nitrogen 3 times. The reaction was stirred at 55° C. for 20 hours. The mixture was diluted with water (10 mL) and extracted with EtOAc (4×10 mL). The combined organic layers were washed with brine (20 mL), dried over anhydrous sodium sulfate, concentrated and purified with reversed phase chromatography [water (0.1% formic acid)/acetonitrile=2/1] to afford the title compound (107 mg, 34% yield) as yellow solid; LCMS (ESI, M+1, M+3): m/z=510.1, 512.1.

Step D. tert-butyl (3-cyano-7-fluoro-4-(8-fluoro-2-(((2R,7aS)-2-fluorotetrahydro-1H-pyrrolizin-7a(5H)-yl)methoxy)-6-methyl-4-(((5-oxopyrrolidin-3-yl)methyl)amino)quinazolin-7-yl)benzo[b]thiophen-2-yl)carbamate: To a mixture of 4-(((7-bromo-8-fluoro-2-(((2R,7aS)-2-fluorotetrahydro-1H-pyrrolizin-7a(5H)-yl)methoxy)-6-methylquinazolin-4-yl)amino)methyl)pyrrolidin-2-one (40 mg, 1.0 equiv) and tert-butyl (3-cyano-4-(5,5-dimethyl-1,3,2-dioxaborinan-2-yl)-7-fluorobenzo[b]thiophen-2-yl)carbamate (63.4 mg, 2.0 equiv) in dioxane (0.8 mL) were added Cs₂CO₃ (63.8 mg, 2.5 equiv) and PdCl₂(DPEPhos) (5.39 mg, 0.1 equiv). The reaction was degassed and purged with nitrogen 3 times. The reaction was stirred at 95° C. for 3 hours. The mixture was diluted with water (10 mL) and extracted with EtOAc (2×10 mL). The combined organic layers were washed with brine (10 mL), dried over anhydrous sodium sulfate, concentrated and purified with reversed phase chromatography [water (0.1% formic acid)/acetonitrile=3/2] to afford the title compound (25 mg, 44%) as white solid; LCMS (ESI, M+1): m/z=722.2.

Step E. 2-amino-7-fluoro-4-(8-fluoro-2-(((2R,7aS)-2-fluorotetrahydro-1H-pyrrolizin-7a(5H)-yl)methoxy)-6-methyl-4-(((5-oxopyrrolidin-3-yl)methyl)amino)quinazolin-7-yl)benzo[b]thiophene-3-carbonitrile: To a solution of tert-butyl (3-cyano-7-fluoro-4-(8-fluoro-2-(((2R,7aS)-2-fluorotetrahydro-1H-pyrrolizin-7a(5H)-yl)methoxy)-6-methyl-4-(((5-oxopyrrolidin-3-yl)methyl)amino)quinazolin-7-yl)benzo[b]thiophen-2-yl)carbamate (40.0 mg, 1.0 equiv) in DCM (0.5 mL) was added TFA (1 mL) at 0° C. The reaction was stirred at 20° C. for 1 hour. The mixture was poured into saturated NaHCO₃ aqueous solution (5 mL) and DCM (5 mL) at 0° C., and extracted with DCM (2×5 mL). The combined organic layers were washed with brine (5 mL), dried over anhydrous sodium sulfate, concentrated and purified with prep-HPLC [column: Phenomenex C18 150×25 mm×10 μm; mobile phase: water (formic acid)-acetonitrile; gradient:19%-49% over 10 minutes] to afford the title compound (27.0 mg, 78% yield, HCOOH salt) as off-white solid; ¹H NMR (400 MHz, CD₃OD) δ=7.84 (s, 1H), 7.17 (ddd, J=1.6, 5.2, 8.4 Hz, 1H), 7.08-7.02 (m, 1H), 5.58-5.37 (m, 1H), 4.68-4.45 (m, 2H), 3.94-3.63 (m, 5H), 3.62-3.54 (m, 1H), 3.41-3.33 (m, 1H), 3.27 (dd, J=4.8, 10.4 Hz, 1H), 3.05-2.95 (m, 1H), 2.66-2.46 (m, 3H), 2.45-2.30 (m, 1H), 2.25 (ddd, J=3.6, 5.2, 16.8 Hz, 3H), 2.18 (s, 3H), 2.14-2.00 (m, 1H); LCMS (ESI, M+1): m/z=622.3.

### Example 139

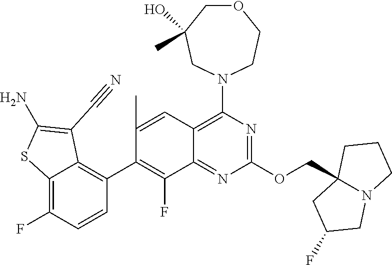 [SMILES: Cc1cc2c(N3CCOC[C@@](C)(O)C3)nc(OC[C@@]34CCCN3C[C@H](F)C4)nc2c(F)c1-c1ccc(F)c2sc(N)c(C#N)c12]

### 2-amino-7-fluoro-4-(8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)-6-methylquinazolin-7-yl)benzo[b]thiophene-3-carbonitrile

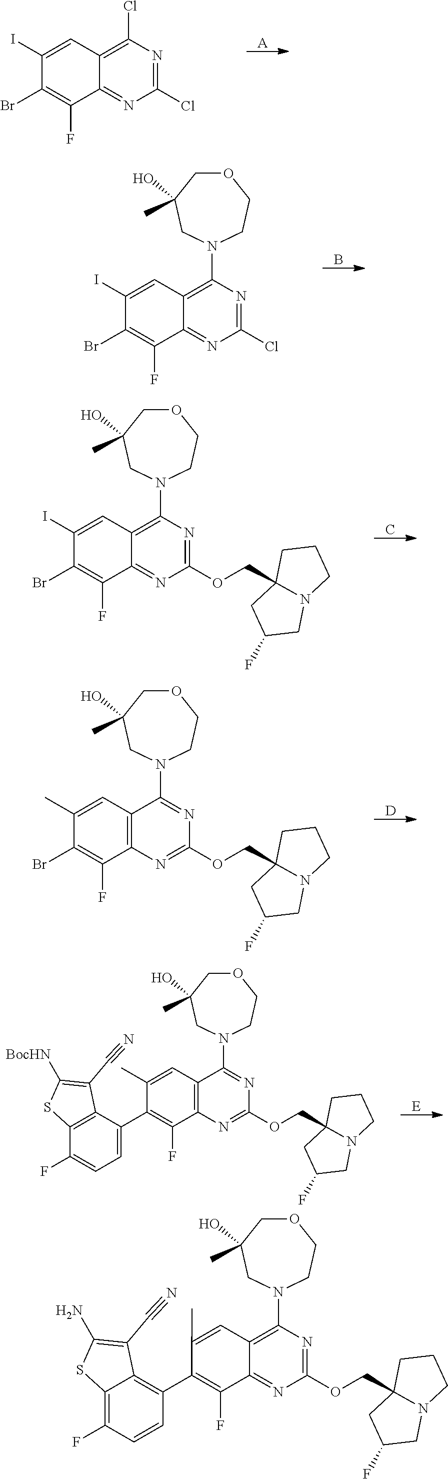

Step A. (S)-4-(7-bromo-2-chloro-8-fluoro-6-iodoquinazolin-4-yl)-6-methyl-1,4-oxazepan-6-ol: To a solution of 7-bromo-2,4-dichloro-8-fluoro-6-iodoquinazoline (1.50 g, 1.0 equiv) and DIEA (1.38 g, 3.0 equiv) in DCM (10 mL) was added (S)-6-methyl-1,4-oxazepan-6-ol (466 mg, 1.0 equiv) at 0° C. The reaction was stirred at 0° C. for 1 hour. The mixture was quenched with H₂O (20 mL) and extracted with DCM (2×15 mL). The combined organic layers were washed with brine (10 mL), dried over anhydrous sodium sulfate, concentrated, and triturated with PE:EA=3:1, (30 mL) at 20° C. for 15 minutes to afford the title compound (1.50 g, 76% yield) as yellow solid; LCMS (ESI, M+1, M+3): m/z=515.9, 517.9.

Step B. (S)-4-(7-bromo-8-fluoro-2-(((2R,7aS)-2-fluorotetrahydro-1H-pyrrolizin-7a(5H)-yl)methoxy)-6-iodoquinazolin-4-yl)-6-methyl-1,4-oxazepan-6-ol: To a mixture of (S)-4-(7-bromo-2-chloro-8-fluoro-6-iodoquinazolin-4-yl)-6-methyl-1,4-oxazepan-6-ol (1.30 g, 1.0 equiv), ((2R,7aS)-2-fluorotetrahydro-1H-pyrrolizin-7a(5H)-yl)methanol (601 mg, 1.5 equiv) and Cs₂CO₃ (2.40 g, 3.0 equiv) in THE (15 mL) and DMF(15 ml) was added DABCO (282 mg, 1.0 equiv). The reaction was stirred at 25° C. for 12 hours. The mixture was quenched with H₂O (50 mL) and extracted with EtOAc (3×50 mL). The combined organic layers were washed with brine (50 mL), dried over anhydrous sodium sulfate, and concentrated to afford the title compound (3.80 g, crude) as yellow oil.

Step C. (S)-4-(7-bromo-8-fluoro-2-(((2R,7aS)-2-fluorotetrahydro-1H-pyrrolizin-7a(5H)-yl)methoxy)-6-methylquinazolin-4-yl)-6-methyl-1,4-oxazepan-6-ol: To a mixture of (S)-4-(7-bromo-8-fluoro-2-(((2R,7aS)-2-fluorotetrahydro-1H-pyrrolizin-7a(5H)-yl)methoxy)-6-iodoquinazolin-4-yl)-6-methyl-1,4-oxazepan-6-ol (1.00 g, 1.0 equiv), 2,4,6-trimethyl-1,3,5,2,4,6-trioxatriborinane (510 mg, 1.3 equiv) and aqueous K₂CO₃ (1 mL, 1.5M) in dioxane (8 mL) was added Pd(dppf)Cl₂ (114 mg, 0.1 equiv). The reaction was degassed and purged with nitrogen 3 times. The reaction was stirred at 50° C. for 12 hours. The mixture was quenched with H₂O (10 mL) and extracted with EtOAc (3×10 mL). The combined organic layers were washed with brine (20 mL), dried over anhydrous sodium sulfate, concentrated and purified by reversed phase flash chromatography [C18, 0.1% formic acid condition] to afford the title compound (220 mg, 27% yield) as yellow oil; LCMS (ESI, M+1, M+3): m/z=527.2, 529.2.

Step D. tert-butyl (3-cyano-7-fluoro-4-(8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)-6-methylquinazolin-7-yl)benzo[b]thiophen-2-yl)carbamate: To a mixture of (S)-4-(7-bromo-8-fluoro-2-(((2R,7aS)-2-fluorotetrahydro-1H-pyrrolizin-7a(5H)-yl)methoxy)-6-methylquinazolin-4-yl)-6-methyl-1,4-oxazepan-6-ol (200 mg, 1.0 equiv), tert-butyl (3-cyano-4-(5,5-dimethyl-1,3,2-dioxaborinan-2-yl)-7-fluorobenzo[b]thiophen-2-yl)carbamate (230 mg, 1.5 equiv) and Cs₂CO₃ (370 mg, 1.5 equiv) in toluene (2.0 mL) was added Pd(DPEphos)Cl₂ (26.1 mg, 0.1 equiv). The reaction was degassed and purged with nitrogen 3 times. The reaction was stirred at 100° C. for 3 hours. The mixture was diluted with H₂O (10 mL) and extracted with EtOAc (3×10 mL). The combined organic layers were washed with brine (20 mL), dried over anhydrous sodium sulfate, concentrated, and purified by reversed phase flash chromatography [C18, 0.1% formic acid condition] to afford the title compound (75.0 mg, 25% yield) as yellow solid; LCMS (ESI, M+1): m/z=739.3.

Step E. 2-amino-7-fluoro-4-(8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)-6-methylquinazolin-7-

yl)benzo[b]thiophene-3-carbonitrile: To a solution of tert-butyl (3-cyano-7-fluoro-4-(8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)-6-methylquinazolin-7-yl)benzo[b]thiophen-2-yl)carbamate (70.0 mg, 1.0 equiv) in DCM (0.5 mL) was added TFA (1.54 g, 114 equiv) at 0° C. The reaction was stirred at 25° C. for 0.5 hours. The mixture was quenched with saturated NaHCO₃ aqueous (10 mL) and extracted with EtOAc (2×10 mL). The combined organic layers were washed with brine (10 mL), dried over anhydrous sodium sulfate, concentrated and purified by Prep-HPLC [column: Phenomenex luna C18 150×25 mm×10 μm; A: water (FA), B: ACN, B %: 16%-46% over 10 min] to afford the title compound (36.8 mg, 59% yield, HCOOH salt) as pink solid; ¹H NMR (400 MHz, METHANOL-d₄) δ=8.02 (d, J=4.4 Hz, 1H), 7.17 (dd, J=5.2, 8.4 Hz, 1H), 7.08-7.00 (m, 1H), 5.52-5.31 (m, 1H), 4.55-4.26 (m, 4H), 4.06-3.98 (m, 2H), 3.96-3.84 (m, 2H), 3.72-3.65 (m, 1H), 3.65-3.43 (m, 4H), 3.23 (br d, J=2.4 Hz, 1H), 2.57-2.35 (m, 2H), 2.35-2.22 (m, 1H), 2.21-2.08 (m, 5H), 2.07-1.92 (m, 1H), 1.26 (s, 3H); LCMS (ESI, M+1): m/z=639.2.

### Example 140

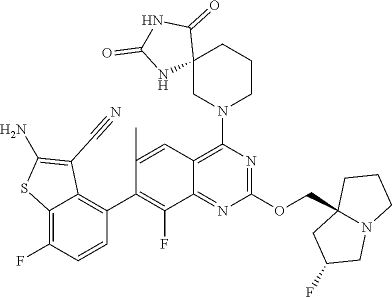 [SMILES: Cc1cc2c(N3CCC[C@]4(C3)NC(=O)NC4=O)nc(OC[C@@]34CCCN3C[C@H](F)C4)nc2c(F)c1-c1ccc(F)c2sc(N)c(C#N)c12]

### 2-amino-4-(4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile

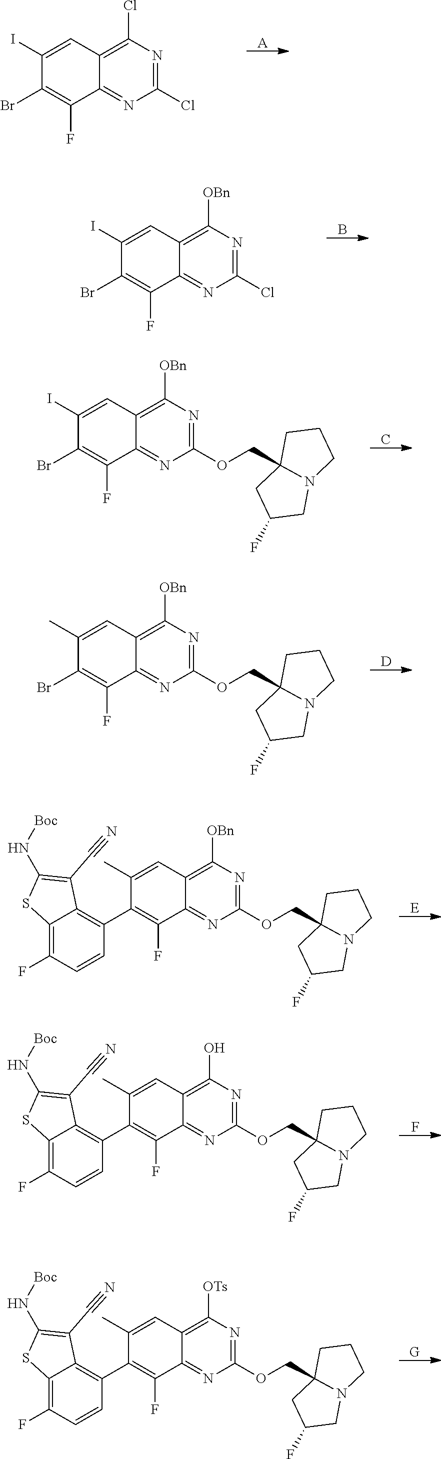

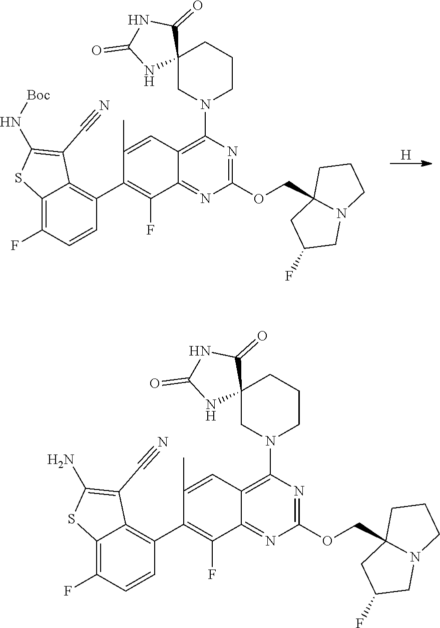

Step A. 4-(benzyloxy)-7-bromo-2-chloro-8-fluoro-6-iodoquinazoline: To a solution of 7-bromo-2,4-dichloro-8-fluoro-6-iodoquinazoline (2.0 g, 1.0 equiv) and BnOH (564 mg, 1.1 equiv) in THE (20 mL) was added t-BuOK (1 M, 5.69 mL, 1.2 equiv) dropwise at −40° C. under N₂ atmosphere. The reaction was stirred at 20° C. for 1 hour. The mixture was diluted with water (80 mL) and extracted with EtOAc (2×40 mL). The combined organic layers were washed with brine (40 mL), dried over anhydrous sodium sulfate, and concentrated to afford the title compound (2.0 g, crude) as white solid; LCMS (ESI, M+1, M+3): m/z=492.9, 494.9.

Step B. 4-(benzyloxy)-7-bromo-8-fluoro-2-(((2R,7aS)-2-fluorotetrahydro-1H-pyrrolizin-7a(5H)-yl)methoxy)-6-iodoquinazoline: To a mixture of 4-(benzyloxy)-7-bromo-2-chloro-8-fluoro-6-iodoquinazoline (1.7 g, 1.0 equiv) and DIEA (13.4 g, 30.0 equiv) was added ((2R,7aS)-2-fluorotetrahydro-1H-pyrrolizin-7a(5H)-yl)methanol (1.1 g, 2.0 equiv). The reaction was stirred at 90° C. for 4 hours. The mixture was diluted with water (100 mL) and extracted with EtOAc (6×40 mL). The combined organic layers were washed with brine (100 mL), dried over anhydrous sodium sulfate, concentrated, and purified with reversed phase chromatography [0.1% FA condition] to afford the title compound (700 mg, 30% yield) as yellow solid; LCMS (ESI, M+1, M+3): m/z=616.0, 618.0.

Step C. 4-(benzyloxy)-7-bromo-8-fluoro-2-(((2R,7aS)-2-fluorotetrahydro-1H-pyrrolizin-7a(5H)-yl)methoxy)-6-methylquinazoline: To a solution of 4-(benzyloxy)-7-bromo-8-fluoro-2-(((2R,7aS)-2-fluorotetrahydro-1H-pyrrolizin-7a(5H)-yl)methoxy)-6-iodoquinazoline (500 mg, 1.0 equiv) and 2,4,6-trimethyl-1,3,5,2,4,6-trioxatriborinane (3.5 M, 348 μL, 1.5 equiv) in dioxane (6.0 mL) were added K₂CO₃ (1.5 M in H₂O, 1.62 mL, 3.0 equiv) and Pd(dppf)Cl₂ (59 mg, 0.1 equiv). The reaction was degassed and purged with N₂ for 3 times. The reaction was stirred at 50° C. for 12 hours. The mixture was diluted with water (50 mL) and extracted with EtOAc (3×25 mL). The combined organic layers were washed with brine (25 mL), dried over anhydrous sodium sulfate, concentrated and purified with reversed phase chromatography [0.1% FA condition] to afford the title compound (280 mg, 31% yield) as brown solid; LCMS (ESI, M+1, M+3): m/z=504.2, 506.2.

Step D. tert-butyl (4-(4-(benzyloxy)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)carbamate: To a mixture of 4-(benzyloxy)-7-bromo-8-fluoro-2-(((2R,7aS)-2-fluorotetrahydro-1H-pyrrolizin-7a(5H)-yl)methoxy)-6-methylquinazoline (280 mg, 1.0 equiv) and tert-butyl (3-cyano-4-(5,5-dimethyl-1,3,2-dioxaborinan-2-yl)-7-fluorobenzo[b]thiophen-2-yl)carbamate (359 mg, 2.0 equiv) in toluene (6 mL) were added Cs₂CO₃ (434 mg, 3.0 equiv) and Pd(DPEphos)Cl₂ (31.7 mg, 0.1 equiv). The reaction was degassed and purged with N₂ for 3 times. The reaction was stirred at 110° C. for 2 hours. The mixture was diluted with water (10 mL) and extracted with EtOAc (5×5 mL). The organic layers were washed with brine (10 mL), dried over anhydrous sodium sulfate, concentrated, and purified with reversed phase chromatography [0.1% FA condition] to afford the title compound (130 mg, 33% yield) as brown solid; LCMS (ESI, M+1): m/z=716.3.

Step E. tert-butyl (3-cyano-7-fluoro-4-(8-fluoro-2-(((2R,7aS)-2-fluorotetrahydro-1H-pyrrolizin-7a(5H)-yl)methoxy)-4-hydroxy-6-methylquinazolin-7-yl)benzo[b]thiophen-2-yl)carbamate: To a solution of tert-butyl (4-(4-(benzyloxy)-8-fluoro-2-(((2R,7aS)-2-fluorotetrahydro-1H-pyrrolizin-7a(5H)-yl)methoxy)-6-methylquinazolin-7-yl)-3-cyano-7-fluorobenzo[b]thiophen-2-yl)carbamate (110 mg, 1.0 equiv) in methanol (2.0 mL) was added Pd/C (200 mg, 10% purity) under N₂ atmosphere. The reaction was degassed and purged with H₂ for 3 times. The reaction was stirred at 20° C. for 2 hours under H₂ atmosphere (15 psi). The mixture was filtered through a pad of Celite and concentrated to afford the title compound (90 mg, crude) as brown solid; LCMS (ESI, M+1): m/z=626.2.

Step F. 7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl 4-methylbenzenesulfonate: To a solution of tert-butyl (3-cyano-7-fluoro-4-(8-fluoro-2-(((2R,7aS)-2-fluorotetrahydro-1H-pyrrolizin-7a(5H)-yl)methoxy)-4-hydroxy-6-methylquinazolin-7-yl)benzo[b]thiophen-2-yl)carbamate (90.0 mg, 1.0 equiv) and DIEA (56.0 mg, 3.0 equiv) in DCM (2.0 mL) was added TsCl (41.0 mg, 1.5 equiv). The reaction was stirred at 20° C. for 48 hours. The mixture was diluted with water (10 mL) and extracted with DCM (5×5 mL). The organic layers were washed with brine (10 mL), dried over anhydrous sodium sulfate, and concentrated to afford the title compound (138 mg, crude) as brown solid; LCMS (ESI, M+1): m/z=780.2.

Step G. tert-butyl (3-cyano-4-(4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate: To a solution of 7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl 4-methylbenzenesulfonate (61.0 mg, 1.0 equiv) and DIEA (31.0 mg, 3.0 equiv) in DMF (1.0 mL) was added (R)-1,3,7-triazaspiro[4.5]decane-2,4-dione (93.0 mg, 7.0 equiv). The reaction was stirred at 40° C. for 2 hours. The mixture was filtered and purified with reversed phase chromatography [0.1% FA condition] to afford the title compound (20 mg, 31% yield) as yellow solid; LCMS (ESI, M+1): m/z=777.3.

Step H. 2-amino-4-(4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile: To a solution of tert-butyl (3-cyano-4-(4-((R)-2,4-dioxo-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate (15.0 mg, 1.0 equiv) in DCM (0.5 mL) was added TFA (1.5 mL) at 0° C. The reaction was stirred at 20° C. for 1 hour. The mixture was concentrated. The residue was diluted with EtOAc (5.0 mL) and saturated NaHCO₃ aqueous (10 mL) at 0° C. The mixture was extracted with EtOAc (4×4.0 mL). The combined organic layers washed with brine (5.0 mL), dried over anhydrous sodium sulfate, concentrated, and purified with prep-HPLC [column: Waters Xbridge 150×25 mm×5 m; mobile phase: water (ammonia hydroxide v/v)−ACN; gradient: 30%-60% over 9 minutes] to afford the title compound (4.6 mg, 35% yield) as pink solid; ¹H NMR (400 MHz, CD₃OD) δ=7.75 (d, J=5.6 Hz, 1H), 7.16 (ddd, J=2.8, 5.2, 8.4 Hz, 1H), 7.07-6.97 (m, 1H), 5.39-5.21 (m, 1H), 4.46-4.31 (m, 2H), 4.30-4.13 (m, 2H), 3.60-3.40 (m, 2H), 3.28-3.08 (m, 3H), 3.08-2.90 (m, 1H), 2.39-2.17 (m, 3H), 2.16 (s, 3H), 2.13 (br s, 1H), 2.06-1.82 (m, 6H); LCMS (ESI, M+1): m/z=677.2.

### Example 141

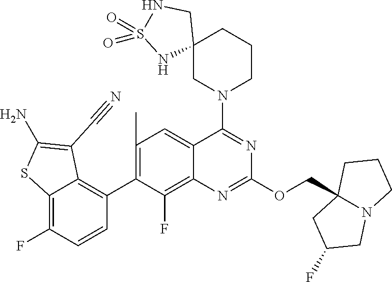 [SMILES: Cc1cc2c(N3CCC[C@]4(CNS(=O)(=O)N4)C3)nc(OC[C@@]34CCCN3C[C@H](F)C4)nc2c(F)c1-c1ccc(F)c2sc(N)c(C#N)c12]

### 2-amino-4-(4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile

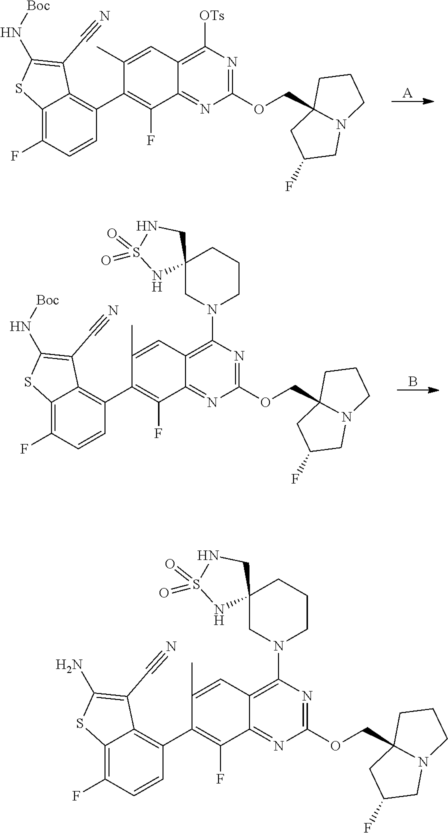

Step A. tert-butyl (3-cyano-4-(4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate: To a solution of 7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-methylquinazolin-4-yl 4-methylbenzenesulfonate (61.5 mg, 1.0 equiv) and DIEA (41.2 μL, 3.0 equiv) in DMF (0.5 mL) was added (R)-2-thia-1,3,7-triazaspiro[4.5]decane 2,2-dioxide (90.5 mg, 6.0 equiv). The reaction was stirred at 40° C. for 2 hours. The mixture was filtered and purified with reversed phase chromatography [water (0.1% FA)/acetonitrile=3/2] to afford the title compound (30.0 mg, 47% yield) as yellow solid; LCMS (ESI, M+1): m/z=799.2.

Step B. 2-amino-4-(4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophene-3-carbonitrile: To a solution of tert-butyl (3-cyano-4-(4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)-6-methylquinazolin-7-yl)-7-fluorobenzo[b]thiophen-2-yl)carbamate (25 mg, 1.0 equiv) in DCM (0.2 mL) was added TFA (0.3 mL) at 0° C. The reaction was stirred at 20° C. for 1 hour. The mixture was concentrated. The residue was diluted with EtOAc (5 mL) and saturated NaHCO₃ aqueous (5.0 mL) at 0° C. The mixture was extracted with EtOAc (2×5 mL). The combined organic layers washed with brine (5.0 mL), dried over anhydrous sodium sulfate, concentrated and purified with prep-HPLC[column: Waters Xbridge 150×25 mm×5 μm; mobile phase: water (ammonia hydroxide v/v)−ACN; gradient: 40%-70% over 9 minutes] to afford the title compound (4.34 mg, 20% yield) as white solid; ¹H NMR (400 MHz, CD₃OD) δ=7.79 (d, J=13.6 Hz, 1H), 7.18 (dt, J=5.2, 8.0 Hz, 1H), 7.09-7.00 (m, 1H), 5.40-5.21 (m, 1H), 4.31-4.24 (m, 2H), 4.17 (br dd, J=9.2, 13.2 Hz, 1H), 4.08-3.92 (m, 1H), 3.83-3.64 (m, 2H), 3.52-3.43 (m, 1H), 3.39-3.35 (m, 1H), 3.29-3.15 (m, 3H), 3.08-2.98 (m, 1H), 2.43-2.14 (m, 6H), 2.11-2.06 (m, 1H), 2.04-1.83 (m, 6H); LCMS (ESI, M+1): m/z=699.2.

### Example 142

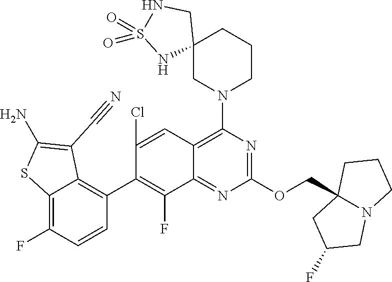 [SMILES: N#Cc1c(N)sc2c(F)ccc(-c3c(Cl)cc4c(N5CCC[C@]6(CNS(=O)(=O)N6)C5)nc(OC[C@@]56CCCN5C[C@H](F)C6)nc4c3F)c12]

### 2-amino-4-(6-chloro-4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

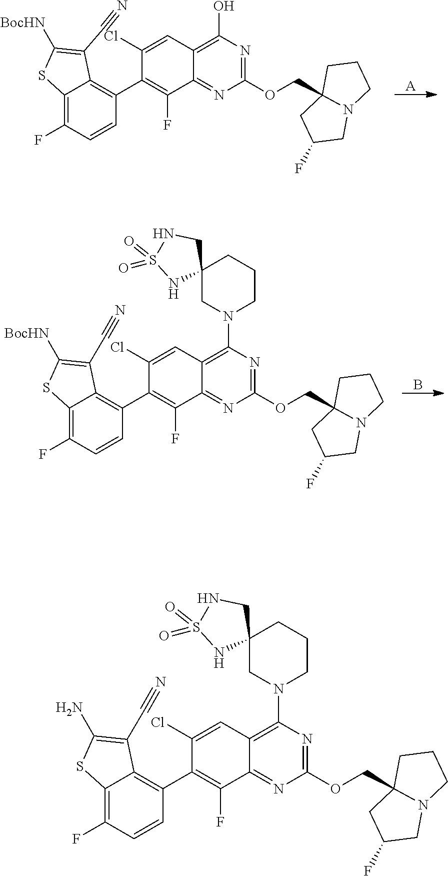

Step A. tert-butyl (4-(6-chloro-4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) and TEA (47.0 mg, 3.0 equiv) in DMSO (1.00 mL) was added PYBOP (121 mg, 1.5 equiv). The reaction was stirred at 30° C. for 0.5 hours. Then to the mixture was added (R)-2-thia-1,3,7-triazaspiro[4.5]decane 2,2-dioxide (88.8 mg, 3.0 equiv). The reaction was stirred at 30° C. for 12 hours. The mixture was diluted with water (10 mL) and extracted with EtOAc (2×10 mL). The combined organic layers were dried over anhydrous sodium sulfate, concentrated, and purified with reversed phase flash chromatography [C18, 0.1% formic acid condition] to afford the title compound (35.0 mg, 26% yield) as yellow solid; LCMS (ESI, M+1): m/z=819.3.

Step B. 2-amino-4-(6-chloro-4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-((R)-2,2-dioxido-2-thia-1,3,7-triazaspiro[4.5]decan-7-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (15.0 mg, 1.0 equiv) in DCM (0.50 mL) was added TFA (1.54 g, 735 equiv). The reaction was stirred at 25° C. for 2 hours. The mixture was concentrated, basified by saturated NaHCO₃ solution (20 mL) and extracted with EtOAc (2×10 mL). The combined organic layers were dried over anhydrous sodium sulfate, concentrated and purified with prep-HPLC [column: Waters Xbridge 150×25 mm×5 μm; A: water(NH₄HCO₃, B: ACN, B %:38%-68% B over 9 min] to afford the title compound (5.91 mg, 22% yield) as off-white solid; ¹H NMR (400 MHz, METHANOL-d₄) δ=7.99-7.90 (d, J=8.0 Hz, 1H), 7.28-7.17 (m, 1H), 7.04 (t, J=9.2 Hz, 1H), 5.44-5.17 (d, J=54.8 Hz, 1H), 4.37-4.25 (m, 2H), 4.25-4.10 (m, 1H), 4.07-3.94 (m, 1H), 3.84-3.69 (m, 2H), 3.47 (br d, J=11.6 Hz, 1H), 3.35 (br d, J=12.4 Hz, 2H), 3.26-3.21 (m, 1H), 3.21-3.15 (m, 1H), 3.11-2.95 (m, 1H), 2.4-2.15 (m, 3H), 2.10-1.85 (m, 7H); LCMS (ESI, M+1): m/z=719.2.

### Example 143A

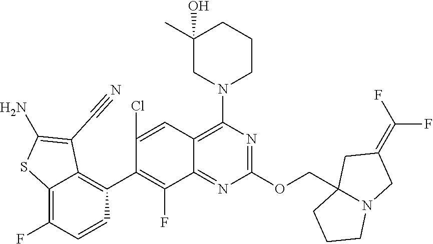

### 2-amino-4-(6-chloro-2-((2-(difluoromethylene)tetrahydro-1H-pyrrolizin-7a(5H)-yl)methoxy)-8-fluoro-4-((R)-3-hydroxy-3-methylpiperidin-1-yl)quinazolin-7-yl)-7-fluorobenzo[b]thiophene-3-carbonitrile

### Example 143B

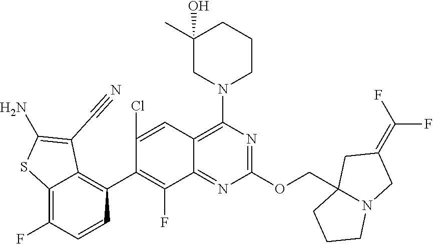

### 2-amino-4-(6-chloro-2-((2-(difluoromethylene)tetrahydro-1H-pyrrolizin-7a(5H)-yl)methoxy)-8-fluoro-4-((R)-3-hydroxy-3-methylpiperidin-1-yl)quinazolin-7-yl)-7-fluorobenzo[b]thiophene-3-carbonitrile

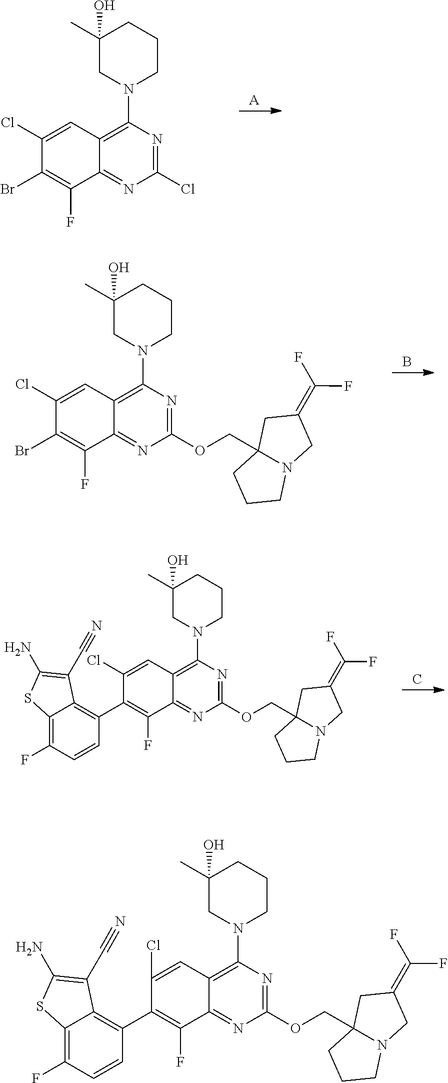

Step A. (3R)-1-(7-bromo-6-chloro-2-((2-(difluoromethylene)tetrahydro-1H-pyrrolizin-7a(5H)-yl)methoxy)-8-fluoroquinazolin-4-yl)-3-methylpiperidin-3-ol: To a mixture of (R)-1-(7-bromo-2,6-dichloro-8-fluoroquinazolin-4-yl)-3-methylpiperidin-3-ol (250 mg, 1.0 equiv), (2-(difluoromethylene)tetrahydro-1H-pyrrolizin-7a(5H)-yl)methanol (150 mg, 1.3 equiv), and Cs₂CO₃ (597 mg, 3.0 equiv) in DMF (2.5 mL) and THE (2.5 mL) was added DABCO (68.5 mg, 1.0 equiv). The reaction was stirred at 25° C. for 1 hour. The mixture was diluted with water (20 mL) and extracted with EtOAc (3×50 mL). The combined organic layers were washed with brine (30 mL), dried over anhydrous sodium sulfate, concentrated, and purified by reversed phase flash chromatography [C18, 0.1% formic acid condition] to afford the title compound (180 mg, 43% yield) as yellow oil; LCMS (ESI, M+1, M+3): m/z=561.1, 563.1.

Step B. tert-butyl (4-(6-chloro-2-((2-(difluoromethylene)tetrahydro-1H-pyrrolizin-7a(5H)-yl)methoxy)-8-fluoro-4-((R)-3-hydroxy-3-methylpiperidin-1-yl)quinazolin-7-yl)-3-cyano-7-fluorobenzo[b]thiophen-2-yl)carbamate: To a mixture of (3R)-1-(7-bromo-6-chloro-2-((2-(difluoromethylene)tetrahydro-1H-pyrrolizin-7a(5H)-yl)methoxy)-8-fluoroquinazolin-4-yl)-3-methylpiperidin-3-ol (140 mg, 1.0 equiv), tert-butyl N-[3-cyano-4-(5,5-dimethyl-1,3,2-dioxaborinan-2-yl)-7-fluoro-benzothiophen-2-yl]carbamate (201 mg, 2.0 equiv), and Cs₂CO₃ (243 mg, 3.0 equiv) in toluene (3 mL) was added Pd(DPEphos)Cl₂ (34.3 mg, 0.20 equiv). The reaction was degassed and purged with N₂ for 3 times. The reaction was stirred at 110° C. for 1 hour. The mixture was quenched with water (3 mL) and extracted with ethyl acetate (3×4 mL). The combined organic layers were dried over anhydrous sodium sulfate, concentrated, and purified with silica gel chromatography (petroleum ether/ethyl acetate=50/1 to 0/1) to afford the title compound (110 mg, crude) as yellow solid.

Step C. 2-amino-4-(6-chloro-2-((2-(difluoromethylene)tetrahydro-1H-pyrrolizin-7a(5H)-yl)methoxy)-8-fluoro-4-((R)-3-hydroxy-3-methylpiperidin-1-yl)quinazolin-7-yl)-7-fluorobenzo[b]thiophene-3-carbonitrile: To a solution of tert-butyl (4-(6-chloro-2-((2-(difluoromethylene)tetrahydro-1H-pyrrolizin-7a(5H)-yl)methoxy)-8-fluoro-4-((R)-3-hydroxy-3-methylpiperidin-1-yl)quinazolin-7-yl)-3-cyano-7-fluorobenzo[b]thiophen-2-yl)carbamate (80.0 mg, 1.0 equiv) in DCM (0.3 mL) was added TFA (767 mg, 65 equiv) at 0° C. The reaction was stirred at 0° C. for 0.5 hours. The mixture was basified with saturated aqueous NaHCO₃(2.0 mL) and extracted with ethyl acetate (3×2 mL). The combined organic layers were dried over anhydrous sodium sulfate, concentrated, and purified with prep-HPLC [column: Waters Xbridge 150×25 mm×5 μm; A: water (ammonia hydroxide v/v), B: ACN, B %: 53%-83% B over 10 min] to afford two peaks of the title compounds.

WX-96019A (8.52 mg, 12% yield) as white solid; ¹H NMR (400 MHz, METHANOL-d₄) δ=8.07 (s, 1H), 7.20 (dd, J=5.2, 8.4 Hz, 1H), 7.03 (dd, J=8.8, 9.6 Hz, 1H), 4.39-4.32 (m, 1H), 4.32-4.18 (m, 2H), 4.04 (br d, J=13.2 Hz, 1H), 3.83 (br d, J=13.6 Hz, 1H), 3.53 (d, J=13.6 Hz, 1H), 3.49-3.38 (m, 2H), 3.23-3.10 (m, 1H), 2.87-2.79 (m, 1H), 2.78-2.68 (m, 1H), 2.57-2.48 (m, 1H), 2.21-2.08 (m, 2H), 2.05-1.88 (m, 3H), 1.87-1.69 (m, 3H), 1.25 (s, 3H); LCMS (ESI, M+1): m/z=673.1; SFC: ChiralpakAD-3 50×4.6 mm I.D., 3 μm column A: 5%-40% IPA(0.05% DEA), B: C02, 3 mL/min, 220 nm, t_{R}: 2.127 and 2.162 min;

WX-96019B (8.26 mg, 11% yield) as white solid; ¹H NMR (400 MHz, METHANOL-d₄) δ=8.04 (s, 1H), 7.21 (dd, J=4.8, 8.4 Hz, 1H), 7.04 (dd, J=8.4, 9.6 Hz, 1H), 4.38-4.32 (m, 1H), 4.31-4.18 (m, 2H), 4.04 (br d, J=13.2 Hz, 1H), 3.82 (br d, J=14.4 Hz, 1H), 3.52 (dd, J=2.4, 13.8 Hz, 1H), 3.49-3.37 (m, 2H), 3.21-3.11 (m, 1H), 2.87-2.78 (m, 1H), 2.77-2.68 (m, 1H), 2.56-2.48 (m, 1H), 2.21-2.08 (m, 2H), 2.05-1.87 (m, 3H), 1.84-1.67 (m, 3H), 1.27 (s, 3H); LCMS (ESI, M+1): m/z=673.1; SFC: ChiralpakAD-3 50×4.6 mm I.D., 3 μm column A: 5%-40% IPA (0.05% DEA), B: C02, 3 mL/min, 220 nm, t_{R}: 1.913 min.

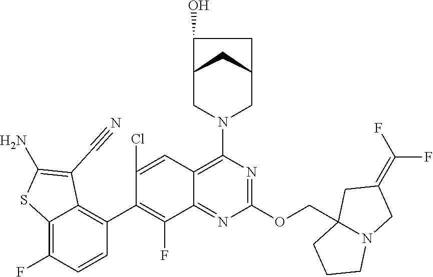 [SMILES: N#Cc1c(N)sc2c(F)ccc(-c3c(Cl)cc4c(N5C[C@@H]6C[C@H](C5)[C@H](O)C6)nc(OCC56CCCN5CC(=C(F)F)C6)nc4c3F)c12]

### 2-amino-4-(6-chloro-2-((2-(difluoromethylene)tetrahydro-1H-pyrrolizin-7a(5H)-yl)methoxy)-8-fluoro-4-((1R,5R,6R)-6-hydroxy-3-azabicyclo[3.2.1]octan-3-yl)quinazolin-7-yl)-7-fluorobenzo[b]thiophene-3-carbonitrile

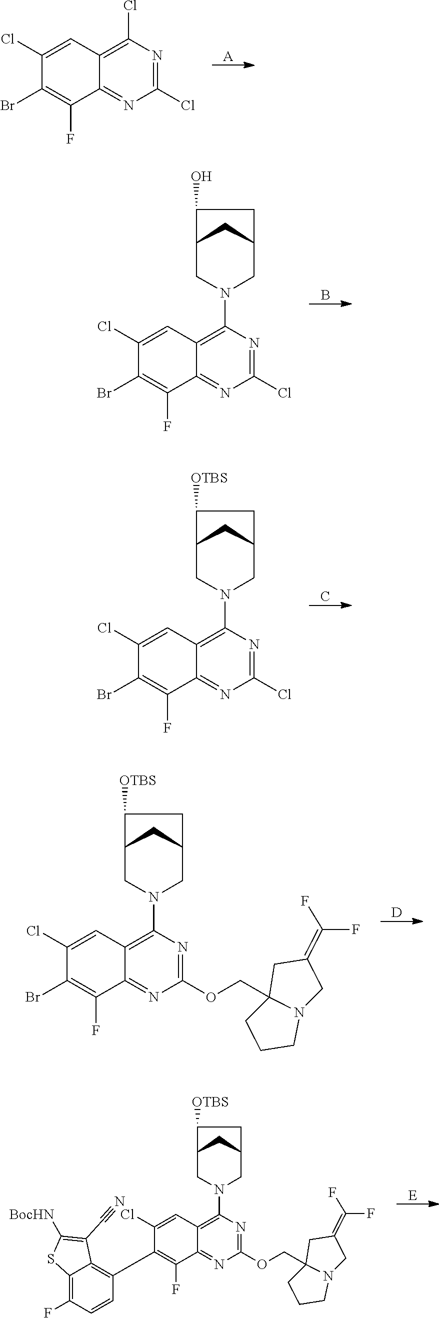

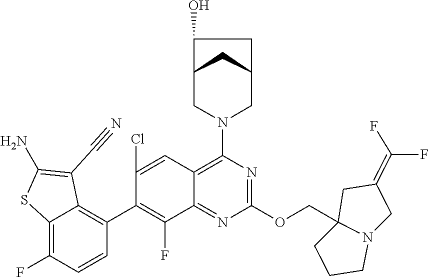 [SMILES: N#Cc1c(N)sc2c(F)ccc(-c3c(Cl)cc4c(N5C[C@@H]6C[C@H](C5)[C@H](O)C6)nc(OCC56CCCN5CC(=C(F)F)C6)nc4c3F)c12]

Step A. (1R,5R,6R)-3-(7-bromo-2,6-dichloro-8-fluoroquinazolin-4-yl)-3-azabicyclo[3.2.1]octan-6-ol: To a mixture of 7-bromo-2,4,6-trichloro-8-fluoroquinazoline (1.00 g, 1.0 equiv) and DIEA (1.17 g, 3.0 equiv) in DCM (15 mL) was added (1R,5R,6R)-3-azabicyclo[3.2.1]octan-6-ol (366 mg, 0.95 equiv) at −40° C. The reaction was stirred at −40° C. for 1 hour. The mixture was quenched with water (10 mL) and extracted with DCM (3×10 mL). The combined organic layers were dried over anhydrous sodium sulfate and concentrated to afford the title compound (1.25 g, 98% yield) as yellow solid.

Step B. 7-bromo-4-((1R,5R,6R)-6-((tert-butyldimethylsilyl)oxy)-3-azabicyclo[3.2.1]octan-3-yl)-2,6-dichloro-8-fluoroquinazoline: To a mixture of (1R,5R,6R)-3-(7-bromo-2,6-dichloro-8-fluoroquinazolin-4-yl)-3-azabicyclo[3.2.1]octan-6-ol (1.50 g, 1.0 equiv) and TBDMSCl (1.07 g, 2.0 equiv) in DMF (20 mL) were added imidazole (727 mg, 3.0 equiv) and DMAP (217 mg, 0.50 equiv). The reaction was stirred at 30° C. for 12 hours. The mixture was diluted with water (20 mL) and extracted with ethyl acetate (3×30 mL). The combined organic layers were washed with brine (3×40 mL), dried over anhydrous sodium sulfate, concentrated, and purified by silica gel chromatography (petroleum ether/ethyl acetate=50/1 to 10/1) to afford the title compound (1.80 g, 93% yield) as yellow solid; LCMS (ESI, M+1, M+3): m/z=533.8, 535.8.

Step C. 7-bromo-4-((1R,5R,6R)-6-((tert-butyldimethylsilyl)oxy)-3-azabicyclo[3.2.1]octan-3-yl)-6-chloro-2-((2-(difluoromethylene)tetrahydro-1H-pyrrolizin-7a(5H)-yl)methoxy)-8-fluoroquinazoline: To a mixture of 7-bromo-4-((1R,5R,6R)-6-((tert-butyldimethylsilyl)oxy)-3-azabicyclo[3.2.1]octan-3-yl)-2,6-dichloro-8-fluoroquinazoline (500 mg, 1.0 equiv) and Cs₂CO₃ (913 mg, 3.0 equiv) in DMF (5 mL) and THF (5 mL) were added DABCO (105 mg, 1.0 equiv) and (2-(difluoromethylene)tetrahydro-1H-pyrrolizin-7a(5H)-yl)methanol (265 mg, 1.5 equiv). The reaction was stirred at 20° C. for 12 hours. The mixture was diluted with water (8 mL) and extracted with ethyl acetate (3×8 mL). The combined organic layers were dried over anhydrous sodium sulfate, concentrated, and purified by reversed phase flash chromatography [C18, 0.1% formic acid condition] to afford the title compound (300 mg, 45% yield) as yellow solid; LCMS (ESI, M+1, M+3): m/z=687.1, 689.1.

Step D. tert-butyl (4-(4-((1R,5R,6R)-6-((tert-butyldimethylsilyl)oxy)-3-azabicyclo[3.2.1]octan-3-yl)-6-chloro-2-((2-(difluoromethylene)tetrahydro-1H-pyrrolizin-7a(5H)-yl)methoxy)-8-fluoroquinazolin-7-yl)-3-cyano-7-fluorobenzo[b]thiophen-2-yl)carbamate: To a mixture of 7-bromo-4-((1R,5R,6R)-6-((tert-butyldimethylsilyl)oxy)-3-azabicyclo[3.2.1]octan-3-yl)-6-chloro-2-((2-(difluoromethylene)tetrahydro-1H-pyrrolizin-7a(5H)-yl)methoxy)-8-fluoroquinazoline (270 mg, 1.0 equiv), tert-butyl N-[3-cyano-4-(5,5-dimethyl-1,3,2-dioxaborinan-2-yl)-7-fluoro-benzothiophen-2-yl]carbamate (254 mg, 1.6 equiv) and Cs₂CO₃ (383 mg, 3.0 equiv) in toluene (5 mL) added Pd(DPEphos)Cl₂ (54.0 mg, 0.20 equiv). The reaction was degassed and purged with nitrogen 3 times. The reaction was stirred at 110° C. for 1 hour under N₂ atmosphere. The mixture was diluted with water (5 mL) and extracted with ethyl acetate (3×5 mL). The combined organic layers were dried over anhydrous sodium sulfate, concentrated and purified by reversed phase flash chromatography [C18, 0.1% formic acid condition] to afford the title compound (200 mg, crude) as yellow solid.

Step E. 2-amino-4-(6-chloro-2-((2-(difluoromethylene)tetrahydro-1H-pyrrolizin-7a(5H)-yl)methoxy)-8-fluoro-4-((1R,5R,6R)-6-hydroxy-3-azabicyclo[3.2.1]octan-3-yl)quinazolin-7-yl)-7-fluorobenzo[b]thiophene-3-carbonitrile: A solution of tert-butyl (4-(4-((1R,5R,6R)-6-((tert-butyldimethylsilyl)oxy)-3-azabicyclo[3.2.1]octan-3-yl)-6-chloro-2-((2-(difluoromethylene)tetrahydro-1H-pyrrolizin-7a(5H)-yl)methoxy)-8-fluoroquinazolin-7-yl)-3-cyano-7-fluorobenzo[b]thiophen-2-yl)carbamate (160 mg, 1.0 equiv) in HCl.MeOH (4 M, 2 mL) was stirred at 25° C. for 1 hour. The mixture was filtered and purified by prep-HPLC [Phenomenex luna C18 150×25 mm×10 μm; A: water (FA); B: ACN; B %:25%-45% over 10 min] to afford the title compound (13.3 mg, 10% yield, 0.29 HCOOH) as off-white solid; ¹H NMR (400 MHz, METHANOL-d₄) δ=8.27 (d, J=1.2 Hz, 1H), 7.21 (dd, J=5.2, 8.4 Hz, 1H), 7.09-7.00 (m, 1H), 4.73 (br d, J=12 Hz, 1H), 4.69-4.60 (m, 1H), 4.48-4.30 (m, 3H), 4.04-3.93 (m, 1H), 3.66-3.57 (m, 2H), 3.52-3.43 (m, 1H), 2.93-2.83 (m, 2H), 2.61 (br d, J=16.0 Hz, 1H), 2.37 (br d, J=6.0 Hz, 1H), 2.30-2.17 (m, 3H), 2.14-1.91 (m, 4H), 1.90-1.77 (m, 2H), 1.48 (br dd, J=2.0, 14.0 Hz, 1H); LCMS (ESI, M+1): m/z=685.1.

### Example 145

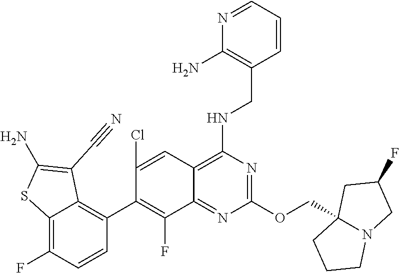 [SMILES: N#Cc1c(N)sc2c(F)ccc(-c3c(Cl)cc4c(NCc5cccnc5N)nc(OC[C@@]56CCCN5C[C@H](F)C6)nc4c3F)c12]

### 2-amino-4-(4-(((2-aminopyridin-3-yl)methyl)amino)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

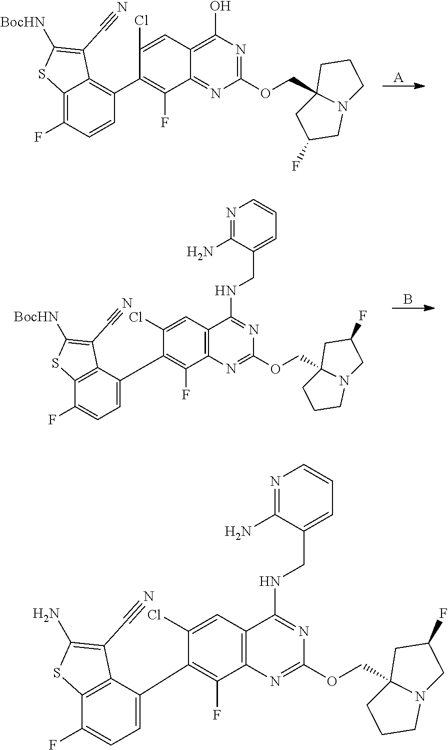

Step A. tert-butyl (4-(4-(((2-aminopyridin-3-yl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) and TEA (47.0 mg, 3.0 equiv) in DMSO (1 mL) was added and PyBOP (161 mg, 2.0 equiv). The reaction was stirred at 25° C. for 0.5 hours and then 3-(Aminomethyl)pyridin-2-amine (57.2 mg, 3.0 equiv) was added. The reaction was stirred at 25° C. for 2 hours. The mixture was diluted with water (10 mL) and extracted with EtOAc (2×10 mL). The combined organic layers were washed with brine (2×10 mL), dried over anhydrous sodium sulfate, concentrated and purified by prep-HPLC [Phenomenex luna C18 150×25 mm×10 μm; A: water (FA); B: ACN; B %:35%-65% over 9 min] to afford the title compound (34.0 mg, 29% yield) as yellow solid; LCMS (ESI, M+1): m/z=751.3.

Step B. 2-amino-4-(4-(((2-aminopyridin-3-yl)methyl)amino)-6-chloro-8-fluoro-2-(((2R,7aS)-2-fluorotetrahydro-1H-pyrrolizin-7a(5H)-yl)methoxy)guinazolin-7-yl)-7-fluorobenzo[b]thiophene-3-carbonitrile: To a solution of tert-butyl (4-(4-(((2-aminopyridin-3-yl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (130 mg) in DCM (1.5 mL) was added TFA (2.30 g). The reaction was stirred at 0° C. for 1 hour. The mixture was concentrated. The residue was diluted with water (1 mL), neutralized with solid NaHCO₃, and extracted with EtOAc (2×2 mL). The combined organic layers were dried over anhydrous sodium sulfate, concentrated and purified by prep-HPLC [Phenomenex luna C18 150×25 mm×10 μm; A: water (FA); B: ACN; B %: 5%-35% over 10 min] to afford the title compound (25.3 mg, 0.32 HCOOH) as off-white solid; ¹H NMR (400 MHz, METHANOL-d₄) δ=8.25-8.17 (m, 1H), 7.97-7.87 (m, 1H), 7.67-7.56 (m, 1H), 7.29-7.19 (m, 1H), 7.12-7.00 (m, 1H), 6.71 (dd, J=5.6, 7.6 Hz, 1H), 5.66-5.36 (m, 1H), 4.77-4.71 (m, 2H), 4.64-4.49 (m, 2H), 3.95-3.73 (m, 3H), 3.46-3.39 (m, 1H), 2.68-2.47 (m, 2H), 2.43-2.23 (m, 3H), 2.18-1.99 (m, 1H). LCMS (ESI, M+1): m/z=651.3.

### Example 146

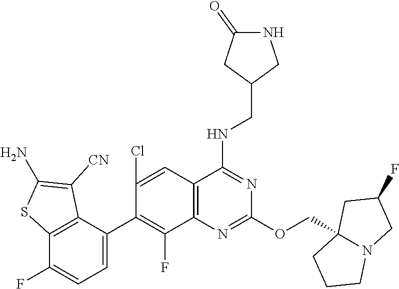 [SMILES: N#Cc1c(N)sc2c(F)ccc(-c3c(Cl)cc4c(NCC5CNC(=O)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(((5-oxopyrrolidin-3-yl)methyl)amino)quinazolin-7-yl)-7-fluorobenzo[b]thiophene-3-carbonitrile

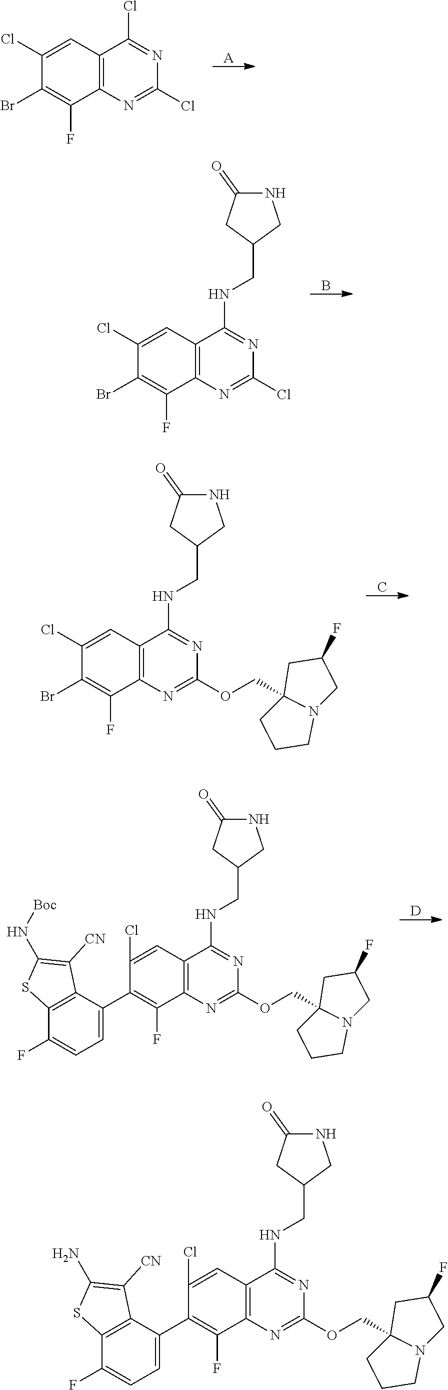

Step A. 4-(((7-bromo-2,6-dichloro-8-fluoroquinazolin-4-yl)amino)methyl)pyrrolidin-2-one: To a solution of 7-bromo-2,4,6-trichloro-8-fluoroquinazoline (980 mg, 1.0 equiv) and DIEA (1.15 g, 3.0 equiv) in DCM (10 mL) was added 4-(aminomethyl)pyrrolidin-2-one hydrochloride (450 mg, 1.0 equiv) at 0° C. The reaction was stirred at 20° C. for 2 hours. The mixture was concentrated and triturated with petroleum ether (30 mL) at 20° C. for 1 hour to afford the title compound (1.19 g, 95% yield) as yellow solid; LCMS (ESI, M+1, M+3): m/z=406.9, 408.9.

Step B. 4-(((7-bromo-6-chloro-8-fluoro-2-(((2R,7aS)-2-fluorotetrahydro-1H-pyrrolizin-7a(5H)-yl)methoxy)quinazolin-4-yl)amino)methyl)pyrrolidin-2-one: A mixture of 4-(((7-bromo-2,6-dichloro-8-fluoroquinazolin-4-yl)amino)methyl)pyrrolidin-2-one (600 mg, 1.0 equiv) in ((2R,7aS)-2-fluorotetrahydro-1H-pyrrolizin-7a(5H)-yl)methanol (1.87 g, 8.0 equiv) was stirred at 100° C. for 3 hours. The mixture was triturated with EtOAc (20 mL) and filtered to afford the title compound (520 mg, 56% yield) as off-white solid; LCMS (ESI, M+1, M+3): m/z=530.1, 532.1.

Step C. tert-butyl (4-(6-chloro-8-fluoro-2-(((2R,7aS)-2-fluorotetrahydro-1H-pyrrolizin-7a(5H)-yl)methoxy)-4-(((5-oxopyrrolidin-3-yl)methyl)amino)quinazolin-7-yl)-3-cyano-7-fluorobenzo[b]thiophen-2-yl)carbamate: To a mixture of 4-(((7-bromo-6-chloro-8-fluoro-2-(((2R,7aS)-2-fluorotetrahydro-1H-pyrrolizin-7a(5H)-yl)methoxy)quinazolin-4-yl)amino)methyl)pyrrolidin-2-one (350 mg, 1.0 equiv), tert-butyl (3-cyano-4-(5,5-dimethyl-1,3,2-dioxaborinan-2-yl)-7-fluorobenzo[b]thiophen-2-yl)carbamate (400 mg, 1.5 equiv), and Cs₂CO₃ (645 mg, 3.0 equiv) in THE (6 mL) was added Pd(DPEphos)Cl₂ (45.3 mg, 0.1 equiv). The reaction was degassed and purged with nitrogen 3 times. The reaction was stirred at 60° C. for 2 hours. The mixture was diluted with water (15 mL) and extracted with EtOAc (2×30 mL). The combined organic layers were dried over anhydrous sodium sulfate, concentrated, and purified with reversed phase flash chromatography [C18, 0.1% formic acid condition] to afford the title compound (205 mg, 28% yield) as yellow oil; LCMS (ESI, M+1): m/z=742.2.

Step D. 2-amino-4-(6-chloro-8-fluoro-2-(((2R,7aS)-2-fluorotetrahydro-1H-pyrrolizin-7a(5H)-yl)methoxy)-4-(((5-oxopyrrolidin-3-yl)methyl)amino)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(((5-oxopyrrolidin-3-yl)methyl)amino)quinazolin-7-yl)-3-cyano-7-fluorobenzo[b]thiophen-2-yl)carbamate (200 mg, 1.0 equiv) in DCM (0.5 mL) was added TFA (1.54 g, 50 equiv) at 0° C. The reaction was stirred at 20° C. for 0.5 hours. The mixture was diluted with DCM (5.0 mL) and sat. NaHCO₃(5.0 mL) was added. The organic layer was dried over anhydrous sodium sulfate, concentrated, and purified with prep-HPLC [column: Phenomenex luna C18 150×25 mm×10 μm; A: water (FA), B: ACN, B %: 12%-42% over 10 min] to afford the title compound (8.08 mg, 5% yield, HCOOH salt) as white solid; ¹H NMR (400 MHz, METHANOL-d₄) δ=8.14 (s, 1H), 7.20 (dd, J=5.2, 8.0 Hz, 1H), 7.11-6.95 (m, 1H), 5.52-5.34 (m, 1H), 4.57-4.35 (m, 2H), 3.85-3.69 (m, 2H), 3.69-3.42 (m, 4H), 3.30-3.18 (m, 3H), 3.07-2.92 (m, 1H), 2.57-2.49 (m, 1H), 2.48-2.37 (m, 1H), 2.37-2.10 (m, 4H), 2.09-1.94 (m, 1H); LCMS (ESI, M+1): m/z=642.2.

### Example 147

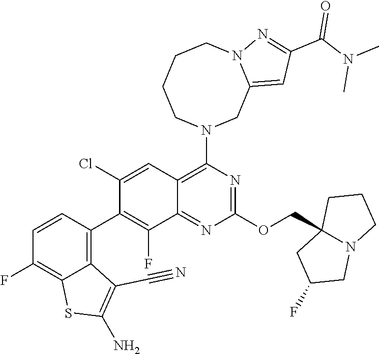 [SMILES: CN(C)C(=O)c1cc2n(n1)CCCCN(c1nc(OC[C@@]34CCCN3C[C@H](F)C4)nc3c(F)c(-c4ccc(F)c5sc(N)c(C#N)c45)c(Cl)cc13)C2]

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide

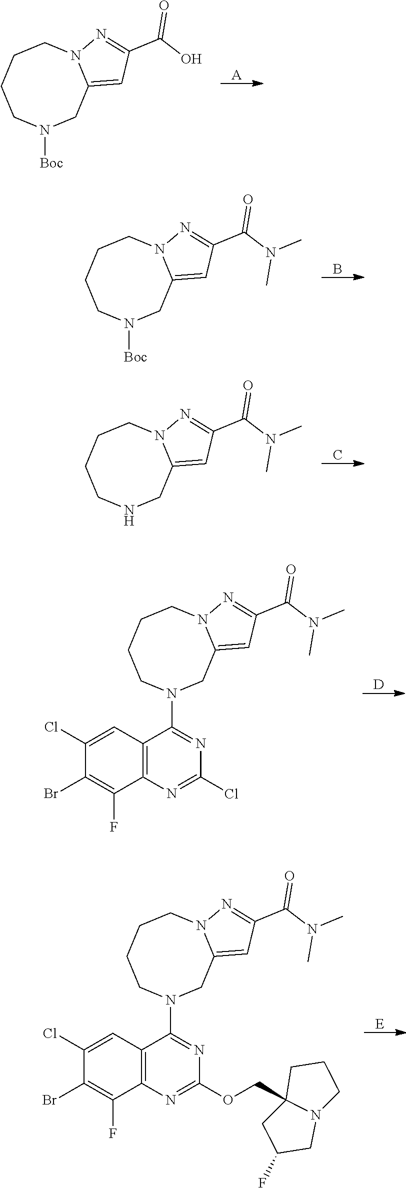

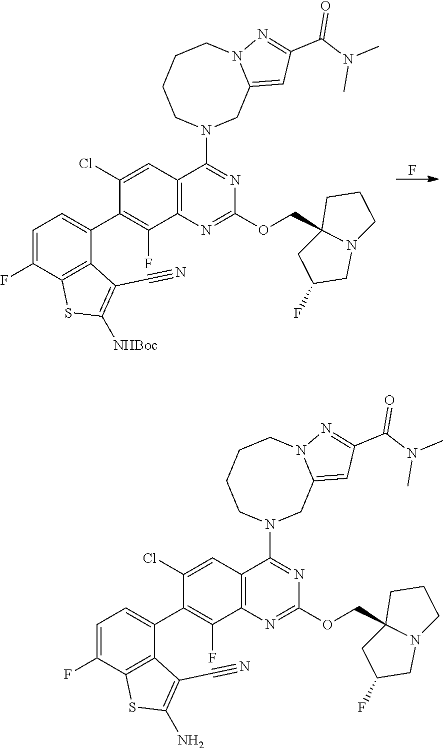

Step A. tert-butyl 2-(dimethylcarbamoyl)-6,7,8,9-tetrahydropyrazolo[1,5-a][1,4]diazocine-5(4H)-carboxylate. To a solution of 5-(tert-butoxycarbonyl)-4,5,6,7,8,9-hexahydropyrazolo[1,5-a][1,4]diazocine-2-carboxylic acid (300 mg, 1.0 equiv), DIEA (788 mg, 6.0 equiv) and HATU (1.16 g, 3.0 equiv) in DMF (3.0 mL) was added dimethylamine (2 M, 1.5 equiv). The reaction was stirred at 20° C. for 1 hour. The mixture was diluted with water (4.0 mL) and extracted with EtOAc (3×5.0 mL). The combined organic layers were washed with brine (2×5.0 mL), dried over anhydrous sodium sulfate, concentrated, and purified by reversed phase flash chromatography [C18, 0.1% formic acid condition] to afford the title compound (285 mg, 87% yield) as orange oil; LCMS (ESI, M+1): m/z=323.2.

Step B. N,N-dimethyl-4,5,6,7,8,9-hexahydropyrazolo[1,5-a][1,4]diazocine-2-carboxamide: A mixture of tert-butyl 2-(dimethylcarbamoyl)-6,7,8,9-tetrahydropyrazolo[1,5-a][1,4]diazocine-5(4H)-carboxylate (200 mg, 1.0 equiv) in HCl.dioxane (4 M, 2.00 mL, 13 equiv) was stirred at 20° C. for 0.5 hours. The mixture was concentrated. The residue was diluted with MeOH (2.0 mL). NaHCO₃(0.3 g) was added into the mixture. The mixture was stirred at 20° C. for 0.5 hour. The mixture was filtered and concentrated to afford the title compound (210 mg, crude) as white solid; LCMS (ESI, M+1): m/z=223.2.

Step C. 5-(7-bromo-2,6-dichloro-8-fluoroquinazolin-4-yl)-N,N-dimethyl-4,5,6,7,8,9-hexahydropyrazolo[1,5-a][1,4]diazocine-2-carboxamide: To a solution of 7-bromo-2,4,6-trichloro-8-fluoroquinazoline (168 mg, 1.0 equiv) and DIEA (395 mg, 6.0 equiv) in DCM (2.0 mL) was added N,N-dimethyl-4,5,6,7,8,9-hexahydropyrazolo[1,5-a][1,4]diazocine-2-carboxamide (170 mg, 1.5 equiv). The reaction was stirred at −40° C. for 0.5 hours. The mixture was quenched with water (2.0 mL) at 0° C. and extracted with DCM (3×5.0 mL). The combined organic layers were washed with brine (2×5.0 mL), dried over anhydrous sodium sulfate, and concentrated to afford the title compound (200 mg, 76% yield) as yellow solid; LCMS (ESI, M+1, M+3): m/z=514.9, 516.9.

Step D. 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide: To a solution of 5-(7-bromo-2,6-dichloro-8-fluoroquinazolin-4-yl)-N,N-dimethyl-4,5,6,7,8,9-hexahydropyrazolo[1,5-a][1,4]diazocine-2-carboxamide (200 mg, 1.0 equiv) and ((2R,7aS)-2-fluorotetrahydro-1H-pyrrolizin-7a(5H)-yl)methanol (56.2 mg, 1.0 equiv) in DMF (2.0 mL) and THE (2.0 mL) were added DABCO (43.5 mg, 1.0 equiv) and Cs₂CO₃ (379 mg, 3.0 equiv). The reaction was stirred at 25° C. for 1 hour. The mixture was diluted with water (2.0 mL) and extracted with EtOAc (3×5.0 mL). The combined organic layers were washed with brine (2×5.0 mL), dried over anhydrous sodium sulfate, concentrated, and purified by reversed phase flash chromatography [C18, 0.1% formic acid condition] to afford the title compound (110 mg, 43% yield) as yellow solid; LCMS (ESI, M+1, M+3): m/z=638.0, 640.1.

Step E. tert-butyl (4-(6-chloro-4-(2-(dimethylcarbamoyl)-6,7,8,9-tetrahydropyrazolo[1,5-a][1,4]diazocin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of 5-(7-bromo-6-chloro-8-fluoro-2-(((2R,7aS)-2-fluorotetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide (100 mg, 1.0 equiv), tert-butyl (3-cyano-4-(5,5-dimethyl-1,3,2-dioxaborinan-2-yl)-7-fluorobenzo[b]thiophen-2-yl)carbamate (94.9 mg, 1.5 equiv) and Cs₂CO₃ (153 mg, 3.0 equiv) in toluene (1.0 mL) was added Pd(DPEphos)Cl₂ (10.8 mg, 0.10 equiv). The reaction was degassed and purged with nitrogen 3 times. The reaction was stirred at 110° C. for 1 hour. The mixture was diluted with water (2.0 mL) and extracted with EtOAc (3×5.0 mL). The combined organic layers were washed with brine (2×3.0 mL), dried over anhydrous sodium sulfate, concentrated, and purified by reversed phase flash chromatography [C18, 0.1% formic acid condition] to afford the title compound (80.0 mg, 53% yield) as yellow solid; LCMS (ESI, M+1): m/z=850.7.

Step F. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide: To a solution of tert-butyl (4-(6-chloro-4-(2-(dimethylcarbamoyl)-6,7,8,9-tetrahydropyrazolo[1,5-a][1,4]diazocin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (70.0 mg, 1.0 equiv) in DCM (2.0 mL) was added TFA (1.54 g, 163 equiv). The reaction was stirred at 20° C. for 0.5 hours. The mixture was concentrated. The residue was diluted with saturated NaHCO₃ aqueous solution (2.0 mL). The mixture was extracted with EtOAc (3×5.0 mL). The combined organic layers were dried over anhydrous sodium sulfate, concentrated and purified by prep-HPLC [Welch Xtimate C18 150×25 mm×5 μm; A: water (FA), B: ACN, B %: 18%-38% over 9 min] to afford the title compound (23.2 mg, 35% yield, 0.33 HCOOH) as white solid; ¹H NMR (400 MHz, METHANOL-d₄) δ=7.88 (s, 1H), 7.20 (dd, J=5.2, 8.4 Hz, 1H), 7.04 (t, J=8.8 Hz, 1H), 6.65 (s, 1H), 5.48-5.29 (m, 1H), 5.23 (s, 2H), 4.45 (br s, 2H), 4.40-4.23 (m, 2H), 4.07 (br s, 2H), 3.58-3.38 (m, 3H), 3.34 (s, 3H), 3.16 (br d, J=4.8 Hz, 1H), 3.09 (s, 3H), 2.50-2.27 (m, 2H), 2.25-2.15 (m, 1H), 2.10 (br d, J=5.6 Hz, 2H), 1.99 (br s, 5H); LCMS (ESI, M+1): m/z=750.2.

### Example 148

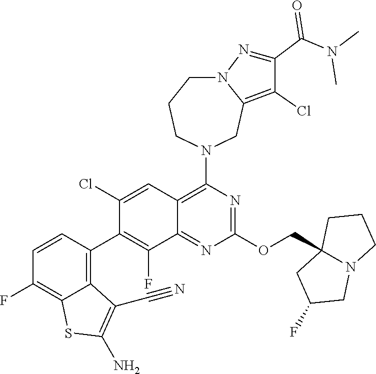 [SMILES: CN(C)C(=O)c1nn2c(c1Cl)CN(c1nc(OC[C@@]34CCCN3C[C@H](F)C4)nc3c(F)c(-c4ccc(F)c5sc(N)c(C#N)c45)c(Cl)cc13)CCC2]

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-chloro-N,N-dimethyl-5,6,7,8-tetrahydro-4H-pyrazolo[1,5-a][1,4]diazepine-2-carboxamide

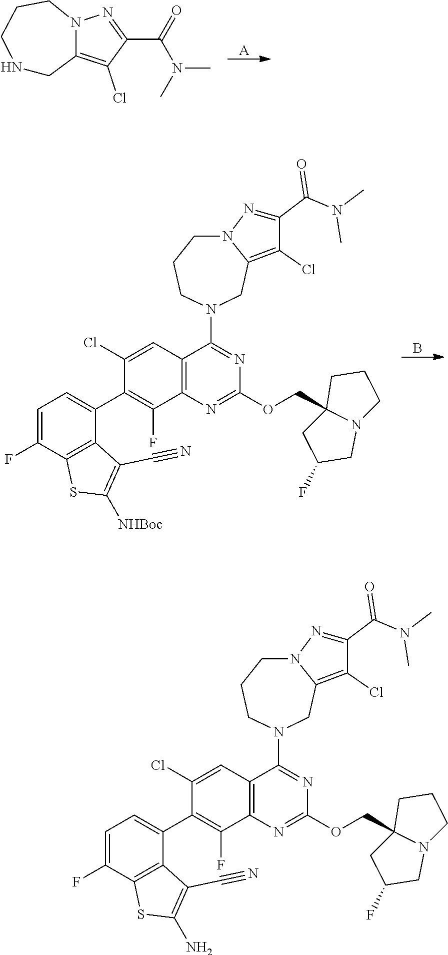

Step A. tert-butyl (4-(6-chloro-4-(3-chloro-2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)guinazolin-7-yl)-3-cyano-7-fluorobenzo[b]thiophen-2-yl)carbamate: A mixture of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (60 mg, 1.0 equiv), TEA (28.2 mg, 3.0 equiv) and PYBOP (72.5 mg, 1.5 equiv) in DMSO (1 mL) with stirred at 20° C. for 0.5 hour. Then 3-chloro-N,N-dimethyl-5,6,7,8-tetrahydro-4H-pyrazolo[1,5-a][1,4]diazepine-2-carboxamide (63.1 mg, 2.8 equiv) was added. The reaction was stirred at 20° C. for 1 hour. The mixture was quenched with water (2.0 mL) and purified by reversed phase flash chromatography [C18, 0.1% formic acid condition] to afford the title compound (80.0 mg, 94% yield) as a yellow solid; LCMS (ESI, M+1): m/z=870.4.

Step B. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-3-chloro-N,N-dimethyl-5,6,7,8-tetrahydro-4H-pyrazolo[1,5-a][1,4]diazepine-2-carboxamide: To a solution of tert-butyl (4-(6-chloro-4-(3-chloro-2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (50.0 mg, 1.0 equiv) in DCM (3.0 mL) was added TFA (2.30 g) at 0° C. The reaction was stirred at 20° C. for 1 hour. The mixture was concentrated and purified by prep-HPLC [Phenomenex luna C18 150×25 mm×10 μm; A:water (FA), B: ACN; 20%-50% over 10 min] to afford the title compound (14.6 mg, 32% yield, 0.338 HCOOH) as white solid; ¹H NMR (400 MHz, METHANOL-d₄) δ=7.92 (d, J=1.2 Hz, 1H), 7.24-7.17 (m, 1H), 7.08-7.00 (m, 1H), 5.48-5.26 (m, 1H), 5.13-4.98 (m, 2H), 4.41 (br d, J=9.6 Hz, 2H), 4.36-4.24 (m, 4H), 3.59-3.33 (m, 4H), 3.14 (s, 3H), 3.11 (s, 3H), 2.50-2.33 (m, 3H), 2.33-2.16 (m, 2H), 2.12-1.92 (m, 3H); LCMS (ESI, M+1): m/z=770.4.

### Example 149

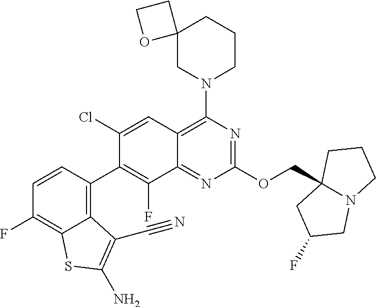 [SMILES: N#Cc1c(N)sc2c(F)ccc(-c3c(Cl)cc4c(N5CCCC6(CCO6)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-7-fluorobenzo[b]thiophene-3-carbonitrile

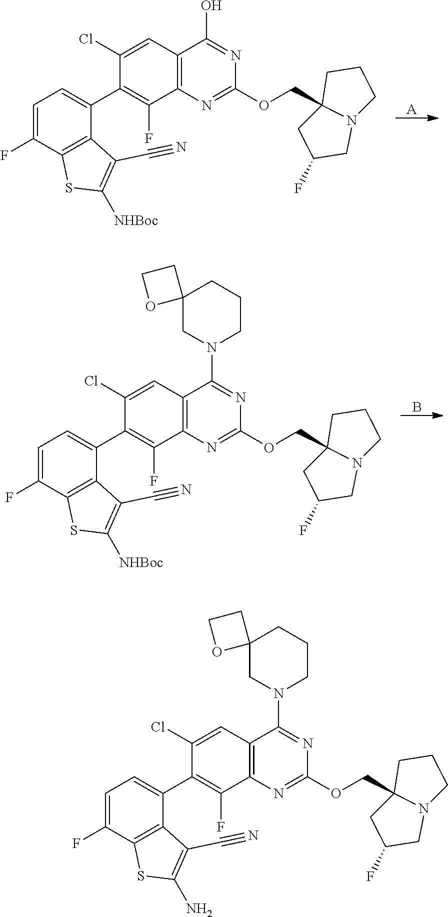

Step A. tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 equiv) and TEA (23.5 mg, 3.0 equiv) in DMSO (0.5 mL) was added PYBOP (60.4 mg, 1.5 equiv). The reaction was stirring at 25° C. for 0.5 hour, and then 1-oxa-8-azaspiro[3.5]nonane (19.7 mg, 2.0 equiv, oxalic acid) was added. The reaction was stirred at 25° C. for 2 hours. The mixture was diluted with water (5.0 mL) and extracted with EtOAc (2×3.0 mL). The combined organic layers were dried over anhydrous sodium sulfate, concentrated, and purified by silica gel chromatography (dichloromethane/methanol=10/1) to afford the title compound (40.0 mg, 68% yield) as yellow solid; LCMS (ESI, M+1): m/z=755.5.

Step B. 2-amino-4-(6-chloro-8-fluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.5]nonan-6-yl)quinazolin-7-yl)-3-cyano-7-fluorobenzo[b]thiophen-2-yl)carbamate (88.0 mg, 1.0 equiv) in DCM (0.67 mL) was added TFA (507 mg, 38.1 equiv). The reaction was stirred at 0° C. for 3 hours. The mixture was diluted with saturated TEA (2.0 mL) at −40° C. The mixture was concentrated and purified by prep-HPLC [waters xbridge C18 150×50 mm×10 μm; A: water (NH₄HCO₃); B: ACN; B %: 48%-78% over 10 min] to afford the title compound (16.0 mg, 21% yield) as white solid; ¹H NMR (400 MHz, DMSO-d₆) δ=8.24-7.93 (m, 3H), 7.32-7.21 (m, 1H), 7.15 (s, 1H), 5.38-5.14 (m, 1H), 4.53-4.35 (m, 2H), 4.23-3.86 (m, 4H), 3.73-3.45 (m, 1H), 3.18-2.96 (m, 4H), 2.86-2.77 (m, 1H), 2.35-2.25 (m, 2H), 2.17-1.98 (m, 4H), 1.89-1.68 (m, 6H); LCMS (ESI, M+1): m/z=655.3.

### Example 150

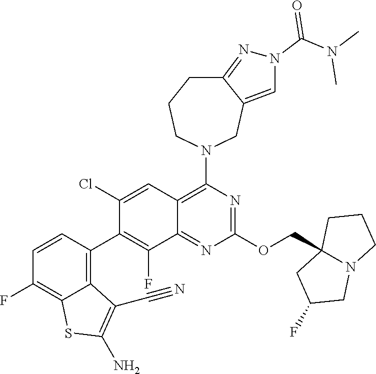 [SMILES: CN(C)C(=O)n1cc2c(n1)CCCN(c1nc(OC[C@@]34CCCN3C[C@H](F)C4)nc3c(F)c(-c4ccc(F)c5sc(N)c(C#N)c45)c(Cl)cc13)C2]

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide

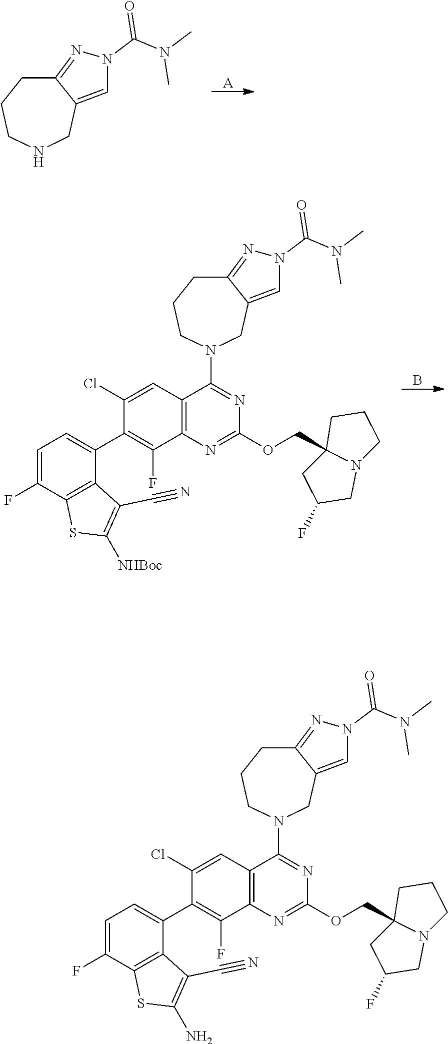

Step A. tert-butyl (4-(6-chloro-4-(2-(dimethylcarbamoyl)-2,6,7,8-tetrahydropyrazolo[4,3-c]azepin-5(4H)-yl)-8-fluoro-2-(((2R,7aS)-2-fluorotetrahydro-1H-pyrrolizin-7a(5H)-yl)methoxy) quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (60.0 mg 1.0 equiv) and TEA (28.2 mg, 3 equiv) in DMSO (1 mL) was added PYBOP (72.5 mg, 1.5 equiv). The reaction was stirred at 25° C. for 0.5 hour and N,N-dimethyl-5,6,7,8-tetrahydropyrazolo[4,3-c]azepine-2(4H)-carboxamide (38.7 mg, 2 equiv) was added. The reaction was stirred at 25° C. for 1.5 hours. The mixture was diluted by water (10.0 mL) and extracted with EtOAc (3×10.0 mL). The combined organic phase was dried over anhydrous sodium sulfate, concentrated, and purified by reversed phase flash chromatography [C18, 0.1% formic acid condition] to afford the title compound (58.0 mg, 72% yield) as yellow solid; LCMS (ESI, M+1): m/z=836.5.

Step B. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: To a solution of tert-butyl (4-(6-chloro-4-(2-(dimethylcarbamoyl)-2,6,7,8-tetrahydropyrazolo[4,3-c]azepin-5(4H)-yl)-8-fluoro-2-(((2R,7aS)-2-fluorotetrahydro-1H-pyrrolizin-7a(5H)-yl)methoxy) quinazolin-7-yl)-3-cyano-7-fluorobenzo[b]thiophen-2-yl)carbamate (53.0 mg, 1.0 equiv) in DCM (1.5 mL) was added TFA (0.80 mL, 169 equiv). The reaction was stirred at 25° C. for 0.5 hours. The mixture was concentrated and purified by prep-HPLC [Welch Xtimate C18 150×25 mm×5 μm; A: water(FA), B: ACN; B %: 25%-55% over 10 min] to afford the title compound (17.1 mg, 35% yield, 0.096 FA) as white solid; ¹H NMR (400 MHz, CD₃OD-d4) δ=8.16 (s, 1H), 8.01 (s, 1H), 7.24-7.14 (m, 1H), 7.03 (t, J=8.8 Hz, 1H), 5.45-5.25 (m, 1H), 4.97 (s, 2H), 4.38-4.21 (m, 4H), 3.52-3.35 (m, 2H), 3.24-3.15 (m, 6H), 3.14-3.03 (m, 2H), 3.01-2.92 (m, 2H), 2.29 (br d, J=3.6 Hz, 5H), 2.11-1.99 (m, 2H), 1.97-1.83 (m, 1H); LCMS (ESI, M+1): m/z=736.5.

### Example 151

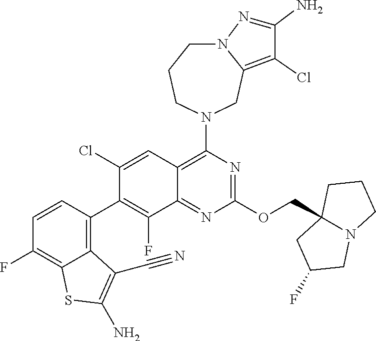 [SMILES: N#Cc1c(N)sc2c(F)ccc(-c3c(Cl)cc4c(N5CCCn6nc(N)c(Cl)c6C5)nc(OC[C@@]56CCCN5C[C@H](F)C6)nc4c3F)c12]

### 2-amino-4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

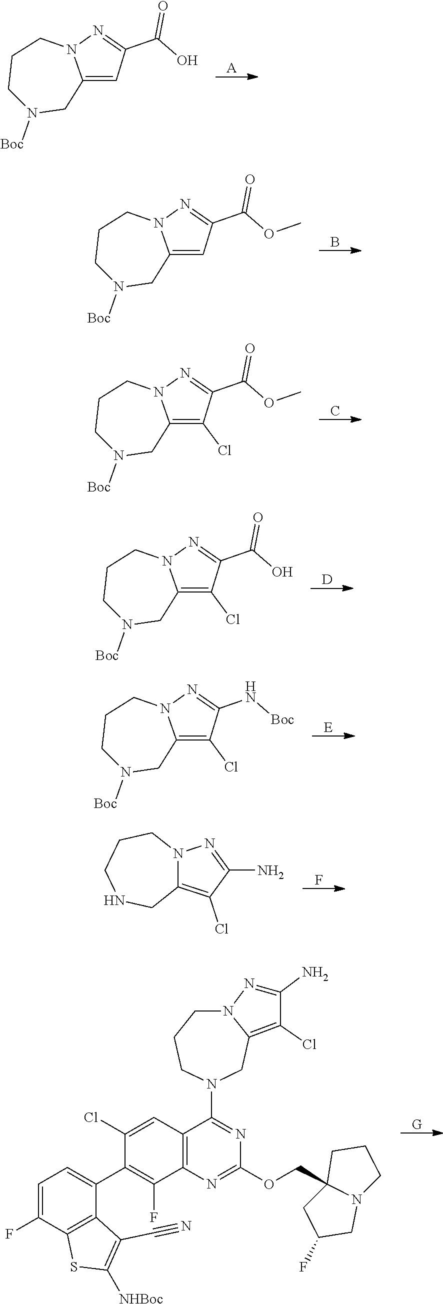

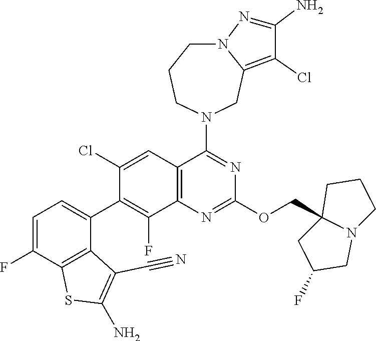 [SMILES: N#Cc1c(N)sc2c(F)ccc(-c3c(Cl)cc4c(N5CCCn6nc(N)c(Cl)c6C5)nc(OC[C@@]56CCCN5C[C@H](F)C6)nc4c3F)c12]

Step A. 5-(tert-butyl) 2-methyl 7,8-dihydro-4H-pyrazolo[1,5-a][1,4]diazepine-2,5(6H)-dicarboxylate: To a solution of 5-(tert-butoxycarbonyl)-5,6,7,8-tetrahydro-4H-pyrazolo[1,5-a][1,4]diazepine-2-carboxylic acid (5.00 g, 1.0 equiv) in dichloromethane (100 mL) in methanol (50.0 mL) was added dropwise TMSCHN₂ (2 M, 26.7 mL, 3.0 equiv) at 0° C. The reaction was stirred at 0° C. for 2.5 hours. The reaction was quenched with AcOH (4 mL, added dropwise). The mixture was concentrated. The residue was dissolved in dichloromethane (50.0 mL), washed with brine (20.0 mL), dried over anhydrous sodium sulfate, filtered, and concentrated to afford the title compound (5.36 g, crude) as a white solid. LCMS (ESI, M+1): m/z=296.1.

Step B. 5-(tert-butyl) 2-methyl 3-chloro-7,8-dihydro-4H-pyrazolo[1,5-a][1,4]diazepine-2,5(6H)-dicarboxylate: To a solution of 5-(tert-butyl) 2-methyl 7,8-dihydro-4H-pyrazolo[1,5-a][1,4]diazepine-2,5(6H)-dicarboxylate (5.60 g, 1.0 equiv) in acetic acid (26.0 mL) was added NCS (3.04 g, 1.2 equiv). The reaction was stirred at 80° C. for 0.5 hours. The mixture was concentrated. The residue was diluted with water (100 mL), neutralized with NaHCO₃(5.0 g) and extracted with EtOAc (3×200 mL). The combined organic phase was washed with brine (3×400 mL), dried over anhydrous sodium sulfate, filtered, and concentrated to afford the title compound (7.24 g, crude) as a yellow oil; LCMS (ESI, M+1): m/z=330.2

Step C. 5-(tert-butoxycarbonyl)-3-chloro-5,6,7,8-tetrahydro-4H-pyrazolo[1,5-a][1,4]diazepine-2-carboxylic acid: To a solution of 5-(tert-butyl) 2-methyl 3-chloro-7,8-dihydro-4H-pyrazolo[1,5-a][1,4]diazepine-2,5(6H)-dicarboxylate (7.14 g, 1.0 equiv) in methanol (35.0 mL) was added a solution of LiOH.H₂O (2.73 g, 3.0 equiv) in water (35.0 mL). The reaction was stirred at 20° C. for 2 hours. The mixture was extracted with EtOAc (3×10.0 mL) to remove the impurities. The aqueous phase was cooled to 0° C., acidifed with hydrochloric acid (1 M) and extracted with EtOAc (3×100 mL). The combined organic phase was washed with brine (3×100 mL), dried over anhydrous sodium sulfate, filtered and concentrated to afford the title compound (4.8 g, 70% yield) as a yellow solid; ¹H NMR (400 MHz, DMSO-d₆) δ=12.95 (s, 1H), 4.53 (br s, 2H), 4.49-4.43 (m, 2H), 3.66 (br s, 2H), 1.81 (br s, 2H), 1.36 (s, 9H); LCMS (ESI, M+1): m/z=316.2

Step D. tert-butyl 2-((tert-butoxycarbonyl)amino)-3-chloro-7,8-dihydro-4H-pyrazolo[1,5-a][1,4]diazepine-5(6H)-carboxylate: A mixture of 5-(tert-butoxycarbonyl)-3-chloro-5,6,7,8-tetrahydro-4H-pyrazolo[1,5-a][1,4]diazepine-2-carboxylic acid (1.00 g, 1.0 equiv), DPPA (1.31 g, 1.5 equiv), TEA (961 mg, 3.0 equiv) and 4 Å molecular sieve (100 mg, 1.0 equiv) in t-BuOH (20.0 mL) was stirred at 100° C. for 5 hours under nitrogen atmosphere. The mixture was filtered, concentrated under reduced pressure and purified by column chromatography (SiO₂, petroleum ether/ethyl acetate=1/0 to 1/1) to afford the title compound (950 mg, 75% yield) as a white solid; ¹H NMR (400 MHz, CDCl₃) δ=6.27 (br s, 1H), 4.45 (br s, 2H), 4.40-4.30 (m, 2H), 3.71 (br s, 2H), 1.94 (br s, 2H), 1.52 (s, 9H), 1.44 (s, 9H); LCMS (ESI, M+1): m/z=387.2.

Step E. 3-chloro-5,6,7,8-tetrahydro-4H-pyrazolo[1,5-a][1,4]diazepin-2-amine: To a solution of tert-butyl 2-((tert-butoxycarbonyl)amino)-3-chloro-7,8-dihydro-4H-pyrazolo[1,5-a][1,4]diazepine-5(6H)-carboxylate (341 mg, 1.0 equiv) in MeOH (3.41 mL) was added HCl.MeOH (4 M, 10.2 mL) at 0° C. The reaction was stirred at 20° C. for 2 hours. The mixture was concentrated. The residue was dissolved in MeOH (5.0 mL) and neutralized with NaHCO₃(0.2 g). The mixture was filtered and concentrated to afford the title compound (350 mg, crude) as a yellow solid; LCMS (ESI, M+1): m/z=187.4.

Step F. tert-butyl (4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-(6-chloro-8-fluoro-2-(((2R,7aS)-2-fluorotetrahydro-1H-pyrrolizin-7a(5H)-yl)methoxy)-5-hydroxyquinazolin-7-yl)-3-cyano-7-fluorobenzo[b]thiophen-2-yl)carbamate (50 mg, 1.0 equiv) in DMSO (0.2 mL) was added TEA (23.5 mg, 3.0 equiv) and PyBOP (60.4 mg, 1.5 equiv). The reaction was stirred at 20° C. for 0.5 hours and 3-chloro-5,6,7,8-tetrahydro-4H-pyrazolo[1,5-a][1,4]diazepin-2-amine (40.4 mg, 2.8 equiv) was added. The reaction was stirred at 20° C. for 0.5 hours. The mixture was quenched with water (2.0 mL) at 20° C. and purified by reversed phase flash chromatography [C18, 0.1% formic acid condition] to afford the title compound (80.0 mg, 98% yield) as a yellow solid; LCMS (ESI, M+1): m/z=814.2.

Step G. 2-amino-4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (50 mg, 1.0 equiv) in DCM (3.0 mL) was added TFA (2.30 g, 329 equiv) at 0° C. The reaction was stirred at 20° C. for 1 hour. The mixture was concentrated under reduced pressure and purified by reversed phase flash chromatography [C18, 0.1% formic acid condition] to afford the title compound (14.8 mg, 33% yield, 0.28 HCOOH) as a white solid; ¹H NMR (400 MHz, CD₃OD-d4) δ=7.92 (d, J=1.6 Hz, 1H), 7.20 (dd, J=5.2, 8.4 Hz, 1H), 7.04 (dd, J=8.4, 9.6 Hz, 1H), 5.49-5.26 (m, 1H), 5.01-4.91 (m, 2H), 4.35-4.21 (m, 4H), 4.16-4.10 (m, 2H), 3.57-3.34 (m, 3H), 3.18-3.06 (m, 1H), 2.49-2.18 (m, 5H), 2.14-1.91 (m, 3H); LCMS (ESI, M+1): m/z=714.2.

### Example 152

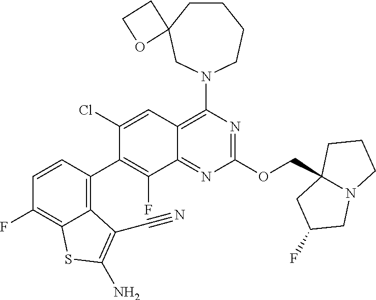 [SMILES: N#Cc1c(N)sc2c(F)ccc(-c3c(Cl)cc4c(N5CCCCC6(CCO6)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-7-fluorobenzo[b]thiophene-3-carbonitrile

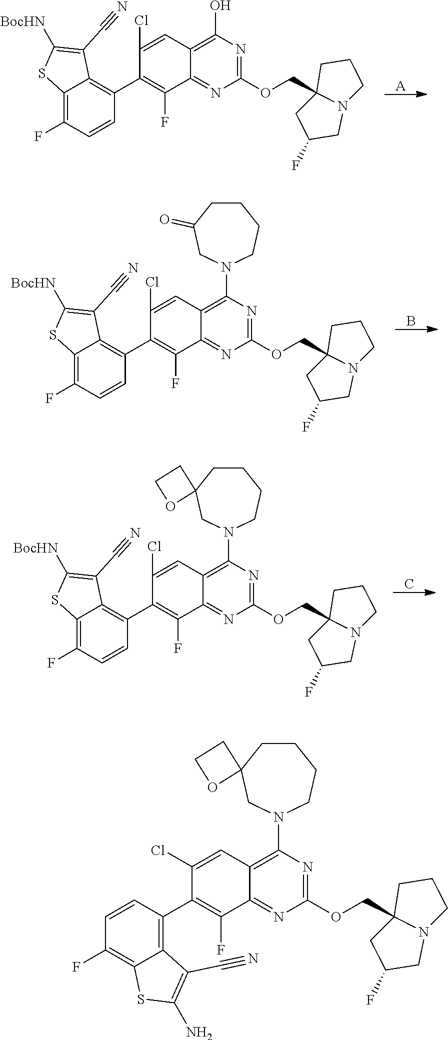

Step A. tert-butyl (4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (150 mg, 1.0 equiv) in DMSO (1.0 mL) were added TEA (70 mg, 3.0 equiv) and PyBOP (181 mg, 1.5 equiv). The reaction was stirred at 30° C. for 0.5 hours and then azepan-3-one (105 mg, 2.0 equiv, TFA salt) was added. The reaction was stirred at 30° C. for 12 hours. The mixture was quenched with water (30 mL) at 20° C. and extracted with EtOAc (2×60 mL). The combined organic layers were washed with brine (50 mL), dried over anhydrous sodium sulfate, concentrated, and purified with prep-TLC (SiO₂, DCM/MeOH=10/1) to afford the title compound (38 mg, 22% yield) as yellow solid; LCMS (ESI, M+1): m/z=741.4.

Step B. tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate: To a solution of trimethylsulfoxonium iodide (44.5 mg, 3.0 equiv) in t-BuOH (1.0 mL) was added t-BuOK (0.2 mL, 1M, 3.0 equiv) at 20° C. The reaction was stirred at 50° C. for 1.5 hours. Then a solution of tert-butyl (4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate (50.0 mg, 1.0 equiv) in t-BuOH (1.0 mL) was added. The reaction was stirred at 50° C. for 12 hours. The mixture was quenched with water (10 mL) at 20° C. and extracted with EtOAc (2×20 mL). The combined organic layers were washed with brine (10 mL), dried over anhydrous sodium sulfate, concentrated, and purified with prep-TLC (SiO₂, DCM/MeOH=10/1) to afford the title compound (5.0 mg, 9.6% yield) as yellow solid; LCMS (ESI, M+1): m/z=769.2.

Step C. 2-amino-4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate (8.0 mg, 1.0 equiv) in DCM (1 mL) was added TFA (767 mg, 517 equiv) at 0° C. The reaction was stirred at 0° C. for 6 hours. The mixture was quenched with TEA (1.5 mL) at −40° C., diluted with water (10 mL) and extracted with EtOAc (2×20 mL). The combined organic layers were washed with brine (10 mL), dried over anhydrous sodium sulfate, concentrated and purified with prep-HPLC (column: Waters Xbridge 150*25 mm*5 um; mobile phase: [water(NH₄HCO₃)−ACN];gradient:62%-92% B over 9 min) to afford the title compound (2 mg, 23% yield) as white solid; ¹H NMR (400 MHz, METHANOL-d₄) δ=8.50 (s, 1H), 8.11 (s, 2H), 7.22 (dd, J=5.2, 8.4 Hz, 1H), 7.17-7.12 (m, 1H), 6.56 (s, 1H), 5.39-5.25 (m, 1H), 4.65-4.35 (m, 1H), 4.35-3.97 (m, 4H), 3.63-3.59 (m, 2H), 2.75-3.10 (m, 4H), 2.50-2.26 (m, 8H), 1.9-1.69 (m, 7H); LCMS (ESI, M+1): m/z=669.2.

### Example 153

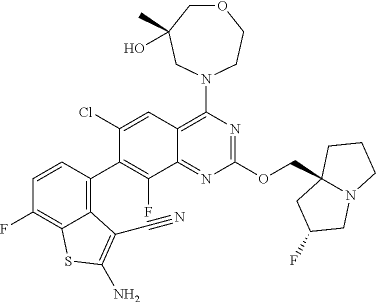 [SMILES: C[C@@]1(O)COCCN(c2nc(OC[C@@]34CCCN3C[C@H](F)C4)nc3c(F)c(-c4ccc(F)c5sc(N)c(C#N)c45)c(Cl)cc23)C1]

### 2-amino-4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile

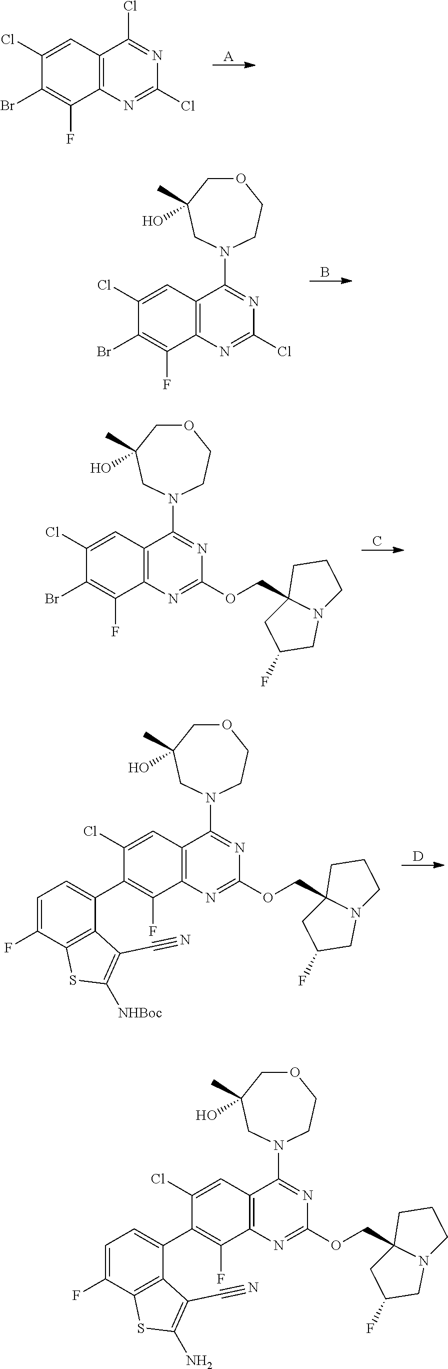

Step A. (S)-4-(7-bromo-2,6-dichloro-8-fluoroquinazolin-4-yl)-6-methyl-1,4-oxazepan-6-ol: To a solution of 7-bromo-2,4,6-trichloro-8-fluoroquinazoline (500 mg, 1.0 equiv) and DIEA (587 mg, 3.0 equiv) in DCM (5.0 mL) was added (S)-6-methyl-1,4-oxazepan-6-ol (254 mg, 1.0 equiv, HCl). The reaction was stirred at −40° C. for 1 hour. The mixture was diluted with water (10.0 mL) and extracted with DCM (3×15.0 mL). The combined organic layers were dried over anhydrous sodium sulfate, filtered, and concentrated to afford the title compound (657 mg, 97% yield) as yellow solid; LCMS (ESI, M+1, M+3): m/z=424.1, 426.1.

Step B. (S)-4-(7-bromo-6-chloro-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol: To a solution of (S)-4-(7-bromo-2,6-dichloro-8-fluoroquinazolin-4-yl)-6-methyl-1,4-oxazepan-6-ol (900 mg, 1.0 equiv) and ((2R,7aS)-2-fluorotetrahydro-1H-pyrrolizin-7a(5H)-yl)methanol (506 mg, 1.5 equiv) in THE (5.0 mL) and DMF (5.0 mL) were added Cs₂CO₃ (2.07 g, 3.0 equiv) and DABCO (238 mg, 1.0 equiv). The reaction was stirred at 25° C. for 2 hours. The mixture was diluted with water (20.0 mL) and extracted with EtOAc (2×30.0 mL). The combined organic layers were dried over anhydrous sodium sulfate, filtered, concentrated, and purified by column chromatography (SiO₂ petroleum ether/ethyl acetate=100/0 to 10/1) to afford the title compound (820 mg, 69% yield) as yellow solid; LCMS (ESI, M+1, M+3): m/z=547.1, 549.0.

Step C. tert-butyl (4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate: To a mixture of (S)-4-(7-bromo-6-chloro-8-fluoro-2-(((2R,7aS)-2-fluorotetrahydro-1H-pyrrolizin-7a(5H)-yl)methoxy)quinazolin-4-yl)-6-methyl-1,4-oxazepan-6-ol (150 mg, 1.0 equiv) and tert-butyl (3-cyano-4-(5,5-dimethyl-1,3,2-dioxaborinan-2-yl)-7-fluorobenzo[b]thiophen-2-yl)carbamate (111 mg, 1.0 equiv) in toluene (1.5 mL) were added Cs₂CO₃ (268 mg, 3.0 equiv) and Pd(DPEPhos)Cl₂ (18.8 mg, 0.1 equiv). The reaction was stirred at 110° C. for 1 hour. The mixture was diluted with water (5.0 mL) and extracted with EtOAc (3×5.0 mL). The combined organic layers were dried over anhydrous sodium sulfate, concentrated, and purified by reversed phase flash chromatography [C18, 0.1% formic acid condition] to afford the title compound (78.0 mg, 33% yield) as yellow solid; LCMS (ESI, M+1): m/z=759.5.

Step D. 2-amino-4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate (60.0 mg, 1.0 equiv) in DCM (0.8 mL) was added TFA (614 mg, 68.1 equiv). The reaction was stirred at 25° C. for 1 hour. The mixture was concentrated. The residue was diluted with NaHCO₃ aqueous solution (1.0 mL) and extracted with EtOAc (3×5.0 mL). The combined organic layers were dried over anhydrous sodium sulfate, concentrated, and purified by prep-HPLC [Phenomenex luna C18 150×25 mm×10 μm; A: water (FA); B: ACN; B %: 18%-48% over 10 min] to afford the title compound (34.3 mg, 65% yield) as white solid; ¹H NMR (400 MHz, DMSO-d₆) δ=8.62-8.46 (m, 1H), 8.19-8.08 (m, 2H), 7.31-7.09 (m, 2H), 5.42-5.14 (m, 2H), 4.32-4.17 (m, 1H), 4.15-4.05 (m, 2H), 4.03-3.91 (m, 3H), 3.89-3.67 (m, 2H), 3.55 (br d, J=5.6 Hz, 2H), 3.09 (br d, J=8.4 Hz, 2H), 3.01 (br s, 1H), 2.88-2.78 (m, 1H), 2.15-1.98 (m, 3H), 1.89-1.73 (m, 3H), 1.14 (br d, J=8.4 Hz, 3H); LCMS (ESI, M+1): m/z=659.3.

### Example 154

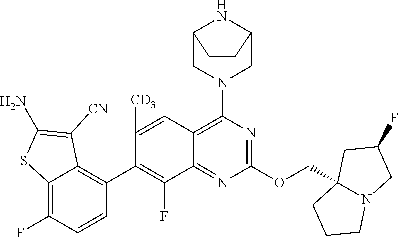 [SMILES: [2H]C([2H])([2H])c1cc2c(N3CC4CCC(C3)N4)nc(OC[C@@]34CCCN3C[C@H](F)C4)nc2c(F)c1-c1ccc(F)c2sc(N)c(C#N)c12]

### 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(methyl-d3)quinazolin-7-yl)-2-amino-7-fluorobenzo[b]thiophene-3-carbonitrile

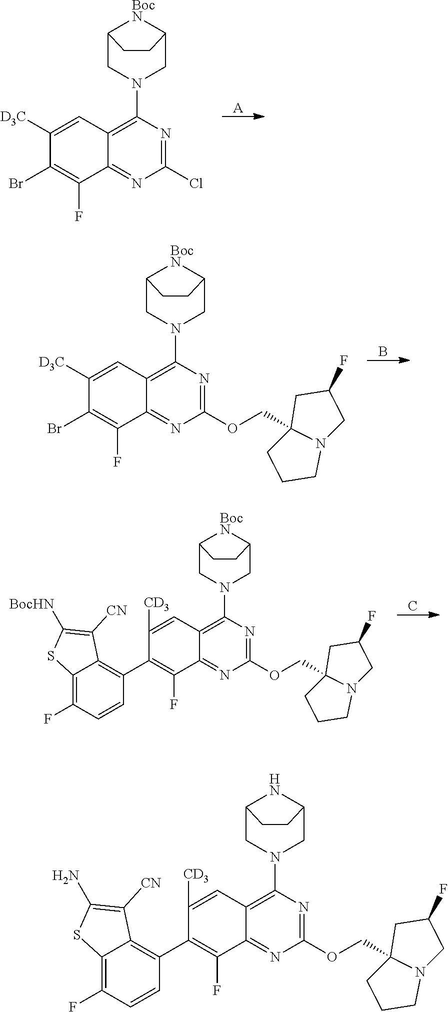

Step A. tert-butyl (1R,5S)-3-(7-bromo-8-fluoro-2-(((2R,7aS)-2-fluorotetrahydro-1H-pyrrolizin-7a(5H)-yl)methoxy)-6-(methyl-d3)quinazolin-4-yl)-3,8-diazabicyclo[3.2.1]octane-8-carboxylate: To a solution of tert-butyl (1R,5S)-3-(7-bromo-2-chloro-8-fluoro-6-(methyl-d3)quinazolin-4-yl)-3,8-diazabicyclo[3.2.1]octane-8-carboxylate (85.0 mg, 1.0 equiv) and ((2R,7aS)-2-fluorotetrahydro-1H-pyrrolizin-7a(5H)-yl)methanol (41.5 mg, 1.5 equiv) in DMF (0.5 mL) and THE (0.5 mL) were added Cs₂CO₃ (170 mg, 3.0 equiv) and 1,4-diazabicyclo[2.2.2]octane (19.5 mg, 1.0 equiv). The reaction was stirred at 25° C. for 1 hour. The mixture was filtered and purified by reversed phase flash chromatography [C18, 0.1% formic acid condition] to afford the title compound (80.0 mg, 74% yield) as yellow solid; LCMS (ESI, M+1, M+3): m/z=611.5, 613.5.

Step B. tert-butyl (1R,5S)-3-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(methyl-d3)quinazolin-4-yl)-3,8-diazabicyclo[3.2.1]octane-8-carboxylate: To a mixture of tert-butyl (1R,5S)-3-(7-bromo-8-fluoro-2-(((2R,7aS)-2-fluorotetrahydro-1H-pyrrolizin-7a(5H)-yl)methoxy)-6-(methyl-d3)quinazolin-4-yl)-3,8-diazabicyclo[3.2.1]octane-8-carboxylate (75.0 mg, 1.0 equiv) and tert-butyl (3-cyano-4-(5,5-dimethyl-1,3,2-dioxaborinan-2-yl)-7-fluorobenzo[b]thiophen-2-yl)carbamate (64.4 mg, 1.3 equiv) in toluene (0.5 mL) were added Pd(DPEPhos)Cl₂ (8.43 mg, 0.1 equiv) and Cs₂CO₃ (120 mg, 3.0 equiv). The reaction was degassed and purged with nitrogen 3 times. The reaction was stirred at 110° C. for 2 hours. The mixture was concentrated and purified by reversed phase flash chromatography [C18, 0.1% formic acid condition] to afford the title compound (50.0 mg, 47% yield) as yellow solid; LCMS (ESI, M+1): m/z=823.6.

Step C. 4-(4-(3,8-diazabicyclo[3.2.1]octan-3-yl)-8-fluoro-2-(((2R,7aS)-2-fluorotetrahydro-1H-pyrrolizin-7a(5H)-yl)methoxy)-6-(methyl-d3)quinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-(methyl-d3)quinazolin-4-yl)-3,8-diazabicyclo[3.2.1]octane-8-carboxylate (45.0 mg, 1.0 equiv) in DCM (0.4 mL) was added TFA (0.2 mL, 49 equiv). The reaction was stirred at 25° C. for 1 hour. The mixture were concentrated and purified by prep-HPLC [Phenomenex luna C18 150×25 mm×10 μm; A: water(0.1% FA), B: ACN, B %: 4%-34% B over 10 min] to afford the title compound (27.4 mg, 74% yield, 0.44 HCOOH) as white solid; ¹H NMR (400 MHz, DMSO-d₆) δ=8.02 (br s, 2H), 7.52 (s, 1H), 7.25-7.16 (m, 1H), 7.14-7.06 (m, 1H), 5.44-5.20 (m, 1H), 4.41 (br d, J=12.4 Hz, 2H), 4.21-4.04 (m, 2H), 4.00 (br s, 2H), 3.60 (br d, J=12.4 Hz, 2H), 3.20-3.05 (m, 4H), 2.95-2.82 (m, 1H), 2.22-1.99 (m, 3H), 1.93-1.71 (m, 7H); LCMS (ESI, M+1): m/z=623.5.

### Example 155

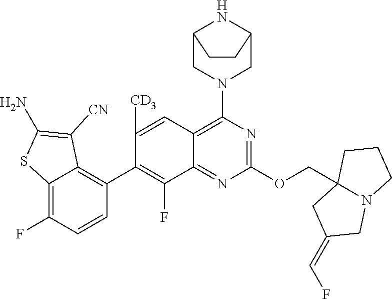 [SMILES: [2H]C([2H])([2H])c1cc2c(N3CC4CCC(C3)N4)nc(OCC34CCCN3C/C(=C\F)C4)nc2c(F)c1-c1ccc(F)c2sc(N)c(C#N)c12]

### 4-(4-((1R,5S)-3,8-diazabicyclo[3.2.1]octan-3-yl)-8-fluoro-2-(((Z)-2-(fluoromethylene)tetrahydro-1H-pyrrolizin-7a(5H)-yl)methoxy)-6-(methyl-d3)quinazolin-7-yl)-2-amino-7-fluorobenzo[b]thiophene-3-carbonitrile

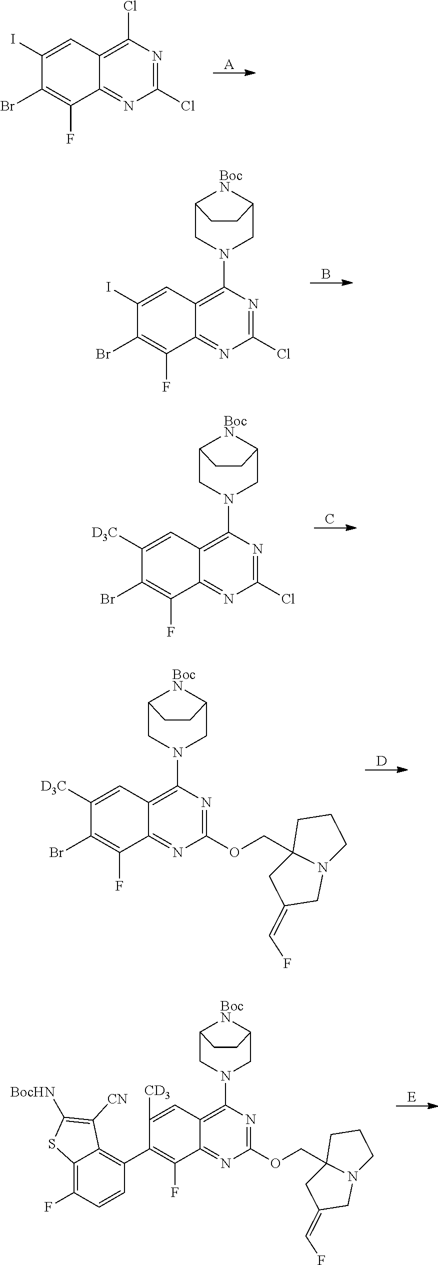

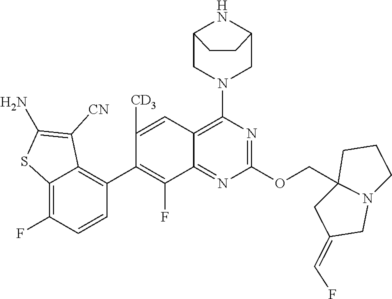 [SMILES: [2H]C([2H])([2H])c1cc2c(N3CC4CCC(C3)N4)nc(OCC34CCCN3C/C(=C\F)C4)nc2c(F)c1-c1ccc(F)c2sc(N)c(C#N)c12]

Step A. tert-butyl (1R,5S)-3-(7-bromo-2-chloro-8-fluoro-6-iodoquinazolin-4-yl)-3,8-diazabicyclo[3.2.1]octane-8-carboxylate: To a solution of 7-bromo-2,4-dichloro-8-fluoro-6-iodoquinazoline (4.40 g, 1.0 equiv) and DIEA (14.8 g, 22 equiv) in DCM (45 mL) was added tert-butyl (1R,5S)-3,8-diazabicyclo[3.2.1]octane-8-carboxylate (1.66 g, 1.5 equiv) at −40° C. The reaction was stirred at −40° C. for 1 hour. The mixture was diluted with water (80.0 mL) and extracted with DCM (3×50 mL). The combined organic layers were washed with brine (2×100 mL), dried over sodium sulfate, concentrated, and purified by column chromatography (SiO₂, Petroleum ether/Ethyl acetate=100/1 to 4/1) to afford the title compound (2.20 g, 68% yield) as white solid; LCMS (ESI, M+1, M+3): m/z=597.0, 598.9.

Step B. tert-butyl (1R,5S)-3-(7-bromo-2-chloro-8-fluoro-6-(methyl-d3)quinazolin-4-yl)-3,8-diazabicyclo[3.2.1]octane-8-carboxylate: To a solution of tert-butyl 3-(7-bromo-2-chloro-8-fluoro-6-iodo-quinazolin-4-yl)-3,8-diazabicyclo[3.2.1]octane-8-carboxylate (300 mg, 1.0 equiv) in THE (6.0 mL) was added n-BuLi (2.5 M, 241 μL, 1.2 equiv) at −78° C. The reaction was stirred at −78° C. for 0.5 hours and iodomethane-d3 (109 mg, 1.5 equiv) was added. The reaction was stirred at 15° C. for 0.5 hours. The mixture was diluted with water (8.0 mL) and extracted with EtOAc (3×4.0 mL). The combined organic layers were dried over sodium sulfate, concentrated, and purified by reversed phase flash chromatography [C18, 0.1% formic acid condition] to afford the title compound (90.0 mg, 36% yield) as yellow solid; LCMS (ESI, M+1, M+3): m/z=488.2, 490.2.

Step C. tert-butyl (1R,5S)-3-(7-bromo-8-fluoro-2-(((S,Z)-2-(fluoromethylene)tetrahydro-1H-pyrrolizin-7a(5H)-yl)methoxy)-6-(methyl-d3)quinazolin-4-yl)-3,8-diazabicyclo[3.2.1]octane-8-carboxylate: To a solution of tert-butyl (1R,5S)-3-(7-bromo-2-chloro-8-fluoro-6-(methyl-d3)quinazolin-4-yl)-3,8-diazabicyclo[3.2.1]octane-8-carboxylate (140 mg, 1.0 equiv) and (S,Z)-(2-(fluoromethylene)tetrahydro-1H-pyrrolizin-7a(5H)-yl)methanol (54.0 mg, 1.1 equiv) in DMF (0.5 mL) and THE (0.5 mL) were added Cs₂CO₃ (280 mg, 3.0 equiv) and 1,4-diazabicyclo[2.2.2]octane (32.1 mg, 1.0 equiv). The reaction was stirred at 25° C. for 1 hour. The mixture was filtered and purified by reversed phase flash chromatography [C18, 0.1% formic acid condition] to afford the title compound (35.0 mg, 16% yield) as yellow solid; LCMS (ESI, M+1, M+3): m/z=623.4, 625.4.

Step D. tert-butyl (1R,5S)-3-(7-(2-((tert-butoxycarbonyl)amino)-3-cyano-7-fluorobenzo[b]thiophen-4-yl)-8-fluoro-2-(((S,Z)-2-(fluoromethylene)tetrahydro-1H-pyrrolizin-7a(5H)-yl)methoxy)-6-(methyl-d3)quinazolin-4-yl)-3,8-diazabicyclo[3.2.1]octane-8-carboxylate: To a mixture of tert-butyl (1R,5S)-3-(7-bromo-8-fluoro-2-(((S,Z)-2-(fluoromethylene)tetrahydro-1H-pyrrolizin-7a(5H)-yl)methoxy)-6-(methyl-d3)quinazolin-4-yl)-3,8-diazabicyclo[3.2.1]octane-8-carboxylate (60.0 mg, 1.0 equiv) and tert-butyl (3-cyano-4-(5,5-dimethyl-1,3,2-dioxaborinan-2-yl)-7-fluorobenzo[b]thiophen-2-yl)carbamate (43.0 mg, 1.3 equiv) in toluene (0.5 mL) were added Pd(DPEPhos)Cl₂ (5.63 mg, 0.1 equiv) and Cs₂CO₃ (80.0 mg, 3.0 equiv). The reaction was degassed and purged with nitrogen 3 times. The reaction was stirred at 110° C. for 2 hours. The mixture was concentrated and purified by reversed phase flash chromatography [C18, 0.1% formic acid condition] to afford the title compound (50.0 mg, 47% yield) as yellow solid; LCMS (ESI, M+1): m/z=835.5.

Step E. 4-(4-((1R,5S)-3,8-diazabicyclo[3.2.1]octan-3-yl)-8-fluoro-2-(((Z)-2-(fluoromethylene)tetrahydro-1H-pyrrolizin-7a(5H)-yl)methoxy)-6-(methyl-d3)quinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-8-fluoro-2-(((S,Z)-2-(fluoromethylene)tetrahydro-1H-pyrrolizin-7a(5H)-yl)methoxy)-6-(methyl-d3)quinazolin-4-yl)-3,8-diazabicyclo[3.2.1]octane-8-carboxylate (45.0 mg, 1.0 equiv) in DCM (0.4 mL) was added TFA (307 mg, 49 equiv). The reaction was stirred at 25° C. for 1 hour. The mixture was concentrated and then saturated NaHCO₃ aqueous solution (1.0 mL) was added. The mixture was extracted with EtOAc (3×2.0 mL). The combined organic layers were washed with brine (3×2.0 mL), dried over anhydrous sodium sulfate, concentrated and purified by prep-HPLC [Phenomenex luna C18 150×25 mm×10 μm; A: water(0.1% FA), B: ACN, B %: 9%-39% B over 10 min] to afford the title compound (21.9 mg, 63% yield) as white solid; ¹H NMR (400 MHz, DMSO-d₆) S=8.21-7.97 (m, 2H), 7.50 (s, 1H), 7.24-7.14 (m, 1H), 7.13-7.02 (m, 1H), 6.89-6.58 (m, 1H), 4.42-4.25 (m, 2H), 4.14-3.98 (m, 2H), 3.80-3.66 (m, 3H), 3.53-3.43 (m, 3H), 3.06-2.96 (m, 1H), 2.70-2.56 (m, 2H), 2.34 (br d, J=9.6 Hz, 2H), 2.03-1.59 (m, 8H); LCMS (ESI, M+1): m/z=635.4.

### Example 156

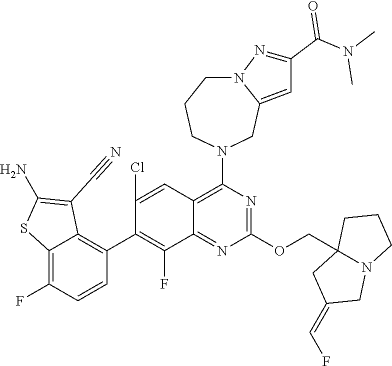 [SMILES: CN(C)C(=O)c1cc2n(n1)CCCN(c1nc(OCC34CCCN3C/C(=C\F)C4)nc3c(F)c(-c4ccc(F)c5sc(N)c(C#N)c45)c(Cl)cc13)C2]

### (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

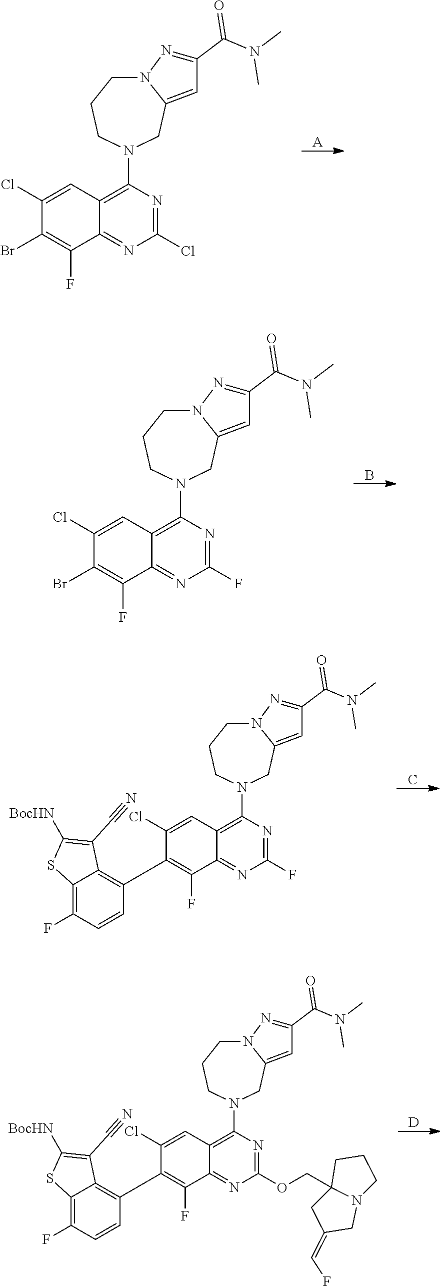

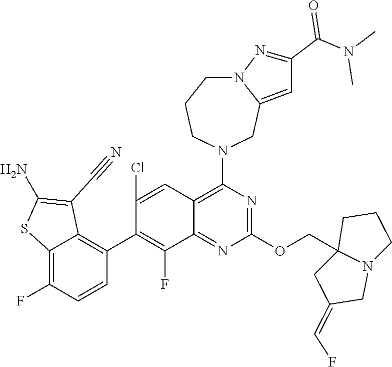 [SMILES: CN(C)C(=O)c1cc2n(n1)CCCN(c1nc(OCC34CCCN3C/C(=C\F)C4)nc3c(F)c(-c4ccc(F)c5sc(N)c(C#N)c45)c(Cl)cc13)C2]

Step A. 5-(7-bromo-6-chloro-2,8-difluoroquinazolin-4-yl)-N,N-dimethyl-5,6,7,8-tetrahydro-4H-pyrazolo[1,5-a][1,4]diazepine-2-carboxamide: To a solution of 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide (600 mg, 1.0 equiv) in DMSO (6 mL) was added KF (2.08 g, 30 equiv). The reaction was stirred at 140° C. for 0.5 hours. The mixture was diluted with water (10 mL) and extracted with EtOAc (2×10 mL). The combined organic layers were washed with brine (2×10 mL), dried over anhydrous sodium sulfate, filtered, and concentrated to afford the title compound (580 mg, 93% yield) as yellow solid; LCMS (ESI, M+1, M+3): m/z=485.2, 487.2.

Step B. tert-butyl (4-(6-chloro-4-(2-(dimethylcarbamoyl)-7,8-dihydro-4H-pyrazolo[1,5-a][1,4]diazepin-5(6H)-yl)-2,8-difluoroquinazolin-7-yl)-3-cyano-7-fluorobenzo[b]thiophen-2-yl)carbamate: To a solution of 5-(7-bromo-6-chloro-2,8-difluoro-quinazolin-4-yl)-N,N-dimethyl-4,6,7,8-tetrahydropyrazolo[1,5-a][1,4]diazepine-2-carboxamide (300 mg, 1.0 equiv) and tert-butyl (3-cyano-4-(5,5-dimethyl-1,3,2-dioxaborinan-2-yl)-7-fluorobenzo[b]thiophen-2-yl)carbamate (624 mg, 2.5 equiv) in dioxane (3 mL) were added dichloro[bis(diphenylphosphinophenyl)ether]palladium(II) (84.9 mg, 0.2 equiv) and Cs₂CO₃ (604 mg, 3.0 equiv). The reaction was stirred at 60° C. for 1 hour. The mixture was diluted with water (10 mL) and extracted with EtOAc (2×10 mL). The combined organic layers were washed with brine (2×10 mL), dried over anhydrous sodium sulfate, concentrated, and purified by reversed phase flash chromatography [C18, 0.1% formic acid condition] to afford the title compound (300 mg, 70% yield) as red solid; LCMS (ESI, M+1): m/z=697.1.

Step C. tert-butyl (Z)-(4-(6-chloro-4-(2-(dimethylcarbamoyl)-7,8-dihydro-4H-pyrazolo[1,5-a][1,4]diazepin-5(6H)-yl)-8-fluoro-2-((2-(fluoromethylene)tetrahydro-1H-pyrrolizin-7a(5H)-yl)methoxy)guinazolin-7-yl)-3-cyano-7-fluorobenzo[b]thiophen-2-yl)carbamate: To a solution of (Z)-(2-(fluoromethylene)tetrahydro-1H-pyrrolizin-7a(5H)-yl)methanol (73.7 mg, 1.0 equiv) in THE (1.5 mL) was added NaH (51.6 mg, 60% purity, 3.0 equiv). The reaction was stirred at 0° C. for 0.5 hour and a solution of tert-butyl N-[4-[6-chloro-4-[2-(dimethylcarbamoyl)-4,6,7,8-tetrahydropyrazolo[1,5-a][1,4]diazepin-5-yl]-2,8-difluoro-quinazolin-7-yl]-3-cyano-7-fluoro-benzothiophen-2-yl]carbamate (300 mg, 1.0 equiv) in THE (1.5 mL) was added dropwise at 25° C. The reaction was stirred at 25° C. for 1 hour. The mixture was diluted with water (10 mL) and extracted with EtOAc (2×10 mL). The combined organic layers were washed with brine (2×10 mL), dried over anhydrous sodium sulfate, concentrated, and purified by reversed phase flash chromatography [C18, 0.1% formic acid condition] to afford the title compound (35 mg, 9.6% yield) as red solid; LCMS (ESI, M+1): m/z=848.2.

Step D. (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)guinazolin-4-yl)-N,N-dimethyl-5,6,7,8-tetrahydro-4H-pyrazolo[1,5-a][1,4]diazepine-2-carboxamide: To a solution of tert-butyl (Z)-(4-(6-chloro-4-(2-(dimethylcarbamoyl)-7,8-dihydro-4H-pyrazolo[1,5-a][1,4]diazepin-5(6H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate (30 mg, 1.0 equiv) in DCM (0.5 mL) was added TFA (4.61 g, 1142 equiv). The reaction was stirred at 25° C. for 0.5 hour. The mixture was concentrated. The residue was dissolved in water (1 mL), neutralized with solid NaHCO₃ and extracted with EtOAc (2×2 mL). The combined organic layers were dried over anhydrous sodium sulfate, concentrated and purified by prep-HPLC [Phenomenex luna C18 150×25 mm×10 μm; A: water(FA), B: ACN; B %: 25%-55% over 9 min] to afford the title compound (13.8 mg, 51% yield) as white solid; ¹H NMR (400 MHz, METHANOL-d₄) δ=8.01 (d, J=1.6 Hz, 1H), 7.20 (ddd, J=0.8, 5.2, 8.4 Hz, 1H), 7.02 (dd, J=8.4, 9.2 Hz, 1H), 6.84-6.56 (m, 2H), 5.08 (s, 2H), 4.55-4.47 (m, 2H), 4.43-4.27 (m, 4H), 4.11-3.99 (m, 1H), 3.77-3.64 (m, 1H), 3.38 (br s, 1H), 3.32 (s, 3H), 3.06 (s, 3H), 2.96-2.75 (m, 2H), 2.59-2.50 (m, 1H), 2.43-2.32 (m, 2H), 2.24-2.13 (m, 1H), 2.11-1.89 (m, 3H); LCMS (ESI, M+1): m/z=748.4.

### Example 157

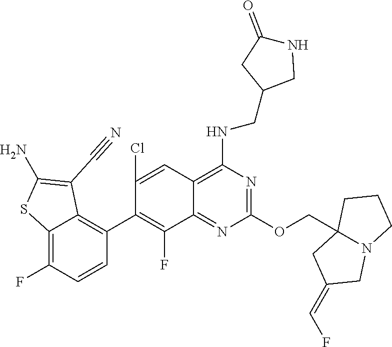 [SMILES: N#Cc1c(N)sc2c(F)ccc(-c3c(Cl)cc4c(NCC5CNC(=O)C5)nc(OCC56CCCN5C/C(=C\F)C6)nc4c3F)c12]

### (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

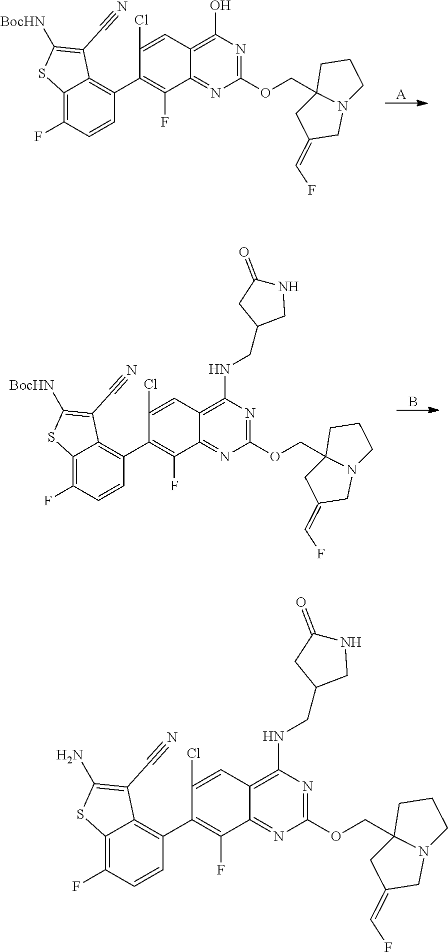

Step A. tert-butyl (Z)-(4-(6-chloro-8-fluoro-2-((2-(fluoromethylene)tetrahydro-1H-pyrrolizin-7a(5H)-yl)methoxy)-4-(((5-oxopyrrolidin-3-yl)methyl)amino)quinazolin-7-yl)-3-cyano-7-fluorobenzo[b]thiophen-2-yl)carbamate: To a solution of tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) and TEA (46.1 mg, 3.0 equiv) in DMSO (1.0 mL) was added PyBOP (119 mg, 1.5 equiv). The reaction was stirred at 25° C. for 20 minutes and added 4-(aminomethyl)pyrrolidin-2-one (34.3 mg, 1.5 equiv, HCl) was added. The reaction was stirred at 25° C. for 2 hours. The mixture was filtered and purified with reversed phase flash chromatography [C18, 0.1% formic acid condition] to afford the title compound (42.0 mg, 32% yield) as yellow solid; LCMS (ESI, M+1): m/z=754.2.

Step B. (Z)-2-amino-4-(6-chloro-8-fluoro-2-((2-(fluoromethylene)tetrahydro-1H-pyrrolizin-7a(5H)-yl)methoxy)-4-(((5-oxopyrrolidin-3-yl)methyl)amino)quinazolin-7-yl)-7-fluorobenzo[b]thiophene-3-carbonitrile: To a solution of tert-butyl (Z)-(4-(6-chloro-8-fluoro-2-((2-(fluoromethylene)tetrahydro-1H-pyrrolizin-7a(5H)-yl)methoxy)-4-(((5-oxopyrrolidin-3-yl)methyl)amino)quinazolin-7-yl)-3-cyano-7-fluorobenzo[b]thiophen-2-yl)carbamate (50.0 mg, 1.0 equiv) in DCM (1.5 mL) was added TFA (2.30 g, 305 equiv). The reaction was stirred at 20° C. for 0.5 hours. The mixture was concentrated, neutralized with saturated aqueous NaHCO₃(1.0 mL) and extracted with ethyl acetate (3×1 mL). Combined the organic layers were dried over Na₂SO₄, concentrated in vacuum and purified by prep-HPLC [column: Phenomenex luna C18 150×25 mm×10 μm; A: water (FA)−ACN, B: ACN, B %: 20%-50% over 9 min] to afford the title compound (24.6 mg, 56% yield, 0.1 HCOOH) as white solid; ¹H NMR (400 MHz, METHANOL-d₄) δ=8.12 (s, 1H), 7.24-7.16 (m, 1H), 7.04 (t, J=8.8 Hz, 1H), 6.86-6.59 (m, 1H), 4.53-4.34 (m, 2H), 4.18-4.02 (m, 1H), 3.80-3.67 (m, 3H), 3.58 (dd, J=7.6, 10.0 Hz, 1H), 3.47-3.37 (m, 1H), 3.27 (dd, J=5.2, 10.4 Hz, 1H), 3.07-2.90 (m, 2H), 2.85 (br d, J=15.6 Hz, 1H), 2.48-2.62 (m, 2H), 2.29-2.18 (m, 2H), 2.15-1.91 (m, 3H); LCMS (ESI, M+1): m/z=654.2.

### Example 158

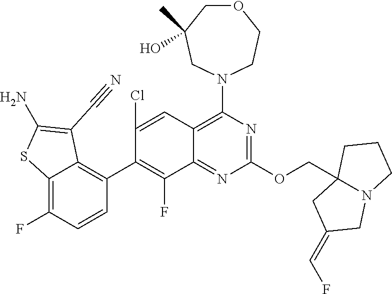 [SMILES: C[C@@]1(O)COCCN(c2nc(OCC34CCCN3C/C(=C\F)C4)nc3c(F)c(-c4ccc(F)c5sc(N)c(C#N)c45)c(Cl)cc23)C1]

### 2-amino-4-(6-chloro-8-fluoro-2-(((Z)-2-(fluoromethylene)tetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile

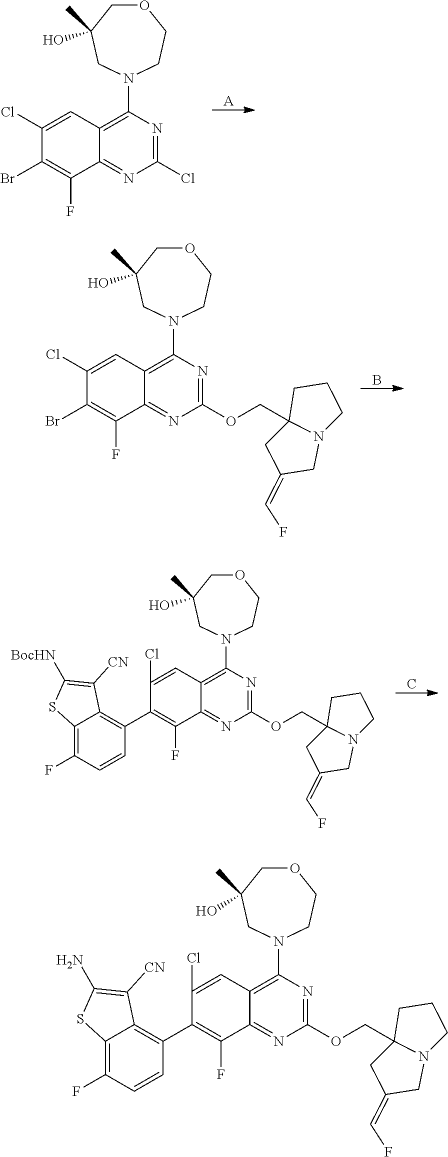

Step A. (6S)-4-(7-bromo-6-chloro-8-fluoro-2-(((Z)-2-(fluoromethylene)tetrahydro-1H-pyrrolizin-7a(5H)-yl)methoxy)quinazolin-4-yl)-6-methyl-1,4-oxazepan-6-ol: To a mixture of (S)-4-(7-bromo-2,6-dichloro-8-fluoroquinazolin-4-yl)-6-methyl-1,4-oxazepan-6-ol (200 mg, 1.0 equiv), Cs₂CO₃ (460 mg, 3.0 equiv), and DABCO (52.8 mg, 1.0 equiv) in DMF (2.5 mL) and THF (2.5 mL) was added (Z)-(2-(fluoromethylene)tetrahydro-1H-pyrrolizin-7a(5H)-yl)methanol (96.7 mg, 1.2 equiv). The reaction was stirred at 30° C. for 12 hours. The mixture was diluted with water (5.0 mL) and extracted with ethyl acetate (3×5 mL). The combined organic layers were dried over anh Na₂SO₄, concentrated and purified by silica gel chromatography (petroleum ether/ethyl acetate 30/1 to ethyl acetate/methanol 20/1), followed by reversed phase flash chromatography [C18, 0.1% formic acid condition] to afford the title compound (70 mg, 26% yield) as yellow solid; LCMS (ESI, M+1, M+3): m/z=558.9, 560.9.

Step B. tert-butyl (4-(6-chloro-8-fluoro-2-(((Z)-2-(fluoromethylene)tetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate: To a mixture of (6S)-4-(7-bromo-6-chloro-8-fluoro-2-(((Z)-2-(fluoromethylene)tetrahydro-1H-pyrrolizin-7a(5H)-yl)methoxy)quinazolin-4-yl)-6-methyl-1,4-oxazepan-6-ol (10 mg, 1.0 equiv), tert-butyl N-[3-cyano-4-(5,5-dimethyl-1,3,2-dioxaborinan-2-yl)-7-fluoro-benzothiophen-2-yl]carbamate (10.9 mg, 1.5 equiv) and Cs₂CO₃ (17.5 mg, 3.0 equiv) in toluene (0.4 mL) was added PdCl₂(DPEPhos) (2.46 mg, 0.2 equiv). The reaction was degassed and purged with N₂ for 3 times. The reaction was stirred at 110° C. for 0.5 hours. The mixture was diluted with water (3.0 mL) and extracted with ethyl acetate (3×3.0 mL). The combined organic layers were dried over anhydrous Na₂SO₄, concentrated, and purified with prep-TLC (SiO₂, dichloromethane: methanol=10:1) to afford the title compound (20 mg, crude) as yellow solid.

Step C. 2-amino-4-(6-chloro-8-fluoro-2-(((Z)-2-(fluoromethylene)tetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: To a solution of tert-butyl (4-(6-chloro-8-fluoro-2-(((Z)-2-(fluoromethylene)tetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate (20 mg, 1.0 equiv) in DCM (0.5 mL) was added TFA (767 mg, 259 equiv). The reaction was stirred at 20° C. for 0.5 hours. The mixture was concentrated, basified with saturated aqueous NaHCO₃(1.0 mL) and extracted with ethyl acetate (3×1 mL). The combined organic layers were dried over Na₂SO₄, concentrated and purified by prep-HPLC [column: Phenomenex luna C18 150×25 mm×10 um; A: water (FA), B: ACN, B %: 18%-48% B over 10 min] and lyophilized to afford the title compound (2.73 mg, 15% yield, 0.13 HCOOH) as off-white solid; ¹H NMR (400 MHz, METHANOL-d₄) δ=8.46-8.36 (m, 1H), 7.26-7.17 (m, 1H), 7.04 (t, J=9.2 Hz, 1H), 6.81-6.52 (m, 1H), 4.48-4.27 (m, 4H), 4.11-3.97 (m, 2H), 3.96-3.80 (m, 3H), 3.71-3.62 (m, 2H), 3.62-3.52 (m, 1H), 3.28-3.18 (m, 1H), 2.85-2.70 (m, 2H), 2.54-2.43 (m, 1H), 2.20-2.11 (m, 1H), 2.07-1.84 (m, 3H), 1.26 (s, 3H); LCMS (ESI, M+1): m/z=671.2.

### Example 159

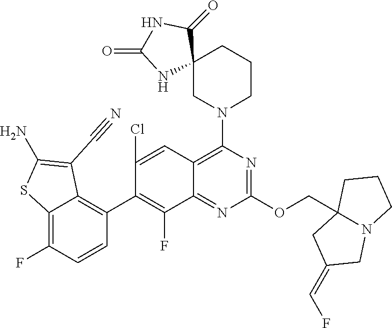 [SMILES: N#Cc1c(N)sc2c(F)ccc(-c3c(Cl)cc4c(N5CCC[C@]6(C5)NC(=O)NC6=O)nc(OCC56CCCN5C/C(=C\F)C6)nc4c3F)c12]

### 2-amino-4-(6-chloro-4-((R)-2,4-dioxo-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-7-fluorobenzo[b]thiophene-3-carbonitrile

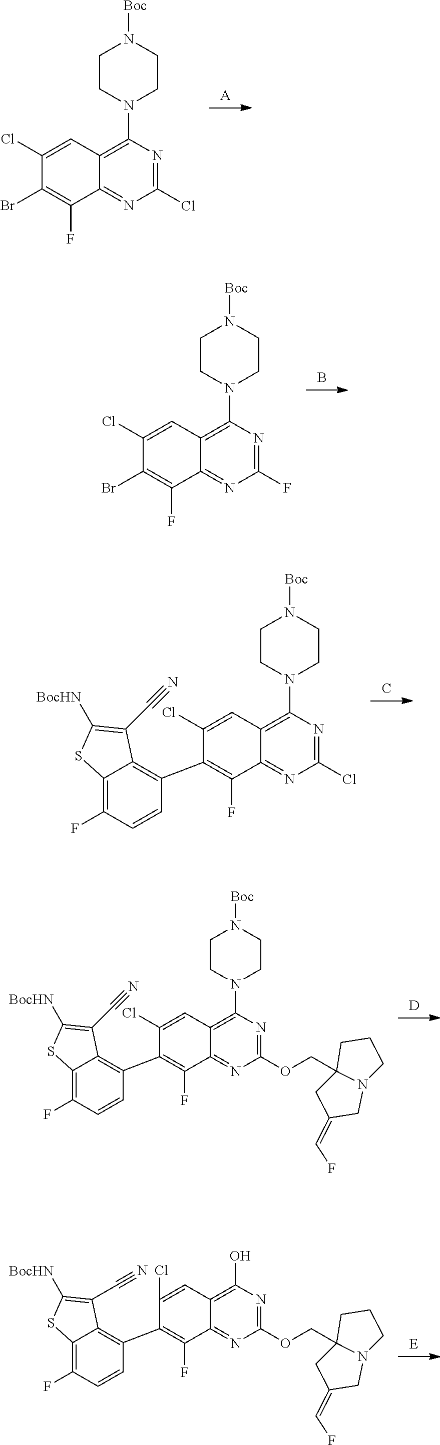

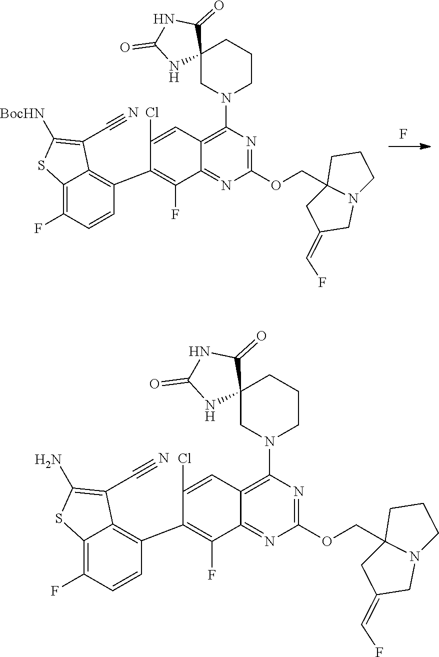

Step A. tert-butyl 4-(7-bromo-6-chloro-2,8-difluoroquinazolin-4-yl)piperazine-1-carboxylate: To a solution of tert-butyl 4-(7-bromo-2,6-dichloro-8-fluoroquinazolin-4-yl)piperazine-1-carboxylate (3.00 g, 1.0 equiv) in DMSO (20.0 mL) were added KF (3.63 g, 10 equiv) and 1,4,7,10,13,16-hexaoxacyclooctadecane (165 mg, 0.1 equiv). The reaction was stirred at 95° C. for 3 hours and at 120° C. for 1 hour. The mixture was diluted with water (100 mL) and extracted with ethyl acetate (2×50 mL). The combined organic layers were dried over anhydrous sodium sulfate and concentrated to afford the title compound (2.89 g, crude) as yellow solid.

Step B. tert-butyl 4-(7-(2-((tert-butoxycarbonyl)amino)-3-cyano-7-fluorobenzo[b]thiophen-4-yl)-6-chloro-2,8-difluoroquinazolin-4-yl)piperazine-1-carboxylate: To a mixture of tert-butyl 4-(7-bromo-6-chloro-2,8-difluoroquinazolin-4-yl)piperazine-1-carboxylate (1.00 g, 1.0 equiv) and tert-butyl (3-cyano-4-(5,5-dimethyl-1,3,2-dioxaborinan-2-yl)-7-fluorobenzo[b]thiophen-2-yl)carbamate (1.31 g, 1.5 equiv) in THE (15.0 mL) was added Cs₂CO₃ (2.11 g, 3.0 equiv) and PdCl₂(DPEPhos) (222 mg, 0.15 equiv). The reaction was degassed and purged with N₂ for 3 times. The reaction was stirred at 60° C. for 3 hours. The mixture was diluted with water (100 mL) and extracted with ethyl acetate (2×50 mL). The combined organic phase was washed with brine (30 mL), dried over anhydrous sodium sulfate, concentrated, and purified with reversed phase flash chromatography [C18, 0.1% formic acid condition] to afford the title compound (1.23 g, 81% yield) as yellow solid; LCMS (ESI, M+1): m/z=675.2.

Step C. tert-butyl (Z)-4-(7-(2-((tert-butoxycarbonyl)amino)-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)piperazine-1-carboxylate: To a solution of (Z)-(2-(fluoromethylene)tetrahydro-1H-pyrrolizin-7a(5H)-yl)methanol (304 mg, 1.0 equiv) in THE (10 mL) was added NaH (213 mg, 60% purity, 3.0 equiv) at 0° C. The reaction was stirred at 0° C. for 0.5 hours and a solution of tert-butyl 4-(7-(2-((tert-butoxycarbonyl)amino)-3-cyano-7-fluorobenzo[b]thiophen-4-yl)-6-chloro-2,8-difluoroquinazolin-4-yl)piperazine-1-carboxylate (1.20 g, 1.0 equiv) in THE (10.0 mL) was added dropwise. The reaction was stirred at 10° C. for 0.5 hours. The mixture was quenched with water (50 mL) and extracted with ethyl acetate (2×25 mL). The combined organic layers were dried over anhydrous sodium sulfate, filtered, and concentrated to afford the title compound (1.54 g, crude) as yellow solid; LCMS (ESI, M+1): m/z=826.4.

Step D. tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate: To a solution of tert-butyl (Z)-4-(7-(2-((tert-butoxycarbonyl)amino)-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)piperazine-1-carboxylate (1.54 g, 1.0 equiv) in EtOH (50 mL) and H₂O (5 mL) was added NaOH (373 mg, 5.0 equiv). The reaction was stirred at 50° C. for 16 hours. The mixture was diluted with water (20 mL) and concentrated to remove EtOH. The pH of the mixture was adjusted 6 with saturated citric acid solution (20 mL) and extracted with ethyl acetate (3×25 mL). The combined organic layers were dried over anhydrous sodium sulfate, concentrated, and purified with reversed phase flash chromatography [C18, 0.1% formic acid condition] to afford the title compound (800 mg, 58% yield) as yellow solid; LCMS (ESI, M+1): m/z=658.1.

Step E. tert-butyl (4-(6-chloro-4-((R)-2,4-dioxo-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (20 mg, 1.0 equiv) and TEA (9.23 mg, 3.0 equiv) in DMSO (0.20 mL) was added PyBOP (23.7 mg, 1.5 equiv). The reaction was stirred at 25° C. for 0.5 hours and (R)-1,3,7-triazaspiro[4.5]decane-2,4-dione (10.3 mg, 2.0 equiv) was added. The reaction was stirred at 45° C. for 6 hours. The mixture was diluted with water (10 mL) and extracted with ethyl acetate (2×5 mL). The combined organic layers were dried over anhydrous sodium sulfate, concentrated to afford the title compound (60 mg, crude) as yellow solid; LCMS (ESI, M+1): m/z=831.2.

Step F. 2-amino-4-(6-chloro-4-((R)-2,4-dioxo-1,3,7-triazaspiro[4.5]decan-7-yl)-8-fluoro-2-(((Z)-2-(fluoromethylene)tetrahydro-1H-pyrrolizin-7a(5H)-yl)methoxy)guinazolin-7-yl)-7-fluorobenzo[b]thiophene-3-carbonitrile: To a solution of tert-butyl (4-(6-chloro-4-((R)-2,4-dioxo-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (60.0 mg, 1.0 equiv) in DCM (1.00 mL) was added TFA (1.54 g, 1.00 mL). The reaction was stirred at 10° C. for 20 min. The mixture was concentrated, neutralized with saturated NaHCO₃ solution (20 mL) and extracted with ethyl acetate (2×10 mL). The combined organic layers were dried over anhydrous sodium sulfate, concentrated and purified by prep-HPLC [column: Waters Xbridge 150×25 mm×5 μm; A: water (NH₄HCO₃), B: ACN; B %: 42%-72% over 9 min] to afford the title compound (4.44 mg, 80% yield) as orange solid; 1H NMR (400 MHz, METHANOL-d₄) δ=7.97 (s, 1H), 7.25-7.18 (m, 1H), 7.04 (t, J=8.8 Hz, 1H), 6.77-6.49 (d, J=8.8 Hz, 1H), 4.42-4.19 (m, 4H), 3.85 (d, J=14.4 Hz, 1H), 3.62-3.54 (m, 1H), 3.52-3.41 (d, J=14.4 Hz, 2H), 3.12 (m, 1H), 2.78-2.66 (m, 2H), 2.43 (d, J=16.0 Hz, 1H), 2.22-1.82 (m, 8H) LCMS (ESI, M+1): m/z=709.2.

### Example 160

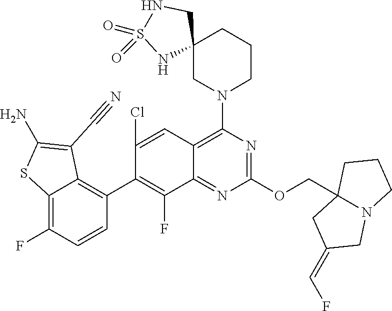 [SMILES: N#Cc1c(N)sc2c(F)ccc(-c3c(Cl)cc4c(N5CCC[C@]6(CNS(=O)(=O)N6)C5)nc(OCC56CCCN5C/C(=C\F)C6)nc4c3F)c12]

### 2-amino-4-(6-chloro-4-((R)-2,2-dioxido-2-thia-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-7-fluorobenzo[b]thiophene-3-carbonitrile

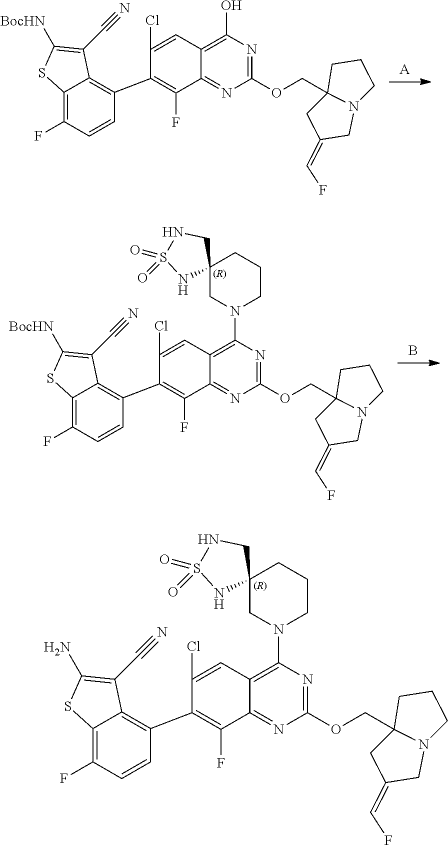

Step A. tert-butyl (4-(6-chloro-4-((R)-2,2-dioxido-2-thia-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (20.0 mg, 1.0 equiv) and TEA (9.23 mg, 3.0 equiv) in DMSO (0.20 mL) was added PyBOP (23.7 mg, 1.5 equiv). The reaction was stirred at 25° C. for 0.5 hours and (R)-2-thia-1,3,7-triazaspiro[4.5]decane 2,2-dioxide (11.6 mg, 2.0 equiv) was added. The reaction was stirred at 45° C. for 6 hours. The mixture was diluted with water (10 mL) and extracted with ethyl acetate (2×5 mL). The combined organic layers were dried over anhydrous sodium sulfate and concentrated to afford the title compound (60 mg, crude) as yellow solid; LCMS (ESI, M+1).

Step B. 2-amino-4-(6-chloro-4-((R)-2,2-dioxido-2-thia-1,3,7-triazaspiro[4.5]decan-7-yl)-8-fluoro-2-(((Z)-2-(fluoromethylene)tetrahydro-1H-pyrrolizin-7a(5H)-yl)methoxy)guinazolin-7-yl)-7-fluorobenzo[b]thiophene-3-carbonitrile: To a solution of tert-butyl (4-(6-chloro-4-((R)-2,2-dioxido-2-thia-1,3,7-triazaspiro[4.5]decan-7-yl)-8-fluoro-2-(((Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (60 mg, 1.0 equiv) in DCM (1.00 mL) was added TFA (1.54 g, 1.00 mL). The reaction was stirred at 10° C. for 20 minutes. The mixture was concentrated under vacuum, neutralized with saturated NaHCO₃ solution (20 mL) and extracted with ethyl acetate (2×10 mL). The combined organic layers were dried over anhydrous sodium sulfate, concentrated and purified by prep-HPLC [column: Waters Xbridge 150×25 mm×5 μm; A: water (NH₄HCO₃), B: ACN; B %: 42%-72% over 9 min] to afford the title compound (6.74 mg, 80% yield) as off-white solid; 1H NMR (400 MHz, METHANOL-d₄) δ=8.00-7.90 (d, J=8.8 Hz, 1H), 7.27-7.17 (m, 1H), 7.10-7.00 (t, J=8.8 Hz, 1H), 6.79-6.50 (d, J=84 Hz,1H), 4.40-4.21 (m, 2H), 4.18-4.02 (m, 1H), 4.01-3.91 (m, 1H), 3.90-3.68 (m, 3H), 3.47 (m, 2H), 3.27-3.11 (m, 2H), 2.80-2.66 (m, 2H), 2.49-2.38 (d, J=1.2 Hz, 1H), 2.20-1.82 (m, 8H); LCMS (ESI, M+1): m/z=731.2.

### Example 161

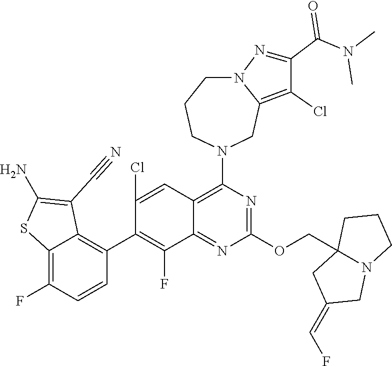 [SMILES: CN(C)C(=O)c1nn2c(c1Cl)CN(c1nc(OCC34CCCN3C/C(=C\F)C4)nc3c(F)c(-c4ccc(F)c5sc(N)c(C#N)c45)c(Cl)cc13)CCC2]

### (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-3-chloro-N,N-dimethyl-5,6,7,8-tetrahydro-4H-pyrazolo[1,5-a][1,4]diazepine-2-carboxamide

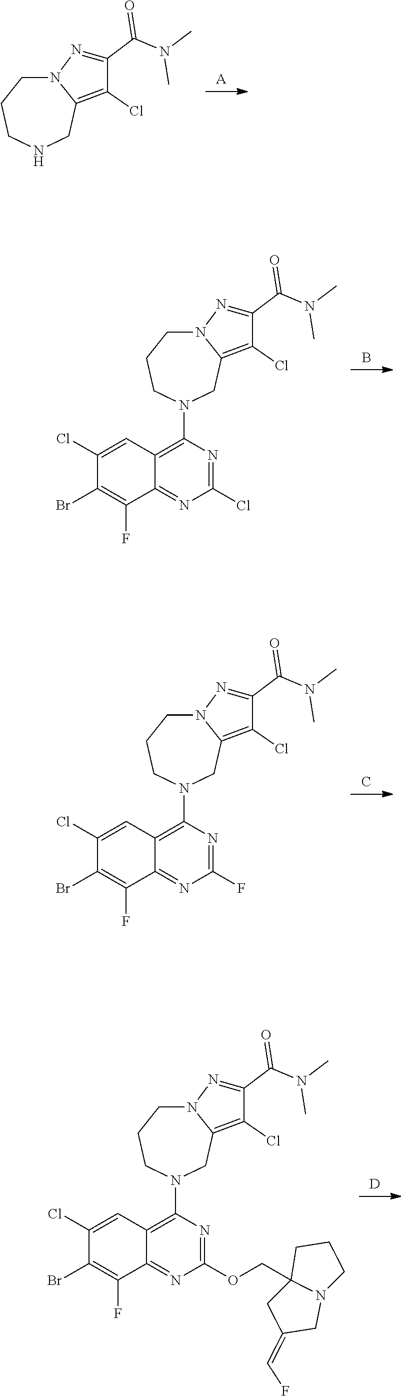

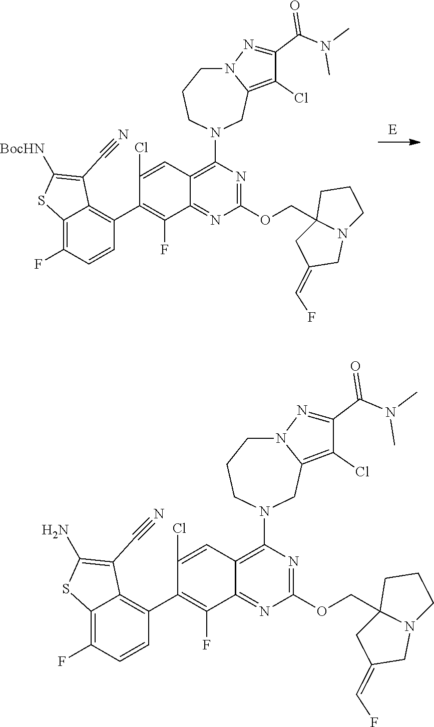 [SMILES: CN(C)C(=O)c1nn2c(c1Cl)CN(c1nc(OCC34CCCN3C/C(=C\F)C4)nc3c(F)c(-c4ccc(F)c5sc(NC(=O)OC(C)(C)C)c(C#N)c45)c(Cl)cc13)CCC2 ; CN(C)C(=O)c1nn2c(c1Cl)CN(c1nc(OCC34CCCN3C/C(=C\F)C4)nc3c(F)c(-c4ccc(F)c5sc(N)c(C#N)c45)c(Cl)cc13)CCC2]

Step A. 5-(7-bromo-2,6-dichloro-8-fluoroquinazolin-4-yl)-3-chloro-N,N-dimethyl-5,6,7,8-tetrahydro-4H-pyrazolo[1,5-a][1,4]diazepine-2-carboxamide: To a solution of 3-chloro-N,N-dimethyl-5,6,7,8-tetrahydro-4H-pyrazolo[1,5-a][1,4]diazepine-2-carboxamide (1.10 g, 1.0 equiv) and 7-bromo-2,4,6-trichloro-8-fluoro-quinazoline (1.50 g, 1.0 equiv) in DCM (20 mL) was added DIEA (2.93 g, 5.0 equiv). The reaction was stirred at −40° C. for 1 hour. The mixture was diluted with water (100 mL) and extracted with ethyl acetate (2×50 mL). The combined organic layers were washed with brine (100 mL), dried over anhydrous sodium sulfate, concentrated and triturated with DCM (10 mL) at 25° C. to afford the title compound (760 mg, 31% yield) as yellow solid; LCMS (ESI, M+1, M+3): m/z=535.0, 537.0.

Step B. 5-(7-bromo-6-chloro-2,8-difluoroquinazolin-4-yl)-3-chloro-N,N-dimethyl-5,6,7,8-tetrahydro-4H-pyrazolo[1,5-a][1,4]diazepine-2-carboxamide: To a solution of 5-(7-bromo-2,6-dichloro-8-fluoro-quinazolin-4-yl)-3-chloro-N,N-dimethyl-4,6,7,8-tetrahydropyrazolo[1,5-a][1,4]diazepine-2-carboxamide (400 mg, 1.0 equiv) in DMSO (4 mL) was added KF (866 mg, 20 equiv). The reaction was stirred at 140° C. for 0.5 hours. The mixture was diluted with water (30 mL) and extracted with DCM (2×20 mL). The combined organic layers were washed with brine (25 mL), dried over anhydrous sodium sulfate, concentrated and triturated with DCM (5 mL) at 25° C. to afford the title compound (280 mg, 71% yield) as yellow solid; LCMS (ESI, M+1, M+3): m/z=519.0, 521.0.

Step C. (Z)-5-(7-bromo-6-chloro-8-fluoro-2-((2-(fluoromethylene)tetrahydro-1H-pyrrolizin-7a(5H)-yl)methoxy)guinazolin-4-yl)-3-chloro-N,N-dimethyl-5,6,7,8-tetrahydro-4H-pyrazolo[1,5-a][1,4]diazepine-2-carboxamide: To a mixture of 5-(7-bromo-6-chloro-2,8-difluoro-quinazolin-4-yl)-3-chloro-N,N-dimethyl-4,6,7,8-tetrahydropyrazolo[1,5-a][1,4]diazepine-2-carboxamide (100 mg, 1.0 equiv) in DMSO (0.6 mL) was added (Z)-(2-(fluoromethylene)tetrahydro-1H-pyrrolizin-7a(5H)-yl)methanol (98.8 mg, 3.0 equiv). The reaction was stirred at 100° C. for 12 hours. The mixture was diluted with water (10 mL) and extracted with ethyl acetate (2×10 mL). The combined organic layers were washed with brine (10 mL), dried over anhydrous sodium sulfate, concentrated and purified with prep-TLC (SiO₂, DCM/MeOH=10/1) to afford the title compound (50.0 mg, 38% yield) as yellow solid; LCMS (ESI, M+1, M+3): m/z=669.9, 671.9.

Step D. tert-butyl (Z)-(4-(6-chloro-4-(3-chloro-2-(dimethylcarbamoyl)-7,8-dihydro-4H-pyrazolo[1,5-a][1,4]diazepin-5(6H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of (Z)-5-(7-bromo-6-chloro-8-fluoro-2-((2-(fluoromethylene)tetrahydro-1H-pyrrolizin-7a(5H)-yl)methoxy)quinazolin-4-yl)-3-chloro-N,N-dimethyl-5,6,7,8-tetrahydro-4H-pyrazolo[1,5-a][1,4]diazepine-2-carboxamide (40.0 mg, 1.0 equiv) and tert-butyl N-[3-cyano-4-(5,5-dimethyl-1,3,2-dioxaborinan-2-yl)-7-fluoro-benzothiophen-2-yl]carbamate (36.1 mg, 1.5 equiv) in DMF (1.5 mL) were added Pd(DPEphos)Cl₂ (4.10 mg, 0.1 equiv) and Cs₂CO₃ (58.2 mg, 3.0 equiv). The reaction was degassed and purged with nitrogen for three times. The reaction was stirred at 110° C. for 1 hour under nitrogen atmosphere. The mixture was diluted with water (10 mL) and extracted with ethyl acetate (2×10 mL). The combined organic layers were washed with brine (10 mL), dried over anhydrous sodium sulfate, concentrated and purified with prep-TLC (SiO₂, DCM/MeOH=10/1) to afford the title compound (50.0 mg, 66% yield) as yellow solid; LCMS (ESI, M+1): m/z=882.3.

Step E. (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)guinazolin-4-yl)-3-chloro-N,N-dimethyl-5,6,7,8-tetrahydro-4H-pyrazolo[1,5-a][1,4]diazepine-2-carboxamide: To a solution of tert-butyl (Z)-(4-(6-chloro-4-(3-chloro-2-(dimethylcarbamoyl)-7,8-dihydro-4H-pyrazolo[1,5-a][1,4]diazepin-5(6H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate (50.0 mg, 1.0 equiv) in DCM (1.5 mL) was added TFA (1.5 mL). The reaction was stirred at 0° C. for 1 hour. The mixture was concentrated to remove solvent. The residue was diluted with ethyl acetate (10 mL), washed with saturated sodium bicarbonate solution (10 mL), dried over anhydrous sodium sulfate, concentrated and purified with prep-HPLC [column: Waters Xbridge 150×25 mm×5 μm; mobile phase: [water(NH₄HCO₃)−ACN]; gradient: 48%-78% B over 9 minutes] to afford the title compound (4.42 mg, 9.5% yield) as white solid; ¹H NMR (400 MHz, METHANOL-d₄) δ=7.96 (d, J=1.2 Hz, 1H), 7.23 (dd, J=5.2, 8.4 Hz, 1H), 7.09-7.03 (m, 1H), 6.90-6.65 (m, 1H), 5.16-5.01 (m, 2H), 4.48-4.32 (m, 6H), 4.18 (br d, J=14.8 Hz, 1H), 3.83 (br d, J=14.8 Hz, 1H), 3.55-3.47 (m, 1H), 3.16 (s, 3H), 3.13 (s, 3H), 3.05-2.97 (m, 1H), 2.88 (br d, J=15.2 Hz, 1H), 2.67-2.58 (m, 1H), 2.52-2.43 (m, 2H), 2.31-2.24 (m, 1H), 2.16-1.98 (m, 3H); LCMS (ESI, M+1): m/z=782.3.

### Example 162

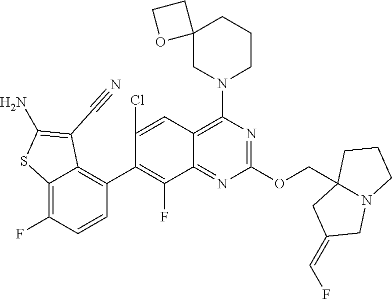 [SMILES: N#Cc1c(N)sc2c(F)ccc(-c3c(Cl)cc4c(N5CCCC6(CCO6)C5)nc(OCC56CCCN5C/C(=C\F)C6)nc4c3F)c12]

### (Z)-2-amino-4-(6-chloro-8-fluoro-2-((2-(fluoromethylene)tetrahydro-1H-pyrrolizin-7a(5H)-yl)methoxy)-4-(1-oxa-6-azaspiro[3.5]nonan-6-yl)quinazolin-7-yl)-7-fluorobenzo[b]thiophene-3-carbonitrile

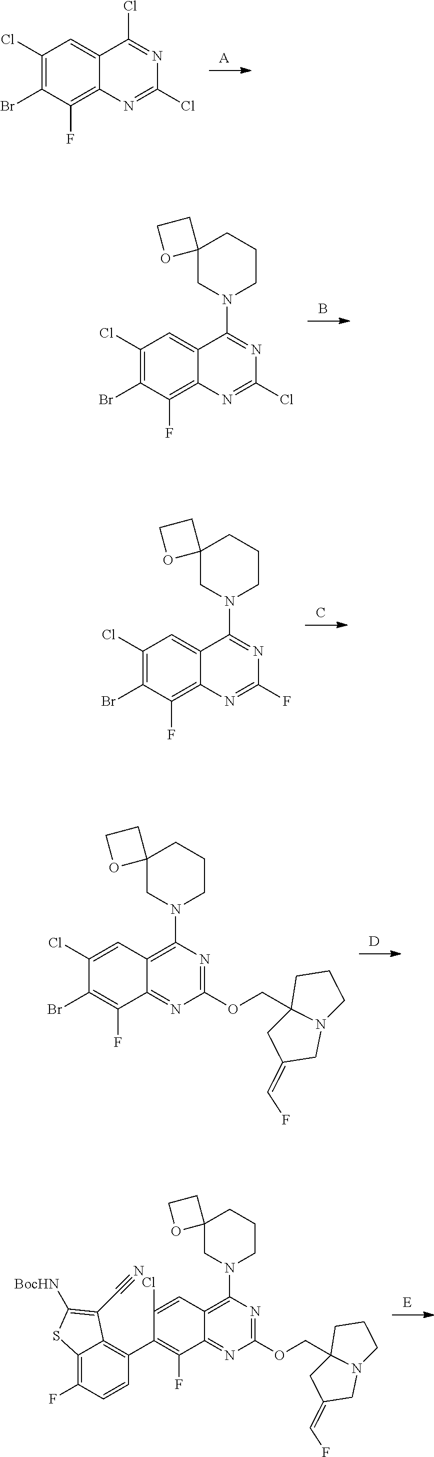

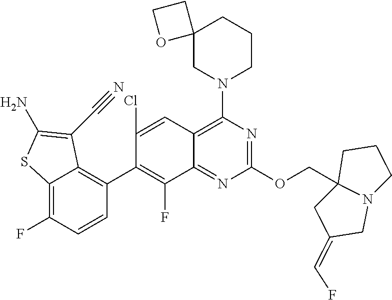 [SMILES: N#Cc1c(N)sc2c(F)ccc(-c3c(Cl)cc4c(N5CCCC6(CCO6)C5)nc(OCC56CCCN5C/C(=C\F)C6)nc4c3F)c12]

Step A. 6-(7-bromo-2,6-dichloro-8-fluoroqiuinazolin-4-yl)-1-oxa-6-azaspiro[3.5]nonane: To a solution of 7-bromo-2,4,6-trichloro-8-fluoroquinazoline (457 mg, 1.0 equiv) and DIEA (715 mg, 4.0 equiv) in DCM (5 mL) was added 1-oxa-6-azaspiro[3.5]nonane (300 mg, 1.0 equiv, oxalic acid) at −40° C. The reaction was stirred at −40° C. for 1 hour. The mixture was diluted with water (20 mL) and extracted with DCM (2×20 mL). The combined organic layers were washed with brine (20 mL), dried over anhydrous sodium sulfate, concentrated, and purified with column chromatography (SiO₂, petroleum ether/ethyl acetate=10/1 to 5/1) to afford the title compound (277 mg, 47% yield) as yellow solid; LCMS (ESI, M+1, M+3): m/z=420.0, 422.0.

Step B. 6-(7-bromo-6-chloro-2,8-difluoroquinazolin-4-yl)-1-oxa-6-azaspiro[3.5]nonane: To a solution of 6-(7-bromo-2,6-dichloro-8-fluoroquinazolin-4-yl)-1-oxa-6-azaspiro[3.5]nonane (277 mg, 1.0 equiv) in DMSO (10 mL) were added KF (764 mg, 20 equiv) and 18-crown-6 ether (17.4 mg, 0.1 equiv). The reaction was stirred at 140° C. for 0.5 hours. The mixture was diluted with water (50 mL) and extracted with EtOAc (2×45 mL). The combined organic layers were washed with brine (50 mL), dried over anhydrous sodium sulfate, concentrated, and purified with column chromatography (SiO₂, petroleum ether/ethyl acetate=5/1 to 3/1) to afford the title compound (226 mg, 76% yield) as yellow solid; LCMS (ESI, M+1, M+3): m/z=404.1, 406.1.

Step C. (Z)-6-(7-bromo-6-chloro-8-fluoro-2-((2-(fluoromethylene)tetrahydro-1H-pyrrolizin-7a(5H)-yl)methoxy)guinazolin-4-yl)-1-oxa-6-azaspiro[3.5]nonane: To a mixture of 6-(7-bromo-6-chloro-2,8-difluoroquinazolin-4-yl)-1-oxa-6-azaspiro[3.5]nonane (100 mg, 1.0 equiv) in DMSO (0.5 mL) was added (Z)-(2-(fluoromethylene)tetrahydro-1H-pyrrolizin-7a(5H)-yl)methanol (113 mg, 3.0 equiv). The reaction was stirred at 100° C. for 12 hours. The mixture was diluted with water (10 mL) and extracted with DCM (2×10 mL). The combined organic layers were washed with brine (10 mL), dried over anhydrous sodium sulfate, concentrated, and purified with prep-TLC (SiO₂, DCM/MeOH=10/1) to afford the title compound (56.0 mg, 43% yield) as white gum; LCMS (ESI, M+1, M+3): m/z=555.1, 557.1.

Step D. tert-butyl (Z)-(4-(6-chloro-8-fluoro-2-((2-(fluoromethylene)tetrahydro-1H-pyrrolizin-7a(5H)-yl)methoxy)-4-(1-oxa-6-azaspiro[3.5]nonan-6-yl)quinazolin-7-yl)-3-cyano-7-fluorobenzo[b]thiophen-2-yl)carbamate: To a solution of (Z)-6-(7-bromo-6-chloro-8-fluoro-2-((2-(fluoromethylene)tetrahydro-1H-pyrrolizin-7a(5H)-yl)methoxy)quinazolin-4-yl)-1-oxa-6-azaspiro[3.5]nonane (46.0 mg, 1.0 equiv) and tert-butyl (3-cyano-4-(5,5-dimethyl-1,3,2-dioxaborinan-2-yl)-7-fluorobenzo[b]thiophen-2-yl)carbamate (50.2 mg, 1.5 equiv) in DMF (1 mL) were added Pd(DPEphos)Cl₂ (5.69 mg, 0.1 equiv) and Cs₂CO₃ (80.9 mg, 3.0 equiv). The reaction was degassed and purged with nitrogen for 3 times. The reaction was stirred at 110° C. for 5 hours under nitrogen atmosphere. The mixture was diluted with water (10 mL) and extracted with DCM (3×10 mL). The combined organic layers were washed with brine (10 mL), dried over anhydrous sodium sulfate, concentrated, and purified with prep-TLC (SiO₂, DCM/MeOH=10/1) and reversed phase flash chromatography (0.1% FA condition) to afford the title compound (18.0 mg, 26% yield) as yellow solid; LCMS (ESI, M+1): m/z=767.4.

Step E. (Z)-2-amino-4-(6-chloro-8-fluoro-2-((2-(fluoromethylene)tetrahydro-1H-pyrrolizin-7a(5H)-yl)methoxy)-4-(1-oxa-6-azaspiro[3.5]nonan-6-yl)quinazolin-7-yl)-7-fluorobenzo[b]thiophene-3-carbonitrile: To a solution of tert-butyl (Z)-(4-(6-chloro-8-fluoro-2-((2-(fluoromethylene)tetrahydro-1H-pyrrolizin-7a(5H)-yl)methoxy)-4-(1-oxa-6-azaspiro[3.5]nonan-6-yl)quinazolin-7-yl)-3-cyano-7-fluorobenzo[b]thiophen-2-yl)carbamate (20.0 mg, 1.0 equiv) in DCM (0.6 mL) was added TFA (0.2 mL). The reaction was stirred at 0° C. for 4 hours. The pH of the mixture was adjusted to 7 with TEA at −40° C. and concentrated. The residue was diluted with water (5 mL) and extracted with EtOAc (3×5 mL). The combined organic layers were washed with brine (5 mL), dried over anhydrous sodium sulfate, concentrated and purified with prep-HPLC [Waters Xbridge 150×25 mm×5 um; A: water(NH₄HCO₃), B: ACN, B %: 54%-84% over 9 min] and lyophilized to afford the title compound (3.92 mg, 22% yield) as yellow solid; ¹H NMR (400 MHz, METHANOL-d₄) δ=8.26-8.13 (m, 1H), 7.26-7.18 (m, 1H), 7.07-7.01 (m, 1H), 6.76-6.52 (m, 1H), 4.69-4.51 (m, 3H), 4.48-4.35 (m, 1H), 4.34-4.23 (m, 2H), 4.17-4.16 (m, 1H), 4.21-4.06 (m, 1H), 3.85 (br d, J=15.6 Hz, 1H), 3.72-3.59 (m, 1H), 3.50-3.36 (m, 2H), 3.19-3.11 (m, 1H), 2.80-2.66 (m, 2H), 2.54-2.41 (m, 3H), 2.26-2.10 (m, 2H), 2.01-1.81 (m, 5H); LCMS (ESI, M+1): m/z=667.3.

### Example 163

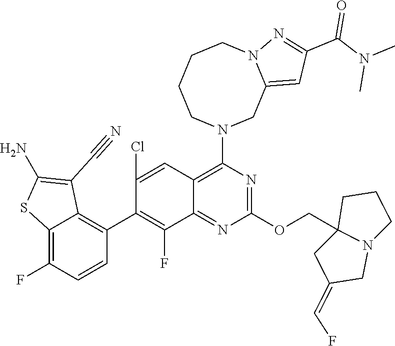 [SMILES: CN(C)C(=O)c1cc2n(n1)CCCCN(c1nc(OCC34CCCN3C/C(=C\F)C4)nc3c(F)c(-c4ccc(F)c5sc(N)c(C#N)c45)c(Cl)cc13)C2]

### (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide

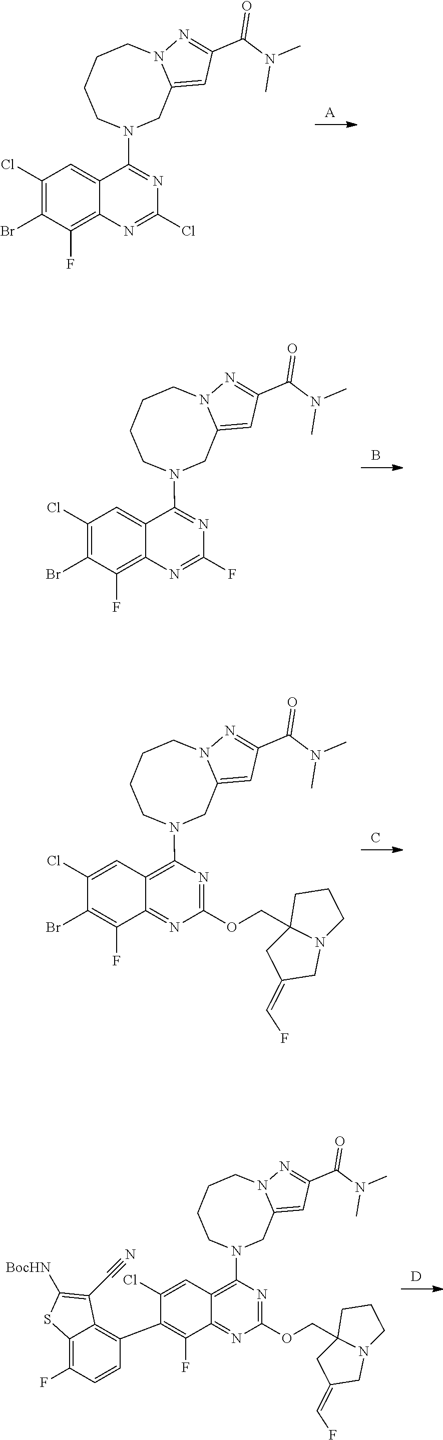

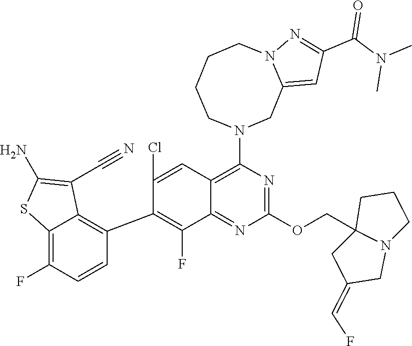 [SMILES: CN(C)C(=O)c1cc2n(n1)CCCCN(c1nc(OCC34CCCN3C/C(=C\F)C4)nc3c(F)c(-c4ccc(F)c5sc(N)c(C#N)c45)c(Cl)cc13)C2]

Step A. 5-(7-bromo-6-chloro-2,8-difluoroquinazolin-5-yl)-N,N-dimethyl-4,5,6,7,8,9-hexahydropyrazolo[1,5-a][1,4]diazocine-2-carboxamide: To a solution of 5-(7-bromo-2,6-dichloro-8-fluoro-quinazolin-5-yl)-N,N-dimethyl-6,7,8,9-tetrahydro-4H-pyrazolo[1,5-a][1,4]diazocine-2-carboxamide (500 mg, 1.0 equiv) in DMSO (5 mL) was added KF (1.13 g, 20 equiv). The reaction was stirred at 140° C. for 2 hours. The mixture was diluted with water (25 mL) and extracted with ethyl acetate (3×25 mL). The combined organic layers were washed with brine (30 mL), dried over anhydrous sodium sulfate, concentrated, and triturated with DCM (5 mL) at 25° C. to afford the title compound (250 mg, 46% yield) as white solid; LCMS (ESI, M+1, M+3): m/z=499.2, 501.2.

Step B. (Z)-5-(7-bromo-6-chloro-8-fluoro-2-((2-(fluoromethylene)tetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide: To a solution of 5-(7-bromo-6-chloro-2,8-difluoro-quinazolin-5-yl)-N,N-dimethyl-6,7,8,9-tetrahydro-4H-pyrazolo[1,5-a][1,4]diazocine-2-carboxamide (150 mg, 1.0 equiv) in DMSO (1 mL) was added (Z)-(2-(fluoromethylene)tetrahydro-1H-pyrrolizin-7a(5H)-yl)methanol (154 mg, 3.0 equiv). The reaction was stirred at 100° C. for 8 hours. The mixture was diluted with water (10 mL) and extracted with ethyl acetate (3×10 mL). The combined organic layers were washed with brine (10 mL), dried over anhydrous sodium sulfate, concentrated, and purified with prep-TLC (SiO₂, petroleum ether/ethyl acetate=0/1) to afford the title compound (80.0 mg, 37% yield) as yellow oil; LCMS (ESI, M+1, M+3): m/z=650.3, 652.2.

Step C. tert-butyl (Z)-(4-(6-chloro-4-(2-(dimethylcarbamoyl)-6,7,8,9-tetrahydropyrazolo[1,5-a][1,4]diazocin-5(4H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of (Z)-5-(7-bromo-6-chloro-8-fluoro-2-((2-(fluoromethylene)tetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide (80.0 mg, 1.0 equiv) and tert-butyl N-[3-cyano-4-(5,5-dimethyl-1,3,2-dioxaborinan-2-yl)-7-fluoro-benzothiophen-2-yl]carbamate (59.6 mg, 1.2 equiv) in toluene (2 mL) were added Pd(DPEphos)Cl₂ (8.45 mg, 0.1 equiv) and Cs₂CO₃ (80.1 mg, 2.0 equiv). The reaction was degassed and purged with nitrogen for 3 times. The reaction was stirred at 110° C. for 2 hours under nitrogen atmosphere. The mixture was diluted with water (10 mL) and extracted with ethyl acetate (3×10 mL). The combined organic layers were washed with brine (10 mL), dried over anhydrous sodium sulfate, concentrated, and purified with prep-TLC (SiO₂, DCM/MeOH=10/1) to afford the title compound (55.0 mg, 39% yield) as yellow oil; LCMS (ESI, M+1): m/z=862.4.

Step D. (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-4,5,6,7,8,9-hexahydropyrazolo[1,5-a][1,4]diazocine-2-carboxamide: To a solution of tert-butyl (Z)-(4-(6-chloro-4-(2-(dimethylcarbamoyl)-6,7,8,9-tetrahydropyrazolo[1,5-a][1,4]diazocin-5(4H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate (55.0 mg, 1.0 equiv) in DCM (2 mL) was added TFA (2 mL). The reaction was stirred at 0° C. for 2 hours. The mixture was concentrated to remove solvent. The residue was diluted with ethyl acetate (10 mL), washed with saturated sodium bicarbonate solution (10 mL) and brine (10 mL), dried over anhydrous sodium sulfate, concentrated and purified with prep-HPLC [column: Waters Xbridge 150×25 mm×5 μm; mobile phase: [water (ammonia hydroxide v/v)−ACN]; gradient: 45%-75% B over min] to afford the title compound (6.93 mg, 14% yield) as white solid; ¹H NMR (400 MHz, METHANOL-d₄) δ=7.86-7.79 (m, 1H), 7.19 (dd, J=5.2, 8.4 Hz, 1H), 7.06-6.99 (m, 1H), 6.75-6.49 (m, 2H), 5.22 (s, 2H), 4.48 (br t, J=5.6 Hz, 2H), 4.28-4.16 (m, 2H), 4.03 (br s, 2H), 3.81 (br d, J=15.2 Hz, 1H), 3.44 (br d, J=15.6 Hz, 1H), 3.33 (s, 3H), 3.15-3.08 (m, 4H), 2.73-2.64 (m, 2H), 2.41 (br d, J=15.6 Hz, 1H), 2.09 (s, 1H), 1.97 (br d, J=5.6 Hz, 4H), 1.90-1.77 (m, 2H); LCMS (ESI, M+1): m/z=762.4.

### Example 164

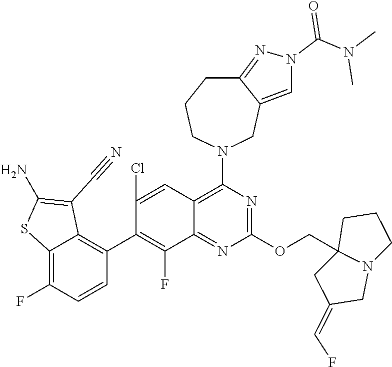 [SMILES: CN(C)C(=O)n1cc2c(n1)CCCN(c1nc(OCC34CCCN3C/C(=C\F)C4)nc3c(F)c(-c4ccc(F)c5sc(N)c(C#N)c45)c(Cl)cc13)C2]

### (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide

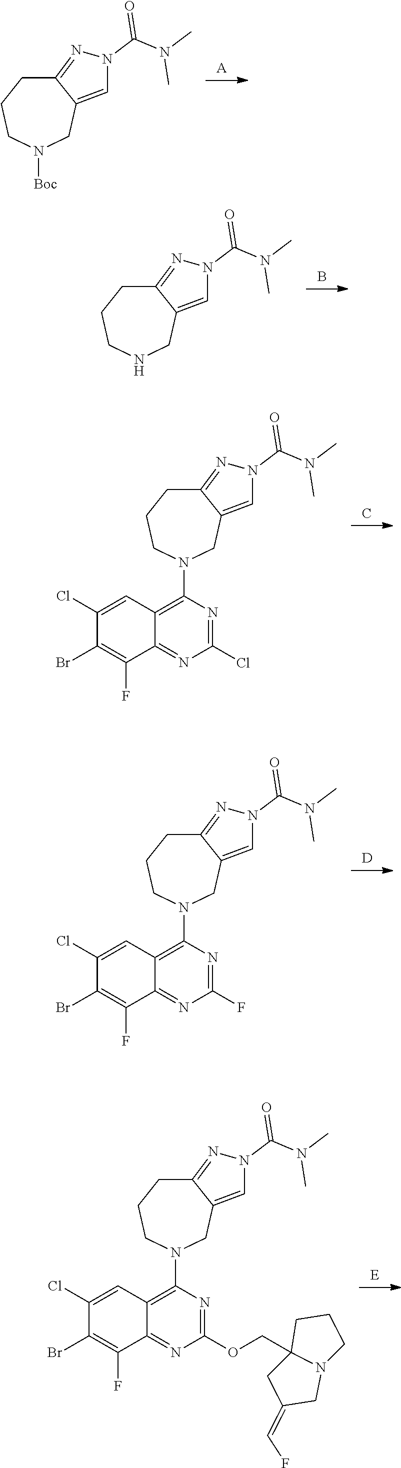

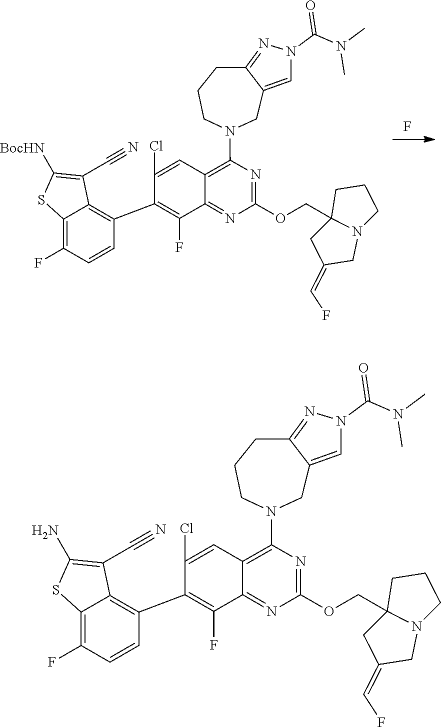

Step A. N,N-dimethyl-5,6,7,8-tetrahydropyrazolo[4,3-c]azepine-2(4H)-carboxamide: To a solution of tert-butyl 2-(dimethylcarbamoyl)-4,6,7,8-tetrahydropyrazolo[4,3-c]azepine-5-carboxylate (1.10 g, 1.0 equiv) in ACN (20 mL) was added HCl.dioxane (5 mL). The reaction was stirred at 0° C. for 1 hour. The mixture was concentrated to remove solvent. The residue was dissolved in MeOH (20 mL), and its pH was adjusted to 7-8 with sodium bicarbonate. The mixture was filtered and concentrated. The residue was dissolved in DCM/MeOH=10/1 (20 mL), filtered and concentrated to afford the title compound (940 mg, crude) as white solid; LCMS (ESI, M+1): m/z=209.1.

Step B. 5-(7-bromo-2,6-dichloro-8-fluoroquinazolin-4-yl)-N,N-dimethyl-5,6,7,8-tetrahydropyrazolo[4,3-c]azepine-2(4H)-carboxamide: To a solution of N,N-dimethyl-5,6,7,8-tetrahydropyrazolo[4,3-c]azepine-2(4H)-carboxamide (850 mg, 1.0 equiv) and 7-bromo-2,4,6-trichloro-8-fluoro-quinazoline (1.35 g, 1.0 equiv) in DCM (15 mL) was added DIEA (1.58 g, 3.0 equiv). The reaction was stirred at −40° C. for 1 hour. The mixture was diluted with water (10 mL) and extracted with DCM (2×10 mL). The combined organic layers were washed with brine (10 mL), dried over anhydrous sodium sulfate, concentrated, and purified with column chromatography (SiO₂, petroleum ether/ethyl acetate=5/1 to 0/1) to afford the title compound (750 mg, 37% yield) as yellow solid; LCMS (ESI, M+1, M+3): m/z=501.1, 503.1.

Step C. 5-(7-bromo-6-chloro-2,8-difluoroquinazolin-4-yl)-N,N-dimethyl-5,6,7,8-tetrahydropyrazolo[4,3-c]azepine-2(4H)-carboxamide: To a solution of 5-(7-bromo-2,6-dichloro-8-fluoroquinazolin-4-yl)-N,N-dimethyl-5,6,7,8-tetrahydropyrazolo[4,3-c]azepine-2(4H)-carboxamide (300 mg, 1.0 equiv) in DMSO (4 mL) was added KF (694 mg, 20 equiv). The reaction was stirred at 140° C. for 1 hour. The mixture was diluted with water (10 mL) and extracted with ethyl acetate (2×10 mL). The combined organic layers were washed with brine (10 mL), dried over anhydrous sodium sulfate, concentrated, and purified with prep-TLC (SiO₂, petroleum ether/ethyl acetate=1/1) to afford the title compound (110 mg, 38% yield) as yellow solid; LCMS (ESI, M+1, M+3): m/z=485.0, 487.0.

Step D. (Z)-5-(7-bromo-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: To a mixture of 5-(7-bromo-6-chloro-2,8-difluoro-quinazolin-4-yl)-N,N-dimethyl-4,6,7,8-tetrahydropyrazolo[4,3-c]azepine-2-carboxamide (100 mg, 1.0 equiv) in DMSO (0.1 mL) was added (Z)-(2-(fluoromethylene)tetrahydro-1H-pyrrolizin-7a(5H)-yl)methanol (106 mg, 3.0 equiv). The reaction was stirred at 100° C. for 12 hours. The mixture was diluted with water (10 mL) and extracted with ethyl acetate (2×10 mL). The combined organic layers were washed with brine (10 mL), dried over anhydrous sodium sulfate, concentrated and purified with prep-TLC (SiO₂, DCM/MeOH=10/1) to afford the title compound (70.0 mg, 53% yield) as yellow solid; LCMS (ESI, M+1, M+3): m/z=636.1, 638.1.

Step E. tert-butyl (Z)-(4-(6-chloro-4-(2-(dimethylcarbamoyl)-2,6,7,8-tetrahydropyrazolo[4,3-c]azepin-5(4H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of (Z)-5-(7-bromo-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide (70.0 mg, 1.0 equiv) and tert-butyl (3-cyano-4-(5,5-dimethyl-1,3,2-dioxaborinan-2-yl)-7-fluorobenzo[b]thiophen-2-yl)carbamate (53.3 mg, 1.2 equiv) in toluene (1 mL) were added Pd(DPEphos)Cl₂ (7.56 mg, 0.1 equiv) and Cs₂CO₃ (71.6 mg, 2.0 equiv). The reaction was degassed and purged with nitrogen for three times. The reaction was stirred at 110° C. for 1 hour under nitrogen atmosphere. The mixture was diluted with water (20 mL) and extracted with ethyl acetate (2×20 mL). The combined organic layers were washed with brine (20 mL), dried over anhydrous sodium sulfate, concentrated, and purified with prep-TLC (SiO₂, DCM/MeOH=10/1) to afford the title compound (50.0 mg, 54% yield) as yellow solid; LCMS (ESI, M+1): m/z=848.3.

Step F. (Z)-5-(7-(2-amino-3-cyano-7-fluorobenzo[b]thiophen-4-yl)-6-chloro-8-fluoro-2-((2-(fluoromethylene)tetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: To a solution of tert-butyl (Z)-(4-(6-chloro-4-(2-(dimethylcarbamoyl)-2,6,7,8-tetrahydropyrazolo[4,3-c]azepin-5(4H)-yl)-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate (50.0 mg, 1.0 equiv) in DCM (1.5 mL) was added TFA (1.5 mL). The reaction was stirred at 0° C. for 1 hour. The mixture was concentrated to remove solvent. The residue was dissolved in ethyl acetate (10 mL), washed with saturated sodium bicarbonate solution (10 mL), dried over anhydrous sodium sulfate, concentrated and purified with prep-HPLC [column: Waters Xbridge 150×25 mm×5 m; mobile phase: [water(NH₄HCO₃)−ACN]; gradient: 55%-85% B over 9 minutes] to afford the title compound (7.09 mg, 16% yield) as white solid; ¹H NMR (400 MHz, METHANOL-d₄) δ=8.16 (s, 1H), 7.99 (d, J=1.6 Hz, 1H), 7.20 (dd, J=4.8, 8.0 Hz, 1H), 7.03 (dd, J=8.4, 9.2 Hz, 1H), 6.77-6.51 (m, 1H), 4.96 (s, 2H), 4.31-4.19 (m, 4H), 3.88 (br d, J=14.4 Hz, 1H), 3.49 (br d, J=14.4 Hz, 1H), 3.19 (s, 6H), 3.02-2.96 (m, 2H), 2.77-2.68 (m, 2H), 2.44 (br d, J=15.6 Hz, 1H), 2.32-1.76 (m, 7H); LCMS (ESI, M+1): m/z=748.2.

### Example 165

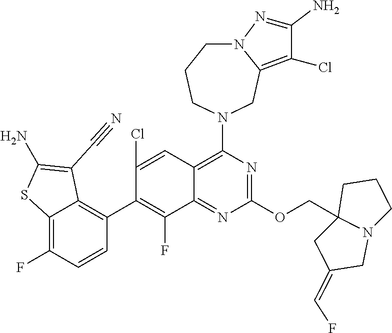 [SMILES: N#Cc1c(N)sc2c(F)ccc(-c3c(Cl)cc4c(N5CCCn6nc(N)c(Cl)c6C5)nc(OCC56CCCN5C/C(=C\F)C6)nc4c3F)c12]

### (Z)-2-amino-4-(4-(2-amino-3-chloro-7,8-dihydro-4H-pyrazolo[1,5-a][1,4]diazepin-5(6H)-yl)-6-chloro-8-fluoro-2-((2-(fluoromethylene)tetrahydro-1H-pyrrolizin-7a(5H)-yl)methoxy)quinazolin-7-yl)-7-fluorobenzo[b]thiophene-3-carbonitrile

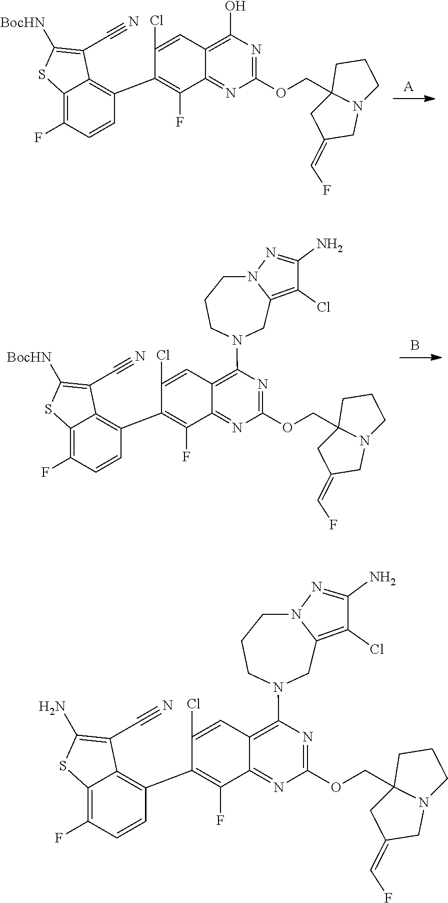

Step A. tert-butyl (Z)-(4-(4-(2-amino-3-chloro-7,8-dihydro-4H-pyrazolo[1,5-a][1,4]diazepin-5(6H)-yl)-6-chloro-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate: To a solution of tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (90.0 mg, 1.0 equiv) in DMSO (0.9 mL) were added PyBOP (107 mg, 1.5 equiv) and TEA (48.4 mg, 3.5 equiv). The reaction was stirred at 30° C. for 0.3 hours and 3-chloro-5,6,7,8-tetrahydro-4H-pyrazolo[1,5-a][1,4]diazepin-2-amine (51.0 mg, 2.0 equiv) was added. The reaction was stirred at 30° C. for 2 hours. The mixture was partitioned between water (10 mL) and ethyl acetate (3×15 mL). The organic phase was separated, washed with brine (2×10 mL), dried over sodium sulfate, concentrated, and purified with prep-HPLC (0.1% NH₃H₂O condition) to afford the title compound (70.0 mg, 52% yield) as yellow oil; LCMS (ESI, M+1): m/z=826.5.

Step B. (Z)-2-amino-4-(4-(2-amino-3-chloro-7,8-dihydro-4H-pyrazolo[1,5-a][1,4]diazepin-5(6H)-yl)-6-chloro-8-fluoro-2-((2-(fluoromethylene)tetrahydro-1H-pyrrolizin-7a(5H)-yl)methoxy)quinazolin-7-yl)-7-fluorobenzo[b]thiophene-3-carbonitrile: To a solution of tert-butyl (Z)-(4-(4-(2-amino-3-chloro-7,8-dihydro-4H-pyrazolo[1,5-a][1,4]diazepin-5(6H)-yl)-6-chloro-8-fluoro-2-((2-(fluoromethylene)tetrahydro-1H-pyrrolizin-7a(5H)-yl)methoxy)quinazolin-7-yl)-3-cyano-7-fluorobenzo[b]thiophen-2-yl)carbamate (57.0 mg, 1.0 equiv) in DCM (0.6 mL) was added TFA (0.6 mL). The reaction was stirred at 25° C. for 0.5 hours. The mixture was concentrated, diluted with saturated sodium bicarbonate solution (10 mL), and extracted with ethyl acetate (3×10 mL). The combined organic layers were washed with brine (20 mL), dried over anhydrous sodium sulfate, concentrated and purified with prep-HPLC (column: Waters Xbridge 150×25 mm×5 um; mobile phase: [water (NH₄HCO₃)−ACN]; gradient:48%-78% B over 9 min) to afford the title compound (7.10 mg, 16% yield) as white solid; ¹H NMR (400 MHz, METHANOL-d₄) δ=7.87 (s, 1H), 7.20 (dd, J=5.2, 8.4 Hz, 1H), 7.04 (t, J=8.8 Hz, 1H), 6.76-6.51 (m, 1H), 4.67-4.62 (m, 1H), 4.32-4.17 (m, 6H), 3.90-3.81 (m, 1H), 3.54-3.40 (m, 2H), 3.20-3.12 (m, 1H), 2.75-2.66 (m, 2H), 2.49-2.34 (m, 3H), 2.18-2.07 (m, 1H), 2.00-1.89 (m, 2H), 1.88-1.79 (m, 1H); LCMS (ESI, M+1): m/z=726.3.

### Example 166

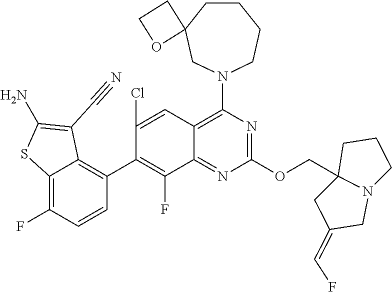 [SMILES: N#Cc1c(N)sc2c(F)ccc(-c3c(Cl)cc4c(N5CCCCC6(CCO6)C5)nc(OCC56CCCN5C/C(=C\F)C6)nc4c3F)c12]

### (Z)-2-amino-4-(6-chloro-8-fluoro-2-((2-(fluoromethylene)tetrahydro-1H-pyrrolizin-7a(5H)-yl)methoxy)-4-(1-oxa-6-azaspiro[3.6]decan-6-yl)quinazolin-7-yl)-7-fluorobenzo[b]thiophene-3-carbonitrile

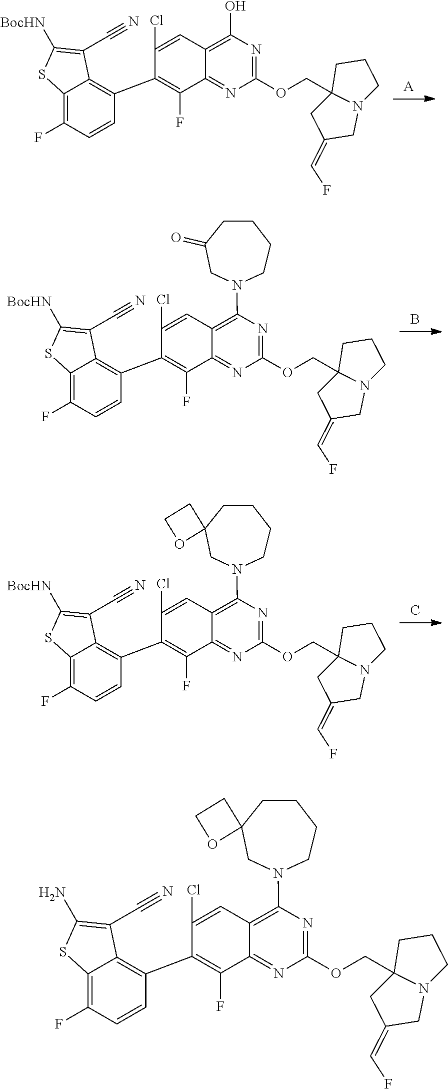

Step A. tert-butyl (Z)-(4-(6-chloro-8-fluoro-2-((2-(fluoromethylene)tetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate: To a solution of tert-butyl (Z)-(4-(6-chloro-8-fluoro-2-((2-(fluoromethylene)tetrahydro-1H-pyrrolizin-7a(5H)-yl)methoxy)-4-hydroxyquinazolin-7-yl)-3-cyano-7-fluorobenzo[b]thiophen-2-yl)carbamate (166 mg, 1.0 equiv) in DMSO (4 mL) were added TEA (204 mg, 8.0 equiv) and PyBOP (197 mg, 1.5 equiv). The reaction was stirred at 30° C. for 0.5 hours and azepan-3-one (188 mg, 5.0 equiv, HCl) was added. The reaction was stirred at 30° C. for 12 hours. The mixture was quenched with water (30 mL) at 20° C. and extracted with EtOAc (2×60 mL). The combined organic layers were washed with brine (50 mL), dried over anhydrous sodium sulfate, concentrated, and purified with prep-TLC (SiO₂, DCM/MeOH=10/1) to afford the title compound (101 mg, 47% yield) as yellow solid; LCMS (ESI, M+1): m/z=753.4.

Step B. tert-butyl (Z)-(4-(6-chloro-8-fluoro-2-((2-(fluoromethylene)tetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate: To a solution of trimethylsulfoxonium iodide (79.8 mg, 3.0 equiv) in t-BuOH (1.8 mL) was added t-BuOK (40.7 mg, 3.0 equiv) at 20° C. The reaction was stirred at 50° C. for 1.5 hours. A solution of tert-butyl (Z)-(4-(6-chloro-8-fluoro-2-((2-(fluoromethylene)tetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate (91.0 mg, 1.0 equiv) in t-BuOH (1.8 mL) was added. The reaction was stirred at 50° C. for 12 hours. The mixture was quenched with water (10 mL) at 20° C. and extracted with EtOAc (2×20 mL). The combined organic layers were washed with brine (10 mL), dried over anhydrous sodium sulfate, concentrated, and purified with prep-TLC (SiO₂, DCM/MeOH=10/1) to afford the title compound (11.0 mg, 11% yield) as yellow solid; LCMS (ESI, M+1): m/z=781.3.

Step C. (Z)-2-amino-4-(6-chloro-8-fluoro-2-((2-(fluoromethylene)tetrahydro-1H-pyrrolizin-7a(5H)-yl)methoxy)-4-(1-oxa-6-azaspiro[3.6]decan-6-yl)quinazolin-7-yl)-7-fluorobenzo[b]thiophene-3-carbonitrile: To a solution of tert-butyl (Z)-(4-(6-chloro-8-fluoro-2-((2-(fluoromethylene)tetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate (10.0 mg, 1.0 equiv) in DCM (1.8 mL) was added TFA (184 mg, 126 equiv) at 0° C. The reaction was stirred at 0° C. for 6 hours. The mixture was quenched with TEA (1.5 mL) at −40° C., diluted with water (10 mL) and extracted with EtOAc (2×20 mL). The combined organic layers were washed with brine (10 mL), dried over anhydrous sodium sulfate, concentrated and purified with prep-HPLC (column: Waters Xbridge 150*25 mm*5 um; mobile phase: [water(NH₄HCO₃)−ACN];gradient:62%-92% B over 9 min) to afford the title compound (3.15 mg, 36% yield) as white solid; ¹H NMR (400 MHz, METHANOL-d₄) δ=8.59 (d, J=1.6 Hz, 1H), 7.21 (dd, J=5.2, 8.4 Hz, 1H), 7.09-7.00 (m, 1H), 6.79-6.52 (m, 2H), 4.36-4.24 (m, 4H), 3.90-3.78 (m, 3H), 3.52-3.39 (m, 2H), 3.21-3.14 (m, 1H), 2.81-2.67 (m, 2H), 2.49-2.39 (m, 5H), 2.20-2.13 (m, 1H), 2.09-1.80 (m, 8H); LCMS (ESI, M+1): m/z=681.2.

### Example 167

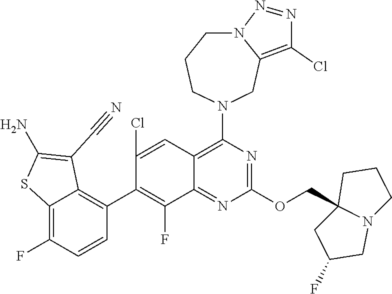 [SMILES: N#Cc1c(N)sc2c(F)ccc(-c3c(Cl)cc4c(N5CCCn6nnc(Cl)c6C5)nc(OC[C@@]56CCCN5C[C@H](F)C6)nc4c3F)c12]

### 2-amino-4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

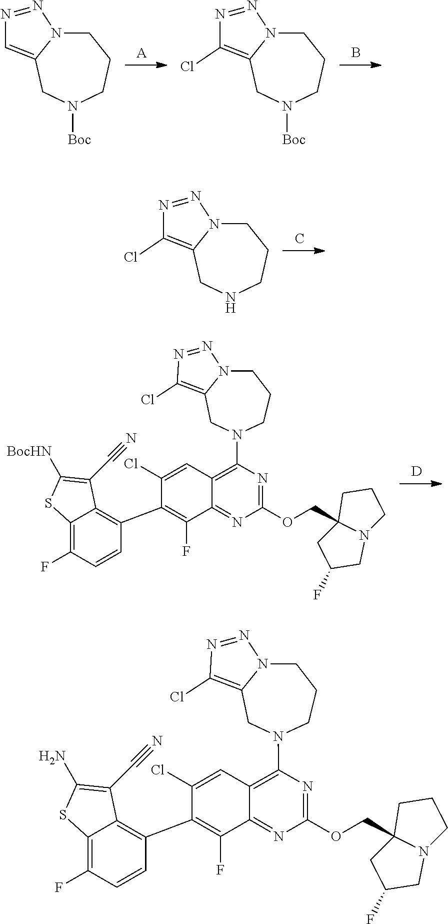

Step A. tert-butyl 3-chloro-7,8-dihydro-4H-[1,2,3]triazolo[1,5-a][1,4]diazepine-5(6H)-carboxylate: To a solution of tert-butyl 7,8-dihydro-4H-[1,2,3]triazolo[1,5-a][1,4]diazepine-5(6H)-carboxylate (270 mg, 1.0 equiv) in MeCN (10 mL) was added NCS (181 mg, 1.2 equiv). The reaction was stirred at 60° C. for 1 hour. The mixture was diluted water (10 mL) and extracted with EtOAc (3×10 mL). The combined organic layers were dried over anhydrous sodium sulfate, concentrated and purified with reversed phase flash chromatography [C18, 0.1% formic acid condition] to afford the title compound (250 mg, 81% yield) as yellow solid; LCMS (ESI, M+1): m/z=272.9.

Step B. 3-chloro-5,6,7,8-tetrahydro-4H-[1,2,3]triazolo[1,5-a][1,4]diazepine: To a solution of tert-butyl 3-chloro-7,8-dihydro-4H-[1,2,3]triazolo[1,5-a][1,4]diazepine-5(6H)-carboxylate (250 mg, 1.0 equiv) in MeOH (1 mL) was added HCl.dioxane (2.5 mL). The reaction was stirred at 0° C. for 1 hour. The mixture was concentrated and stirred with NaHCO₃(300 mg) in MeOH (2 mL) at 25° C. for 1 hour. The mixture was filtered and concentrated to afford the title compound (180 mg, crude) as yellow solid.

Step C. tert-butyl (4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) in DMSO (1 mL) was added TEA (47.0 mg, 3.0 equiv) and PyBOP (121 mg, 1.5 equiv). The mixture was stirred at 30° C. for 1 hour and 3-chloro-5,6,7,8-tetrahydro-4H-[1,2,3]triazolo[1,5-a][1,4]diazepine (53.4 mg, 2.0 equiv) was added. The reaction was stirred at 30° C. for 12 hours. The mixture was diluted water (10 mL) and extracted with EtOAc (3×10 mL). The combined organic layers were dried over anhydrous sodium sulfate, concentrated, and purified with reversed phase flash chromatography [C18, 0.1% formic acid condition] to afford the title compound (80 mg, 64% yield) as yellow solid; LCMS (ESI, M+1): m/z=800.2.

Step D. 2-amino-4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (70 mg, 1.0 equiv) in DCM (0.25 mL) was added TFA (1.54 g, 154 equiv). The reaction was stirred at 20° C. for 0.5 hours. The pH of the mixture was adjusted 8 with saturated NaHCO₃ aqueous solution (10 mL) at 0° C. and extracted with DCM (3×5.0 mL). The combined organic layers were dried over anhydrous sodium sulfate, concentrated and purified with prep-HPLC [Waters Xbridge 150×25 mm×5 μm; A: water (ammonia hydroxide v/v, B: ACN; B %:45%-75% over 9 min] to afford the title compound (12.4 mg, 20% yield) as white solid; ¹H NMR (400 MHz, METHANOL-d₄) δ=7.90 (s, 1H), 7.24-7.15 (m, 1H), 7.07-7.00 (m, 1H), 5.40-5.21 (m, 1H), 5.12-4.97 (m, 2H), 4.69-4.61 (m, 2H), 4.45-4.29 (m, 2H), 4.23-4.09 (m, 2H), 3.33 (br d, J=3.6 Hz, 1H), 3.25-3.11 (m, 2H), 3.04-2.96 (m, 1H), 2.46-2.21 (m, 3H), 2.20-2.05 (m, 2H), 2.04-1.83 (m, 3H); LCMS (ESI, M+1): m/z=700.1.

### Example 168

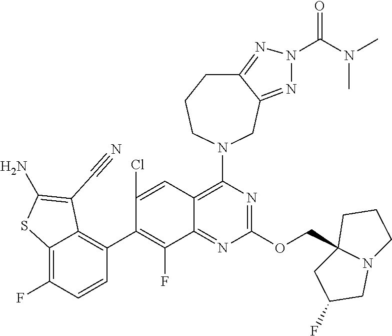 [SMILES: CN(C)C(=O)n1nc2c(n1)CN(c1nc(OC[C@@]34CCCN3C[C@H](F)C4)nc3c(F)c(-c4ccc(F)c5sc(N)c(C#N)c45)c(Cl)cc13)CCC2]

### 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-[1,2,3]triazolo[4,5-c]azepine-2(4H)-carboxamide

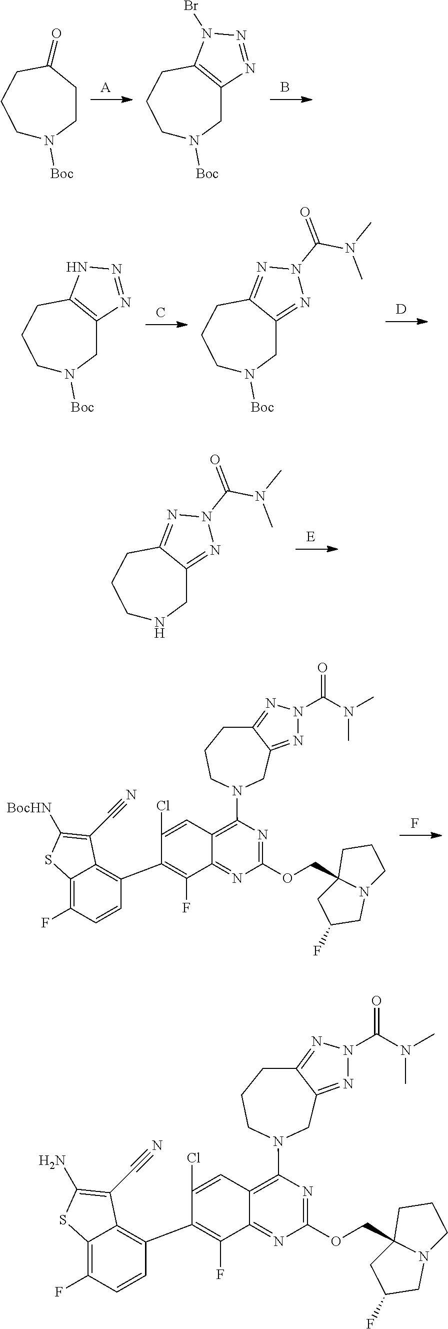

Step A. tert-butyl 1-benzyl-4,6,7,8-tetrahydro-[1,2,3]triazolo[4,5-c]azepine-5(1H)-carboxylate: To a solution of tert-butyl 4-oxoazepane-1-carboxylate (5.00 g, 1.0 equiv) in toluene (30 mL) at 100° C. was added dropwise azidobenzene (3.35 g, 1.2 equiv). Then pyrrolidine (1.67 g, 1.0 equiv) was added. The reaction was stirred for 1.2 hours at 100° C. The mixture was diluted with water (50 mL) and extracted with EtOAc (2×50 mL). The combined organic layers were washed with brine (30 mL), dried over anhydrous sodium sulfate, concentrated and purified with reversed phase flash chromatography [C18, 0.1% formic acid condition] and SFC[column: DAICEL CHIRALPAK AD-H (250 mm×30 mm, 5 μm); mobile phase: [CO₂-EtOH (0.1% NH₃H₂O)]; B %: 35%, isocratic elution mode] to afford the title compound (1.00 g, 27% yield) as white solid; ¹H NMR (400 MHz, CHLOROFORM-d) δ=7.37-7.29 (m, 3H), 7.16-7.09 (m, 2H), 5.48 (s, 2H), 4.64 (br s, 2H), 3.61 (br s, 2H), 2.69-2.53 (m, 2H), 1.84 (br s, 2H), 1.41 (s, 9H); LCMS (ESI, M+1): m/z=329.2.

Step B. tert-butyl 4,6,7,8-tetrahydro-[1,2,3]triazolo[4,5-c]azepine-5(1H)-carboxylate: To a suspension of Pd/C (2.00 g, 10% purity) in ethanol (40 mL) were added tert-butyl 1-benzyl-4,6,7,8-tetrahydro-[1,2,3]triazolo[4,5-c]azepine-5(1H)-carboxylate (1.00 g, 1.0 equiv) and AcOH (18.3 mg, 0.1 equiv) under N₂ atmosphere. The reaction was degassed and purged with H₂ 3 times. The reaction was stirred at 80° C. for 4 hours under H₂ atmosphere (15 psi). The mixture was filtered and concentrated to afford the title compound (800 mg, crude) as white solid; LCMS (ESI, M+1): m/z=239.4.

Step C. tert-butyl 2-(dimethylcarbamoyl)-2,6,7,8-tetrahydro-[1,2,3]triazolo[4,5-c]azepine-5(4H)-carboxylate: To a solution of tert-butyl 4,6,7,8-tetrahydro-[1,2,3]triazolo[4,5-c]azepine-5(1H)-carboxylate (800 mg, 1.0 equiv) and 2,3,4,6,7,8,9,10-octahydropyrimido[1,2-a]azepine (2.04 g, 4.0 equiv) in dichloromethane (8.0 mL) was added dimethylcarbamic chloride (541 mg, 1.5 equiv) dropwise at 0° C. in 5 minutes. The reaction was stirred at 25° C. for 2 hours. The mixture was diluted with water (10 mL) and extracted with EtOAc (2×10 mL). The combined organic layers were washed with saturated brine (10 mL), dried over anhydrous sodium sulfate, concentrated and purified with reversed phase flash chromatography [C18, 0.1% formic acid condition] to afford the title compound (700 mg, 54% yield) as colorless oil.

Step D. N,N-dimethyl-5,6,7,8-tetrahydro-[1,2,3]triazolo[4,5-c]azepine-2(4H)-carboxamide: To a solution of tert-butyl 2-(dimethylcarbamoyl)-2,6,7,8-tetrahydro-[1,2,3]triazolo[4,5-c]azepine-5(4H)-carboxylate (400 mg, 1.0 equiv) in methanol (3 mL) was added HCl.MeOH (4 M, 8.0 mL) at 0° C. The reaction was stirred at 15° C. for 1 hour. The mixture was concentrated under vacuum. The residue was diluted with MeOH, neutralized with NaHCO₃(200 mg), filtered, and concentrated to afford the title compound (300 mg, crude) as yellow oil; LCMS (ESI, M+1): m/z=210.3.

Step E. tert-butyl (4-(6-chloro-4-(2-(dimethylcarbamoyl)-2,6,7,8-tetrahydro-[1,2,3]triazolo[4,5-c]azepin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (110 mg, 1.0 equiv) in DMSO (2.0 mL) were added TEA (68.9 mg, 4.0 equiv) and PyBOP (133 mg, 1.5 equiv). The reaction was stirred at 25° C. for 0.5 hours. N,N-dimethyl-5,6,7,8-tetrahydro-[1,2,3]triazolo[4,5-c]azepine-2(4H)-carboxamide (89.1 mg, 2.5 equiv) was added. The reaction was stirred at 25° C. for 2 hours. The mixture was diluted with water (10 mL) and extracted with EtOAc (2×10 mL). The combined organic layers were washed with brine (10 mL), dried over anhydrous sodium sulfate, concentrated, and purified with reversed phase flash chromatography [C18, 0.1% formic acid condition] to afford the title compound (35 mg, 24% yield) as white solid; LCMS (ESI, M+1): m/z=837.4.

Step F. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-[1,2,3]triazolo[4,5-c]azepine-2(4H)-carboxamide: To a solution of tert-butyl (4-(6-chloro-4-(2-(dimethylcarbamoyl)-2,6,7,8-tetrahydro-[1,2,3]triazolo[4,5-c]azepin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30.0 mg, 1.0 equiv) in dichloromethane (0.5 mL) was added TFA (1.0 mL) at 0° C. The reaction was stirred at 25° C. for 0.5 hours. The mixture was quenched with saturated NaHCO₃ solution (10 mL) at 0° C. and extracted with DCM (2×10 mL). The combined organic layers were washed with brine (10 mL), dried over anhydrous sodium sulfate, concentrated, and purified with prep-HPLC [column: Phenomenex luna C18 150×25 mm×10 μm; A: water (FA), B: ACN, B %: 30%-60% over 9 minutes] to afford the title compound (4.30 mg, 16% yield, 0.34 HCOOH salt) as white solid; ¹H NMR (400 MHz, METHANOL-d4) δ=8.03 (s, 1H), 7.20 (dd, J=5.2, 8.4 Hz, 1H), 7.11-6.96 (m, 1H), 5.40-5.28 (m, 1H), 5.14 (br s, 2H), 4.42-4.33 (m, 2H), 4.32-4.17 (m, 2H), 3.34 (br s, 3H), 3.21 (br s, 6H), 3.11-3.04 (m, 1H), 2.99 (br dd, J=3.6, 6.8 Hz, 2H), 2.42-2.14 (m, 5H), 2.07-1.90 (m, 3H); LCMS (ESI, M+1): m/z=737.2.

### Example 169

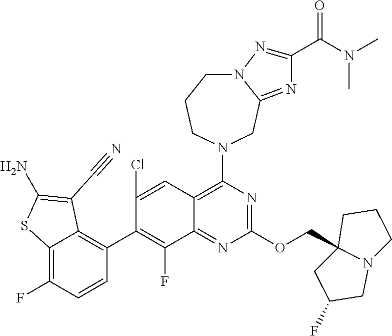 [SMILES: CN(C)C(=O)c1nc2n(n1)CCCN(c1nc(OC[C@@]34CCCN3C[C@H](F)C4)nc3c(F)c(-c4ccc(F)c5sc(N)c(C#N)c45)c(Cl)cc13)C2]

### 8-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-6,7,8,9-tetrahydro-5H-[1,2,4]triazolo[1,5-a][1,4]diazepine-2-carboxamide

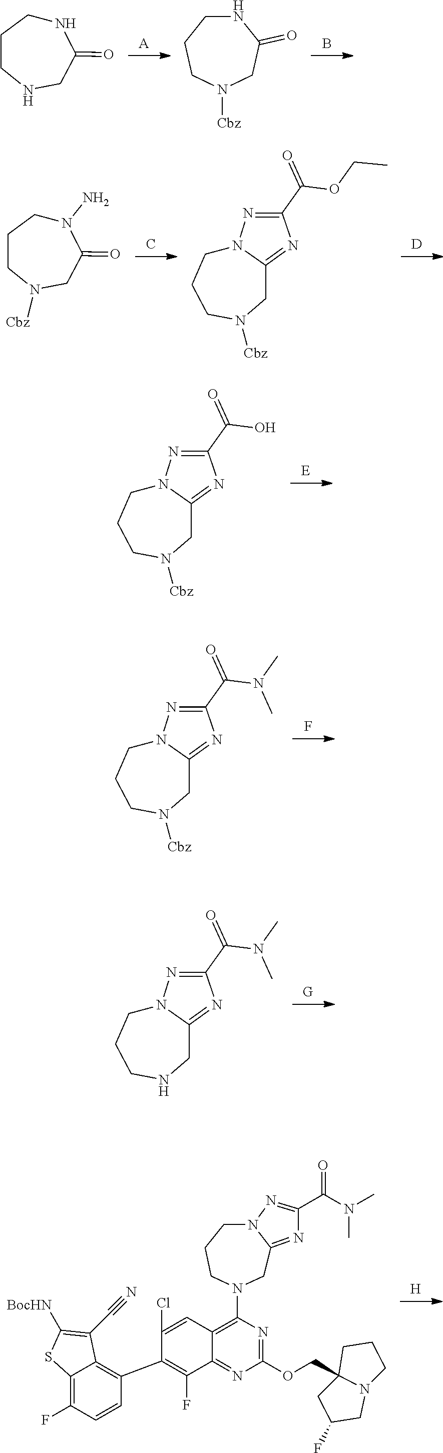

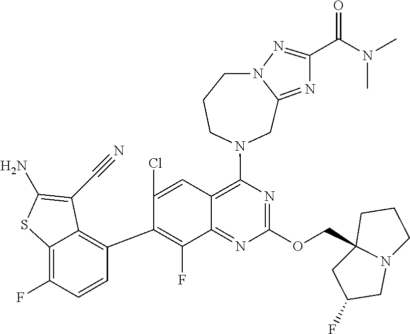 [SMILES: CN(C)C(=O)c1nc2n(n1)CCCN(c1nc(OC[C@@]34CCCN3C[C@H](F)C4)nc3c(F)c(-c4ccc(F)c5sc(N)c(C#N)c45)c(Cl)cc13)C2]

Step A. benzyl 3-oxo-1,4-diazepane-1-carboxylate: To a solution of 1,4-diazepan-2-one (4.0 g, 1.0 equiv, HCl) and NaHCO₃(5.58 g, 2.5 equiv) in H₂O (20 mL) and THE (20 mL) was added CbzCl (4.76 g, 1.05 equiv). The reaction was stirred at 20° C. for 1 hour. The mixture was diluted with water (30 mL) and extracted with EtOAc (3×20 mL). The combined organic layers were washed with brine (50 mL), dried over anhydrous sodium sulfate, concentrated and purified with column chromatography [SiO₂, Dichloromethane/Methanol=10/1 to 3/1] to afford the title compound (5.30 g, 80% yield) as white solid; ¹H NMR (400 MHz, CHLOROFORM-d) δ=7.44-7.29 (m, 5H), 6.46-6.04 (m, 1H), 5.18 (s, 2H), 4.30-4.06 (m, 2H), 3.68-3.66 (m, 2H), 3.30 (br s, 2H), 1.91 (br s, 2H); LCMS (ESI, M+1): m/z=249.2.

Step B. benzyl 4-amino-3-oxo-1,4-diazepane-1-carboxylate: To a solution of benzyl 3-oxo-1,4-diazepane-1-carboxylate (5.30 g, 1.0 equiv) and t-BuOK (1 M, 53.4 mL, 2.5 equiv) in DMF (50 mL) was added hydroxylamine-O-sulfonic acid (4.83 g, 2.0 equiv) at 0° C. The reaction was stirred at 0° C. for 1 hour. The mixture was diluted with water (100 mL) and extracted with EtOAc (3×50 mL). The combined organic layers were washed with brine (200 mL), dried over anhydrous sodium sulfate, and concentrated to afford the title compound (5.00 g, crude) as yellow oil; LCMS (ESI, M+1): m/z=264.2.

Step C. 8-benzyl 2-ethyl 6,7-dihydro-5H-[1,2,4]triazolo[1,5-a][1,4]diazepine-2,8(9H)-dicarboxylate: To a solution of benzyl 4-amino-3-oxo-1,4-diazepane-1-carboxylate (1.00 g, 50% purity, 1.0 equiv) in EtOH (10 mL) were added ethyl 2-ethoxy-2-imino-acetate (1.10 g, 2.0 equiv) and AcOH (798 mg, 3.5 equiv) at 20° C. The reaction was stirred at 78° C. for 12 hours. The mixture was concentrated. The residue was dissolved in water (20 mL) and extracted with EtOAc (4×20 mL). The combined organic layers were washed with brine (80 mL), dried over anhydrous sodium sulfate, concentrated, and purified with reversed phase flash chromatography [C18, 0.1% formic acid condition] to afford the title compound (500 mg, 30% yield) as yellow oil; LCMS (ESI, M+1): m/z=345.2.

Step D. 8-((benzyloxy)carbonyl)-6,7,8,9-tetrahydro-5H-[1,2,4]triazolo[1,5-a][1,4]diazepine-2-carboxylic acid: To a solution of 8-benzyl 2-ethyl 6,7-dihydro-5H-[1,2,4]triazolo[1,5-a][1,4]diazepine-2,8(9H)-dicarboxylate (650 mg, 1.0 equiv) in EtOH (4 mL) was added NaOH (1 M, 3.78 mL, 2.0 equiv) at 0° C. The reaction was stirred at 25° C. for 1 hour. The pH of the mixture was adjusted to 3 with HCl (3 mL, 2 M) at 0° C. The mixture was extracted with EtOAc (3×5 mL). The combined organic layers were dried over anhydrous sodium sulfate and concentrated to afford the title compound (500 mg, crude) as white solid; LCMS (ESI, M+1): m/z=317.1.

Step E. benzyl 2-(dimethylcarbamoyl)-6,7-dihydro-5H-[1,2,4]triazolo[1,5-a][1,4]diazepine-8(9H)-carboxylate: To a mixture of 8-((benzyloxy)carbonyl)-6,7,8,9-tetrahydro-5H-[1,2,4]triazolo[1,5-a][1,4]diazepine-2-carboxylic acid (500 mg, 1.0 equiv), EDCI (606 mg, 2 equiv), HOBt (256 mg, 1.2 equiv) and TEA (1.28 g, 8.0 equiv) in DMF (5 mL) was added dimethylamine (2 M, 3.95 mL, 5.0 equiv). The reaction was stirred at 40° C. for 12 hours. The mixture was diluted with water (10 mL) and extracted with EtOAc (3×10 mL). The combined organic layers were washed with brine (30 mL), dried over anhydrous sodium sulfate, concentrated, and purified with reversed phase flash chromatography [C18, 0.1% formic acid condition] to afford the title compound (200 mg, 36% yield) as yellow oil; LCMS (ESI, M+1): m/z=344.2

Step F. N,N-dimethyl-6,7,8,9-tetrahydro-5H-[1,2,4]triazolo[1,5-a][1,4]diazepine-2-carboxamide: To a solution of benzyl 2-(dimethylcarbamoyl)-6,7-dihydro-5H-[1,2,4]triazolo[1,5-a][1,4]diazepine-8(9H)-carboxylate (200 mg, 1.0 equiv) in MeOH (2 mL) was added Pd/C (100 mg, 10% purity) under N₂ atmosphere. The suspension was degassed and purged with H₂ for 3 times. The reaction was stirred at 20° C. for 1 hour under H₂ (15 Psi or atm). The mixture was filtered and concentrated under vacuum to afford the title compound (120 mg, crude) as white solid; LCMS (ESI, M+1): m/z=210.1

Step G. tert-butyl (4-(6-chloro-4-(2-(dimethylcarbamoyl)-6,7-dihydro-5H-[1,2,4]triazolo[1,5-a][1,4]diazepin-8(9H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (110 mg, 1.0 equiv) and PyBOP (133 mg, 1.5 equiv) in DMSO (1.0 mL) was added and TEA (51.7 mg, 3 equiv). The reaction was stirred at 30° C. for 1 hour and N,N-dimethyl-6,7,8,9-tetrahydro-5H-[1,2,4]triazolo[1,5-a][1,4]diazepine-2-carboxamide (71.3 mg, 2.0 equiv) was added. The reaction was stirred at 30° C. for 12 hours. The mixture was filtered and purified with reversed phase flash chromatography [C18, 0.1% formic acid condition] to afford the title compound (110 mg, 77% yield) as white solid; LCMS (ESI, M+1): m/z=837.3.

Step H. 8-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-6,7,8,9-tetrahydro-5H-[1,2,4]triazolo[1,5-a][1,4]diazepine-2-carboxamide: To a solution of tert-butyl (4-(6-chloro-4-(2-(dimethylcarbamoyl)-6,7-dihydro-5H-[1,2,4]triazolo[1,5-a][1,4]diazepin-8(9H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (100 mg, 1.0 equiv) in DCM (1.0 mL) was added TFA (2.0 mL). The reaction was stirred at 20° C. for 0.5 hours. The pH of the mixture was adjusted to 8 with saturated NaHCO₃ aqueous (5.0 mL) at 0° C. The mixture was extracted with EtOAc (3×5 mL). The combined organic layers were washed with brine (20 mL), dried over anhydrous sodium sulfate, concentrated and purified with prep-HPLC [column: Waters Xbridge 150×25 mm×5 μm; A: water (ammonia hydroxide v/v), B: ACN, 34%-64% over 9 min] and lyophilized to afford the title compound (12.2 mg, 14% yield) as white solid; H NMR (400 MHz, METHANOL-d₄) δ=8.04 (d, J=1.2 Hz, 1H), 7.22-7.19 (m, 1H), 7.09-6.98 (m, 1H), 5.38-5.21 (m, 1H), 5.20-5.09 (m, 2H), 4.48-4.27 (m, 4H), 4.19-4.07 (m, 2H), 3.34 (s, 4H), 3.27-3.17 (m, 2H), 3.12 (s, 3H), 3.04-2.95 (m, 1H), 2.51-2.38 (m, 2H), 2.37-2.05 (m, 3H), 2.01-1.84 (m, 3H); LCMS (ESI, M+1): m/z=737.3.

### Example 170

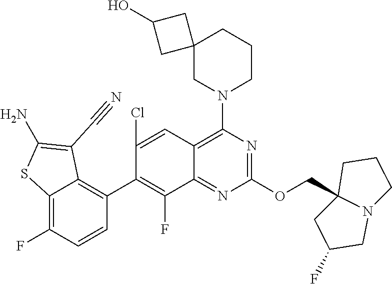 [SMILES: N#Cc1c(N)sc2c(F)ccc(-c3c(Cl)cc4c(N5CCCC6(CC(O)C6)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-7-fluorobenzo[b]thiophene-3-carbonitrile

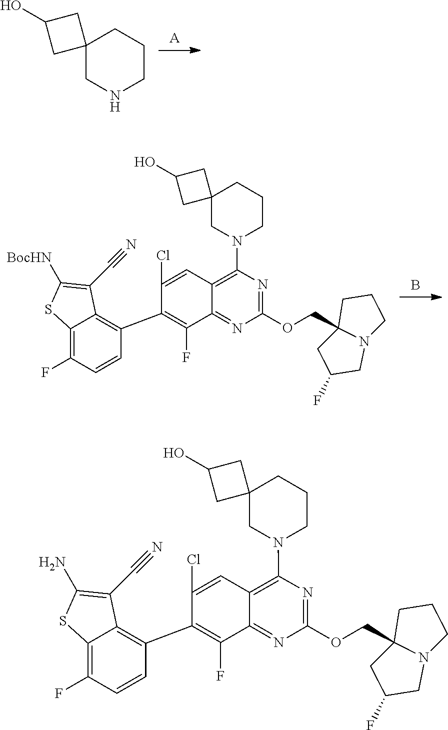

Step A. tert-butyl (4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) in DMSO (1.0 mL) were added PyBOP (121 mg, 1.5 equiv) and TEA (94 mg, 6.0 equiv). The reaction was stirred at 30° C. for 20 minutes and 6-azaspiro[3.5]nonan-2-ol (44 mg, 2.0 equiv) was added. The reaction was stirred at 30° C. for 24 hours. The mixture was filtered and purified with reversed phase chromatography [0.1% FA condition] to afford the title compound (40 mg, 32% yield) as yellow solid; LCMS (ESI, M+1): m/z=769.5.

Step B. 2-amino-4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(2-hydroxy-6-azaspiro[3.5]nonan-6-yl)quinazolin-7-yl)-3-cyano-7-fluorobenzo[b]thiophen-2-yl)carbamate (35 mg, 1.0 equiv) in DCM (2.0 mL) was added TFA (4.0 mL) at 0° C. The reaction was stirred at 20° C. for 1 hour. The mixture was concentrated. The residue was dissolved in EtOAc (3.0 mL) and neutralized with saturated NaHCO₃ aqueous (5.0 mL) at 0° C. The mixture was extracted with EtOAc (2×5 mL). The combined organic layers were wash with brine (5.0 mL), dried over anhydrous sodium sulfate, concentrated and purified with prep-HPLC [column: Waters Xbridge 150×25 mm×5 μm; mobile phase: water (ammonia hydroxide v/v)−ACN; gradient: 45%-75% over 9 minutes] to afford the title compound (17.7 mg, 58% yield) as white solid; ¹H NMR (400 MHz, CD₃OD) δ=7.94 (d, J=10.4 Hz, 1H), 7.29-7.18 (m, 1H), 7.13-7.00 (m, 1H), 5.69-5.44 (m, 1H), 4.79-4.55 (m, 2H), 4.27 (t, J=7.2 Hz, 1H), 4.11-3.74 (m, 7H), 3.54-3.40 (m, 1H), 2.79-2.08 (m, 8H), 1.99-1.55 (m, 6H); LCMS (ESI, M+1): m/z=669.5.

### Example 171

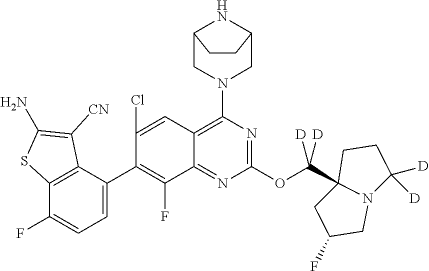 [SMILES: [2H]C1([2H])CC[C@@]2(C([2H])([2H])Oc3nc(N4CC5CCC(C4)N5)c4cc(Cl)c(-c5ccc(F)c6sc(N)c(C#N)c56)c(F)c4n3)C[C@@H](F)CN12]

### 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl-5,5-d2)methoxy-d2)quinazolin-7-yl)-2-amino-7-fluorobenzo[b]thiophene-3-carbonitrile

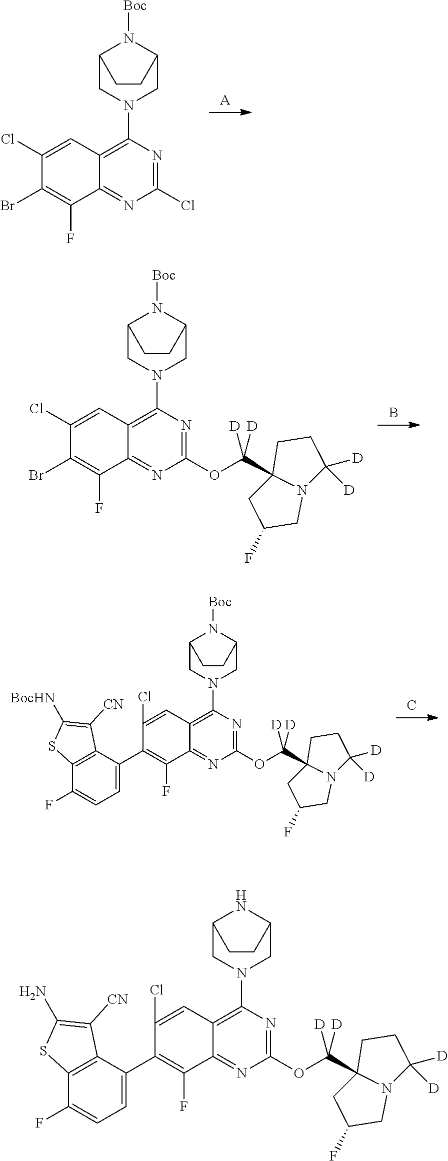

Step A. tert-butyl (1R,5S)-3-(7-bromo-6-chloro-8-fluoro-2-(((2R,7aS)-2-fluorotetrahydro-1H-pyrrolizin-7a(5H)-yl-5,5-d2)methoxy-d2)quinazolin-4-yl)-3,8-diazabicyclo[3.2.1]octane-8-carboxylate: To a mixture of tert-butyl (1R,5S)-3-(7-bromo-2,6-dichloro-8-fluoroquinazolin-4-yl)-3,8-diazabicyclo[3.2.1]octane-8-carboxylate (500 mg, 1.0 equiv), ((2R,7aS)-2-fluorotetrahydro-1H-pyrrolizin-7a(5H)-yl-5,5-d2)methan-d2-ol (225 mg, 1.4 equiv) and Cs₂CO₃ (965 mg, 3.0 equiv) in DMF (5 mL) THE (5 mL) was added 1,4-diazabicyclo[2.2.2]octane (110 mg, 1.0 equiv). The reaction was stirred at 50° C. for 3 hours. The mixture was diluted with H₂O (20 ml) and extracted with EtOAc (2×30 ml). The combined organic layers were washed with brine (5 mL), dried over anhydrous sodium sulfate, concentrated and purified with reversed phase flash chromatography [C18, 0.1% formic acid condition] to afford title compound (130 mg, 17% yield) as a white solid; LCMS (ESI, M+1, M+3): m/z=632.3, 634.3.

Step B. tert-butyl (1R,5S)-3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl-5,5-d2)methoxy-d2)quinazolin-4-yl)-3,8-diazabicyclo[3.2.1]octane-8-carboxylate: To a mixture of tert-butyl (1R,5S)-3-(7-bromo-6-chloro-8-fluoro-2-(((2R,7aS)-2-fluorotetrahydro-1H-pyrrolizin-7a(5H)-yl-5,5-d2)methoxy-d2)quinazolin-4-yl)-3,8-diazabicyclo[3.2.1]octane-8-carboxylate (130 mg, 1.0 equiv), tert-butyl N-[3-cyano-4-(5,5-dimethyl-1,3,2-dioxaborinan-2-yl)-7-fluoro-benzothiophen-2-yl]carbamate (166 mg, 2.0 equiv) and Cs₂CO₃ (200 mg, 3 equiv) in toluene (2 mL) was added Pd(DPEphos)Cl₂ (14.1 mg, 0.1 equiv). The reaction was degassed and purged with nitrogen 3 times. The reaction was stirred at 90° C. for 1 hour. The mixture was diluted with water (10 mL) and extracted with EtOAc (2×15 mL). The combined organic layers were concentrated and purified with reversed phase flash chromatography [C18, 0.1% formic acid condition] to afford the title compound (55 mg, 24% yield) as a white solid; LCMS (ESI, M+1): m/z=844.4.

Step C. 4-(4-((1R,5S)-3,8-diazabicyclo[3.2.1]octan-3-yl)-6-chloro-8-fluoro-2-(((2R,7aS)-2-fluorotetrahydro-1H-pyrrolizin-7a(5H)-yl-5,5-d2)methoxy-d2)quinazolin-7-yl)-2-amino-7-fluorobenzo[b]thiophene-3-carbonitrile: To a solution of tert-butyl (1R,5S)-3-(7-(2-((tert-butoxycarbonyl)amino)-3-cyano-7-fluorobenzo[b]thiophen-4-yl)-6-chloro-8-fluoro-2-(((2R,7aS)-2-fluorotetrahydro-1H-pyrrolizin-7a(5H)-yl-5,5-d2)methoxy-d2)quinazolin-4-yl)-3,8-diazabicyclo[3.2.1]octane-8-carboxylate (50 mg, 1.0 equiv) in DCM (1 mL) was added TFA (3.07 g, 454 equiv) at 0° C. The reaction was stirred at 25° C. for 0.5 hours. The mixture was basified with NaHCO₃ aqueous (10 ml) and extracted with EtOAc (2×10 mL). The combined organic layers were washed with brine (10 mL), dried over anhydrous sodium sulfate, concentrated and purified with prep-HPLC [column: Phenomenex luna 150×25 mm×5 μm; A: water (FA), B: ACN; B %: 8%-28% over 10 min] to afford the title compound (3.05 mg, 6.7% yield, 0.57 HCOOH) as a white solid; 1H NMR (400 MHz, METHANOL-d₄) δ=7.88 (s, 1H), 7.22-7.18 (m, 1H), 7.06-7.01 (t, J=8.8 Hz, 1H), 5.43 (d, J=52.4 Hz,1H), 4.58-4.48 (m, 2H), 3.94-3.74 (m, 1H), 3.72-3.67 (m, 2H), 3.67-3.48 (m, 1H), 3.36-3.31 (m, 2H), 2.65-2.64 (m, 1H), 2.50-2.26 (m, 2H), 2.25-2.15 (m, 1H), 2.10-1.95 (m, 3H), 1.89 (s, 3H); LCMS (ESI, M+1): m/z=644.3.

### Examples 172 and 173

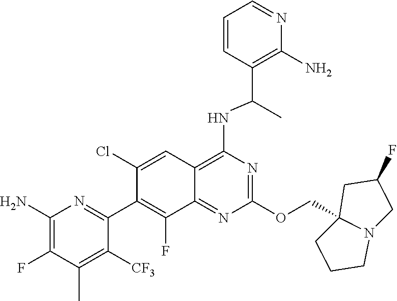 [SMILES: Cc1c(F)c(N)nc(-c2c(Cl)cc3c(NC(C)c4cccnc4N)nc(OC[C@@]45CCCN4C[C@H](F)C5)nc3c2F)c1C(F)(F)F]

### 7-(6-amino-5-fluoro-4-methyl-3-(trifluoromethyl)pyridin-2-yl)-N-(1-(2-aminopyridin-3-yl)ethyl)-6-chloro-8-fluoro-2-(((2R,7aS)-2-fluorotetrahydro-1H-pyrrolizin-7a(5H)-yl)methoxy)quinazolin-4-amine (isomer 1)

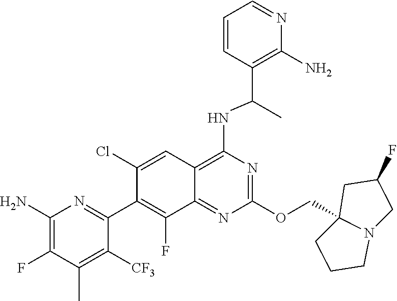 [SMILES: Cc1c(F)c(N)nc(-c2c(Cl)cc3c(NC(C)c4cccnc4N)nc(OC[C@@]45CCCN4C[C@H](F)C5)nc3c2F)c1C(F)(F)F]

### 7-(6-amino-5-fluoro-4-methyl-3-(trifluoromethyl)pyridin-2-yl)-N-(1-(2-aminopyridin-3-yl)ethyl)-6-chloro-8-fluoro-2-(((2R,7aS)-2-fluorotetrahydro-1H-pyrrolizin-7a(5H)-yl)methoxy)quinazolin-4-amine (isomer 2)

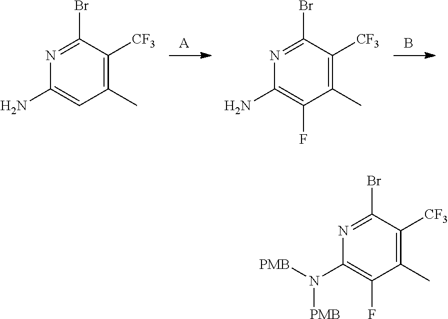

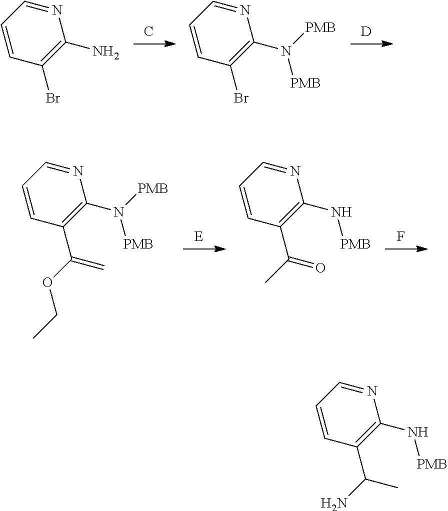

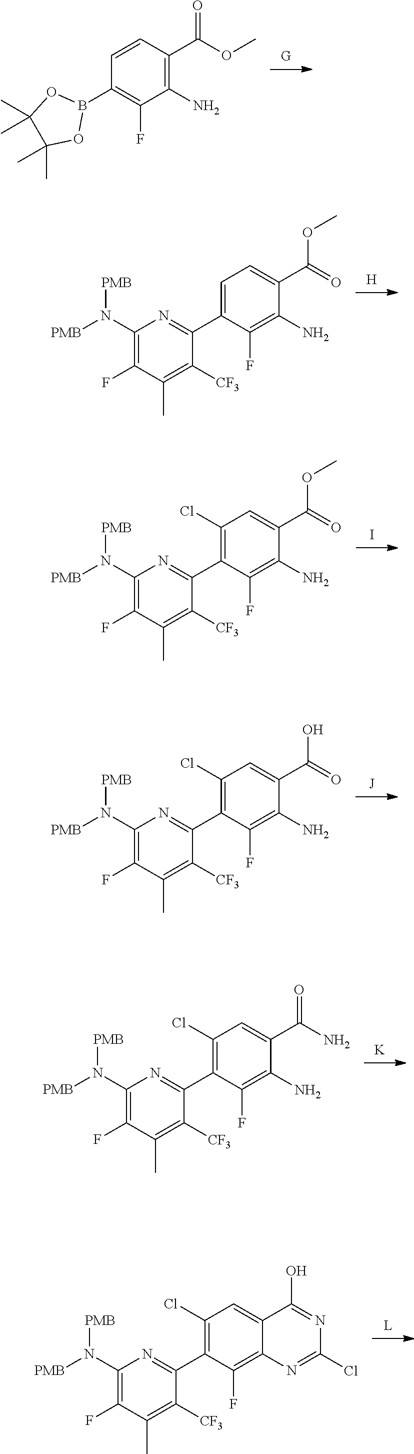

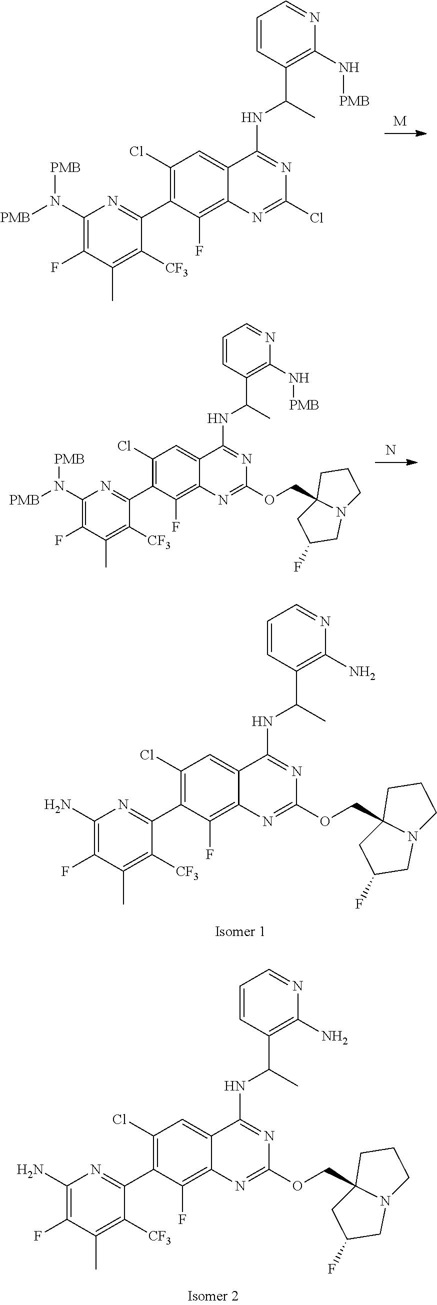

Step A. 6-bromo-3-fluoro-4-methyl-5-(trifluoromethyl)pyridin-2-amine: To a mixture of 6-bromo-4-methyl-5-(trifluoromethyl)pyridin-2-amine (20.0 g, 1.0 equiv) in ACN (400 mL) was added 1-(chloromethyl)-4-fluoro-1,4-diazoniabicyclo[2.2.2]octane; ditetrafluoroborate (55.6 g, 2.0 equiv). The reaction was stirred at 25° C. for 12 hours. The mixture was diluted with H₂O (600 mL) and extracted with ethyl acetate (3×400 mL). The combined organic layers were washed with brine (3×400 mL), dried over anhydrous sodium sulfate and concentrated to afford the title compound (7.00 g, crude) as brown solid.

Step B. 6-bromo-3-fluoro-N,N-bis(4-methoxybenzyl)-4-methyl-5-(trifluoromethyl)pyridin-2-amine: To a mixture of 6-bromo-3-fluoro-4-methyl-5-(trifluoromethyl)pyridin-2-amine (5.40 g, 1.0 equiv) in THE (100 mL) was added NaH (2.37 g, 60.0% purity, 3.0 equiv) portion-wise at 25° C. The reaction was stirred for 1 hour. PMBCl (9.29 g, 3.0 equiv) was added. The reaction was stirred at 25° C. for 2 hours. The mixture was quenched by addition of saturated NH₄Cl aqueous (200 mL) at 0˜5° C. and the mixture was stirred for 30 mins. The mixture was extracted with ethyl acetate (3×100 mL). The combined organic layers were washed with brine (3×100 mL), dried over anhydrous sodium sulfate, concentrated and purified by pre-HPLC (column: Phenomenex luna c18 250 mm*100 mm*10 um; mobile phase: [water (TFA)−ACN]; gradient: 70%-100% B over 24 min) to afford the title compound (8.70 g, 86% yield) as brown solid.

Step C. 3-bromo-N,N-bis(4-methoxybenzyl)pyridin-2-amine: To a solution of 3-bromopyridin-2-amine (30.0 g, 1.0 equiv) in THE (300 mL) was added NaH (20.8 g, 60.0% purity, 3.0 equiv) at 15° C. The reaction was stirred for 1 hour. PMBCl (81.5 g, 70.6 mL, 3.0 equiv) was added drop-wise at 0˜ 5° C. into the mixture. The reaction was stirred at 25° C. for 15 hours. The mixture was poured into saturated aq.NH₄Cl (200 mL) at 0˜ 5° C. and stirred for 30 mins. The mixture was extracted with ethyl acetate (3×300 mL). The combined organic layers were washed with brine (3×300 mL), dried over anhydrous sodium sulfate, concentrated and purified by column chromatography (SiO₂, petroleum ether/ethyl acetate=100/1 to 10/1) to give the crude product. The crude product was triturated with MTBE: EA=1:4 (3.00 V, 120 mL) at 20° C. for 1 hour to afford the title compound (55.3 g, 75% yield) as white solid; ¹H NMR: (400 MHz, DMSO d6) δ 8.18-8.17 (m, 1H), 7.99-7.96 (m, 1H), 7.20-7.18 (m, 4H), 6.90-6.88 (m, 1H), 6.87-6.82 (m, 4H), 4.32 (s, 4H), 3.69 (s, 6H); LCMS (ESI, M+1): m/z=413.0.

Step D. 3-(1-ethoxyvinyl)-N,N-bis(4-methoxybenzyl)pyridin-2-amine: To a solution of 3-bromo-N,N-bis(4-methoxybenzyl)pyridin-2-amine (45.0 g, 1.0 equiv) in DMF (450 mL) were added tributyl(1-ethoxyvinyl)stannane (115 g, 2.9 equiv) and Pd(PPh₃)₂Cl₂ (7.64 g, 0.1 equiv). The reaction was degassed and purged with nitrogen 3 times. The reaction was stirred at 80° C. for 12 hours under CO. The mixture was diluted with H₂O (500 mL) and extracted with ethyl acetate (3×500 mL), the combined organic phase was washed with 10.0% aqueous of KF (500 mL), brine (3×500 mL), dried over anhydrous sodium sulfate and concentrated to afford the title compound (44.0 g, crude) as a black brown oil; LCMS (ESI, M+1): m/z=405.2.

Step E. 1-(2-((4-methoxybenzyl)amino)pyridin-3-yl)ethan-1-one: To a solution of 3-(1-ethoxyvinyl)-N,N-bis(4-methoxybenzyl)pyridin-2-amine (44.0 g, 1.0 equiv) in THE (400 mL) was added HCl (2 M, 272 mL, 5.00 equiv). The reaction was stirred at 20° C. for 2 hours. The mixture was diluted with H₂O (400 mL) and extracted with ethyl acetate (3×400 mL). The combined organic layers were washed with saturated aqueous of NaHCO₃(400 mL) and brine (3×400 mL), dried over anhydrous sodium sulfate, concentrated and purified by prep-HPLC (TFA condition) to afford the title compound (16.0 g, 32% yield) as yellow solid; LCMS (ESI, M+1): m/z=257.1.

Step F. 3-(1-aminoethyl)-N-(4-methoxybenzyl)pyridin-2-amine: A mixture of 1-(2-((4-methoxybenzyl)amino)pyridin-3-yl)ethan-1-one (3.40 g, 1.0 equiv), NH₄C1 (7.10 g, 10.0 equiv) and tetraethoxytitanium (6.05 g, 2.0 equiv) in THE (34.0 mL) was stirred at 70° C. for 2 hours under N₂ atmosphere. Then the reaction was cooled to 25° C. and added NaBH₃CN (1.67 g, 26.5 mmol, 2.0 equiv). The reaction was stirred at 25° C. for 1 hour. The mixture was diluted with H₂O (50.0 mL) and extracted with ethyl acetate (3×50.0 mL). The combined organic layers were washed with brine (2×50.0 mL), dried over anhydrous sodium sulfate, concentrated and purified by reversed-phase HPLC (0.1% FA condition) to afford the title compound (480 mg, 14% yield) as yellow solid; LCMS (ESI, M+1): m/z=258.1.

Step G. methyl 2-amino-4-(6-(bis(4-methoxybenzyl)amino)-5-fluoro-4-methyl-3-(trifluoromethyl)pyridin-2-yl)-3-fluorobenzoate: To a mixture of methyl 2-amino-3-fluoro-4-(4,4,5,5-tetramethyl-1,3,2-dioxaborolan-2-yl)benzoate (2.46 g, 1.00 equiv) and 6-bromo-3-fluoro-N,N-bis(4-methoxybenzyl)-4-methyl-5-(trifluoromethyl)pyridin-2-amine (5.05 g, 1.18 equiv) in ACN (10.0 mL) were added K₃PO₄ (5.31 g, 3.00 equiv) and Pd(dppf)Cl₂ (762 mg, 0.125 equiv). The reaction was degassed and purged with nitrogen 3 times. The mixture was stirred at 70° C. for 2 hours. The mixture was concentrated and purified by column chromatography (SiO₂, petroleum ether/ethyl acetate=50/1 to 10/1, petroleum ether/ethyl acetate=5/1, Rf=0.40) to afford the title compound (3.93 g, 75% yield) as yellow solid; LCMS (ESI, M+1): m/z=602.3.

Step H. methyl 2-amino-4-(6-(bis(4-methoxybenzyl)amino)-5-fluoro-4-methyl-3-(trifluoromethyl)pyridin-2-yl)-5-chloro-3-fluorobenzoate: To a solution of methyl 2-amino-4-(6-(bis(4-methoxybenzyl)amino)-5-fluoro-4-methyl-3-(trifluoromethyl)pyridin-2-yl)-3-fluorobenzoate (3.93 g, 1.0 equiv) in DMF (39.3 mL) was added NCS (1.08 g, 1.3 equiv). The reaction was degassed and purged with nitrogen 3 times. The reaction was stirred at 90° C. for 1 hour. The mixture was cooled to 25° C. and poured into 200 mL of water, filtered. The solids were collected and dried to afford the title compound (4.12 g, crude) as yellow solid; LCMS (ESI, M+1): m/z=636.2.

Step I. 2-amino-4-(6-(bis(4-methoxybenzyl)amino)-5-fluoro-4-methyl-3-(trifluoromethyl)pyridin-2-yl)-5-chloro-3-fluorobenzoic acid: To a mixture of methyl 2-amino-4-(6-(bis(4-methoxybenzyl)amino)-5-fluoro-4-methyl-3-(trifluoromethyl)pyridin-2-yl)-5-chloro-3-fluorobenzoate (4.12 g, 1.0 equiv) in THE (41.2 mL) was added a solution of LiOH.H₂O (652 mg, 3.0 equiv) in H₂O (13.7 mL). The reaction was stirred at 50° C. for 3 hours. The mixture was cooled to 25° C. The pH of the mixture was adjusted to 4 with citric acid. The mixture was diluted with H₂O (50.0 mL) and extracted with ethyl acetate (3×50 mL). The combined organic layers were washed with brine (3×50 mL), dried over anhydrous sodium sulfate and concentrated to afford the title compound (4.30 g, crude) as brown oil; LCMS (ESI, M+1): m/z=622.3.

Step J. 2-amino-4-(6-(bis(4-methoxybenzyl)amino)-5-fluoro-4-methyl-3-(trifluoromethyl)pyridin-2-yl)-5-chloro-3-fluorobenzamide: To a mixture of 2-amino-4-(6-(bis(4-methoxybenzyl)amino)-5-fluoro-4-methyl-3-(trifluoromethyl)pyridin-2-yl)-5-chloro-3-fluorobenzoic acid (4.30 g, 1.0 equiv) in DMF (43.0 mL) were added DIPEA (2.12 g, 3.0 equiv), NH₄C1 (878 mg, 3.0 equiv) and HATU (4.16 g, 2.0 equiv). The reaction was stirred at 25° C. for 2 hours. The mixture was cooled to 25° C. and poured into 215 mL of water. The solids were collected and purified by column chromatography (SiO₂, petroleum ether/ethyl acetate=20/1 to 1/1) to afford the title compound (3.56 g, 95% yield) as yellow solid; LCMS (ESI, M+1): m/z=621.2.

Step K. 7-(6-(bis(4-methoxybenzyl)amino)-5-fluoro-4-methyl-3-(trifluoromethyl)pyridin-2-yl)-2,6-dichloro-8-fluoroquinazolin-4-ol: A mixture of 2-amino-4-(6-(bis(4-methoxybenzyl)amino)-5-fluoro-4-methyl-3-(trifluoromethyl)pyridin-2-yl)-5-chloro-3-fluorobenzamide (3.56 g, 1.0 equiv) and thiocarbonyl dichloride (1.98 g, 3.0 equiv) in dioxane (35.6 mL) was stirred at 80° C. for 0.5 hour under N₂ atmosphere. The mixture was added to aq.NaHCO₃(50.0 mL) and extracted with ethyl acetate (3×50.0 mL). The combined organic layers were washed with brine (3×50.0 mL), dried over anhydrous sodium sulfate, concentrated and purified by reversed-phase HPLC (0.1% FA condition) to afford the title compound (1.45 g, 36% yield) as white solid; LCMS (ESI, M+1): m/z=665.1.

Step L. 7-(6-(bis(4-methoxybenzyl)amino)-5-fluoro-4-methyl-3-(trifluoromethyl)pyridin-2-yl)-2,6-dichloro-8-fluoro-N-(1-(2-((4-methoxybenzyl)amino)pyridin-3-yl)ethyl)quinazolin-4-amine: To a mixture of 7-(6-(bis(4-methoxybenzyl)amino)-5-fluoro-4-methyl-3-(trifluoromethyl)pyridin-2-yl)-2,6-dichloro-8-fluoroquinazolin-4-ol (800 mg, 1.0 equiv) and 3-(1-aminoethyl)-N-(4-methoxybenzyl)pyridin-2-amine (371 mg, 1.2 equiv) in DCM (8.00 mL) were added BOPCl (918 mg, 3.0 equiv) and DIPEA (1.55 g, 10.0 equiv). The reaction was stirred at 25° C. for 16 hours. The mixture was diluted with H₂O (20.0 mL) and extracted with ethyl acetate (3×20.0 mL). The combined organic layers were washed with brine (3×20.0 mL), dried over dried over anhydrous sodium sulfate, concentrated and purified by reversed-phase HPLC (column: Phenomenex luna C18 150*40 mm*15 um; mobile phase: [water (TFA)−ACN]; gradient: 52%-82% B over 15 min) to afford the title compound (666 mg, 59.5% yield) as yellow solid; LCMS (ESI, M+1): m/z=904.3.

Step M. 7-(6-(bis(4-methoxybenzyl)amino)-5-fluoro-4-methyl-3-(trifluoromethyl)pyridin-2-yl)-6-chloro-8-fluoro-2-(((2R,7aS)-2-fluorohexahydro-1H-pyrrolizin-7a-yl)methoxy)-N-(1-(2-((4-methoxybenzyl)amino)pyridin-3-yl)ethyl)quinazolin-4-amine: To a solution of 7-(6-(bis(4-methoxybenzyl)amino)-5-fluoro-4-methyl-3-(trifluoromethyl)pyridin-2-yl)-2,6-dichloro-8-fluoro-N-(1-(2-((4-methoxybenzyl)amino)pyridin-3-yl)ethyl)quinazolin-4-amine (351 mg, 3.00 equiv) and ((2S,3aR)-2-fluorohexahydropentalen-3a(1H)-yl)methanol (666 mg, 1.0 equiv) in toluene (10.0 mL) were added t-BuONa (212 mg, 3.0 equiv), Pd₂(dba)₃ (33.7 mg, 0.05 equiv) and BINAP (45.8 mg, 0.10 equiv). The reaction was degassed and purged with nitrogen 3 times. The reaction was stirred at 110° C. for 1 hour. The mixture was dliuted with H₂O (10.0 mL) and extracted with ethyl acetate (3×10.0 mL). The combined organic layers were washed with brine (2×10.0 mL), dried over anhydrous sodium sulfate, concentrated and purified by reversed-phase HPLC (column: Phenomenex luna C18 150*25 mm*10 um; mobile phase: [water (TFA)−ACN]; gradient: 42%-72% B over min) to afford the title compound (450 mg, 59% yield) as yellow solid; LCMS (ESI, M+1): m/z=1027.4.

Step N. 7-(6-amino-5-fluoro-4-methyl-3-(trifluoromethyl)pyridin-2-yl)-N-(1-(2-aminopyridin-3-yl)ethyl)-6-chloro-8-fluoro-2-(((2R,7aS)-2-fluorohexahydro-1H-pyrrolizin-7a-yl)methoxy)quinazolin-4-amine: A mixture of 7-(6-(bis(4-methoxybenzyl)amino)-5-fluoro-4-methyl-3-(trifluoromethyl)pyridin-2-yl)-6-chloro-8-fluoro-2-(((2R,7aS)-2-fluorohexahydro-1H-pyrrolizin-7a-yl)methoxy)-N-(1-(2-((4-methoxybenzyl)amino)pyridin-3-yl)ethyl)quinazolin-4-amine (100 mg, 1.0 equiv) and TFA (3.00 mL) was stirred at 50° C. for 0.5 hours under N₂ atmosphere. The mixture was diluted with H₂O (2.00 mL) and extracted with ethyl acetate (3×2.00 mL). The combined organic phase was washed with brine (2×2.00 mL), dried over anhydrous sodium sulfate, concentrated and purified by reversed-phase HPLC (column: Waters Xbridge 150*25 mm*5 um; mobile phase: [water (NH₄HCO₃)−ACN]; gradient: 44%-74% B over 9 min) to afford two peaks.

Peak 1: Example 172 (22.6 mg, 17% yield) as white solid; ¹H NMR: (400 MHz, DMSO d6) δ 8.73 (d, J=6.4 Hz, 1H), 8.46 (s, 1H), 7.85 (d, J=3.6 Hz, 1H), 7.49-7.46 (m, 1H), 7.23 (s, 2H), 6.57-6.53 (m, 1H), 5.90 (s, 2H), 5.45-5.42 (m, 1H), 5.40-5.28 (m, 1H), 4.07-3.95 (m, 2H), 3.28-2.99 (m, 3H), 2.95-2.75 (m, 1H), 2.34 (s, 3H), 2.02-1.91 (m, 3H), 1.74-1.55 (m, 3H), 1.53-1.24 (m, 3H); LCMS (ESI, M+1): m/z=667.2.

Peak 2: Example 173 (41.1 mg, 31% yield) as white solid; ¹H NMR: (400 MHz, DMSO d6) δ 8.69 (d, J=7.6 Hz, 1H), 8.45 (s, 1H), 7.85 (d, J=3.6 Hz, 1H), 7.49-7.46 (m, 1H), 7.21 (s, 2H), 6.57-6.53 (m, 1H), 5.92 (s, 2H), 5.45-5.42 (m, 1H), 5.40-5.28 (m, 1H), 4.07-3.95 (m, 2H), 3.28-2.99 (m, 3H), 2.95-2.75 (m, 1H), 2.34 (s, 3H), 2.02-1.91 (m, 3H), 1.74-1.55 (m, 3H), 1.55-1.53 (m, 3H); LCMS (ESI, M+1): m/z=667.2.

### Example 174

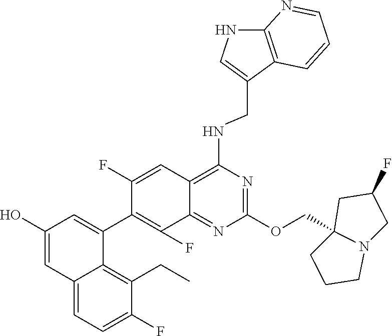 [SMILES: CCc1c(F)ccc2cc(O)cc(-c3c(F)cc4c(NCc5c[nH]c6ncccc56)nc(OC[C@@]56CCCN5C[C@H](F)C6)nc4c3F)c12]

### 4-(4-(((1H-pyrrolo[2,3-b]pyridin-3-yl)methyl)amino)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol

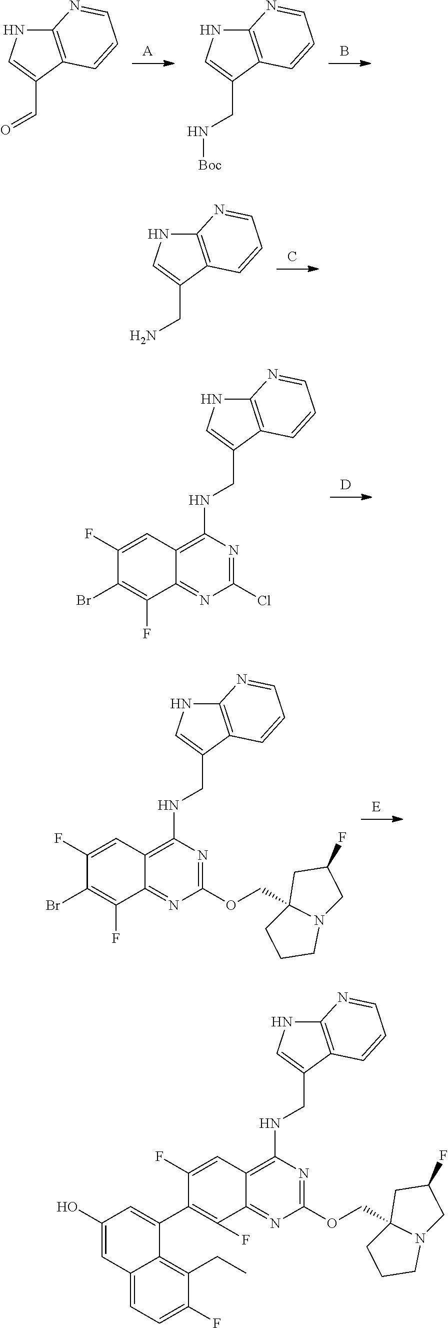

Step A. tert-butyl ((1H-pyrrolo[2,3-b]pyridin-3-yl)methyl)carbamate: To a solution of 1H-pyrrolo[2,3-b]pyridine-3-carbaldehyde (5 g, 1.0 equiv) in MeCN (50 mL) were added triethylsilane (11.9 g, 3 equiv), TFA (11.3 g, 2.9 equiv) and tert-butyl carbamate (4.01 g, 1 equiv). The reaction was stirred at 25° C. for 12 hours. tert-butyl carbamate (4.01 g, 1.0 equiv) and triethylsilane (3.64 g, 9.15e-1 equiv) were added. The reaction was stirred at 25° C. for 12 hours. The mixture was diluted with water (30 mL), neutralized with solid NaHCO₃ and extracted with EtOAc (2×30 mL). The combined organic layers were washed with brine (2×10 mL), dried over anhydrous sodium sulfate, concentrated and purified by reversed phase flash chromatography [C18, 0.1% formic acid condition] to afford the title compound (3.9 g, 45% yield) as white solid; LCMS (ESI, M+1): m/z=248.2.

Step B. (1H-pyrrolo[2,3-b]pyridin-3-yl)methanamine: To a solution of tert-butyl ((1H-pyrrolo[2,3-b]pyridin-3-yl)methyl)carbamate (3.9 g, 1.0 equiv) in MeOH (39 mL) was added HCl.MeOH (4 M, 39 mL) at 0° C. The reaction was stirred at 25° C. for 12 hours. The mixture was concentrated to afford the title compound (2.3 g, 95% yield) as white solid; LCMS (ESI, M+1): m/z=148.3.

Step C. N-((1H-pyrrolo[2,3-b]pyridin-3-yl)methyl)-7-bromo-2-chloro-6,8-difluoroquinazolin-4-amine: To a solution of 7-bromo-2,4-dichloro-6,8-difluoroquinazoline (1 g, 1.0 equiv) in MeCN (20 mL) was added DIEA (1.24 g, 3 equiv) and (1H-pyrrolo[2,3-b]pyridin-3-yl)methanamine (515 mg, 1.1 equiv) in MeCN (20 mL). The reaction was stirred at −40° C. for 3 hours. The mixture was diluted with water (10 mL) and extracted with EtOAc (2×10 mL). The combined organic layers were washed with brine (2×10 mL), dried over anhydrous sodium sulfate, concentrated to afford the title compound (1.2 g, 64% yield) as white solid; LCMS (ESI, M+1, M+3): m/z=424.1, 426.1.

Step D. N-((1H-pyrrolo[2,3-b]pyridin-3-yl)methyl)-7-bromo-6,8-difluoro-2-(((2R,7aS)-2-fluorotetrahydro-1H-pyrrolizin-7a(5H)-yl)methoxy)quinazolin-4-amine: To a solution of N-((1H-pyrrolo[2,3-b]pyridin-3-yl)methyl)-7-bromo-2-chloro-6,8-difluoroquinazolin-4-amine (200 mg, 0.33 equiv) in DMSO (0.02 mL) were added DIEA (548 mg, 3 equiv) and ((2R,7aS)-2-fluorotetrahydro-1H-pyrrolizin-7a(5H)-yl)methanol (2.25 g, 10 equiv). The reaction was stirred at 80° C. for 12 hours. The mixture was purified by reversed phase flash chromatography [C18, 0.1% formic acid condition] to afford the title compound (250 mg, 7% yield) as brown solid.

Step E. 4-(4-(((1H-pyrrolo[2,3-b]pyridin-3-yl)methyl)amino)-6,8-difluoro-2-(((2R,7aS)-2-fluorotetrahydro-1H-pyrrolizin-7a(5H)-yl)methoxy)quinazolin-7-yl)-5-ethyl-6-fluoronaphthalen-2-ol: To a solution of N-((1H-pyrrolo[2,3-b]pyridin-3-yl)methyl)-7-bromo-6,8-difluoro-2-(((2R,7aS)-2-fluorotetrahydro-1H-pyrrolizin-7a(5H)-yl)methoxy)quinazolin-4-amine (80 mg, 1.0 equiv), 5-ethyl-6-fluoro-4-(4,4,5,5-tetramethyl-1,3,2-dioxaborolan-2-yl)naphthalen-2-ol (69.3 mg, 1.5 equiv) in methoxycyclopentane (1 mL) were added K₃PO₄ (1.5 M, 292 μL, 3.0 equiv) and cataCxium® A Pd G3 (21.3 mg, 0.2 equiv). The mixture was degassed and purged with N₂ for 3 times. The reaction was stirred at 90° C. for 2 hours. The mixture was diluted with water (10 mL) and extracted with EtOAc (2×2 mL). The combined organic layers were dried over anhydrous sodium sulfate, concentrated and purified by reversed phase flash chromatography [C18, 0.1% formic acid condition] followed by prep-HPLC [Phenomenex luna C18 150×25 mm×10 μm; A: water(FA); B: CAN; B %: 15%-45% over 10 min] to afford the title compound (15.8 mg, 16% yield) as off-white solid; ¹H NMR (400 MHz, METHANOL-d₄) δ=8.26-8.17 (m, 2H), 7.86 (br d, J=9.2 Hz, 1H), 7.69 (dd, J=5.6, 8.8 Hz, 1H), 7.53 (s, 1H), 7.33-7.22 (m, 2H), 7.15 (dd, J=4.8, 7.6 Hz, 1H), 6.98 (d, J=2 Hz, 1H), 5.53-5.33 (m, 1H), 5.12-5.00 (m, 2H), 4.56-4.42 (m, 2H), 3.71-3.47 (m, 3H), 3.27-3.12 (m, 1H), 2.61-2.34 (m, 4H), 2.32-2.24 (m, 1H), 2.22-2.11 (m, 2H), 2.07-1.95 (m, 1H), 0.81 (t, J=7.6 Hz, 3H); LCMS (ESI, M+1): m/z=657.3.

### Example 175

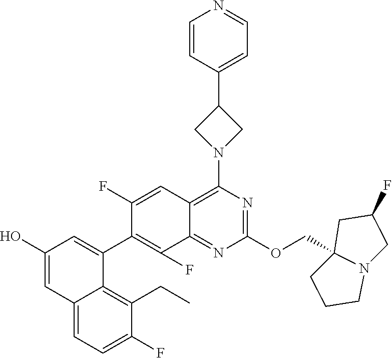 [SMILES: CCc1c(F)ccc2cc(O)cc(-c3c(F)cc4c(N5CC(c6ccncc6)C5)nc(OC[C@@]56CCCN5C[C@H](F)C6)nc4c3F)c12]

### 4-(6,8-difluoro-2-(((2R,7aS)-2-fluorotetrahydro-1H-pyrrolizin-7a(5H)-yl)methoxy)-4-(3-(pyridin-4-yl)azetidin-1-yl)quinazolin-7-yl)-5-ethyl-6-fluoronaphthalen-2-ol

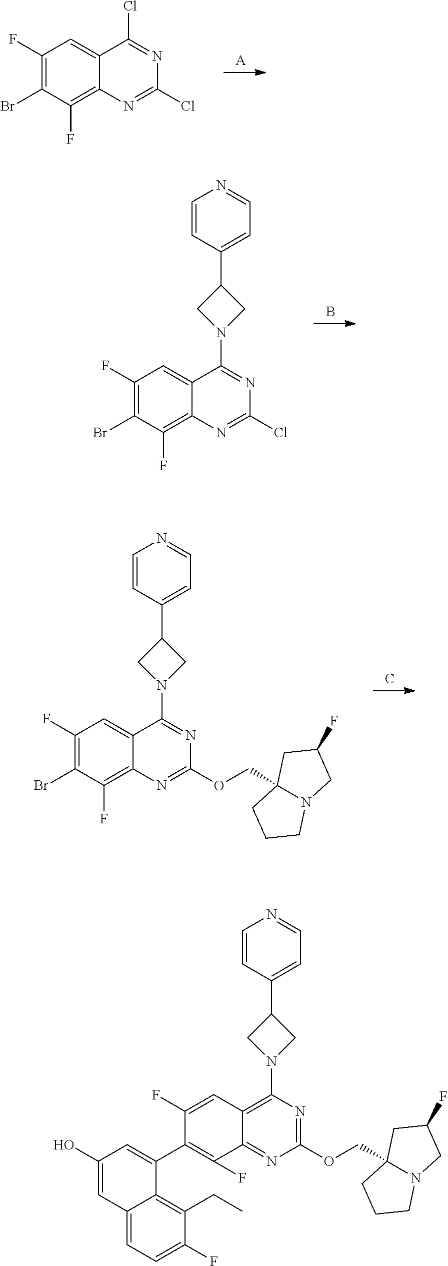

Step A. 7-bromo-2-chloro-6,8-difluoro-4-(3-(pyridin-4-yl)azetidin-1-yl)quinazoline: To a solution of 7-bromo-2,4-dichloro-6,8-difluoro-quinazoline (400 mg, 1.0 equiv) in DCM (20 mL) were added TEA (387 mg, 3.0 equiv) and 4-(azetidin-3-yl)pyridine dihydrochloride (250 mg, 1.0 equiv) at −40° C. The reaction was stirred at −40° C. for 1 hour. The mixture was diluted with water (10 mL) and extracted with dichloromethane (2×10 mL). The combined organic layers were dried over anhydrous sodium sulfate, concentrated to afford the title compound (800 mg, crude) as yellow solid; LCMS (ESI, M+1,M+3): m/z=411.0, 413.0.

Step B. 7-bromo-6,8-difluoro-2-(((2R,7aS)-2-fluorotetrahydro-1H-pyrrolizin-7a(5H)-yl)methoxy)-4-(3-(pyridin-4-yl)azetidin-1-yl)quinazoline: To a solution of 7-bromo-2-chloro-6,8-difluoro-4-(3-(pyridin-4-yl)azetidin-1-yl)quinazoline (550 mg, 1.0 equiv) and ((2R,7aS)-2-fluorotetrahydro-1H-pyrrolizin-7a(5H)-yl)methanol (2.13 g, 10 equiv) in DMSO (5 mL) was added TEA (2.91 g, 21 equiv). The reaction was stirred at 90° C. for 48 hours. The mixture was diluted with water (10 mL) and extracted with ethyl acetate (2×5 mL). The organic layers were dried over Na₂SO₄, concentrated and purified with reversed phase flash chromatography [C18, 0.1% NH₃.H₂O condition] followed by reversed phase flash chromatography [C18, 0.1% FA condition] to afford the title compound (60.0 mg, 8.4% yield) as white solid; LCMS (ESI, M+1, M+3): m/z=533.8, 535.8.

Step C. 4-(6,8-difluoro-2-(((2R,7aS)-2-fluorotetrahydro-1H-pyrrolizin-7a(5H)-yl)methoxy)-4-(3-(pyridin-4-yl)azetidin-1-yl)quinazolin-7-yl)-5-ethyl-6-fluoronaphthalen-2-ol: To a solution of 7-bromo-6,8-difluoro-2-(((2R,7aS)-2-fluorotetrahydro-1H-pyrrolizin-7a(5H)-yl)methoxy)-4-(3-(pyridin-4-yl)azetidin-1-yl)quinazoline (60 mg, 1.0 equiv), K₃PO₄ (1.5 M, 6.4 equiv) and 5-ethyl-6-fluoro-4-(4,4,5,5-tetramethyl-1,3,2-dioxaborolan-2-yl)naphthalen-2-ol (35.5 mg, 1.0 equiv) in methoxycyclopentane (2 mL) was added cataCXium-A-Pd-G3 (24.5 mg, 0.3 equiv). The reaction was stirred at 90° C. for 3 hours under N₂ atmosphere. The mixture was diluted with water (10 mL) and extracted with ethyl acetate (2×5 mL). The organic layers were dried over Na₂SO₄, concentrated and purified with reversed phase flash chromatography [C18, 0.1% FA condition] followed by prep-HPLC [Phenomenex luna C18 150×25 mm×10 μm; A: water (FA), B: ACN, B %: 18%-48% over 7 min] to afford the title compound (4.80 mg, 6.3% yield, HCOOH salt) as white solid; ¹H NMR (400 MHz, METHANOL-d4) δ=8.56 (br d, J=4.8 Hz, 2H), 7.73-7.59 (m, 2H), 7.57 (d, J=5.2 Hz, 2H), 7.29 (d, J=2.0 Hz, 1H), 7.25 (t, J=9.4 Hz, 1H), 6.97 (d, J=2.0 Hz, 1H), 5.45-5.24 (m, 1H), 5.15-5.01 (m, 2H), 4.78-4.63 (m, 2H), 4.43-4.17 (m, 3H), 3.42 (br s, 3H), 3.18-3.00 (m, 1H), 2.69-2.48 (m, 1H), 2.46-2.14 (m, 4H), 2.11-1.99 (m, 2H), 1.98-1.84 (m, 1H), 0.81 (br t, J=7.4 Hz, 3H); LCMS (ESI, M+1): m/z=644.3.

### Example 176

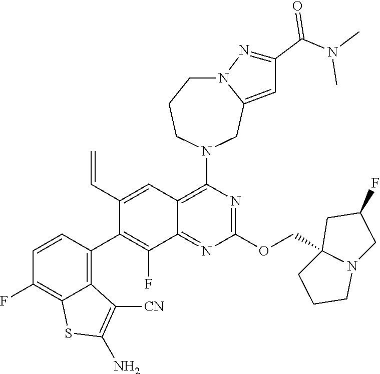 [SMILES: C=Cc1cc2c(N3CCCn4nc(C(=O)N(C)C)cc4C3)nc(OC[C@@]34CCCN3C[C@H](F)C4)nc2c(F)c1-c1ccc(F)c2sc(N)c(C#N)c12]

### 5-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide

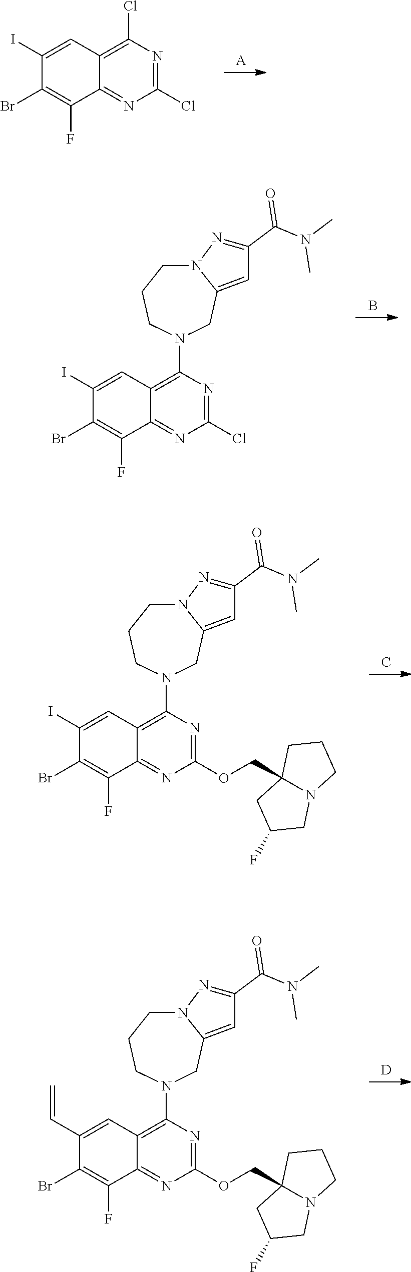

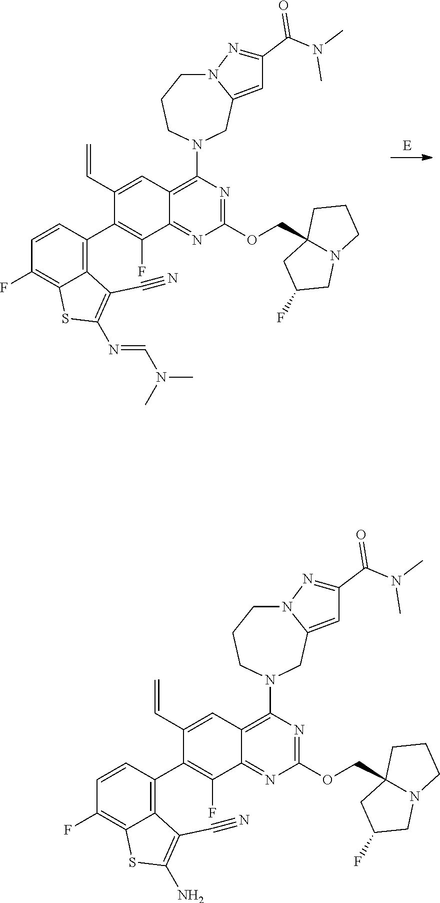

Step A. 5-(7-bromo-2-chloro-8-fluoro-6-iodoquinazolin-4-yl)-N,N-dimethyl-5,6,7,8-tetrahydro-4H-pyrazolo[1,5-a][1,4]diazepine-2-carboxamide: To a solution of 7-bromo-2,4-dichloro-8-fluoro-6-iodoquinazoline (1.0 g, 1.0 equiv) and DIEA (1.53 g, 5.0 equiv) in DCM (10 mL) was added N,N-dimethyl-5,6,7,8-tetrahydro-4H-pyrazolo[1,5-a][1,4]diazepine-2-carboxamide (444 mg, 0.90 equiv) at −40° C. The reaction was stirred at −40° C. for 1 hour. The mixture was diluted with water (40 mL) and extracted with DCM (3×30 mL). The combined organic layers were dried over anhydrous sodium sulfate, concentrated and purified with reversed phase flash chromatography [C18, 0.1% formic acid condition] to afford the title compound (1.00 g, 80% yield) as yellow solid; LCMS (ESI, M+1, M+3): m/z=592.9, 594.9.

Step B. 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide: A mixture of 5-(7-bromo-2-chloro-8-fluoro-6-iodoquinazolin-4-yl)-N,N-dimethyl-5,6,7,8-tetrahydro-4H-pyrazolo[1,5-a][1,4]diazepine-2-carboxamide (800 mg, 1.0 equiv) and ((2R,7aS)-2-fluorotetrahydro-1H-pyrrolizin-7a(5H)-yl)methanol (3.87 g, 20 equiv) was stirred at 100° C. for 12 hours. The reaction was diluted with water (30 mL) and extracted with EtOAc (3×20 mL). The combined organic layers were washed with brine (40 mL), dried over anhydrous sodium sulfate, concentrated and purified with reversed phase flash chromatography [C18, 0.1% formic acid condition] to afford the title compound (800 mg, 80% yield) as yellow solid; LCMS (ESI, M+1, M+3): m/z=716.2, 718.2.

Step C. 5-(7-bromo-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide: To a mixture of 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide (300 mg, 1.0 equiv), 4,4,5,5-tetramethyl-2-vinyl-1,3,2-dioxaborolane (64.5 mg, 1.0 equiv) and K₂CO₃ (1.5 M, 837 μL, 3.0 equiv) in dioxane (3 mL) was added Pd(dppf)Cl₂ (30.6 mg, 0.10 equiv). The reaction was degassed and purged with nitrogen 3 times. The reaction was stirred at 40° C. for 1 hour. The mixture was diluted with water (10 mL) and extracted with EtOAc (3×10 mL). The combined organic layers were washed with brine (30 mL), dried over anhydrous sodium sulfate, concentrated and purified with reversed phase flash chromatography [c18, 0.1% formic acid condition] to afford the title compound (160 mg, 58% yield) as yellow solid; LCMS (ESI, M+1, M+3): m/z=616.2, 618.2

Step D. 5-(7-(3-cyano-2-(((E)-(dimethylamino)methylene)amino)-7-fluorobenzo[b]thiophen-4-yl)-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide: To a mixture of 5-(7-bromo-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide (80.0 mg, 1.0 equiv), (E)-N′-(3-cyano-7-fluoro-4-(4,4,5,5-tetramethyl-1,3,2-dioxaborolan-2-yl)benzo[b]thiophen-2-yl)-N,N-dimethylformimidamide (72.7 mg, 1.5 equiv) and K₃P04 (1.5 M, 260 μL, 3.0 equiv) in THF (1 mL) was added APhos Pd G3 (8.24 mg, 0.10 equiv). The reaction was degassed and purged with nitrogen 3 times. The reaction was stirred at 80° C. for 3 hours. The mixture was diluted with water (5 mL) and extracted with EtOAc (3×5 mL). The combined organic layers were washed with brine (20 mL), dried over anhydrous sodium sulfate, concentrated and purified with prep-TLC [SiO₂, Dichloromethane: Methanol=10/1] to afford the title compound (50 mg, 48% yield) as yellow solid; LCMS (ESI, M+1): m/z=783.4.

Step E. 5-(7-(2-amino-3-cyano-7-fluorobenzo[b]thiophen-4-yl)-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide: To a solution of 5-(7-(3-cyano-2-(((E)-(dimethylamino)methylene)amino)-7-fluorobenzo[b]thiophen-4-yl)-8-fluoro-2-(((2R,7aS)-2-fluorotetrahydro-1H-pyrrolizin-7a(5H)-yl)methoxy)-6-vinylquinazolin-4-yl)-N,N-dimethyl-5,6,7,8-tetrahydro-4H-pyrazolo[1,5-a][1,4]diazepine-2-carboxamide (40.0 mg, 1.0 equiv) in DMAC (0.5 mL) was added K₃P04 (1.5 M, 0.5 mL). The reaction was stirred at 80° C. for 1 hour. The mixture was diluted with water (1 mL) and extracted with EtOAc (3×1 mL). The combined organic layers were washed with brine (5 mL), dried over anhydrous sodium sulfate, concentrated and purified with prep-HPLC [Phenomenex luna C18 150×25 mm×10 μm; A: water (FA), B: ACN; B %: 24%-54% over 10 min] and lyophilized to afford the title compound (13.5 mg, 35% yield, 0.16HCOOH) as off-white solid; ¹H NMR (400 MHz, METHANOL-d₄) δ=8.04 (s, 1H), 7.17-7.13 (m, 1H), 7.05-7.01 (m, 1H), 6.71 (s, 1H), 6.39-6.32 (m, 1H), 5.68 (d, J=17.2 Hz, 1H), 5.47-5.25 (m, 1H), 5.23-5.18 (m, 1H), 5.13 (s, 2H), 4.56-4.50 (m, 2H), 4.38-4.30 (m, 3H), 4.30-4.23 (m, 1H), 3.42-3.32 (m, 6H), 3.08 (s, 4H), 2.43-2.38 (m, 2H), 2.37-2.12 (m, 3H), 2.10-2.01 (m, 2H), 2.00-1.90 (m, 1H); LCMS (ESI, M+1): m/z=728.3.

### Example 177

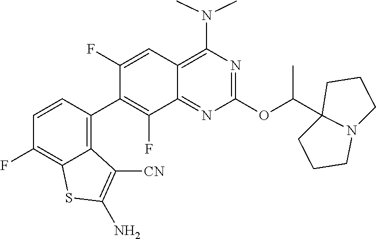 [SMILES: CC(Oc1nc(N(C)C)c2cc(F)c(-c3ccc(F)c4sc(N)c(C#N)c34)c(F)c2n1)C12CCCN1CCC2]

### 2-amino-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophene-3-carbonitrile (isomer 1)

### Example 178

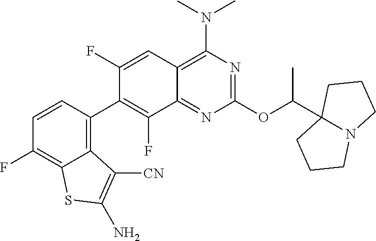 [SMILES: CC(Oc1nc(N(C)C)c2cc(F)c(-c3ccc(F)c4sc(N)c(C#N)c34)c(F)c2n1)C12CCCN1CCC2]

### 2-amino-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophene-3-carbonitrile (isomer 2)

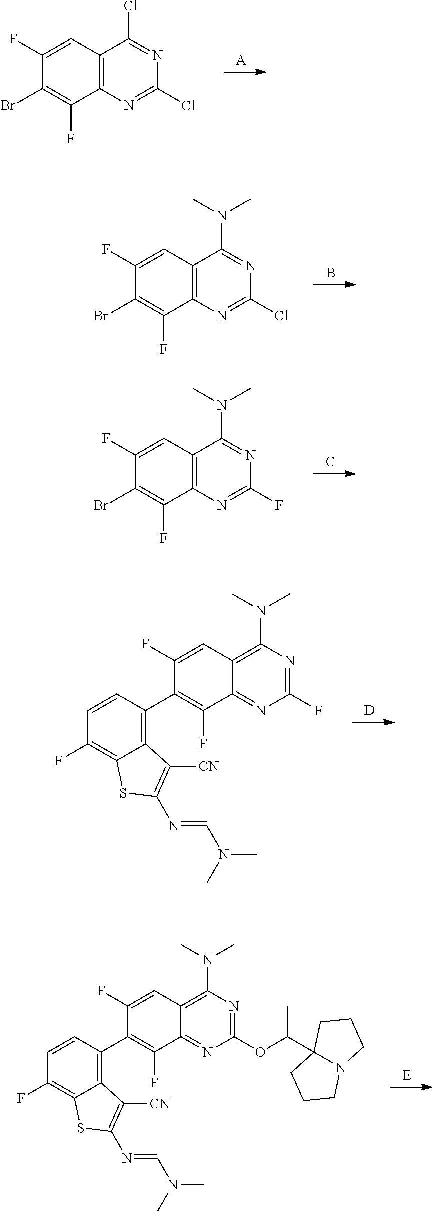

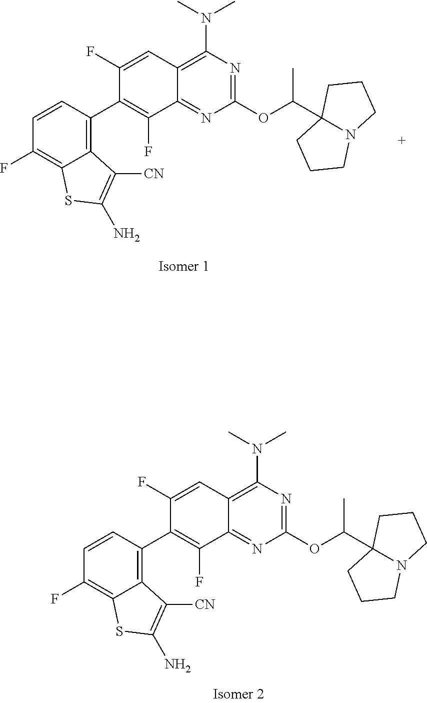 [SMILES: CC(Oc1nc(N(C)C)c2cc(F)c(-c3ccc(F)c4sc(N)c(C#N)c34)c(F)c2n1)C12CCCN1CCC2 ; CC(Oc1nc(N(C)C)c2cc(F)c(-c3ccc(F)c4sc(N)c(C#N)c34)c(F)c2n1)C12CCCN1CCC2]

Step A. 7-bromo-2-chloro-6,8-difluoro-N,N-dimethylquinazolin-4-amine: To a solution of 7-bromo-2,4-dichloro-6,8-difluoroquinazoline (1.00 g, 1.0 equiv) in DCM (10 mL) were added DIEA (1.65 g, 4.0 equiv) and N-methylmethanamine (260 mg, 1.0 equiv, HCl) at −40° C. The reaction was stirred at −40° C. for 1 hour. The mixture was diluted with water (20 mL) and extracted with DCM (20 mL×2). The combined organic layers were washed with brine (20 mL), dried over anhydrous sodium sulfate, concentrated and purified with column chromatography [SiO₂, petroleum ether/ethyl acetate=20/1 to 2/1] to afford the title compound (740 mg, 72% yield) as yellow solid; LCMS (ESI, M+1, M+3): m/z=321.9, 323.9.

Step B. 7-bromo-2,6,8-trifluoro-N,N-dimethylquinazolin-4-amine: To a solution of 7-bromo-2-chloro-6,8-difluoro-N,N-dimethylquinazolin-4-amine (740 mg, 1.0 equiv) in DMSO (8 mL) was added KF (2.67 g, 20 equiv). The reaction was stirred at 140° C. for 3 hours. The mixture was diluted with water (100 mL) and extracted with ethyl acetate (2×100 mL). The combined organic layers were washed with brine (100 mL), dried over anhydrous sodium sulfate, concentrated and triturated with MeOH (5 mL) at 25° C. for 10 minutes to afford the title compound (500 mg, 71% yield) as light yellow solid; LCMS (ESI, M+1, M+3): m/z=305.9, 307.9.

Step C. (E)-N′-3-cyano-4-(4-(dimethylamino)-2,6,8-trifluoroquinazolin-7-yl)-7-fluorobenzo[b]thiophen-2-yl)-N,N-dimethylformimidamide: To a solution of 7-bromo-2,6,8-trifluoro-N,N-dimethyl-quinazolin-4-amine (400 mg, 1.0 equiv) and N′-[3-cyano-7-fluoro-4-(4,4,5,5-tetramethyl-1,3,2-dioxaborolan-2-yl)benzothiophen-2-yl]-N,N-dimethyl-formamidine (488 mg, 1.0 equiv) in dioxane (10 mL) were added Cs₂CO₃ (1.28 g, 3.0 equiv) and PCy₃ Pd G3 (85.0 mg, 0.1 equiv). The reaction was degassed and purged with N₂ for 3 times and stirred at 90° C. for 2 hours under N₂ atmosphere. The mixture was diluted with water (50 mL) and extracted with ethyl acetate (50 mL×2). The combined organic layers were washed with brine (50 mL), dried over anhydrous sodium sulfate, concentrated and purified with prep-TLC [SiO₂, petroleum ether/ethyl acetate=1/1] to afford the title compound (85.0 mg, 14% yield) as yellow solid; LCMS (ESI, M+1): m/z=473.1.

Step D. (E)-N′-(3-cyano-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophen-2-yl)-N,N-dimethylformimidamide: To a solution of 1-(tetrahydro-1H-pyrrolizin-7a(5H)-yl)ethan-1-ol (34.5 mg, 1.5 equiv) in THE (3 mL) was added NaH (8.89 mg, 1.5 equiv, 60% purity) at 0° C. The reaction was stirred at 0° C. for 0.5 hours. (E)-N′-(3-cyano-4-(4-(dimethylamino)-2,6,8-trifluoroquinazolin-7-yl)-7-fluorobenzo[b]thiophen-2-yl)-N,N-dimethylformimidamide (70.0 mg, 1.0 equiv) in THE (1 mL) was added and the reaction was stirred at 25° C. for 1 hour. The mixture was quenched with water (10 mL) at 0° C. and extracted with ethyl acetate (10 mL×2). The combined organic layers were washed with brine (10 mL), dried over anhydrous sodium sulfate, concentrated and purified with prep-HPLC [FA condition; column: Waters Xbridge 150×25 mm×5 μm; mobile phase: water(FA)−ACN; gradient: 18%-48% B over 9 min] to afford the title compound (20.0 mg, 22% yield) as yellow solid; LCMS (ESI, M+1): m/z=608.2.

Step E. 2-amino-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophene-3-carbonitrile: To a solution of (E)-N′-(3-cyano-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophen-2-yl)-N,N-dimethylformimidamide (20 mg, 1.0 equiv) in DMAc (2 mL) was added K₃P04 (2.00 mL, 2 M). The reaction was stirred at 80° C. for 1 hour. The mixture was diluted with water (10 mL) and extracted with ethyl acetate (10 mL×2). The combined organic layers were washed with brine (10 mL), dried over anhydrous sodium sulfate, concentrated and purified with prep-HPLC [TFA condition; column: Phenomenex luna C18 150×25 mm×10 μm; mobile phase: water (TFA)−ACN; gradient: 20%-50% B over 14 min] to afford two isomers.

Peak 1, Example 177: 2-amino-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophene-3-carbonitrile (6.53 mg, 27% yield, TFA) as yellow solid; ¹H NMR (400 MHz, METHANOL-d₄) δ=7.93 (br d, J=10.4 Hz, 1H), 7.31 (br dd, J=5.2, 8.0 Hz, 1H), 7.06 (t, J=8.8 Hz, 1H), 5.38 (br d, J=6.0 Hz, 1H), 3.73-3.67 (m, 2H), 3.56 (d, J=8.0 Hz, 6H), 3.29-3.27 (m, 1H), 2.68-2.49 (m, 1H), 2.28-2.07 (m, 8H), 1.58 (br d, J=6.0 Hz, 3H); LCMS (ESI, M+1): m/z=553.2.

Peak 2, Example 178: 2-amino-4-(4-(dimethylamino)-6,8-difluoro-2-(1-(tetrahydro-1H-pyrrolizin-7a(5H)-yl)ethoxy)quinazolin-7-yl)-7-fluorobenzo[b]thiophene-3-carbonitrile (5.29 mg, 21% yield, 1CF₃COOH) as yellow solid; ¹H NMR (400 MHz, METHANOL-d₄) δ=7.96 (br d, J=10.0 Hz, 1H), 7.32 (br dd, J=5.2, 8.4 Hz, 1H), 7.07 (t, J=8.8 Hz, 1H), 5.44 (br d, J=5.2 Hz, 1H), 3.76-3.63 (m, 2H), 3.60-3.53 (m, 6H), 3.27-3.21 (m, 1H), 2.67-2.53 (m, 1H), 2.32-1.94 (m, 8H), 1.58 (br d, J=5.6 Hz, 3H); LCMS (ESI, M+1): m/z=553.2.

### Example 179

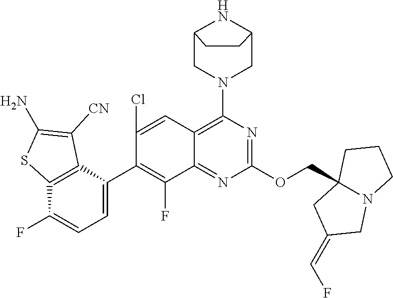

### (S)-4-(4-((1R,5S)-3,8-diazabicyclo[3.2.1]octan-3-yl)-6-chloro-8-fluoro-2-(((S,Z)-2-(fluoromethylene)tetrahydro-1H-pyrrolizin-7a(5H)-yl)methoxy)quinazolin-7-yl)-2-amino-7-fluorobenzo[b]thiophene-3-carbonitrile

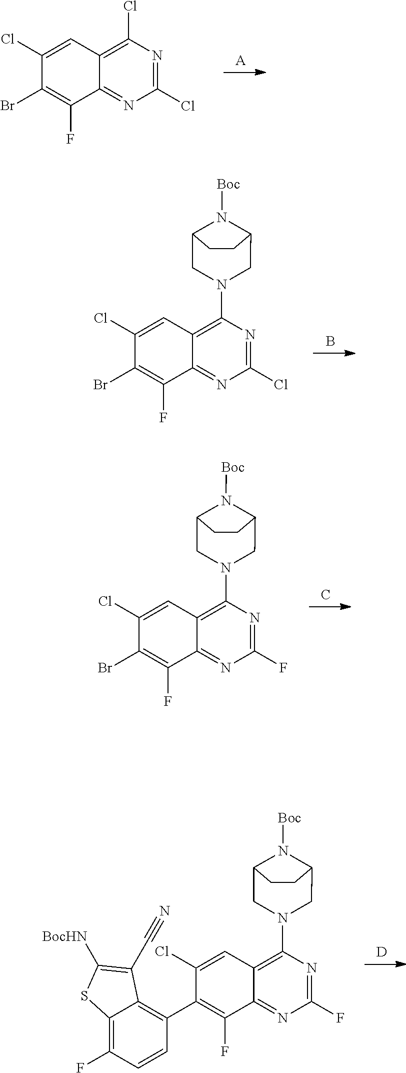

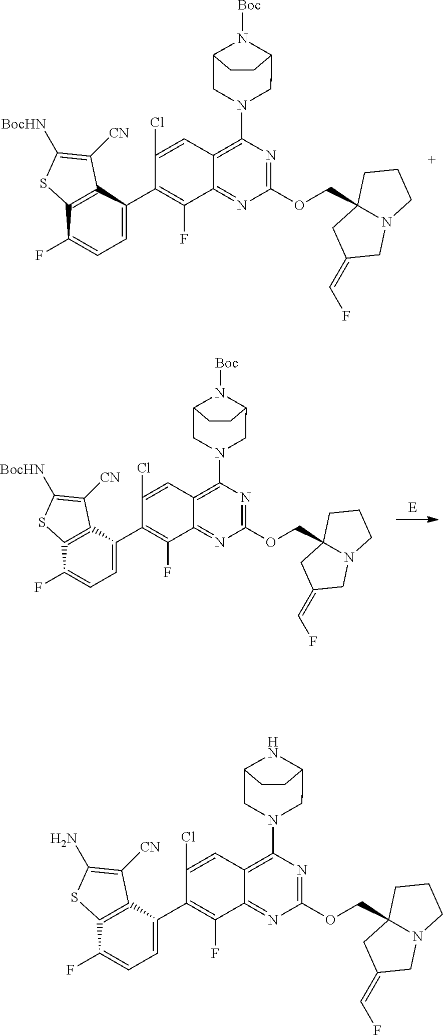

Step A. tert-butyl (1R,5S)-3-(7-bromo-2,6-dichloro-8-fluoroquinazolin-4-yl)-3,8-diazabicyclo[3.2.1]octane-8-carboxylate: To a solution of 7-bromo-2,4,6-trichloro-8-fluoro-quinazoline (5 g, 1.0 equiv) and TEA (4.59 g, 3.0 equiv) in DCM (200 mL) was added tert-butyl (1R,5S)-3,8-diazabicyclo[3.2.1]octane-8-carboxylate (3.21 g, 1.0 equiv) at −40° C. The reaction was stirred at −40° C. for 3 hours. The mixture was quenched with water (50 mL) and extracted with dichloromethane (2×50 mL). The combined organic layers were dried over anhydrous sodium sulfate, concentrated and purified with silica gel chromatography (SiO₂, Petroleum ether/Ethyl acetate=30/1 to 20/1) to afford the title compound (6.14 g, 75% yield) as yellow solid; LCMS (ESI, M+1, M+3): m/z=505.1, 507.2.

Step B. tert-butyl (1R,5S)-3-(7-bromo-6-chloro-2,8-difluoroquinazolin-4-yl)-3,8-diazabicyclo[3.2.1]octane-8-carboxylate: To a mixture of tert-butyl (1R,5S)-3-(7-bromo-2,6-dichloro-8-fluoroquinazolin-4-yl)-3,8-diazabicyclo[3.2.1]octane-8-carboxylate (4.14 g, 1.0 equiv) and KF (4.75 g, 10 equiv) in DMSO (50 mL) was added 18-CROWN-6 (216 mg, 0.1 equiv). The reaction was stirred at 120° C. for 4 hours. The mixture was diluted with water (100 mL) and extracted with ethyl acetate (2×50 mL). The combined organic layers were dried over anhydrous sodium sulfate, concentrated to afford the title compound (4 g, crude) as yellow solid; LCMS (ESI, M+1,M+3): m/z=489.1, 491.1.

Step C. tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate: A mixture of tert-butyl (1R,5S)-3-(7-bromo-6-chloro-2,8-difluoroquinazolin-4-yl)-3,8-diazabicyclo[3.2.1]octane-8-carboxylate (4 g, 1.0 equiv), tert-butyl (3-cyano-4-(5,5-dimethyl-1,3,2-dioxaborinan-2-yl)-7-fluorobenzo[b]thiophen-2-yl)carbamate (4.95 g, 1.5 equiv), Cs₂CO₃ (7.98 g, 3.0 equiv) and DPEphosPdCl₂ (1.12 g, 0.2 equiv) in THE (50.0 mL) was degassed and purged with N₂ for 3 times, and then the mixture was stirred at 60° C. for 1 hour under N₂ atmosphere. The mixture was diluted with H₂O (100 mL), extracted with ethyl acetate (2×50 mL). The combined organic layers were dried over anhydrous Na₂SO₄, concentrated under vacuum and purified with reversed phase flash [C18, 0.1% formic acid condition] to afford the title compound (1.83 g, 31% yield) as yellow solid; LCMS (ESI, M+1): m/z=701.3.

Step D. tert-butyl (1R,5S)-3-(7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate: To a solution of (S,Z)-(2-(fluoromethylene)tetrahydro-1H-pyrrolizin-7a(5H)-yl)methanol (215 mg, 1.1 equiv) in THE (10.0 mL) was added NaH (137 mg, 60% purity, 3.0 equiv) at 0° C. The reaction was stirred at 0° C. for 0.5 hours, then tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (800 mg, 1.0 equiv) was added. The reaction was stirred at 25° C. for 1 hour. The mixture was quenched with water (50 mL) and extracted with ethyl acetate (2×25 mL). The combined organic layers were dried over anhydrous sodium sulfate, concentrated and purified with SFC separation [column: DAICEL CHIRALPAK AD, 250 mm×30 mm, 10 μm; A: CO₂, B: PrOH(0.1% NH₃H₂O), B %: 25% B over 3.8 min] to afford two isomers.

tert-butyl (1R,5S)-3-(7-((S)-2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate (385 mg, 38% yield) as yellow solid; LCMS (ESI, M+1): m/z=852.3; SFC>99% ee, column: Chiralcel AD-350×4.6 mm I.D., 3 μm; mobile phase: 5% to 40% IPA (0.05% DEA) in CO₂, flow rate: 3 mL/min, detector: PDA, t_{R}: 1.606 min.

tert-butyl (1R,5S)-3-(7-((R)-2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate (500 mg, 51% yield) as yellow solid; LCMS (ESI, M+1): m/z=852.3; SFC>97.6% ee, column: Chiralcel AD-350×4.6 mm I.D., 3 μm; mobile phase: 5% to 40% IPA (0.05% DEA) in CO₂, flow rate: 3 mL/min, detector: PDA, t_{R}: 1.848 min.

Step E. (S)-4-(4-((1R,5S)-3,8-diazabicyclo[3.2.1]octan-3-yl)-6-chloro-8-fluoro-2-(((S,Z)-2-(fluoromethylene)tetrahydro-1H-pyrrolizin-7a(5H)-yl)methoxy)quinazolin-7-yl)-2-amino-7-fluorobenzo[b]thiophene-3-carbonitrile: To a solution of tert-butyl (1R,5S)-3-(7-((S)-2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-3,8-diazabicyclo[3.2.1]octane-8-carboxylate (380 mg, 1.0 equiv) in DCM (4 mL) was added TFA (4 mL). The reaction was stirred at 20° C. for 1 hour. The mixture was concentrated under vacuum. The mixture was basified by saturated NaHCO₃ solution (10 mL) and extracted with ethyl acetate (2×10 mL). The organic layers were dried over Na₂SO₄, concentrated and purified with prep-HPLC[Waters Xbridge 150×25 mm×5 μm; A: water (water(NH₄HCO₃)−ACN); B: ACN; B %: 48%-78% over 9 min] to afford the title compound (65.7 mg, 22% yield) as off-white solid; ¹H NMR (400 MHz, METHANOL-d4) δ=7.86 (d, J=1.2 Hz, 1H), 7.20 (dd, J=5.2, 8.4 Hz, 1H), 7.03 (t, J=8.8 Hz, 1H), 6.80-6.47 (m, 1H), 4.56-4.36 (m, 2H), 4.32-4.16 (m, 2H), 3.84 (br d, J=14.8 Hz, 1H), 3.69-3.54 (m, 4H), 3.45 (br d, J=14.4 Hz, 1H), 3.21-3.07 (m, 1H), 2.80-2.61 (m, 2H), 2.43 (br d, J=16.0 Hz, 1H), 2.22-2.06 (m, 1H), 2.04-1.84 (m, 3H), 1.82 (br s, 4H); LCMS (ESI,M+1): m/z=652.3.

### Example 180

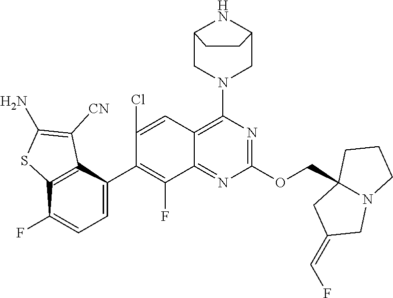

### (R)-4-(4-((1R,5S)-3,8-diazabicyclo[3.2.1]octan-3-yl)-6-chloro-8-fluoro-2-(((S,Z)-2-(fluoromethylene)tetrahydro-1H-pyrrolizin-7a(5H)-yl)methoxy)quinazolin-7-yl)-2-amino-7-fluorobenzo[b]thiophene-3-carbonitrile

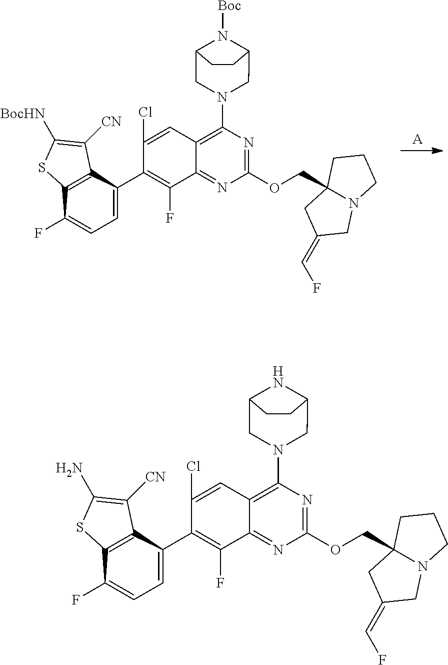

Step A. 4-((R)-4-((1R,5S)-3,8-diazabicyclo[3.2.1]octan-3-yl)-6-chloro-8-fluoro-2-(((S,Z)-2-(fluoromethylene)tetrahydro-1H-pyrrolizin-7a(5H)-yl)methoxy)quinazolin-7-yl)-2-amino-7-fluorobenzo[b]thiophene-3-carbonitrile: To a solution of tert-butyl (1R,5S)-3-((R)-7-(2-((tert-butoxycarbonyl)amino)-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)guinazolin-4-yl)-3,8-diazabicyclo[3.2.1]octane-8-carboxylate(10.4 g, 1.0 equiv) in DCM (100 mL) was added TFA (100 mL). The reaction was stirred at 0-20° C. for 1 hour. The mixture was concentrated under vacuum. The mixture was basified by saturated NaHCO₃ solution (200 mL) and extracted with ethyl acetate (2×100 mL). The combined organic layers were washed with brine (80 mL), dried over anhydrous sodium sulfate, concentrated and purified with prep-HPLC [SiO₂ column: Welch Ultimate XB-SiOH 250×70×10 μm; A: Hexane, B:EtOH (0.1% NH₃.H₂O) B %:20%-60% over 15 min] to afford the title compound (5.27 g, 66% yield) as yellow solid; ¹H NMR (400 MHz, METHANOL-d4) δ=7.85 (d, J=1.6 Hz, 1H), 7.20 (dd, J=5.2, 8.4 Hz, 1H), 7.03 (dd, J=8.4, 9.4 Hz, 1H), 6.76-6.51 (m, 1H), 4.56-4.39 (m, 2H), 4.32-4.19 (m, 2H), 3.85 (br d, J=14.8 Hz, 1H), 3.68-3.54 (m, 4H), 3.45 (br d, J=14.4 Hz, 1H), 3.20-3.10 (m, 1H), 2.79-2.66 (m, 2H), 2.43 (br d, J=15.6 Hz, 1H), 2.19-2.08 (m, 1H), 2.04-1.89 (m, 2H), 1.88-1.75 (m, 5H); LCMS (ESI,M+1): m/z=652.2.

### Example 181

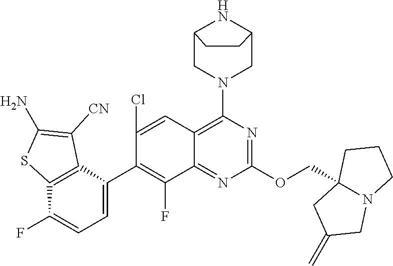

### (S)-4-(4-((1R,5S)-3,8-diazabicyclo[3.2.1]octan-3-yl)-6-chloro-8-fluoro-2-(((R)-2-methylenetetrahydro-1H-pyrrolizin-7a(5H)-yl)methoxy)quinazolin-7-yl)-2-amino-7-fluorobenzo[b]thiophene-3-carbonitrile

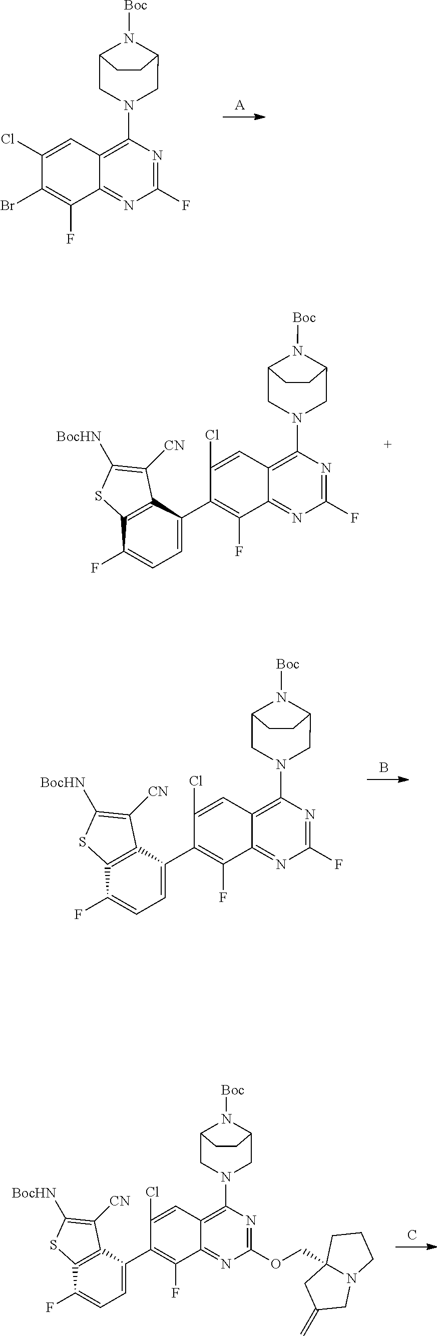

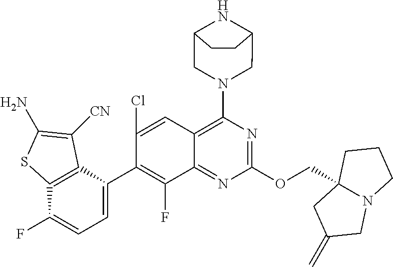

Step A. tert-butyl (1R,5S)-3-(7-(2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate: To a mixture of tert-butyl (1R,5S)-3-(7-bromo-6-chloro-2,8-difluoroquinazolin-4-yl)-3,8-diazabicyclo[3.2.1]octane-8-carboxylate (100 g, 1.0 equiv) and tert-butyl (3-cyano-4-(5,5-dimethyl-1,3,2-dioxaborinan-2-yl)-7-fluorobenzo[b]thiophen-2-yl)carbamate (99.1 g, 1.2 equiv) in THE (2000 mL) were added Cs₂CO₃ (200 g, 3.0 equiv) and DPEphosPdCl₂ (14 g, 0.1 equiv). The reaction was degassed and purged with nitrogen 3 times. The reaction was stirred at 60° C. for 5 hours. The mixture was diluted with H₂O (1000 mL) and extracted with ethyl acetate (2×500 mL). The combined organic layers were dried over anhydrous sodium sulfate, concentrated and purified with column chromatography (SiO₂, petroleum ether/ethyl acetate 15/1 to 7/1) followed by reversed phase flash [C18, 0.1% formic acid condition]. Then the residue was purified with SFC separation [column: DAICEL CHIRALCEL OX, 250 mm×50 mm, 10 μm; A: CO₂, B: EtOH(0.1% NH₃H₂O), B %: 40% B over 5.2 min] to afford two isomers.

tert-butyl (1R,5S)-3-(7-((S)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (42.5 g, 45% yield) as yellow solid; LCMS (ESI, M+1): m/z=701.1; SFC>99% ee, column: Chiralcel OX-3 50×4.6 mm I.D., 3 μm; mobile phase: 5% to 40% EtOH(0.05% DEA) in CO₂, flow rate: 3 mL/min, detector: 220 nm, t_{R}: 1.725 min.

Tert-butyl (1R,5S)-3-(7-((R)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (43.7 g, 48% yield) as yellow solid; LCMS (ESI, M+1): m/z=701.2; SFC>99% ee, column: Chiralcel OX-3 50×4.6 mm I.D., 3 μm; mobile phase: 5% to 40% EtOH(0.05% DEA) in CO₂, flow rate: 3 mL/min, detector: 220 nm, t_{R}: 2.029 min.

Step B. tert-butyl (1R,5S)-3-(7-((S)-2-((tert-butoxycarbonyl)amino)-3-cyano-7-fluorobenzo[b]thiophen-4-yl)-6-chloro-8-fluoro-2-(((R)-2-methylenetetrahydro-1H-pyrrolizin-7a(5H)-yl)methoxy)quinazolin-4-yl)-3,8-diazabicyclo[3.2.1]octane-8-carboxylate: To a solution of (R)-(2-methylenetetrahydro-1H-pyrrolizin-7a(5H)-yl)methanol (52.4 mg, 1.2 equiv) in THE (4 mL) was added NaH (34.2 mg, 60% purity, 3.0 equiv) at 0° C. The reaction was stirred at 20° C. for 0.5 hours. tert-butyl (1R,5S)-3-(7-((S)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (200 mg, 1.0 equiv) was added and the reaction was stirred at 20° C. for 0.5 hours. The mixture was quenched with water (4 mL) and layers were separated. The aqueous phase was extracted with ethyl acetate (3×3.0 mL). The combined organic layers were dried over anhydrous Na₂SO₄, concentrated and purified by reversed phase flash [water (0.1% FA)/acetonitrile] to afford the title compound (152 mg, 60.5% yield) as a yellow solid; LCMS (ESI, M+1): m/z=834.3.

Step C. (S)-4-(4-((1R,5S)-3,8-diazabicyclo[3.2.1]octan-3-yl)-6-chloro-8-fluoro-2-(((R)-2-methylenetetrahydro-1H-pyrrolizin-7a(5H)-yl)methoxy)quinazolin-7-yl)-2-amino-7-fluorobenzo[b]thiophene-3-carbonitrile: To a solution of tert-butyl (1R,5S)-3-(7-((S)-2-((tert-butoxycarbonyl)amino)-3-cyano-7-fluorobenzo[b]thiophen-4-yl)-6-chloro-8-fluoro-2-(((R)-2-methylenetetrahydro-1H-pyrrolizin-7a(5H)-yl)methoxy)quinazolin-4-yl)-3,8-diazabicyclo[3.2.1]octane-8-carboxylate (170 mg, 1.0 equiv) in DCM (2 mL) was added TFA (3.07 g, 132 equiv). The reaction was stirred at 20° C. for 0.5 hours. The mixture was concentrated in vacuum and basified with saturated aqueous NaHCO₃(5 mL). The aqueous phase was extracted with ethyl acetate (3×4 mL). The combined organic layers were dried over Na₂SO₄, concentrated and purified by prep-HPLC [column: Phenomenex luna C18 250×50 mm×15 μm; A: water (FA), B: ACN; B %: 12%-42% over 9 min] to afford the title compound (62.1 mg, 46% yield) as a white solid; ¹H NMR (400 MHz, methanol-d₄) δ=7.85 (d, J=1.6 Hz, 1H), 7.20 (dd, J=5.2, 8.4 Hz, 1H), 7.03 (dd, J=8.4, 9.6 Hz, 1H), 5.00-4.94 (m, 2H), 4.56-4.38 (m, 2H), 4.27-4.17 (m, 2H), 3.73-3.54 (m, 5H), 3.30-3.26 (m, 1H), 3.16-3.08 (m, 1H), 2.82-2.74 (m, 1H), 2.73-2.65 (m, 1H), 2.50-2.42 (m, 1H), 2.19-2.09 (m, 1H), 2.03-1.87 (m, 2H), 1.87-1.76 (m, 5H); LCMS (ESI, M+1): m/z=634.3.

### Example 182

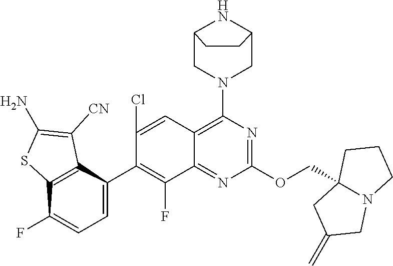

### (R)-4-(4-((1R,5S)-3,8-diazabicyclo[3.2.1]octan-3-yl)-6-chloro-8-fluoro-2-(((R)-2-methylenetetrahydro-1H-pyrrolizin-7a(5H)-yl)methoxy)quinazolin-7-yl)-2-amino-7-fluorobenzo[b]thiophene-3-carbonitrile

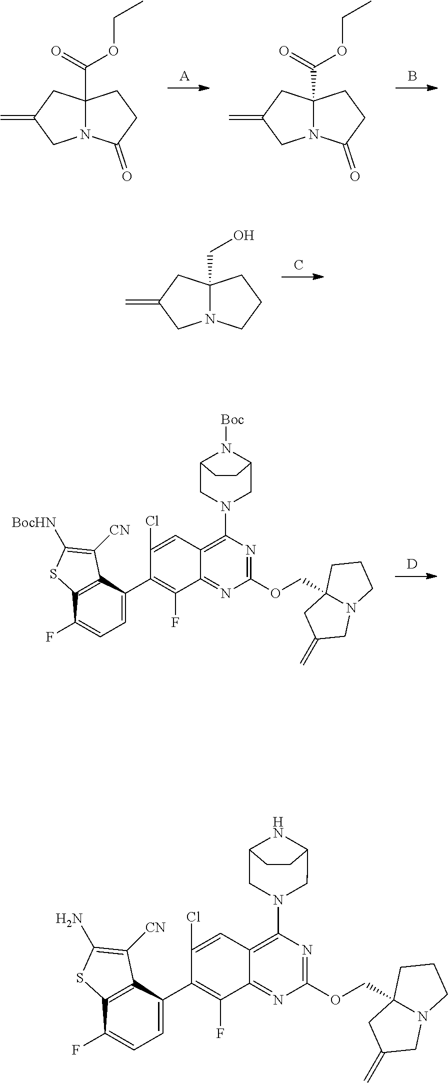

Step A. ethyl (R)-2-methylene-5-oxotetrahydro-1H-pyrrolizine-7a(5H)-carboxylate: Ethyl 2-methylene-5-oxotetrahydro-1H-pyrrolizine-7a(5H)-carboxylate (10 g) was separated by SFC [column: DAICEL CHIRALPAK IK(250 mm×50 mm, 10 μm); mobile phase: CO₂-i-PrOH(0.1% NH₃.H₂O); B %:0%, isocratic elution mode] to afford the title compound (4.40 g, 42% yield) as light yellow gum; LCMS (ESI, M+1): m/z=210.1.

Step B. (R)-(2-methylenetetrahydro-1H-pyrrolizin-7a(5H)-yl)methanol: To a solution of ethyl (R)-2-methylene-5-oxotetrahydro-1H-pyrrolizine-7a(5H)-carboxylate (1.00 g, 1.0 equiv) in THF (10.0 mL) was slowly added DIBAL-H (1 M, 10 equiv) at 0° C. The reaction was stirred at 0° C. for 5 hours. The mixture was quenched with water (2.4 mL), 15% NaOH (2.4 mL) was added into the mixture and water (7.2 mL) was added. The mixture was dried over anhydrous sodium sulfate, concentrated under vacuum. The mixture was purified with column chromatography (SiO₂, petroleum ether/ethyl acetate 100/1 to ethyl acetate: Methanol=10/1) to afford the title compound (170 mg, 19% yield) as yellow oil; ¹H NMR (400 MHz, CHLOROFORM-d) δ=4.94-4.87 (m, 2H), 3.64 (br d, J=14.6 Hz, 1H), 3.36-3.21 (m, 3H), 3.15-3.02 (m, 2H), 2.70-2.61 (m, 1H), 2.50-2.41 (m, 1H), 2.36-2.26 (m, 1H), 1.98-1.75 (m, 3H), 1.73-1.62 (m, 1H); LCMS (ESI, M+1): m/z=154.2.

Step C. tert-butyl 3-(7-((R)-2-((tert-butoxycarbonyl)amino)-3-cyano-7-fluorobenzo[b]thiophen-4-yl)-6-chloro-8-fluoro-2-(((R)-2-methylenetetrahydro-1H-pyrrolizin-7a(5H)-yl)methoxy)quinazolin-4-yl)-3,8-diazabicyclo[3.2.1]octane-8-carboxylate: To a solution of (R)-(2-methylenetetrahydro-1H-pyrrolizin-7a(5H)-yl)methanol (52.5 mg, 1.2 equiv) in THE (2.00 mL) was added NaH (34.2 mg, 60% purity, 3.0 equiv) at 0° C. The reaction was stirred at 0° C. for 0.5 hours. tert-butyl (1R,5S)-3-(7-((R)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (200 mg, 1.0 equiv) was added into the mixture and the reaction was stirred at 10° C. for 0.5 hours. The mixture was quenched with water(20 mL) and extracted with ethyl acetate (2×10 mL). The combined organic layers were dried over anhydrous sodium sulfate, concentrated under vacuum to afford the title compound (300 mg, crude) as yellow solid; LCMS (ESI, M+1): m/z=834.3.

Step D. tert-butyl 3-(7-((R)-2-((tert-butoxycarbonyl)amino)-3-cyano-7-fluorobenzo[b]thiophen-4-yl)-6-chloro-8-fluoro-2-(((R)-2-methylenetetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-((R)-2-((tert-butoxycarbonyl)amino)-3-cyano-7-fluorobenzo[b]thiophen-4-yl)-6-chloro-8-fluoro-2-(((R)-2-methylenetetrahydro-1H-pyrrolizin-7a(5H)-yl)methoxy)quinazolin-4-yl)-3,8-diazabicyclo[3.2.1]octane-8-carboxylate (290 mg, 1.0 equiv) in DCM (5.00 mL) was added TFA (5 mL). The reaction was stirred at 20° C. for 1 hour. The mixture was concentrated under vacuum and the mixture was basified by saturated NaHCO₃ solution (50 mL) and extracted with ethyl acetate (2×25 mL) The combined organic layers were washed with brine (30 mL), dried over anhydrous sodium sulfate, concentrated and purified by prep-HPLC [column: Phenomenex luna C18 150×25 mm×10 μm; A: water(FA), B: ACN; B %:12%-42% over 9 min] followed by prep-HPLC [column: Waters Xbridge 150×25 mm×5 μm; B: water(NH₄HCO₃), B: ACN; B %:30%-60% over 9 min] to afford the title compound (13.0 g, 66% yield) as yellow solid. ¹H NMR (400 MHz, METHANOL-d4) δ=7.85 (d, J=1.6 Hz, 1H), 7.20 (dd, J=5.2, 8.4 Hz, 1H), 7.03 (t, J=8.8 Hz, 1H), 5.02-4.94 (m, 2H), 4.56-4.37 (m, 2H), 4.27-4.17 (m, 2H), 3.76-3.53 (m, 5H), 3.29 (br s, 1H), 3.13 (td, J=5.2, 10.8 Hz, 1H), 2.78 (br d, J=16.0 Hz, 1H), 2.74-2.65 (m, 1H), 2.46 (br d, J=16.0 Hz, 1H), 2.18-2.10 (m, 1H), 2.02-1.93 (m, 1H), 1.92-1.72 (m, 6H); LCMS (ESI, M+1): m/z=634.3.

### Example 183

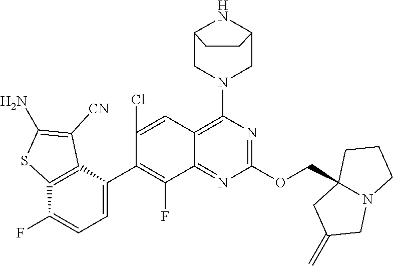

### (S)-4-(4-((1R,5S)-3,8-diazabicyclo[3.2.1]octan-3-yl)-6-chloro-8-fluoro-2-(((S)-2-methylenetetrahydro-1H-pyrrolizin-7a(5H)-yl)methoxy)quinazolin-7-yl)-2-amino-7-fluorobenzo[b]thiophene-3-carbonitrile

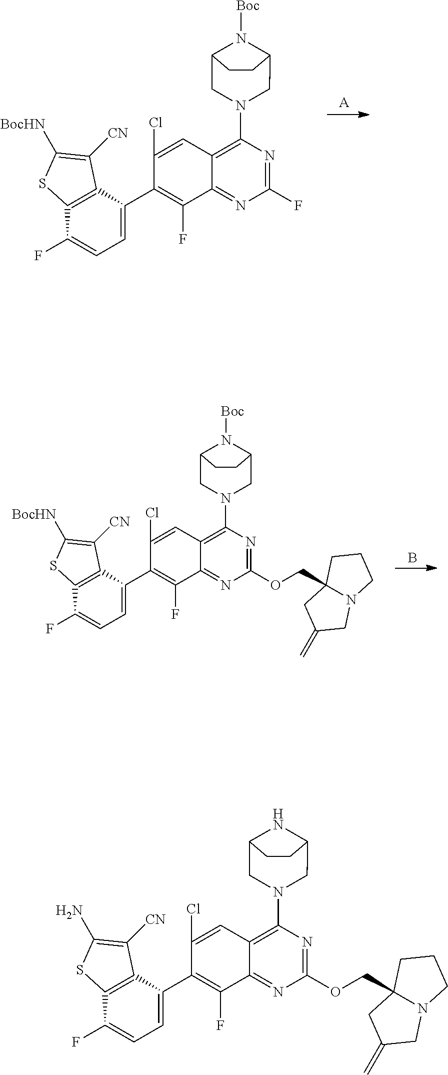

Step A. tert-butyl (1R,5S)-3-(7-((S)-2-((tert-butoxycarbonyl)amino)-3-cyano-7-fluorobenzo[b]thiophen-4-yl)-6-chloro-8-fluoro-2-(((S)-2-methylenetetrahydro-1H-pyrrolizin-7a(5H)-yl)methoxy)quinazolin-4-yl)-3,8-diazabicyclo[3.2.1]octane-8-carboxylate:

To a solution of (R)-(2-methylenetetrahydro-1H-pyrrolizin-7a(5H)-yl)methanol (52.4 mg, 1.2 equiv) in THE (4 mL) was added NaH (34.2 mg, 60% purity, 3.0 equiv) at 0° C. The reaction was stirred at 20° C. for 0.5 hours. Tert-butyl (1R,5S)-3-(7-((S)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (200 mg, 1.0 equiv) was added and the reaction was stirred at 20° C. for 0.5 hours. The mixture was quenched with water (5 mL) and layers were separated. The aqueous phase was extracted with ethyl acetate (3×5 mL). The combined organic layers were dried over Na₂SO₄ and concentrated to afford the title compound (240 mg, crude) as a yellow solid.

Step B. (S)-4-(4-((1R,5S)-3,8-diazabicyclo[3.2.1]octan-3-yl)-6-chloro-8-fluoro-2-(((S)-2-methylenetetrahydro-1H-pyrrolizin-7a(5H)-yl)methoxy)quinazolin-7-yl)-2-amino-7-fluorobenzo[b]thiophene-3-carbonitrile: To a solution of tert-butyl (1R,5S)-3-(7-((S)-2-((tert-butoxycarbonyl)amino)-3-cyano-7-fluorobenzo[b]thiophen-4-yl)-6-chloro-8-fluoro-2-(((S)-2-methylenetetrahydro-1H-pyrrolizin-7a(5H)-yl)methoxy)quinazolin-4-yl)-3,8-diazabicyclo[3.2.1]octane-8-carboxylate (300 mg, 1.0 equiv) in DCM (3 mL) was added TFA (4.61 g, 112 equiv). The reaction was stirred at 20° C. for 0.5 hours. The mixture was concentrated in vacuum and basified with saturated aqueous NaHCO₃(5 mL). The aqueous phase was extracted with ethyl acetate (3×4 mL). The combined organic layers were dried over Na₂SO₄, concentrated and purified by prep-HPLC [column: Phenomenex luna C18 150×25 mm×10 μm; A: water (FA), B: ACN; B %: 12%-42% over 9 min] to afford the title compound (118 mg, 51% yield) as off-white solid; ¹H NMR (400 MHz, methanol-d4) δ=7.85 (d, J=1.6 Hz, 1H), 7.20 (dd, J=5.2, 8.4 Hz, 1H), 7.03 (dd, J=8.4, 9.2 Hz, 1H), 4.97 (br s, 2H), 4.55-4.38 (m, 2H), 4.28-4.19 (m, 2H), 3.74-3.53 (m, 5H), 3.29 (br s, 1H), 3.17-3.09 (m, 1H), 2.82-2.75 (m, 1H), 2.74-2.66 (m, 1H), 2.50-2.42 (m, 1H), 2.20-2.09 (m, 1H), 2.03-1.88 (m, 2H), 1.87-1.75 (m, 5H); LCMS (ESI, M+1): m/z=634.3.

### Example 184

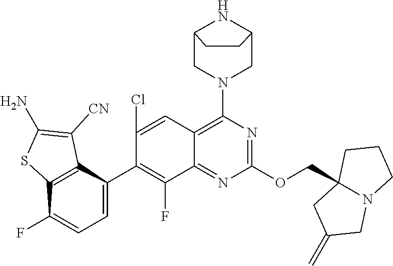

### (R)-4-(4-((1R,5S)-3,8-diazabicyclo[3.2.1]octan-3-yl)-6-chloro-8-fluoro-2-(((S)-2-methylenetetrahydro-1H-pyrrolizin-7a(5H)-yl)methoxy)quinazolin-7-yl)-2-amino-7-fluorobenzo[b]thiophene-3-carbonitrile

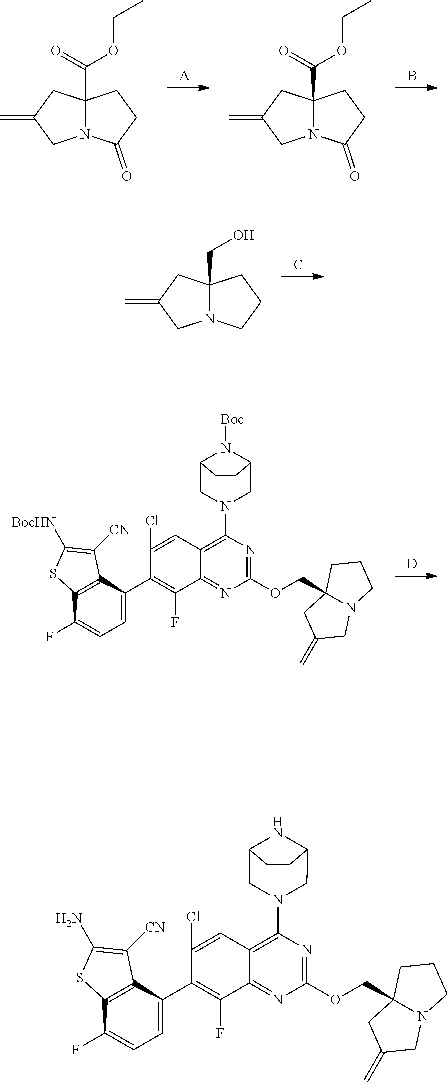

Step A. ethyl (S)-2-methylene-5-oxotetrahydro-1H-pyrrolizine-7a(5H)-carboxylate: Ethyl 2-methylene-5-oxotetrahydro-1H-pyrrolizine-7a(5H)-carboxylate (10 g) was separated by SFC [column: DAICEL CHIRALPAK IK(250 mm×50 mm, 10 um);mobile phase: CO₂-i-PrOH(0.1% NH₃H₂O);B %:0%, isocratic elution mode] to afford the title compound (4.4 g, 41% yield) as light yellow gum; LCMS (ESI, M+1): m/z=210.1.

Step B. (S)-(2-methylenetetrahydro-1H-pyrrolizin-7a(5H)-yl)methanol: To a solution of ethyl (S)-2-methylene-5-oxotetrahydro-1H-pyrrolizine-7a(5H)-carboxylate (1.00 g, 1.0 equiv) in THF (10.0 mL) was slowly added DIBAL-H (1 M, 10 equiv) at 0° C. The reaction was stirred at 0° C. for 3.5 hours. The mixture was quenched with water (2.4 mL), then 15% NaOH (2.4 mL) was added into the mixture and water (7.2 mL) was added. The mixture was dried over anhydrous sodium sulfate, concentrated under vacuum. The mixture was purified with column chromatography (SiO₂, petroleum ether/ethyl acetate 100/1 to 0/1) to afford the title compound (200 mg, 24% yeild) as yellow oil; ¹H NMR (400 MHz, CHLOROFORM-d) δ=4.96-4.86 (m, 2H), 3.63 (br d, J=14.4 Hz, 1H), 3.33-3.21 (m, 3H), 3.14-3.03 (m, 1H), 2.66 (td, J=7.2, 10.4 Hz, 1H), 2.51-2.41 (m, 1H), 2.39-2.24 (m, 1H), 1.95-1.84 (m, 2H), 1.84-1.74 (m, 1H), 1.73-1.64 (m, 1H); LCMS (ESI, M+1): m/z=154.2.

Step C. tert-butyl 3-(7-((R)-2-((tert-butoxycarbonyl)amino)-3-cyano-7-fluorobenzo[b]thiophen-4-yl)-6-chloro-8-fluoro-2-(((S)-2-methylenetetrahydro-1H-pyrrolizin-7a(5H)-yl)methoxy)quinazolin-4-yl)-3,8-diazabicyclo[3.2.1]octane-8-carboxylate: To a solution of (S)-(2-methylenetetrahydro-1H-pyrrolizin-7a(5H)-yl)methanol (39.3 mg, 1.2 equiv) in THE (2.00 mL) was added NaH (25.7 mg, 60% purity, 3.0 equiv) at 0° C. The mixture was stirred at 0° C. for 0.5 hours. tert-butyl (1R,5S)-3-(7-((R)-2-((tert-butoxycarbonyl)amino)-3-cyano-7-fluorobenzo[b]thiophen-4-yl)-6-chloro-2,8-difluoroquinazolin-4-yl)-3,8-diazabicyclo[3.2.1]octane-8-carboxylate (150 mg, 1.0 equiv) was added into the mixture and the reaction was stirred at 10° C. for 0.5 hours. The mixture was quenched with water(30 mL) and extracted with ethyl acetate (2×15 mL). The combined organic layers were dried over anhydrous sodium sulfate, concentrated under vacuum to afford the title compound (290 mg, crude) as yellow solid; LCMS (ESI, M+1, M+3): m/z=834.2, 836.2.

Step D. (4R)-4-(4-(3,8-diazabicyclo[3.2.1]octan-3-yl)-6-chloro-8-fluoro-2-(((S)-2-methylenetetrahydro-1H-pyrrolizin-7a(5H)-yl)methoxy)quinazolin-7-yl)-2-amino-7-fluorobenzo[b]thiophene-3-carbonitrile: To a solution of tert-butyl 3-(7-((R)-2-((tert-butoxycarbonyl)amino)-3-cyano-7-fluorobenzo[b]thiophen-4-yl)-6-chloro-8-fluoro-2-(((S)-2-methylenetetrahydro-1H-pyrrolizin-7a(5H)-yl)methoxy)quinazolin-4-yl)-3,8-diazabicyclo[3.2.1]octane-8-carboxylate (280 mg, 1.0 equiv) in DCM (5.00 mL) was added TFA (7.68 g, 5 mL). The reaction was stirred at 20° C. for 1 hour. The mixture was concentrated under vacuum and basified by saturated NaHCO₃ solution (50 mL) and extracted with ethyl acetate (2×25 mL) The combined organic layers were washed with brine (30 mL), dried over anhydrous sodium sulfate, concentrated and purified with prep-HPLC [column: Phenomenex luna C18 150×25 mm×10 μm; A: water(FA), B: ACN; B %: 12%-42% B over 9 min] followed by prep-HPLC [column: Waters Xbridge 150×25 mm×5 μm; A: water(NH₄HCO₃), B: ACN; B %:34%-64% over 9 min] to afford the title compound (63.0 mg, 29% yield) as white solid; ¹H NMR (400 MHz, METHANOL-d4) δ=7.85 (d, J=1.2 Hz, 1H), 7.20 (dd, J=5.2, 8.4 Hz, 1H), 7.03 (t, J=8.8 Hz, 1H), 4.97 (br s, 2H), 4.51 (br d, J=12.0 Hz, 1H), 4.46-4.38 (m, 1H), 4.29-4.18 (m, 2H), 3.76-3.54 (m, 5H), 3.34-3.32 (m, 1H), 3.18-3.09 (m, 1H), 2.78 (br d, J=16.0 Hz, 1H), 2.71 (td, J=7.2, 10.2 Hz, 1H), 2.47 (br d, J=15.6 Hz, 1H), 2.19-2.10 (m, 1H), 2.01-1.93 (m, 1H), 1.93-1.77 (m, 6H); LCMS (ESI, M+1): m/z=634.2.

### Example 185

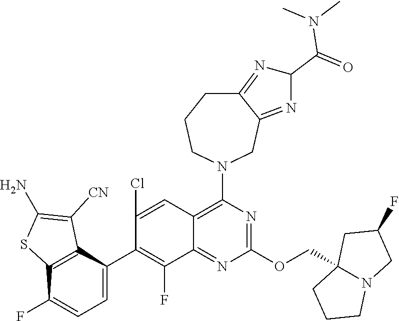

### 5-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide

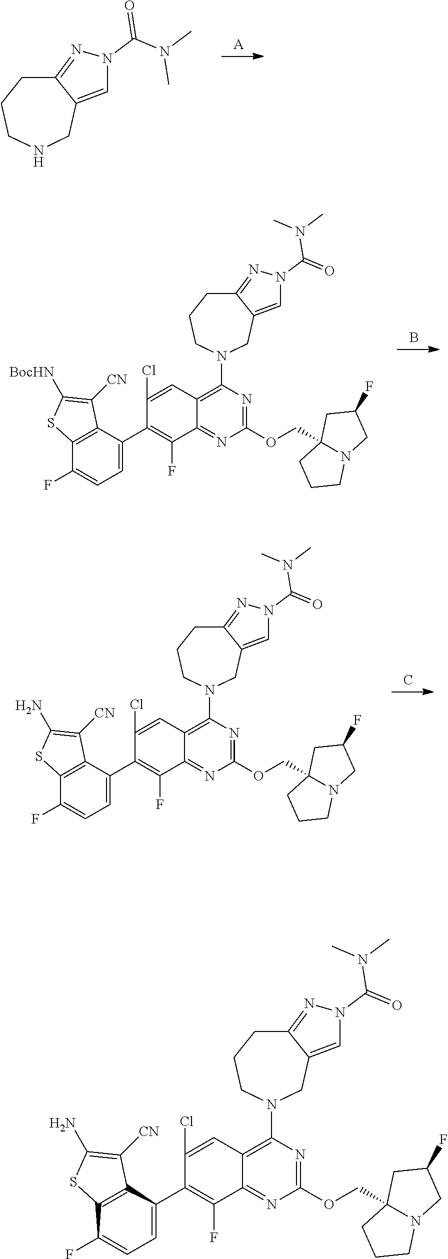

Step A. tert-butyl (4-(6-chloro-4-(2-(dimethylcarbamoyl)-2,6,7,8-tetrahydropyrazolo[4,3-c]azepin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (400 mg 1.0 equiv) and TEA (188 mg, 3 equiv) in DMSO (4 mL) was added PYBOP (483 mg, 1.5 equiv). The reaction was stirred at 25° C. for 0.5 hours. N,N-dimethyl-5,6,7,8-tetrahydropyrazolo[4,3-c]azepine-2(4H)-carboxamide (258 mg, 2.0 equiv) was added to the mixture. The reaction was stirred at 25° C. for 1.5 hours. The mixture was diluted by water (10 mL) and extracted with ethyl acetate (3×10 mL). The combined organic phase was dried over anhydrous sodium sulfate, concentrated and purified by reversed phase flash chromatography [C18, 0.1% formic acid] to afford the title compound (480 mg, 83% yield) as yellow solid

¹H NMR (400 MHz, CHLOROFORM-d) δ=8.09 (s, 1H), 7.85 (s 1H), 7.33-7.28 (m, 1H), 7.17-7.12 (m, 1H), 5.39-5.18 (m, 2H), 4.90-4.74 (m, 2H), 4.21 (s, 4H), 3.24 (br s, 6H), 3.06-2.94 (m, 4H), 2.28-2.12 (m, 6H), 1.96-1.88 (m, 4H), 1.57 (s, 9H).

Step B. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: To a solution of tert-butyl (4-(6-chloro-4-(2-(dimethylcarbamoyl)-2,6,7,8-tetrahydropyrazolo[4,3-c]azepin-5(4H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (400 mg, 1.0 equiv) in DCM (8.0 mL) was added TFA (4.0 mL, 112 equiv). The reaction was stirred at 25° C. for 0.5 hours. The mixture was concentrated and purified by Prep-HPLC [(column: Phenomenex luna C18 150×25 mm×10 um; mobile phase: [water (FA)−ACN]; gradient: 26%-66% B over 10 min] to afford the title compound (210 mg, 53.8% yield) as white solid; LCMS (ESI, M+1): m/z=736.2.

Step C. 5-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: The racemic of 5-((R)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide (210 mg) was separated by SFC (column: DAICEL CHIRALPAK IC(250 mm×30 mm,10 um);mobile phase: [CO₂-ACN/EtOH(0.1% NH₃.H₂O)]; B %:45%, isocratic elution mode) followed by reversed-phase HPLC (column: Waters xbridge 150×25 mm 10 um; mobile phase: [water(NH₄HCO₃)−ACN];gradient:48%-78% B over 10 min) to afford the title compound (27.0 mg, 12.7% yield) as a white solid

¹H NMR (400 MHz, MeOH-d4) δ=8.16 (s, 1H), 7.99 (d, J=1.2 Hz, 1H), 7.20 (dd, J=5.2, 8.4 Hz, 1H), 7.06-7.00 (m, 1H), 5.39-5.22 (m, 1H), 4.96 (s, 2H), 4.32-4.24 (m, 3H), 4.21-4.15 (m, 1H), 3.39-3.34 (m, 1H), 3.24 (br d, J=1.2 Hz, 2H), 3.19 (s, 6H), 3.04-2.95 (m, 3H), 2.30-2.12 (m, 5H), 2.02-1.96 (m, 2H), 1.93-1.85 (m, 1H); LCMS (ESI, M+1): m/z=736.2.

### Example 186

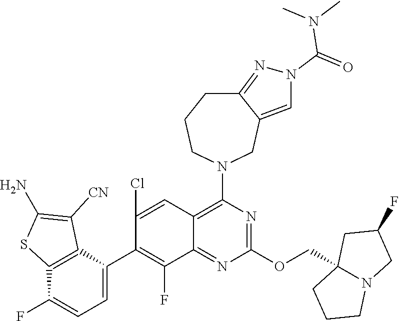

### 5-((S)-7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide

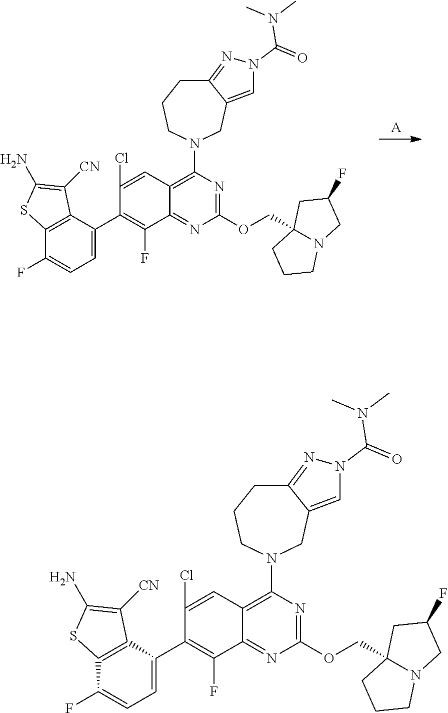

Step A. 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide: The racemic of 5-(7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydropyrazolo[4,3-c]azepine-2(4H)-carboxamide (120 mg) was separated by SFC (column: DAICEL CHIRALPAK IC(250 mm×30 mm,10 um);mobile phase: [CO₂-ACN/EtOH(0.1% NH₃.H₂O)]; B %:45%, isocratic elution mode) followed by reversed-phase HPLC (column: Waters xbridge 150×25 mm 10 um; mobile phase: [water(NH₄HCO₃)−ACN]; gradient:47%-77% B over 10 min) to afford the tittle compound (48.5 mg, 83.9% yield) as a white solid.

¹H NMR (400 MHz, MeOH-d4) δ=8.16 (s, 1H), 7.99 (d, J=1.2 Hz, 1H), 7.20 (dd, J=5.2, 8.4 Hz, 1H), 7.03 (t, J=8.8 Hz, 1H), 5.39-5.21 (m, 1H), 4.96 (br d, J=3.6 Hz, 2H), 4.32-4.23 (m, 3H), 4.20-4.15 (m, 1H), 3.43-3.35 (m, 1H), 3.27-3.22 (m, 2H), 3.19 (br s, 6H), 3.02-2.96 (m, 3H), 2.30-2.12 (m, 5H), 2.05-1.96 (m, 2H), 1.92-1.83 (m, 1H); LCMS (ESI, M+1): m/z=736.2

### Example 187

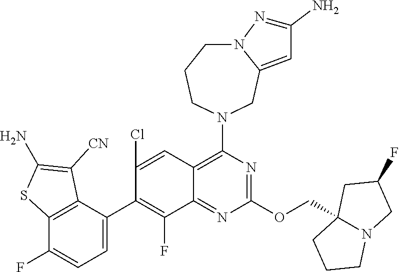 [SMILES: N#Cc1c(N)sc2c(F)ccc(-c3c(Cl)cc4c(N5CCCn6nc(N)cc6C5)nc(OC[C@@]56CCCN5C[C@H](F)C6)nc4c3F)c12]

### 2-amino-4-(4-(2-amino-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

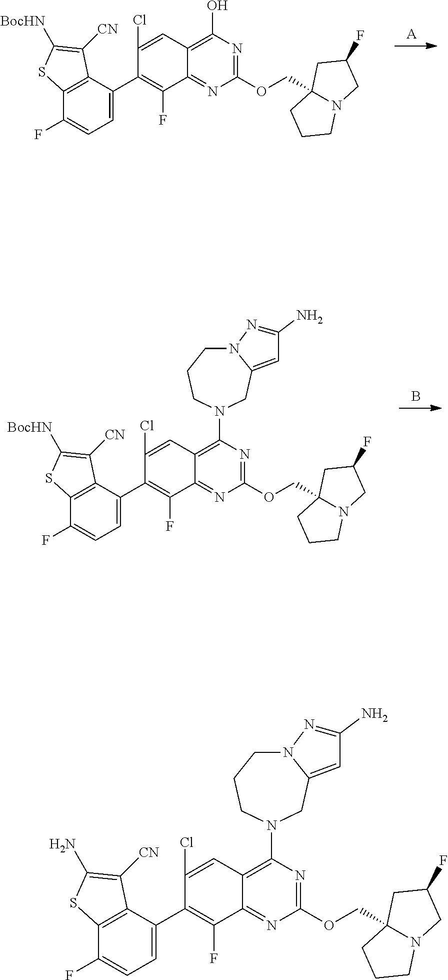

Step A. tert-butyl (4-(4-(2-amino-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl)(4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 equiv) and TEA (23.5 mg, 3.0 equiv) in DMSO (0.5 mL) was added PyBOP (60.4 mg, 1.5 equiv) in one portion under N₂. After stirring at 20° C. for 20 minutes, 5,6,7,8-tetrahydro-4H-pyrazolo[1,5-a][1,4]diazepin-2-amine (23.6 mg, 2.0 equiv) was added. The reaction was stirred at 20° C. for 1 hour. The mixture was diluted with water (2 mL) and extracted with ethyl acetate (3×3 mL). The combined organic layers were washed with brine (3 mL), dried over anhydrous sodium sulfate, filtered, concentrated and purified by reversed-phase [water (0.1% formic acid)/acetonitrile)] to afford the title compound (60.0 mg, 95% yield) as yellow solid; LCMS (ESI, M+1): m/z=780.4.

Step B. 2-amino-4-(4-(2-amino-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a mixture of tert-butyl (4-(4-(2-amino-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (60.0 mg, 1.0 equiv) in DCM (1 mL) was added TFA (768 mg, 88 equiv) in one portion under N₂. The reaction was stirred at 20° C. for 1 hour. The mixture was concentrated, adjusted to pH=8 with saturated NaHCO₃ aqueous solution and extracted with DCM (3×15 mL) The combined organic layers were washed with brine (15 mL), dried over anhydrous sodium sulfate, filtered, concentrated and purified by prep-HPLC [Phenomenex luna C18 150×25 mm×10 um; A: water (FA); B: ACN, B %: 18%-48% over 10 min] to afford the title compound (18.6 mg, 34.6% yield) as white solid; ¹H NMR (400 MHz, DMSO-d₆) δ=8.16-8.07 (m, 2H), 8.03 (s, 1H), 7.29-7.23 (m, 1H), 7.19-7.12 (m, 1H), 5.54 (s, 1H), 5.41-5.22 (m, 1H), 4.93-4.72 (m, 2H), 4.15 (br d, J=3.2 Hz, 3H), 4.12-3.98 (m, 3H), 3.25-3.02 (m, 3H), 2.96-2.81 (m, 1H), 2.18 (br d, J=5.2 Hz, 3H), 2.13-2.03 (m, 2H), 1.93-1.75 (m, 3H); LCMS (ESI, M+1): m/z=680.2.

### Example 188

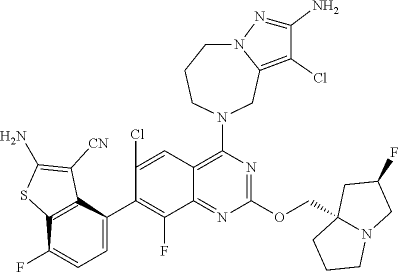

### 2-amino-4-((R)-4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

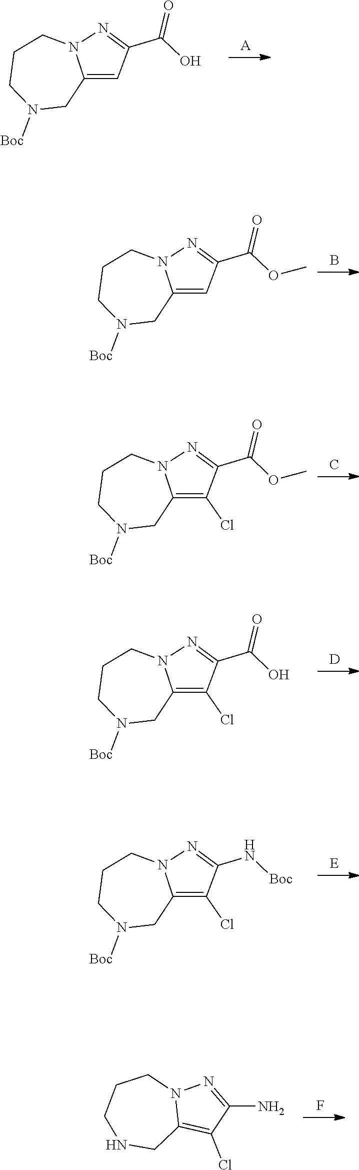

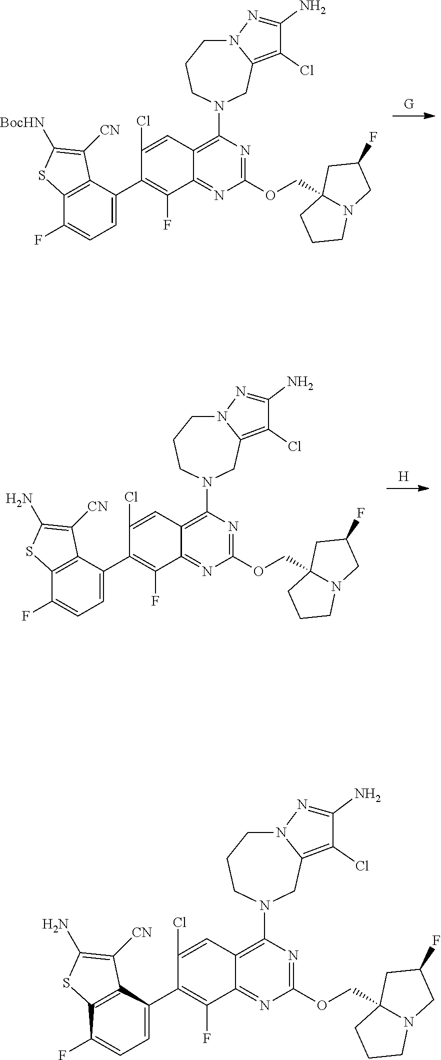

Step A. 5-(tert-butyl) 2-methyl 7,8-dihydro-4H-pyrazolo[1,5-a][1,4]diazepine-2,5(6H)-dicarboxylate: To a solution of 5-(tert-butoxycarbonyl)-5,6,7,8-tetrahydro-4H-pyrazolo[1,5-a][1,4]diazepine-2-carboxylic acid (3.80 g, 1.0 equiv) in dichloromethane (30.0 mL) and methyl alcohol (15.0 mL) was added TMSCHN₂ (2 M, 20.3 mL, 3.0 equiv) at 0° C. dropwise. The reaction was stirred at 0° C. for 1.0 hour. The mixture was concentrated, diluted with water (50.0 mL) and extracted with dichloromethane (3×50.0 mL). The combined organic phase was washed with brine (20.0 mL), dried over anhydrous sodium sulfate, filtered and concentrated to afford the title compound (4.70 g, crude) as a white solid. LCMS (ESI, M+1): m/z=296.0.

Step B. 5-(tert-butyl) 2-methyl 3-chloro-7,8-dihydro-4H-pyrazolo[1,5-a][1,4]diazepine-2,5(6H)-dicarboxylate: To a solution of 5-(tert-butyl) 2-methyl 7,8-dihydro-4H-pyrazolo[1,5-a][1,4]diazepine-2,5(6H)-dicarboxylate (3.70 g, 1.0 equiv) in acetic acid (40.0 mL) was added NCS (2.01 g, 1.2 equiv). The reaction was stirred at 80° C. for 0.5 hours. The mixture was concentrated, diluted with saturated sodium bicarbonate (30.0 mL) and extracted with ethyl acetate (3×50.0 mL). The combined organic layers were dried over anhydrous sodium sulfate, filtered and concentrated to afford the title compound (5.00 g, crude) as a yellow oil; LCMS (ESI, M+1): m/z=330.1.

Step C. 5-(tert-butoxycarbonyl)-3-chloro-5,6,7,8-tetrahydro-4H-pyrazolo[1,5-a][1,4]diazepine-2-carboxylic acid: To a solution of 5-(tert-butyl) 2-methyl 3-chloro-7,8-dihydro-4H-pyrazolo[1,5-a][1,4]diazepine-2,5(6H)-dicarboxylate (4.5 g, 1.0 equiv) in methyl alcohol (20.0 mL) was added a solution of LiOH.H₂O (1.72 g, 3.0 equiv) in water (20.0 mL). The reaction was stirred at 20° C. for 2 hours. The mixture was washed with EtOAc (3×10.0 mL) to remove the impurities. The aqueous phase was cooled to 0° C., acidified with hydrochloric acid (1 M, 10 mL) and extracted with ethyl acetate (3×50.0 mL). The combined organic phase was washed with brine (3×100 mL), dried over anhydrous sodium sulfate, filtered and concentrated to afford the title compound (4.10 g, 94% yield) as a white solid; LCMS (ESI, M+1): m/z=316.0.

Step D. tert-butyl 2-((tert-butoxycarbonyl)amino)-3-chloro-7,8-dihydro-4H-pyrazolo[1,5-a][1,4]diazepine-5(6H)-carboxylate: A mixture of 5-(tert-butoxycarbonyl)-3-chloro-5,6,7,8-tetrahydro-4H-pyrazolo[1,5-a][1,4]diazepine-2-carboxylic acid (3.6 g, 1.0 equiv), DPPA (4.71 g, 1.5 equiv), TEA (3.46 g, 3.0 equiv) and 4 Å molecular sieve (100 mg, 1.0 equiv) in t-BuOH (40.0 mL) was stirred at 100° C. for 3 hours under nitrogen atmosphere. The mixture was filtered, concentrated and purified by prep-HPLC [Phenomenex luna C18 250×70 mm, 15 um; A: water (FA), B: ACN; B %: 45%-70% over 18 min] to afford the title compound (4.00 mg, 76% yield) as a white solid; LCMS (ESI, M+1): m/z=387.1.

Step E. 3-chloro-5,6,7,8-tetrahydro-4H-pyrazolo[1,5-a][1,4]diazepin-2-amine: A solution of tert-butyl 2-((tert-butoxycarbonyl)amino)-3-chloro-7,8-dihydro-4H-pyrazolo[1,5-a][1,4]diazepine-5(6H)-carboxylate (500 mg, 1.0 equiv) in HCl.MeOH (4 M, 10.2 mL, 46 equiv) was stirred at 20° C. for 1 hours. The mixture was concentrated to afford the title compound (500 mg, crude) as a white oil; LCMS (ESI, M+1): m/z=187.0.

Step F. tert-butyl (4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-(6-chloro-8-fluoro-2-(((2R,7aS)-2-fluorotetrahydro-1H-pyrrolizin-7a(5H)-yl)methoxy)-5-hydroxyquinazolin-7-yl)-3-cyano-7-fluorobenzo[b]thiophen-2-yl)carbamate (500 mg, 1.0 equiv), PYBOP (604 mg, 1.5 equiv) in DMSO (0.5 mL) was added TEA (235 mg, 3.0 equiv). The reaction was stirred at 20° C. for 0.5 hours. 3-chloro-5,6,7,8-tetrahydro-4H-pyrazolo[1,5-a][1,4]diazepin-2-amine (259 mg, 1.5 equiv, HCl) was added into the mixture. The reaction mixture was stirred at 20° C. for 0.5 hours. The mixture was filtered and purified by prep-HPLC [Waters xbridge 150×25 mm 10 um; A: water (NH₄HCO₃), B: ACN; B %:56%-86% over 14 min] to afford the title compound (300 mg, 47% yield) as a yellow solid; LCMS (ESI, M+1, M+3): m/z=814.1, 816.2.

Step G. 2-amino-4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (250 mg, 1.0 equiv) in DCM (2.0 mL) was added TFA (3.07 g, 88 equiv) at 0° C. The reaction was stirred at 20° C. for 1 hours. The mixture was concentrated, diluted with saturated sodium bicarbonate (2.0 mL) and extracted with ethyl acetate (5 mL×3). The combined organic layers were dried over by anhydrous sodium sulfate, filtered, concentrated and purified by prep-HPLC [Waters xbridge 150×25 mm 10 um; A: water (NH₄HCO₃), B: ACN; B %: 42%-72% over 14 min] to afford the title compound (200 mg, 91.2% yield) as a white solid; LCMS (ESI, M+1): m/z=714.0.

Step H. 2-amino-4-((R)-4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: The racemic of 2-amino-4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (200 mg, 1.0 equiv) was separated by SFC (DAICEL CHIRALCEL OX (250 mm×30 mm, 10 um); mobile phase: [CO₂-MeOH (0.1% NH₃H₂O)]; B %: 65%, isocratic elution mode) to afford the title compound (72.6 mg, 35% yield) as white solid and (79.7 mg, 39% yield) as off-white solid; ¹H NMR (400 MHz, METHANOL-d₄) δ=7.88 (s, 1H), 7.20 (dd, J=5.2, 8.4 Hz, 1H), 7.03 (t, J=8.8 Hz, 1H), 5.42-5.22 (m, 1H), 4.93 (br d, J=9.2 Hz, 2H), 4.30-4.19 (m, 4H), 4.18 (br s, 2H), 3.28-3.08 (m, 3H), 3.05-2.93 (m, 1H), 2.42-2.09 (m, 5H), 2.04-1.85 (m, 3H); LCMS (ESI, M+1): m/z=714.2.

### Example 189

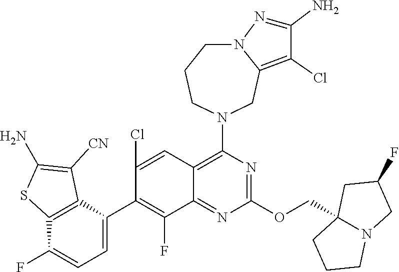

### 2-amino-4-((S)-4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

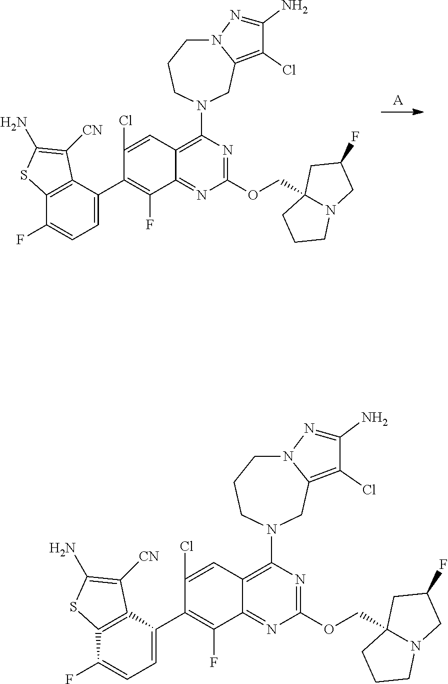

Step A. 2-amino-4-((R)-4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: The racemic of 2-amino-4-(4-(2-amino-3-chloro-7,8-dihydro-4H-pyrazolo[1,5-a][1,4]diazepin-5(6H)-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile (200 mg, 1.0 equiv) was separated by SFC (DAICEL CHIRALCEL OX (250 mm×30 mm, 10 um); mobile phase: [CO₂-MeOH (0.1% NH₃H₂O)]; B %: 65%, isocratic elution mode) to afford the title compound (79.7 mg, 39% yield) as off-white solid;

¹H NMR (400 MHz, METHANOL-d4) δ=7.89 (d, J=1.2 Hz, 1H), 7.20 (dd, J=5.2, 8.4 Hz, 1H), 7.03 (t, J=8.8 Hz, 1H), 5.40-5.21 (m, 1H), 5.00-4.91 (m, 2H), 4.28-4.20 (m, 4H), 4.15 (br d, J=8.9 Hz, 2H), 3.30-3.10 (m, 3H), 3.06-2.96 (m, 1H), 2.42-2.09 (m, 5H), 2.04-1.84 (m, 3H); LCMS (ESI, M+1): m/z=714.2.

### Example 190

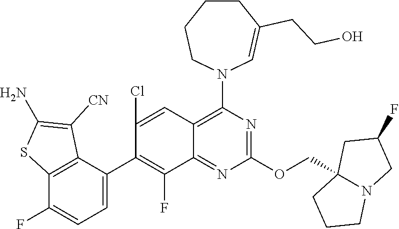 [SMILES: N#Cc1c(N)sc2c(F)ccc(-c3c(Cl)cc4c(N5C=C(CCO)CCCC5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(6-(2-hydroxyethyl)-2,3,4,5-tetrahydro-1H-azepin-1-yl)quinazolin-7-yl)-7-fluorobenzo[b]thiophene-3-carbonitrile

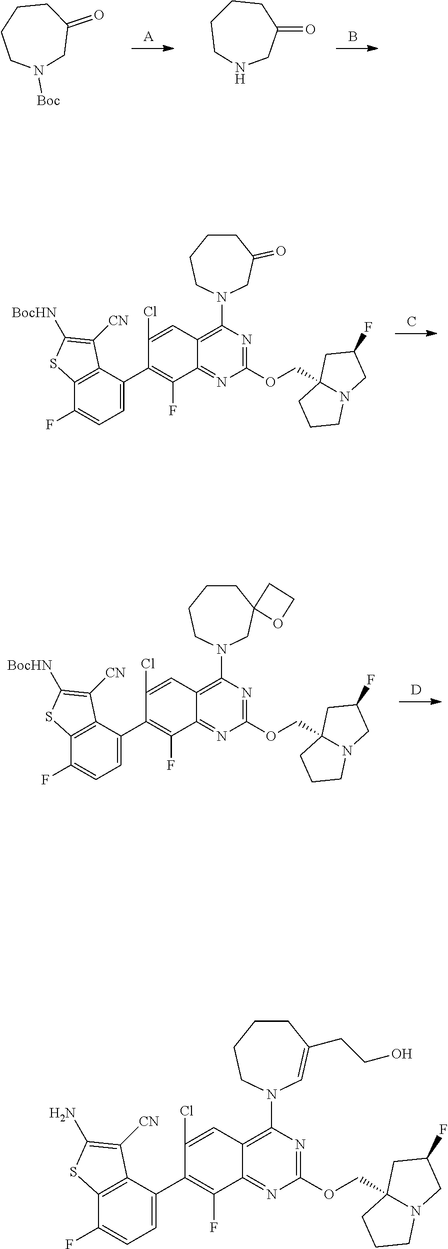

Step A. azepan-3-one: To a solution of tert-butyl 3-oxoazepane-1-carboxylate (200 mg, 1 equiv) in DCM (0.6 mL) was added TFA (307 mg, 2.87 equiv). The reaction was stirred at 25° C. for 0.5 hours. The mixture was concentrated in vacuum to afford the title compound (400 mg, crude) as yellow oil.

Step B. tert-butyl (4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate: To a solution of azepan-3-one (105 mg, 2 equiv, TFA) and tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (150 mg, 1 equiv) in DMSO (1.00 mL) were added TEA (70.5 mg, 3 equiv) and PYBOP (181 mg, 1.5 equiv). The reaction was stirred at 25° C. for 2 hours. The mixture was diluted with water (15 mL) and extracted with EtOAc (5.0 mL×3). The combined organic layers were washed with brine (5.0 mL×2), dried over Na₂SO₄, filtered, concentrated and purified by prep-TLC (SiO₂, DCM: MeOH=10:1) followed by reversed phase flash (0.1% FA condition) to afford the title compound (50 mg, 29% yield) as a yellow solid; LCMS (ESI, M+1): m/z=741.4.

Step C. tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate: To a solution of trimethylsulfoxonium iodide (44.5 mg, 3 equiv) in t-BuOH (0.5 mL) was added t-BuOK (1 M, 202 μL, 3 equiv). The reaction was stirred at 50° C. for 2 hours. To the mixture was added a solution of tert-butyl (4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate: To a solution of azepan-3-one (50.0 mg, 1 equiv) in t-BuOH (1.00 mL). The reaction was stirred at 50° C. for 5 hours. The mixture was concentrated in vacuum and purified by prep-TLC (DCM: MeOH=10:1) to afford the title compound (10 mg, 12.3 μmol, 18% yield) as a yellow solid; LCMS (ESI, M+1): m/z=769.4.

Step D. 2-amino-4-(6-chloro-8-fluoro-2-(((2R,7aS)-2-fluorotetrahydro-1H-pyrrolizin-7a(5H)-yl)methoxy)-4-(6-(2-hydroxyethyl)-2,3,4,5-tetrahydro-1H-azepin-1-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(1-oxa-6-azaspiro[3.6]decan-6-yl)quinazolin-7-yl)-3-cyano-7-fluorobenzo[b]thiophen-2-yl)carbamate (8.00 mg, 1 equiv) in DCM (1.00 mL) was added TFA (614 mg, 518 equiv). The reaction was stirred at 0° C. for 3 hours. To the mixture was adjusted pH to 7-8 with TEA at 0° C., concentrated in vacuum and purified by prep-HPLC (column: Phenomenex C18 150×25 mm×10 um; mobile phase: [water(NH₄.HCO₃)−ACN]; gradient: 45%-75% B over 10 min) to afford the title compound (1.51 mg, 21% yield) as yellow solid; ¹H NMR (400 MHz, dimethyl sulfoxide-d6) 6=8.51 (s, 1H), 8.11 (s, 2H), 7.26-7.20 (m, 1H), 7.18-7.11 (m, 1H), 6.56 (s, 1H), 5.50-5.16 (m, 1H), 4.64 (t, J=5.2 Hz, 1H), 4.40-4.27 (m, 1H), 4.27-4.03 (m, 1H), 4.03-3.93 (m, 1H), 3.62 (q, J=6.0 Hz, 2H), 3.19-2.74 (m, 4H), 2.40-2.31 (m, 3H), 2.28 (br t, J=6.4 Hz, 2H), 2.20-2.01 (m, 3H), 1.92-1.86 (m, 3H), 1.85-1.75 (m, 2H), 1.74-1.65 (m, 2H); LCMS (ESI, M+1): m/z=669.2.

### Example 191

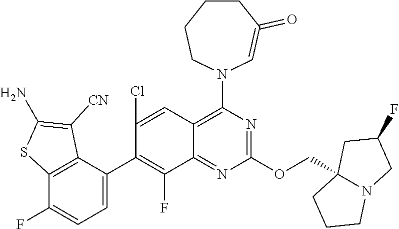

### 2-amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile

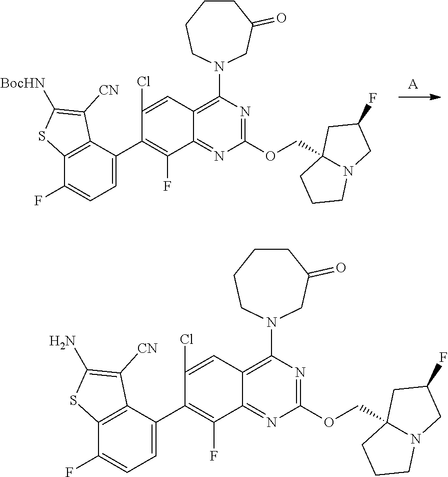

Step A. 2-amino-4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(3-oxoazepan-1-yl)quinazolin-7-yl)-3-cyano-7-fluorobenzo[b]thiophen-2-yl)carbamate (80.0 mg, 1 equiv) in DCM (1.00 mL) was added TFA (368 mg, 29.9 equiv). The reaction was stirred at 20° C. for 1 hour. The mixture was concentrated in vacuum, dissolved in MeCN, adjusted pH to 7 with NaHCO₃ and filtered. The filtrate was concentrated and purified by prep-HPLC (column: Waters xbridge 150×25 mm×10 um; mobile phase: [water(NH₄.HCO₃)−ACN]; gradient: 41%-71% B over 10 min) to afford the title compound (20.0 mg, 28% yield) as white solid; ¹H NMR (400 MHz, dimethyl sulfoxide-d6) 6=8.11 (s, 2H), 8.04 (s, 1H), 7.26 (dd, J=5.2, 8.4 Hz, 1H), 7.20-7.10 (m, 1H), 5.40-5.13 (m, 1H), 4.56-4.38 (m, 2H), 4.26-4.09 (m, 2H), 4.09-4.04 (m, 1H), 4.03-3.96 (m, 1H), 3.15-3.04 (m, 2H), 3.01 (br s, 1H), 2.87-2.77 (m, 1H), 2.45 (br d, J=3.6 Hz, 2H), 2.18-2.08 (m, 1H), 2.04 (br s, 1H), 1.98 (br s, 1H), 1.92 (br d, J=5.2 Hz, 2H), 1.82 (br d, J=14.4 Hz, 2H), 1.76 (br d, J=5.6 Hz, 3H); LCMS (ESI, M+1): m/z=641.2.

### Example 192

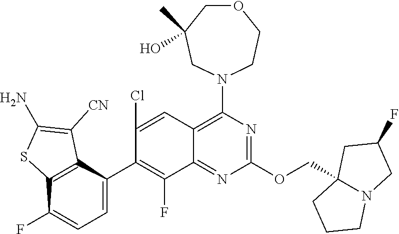

### 2-amino-4-((R)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile

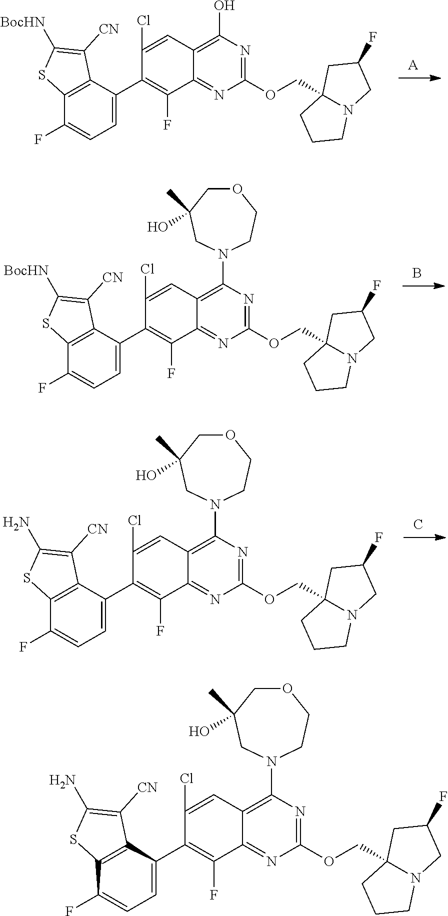

Step A. tert-butyl (4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (500 mg, 1 equiv) and (S)-6-methyl-1,4-oxazepan-6-ol (203 mg, 2 equiv) in DMSO (10.0 mL) was added TEA (235 mg, 3 equiv) and PYBOP (604 mg, 1.5 equiv). The reaction was diluted with water (50 mL) and extracted with ethyl acetate (3×20.0 mL). The combined organic layers were washed with brine (2×20.0 mL), dried over Na₂SO₄, filtered, concentrated under reduced pressure and purified by reversed-phase HPLC (C18, 0.1% FA condition) to afford the title compound (370 mg, 59.8% yield) as a yellow gum; LCMS (ESI, M+1): m/z=759.2.

Step B. 2-amino-4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-3-cyano-7-fluorobenzo[b]thiophen-2-yl)carbamate (330 mg, 1 equiv) in DCM (2.40 mL) was added TFA (1.23 g, 24.8 equiv). The reaction was stirred at 25° C. for 1 hour. The mixture was concentrated in vacuum, dissolved in MeCN (2.00 mL), adjusted pH to 7 with NaHCO₃, filtered, concentrated and purified by prep-HPLC (column: Waters Xbridge BEH C18 150×25 mm×5 um; mobile phase: [water (NH₄.HCO₃)−ACN]; gradient: 42%-72% B over 10 min) to afford the title compound (140 mg, 48.9% yield) as a white solid.

Step C. 2-amino-4-((R)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: The racemic of 2-amino-4-((R)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile (140 mg) was separated by SFC (column: DAICEL CHIRALCEL OX (250 mm×30 mm,10 um); mobile phase: [CO₂-ACN/MeOH (0.1% NH₃.H₂O)]; B %:40%, isocratic elution mode) to afford the tittle compound (53.0 mg, 38% yield) as a white solid; ¹H NMR (400 MHz, dimethyl sulfoxide-d6) 6=8.50 (s, 1H), 8.10 (s, 2H), 7.26 (dd, J=5.4, 8.4 Hz, 1H), 7.19-7.11 (m, 1H), 5.40-5.15 (m, 2H), 4.31-4.15 (m, 1H), 4.14-4.05 (m, 2H), 4.04-3.84 (m, 4H), 3.77-3.65 (m, 1H), 3.54 (s, 2H), 3.17-3.04 (m, 2H), 3.01 (s, 1H), 2.88-2.77 (m, 1H), 2.17-2.09 (m, 1H), 2.04 (br d, J=2.0 Hz, 1H), 2.02-1.95 (m, 1H), 1.88-1.80 (m, 1H), 1.81-1.71 (m, 2H), 1.13 (s, 3H); LCMS (ESI, M+1): m/z=659.3.

### Example 193

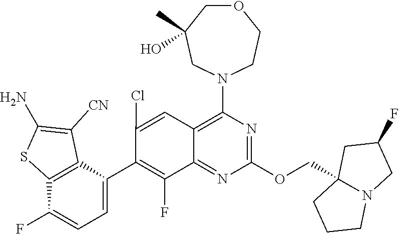

### 2-amino-4-((S)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile

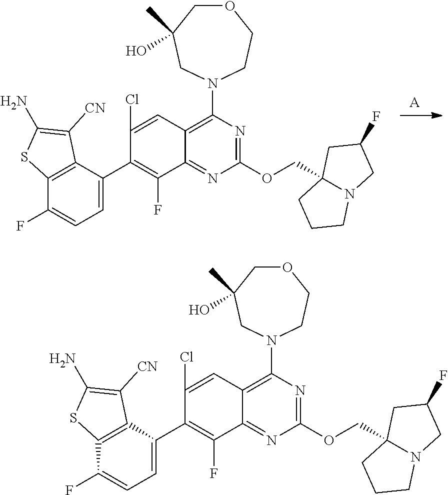

Step A. 2-amino-4-((S)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: The racemic of 2-amino-4-((R)-6-chloro-8-fluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile (140 mg) was separated by SFC (column: DAICEL CHIRALCEL OX (250 mm×30 mm,10 um); mobile phase: [CO₂-ACN/MeOH (0.1% NH₃.H₂O)]; B %:40%, isocratic elution mode) to afford the tittle compound (53.8 mg, 38% yield) as an off-white solid; ¹H NMR (400 MHz, dimethyl sulfoxide-d6) 6=8.56 (s, 1H), 8.10 (br s, 2H), 7.24 (dd, J=5.4, 8.4 Hz, 1H), 7.18-7.10 (m, 1H), 5.40-5.12 (m, 2H), 4.26 (td, J=4.8, 14.4 Hz, 1H), 4.15-4.07 (m, 2H), 4.00 (d, J=10.4 Hz, 1H), 3.95 (br t, J=5.2 Hz, 2H), 3.81 (d, J=14.8 Hz, 1H), 3.75-3.65 (m, 1H), 3.56 (s, 2H), 3.13-3.05 (m, 2H), 3.04-2.99 (m, 1H), 2.87-2.77 (m, 1H), 2.18-2.09 (m, 1H), 2.08-1.97 (m, 2H), 1.86-1.72 (m, 3H), 1.15 (s, 3H); LCMS (ESI, M+1): m/z=659.0.

### Example 194

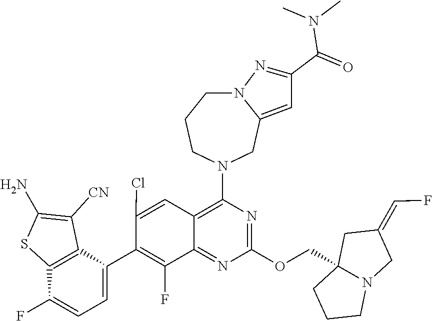

### 5-((S)-7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

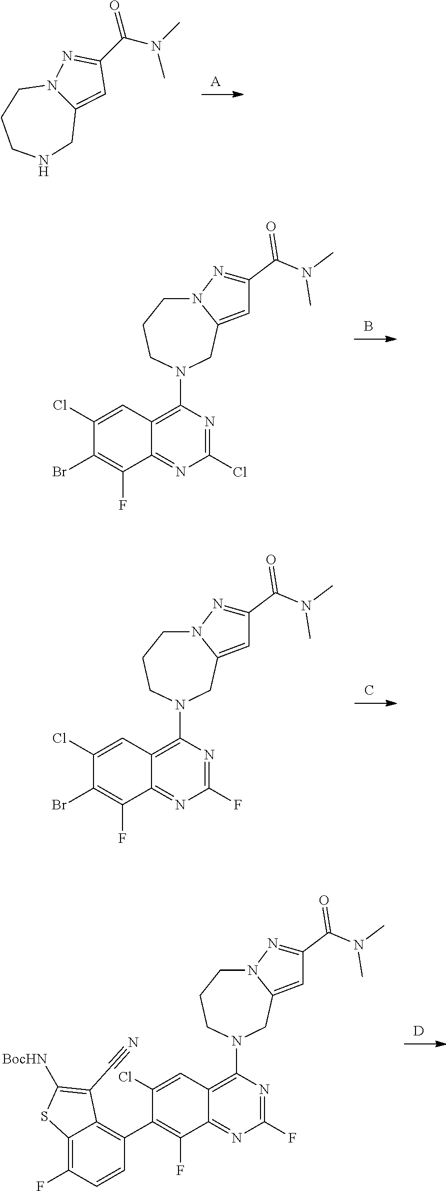

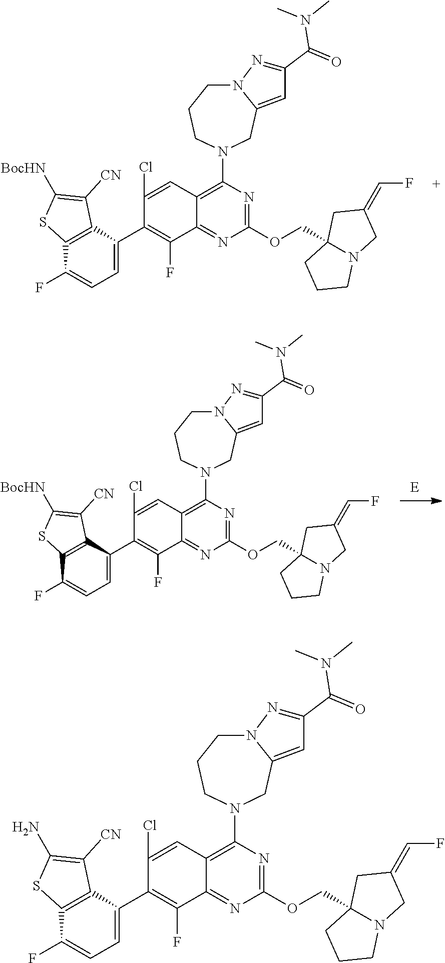

Step A. 5-(7-bromo-2,6-dichloro-8-fluoroquinazolin-4-yl)-N,N-dimethyl-5,6,7,8-tetrahydro-4H-pyrazolo[1,5-a][1,4]diazepine-2-carboxamide. To a solution of 7-bromo-2,4,6-trichloro-8-fluoro-quinazoline (1.0 g, 1.0 equiv) in DCM (10 mL) were added DIEA (1.17 g, 3.0 equiv) and N,N-dimethyl-5,6,7,8-tetrahydro-4H-pyrazolo[1,5-a][1,4]diazepine-2-carboxamide (662 mg, 1.0 equiv) in DCM (15 mL). The reaction was stirred at −40° C. for 1 hour. The mixture was diluted with water (10 mL) and extracted with EtOAc (2×10 mL). The combined organic layers were washed with brine (2×10 mL), dried over anhydrous sodium sulfate, concentrated to afford the title compound (1.5 g, 88% yield) as yellow solid; LCMS (ESI, M+1, M+3): m/z=501.1, 503.1.

Step B. 5-(7-bromo-6-chloro-2,8-difluoroquinazolin-4-yl)-N,N-dimethyl-5,6,7,8-tetrahydro-4H-pyrazolo[1,5-a][1,4]diazepine-2-carboxamide: To a solution of 5-(7-bromo-2,6-dichloro-8-fluoro-quinazolin-4-yl)-N,N-dimethyl-4,6,7,8-tetrahydropyrazolo[1,5-a][1,4]diazepine-2-carboxamide (600 mg, 1.0 equiv) in DMSO (6 mL) was added KF (2.08 g, 30 equiv). The reaction was stirred at 140° C. for 0.5 hours. The mixture was diluted with water (10 mL) and extracted with EtOAc (2×10 mL). The combined organic layers were washed with brine (2×10 mL), dried over anhydrous sodium sulfate, concentrated to afford the title compound (580 mg, 93% yield) as yellow solid; LCMS (ESI, M+1, M+3): m/z=485.2, 487.2

Step C. tert-butyl (4-(6-chloro-4-(2-(dimethylcarbamoyl)-7,8-dihydro-4H-pyrazolo[1,5-a][1,4]diazepin-5(6H)-yl)-2,8-difluoroquinazolin-7-yl)-3-cyano-7-fluorobenzo[b]thiophen-2-yl)carbamate: To a solution of 5-(7-bromo-6-chloro-2,8-difluoro-quinazolin-4-yl)-N,N-dimethyl-4,6,7,8-tetrahydropyrazolo[1,5-a][1,4]diazepine-2-carboxamide (300 mg, 1.0 equiv) in dioxane (3 mL) were added dichloro[bis(diphenylphosphinophenyl)ether]palladium(II) (84.9 mg, 0.2 equiv), tert-butyl (3-cyano-4-(5,5-dimethyl-1,3,2-dioxaborinan-2-yl)-7-fluorobenzo[b]thiophen-2-yl)carbamate (624 mg, 2.5 equiv) and Cs₂CO₃ (604 mg, 3.0 equiv). The reaction was stirred at 60° C. for 1 hour. The mixture was diluted with water (10 mL) and extracted with EtOAc (2×10 mL). The combined organic layers were washed with brine (2×10 mL), dried over anhydrous sodium sulfate, concentrated and purified by reversed phase flash chromatography [C18, 0.1% formic acid condition] to afford the title compound (300 mg, 70% yield) as red solid; LCMS (ESI, M+1): m/z=697.1.

Step D. tert-butyl (4-((S)-6-chloro-4-(2-(dimethylcarbamoyl)-7,8-dihydro-4H-pyrazolo[1,5-a][1,4]diazepin-5(6H)-yl)-8-fluoro-2-(((S,Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate:

To a solution of (S,Z)-(2-(fluoromethylene)tetrahydro-1H-pyrrolizin-7a(5H)-yl)methanol (381 mg, 1.0 equiv) in THE (20 mL) was added NaH (267 mg, 60% purity, 3.0 equiv) at 0° C. The mixture was stirred at 20° C. for 0.5 hours. Then tert-butyl (4-(6-chloro-4-(2-(dimethylcarbamoyl)-7,8-dihydro-4H-pyrazolo[1,5-a][1,4]diazepin-5(6H)-yl)-2,8-difluoroquinazolin-7-yl)-3-cyano-7-fluorobenzo[b]thiophen-2-yl)carbamate (1.55 g, 1.0 equiv) was added and the reaction was stirred at 20° C. for 2 hours. The mixture was quenched with water (20 mL) and layers were separated. The aqueous phase was extracted with ethyl acetate (3×20 mL). The combined organic layers were dried over Na₂SO₄ and concentrated and purified by reversed phase flash [water (0.1% FA)/acetonitrile], followed by SFC separation [column: DAICEL CHIRALPAK AD (250 mm×30 mm, 10 m); A: CO₂, B: EtOH (0.1% NH₃H₂O); B %: 45%, B %: over 120 min]. The desired fractions was collected and concentrated in vacuum to afford two isomers.

tert-butyl (4-((S)-6-chloro-4-(2-(dimethylcarbamoyl)-7,8-dihydro-4H-pyrazolo[1,5-a][1,4]diazepin-5(6H)-yl)-8-fluoro-2-(((S,Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (0.50 g, 26% yield) as yellow solid; LCMS (ESI, M+1): m/z=848.3; SFC>97.8% ee, column: Chiralcel AD-350×4.6 mm I.D., 3 μm; mobile phase: 40% EtOH (0.05% DEA) in CO₂, flow rate: 3 mL/min, detector: PDA, t_{R}: 1.258 min.

tert-butyl (4-((R)-6-chloro-4-(2-(dimethylcarbamoyl)-7,8-dihydro-4H-pyrazolo[1,5-a][1,4]diazepin-5(6H)-yl)-8-fluoro-2-(((S,Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (0.50 g, 26% yield) as yellow solid; LCMS (ESI, M+1): m/z=848.3; SFC>97.8% ee, column: Chiralcel AD-350×4.6 mm I.D., 3 μm; mobile phase: 40% EtOH (0.05% DEA) in CO₂, flow rate: 3 mL/min, detector: PDA, t_{R}: 0.752 min.

Step E. 5-((S)-7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of tert-butyl (4-((S)-6-chloro-4-(2-(dimethylcarbamoyl)-7,8-dihydro-4H-pyrazolo[1,5-a][1,4]diazepin-5(6H)-yl)-8-fluoro-2-(((S,Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (250 mg, 1.0 equiv) in DCM (2 mL) was added TFA (3.07 g, 92 equiv). The reaction was stirred at 20° C. for 0.5 hours. The mixture was concentrated in vacuum and basified with saturated aqueous NaHCO₃(3 mL). The aqueous phase was extracted with ethyl acetate (3×3 mL). The combined organic layers were dried over Na₂SO₄ and concentrated in vacuum. The residue was purified by prep-HPLC [column: Waters Xbridge Prep OBD C18 150×40 mm×10 um; A: water (NH₄HCO₃), B: ACN; 38%-68% over 15 min] to afford the title compound (110 mg, 49% yield) as off-white solid; ¹H NMR (400 MHz, methanol-d₄) δ=7.99 (d, J=1.6 Hz, 1H), 7.21 (dd, J=5.2, 8.4 Hz, 1H), 7.03 (dd, J=8.8, 9.2 Hz, 1H), 6.75-6.49 (m, 2H), 5.08 (s, 2H), 4.54 (t, J=5.2 Hz, 2H), 4.32-4.16 (m, 4H), 3.89-3.79 (m, 1H), 3.50-3.41 (m, 1H), 3.33 (s, 3H), 3.19-3.11 (m, 1H), 3.08 (s, 3H), 2.75-2.64 (m, 2H), 2.46-2.30 (m, 3H), 2.14-2.06 (m, 1H), 2.03-1.78 (m, 3H); LCMS (ESI, M+1): m/z=748.1.

### Example 195

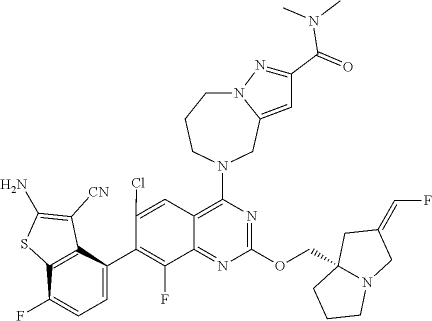

### 5-((R)-7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

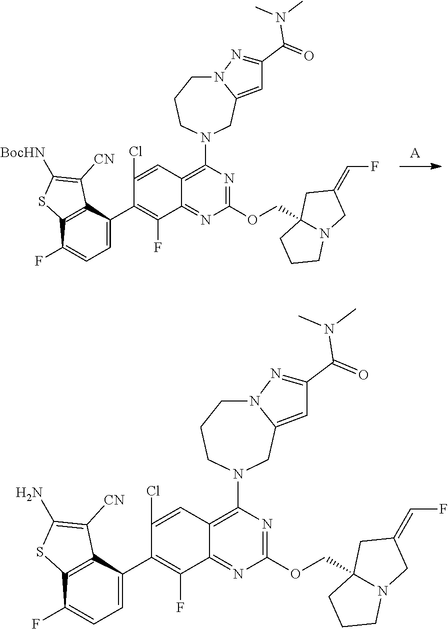

Step A. 5-((R)-7-(2-amino-3-cyano-7-fluorobenzo[b]thiophen-4-yl)-6-chloro-8-fluoro-2-(((S,Z)-2-(fluoromethylene)tetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide:

To a solution of tert-butyl (4-((R)-6-chloro-4-(2-(dimethylcarbamoyl)-7,8-dihydro-4H-pyrazolo[1,5-a][1,4]diazepin-5(6H)-yl)-8-fluoro-2-(((S,Z)-2-(fluoromethylene)tetrahydro-1H-pyrrolizin-7a(5H)-yl)methoxy)quinazolin-7-yl)-3-cyano-7-fluorobenzo[b]thiophen-2-yl)carbamate (250 mg, 1.0 equiv) in DCM (2 mL) was added TFA (3.07 g, 91 equiv). The reaction was stirred at 20° C. for 0.5 hours. The mixture was concentrated in vacuum and basified with saturated aqueous NaHCO₃(3 mL). The aqueous phase was extracted with ethyl acetate (3×3 mL). The combined organic layers were dried over Na₂SO₄, concentrated and purified with prep-HPLC [column: Waters Xbridge 150×25 mm×5 μm; A: water (NH₄HCO₃), B: ACN; B %: 46%-76% over 9 min] to afford the title compound (107 mg, 48% yield) as off-white solid; ¹H NMR (400 MHz, methanol-d₄) δ=8.00 (d, J=1.2 Hz, 1H), 7.21 (dd, J=5.2, 8.4 Hz, 1H), 7.03 (t, J=9.6 Hz, 1H), 6.77-6.51 (m, 2H), 5.08 (s, 2H), 4.54 (t, J=5.2 Hz, 2H), 4.31-4.18 (m, 4H), 3.88-3.80 (m, 1H), 3.50-3.42 (m, 1H), 3.33 (s, 3H), 3.18-3.12 (m, 1H), 3.08 (s, 3H), 2.75-2.65 (m, 2H), 2.47-2.33 (m, 3H), 2.15-2.06 (m, 1H), 2.01-1.79 (m, 3H); LCMS (ESI, M+1): m/z=748.5.

### Example 196

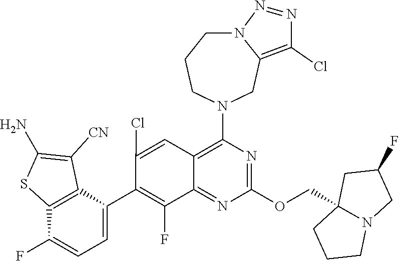

### 2-amino-4-((S)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

### Example 197

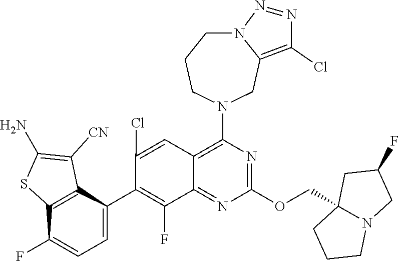

### 2-amino-4-((R)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

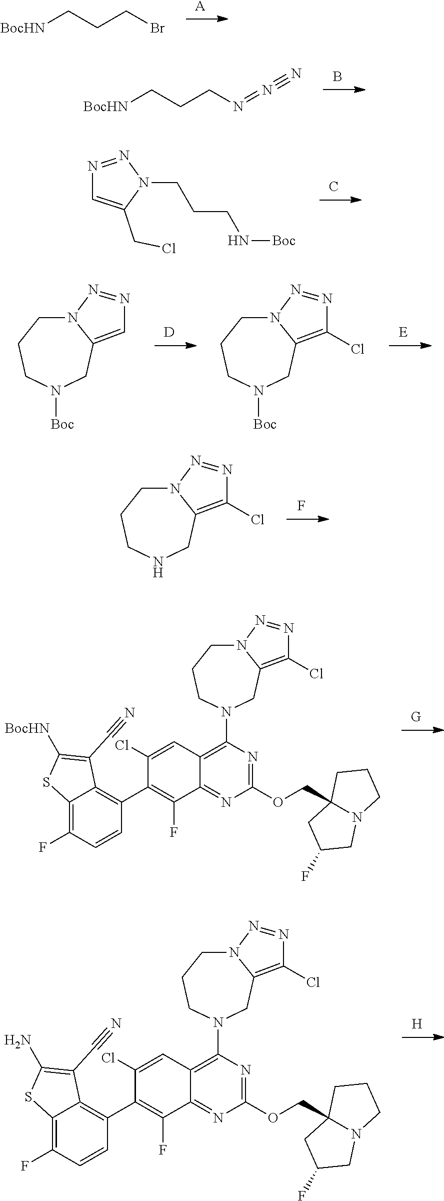

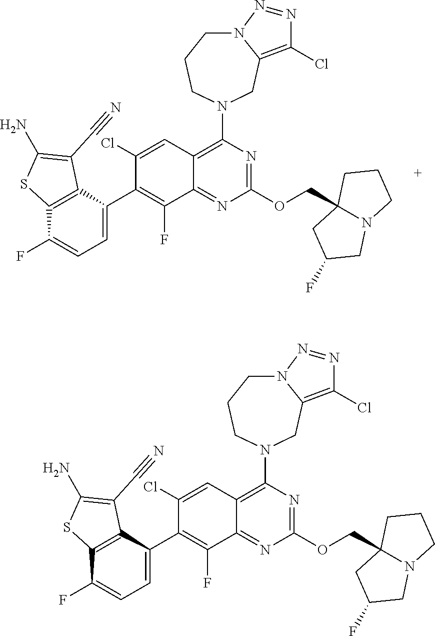

Step A. tert-butyl (3-azidopropyl)carbamate: To a solution of tert-butyl (3-bromopropyl)carbamate (20.0 g, 1.0 equiv) in DMF (200 mL) was added NaN₃ (6.95 g, 1.3 equiv). The reaction was stirred at 60° C. for 12 hours. The mixture was diluted with water (400 mL) and extracted with ethyl acetate (3×60 mL). The combined organic layers were dried over anhydrous sodium sulfate, concentrated to afford the title compound (20.0 g, crude) as colorless liquid.

Step B. tert-butyl (3-(5-(chloromethyl)-1H-1,2,3-triazol-1-yl)propyl)carbamate: To a mixture of tert-butyl (3-azidopropyl)carbamate (20 g, 1.0 equiv) in H₂O (100 mL) was added 3-chloroprop-1-yne (22.3 g, 3.0 equiv). The reaction was stirred at 80° C. for 48 hours. The mixture was extracted with ethyl acetate (3×40 mL). The combined organic layers were dried over anhydrous sodium sulfate, concentrated and purified by column chromatography [SiO₂, Petroleum ether/Ethyl acetate=20/1 to 1/1] to afford the title compound (7.50 g, 27% yield) as light yellow liquid; ¹H NMR (400 MHz, CHLOROFORM-d) δ=7.79-7.58 (m, 1H), 4.83 (br s, 1H), 4.74-4.63 (m, 2H), 4.51-4.33 (m, 2H), 3.24-3.06 (m, 2H), 2.23-2.05 (m, 2H), 1.44 (s, 9H); LCMS (ESI, M+1): m/z=275.0.

Step C. tert-butyl 7,8-dihydro-4H-[1,2,3]triazolo[1,5-a][1,4]diazepine-5(6H)-carboxylate: To a solution of tert-butyl (3-(5-(chloromethyl)-1H-1,2,3-triazol-1-yl)propyl)carbamate (1.5 g, 1.0 equiv) in THE (150 mL) was added NaH (437 mg, 60% purity, 2.0 equiv) at 0° C. The reaction was stirred at 60° C. for 36 hours. NaH (349 mg, 60% purity, 1.6 equiv) was added to above mixture at 0° C. The reaction was stirred at 60° C. for 24 hours. The mixture was diluted with water (40 mL) and extracted with ethyl acetate (3×30 mL). The combined organic layers were dried over anhydrous sodium sulfate, concentrated, and purified by column chromatography [Al₂O₃, Petroleum ether/Ethyl acetate=10/1 to 1/1] to afford the title compound (230 mg, 18% yield) as yellow liquid; LCMS (ESI, M+1): m/z=239.2.

Step D. tert-butyl 3-chloro-7,8-dihydro-4H-[1,2,3]triazolo[1,5-a][1,4]diazepine-5(6H)-carboxylate: To a solution of tert-butyl 7,8-dihydro-4H-[1,2,3]triazolo[1,5-a][1,4]diazepine-5(6H)-carboxylate (470 mg, 1.0 equiv) in MeCN (5 mL) was added NCS (342 mg, 1.3 equiv). The reaction was stirred at 60° C. for 1 hour. The mixture was concentrated. The residue was diluted with water (10 mL) and extracted with ethyl acetate (3×10 mL). The combined organic layers were dried over anhydrous sodium sulfate, concentrated, and purified by column chromatography [Al₂O₃, Petroleum ether/Ethyl acetate=10/1 to 10/1] to afford the title compound (410 mg, 73% yield) as light yellow liquid; LCMS (ESI, M+1): m/z=273.2.

Step E. 3-chloro-5,6,7,8-tetrahydro-4H-[1,2,3]triazolo[1,5-a][1,4]diazepine: To a solution of tert-butyl 3-chloro-7,8-dihydro-4H-[1,2,3]triazolo[1,5-a][1,4]diazepine-5(6H)-carboxylate (210 mg, 1.0 equiv) in MeOH (0.5 mL) was added HCl.MeOH (4 M, 2.10 mL, 11 equiv). The reaction was stirred at 0° C. for 0.5 hours. The mixture was concentrated. The residue was dissolved in MeOH. The mixture was adjusted to pH>7 with NaHCO₃, filtered and concentrated. The residue was dissolved in DCM/MeOH=10/1 and stirred for 10 mintues to afford the title compound (130 mg, crude) as yellow liquid; LCMS (ESI, M+1): m/z=172.9.

Step F. tert-butyl (4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (320 mg, 1.0 equiv) in DMSO (3 mL) were added TEA (207 μL, 3.0 equiv) and PyBOP (387 mg, 1.5 equiv). The reaction was stirred at 30° C. for 2 hours. 3-chloro-5,6,7,8-tetrahydro-4H-[1,2,3]triazolo[1,5-a][1,4]diazepine (128 mg, 1.5 equiv) was added. The reaction was stirred at 30° C. for 12 hours. The mixture was filtered and purified by reversed phase flash [C18, 0.1% formic acid condition] to afford the title compound (300 mg, 75% yield) as yellow solid; LCMS (ESI, M+1): m/z=800.2.

Step G. 2-amino-4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (440 mg, 1.0 equiv) in DCM (2 mL) was added TFA (4 mL, 98 equiv) at 0° C. The reaction was stirred at 25° C. for 0.5 hours. The mixture was adjusted pH≥8 with saturated NaHCO₃ aqueous solution (30 mL) and extracted with DCM (3×10 mL). The combined organic layers were dried over anhydrous sodium sulfate, concentrated and purified by reversed phase flash [C18, 0.1% formic acid condition] to afford the title compound (170 mg, 44% yield) as yellow solid; LCMS (ESI, M+1): m/z=700.1.

Step H. 2-amino-4-((S)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile and 2-amino-4-((R)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: The racemic product was purified with SFC [column: DAICEL CHIRALPAK AD (250 mm×30 mm, 10 m); A: CO₂-CAN, B: i-PrOH (0.1% NH₃.H₂O); B %: 50%, isocratic elution mode] to afford two peaks.

Peak 1: 2-amino-4-((S)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorohexahydro-1H-pyrrolizin-7a-yl)methoxy)quinazolin-7-yl)-7-fluorobenzo[b]thiophene-3-carbonitrile: The residue was purified with reversed phase flash [C18, 0.1% formic acid condition] to afford the title compound (50.4 mg, 29% yield) as yellow solid; ¹H NMR (400 MHz, METHANOL-d₄) δ=7.90 (d, J=1.2 Hz, 1H), 7.20 (dd, J=5.2, 8.4 Hz, 1H), 7.04 (t, J=8.8 Hz, 1H), 5.39-5.22 (m, 1H), 5.12-5.02 (m, 2H), 4.65 (br dd, J=3.2, 6.8 Hz, 2H), 4.41-4.29 (m, 2H), 4.21-4.10 (m, 2H), 3.38-3.34 (m, 1H), 3.25-3.09 (m, 3H), 3.04-2.95 (m, 1H), 2.47-2.21 (m, 3H), 2.20-2.05 (m, 2H), 2.05-1.81 (m, 3H); LCMS (ESI, M+1): m/z=700.2; SFC: 100% de, column: ChiralpakAD-3 50×4.6 mm I.D., 3 m Mobile phase: Phase A for CO₂, and Phase B for IPA+ACN (0.05% DEA); Gradient elution: 40% IPA+ACN (0.05% DEA) in CO₂ Flow rate: 3 mL/min; Detector: PDA; Column Temp: 35° C.; Back Pressure: 100 Bar, t_{R}: 0.609 min.

Peak 2: 2-amino-4-((R)-6-chloro-4-(3-chloro-7,8-dihydro-4H-[1,2,3]triazolo[1,5-a][1,4]diazepin-5(6H)-yl)-8-fluoro-2-(((2R,7aS)-2-fluorohexahydro-1H-pyrrolizin-7a-yl)methoxy)quinazolin-7-yl)-7-fluorobenzo[b]thiophene-3-carbonitrile: The residue was purified with reversed phase flash [C18, 0.1% formic acid condition] to afford the title compound (50.5 mg, 29% yield) as yellow solid; ¹H NMR (400 MHz, METHANOL-d₄) δ=7.90 (d, J=1.2 Hz, 1H), 7.20 (dd, J=5.2, 8.4 Hz, 1H), 7.04 (t, J=8.8 Hz, 1H), 5.39-5.22 (m, 1H), 5.10-5.03 (m, 2H), 4.68-4.63 (m, 2H), 4.42-4.27 (m, 2H), 4.22-4.09 (m, 2H), 3.34 (br d, J=3.6 Hz, 1H), 3.28-3.10 (m, 3H), 3.00 (dt, J=6.0, 9.2 Hz, 1H), 2.45-2.22 (m, 3H), 2.21-2.06 (m, 2H), 2.03-1.82 (m, 3H); LCMS (ESI, M+1): m/z=700.2; SFC: 99.4% de, column: Chiralpak AD-3 50×4.6 mm I.D., 3 m Mobile phase: Phase A for CO₂, and Phase B for IPA+ACN (0.05% DEA); Gradient elution: 40% IPA+ACN (0.05% DEA) in CO₂ Flow rate: 3 mL/min; Detector: PDA; Column Temp: 35° C.; Back Pressure: 100 Bar, t_{R}: 2.052 min.

### Example 198

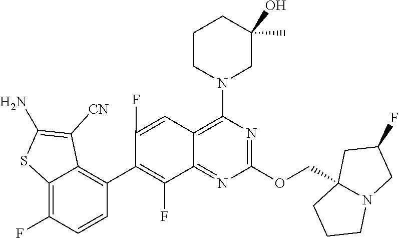 [SMILES: C[C@@]1(O)CCCN(c2nc(OC[C@@]34CCCN3C[C@H](F)C4)nc3c(F)c(-c4ccc(F)c5sc(N)c(C#N)c45)c(F)cc23)C1]

### 2-amino-4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile

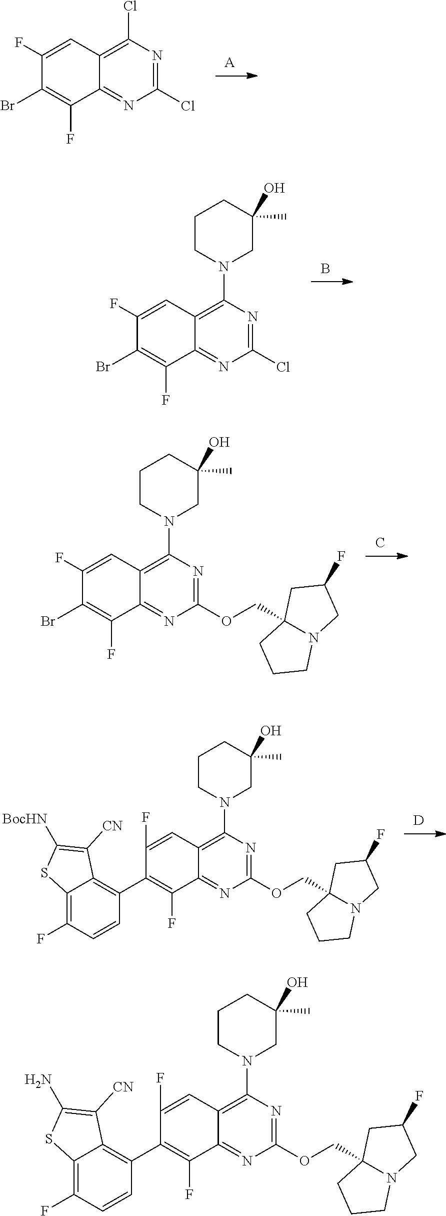

Step A. (R)-1-(7-bromo-2-chloro-6,8-difluoroquinazolin-4-yl)-3-methylpiperidin-3-01: To a solution of (R)-3-methylpiperidin-3-ol (241 mg, 1.0 equiv, HCl) in THF (10 mL) were added DIEA (1.03 g, 5.0 equiv) and 7-bromo-2,4-dichloro-6,8-difluoroquinazoline (500 mg, 1.0 equiv) at 0° C. The reaction was stirred at 0° C. for 1 hour. The mixture was diluted with water (20 mL) and extracted with ethyl acetate (20 mL×3). The combined organic layers were washed with brine (30 mL), dried over anhydrous sodium sulfate, concentrated and purified with column chromatography [SiO₂, petroleum ether/ethyl acetate=5/1 to 2/1] to afford the title compound (459 mg, 73% yield) as white solid; LCMS (ESI, M+1, M+3): m/z=392.1, 394.1.

Step B. (R)-1-(7-bromo-6,8-difluoro-2-(((2R,7aS)-2-fluorotetrahydro-1H-pyrrolizin-7a(5H)-yl)methoxy)quinazolin-4-yl)-3-methylpiperidin-3-ol: To a solution of (R)-1-(7-bromo-2-chloro-6,8-difluoroquinazolin-4-yl)-3-methylpiperidin-3-ol (459 mg, 1.0 equiv) and ((2R,7aS)-2-fluorotetrahydro-1H-pyrrolizin-7a(5H)-yl)methanol (223 mg, 1.2 equiv) in THE (5 mL) and DMF (5 mL) were added Cs₂CO₃ (1.14 g, 3.0 equiv) and DABCO (131 mg, 1.0 equiv). The reaction was degassed and purged with N₂ for 3 times and stirred at 25° C. for 12 hours under N₂ atmosphere. The mixture was diluted with water (20 mL) and extracted with ethyl acetate (15 mL×3). The combined organic layers were washed with brine (30 mL), dried over anhydrous sodium sulfate, concentrated and triturated with ethyl acetate (5 mL) to afford the title compound (275 mg, 42% yield) as white solid; LCMS (ESI, M+1, M+3): m/z=515.2, 517.2.

Step C. tert-butyl (3-cyano-4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophen-2-yl)carbamate: To a solution of (R)-1-(7-bromo-6,8-difluoro-2-(((2R,7aS)-2-fluorotetrahydro-1H-pyrrolizin-7a(5H)-yl)methoxy)quinazolin-4-yl)-3-methylpiperidin-3-ol (275 mg, 1.0 equiv) and tert-butyl (3-cyano-4-(5,5-dimethyl-1,3,2-dioxaborinan-2-yl)-7-fluorobenzo[b]thiophen-2-yl)carbamate (258 mg, 1.2 equiv) in toluene (5 mL) were added Cs₂CO₃ (347 mg, 2.0 equiv) and Pd(DPEphos)Cl₂ (36.7 mg, 0.1 equiv). The reaction was degassed and purged with N₂ for 3 times and stirred at 110° C. for 6 hours under N₂ atmosphere. The mixture was diluted with water (20 mL) and extracted with ethyl acetate (15 mL×3). The combined organic layers were washed with brine (30 mL), dried over anhydrous sodium sulfate, concentrated and purified with prep-HPLC [column: Phenomenex Luna C18 150×25 mm×10 um; mobile phase: [water (FA)−ACN]; gradient:26%-566% B over 10 min] to afford the title compound (150 mg, 37% yield) as brown solid; LCMS (ESI, M+1): m/z=727.4.

Step D. 2-amino-4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophene-3-carbonitrile: To a solution of tert-butyl (3-cyano-4-(6,8-difluoro-2-(((2R,7aS)-2-fluorotetrahydro-1H-pyrrolizin-7a(5H)-yl)methoxy)-4-((R)-3-hydroxy-3-methylpiperidin-1-yl)quinazolin-7-yl)-7-fluorobenzo[b]thiophen-2-yl)carbamate (90 mg, 1.0 equiv) in MeOH (1 mL) was added HCl.MeOH (1 mL, 4 M). The reaction was stirred at 25° C. for 12 hours. The mixture was concentrated, dissolved in MeOH (2 mL), adjusted to pH=7 with solid NaHCO₃, filtered and purified with prep-HPLC [column: Phenomenex luna C18 150×25 mm×10 um; mobile phase: [water (FA)−ACN]; gradient: 15%-45% B over 10 min] to afford the title compound (4.68 mg, 8.1% yield, 1CF₃COOH) as white solid; ¹H NMR (400 MHz, METHANOL-d4) δ=7.75 (dd, J=1.6, 10.0 Hz, 1H), 7.27 (dd, J=5.2, 8.4 Hz, 1H), 7.04 (dd, J=8.4, 9.2 Hz, 1H), 5.45-5.25 (m, 1H), 4.43-4.34 (m, 1H), 4.27 (d, J=10.8 Hz, 1H), 4.22 (br d, J=13.2 Hz, 1H), 4.04 (br d, J=13.2 Hz, 1H), 3.47 (d, J=13.2 Hz, 1H), 3.44-3.33 (m, 3H), 3.13-3.03 (m, 1H), 2.47-1.68 (m, 11H), 1.25 (s, 3H); LCMS (ESI, M+1): m/z=627.3.

### Example 199

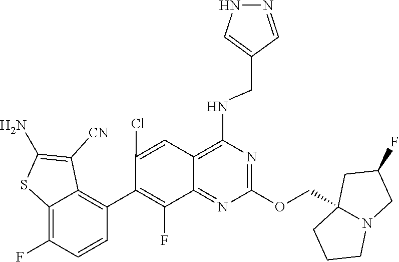 [SMILES: N#Cc1c(N)sc2c(F)ccc(-c3c(Cl)cc4c(NCc5cn[nH]c5)nc(OC[C@@]56CCCN5C[C@H](F)C6)nc4c3F)c12]

### 4-(4-(((1H-pyrazol-4-yl)methyl)amino)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

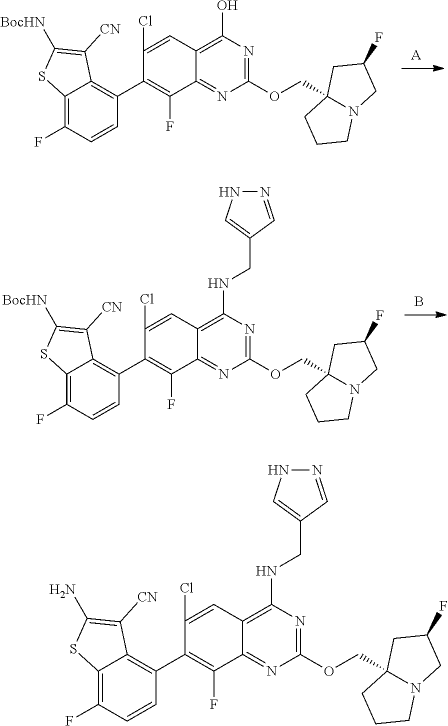

Step A. tert-butyl (4-(4-(((1H-pyrazol-4-yl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (90.0 mg, 1.0 equiv) in DMSO (1 mL) were added TEA (70.5 mg, 5 equiv) and PYBOP (109 mg, 1.5 equiv). The reaction was stirred at 20° C. for 0.5 hours. The mixture was added 1H-pyrazol-4-ylmethanamine (27.9 mg, 1.5 equiv, HCl) and stirred at 20° C. for 2.5 hours. The mixture was added water (2 mL) and extracted with ethyl acetate (3×3 mL). The combined organic layers were washed with saturated aqueous NaCl (3×5 mL), dried over Na₂SO₄ and concentrated to afford the title compound (90.0 mg, crude) as a yellow solid.

Step B. 4-(4-(((1H-pyrazol-4-yl)methyl)amino)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of tert-butyl (4-(4-(((1H-pyrazol-4-yl)methyl)amino)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (80 mg, 1.0 equiv) in DCM (0.5 mL) was added TFA (767 mg, 61 equiv). The reaction was stirred at 20° C. for 0.5 hours. The mixture was concentrated in vacuum and basified with saturated aqueous NaHCO₃(1.0 mL). The aqueous phase was extracted with ethyl acetate (3×1 mL). The combined organic layers were dried over Na₂SO₄ and concentrated in vacuum. The mixture was purified by prep-HPLC [column: Phenomenex luna C18 150×25 mm×10 μm; A: water (FA), B: ACN; B %: 20%-50% over 9 min]. The desired fractions were collected and concentrated under vacuum to remove ACN. The aqueous layers was lyophilized to afford the title compound (6.0 mg, 8.4% yield, 0.37 FA) as a white solid; LCMS (ESI, M+1): m/z=625.0; ¹H NMR (400 MHz, METHANOL-d₄) δ=8.13 (d, J=1.2 Hz, 1H), 7.69 (s, 2H), 7.20 (dd, J=5.2, 8.4 Hz, 1H), 7.08-7.00 (m, 1H), 5.51-5.31 (m, 1H), 4.81-4.70 (m, 2H), 4.56-4.38 (m, 2H), 3.67-3.45 (m, 3H), 3.25-3.15 (m, 1H), 2.56-2.34 (m, 2H), 2.34-2.22 (m, 1H), 2.19-2.08 (m, 2H), 2.05-1.92 (m, 1H).

### Example 200

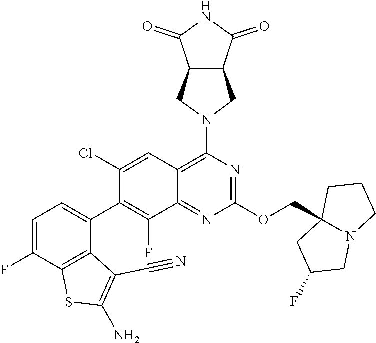 [SMILES: N#Cc1c(N)sc2c(F)ccc(-c3c(Cl)cc4c(N5C[C@@H]6C(=O)NC(=O)[C@@H]6C5)nc(OC[C@@]56CCCN5C[C@H](F)C6)nc4c3F)c12]

### 2-amino-4-(6-chloro-4-((3aR,6aS)-4,6-dioxohexahydropyrrolo[3,4-c]pyrrol-2(1H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

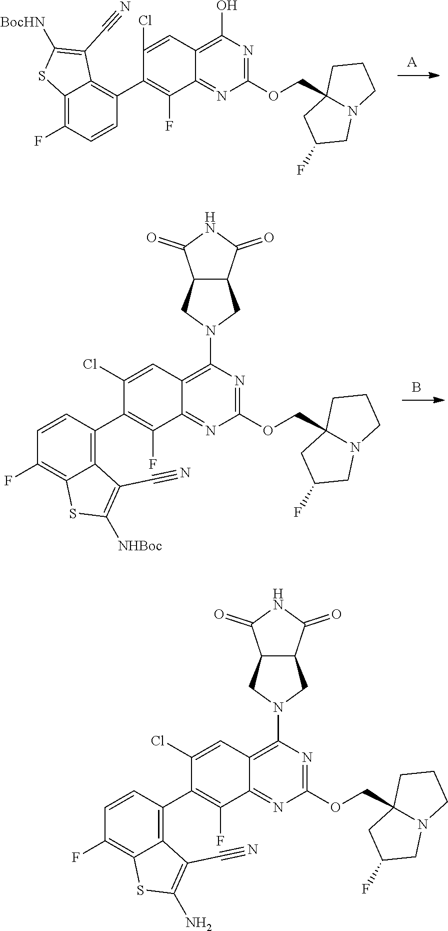

Step A. tert-butyl (4-(6-chloro-4-((3aR,6aS)-4,6-dioxohexahydropyrrolo[3,4-c]pyrrol-2(1H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 equiv) in DMSO (0.5 mL) were added TEA (39.2 mg, 5.0 equiv) and PYBOP (60.4 mg, 1.5 equiv). The reaction was stirred at 25° C. for 20 minutes and then (3aR,6aS)-tetrahydropyrrolo[3,4-c]pyrrole-1,3(2H,3aH)-dione (16.3 mg, 1.5 equiv) was added. The reaction was stirred at 25° C. for 1 hour. The mixture was diluted with water (10 mL) and extracted with ethyl acetate (10 mL×2). The combined organic layers were washed with brine (10 mL), dried over anhydrous sodium sulfate, concentrated and purified with prep-TLC (SiO₂, DCM/MeOH=10/1) to afford the title compound (45.0 mg, 62% yield) as yellow solid; LCMS (ESI, M+1): m/z=768.3.

Step B. 2-amino-4-(6-chloro-4-((3aR,6aS)-4,6-dioxohexahydropyrrolo[3,4-c]pyrrol-2(1H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-((3aR,6aS)-4,6-dioxohexahydropyrrolo[3,4-c]pyrrol-2(1H)-yl)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (40.0 mg, 1.0 equiv) in DCM (1 mL) was added TFA (0.5 mL). The mixture was stirred at 0° C. for 2 hours. The mixture was concentrated, dissolved in MeOH (2 mL), adjusted to pH=7 with solid NaHCO₃ and filtered. The filtrate was concentrated and purified with prep-HPLC (column: Phenomenex luna C18 150×25 mm×10 um; mobile phase: [water (FA)−ACN]; gradient: 20%-50% B over 7 min) to afford the title compound (7.99 mg, 23% yield, 0.47HCOOH) as white solid; ¹H NMR (400 MHz, METHANOL-d₄) δ=8.13 (s, 1H), 7.22 (dd, J=5.2, 8.4 Hz, 1H), 7.04 (t, J=9.6 Hz, 1H), 5.52-5.32 (m, 1H), 4.68-4.56 (m, 2H), 4.52-4.37 (m, 2H), 4.28-4.16 (m, 2H), 3.75-3.46 (m, 5H), 3.22 (dt, J=5.6, 10.0 Hz, 1H), 2.56-2.23 (m, 3H), 2.20-2.10 (m, 2H), 2.08-1.96 (m, 1H); LCMS (ESI, M+1): m/z=668.2.

### Example 201

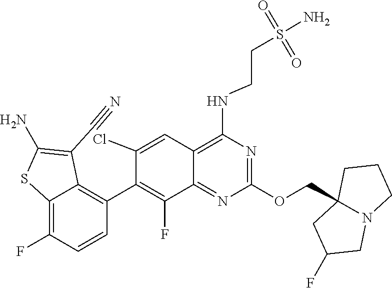 [SMILES: N#Cc1c(N)sc2c(F)ccc(-c3c(Cl)cc4c(NCCS(N)(=O)=O)nc(OC[C@@]56CCCN5CC(F)C6)nc4c3F)c12]

### 2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)ethane-1-sulfonamide

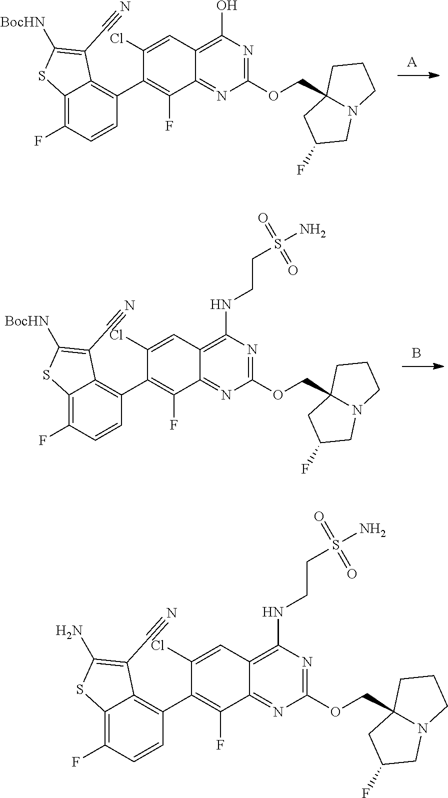

Step A. tert-butyl (4-(6-chloro-8-fluoro-2-(((2R,7aS)-2-fluorotetrahydro-1H-pyrrolizin-7a(5H)-yl)methoxy)-4-((2-sulfamoylethyl)amino)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 equiv) in DMSO (0.5 mL) were added TEA (39.2 mg, 5.0 equiv) and PyBOP (60.4 mg, 1.5 equiv). The reaction was stirred at 25° C. for 0.5 hours and then 2-aminoethane-1-sulfonamide (14.4 mg, 1.5 equiv) was added. The reaction was stirred at 25° C. for 1 hour. The mixture was diluted with water (10 mL) and extracted with ethyl acetate (10 mL×2). The combined organic layers were washed with brine (10 mL), dried over anhydrous sodium sulfate, concentrated and purified with prep-TLC [SiO₂, DCM/MeOH=10/1] to afford the title compound (50.0 mg, 82% yield) as yellow solid; LCMS (ESI, M+1): m/z=752.2.

Step B. 2-((7-(2-amino-3-cyano-7-fluorobenzo[b]thiophen-4-yl)-6-chloro-8-fluoro-2-(((2R,7aS)-2-fluorotetrahydro-1H-pyrrolizin-7a(5H)-yl)methoxy)quinazolin-4-yl)amino)ethane-1-sulfonamide: To a solution of tert-butyl (4-(6-chloro-8-fluoro-2-(((2R,7aS)-2-fluorotetrahydro-1H-pyrrolizin-7a(5H)-yl)methoxy)-4-((2-sulfamoylethyl)amino)quinazolin-7-yl)-3-cyano-7-fluorobenzo[b]thiophen-2-yl)carbamate (40.0 mg, 1.0 equiv) in MeCN (0.5 mL) was added HCl.dioxane (4M, 0.5 mL). The reaction was stirred at 25° C. for 0.5 hours. The mixture was concentrated, dissolved in MeOH (1 mL), adjusted to pH=7 with solid NaHCO₃ and filtered. The filtrate was concentrated and purified with prep-HPLC [column: Waters Xbridge 150×25 mm×5 μm; mobile phase: water (NH₄HCO₃)−ACN; gradient: 35%-65% B over 9 min] to afford the title compound (7.95 mg, 22% yield) as white solid; ¹H NMR (400 MHz, METHANOL-d4) 6=8.01 (d, J=1.6 Hz, 1H), 7.19 (dd, J=5.2, 8.4 Hz, 1H), 7.06-7.00 (m, 1H), 5.43-5.18 (m, 1H), 4.35-4.23 (m, 2H), 4.12-4.03 (m, 2H), 3.57-3.47 (m, 2H), 3.29-3.12 (m, 3H), 3.07-2.96 (m, 1H), 2.41-1.89 (m, 6H); LCMS (ESI, M+1): m/z=652.3.

### Example 202

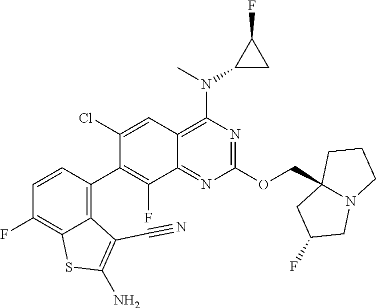 [SMILES: CN(c1nc(OC[C@@]23CCCN2C[C@H](F)C3)nc2c(F)c(-c3ccc(F)c4sc(N)c(C#N)c34)c(Cl)cc12)[C@H]1C[C@@H]1F]

### 2-amino-4-(6-chloro-8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)quinazolin-7-yl)-7-fluorobenzo[b]thiophene-3-carbonitrile

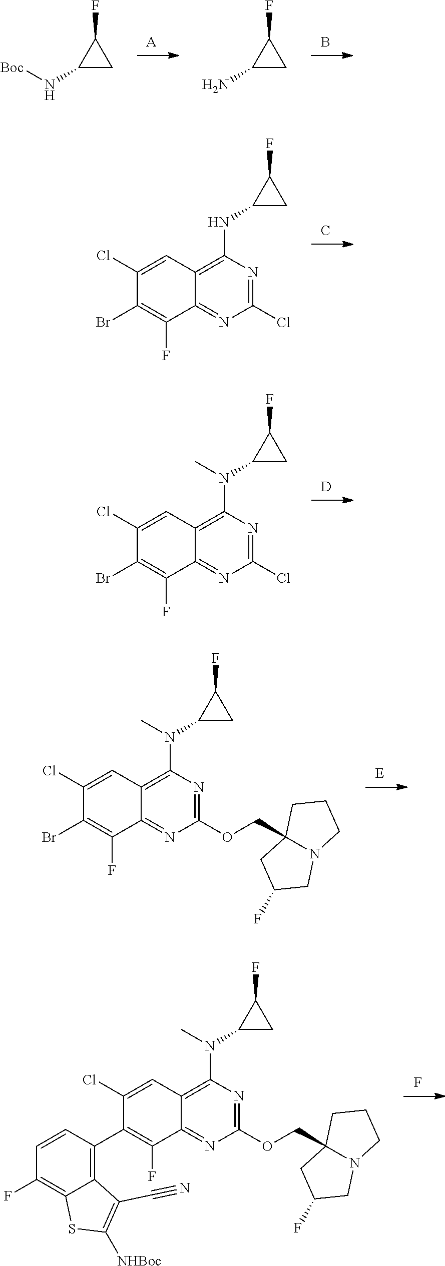

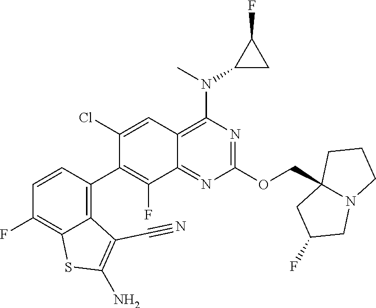 [SMILES: CN(c1nc(OC[C@@]23CCCN2C[C@H](F)C3)nc2c(F)c(-c3ccc(F)c4sc(N)c(C#N)c34)c(Cl)cc12)[C@H]1C[C@@H]1F]

Step A. (1S,2S)-2-fluorocyclopropan-1-amine: To a solution of tert-butyl ((1S,2S)-2-fluorocyclopropyl)carbamate (300 mg, 1.0 equiv) in DCM (1.5 mL) was added TFA (4.61 g, 23 equiv) at 0° C. The reaction was stirred at 20° C. for 0.5 hours. The mixture was concentrated to afford the title compound (345 mg, crude, TFA salt) as yellow liquid.

Step B. 7-bromo-2,6-dichloro-8-fluoro-N-((1S,2S)-2-fluorocyclopropyl)quinazolin-4-amine: To a solution of 7-bromo-2,4,6-trichloro-8-fluoroquinazoline (600 mg, 1.0 equiv) and DIEA (1.41 g, 6.0 equiv) in DCM (6 mL) was added (1S,2S)-2-fluorocyclopropan-1-amine (343 mg, 1.0 equiv, TFA salt) at −40° C. The reaction was stirred at −40° C. for 1 hour. The mixture was diluted with water (20 mL) and extracted with DCM (3×10 mL). The combined organic layers were dried over anhydrous sodium sulfate, concentrated and purified by reversed phase flash [C18, 0.1% formic acid condition] to afford the title compound (300 mg, 44% yield) as yellow solid; LCMS (ESI, M+1, M+3): m/z=367.9, 369.9.

Step C. 7-bromo-2,6-dichloro-8-fluoro-N-((1S,2S)-2-fluorocyclopropyl)-N-methylquinazolin-4-amine: To a solution of 7-bromo-2,6-dichloro-8-fluoro-N-((1S,2S)-2-fluorocyclopropyl)quinazolin-4-amine (300 mg, 1.0 equiv) and K₂CO₃ (337 mg, 3.0 equiv) in DMF (3 mL) was added MeI (2.31 g, 20 equiv). The reaction was stirred at 20° C. for 12 hours. The mixture was filtered and purified by reversed phase flash [C18, 0.1% formic acid condition] to afford the title compound (200 mg, 59% yield) as yellow solid; LCMS (ESI, M+1, M+3): m/z=381.9, 383.9.

Step D. 7-bromo-6-chloro-8-fluoro-N-((1S,2S)-2-fluorocyclopropyl)-2-(((2R,7aS)-2-fluorotetrahydro-1H-pyrrolizin-7a(5H)-yl)methoxy)-N-methylquinazolin-4-amine: To a solution of 7-bromo-2,6-dichloro-8-fluoro-N-((1S,2S)-2-fluorocyclopropyl)-N-methylquinazolin-4-amine (200 mg, 1.0 equiv) and ((2R,7aS)-2-fluorotetrahydro-1H-pyrrolizin-7a(5H)-yl)methanol (332 mg, 4.0 equiv) in dioxane (2 mL) was added DIEA (202 mg, 3.0 equiv). The reaction was stirred at 100° C. for 18 hours. The mixture was diluted with water (10 mL) and filtered to afford the title compound (110 mg, 38% yield) as yellow solid; LCMS (ESI, M+1, M+3): m/z=505.1, 507.1.

Step E. tert-butyl (4-(6-chloro-8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of 7-bromo-6-chloro-8-fluoro-N-((1S,2S)-2-fluorocyclopropyl)-2-(((2R,7aS)-2-fluorotetrahydro-1H-pyrrolizin-7a(5H)-yl)methoxy)-N-methylquinazolin-4-amine (50.0 mg, 1.0 equiv), tert-butyl (3-cyano-4-(5,5-dimethyl-1,3,2-dioxaborinan-2-yl)-7-fluorobenzo[b]thiophen-2-yl)carbamate (44.0 mg, 1.1 equiv) and Cs₂CO₃ (96.6 mg, 3.0 equiv) in THE (0.5 mL) was added dichloro[bis(2-(diphenylphosphino)phenyl)ether]palladium(II) (13.6 mg, 0.2 equiv). The reaction was degassed and purged with nitrogen 3 times. The reaction was stirred at 60° C. for 3 hours. The mixture was diluted with water (10 mL) and extracted with ethyl acetate (3×5 mL). The combined organic layers were dried over anhydrous sodium sulfate, concentrated, and purified by reversed phase flash [C18, 0.1% formic acid condition] to afford the title compound (30.0 mg, 41% yield) as yellow solid; LCMS (ESI, M+1): m/z=717.3.

Step F. 2-amino-4-(6-chloro-8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-8-fluoro-4-(((1S,2S)-2-fluorocyclopropyl)(methyl)amino)-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30.0 mg, 1.0 equiv) in DCM (0.25 mL) was added TFA (767 mg, 160 equiv). The reaction was stirred at 25° C. for 0.5 hours. The mixture was adjusted pH to 8 with saturated NaHCO₃ aqueous solution (10 mL) and extracted with DCM (3×5 mL). The combined organic layers were dried over anhydrous sodium sulfate, concentrated and purified with prep-HPLC [Phenomenex luna C18 150×25 mm×10 μm; A: water (FA), B: ACN; B %: 19%-49% over 10 min] to afford the title compound (12.9 mg, 50% yield, 0.36 HCOOH) as white solid; ¹H NMR (400 MHz, METHANOL-d₄) δ=8.42 (d, J=1.6 Hz, 1H), 7.23 (dd, J=5.0, 8.4 Hz, 1H), 7.05 (dd, J=8.4, 9.6 Hz, 1H), 5.47-5.32 (m, 1H), 4.65-4.56 (m, 1H), 4.54-4.31 (m, 2H), 3.91-3.79 (m, 1H), 3.64-3.45 (m, 3H), 3.37 (d, J=4.8 Hz, 3H), 3.19 (dt, J=5.4, 10.0 Hz, 1H), 2.54-2.39 (m, 1H), 2.39-2.07 (m, 4H), 2.04-1.90 (m, 1H), 1.71-1.53 (m, 1H), 1.31-1.11 (m, 1H); LCMS (ESI, M+1): m/z=617.2.

### Example 203

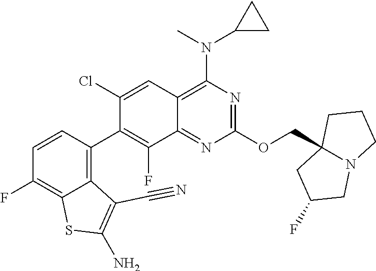 [SMILES: CN(c1nc(OC[C@@]23CCCN2C[C@H](F)C3)nc2c(F)c(-c3ccc(F)c4sc(N)c(C#N)c34)c(Cl)cc12)C1CC1]

### 2-amino-4-(6-chloro-4-(cyclopropyl(methyl)amino)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

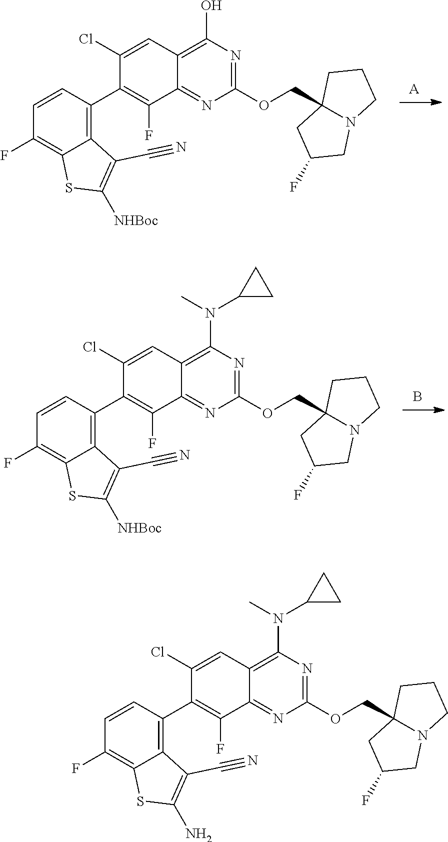

Step A. tert-butyl (4-(6-chloro-4-(cyclopropyl(methyl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (80.0 mg, 1.0 equiv), PyBOP (96.7 mg, 1.5 equiv) and TEA (37.6 mg, 3.0 equiv) in DMSO (1.0 mL) was added N-methylcyclopropanamine (17.6 mg, 2.0 equiv). The reaction was stirred at 30° C. for 12 hours. The mixture was diluted with water (5 mL) and extracted with EtOAc (3×5 mL). The combined organic layers were washed with brine (10 mL), dried over anhydrous sodium sulfate, concentrated and purified with reversed phase flash chromatography [C18, 0.1% formic acid condition] to afford the title compound (70 mg, 78% yield) as yellow solid; LCMS (ESI, M+1): m/z=699.3.

Step B. 2-amino-4-(6-chloro-4-(cyclopropyl(methyl)amino)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-(cyclopropyl(methyl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (50.0 mg, 1.0 equiv) in DCM (0.5 mL) was added TFA (1.0 mL). The reaction was stirred at 0° C. for 0.5 hours. The residue was adjusted to PH>8 with saturated NaHCO₃ aqueous (5 mL) at 0° C. and extracted with DCM (3×5 mL). The combined organic layers were washed with brine (10 mL), dried over anhydrous sodium sulfate, concentrated and purified with prep-HPLC [Phenomenex luna C18 150×25 mm×10 μm; A: water (FA), B: ACN; B %: 20%-50% over 10 min] and lyophilized to afford the title compound (6.84 mg, 16% yield, 0.21HCOOH) as white solid; ¹H NMR (400 MHz, DMSO-d₆) δ=8.46 (s, 1H), 8.09 (s, 2H), 7.30-7.27 (m, 1H), 7.14 (t, J=8.8 Hz, 1H), 5.40-5.17 (m, 1H), 4.17-3.92 (m, 2H), 3.63-3.55 (m, 1H), 3.27 (s, 3H), 3.17-3.03 (m, 3H), 2.85 (br d, J=7.2 Hz, 1H), 2.18-1.98 (m, 3H), 1.92-1.72 (m, 3H), 0.99-0.86 (m, 2H), 0.77-0.57 (m, 2H); LCMS (ESI, M+1): m/z=599.2.

### Example 204

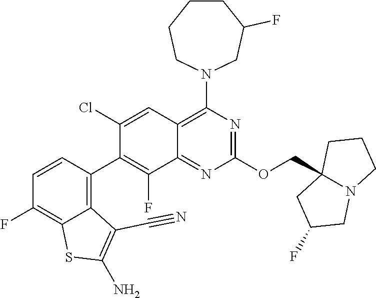 [SMILES: N#Cc1c(N)sc2c(F)ccc(-c3c(Cl)cc4c(N5CCCCC(F)C5)nc(OC[C@@]56CCCN5C[C@H](F)C6)nc4c3F)c12]

### 2-amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile

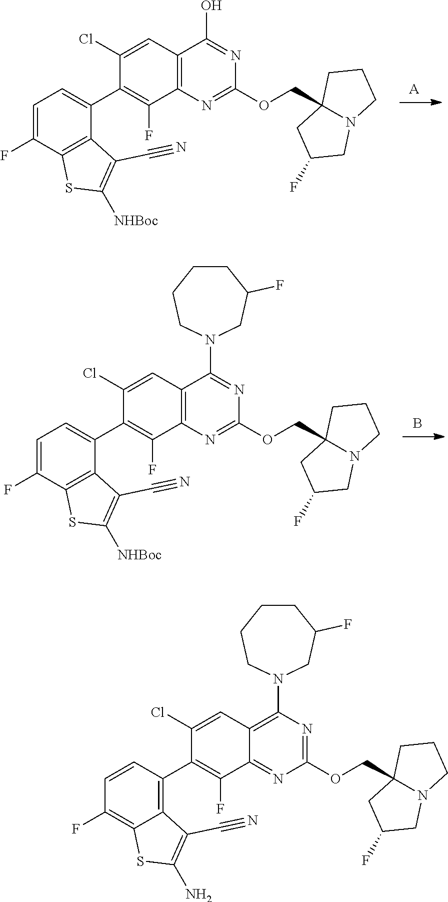

Step A. tert-butyl (4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (80.0 mg, 1.0 equiv) in DMSO (1 mL) was added TEA (51.7 μL, 3.0 equiv) and PyBOP (96.7 mg, 1.5 equiv). The reaction was stirred at 30° C. for 1 hour. 3-fluoroazepane (29.0 mg, 2.0 equiv) was added to the mixture. The reaction was stirred at 30° C. for 12 hours. The mixture was filtered and purified by reversed phase flash [C18, 0.1% formic acid condition] and lyophilized to afford the title compound. (60.0 mg, 64% yield) as yellow solid; LCMS (ESI, M+1): m/z=745.2.

Step B. 2-amino-4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-8-fluoro-4-(3-fluoroazepan-1-yl)-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (55.0 mg, 1.0 equiv) in DCM (0.25 mL) was added TFA (0.5 mL, 91 equiv) at 0° C. The reaction was stirred at 20° C. for 0.5 hours. The mixture was adjusted pH to 8 with saturated NaHCO₃ aqueous solution (10 mL) and extracted with DCM (3×5 mL). The combined organic layers were dried over anhydrous sodium sulfate, concentrated and purified with prep-HPLC [Waters xbridge 150×25 mm×10 μm; A: water (NH₄HCO₃), B: ACN; B %: 55%-85% over 10 min] and prep-HPLC [Phenomenex luna C18 150×25 mm×10 μm; A: water (FA), B: ACN; B %: 26%-46% over 10 min] and lyophilized to afford the title compound (18.3 mg, 38% yield, 0.22 HCOOH) as yellow solid; ¹H NMR (400 MHz, METHANOL-d₄) δ=8.12 (d, J=8.4 Hz, 1H), 7.21 (dd, J=5.2, 8.4 Hz, 1H), 7.11-6.95 (m, 1H), 5.49-5.29 (m, 1H), 5.19-5.02 (m, 1H), 4.49-4.28 (m, 3H), 4.28-4.08 (m, 2H), 4.08-3.92 (m, 1H), 3.62-3.49 (m, 1H), 3.48-3.38 (m, 2H), 3.25-3.08 (m, 1H), 2.52-2.37 (m, 1H), 2.37-2.27 (m, 1H), 2.26-2.18 (m, 1H), 2.18-1.85 (m, 8H), 1.75-1.47 (m, 1H); LCMS (ESI, M+1): m/z=645.3.

### Example 205

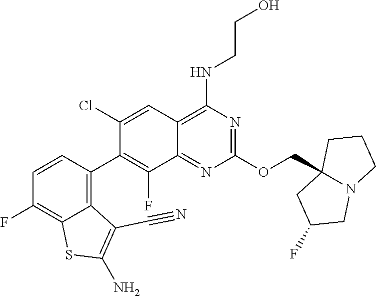 [SMILES: N#Cc1c(N)sc2c(F)ccc(-c3c(Cl)cc4c(NCCO)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-((2-hydroxyethyl)amino)quinazolin-7-yl)-7-fluorobenzo[b]thiophene-3-carbonitrile

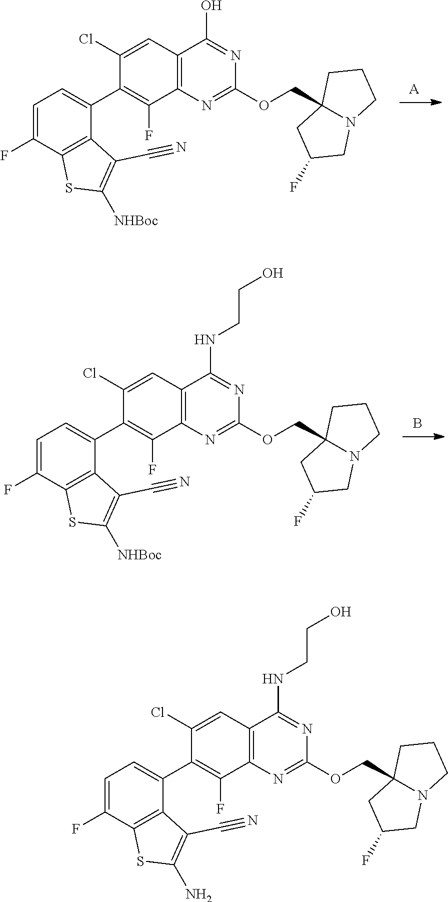

Step A. tert-butyl (4-(6-chloro-8-fluoro-2-(((2R,7aS)-2-fluorohexahydro-1H-pyrrolizin-7a-yl)methoxy)-4-((2-hydroxyethyl)amino)quinazolin-7-yl)-3-cyano-7-fluorobenzo[b]thiophen-2-yl)carbamate: To a solution of tert-butyl (4-(6-chloro-8-fluoro-2-(((2R, 7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (90.0 mg, 1.0 equiv) in DMSO (0.9 mL) were added TEA (84.6 mg, 6.0 equiv) and PyBOP (181 mg, 2.5 equiv). The reaction was stirred at 20° C. for 0.5 hours. 2-aminoethan-1-ol (51.0 mg, 6.0 equiv) was added into the mixture. The reaction was stirred at 20° C. for 24 hours. The mixture was filtered and purified by reversed phase flash [water (0.1% formic acid)/acetonitrile=1/1] to afford the title compound (100 mg, 96% yield) as yellow gum; LCMS (ESI, M+1): m/z=689.1.

Step B. 2-amino-4-(6-chloro-8-fluoro-2-(((2R,7aS)-2-fluorohexahydro-1H-pyrrolizin-7a-yl)methoxy)-4-((2-hydroxyethyl)amino)quinazolin-7-yl)-7-fluorobenzo[b]thiophene-3-carbonitrile: To a solution of tert-butyl (4-(6-chloro-8-fluoro-2-(((2R,7aS)-2-fluorohexahydro-1H-pyrrolizin-7a-yl)methoxy)-4-((2-hydroxyethyl)amino)quinazolin-7-yl)-3-cyano-7-fluorobenzo[b]thiophen-2-yl)carbamate (95.0 mg, 1.0 equiv) in DCM (1.0 mL) was added TFA (2.30 g, 146 equiv) at 0° C. The reaction was stirred at 20° C. for 1 hour. The mixture was dropped in the saturated NaHCO₃ aqueous solution (20 mL) and DCM (20 mL) at 0° C. The mixture was extracted with DCM (4×10 mL). The combined organic layers were washed with brine (30 mL), dried over anhydrous sodium sulfate, concentrated and purified with prep-HPLC [column: Waters Xbridge 150×25 mm×5 μm; mobile phase: [water (ammonia hydroxide v/v)−acetonitrile]; gradient: 35%-65% B over 10 minutes] twice to afford the tittle compound (21.9 mg, 25% yield, 0.52 HCCOH) as white solid; ¹H NMR (400 MHz, DMSO-d6) δ=8.63-8.56 (m, 1H), 8.32 (s, 1H), 8.16 (s, 1H), 8.10 (s, 2H), 7.26-7.19 (m, 1H), 7.18-7.10 (m, 1H), 5.37-5.18 (m, 1H), 5.06-4.62 (m, 1H), 4.11-4.05 (m, 1H), 3.99 (dd, J=2.4, 10.4 Hz, 1H), 3.68-3.60 (m, 4H), 3.13-3.06 (m, 2H), 3.02 (s, 1H), 2.87-2.79 (m, 1H), 2.20-1.95 (m, 3H), 1.89-1.70 (m, 3H); LCMS (ESI, M+1): m/z=589.1.

### Example 206

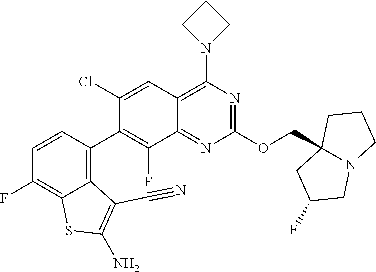 [SMILES: N#Cc1c(N)sc2c(F)ccc(-c3c(Cl)cc4c(N5CCC5)nc(OC[C@@]56CCCN5C[C@H](F)C6)nc4c3F)c12]

### 2-amino-4-(4-(azetidin-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

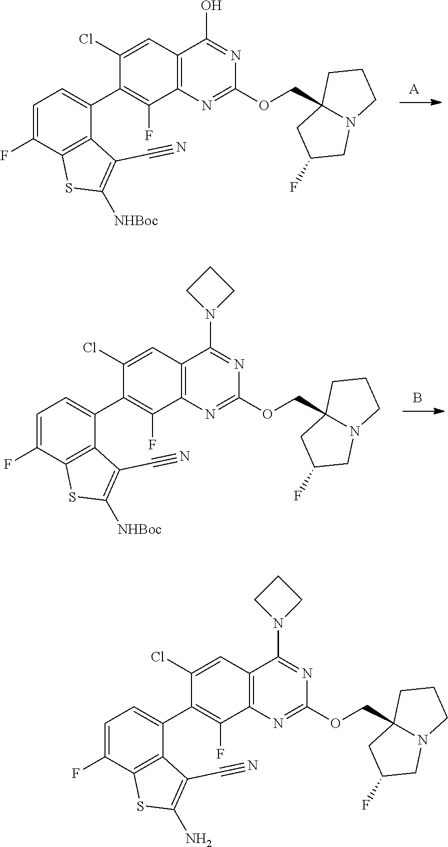

Step A. tert-butyl (4-(4-(azetidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) in DMSO (1 mL) were added PyBOP (242 mg, 3.0 equiv) and TEA (47.0 mg, 3.0 equiv). The reaction was stirred at 25° C. for 1 hour. To the mixture was added azetidine (26.5 mg, 3.0 equiv). The reaction was stirred at 25° C. for 12 hours. The mixture was diluted with water (10 mL) and extracted with ethyl acetate (2×10 mL). The combined organic layers were washed with brine (10 mL), dried over anhydrous sodium sulfate, concentrated and purified with reversed phase flash [C18, 0.1% formic acid condition] to afford the title compound (50.0 mg, 47% yield) as yellow solid; LCMS (ESI, M+1): m/z=685.2.

Step B. 2-amino-4-(4-(azetidin-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(4-(azetidin-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (40.0 mg, 1.0 equiv) in DCM (0.3 mL) was added TFA (0.8 mL) at 0° C. The reaction was stirred at 0° C. for 1 hour. The mixture was adjusted to pH=7 with saturated NaHCO₃(10 mL) and extracted with DCM (2×10 mL). The combined organic layers were dried over anhydrous sodium sulfate, concentrated and purified by prep-HPLC [column: Waters Xbridge 150×25 mm×5 μm; A: water (ammonia hydroxide v/v), B: ACN, B %: 48%-78% over 10 min] to afford the title compound (25.1 mg, 73% yield) as white solid; ¹H NMR (400 MHz, METHANOL-d4) δ=7.80 (s, 1H), 7.18 (dd, J=5.2, 8.4 Hz, 1H), 7.03 (t, J=8.8 Hz, 1H), 5.37 (br s, 1H), 4.33-4.25 (m, 1H), 4.23-4.16 (m, 1H), 3.36 (br d, J=1.6 Hz, 2H), 3.30-3.18 (m, 5H), 3.02 (dt, J=6.0, 9.2 Hz, 1H), 2.57 (quin, J=7.6 Hz, 2H), 2.41-2.20 (m, 2H), 2.17-2.11 (m, 1H), 2.03-1.83 (m, 3H); LCMS (ESI, M+1): m/z=585.2.

### Example 207

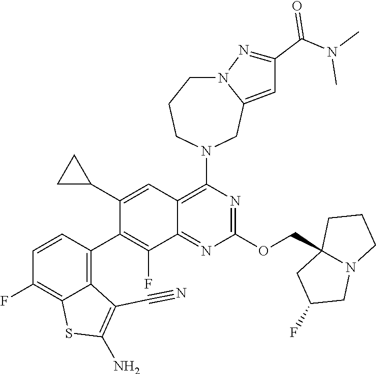 [SMILES: CN(C)C(=O)c1cc2n(n1)CCCN(c1nc(OC[C@@]34CCCN3C[C@H](F)C4)nc3c(F)c(-c4ccc(F)c5sc(N)c(C#N)c45)c(C4CC4)cc13)C2]

### 5-(7-(2-amino-3-cyano-7-fluorobenzo[b]thiophen-4-yl)-6-cyclopropyl-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

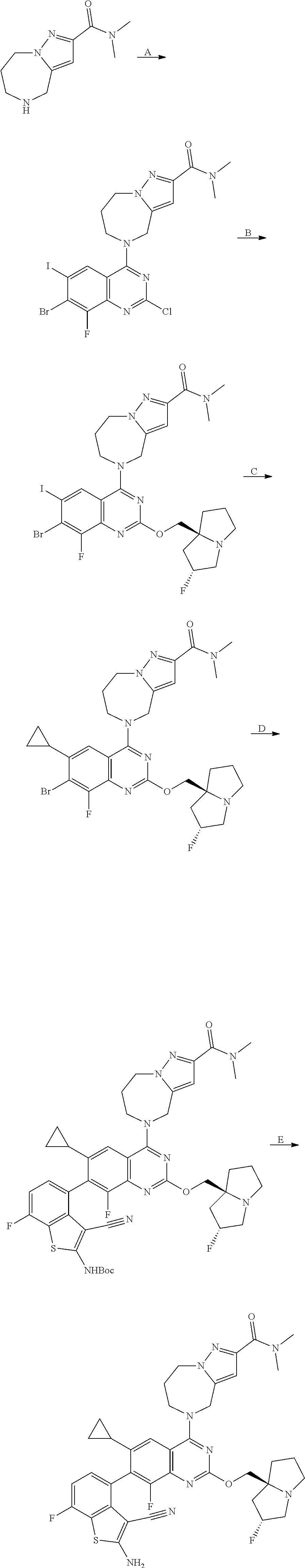

Step A. 5-(7-bromo-2-chloro-8-fluoro-6-iodoquinazolin-4-yl)-N,N-dimethyl-5,6,7,8-tetrahydro-4H-pyrazolo[1,5-a][1,4]diazepine-2-carboxamide. To a solution of 7-bromo-2,4-dichloro-8-fluoro-6-iodo-quinazoline (600 mg, 1.0 equiv) in DCM (10 mL) was added DIEA (919 mg, 1.24 mL, 5.0 equiv) and N,N-dimethyl-5,6,7,8-tetrahydro-4H-pyrazolo[1,5-a][1,4]diazepine-2-carboxamide (418 mg, 1.2 equiv, HCl). The reaction was stirred at 20° C. for 0.5 hours. The mixture was added water (5 mL) and extracted with ethyl acetate (3×5 mL). The combined organic layers were dried over Na₂SO₄ and concentrated to afford the title compound (900 mg, crude) as a yellow solid.

Step B. 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide. To a solution of 5-(7-bromo-2-chloro-8-fluoro-6-iodoquinazolin-4-yl)-N,N-dimethyl-5,6,7,8-tetrahydro-4H-pyrazolo[1,5-a][1,4]diazepine-2-carboxamide (800 mg, 1.0 equiv) in DMF (8 mL) and THF (8 mL) was added DABCO (151 mg, 1.0 equiv), ((2R,7aS)-2-fluorotetrahydro-1H-pyrrolizin-7a(5H)-yl)methanol (257 mg, 1.2 equiv) and Cs₂CO₃ (1.32 g, 3.0 equiv). The reaction was stirred at 20° C. for 2 hours. The mixture was diluted with water (10 mL) and extracted with ethyl acetate (3×10 mL). The combined organic layers were dried over Na₂SO₄, concentrated and purified by reversed phase flash chromatography [C18, 0.1% formic acid condition] to afford the title compound (400 mg, 40% yield) as a yellow solid; LCMS (ESI, M+1, M+3): m/z=716.1, 718.1.

Step C. 5-(7-bromo-6-cyclopropyl-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: A mixture of 5-(7-bromo-8-fluoro-2-(((2R,7aS)-2-fluorotetrahydro-1H-pyrrolizin-7a(5H)-yl)methoxy)-6-iodoquinazolin-4-yl)-N,N-dimethyl-5,6,7,8-tetrahydro-4H-pyrazolo[1,5-a][1,4]diazepine-2-carboxamide (200 mg, 1.0 equiv), cyclopropylboronic acid (28.8 mg, 1.2 equiv), Pd(dppf)Cl₂ (20.4 mg, 0.1 equiv) and K₃PO₄ (1.5 M, 3.0 equiv) in dioxane (5 mL) was degassed and purged with N₂ for 3 times, the reaction was stirred at 90° C. for 5 hours under N₂ atmosphere. The mixture was diluted with water (5 mL) and extracted with ethyl acetate (3×6 mL). The combined the organic layers were dried over anhydrous Na₂SO₄, concentrated and purified with silica gel chromatography (petroleum ether/ethyl acetate=30/1 to 0/1) to afford the title compound (130 mg, 70% yield) as a yellow solid; LCMS (ESI, M+1, M+3): m/z=630.2, 632.2.

Step D. tert-butyl (3-cyano-4-(6-cyclopropyl-4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate: A mixture of 5-(7-bromo-6-cyclopropyl-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide (60.0 mg, 1.0 equiv), tert-butyl N-[3-cyano-4-(5,5-dimethyl-1,3,2-dioxaborinan-2-yl)-7-fluoro-benzothiophen-2-yl]carbamate (42.3 mg, 1.1 equiv), Cs₂CO₃ (93.0 mg, 3.0 equiv) and dichloro[bis(2-(diphenylphosphino)phenyl)ether]palladium(II) (6.54 mg, 0.10 equiv) in THE (2 mL) was degassed and purged with N₂ for 3 times, the reaction was stirred at 60° C. for 2 hours under N₂ atmosphere. The mixture was added water (3 mL) and extracted with ethyl acetate (3×3 mL). The combined organic layers were dried over Na₂SO₄, concentrated and purified with reversed phase flash chromatography [C18, 0.1% formic acid condition] to afford the title compound (25.0 mg, 30% yield) as a yellow solid; LCMS (ESI, M+1): m/z=842.4.

Step E. 5-(7-(2-amino-3-cyano-7-fluorobenzo[b]thiophen-4-yl)-6-cyclopropyl-8-fluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: To a solution of tert-butyl (3-cyano-4-(6-cyclopropyl-4-(2-(dimethylcarbamoyl)-7,8-dihydro-4H-pyrazolo[1,5-a][1,4]diazepin-5(6H)-yl)-8-fluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate (20.0 mg, 1.0 equiv) in DCM (0.3 mL) was added TFA (460 mg, 170 equiv). The reaction was stirred at 20° C. for 0.5 hours. The mixture was concentrated in vacuum and basified with saturated aqueous NaHCO₃(1 mL). The aqueous phase was extracted with ethyl acetate (2×2 mL). The combined organic layers were dried over Na₂SO₄, concentrated and purified with prep-HPLC (column: C18 150×30 mm; A: water (FA), B: ACN; B %: 25%-55% over 7 min) to afford the title compound (12.2 mg, 68% yield, 0.16 FA) as a white solid; ¹H NMR (400 MHz, methanol-d₄) δ=7.40 (s, 1H), 7.22 (dd, J=4.8, 8.0 Hz, 1H), 7.04 (dd, J=8.4, 9.2 Hz, 1H), 6.72 (s, 1H), 5.46-5.27 (m, 1H), 5.08 (s, 2H), 4.54-4.48 (m, 2H), 4.38-4.23 (m, 4H), 3.56-3.37 (m, 3H), 3.36 (s, 3H), 3.17-3.11 (m, 1H), 3.09 (s, 3H), 2.47-2.26 (m, 4H), 2.25-2.16 (m, 1H), 2.12-2.02 (m, 2H), 2.00-1.88 (m, 1H), 1.67-1.58 (m, 1H), 0.81-0.63 (m, 4H); LCMS (ESI, M+1): m/z=742.3.

### Example 208

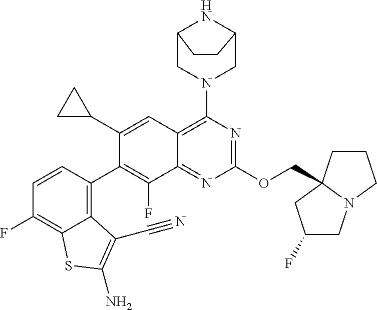 [SMILES: N#Cc1c(N)sc2c(F)ccc(-c3c(C4CC4)cc4c(N5CC6CCC(C5)N6)nc(OC[C@@]56CCCN5C[C@H](F)C6)nc4c3F)c12]

### 4-(4-((1R,5S)-3,8-diazabicyclo[3.2.1]octan-3-yl)-6-cyclopropyl-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

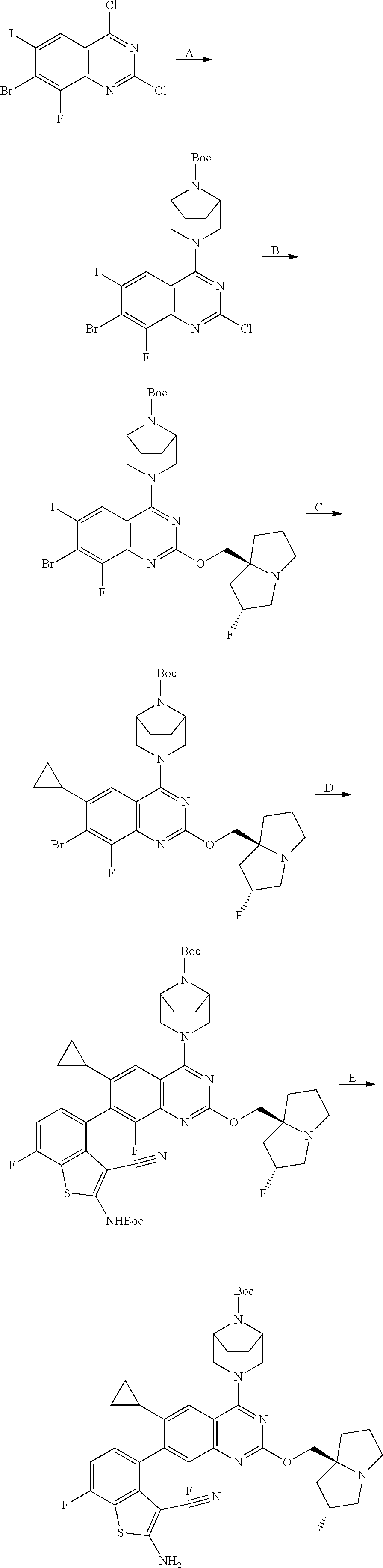

Step A. tert-butyl (1R,5S)-3-(7-bromo-2-chloro-8-fluoro-6-iodoquinazolin-4-yl)-3,8-diazabicyclo[3.2.1]octane-8-carboxylate: To a solution of 7-bromo-2,4-dichloro-8-fluoro-6-iodo-quinazoline (500 mg, 1.0 equiv) and TEA (360 mg 3.0 equiv) in DCM (20 mL) was added tert-butyl (1R,5S)-3,8-diazabicyclo[3.2.1]octane-8-carboxylate (252 mg, 1.0 equiv) at 0° C. The reaction was stirred at 0° C. for 1 hour. The mixture was diluted with water (10 mL) and extracted with dichloromethane (2×5 mL). The combined organic layers were dried over anhydrous sodium sulfate, concentrated to afford the title compound (800 mg, crude) as yellow solid; LCMS (ESI, M+1, M+3): m/z=597.1, 599.1.

Step B. tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate: To a solution of tert-butyl 3-(7-bromo-2-chloro-8-fluoro-6-iodo-quinazolin-4-yl)-3,8-diazabicyclo[3.2.1]octane-8-carboxylate (500 mg, 1.0 equiv), ((2R,7aS)-2-fluorotetrahydro-1H-pyrrolizin-7a(5H)-yl)methanol (399 mg, 3.0 equiv) and DABCO (93.8 mg, 1.0 equiv) in DMF (5 mL) and THF (5 mL) was added Cs₂CO₃ (818 mg, 3.0 equiv). The reaction was stirred at 25° C. for 2 hours. The mixture was diluted with water (50 mL) and extracted with ethyl acetate (2×50 mL). The combined organic layers were dried over anhydrous sodium sulfate, concentrated and purified with silica gel chromatography (SiO₂, Petroleum ether/Ethyl acetate=20/1 to 10/1) to afford the title compound (30 mg, 47% yield) as yellow solid; LCMS (ESI, M+1 M+3): m/z=720.2, 722.2.

Step C. tert-butyl (1R,5S)-3-(7-bromo-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-bromo-8-fluoro-2-(((2R,7aS)-2-fluorotetrahydro-1H-pyrrolizin-7a(5H)-yl)methoxy)-6-iodoquinazolin-4-yl)-3,8-diazabicyclo[3.2.1]octane-8-carboxylate (200 mg, 1.0 equiv), cyclopropylboronic acid (28.6 mg, 1.2 equiv) amd K₃PO₄ (1.5 M, 177 mg, 3.0 equiv) in dioxane (2 mL) was added Pd(dppf)Cl₂ (20.3 mg, 0.1 equiv) at 25° C. The reaction was stirred at 90° C. for 1 hour under N₂ atmosphere. The mixture was diluted with water (10 mL) and extracted with ethyl acetate (2×5 mL). The organic layers were dried over Na₂SO₄, concentrated and purified with reversed phase flash chromatography [C18, 0.1% NH₃.H₂O condition] to afford the title compound (170 mg, 87% yield) as yellow solid; LCMS (ESI, M+1, M+3): m/z=634.2, 636.2.

Step D. tert-butyl (1R,5S)-3-(7-(2-((tert-butoxycarbonyl)amino)-3-cyano-7-fluorobenzo[b]thiophen-4-yl)-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate: To a solution of tert-butyl (1R,5S)-3-(7-bromo-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (70.0 mg, 1.0 equiv), tert-butyl (3-cyano-4-(5,5-dimethyl-1,3,2-dioxaborinan-2-yl)-7-fluorobenzo[b]thiophen-2-yl)carbamate (57.9 mg, 1.3 equiv) and DPEPhosPdCl₂ (15.2 mg, 0.2 equiv) in dioxane (1 mL) was added Cs₂CO₃ (108 mg, 3.0 equiv). The reaction was stirred at 90° C. for 1 hour under N₂ atmosphere. The mixture was diluted with water (10 mL) and extracted with ethyl acetate (2×5 mL). The organic layers were dried over Na₂SO₄, concentrated and purified with reversed phase flash chromatography [C18, 0.1% NH₃.H₂O condition] to afford the title compound (40.0 mg, 31% yield) as yellow solid; LCMS (ESI, M+1): m/z=846.3.

Step E. 4-(4-((1R,5S)-3,8-diazabicyclo[3.2.1]octan-3-yl)-6-cyclopropyl-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of tert-butyl tert-butyl (1R,5S)-3-(7-(2-((tert-butoxycarbonyl)amino)-3-cyano-7-fluorobenzo[b]thiophen-4-yl)-6-cyclopropyl-8-fluoro-2-(((2R,7aS)-2-fluorotetrahydro-1H-pyrrolizin-7a(5H)-yl)methoxy)quinazolin-4-yl)-3,8-diazabicyclo[3.2.1]octane-8-carboxylate (40 mg, 1.0 equiv) in DCM (1 mL) was added TFA (1.54 g, 284 equiv). The mixture was stirred at 25° C. for 1 hour. The mixture was diluted with water (5 mL), and then the mixture was adjusted to pH=7 with saturated NaHCO₃(5 mL) and extracted with ethyl acetate (2×5 mL). The organic layers were dried over Na₂SO₄, concentrated and purified with prep-HPLC[Waters Xbridge 150×25 mm×5 μm; A: water (ammonia hydroxide v/v); B: ACN; B %: 40%-70% over 10 min] followed by prep-HPLC [Welch Xtimate C18 150×25 mm×5 μm; A: water (FA), B: ACN, B %: 5%-35% over 10 min] to afford the title compound (5.54 mg, 17% yield, HCOOH salt) as white solid; ¹H NMR (400 MHz, METHANOL-d4) δ=7.32 (s, 1H), 7.23 (m, 1H), 7.05 (dd, J=8.4, 9.2 Hz, 1H), 5.59-5.28 (m, 1H), 4.59-4.30 (m, 4H), 3.90 (br s, 2H), 3.80-3.50 (m, 5H), 3.27-3.16 (m, 1H), 2.52-2.12 (m, 5H), 1.97 (br s, 5H), 1.70-1.55 (m, 1H), 0.89-0.59 (m, 4H); LCMS (ESI, M+1): m/z=646.4.

### Example 209

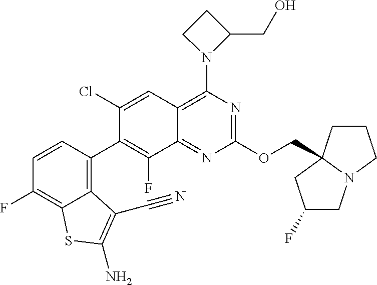 [SMILES: N#Cc1c(N)sc2c(F)ccc(-c3c(Cl)cc4c(N5CCC5CO)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(2-(hydroxymethyl)azetidin-1-yl)quinazolin-7-yl)-7-fluorobenzo[b]thiophene-3-carbonitrile

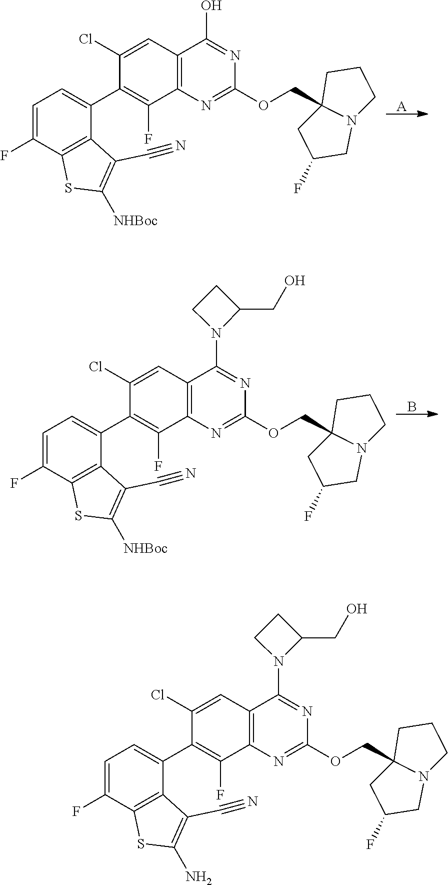

Step A. tert-butyl (4-(6-chloro-8-fluoro-2-(((2R,7aS)-2-fluorotetrahydro-1H-pyrrolizin-7a(5H)-yl)methoxy)-4-(2-(hydroxymethyl)azetidin-1-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100 mg, 1.0 equiv) in DMSO (1 mL) were added PyBOP (242 mg, 3.0 equiv) and TEA (47.0 mg, 3.0 equiv). The reaction was stirred at 25° C. for 1 hour. azetidin-2-ylmethanol (40.1 mg, 3.0 equiv) was added. The reaction was stirred at 25° C. for 12 hours. The mixture was diluted with water (10 mL) and extracted with ethyl acetate (2×10 mL). The organic layers were washed with brine (10 mL), dried over anhydrous sodium sulfate, concentrated and purified with reversed phase flash [C18, 0.1% formic acid condition] to afford the title compound (70.0 mg, 63% yield) as yellow solid; LCMS (ESI, M+1): m/z=715.2.

Step B. 2-amino-4-(6-chloro-8-fluoro-2-(((2R,7aS)-2-fluorotetrahydro-1H-pyrrolizin-7a(5H)-yl)methoxy)-4-(2-(hydroxymethyl)azetidin-1-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(2-(hydroxymethyl)azetidin-1-yl)quinazolin-7-yl)-3-cyano-7-fluorobenzo[b]thiophen-2-yl)carbamate (40.0 mg, 1.0 equiv) in DCM (0.3 mL) was added TFA (0.8 mL) at 0° C. The reaction was stirred at 0° C. for 1 hour. The mixture was adjusted to pH=7 with saturated NaHCO₃(10 mL) and extracted with DCM (2×10 mL). The combined organic layers were dried over anhydrous sodium sulfate, concentrated and purified by prep-HPLC [column: Waters Xbridge 150×25 mm×5 μm; A: water (ammonia hydroxide v/v), B: ACN, B %: 38%-68% over 10 min] to afford the title compound (22.1 mg, 64% yield) as white solid; ¹H NMR (400 MHz, METHANOL-d₄) δ=7.87-7.76 (m, 1H), 7.24-7.14 (m, 1H), 7.08-6.98 (m, 1H), 5.36-5.21 (m, 1H), 4.79-4.69 (m, 1H), 4.32-4.10 (m, 3H), 3.84 (br dd, J=3.6, 11.6 Hz, 1H), 3.30 (br s, 2H), 3.27-3.12 (m, 3H), 3.04-2.94 (m, 1H), 2.60 (br dd, J=7.2, 9.2 Hz, 1H), 2.54-2.43 (m, 1H), 2.37-2.16 (m, 2H), 2.15-2.07 (m, 1H), 2.00-1.82 (m, 3H); LCMS (ESI, M+1): m/z=615.2.

### Example 210

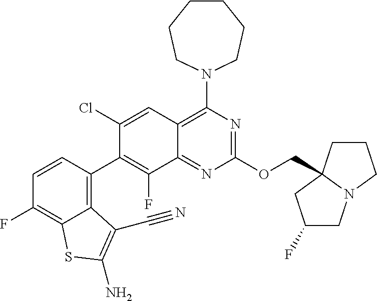 [SMILES: N#Cc1c(N)sc2c(F)ccc(-c3c(Cl)cc4c(N5CCCCCC5)nc(OC[C@@]56CCCN5C[C@H](F)C6)nc4c3F)c12]

### 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

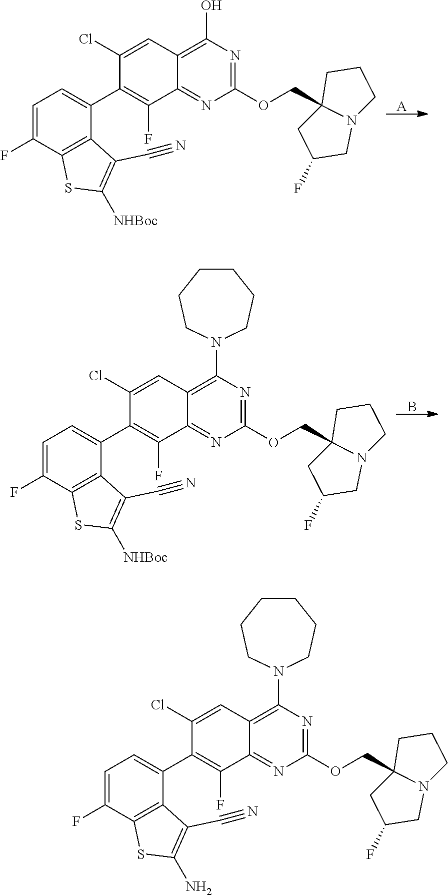

Step A. tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (60.0 mg, 1.0 equiv), PYBOP (72.5 mg, 1.5 equiv) and DIEA (36.0 mg, 3.0 equiv) in DMSO (1.00 mL). The reaction was stirred at 30° C. for 0.5 hours. Then the mixture was added azepane (50.0 mg, 5.4 equiv). The mixture was stirred at 30° C. for 1 hour. The mixture was diluted with water (10 mL) and extracted with EtOAc (2×15 mL). The combined organic layers were dried over anhydrous sodium sulfate, concentrated and purified with reversed phase flash chromatography [C18, 0.1% NH₃.H₂O condition] to afford the title compound (30.0 mg, 39% yield) as yellow solid; LCMS (ESI, M+1): m/z=727.3.

Step B. 2-amino-4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(4-(azepan-1-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (30.0 mg, 1.0 equiv) in DCM (0.50 mL) was added TFA (767 mg, 163 equiv). The reaction was stirred at 25° C. for 1 hour. The mixture was diluted with water (5 mL), and then the mixture was adjusted to pH=7 with saturated NaHCO₃(5 mL) and extracted with ethyl acetate (2×10 mL). The organic layers were dried over Na₂SO₄, concentrated and purified with prep-HPLC [Welch Xtimate C18 150×25 mm×5 μm; A: water (0.1% FA); B: ACN; B %: 23%-53% over 10 min] to afford the title compound (7.77 mg, 27% yield, HCOOH salt) as white solid; ¹H NMR (400 MHz, METHANOL-d4) δ=8.04 (s, 1H), 7.20 (dd, J=5.2, 8.4 Hz, 1H), 7.03 (t, J=9.2 Hz, 1H), 5.51-5.24 (m, 1H), 4.47-4.24 (m, 2H), 4.04 (br t, J=5.6 Hz, 4H), 3.62-3.35 (m, 3H), 3.21-3.04 (m, 1H), 2.56-2.15 (m, 3H), 2.13-1.86 (m, 7H), 1.70 (br s, 4H); LCMS (ESI, M+1): m/z=627.2.

### Example 211

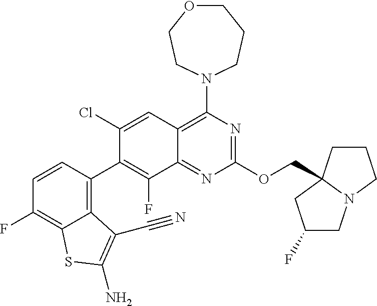 [SMILES: N#Cc1c(N)sc2c(F)ccc(-c3c(Cl)cc4c(N5CCCOCC5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile

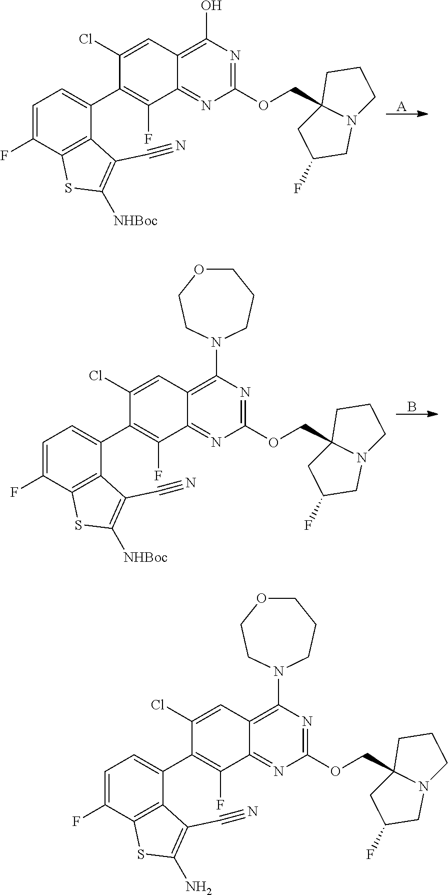

Step A. tert-butyl (4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (50.0 mg, 1.0 equiv) and DIEA (30.0 mg, 3.0 equiv) in DMSO (1.00 mL) was added PYBOP (60.4 mg, 1.5 equiv). The reaction was stirred at 30° C. for 0.5 hours. 1,4-oxazepane (15.7 mg, 2.0 equiv) was added to the mixture. The reaction was stirred at 30° C. for 1 hour. The reaction mixture was diluted with water (10 mL) and extracted with EtOAc (2×5 mL). The combined organic layers were dried over anhydrous sodium sulfate, and concentrated under vacuum to afford the title compound (70.0 mg, crude) as yellow solid; LCMS (ESI, M+1): m/z=729.4.

Step B. 2-amino-4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(1,4-oxazepan-4-yl)quinazolin-7-yl)-3-cyano-7-fluorobenzo[b]thiophen-2-yl)carbamate (65.0 mg, 1.0 equiv) in DCM (1 mL) was added TFA (1.54 g, 151 equiv). The reaction was stirred at 25° C. for 1 hour. The mixture was diluted with water (5 mL), and then the mixture was adjusted to pH=7 with saturated NaHCO₃(5 mL) and extracted with ethyl acetate (2×10 mL). The organic layers were dried over Na₂SO₄, concentrated and purified with prep-HPLC [Waters Xbridge 150×25 mm×5 μm; A: water (NH₄HCO₃); B: ACN; B %: 48%-78% over 9 min] followed by prep-HPLC [Phenomenex Luna C18 250×50 mm×15 μm; A: water (FA), B: ACN, B %: 22%-52% over 9 min] to afford the title compound (14.6 mg, 24% yield, HCOOH salt) as white solid; ¹H NMR (400 MHz, METHANOL-d4) δ=8.01 (d, J=1.2 Hz, 1H), 7.20 (dd, J=5.0, 8.2 Hz, 1H), 7.03 (t, J=8.8 Hz, 1H), 5.48-5.21 (m, 1H), 4.38-4.21 (m, 2H), 4.20-4.11 (m, 4H), 4.01 (t, J=4.6 Hz, 2H), 3.83 (t, J=5.0 Hz, 2H) 3.46-3.32 (m, 2H), 3.24 (br d, J=4.0 Hz, 1H), 3.13-3.01 (m, 1H), 2.47-2.13 (m, 5H), 2.10-1.82 (m, 3H); LCMS (ESI, M+1): m/z=629.3.

### Example 212

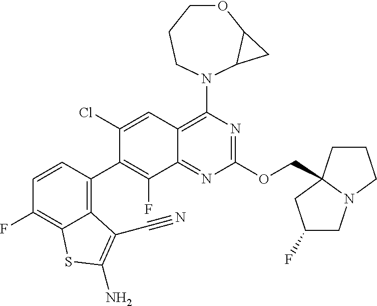 [SMILES: N#Cc1c(N)sc2c(F)ccc(-c3c(Cl)cc4c(N5CCCOC6CC65)nc(OC[C@@]56CCCN5C[C@H](F)C6)nc4c3F)c12]

### 4-(4-(2-oxa-6-azabicyclo[5.1.0]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (isomer 1)

### Example 213

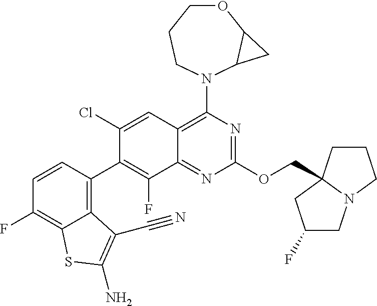 [SMILES: N#Cc1c(N)sc2c(F)ccc(-c3c(Cl)cc4c(N5CCCOC6CC65)nc(OC[C@@]56CCCN5C[C@H](F)C6)nc4c3F)c12]

### 4-(4-(2-oxa-6-azabicyclo[5.1.0]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile (isomer 2)

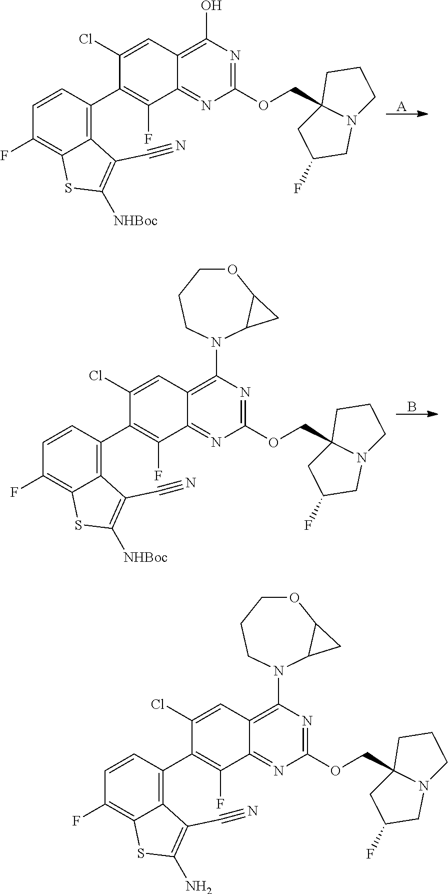

Step A: tert-butyl (4-(4-(2-oxa-6-azabicyclo[5.1.0]octan-6-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a mixture of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (141 mg, 2.0 equiv, TFA), PYBOP (241 mg, 1.5 equiv) and DIEA (320 mg, 8.0 equiv) in DMSO (1.0 mL) at 25° C. for 0.5 hours. The mixture was added tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (200 mg, 1.0 equiv) at 25° C. for 1 hour. The mixture was diluted with water (4 mL) and extracted with ethyl acetate (6 mL). The organic layer was dried over sodium sulfate, filtered and concentrated and purified by reversed phase flash chromatography [C18, 0.1% formic acid condition] to afford the title compound (180 mg, 60% yield) as a yellow solid; LCMS (ESI, M+1): m/z=741.4.

Step B. 4-(4-(2-oxa-6-azabicyclo[5.1.0]octan-6-yl)-6-chloro-8-fluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: To a solution of tert-butyl (4-(4-(2-oxa-6-azabicyclo[5.1.0]octan-6-yl)-6-chloro-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (150 mg, 1.0 equiv) in DCM (2.0 mL) was added TFA (2.30 g, 100 equiv). The reaction was stirred at 25° C. for 1 hour. The mixture was diluted with water (2 mL) and extracted with ethyl acetate (3 mL). The organic layer was dried over sodium sulfate, filtered and concentrated and purified by prep-HPLC [column: Waters Xbridge C18 150×25 mm×5 μm; A: water (ammonia hydroxide v/v), B: ACN; B %: 40%-70% over 9 min] to afford two isomers:

Isomer 1 (10.0 mg, 7.60% yield) as a yellow solid; ¹H NMR (400 MHz, METHANOL-d4) δ=8.34 (s, 1H), 7.23 (dd, J=5.2, 8.4 Hz, 1H), 7.08-6.99 (m, 1H), 5.40-5.19 (m, 1H), 4.60 (br d, J=13.6 Hz, 1H), 4.33-4.13 (m, 2H), 3.98 (br d, J=11.2 Hz, 1H), 3.87-3.78 (m, 1H), 3.74 (td, J=4.4, 6.4 Hz, 1H), 3.64 (br t, J=12.4 Hz, 1H), 3.34 (br s, 1H), 3.29-3.12 (m, 3H), 3.05-2.96 (m, 1H), 2.53-2.40 (m, 1H), 2.38-2.19 (m, 2H), 2.18-2.08 (m, 1H), 2.04-1.93 (m, 2H), 1.92-1.82 (m, 2H), 1.35 (q, J=7.2 Hz, 1H), 0.71 (ddd, J=4.0, 5.6, 7.6 Hz, 1H) LCMS (ESI, M+1): m/z=641.3.

Isomer 2 (10.0 mg, 7.60% yield) as a yellow solid; ¹H NMR (400 MHz, METHANOL-d4) δ=8.30 (d, J=1.5 Hz, 1H), 7.20 (dd, J=5.2, 8.4 Hz, 1H), 7.07-6.97 (m, 1H), 5.43-5.18 (m, 1H), 4.62 (br d, J=13.6 Hz, 1H), 4.33-4.24 (m, 1H), 4.23-4.15 (m, 1H), 4.01-3.91 (m, 1H), 3.87-3.75 (m, 1H), 3.71 (td, J=4.4, 6.4 Hz, 1H), 3.63 (br t, J=12.4 Hz, 1H), 3.36-3.32 (m, 1H), 3.27-3.11 (m, 3H), 3.06-2.94 (m, 1H), 2.51-2.41 (m, 1H), 2.40-2.25 (m, 1H), 2.24-2.18 (m, 1H), 2.17-2.08 (m, 1H), 2.03-1.93 (m, 2H), 1.92-1.79 (m, 2H), 1.30 (q, J=7.6 Hz, 1H), 0.87-0.80 (m, 1H) LCMS (ESI, M+1): m/z=641.2.

### Example 214

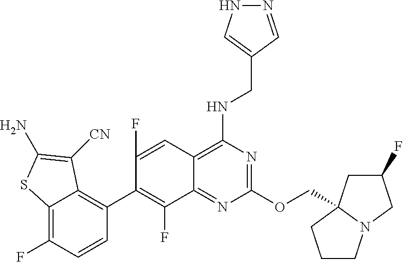 [SMILES: N#Cc1c(N)sc2c(F)ccc(-c3c(F)cc4c(NCc5cn[nH]c5)nc(OC[C@@]56CCCN5C[C@H](F)C6)nc4c3F)c12]

### 4-(4-(((1H-pyrazol-4-yl)methyl)amino)-6,8-difluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile

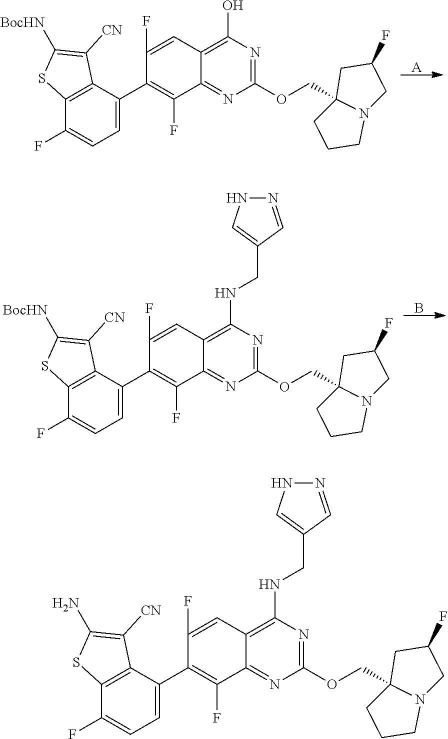

Step A. tert-butyl (4-(4-(((1H-pyrazol-4-yl)methyl)amino)-6,8-difluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (3-cyano-4-(6,8-difluoro-2-(((2R,7aS)-2-fluorotetrahydro-1H-pyrrolizin-7a(5H)-yl)methoxy)-4-hydroxyquinazolin-7-yl)-7-fluorobenzo[b]thiophen-2-yl)carbamate (70.0 mg, 1.0 qeuiv) and DIEA (43.1 mg, 3.0 equiv) in DMSO (1.00 mL) was added PYBOP (116 mg, 2.0 equiv). After stirring at 25° C. for 0.5 hours, (1H-pyrazol-4-yl)methanaminemethenamine (22.3 mg, 1.50 equiv, HCl) was added to the mixture. The reaction was stirred at 25° C. for 1 hour. The mixture was diluted with water (1.00 mL) and extracted with ethyl acetate (3×3 mL). The combined organic layers were dried over Na₂SO₄, filtered, concentrated and purified by reversed-phase flash chromatography (C18, 0.1%, FA) to the title compound as a white solid; ¹H NMR (400 MHz, DMSO-d₆) δ=12.90-12.53 (m, 1H), 8.81 (br d, J=1.2 Hz, 1H), 8.04 (br d, J=10.0 Hz, 1H), 7.77-7.55 (m, 2H), 7.40-7.26 (m, 1H), 7.23-7.09 (m, 1H), 5.54-5.32 (m, 1H), 4.62 (br d, J=4.6 Hz, 2H), 4.38-4.24 (m, 2H), 3.51-3.44 (m, 2H), 3.11-3.01 (m, 1H), 2.30-2.15 (m, 2H), 2.10-1.82 (m, 4H), 1.45 (s, 11H).

Step B. 4-(4-(((1H-pyrazol-4-yl)methyl)amino)-6,8-difluoro-2-(((2R,7aS)-2-fluorotetrahydro-1H-pyrrolizin-7a(5H)-yl)methoxy)quinazolin-7-yl)-2-amino-7-fluorobenzo[b]thiophene-3-carbonitrile: A solution of tert-butyl (4-(4-(((1H-pyrazol-4-yl)methyl)amino)-6,8-difluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (25.0 mg, 1.0 equiv) in HCl.MeOH (2 M, 17.6 μL, 1.0 equiv) was stirred at 25° C. for 3 hours. The mixture was concentrated, dissolved in MeOH (2 mL), adjusted pH to 7 with solid NaHCO₃ and filtered. The filtrate was concentrated and purified by prep-HPLC [Waters xbridge 150×25 mm×5 um; A: water (NH₄HCO₃), B: ACN; B: 33%-63% B over 10 min] to afford the title compound (5.84 mg, 27.0% yield) as a white solid; ¹H NMR (400 MHz, DMSO-d₆) δ=12.71 (br s, 1H), 8.70 (br t, J=5.2 Hz, 1H), 8.13 (s, 2H), 8.03 (br d, J=9.6 Hz, 1H), 7.76-7.66 (m, 1H), 7.54 (br d, J=2.0 Hz, 1H), 7.30 (dd, J=5.6, 8.0 Hz, 1H), 7.14 (t, J=8.8 Hz, 1H), 5.37-5.19 (m, 1H), 4.67-4.55 (m, 2H), 4.15-4.06 (m, 1H), 4.05-4.00 (m, 1H), 3.10 (br d, J=11.6 Hz, 2H), 3.02 (br s, 1H), 2.89-2.80 (m, 1H), 2.21-2.10 (m, 1H), 2.07-2.00 (m, 2H), 1.88-1.73 (m, 3H); LCMS (ESI, M+1): m/z=609.1.

### Example 215

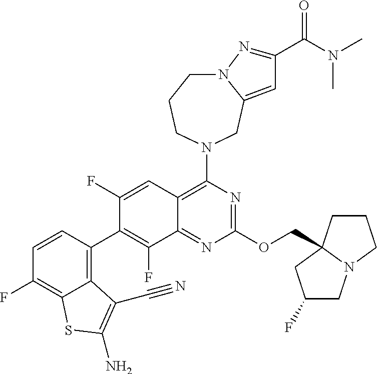 [SMILES: CN(C)C(=O)c1cc2n(n1)CCCN(c1nc(OC[C@@]34CCCN3C[C@H](F)C4)nc3c(F)c(-c4ccc(F)c5sc(N)c(C#N)c45)c(F)cc13)C2]

### 5-(7-(2-amino-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide

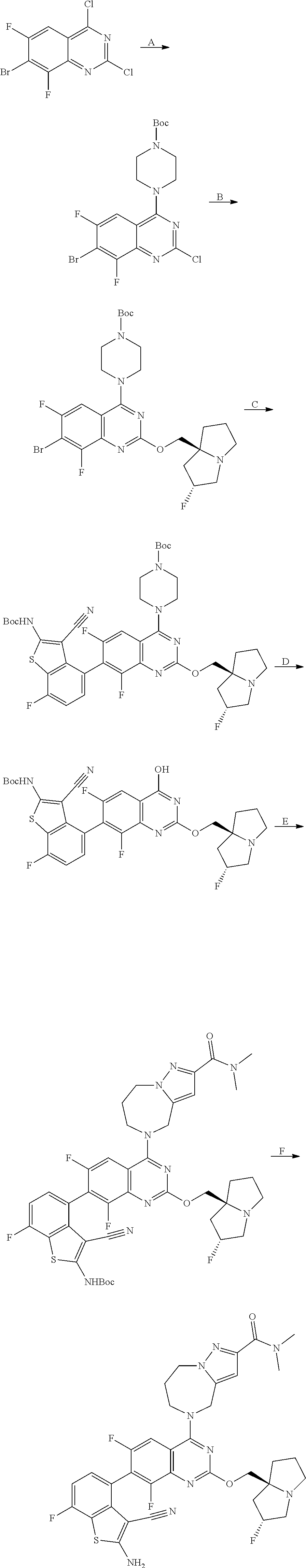

Step A. tert-butyl 4-(7-bromo-2-chloro-6,8-difluoroquinazolin-4-yl)piperazine-1-carboxylate: To a solution of 7-bromo-2,4-dichloro-6,8-difluoroquinazoline (1.40 g, 1.0 equiv) in DCM (15 mL) was added DIEA (1.73 g, 3.0 equiv) at −40° C. To the mixture was added tert-butyl piperazine-1-carboxylate (831 mg, 1.0 equiv). The reaction was stirred at −40° C. for 1 hour. The mixture was quenched by water (20 mL) at 0° C. and extracted with ethyl acetate (3×10 mL). The combined organic phase was dried over anhydrous sodium sulfate, concentrated and triturated with ACN at 25° C. for 15 minutes to afford the title compound (1.87 g, 82% yield) as a yellow solid; LCMS (ESI, M+1, M+3): m/z=463.0, 465.0.

Step B. tert-butyl 4-(7-bromo-6,8-difluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-bromo-2-chloro-6,8-difluoroquinazolin-4-yl)piperazine-1-carboxylate (1.60 g, 1.0 equiv) and ((2R,7aS)-2-fluorotetrahydro-1H-pyrrolizin-7a(5H)-yl)methanol (824 mg, 1.5 equiv) and DABCO (387 mg, 1.0 equiv) in THE (5.0 mL) and DMF (5.0 mL) were added Cs₂CO₃ (3.37 g, 3.0 equiv) and 4 Å MS (300 mg, 1,0 equiv). The reaction was stirred at 25° C. for 4 hours. The mixture was diluted with water (10 mL) and extracted with ethyl acetate (3×20 mL). The combined organic layers were dried over anhydrous sodium sulfate, filtered and concentrated under reduced pressure and purified by reversed phase flash chromatography [C18, 0.1% formic acid condition] to afford the title compound (600 mg, 28% yield) as a yellow solid; LCMS (ESI, M+1, M+3): m/z=586.2, 588.2

Step C. tert-butyl 4-(7-(2-((tert-butoxycarbonyl)amino)-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)piperazine-1-carboxylate: To a solution of tert-butyl 4-(7-bromo-6,8-difluoro-2-(((2R,7aS)-2-fluorotetrahydro-1H-pyrrolizin-7a(5H)-yl)methoxy)quinazolin-4-yl)piperazine-1-carboxylate (600 mg, 1.0 equiv) and tert-butyl (3-cyano-4-(5,5-dimethyl-1,3,2-dioxaborinan-2-yl)-7-fluorobenzo[b]thiophen-2-yl)carbamate (620 mg, 1.5 equiv) in THE (8.0 mL) were added Cs₂CO₃ (1.00 g, 3.0 equiv) and dichloro[bis(2-(diphenylphosphino)phenyl)ether]palladium(II) (70.4 mg, 0.10 equiv). The reaction was stirred at 60° C. for 12 hours. The mixture was diluted with water (20 mL) and extracted with ethyl acetate (3×20 mL). The combined organic layers were dried over anhydrous sodium sulfate, filtered, concentrated under reduced pressure and purified by reversed phase flash chromatography [C18, 0.1% formic acid condition] to afford the title compound (485 mg, 53% yield) as a yellow solid; LCMS (ESI, M+1): m/z=798.7

Step D. tert-butyl (3-cyano-4-(6,8-difluoro-2-(((2R,7aS)-2-fluorotetrahydro-1H-pyrrolizin-7a(5H)-yl)methoxy)-4-hydroxyquinazolin-7-yl)-7-fluorobenzo[b]thiophen-2-yl)carbamate: To a solution of tert-butyl 4-(7-(2-((tert-butoxycarbonyl)amino)-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)piperazine-1-carboxylate (630 mg, 1.0 equiv) in EtOH (10 mL) was added NaOH (1 M, 7.90 mL, 10 equiv). The reaction was stirred at 60° C. for 12 hours. The mixture was adjusted pH to 4 with HCl (2 M), diluted with water (20 mL) and extracted with ethyl acetate (3×20 mL). The combined organic layers were dried over anhydrous sodium sulfate, filtered, concentrated under reduced pressure and purified by reversed phase flash chromatography [C18, 0.1% formic acid condition] to afford the title compound (440 mg, 80% yield) as a yellow solid; LCMS (ESI, M+1): m/z=630.2

Step E. tert-butyl (3-cyano-4-(4-(2-(dimethylcarbamoyl)-7,8-dihydro-4H-pyrazolo[1,5-a][1,4]diazepin-5(6H)-yl)-6,8-difluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate: To a solution of tert-butyl (3-cyano-4-(6,8-difluoro-2-(((2R,7aS)-2-fluorotetrahydro-1H-pyrrolizin-7a(5H)-yl)methoxy)-4-hydroxyquinazolin-7-yl)-7-fluorobenzo[b]thiophen-2-yl)carbamate (80.0 mg, 1.0 equiv) and DIEA (49.3 mg, 3.0 equiv) in DMSO (2.0 mL) was added PYBOP (132 mg, 2.0 equiv). The reaction was stirred at 25° C. for 0.5 hours. To the mixture was added N,N-dimethyl-5,6,7,8-tetrahydro-4H-pyrazolo[1,5-a][1,4]diazepine-2-carboxamide (40.0 mg, 1.5 eq). The reaction was stirred at 25° C. for 1 hour. The mixture was quenched by addition water (10 mL) at 25° C. and extracted with ethyl acetate (3×10 mL). The combined organic layers were dried over anhydrous sodium sulfate, filtered, concentrated under reduced pressure and purified by reversed phase flash chromatography [C18, 0.1% formic acid condition] to afford the title compound (65.0 mg, 56% yield) as a yellow solid; LCMS (ESI, M+1): m/z=820.5.

Step F. 5-(7-(2-amino-3-cyano-7-fluorobenzo[b]thiophen-4-yl)-6,8-difluoro-2-(((2R,7aS)-2-fluorotetrahydro-1H-pyrrolizin-7a(5H)-yl)methoxy)quinazolin-4-yl)-N,N-dimethyl-5,6,7,8-tetrahydro-4H-pyrazolo[1,5-a][1,4]diazepine-2-carboxamide: A solution of tert-butyl (3-cyano-4-(4-(2-(dimethylcarbamoyl)-7,8-dihydro-4H-pyrazolo[1,5-a][1,4]diazepin-5(6H)-yl)-6,8-difluoro-2-(((2R,7aS)-2-fluorotetrahydro-1H-pyrrolizin-7a(5H)-yl)methoxy)quinazolin-7-yl)-7-fluorobenzo[b]thiophen-2-yl)carbamate (60.0 mg, 1.0 equiv) in HCl/MeOH (2 M, 2 mL, 54.0 equiv) was stirred at 25° C. for 16 hours. The mixture was concentrated and purified by Prep-HPLC [Phenomenex luna C18 150×25 mm×10 um; A: water (FA), B: ACN; B %:17%-37% over 10 min] to afford the title compound (22.2 mg, 39% yield, HCOOH) as a white solid; ¹H NMR (400 MHz, METHANOL-d₄) δ=7.68 (br d, J=10.0 Hz, 1H), 7.28 (dd, J=5.0, 8.4 Hz, 1H), 7.08-6.99 (m, 1H), 6.71 (s, 1H), 5.44-5.24 (m, 1H), 5.09 (s, 2H), 4.93 (s, 1H), 4.64-4.40 (m, 3H), 4.35-4.21 (m, 4H), 3.52-3.35 (m, 2H), 3.33 (s, 3H), 3.08 (s, 3H), 2.44-2.31 (m, 3H), 2.30-2.23 (m, 1H), 2.21-2.13 (m, 1H), 2.11-1.99 (m, 2H), 1.99-1.88 (m, 1H); LCMS (ESI, M+1): m/z=720.0.

### Example 216

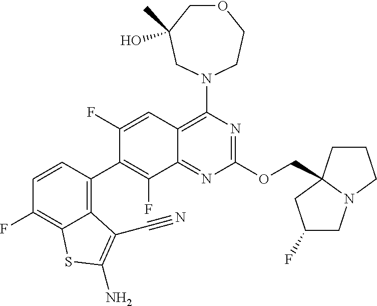 [SMILES: C[C@@]1(O)COCCN(c2nc(OC[C@@]34CCCN3C[C@H](F)C4)nc3c(F)c(-c4ccc(F)c5sc(N)c(C#N)c45)c(F)cc23)C1]

### 2-amino-4-(6,8-difluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile

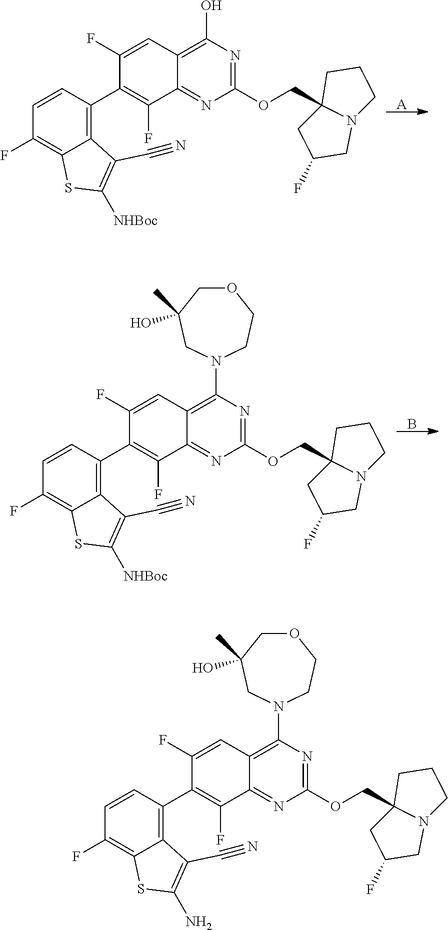

Step A. tert-butyl (3-cyano-4-(6,8-difluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophen-2-yl)carbamate: To a solution of tert-butyl (3-cyano-4-(6,8-difluoro-2-(((2R,7aS)-2-fluorotetrahydro-1H-pyrrolizin-7a(5H)-yl)methoxy)-4-hydroxyquinazolin-7-yl)-7-fluorobenzo[b]thiophen-2-yl)carbamate (70.0 mg, 1.0 equiv) and DIEA (43.1 mg, 3.0 equiv) in DMSO (1.00 mL) was added PYBOP (86.8 mg, 1.5 equiv). After stirring at 20° C. for 0.5 hours, (6S)-6-methyl-1,4-oxazepan-6-ol (27.9 mg, 1.5 equiv, HCl) was added to the mixture. The reaction was stirred at 20° C. for 1 hour. The mixture was diluted with water (5.0 mL) and extracted with ethyl acetate (3×5.0 mL). The combined organic layers were dried over anhydrous sodium sulfate, concentrated and purified by reversed-phase flash chromatography (C18, 0.1% FA) to afford the title compound (50.0 mg, 51.5% yield) as a brown solid. LCMS (ESI, M+1): m/z=743.0.

Step B. 2-amino-4-(6,8-difluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophene-3-carbonitrile: A solution of tert-butyl (3-cyano-4-(6,8-difluoro-2-(((2R,7aS)-2-fluorotetrahydro-1H-pyrrolizin-7a(5H)-yl)methoxy)-4-((S)-6-hydroxy-6-methyl-1,4-oxazepan-4-yl)quinazolin-7-yl)-7-fluorobenzo[b]thiophen-2-yl)carbamate (45.0 mg, 1.0 equiv) in HCl/MeOH (2 M, 0.5 mL) was stirred at 20° C. for 6 hours. The mixture was concentrated, diluted with MeOH (1.0 mL), adjusted pH to 6 by NaHCO₃ and filtered. The filtrate was concentrated and purified by reversed-phase HPLC(column: Waters xbridge 150×25 mm 10 um; mobile phase: [water(NH₄HCO₃)−ACN];gradient:322%-62% B over 10 min) to afford the title compound (12.7 mg, 32.28% yield) as a white solid; ¹H NMR (400 MHz, MeOD-d₄) δ=8.08-7.97 (m, 1H), 7.32-7.26 (m, 1H), 7.05 (t, J 8.8 Hz, 1H), 5.41-5.23 (m, 1H), 4.49-4.40 (m, 1H), 4.35-4.20 (m, 3H), 4.04-3.97 (m, 2H), 3.91-3.79 (m, 2H), 3.73-3.66 (m, 1H), 3.65-3.57 (m, 1H), 3.28-3.17 (m, 3H), 3.08-2.98 (m, 1H), 2.39-2.10 (m, 3H), 2.05-1.95 (m, 2H), 1.95-1.85 (m, 1H), 1.26 (s, 3H); LCMS (ESI, M+1): m/z=643.1.

### Example 217

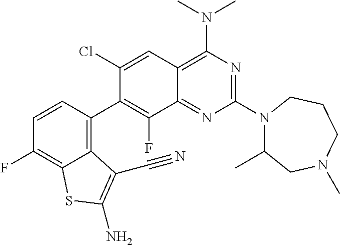 [SMILES: CC1CN(C)CCCN1c1nc(N(C)C)c2cc(Cl)c(-c3ccc(F)c4sc(N)c(C#N)c34)c(F)c2n1]

### 2-amino-4-(6-chloro-2-(2,4-dimethyl-1,4-diazepan-1-yl)-4-(dimethylamino)-8-fluoroquinazolin-7-yl)-7-fluorobenzo[b]thiophene-3-carbonitrile

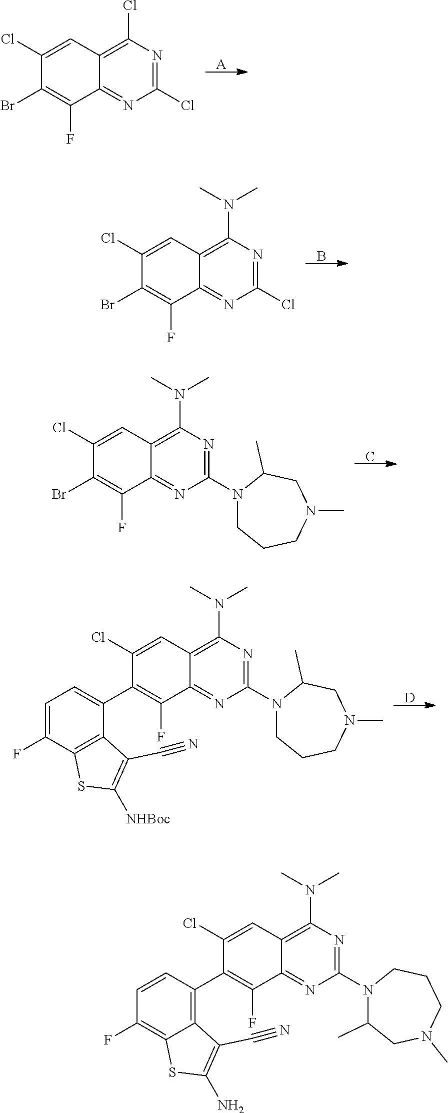

Step A. 7-bromo-2,6-dichloro-8-fluoro-N,N-dimethylquinazolin-4-amine: To a solution of 7-bromo-2,4,6-trichloro-8-fluoroquinazoline (500 mg, 1.0 equiv) and dimethylamine (185 mg, 1.5 equiv, HCl) in DCM (5 mL) was added DIEA (978 mg, 5.0 equiv). The reaction was stirred at −40° C. for 0.5 hours. The mixture was quenched with water (20 mL) and extracted with DCM (20 mL×2). The combined organic layers were washed with brine (20 mL), dried over anhydrous sodium sulfate, filtered and concentrated under reduced pressure to afford the title compound (500 mg, 96% yield) as off-white solid; LCMS (ESI, M+1, M+3): m/z=337.9, 339.9.

Step B. 7-bromo-6-chloro-2-(2,4-dimethyl-1,4-diazepan-1-yl)-8-fluoro-N,N-dimethylquinazolin-4-amine: To a solution of 7-bromo-2,6-dichloro-8-fluoro-N,N-dimethylquinazolin-4-amine (500 mg, 1.0 equiv) and 1,3-dimethyl-1,4-diazepane (189 mg, 1.0 equiv) in DMF (4 mL) was added DIEA (381 mg, 2.0 equiv). The reaction was stirred at 90° C. for 10 hours. The mixture was diluted with water (30 mL) and extracted with ethyl acetate (30 mL×2). The combined organic layers were washed with brine (30 mL), dried over anhydrous sodium sulfate, concentrated and purified with column chromatography [SiO₂, petroleum ether/ethyl acetate=I/O to 0/1] to afford the title compound (200 mg, 28% yield) as yellow solid; LCMS (ESI, M+1, M+3): m/z=430.0, 432.0.

Step C. tert-butyl (4-(6-chloro-2-(2,4-dimethyl-1,4-diazepan-1-yl)-4-(dimethylamino)-8-fluoroquinazolin-7-yl)-3-cyano-7-fluorobenzo[b]thiophen-2-yl)carbamate: To a solution of 7-bromo-6-chloro-2-(2,4-dimethyl-1,4-diazepan-1-yl)-8-fluoro-N,N-dimethylquinazolin-4-amine (100 mg, 1.0 equiv) and tert-butyl (3-cyano-4-(5,5-dimethyl-1,3,2-dioxaborinan-2-yl)-7-fluorobenzo[b]thiophen-2-yl)carbamate (113 mg, 1.2 equiv) in dioxane (2 mL) were added Cs₂CO₃ (151 mg, 2.0 equiv) and Pd(DPEphos)Cl₂ (16.0 mg, 0.1 equiv). The reaction was degassed and purged with N₂ for 3 times. The reaction was stirred at 90° C. for 1 hour under N₂ atmosphere. The mixture was diluted with water (10 mL) and extracted with ethyl acetate (2×10 mL). The combined organic layers were washed with brine (10 mL), dried over anhydrous sodium sulfate, concentrated and purified with reversed-phase HPLC (0.1% FA condition) to afford the title compound (56.0 mg, 36% yield) as yellow solid; LCMS (ESI, M+1): m/z=642.2.

Step D. 2-amino-4-(6-chloro-2-(2,4-dimethyl-1,4-diazepan-1-yl)-4-(dimethylamino)-8-fluoroquinazolin-7-yl)-7-fluorobenzo[b]thiophene-3-carbonitrile: To a solution of tert-butyl (4-(6-chloro-2-(2,4-dimethyl-1,4-diazepan-1-yl)-4-(dimethylamino)-8-fluoroquinazolin-7-yl)-3-cyano-7-fluorobenzo[b]thiophen-2-yl)carbamate (56.0 mg, 1.0 equiv) in MeCN (3 mL) was added HCl.dioxane (4 M, 3 mL). The reaction was stirred at 0° C. for 2 hours. The mixture was concentrated, dissolved in MeOH (2 mL), adjusted to pH=7 with solid NaHCO₃ and filtered. The filtrate was concentrated and purified with prep-HPLC [column: Waters Xbridge 150×25 mm×5 um; mobile phase: water (NH₄HCO₃)−ACN; gradient: 58%-88% B over 9 min] to afford the title compound (4.32 mg, 11% yield) as white solid, ¹H NMR (400 MHz, METHANOL-d₄) δ=7.85-7.80 (m, 1H), 7.21-7.12 (m, 1H), 7.01 (t, J=9.2 Hz, 1H), 4.99 (quind, J=5.6, 12.0 Hz, 1H), 4.56 (br d, J=7.2 Hz, 1H), 3.32 (s, 6H), 3.20-3.08 (m, 1H), 2.99 (dd, J=5.6, 14.4 Hz, 1H), 2.87 (br d, J=10.8 Hz, 1H), 2.65 (dd, J=11.6, 14.4 Hz, 1H), 2.55-2.43 (m, 1H), 2.35 (s, 3H), 2.07-1.91 (m, 1H), 1.74-1.64 (m, 1H), 1.13 (d, J=6.4 Hz, 3H); LCMS (ESI, M+1): m/z=542.2.

### Example 218

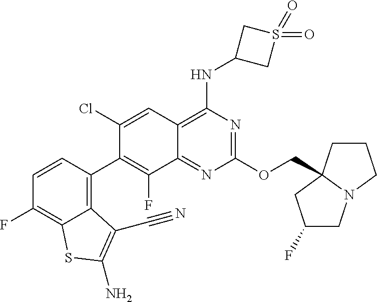 [SMILES: N#Cc1c(N)sc2c(F)ccc(-c3c(Cl)cc4c(NC5CS(=O)(=O)C5)nc(OC[C@@]56CCCN5C[C@H](F)C6)nc4c3F)c12]

### 2-amino-4-(6-chloro-4-((1,1-dioxidothietan-3-yl)amino)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

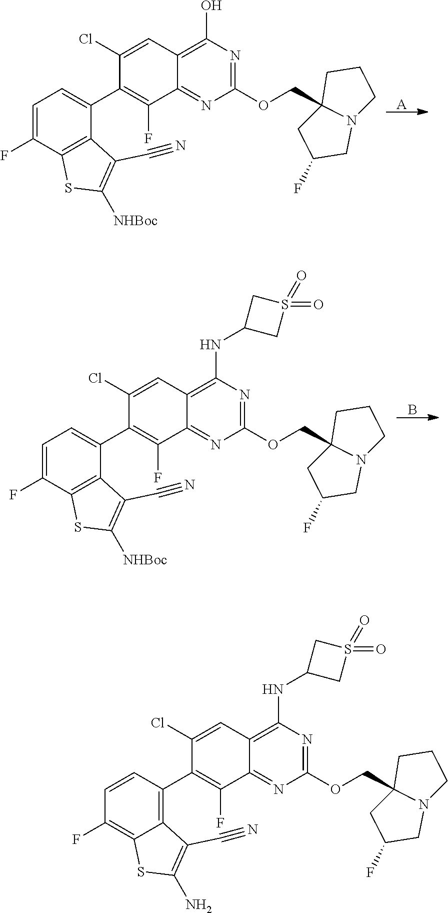

Step A. tert-butyl (4-(6-chloro-4-((1,1-dioxidothietan-3-yl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (100.0 mg, 1.0 equiv), PYBOP (121 mg, 1.5 equiv) and DIEA (60.0 mg, 3.0 equiv) in DMSO (2.00 mL) was added 3-aminothietane 1,1-dioxide (56.3 mg, 3.0 equiv). The reaction was stirred at 30° C. for 1 hour. The mixture was diluted with water (10 mL) and extracted with EtOAc (2×5 mL). The combined organic layers were dried over anhydrous sodium sulfate, concentrated and purified with reversed phase flash chromatography [C18, 0.1% NH₃.H₂O condition] to afford the title compound (80.0 mg, 64% yield) as yellow solid; LCMS (ESI, M+1): m/z=749.2.

Step B. 2-amino-4-(6-chloro-4-((1,1-dioxidothietan-3-yl)amino)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile: To a solution of tert-butyl (4-(6-chloro-4-((1,1-dioxidothietan-3-yl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate (40.0 mg, 1.0 equiv) in DCM (0.50 mL) was added TFA (767 mg, 126 equiv). The reaction was stirred at 25° C. for 0.5 hours. The mixture was diluted with water (5 mL), and then the mixture was adjusted to pH=7 with saturated NaHCO₃(5 mL) and extracted with ethyl acetate (2×5 mL). The organic layers were dried over Na₂SO₄, concentrated and purified with prep-HPLC [Waters Xbridge 150×25 mm×5 μm; A: water (ammonia hydroxide v/v); B: ACN; B %: 35%-65% over 10 min] to afford the title compound (4.47 mg, 13% yield) as white solid; ¹H NMR (400 MHz, METHANOL-d4) δ=8.22 (s, 1H), 7.20 (dd, J=5.2, 8.4 Hz, 1H), 7.10-6.97 (t, J=8.8 Hz, 1H), 5.42-5.21 (m, 1H), 5.01-4.90 (m, 2H), 4.70 (br dd, J=8.8, 14.4 Hz, 2H), 4.41 (br dd, J=4.6, 14.6 Hz, 2H), 4.32-4.14 (m, 2H), 3.27-3.17 (m, 2H), 3.11-2.95 (m, 1H), 2.40-2.10 (m, 3H), 2.07-1.82 (m, 3H); LCMS (ESI, M+1): m/z=649.2.

### Example 219

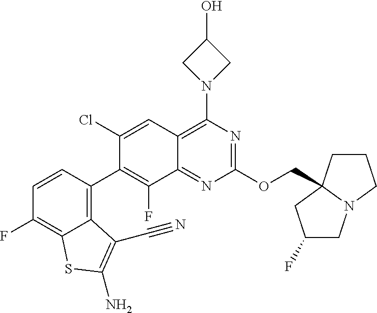 [SMILES: N#Cc1c(N)sc2c(F)ccc(-c3c(Cl)cc4c(N5CC(O)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(3-hydroxyazetidin-1-yl)quinazolin-7-yl)-7-fluorobenzo[b]thiophene-3-carbonitrile

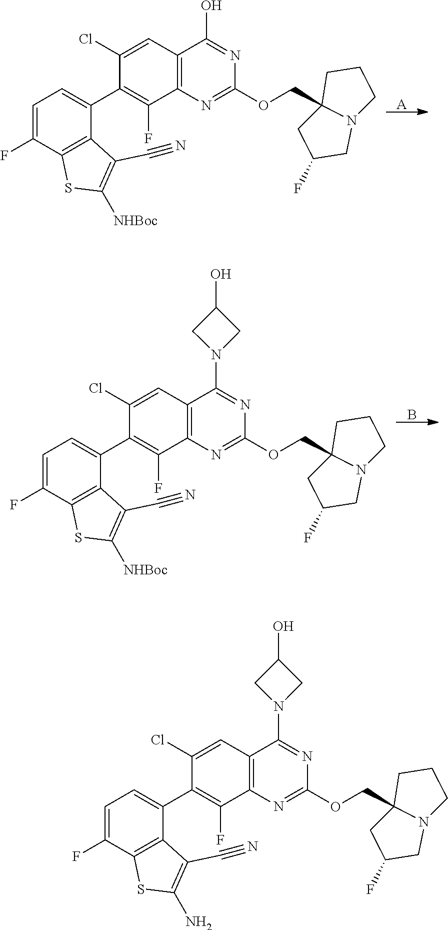

Step A. tert-butyl (4-(6-chloro-8-fluoro-2-(((2R,7aS)-2-fluorotetrahydro-1H-pyrrolizin-7a(5H)-yl)methoxy)-4-(3-hydroxyazetidin-1-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (90.0 mg, 1.0 equiv) in DMSO (1 mL) were added PyBOP (109 mg, 1.5 equiv) and TEA (113 mg, 8.0 equiv). The reaction was stirred at 30° C. for 0.5 hours. Azetidin-3-ol hydrogen chloride (92 mg, 6.0 equiv) was added into the mixture. The reaction was stirred at 30° C. for 12 hours. The mixture was filtered and purified with reversed phase flash [0.1% FA condition] to afford the title compound (60.0 mg, 55% yield) as yellow solid; LCMS (ESI, M+1): m/z=701.2.

Step B. 2-amino-4-(6-chloro-8-fluoro-2-(((2R,7aS)-2-fluorotetrahydro-1H-pyrrolizin-7a(5H)-yl)methoxy)-4-(3-hydroxyazetidin-1-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(3-hydroxyazetidin-1-yl)quinazolin-7-yl)-3-cyano-7-fluorobenzo[b]thiophen-2-yl)carbamate (50.0 mg, 1.0 equiv) in DCM (0.5 mL) was added TFA (1.54 g, 189 equiv) at 0° C. The reaction was stirred at 20° C. for 0.5 hours. The mixture was added into saturated Na₃HCO₃ aqueous (10 mL) at 0° C. and extracted with EtOAc (4×5 mL). The combined organic layers were washed with brine (5 mL), dried over anhydrous sodium sulfate, concentrated and purified with prep-HPLC [column: Phenomenex luna C18 150×25 mm×10 μm; mobile phase: water(FA)−ACN; gradient:16%-46% B over 10 minutes] to afford the title compound (12.1 mg, 28% yield) as white solid; ¹H NMR (400 MHz, METHANOL-d4) δ=7.82 (s, 1H), 7.20 (dd, J=5.2, 8.4 Hz, 1H), 7.04 (t, J=8.8 Hz, 1H), 5.51-5.26 (m, 1H), 4.84-4.74 (m, 3H), 4.53-4.22 (m, 4H), 3.62-3.37 (m, 3H), 3.17-3.15 (m, 1H), 2.52-2.28 (m, 2H), 2.28-1.89 (m, 4H); LCMS (ESI, M+1): m/z=601.2.

### Example 220

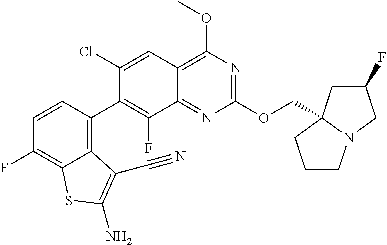 [SMILES: COc1nc(OC[C@@]23CCCN2C[C@H](F)C3)nc2c(F)c(-c3ccc(F)c4sc(N)c(C#N)c34)c(Cl)cc12]

### 2-amino-4-(6-chloro-8-fluoro-2-(((2R,7aS)-2-fluorotetrahydro-1H-pyrrolizin-7a(5H)-yl)methoxy)-4-methoxyquinazolin-7-yl)-7-fluorobenzo[b]thiophene-3-carbonitrile

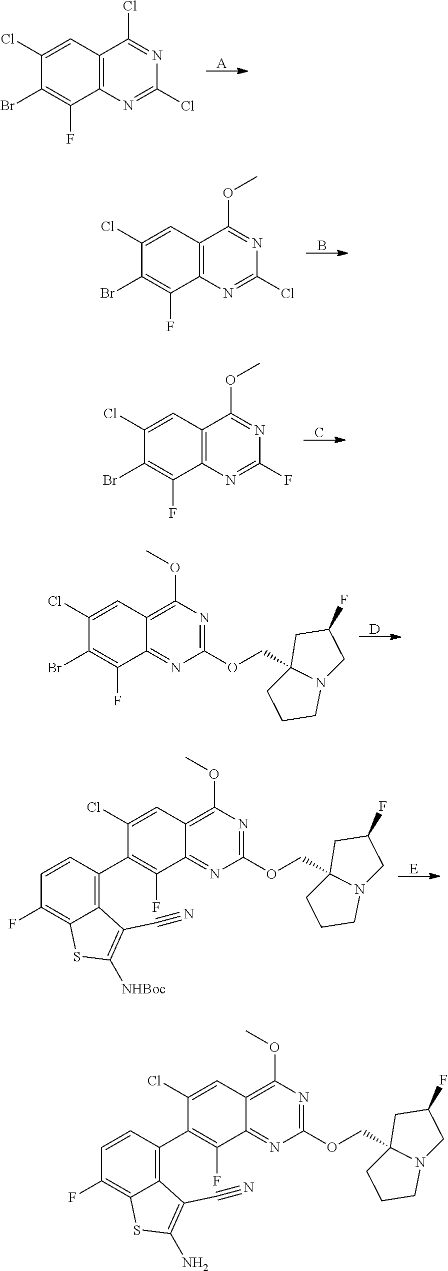

Step A. 7-bromo-2,6-dichloro-8-fluoro-4-methoxyquinazoline: To a solution of 7-bromo-2,4,6-trichloro-8-fluoroquinazoline (5.00 g, 1.0 equiv) in methanol (5 mL) was added sodium methoxide (0.980 g, 1.2 equiv) at 0° C. The reaction was stirred at 25° C. for 4 hours. The mixture was concentrated and purified by silica gel column chromatography (petroleum ether/ethyl acetate=I/O to 10/1) to afford the title compound (4.3 g, 87% yield) as pale yellow solid.

Step B. 7-bromo-6-chloro-2,8-difluoro-4-methoxyquinazoline: To a mixture of KF (178 mg, 10.0 equiv) and 7-bromo-2,6-dichloro-8-fluoro-4-methoxyquinazoline (100 mg, 1.0 equiv) in DMSO (1 mL) was added 18-crown-6 (8.11 mg, 0.1 equiv). The reaction was stirred at 120° C. for 2 hours. The mixture was filtered and purified by reversed-phase flash chromatography (0.1% FA condition) to afford the title compound (80 mg, 84% yileld) as yellow solid.

Step C. 7-bromo-6-chloro-8-fluoro-2-(((2R,7aS)-2-fluorotetrahydro-1H-pyrrolizin-7a(5H)-yl)methoxy)-4-methoxyquinazoline: To a solution of ((2R,7aS)-2-fluorotetrahydro-1H-pyrrolizin-7a(5H)-yl)methanol (113 mg, 1.1 equiv) in toluene (4.00 mL) was added t-BuONa (93.2 mg, 1.5 equiv) slowly at 0° C. The reaction was stirred at 25° C. for 10 minutes. 7-bromo-6-chloro-2,8-difluoro-4-methoxyquinazoline (200 mg, 1.0 equiv) was added slowly at 0° C. The reaction was stirred at 25° C. for 0.5 hours. The mixture was filtered and the filter cake was triturated with petroleum ether/ethyl acetate (5:1, 5 mL) at 25° C. for 20 minutes to afford the title compound (160 mg, 55% yield) as white solid; LCMS (ESI, M+1): m/z=449.9.

Step D. tert-butyl (4-(6-chloro-8-fluoro-2-(((2R,7aS)-2-fluorotetrahydro-1H-pyrrolizin-7a(5H)-yl)methoxy)-4-methoxyquinazolin-7-yl)-3-cyano-7-fluorobenzo[b]thiophen-2-yl)carbamate: To a solution of tert-butyl (3-cyano-4-(5,5-dimethyl-1,3,2-dioxaborinan-2-yl)-7-fluorobenzo[b]thiophen-2-yl)carbamate (100 mg, 1.0 equiv) and 7-bromo-6-chloro-8-fluoro-2-(((2R,7aS)-2-fluorotetrahydro-1H-pyrrolizin-7a(5H)-yl)methoxy)-4-methoxyquinazoline (111 mg, 1.0 equiv) in THE (5.00 mL) were added Cs₂CO₃ (242 mg, 3.0 equiv) and CataCXium A Pd G3 (18.0 mg, 0.1 equiv). The reaction was degassed and purged with nitrogen 3 times. The reaction was stirred at 70° C. for 1 hour. The mixture was filtered, concentrated and purified by prep-HPLC [Waters Xbridge 150×25 mm×5 μm; A: water (NH₄HCO₃), B: ACN, B %: 65%-95% over 9 min] to afford the title compound (20.0 mg, 12% yield) as yellow solid; LCMS (ESI, M+1): m/z=660.3.

Step E. 2-amino-4-(6-chloro-8-fluoro-2-(((2R,7aS)-2-fluorotetrahydro-1H-pyrrolizin-7a(5H)-yl)methoxy)-4-methoxyquinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-methoxyquinazolin-7-yl)-3-cyano-7-fluorobenzo[b]thiophen-2-yl)carbamate (15.0 mg, 1.0 equiv) in DCM (0.5 mL) was added TFA (1.00 mL, 592 equiv). The reaction was stirred at 25° C. for 1 hour. The mixture was concentrated and purified by prep-HPLC [Phenomenex luna C18 150×25 mm×10 μm; A: water (FA), B: ACN, B %: 24%-54% over 14 min] to afford the title compound (6.82 mg, 53% yield, FA) as white solid; ¹H NMR (400 MHz, METHANOL-d4) δ=8.06 (d, J=1.6 Hz, 1H), 7.21 (dd, J=5.2, 8.4 Hz, 1H), 7.05 (dd, J=8.4, 9.6 Hz, 1H), 5.57-5.34 (m, 1H), 4.61-4.47 (m, 2H), 4.25 (s, 3H), 3.74-3.55 (m, 3H), 3.28-3.20 (m, 1H), 2.62-2.49 (m, 1H), 2.49-2.41 (m, 1H), 2.36-2.27 (m, 1H), 2.24-2.13 (m, 2H), 2.12-2.02 (m, 1H); LCMS (ESI, M+1): m/z=560.1.

### Example 221

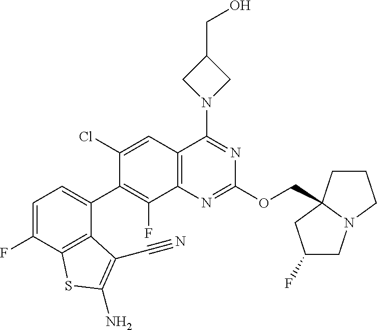 [SMILES: N#Cc1c(N)sc2c(F)ccc(-c3c(Cl)cc4c(N5CC(CO)C5)nc(OC[C@@]56CCCN5C[C@H](F)C6)nc4c3F)c12]

### 2-amino-4-(6-chloro-8-fluoro-2-(((2R,7aS)-2-fluorotetrahydro-1H-pyrrolizin-7a(5H)-yl)methoxy)-4-(3-(hydroxymethyl)azetidin-1-yl)quinazolin-7-yl)-7-fluorobenzo[b]thiophene-3-carbonitrile

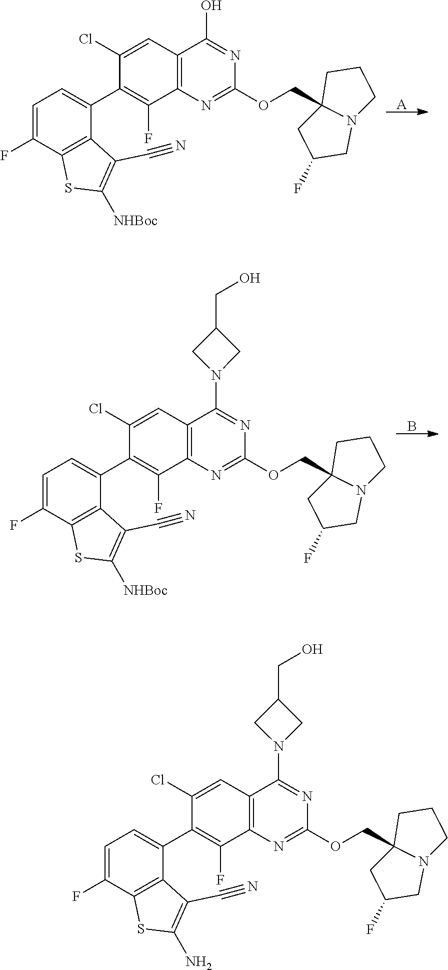

Step A. tert-butyl (4-(6-chloro-8-fluoro-2-(((2R,7aS)-2-fluorotetrahydro-1H-pyrrolizin-7a(5H)-yl)methoxy)-4-(3-(hydroxymethyl)azetidin-1-yl)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (20.0 mg, 1.0 equiv) and PyBOP (24.2 mg, in DMF (0.5 mL) were added DIEA (20.0 mg, 5.0 equiv) and azetidin-3-ylmethanol (19.1 mg, 5.0 equiv, HCl). The reaction was stirred at 25° C. for 1 hour. The mixture was quenched by addition of water (2 mL) at 0° C. and extracted with ethyl acetate (3×3 mL). The combined organic layers were washed with brine (3×3 mL), dried over anhydrous sodium sulfate and concentrated to afford the title compound (22.0 mg, 99% yield) as yellow solid.

Step B. 2-amino-4-(6-chloro-8-fluoro-2-(((2R,7aS)-2-fluorotetrahydro-1H-pyrrolizin-7a(5H)-yl)methoxy)-4-(3-(hydroxymethyl)azetidin-1-yl)quinazolin-7-yl)-7-fluorobenzo[b]thiophene-3-carbonitrile: To a solution of tert-butyl (4-(6-chloro-8-fluoro-2-(((2R,7aS)-2-fluorotetrahydro-1H-pyrrolizin-7a(5H)-yl)methoxy)-4-(3-(hydroxymethyl)azetidin-1-yl)quinazolin-7-yl)-3-cyano-7-fluorobenzo[b]thiophen-2-yl)carbamate (20.0 mg, 1.0 equiv) in DCM (1.0 mL) was added TFA (2.00 mL, 963 equiv) at 0° C. The reaction was stirred at 25° C. for 1 hour. The mixture was concentrated under reduced pressure. The residue was dissolved with MeOH (3 mL), adjusted to pH=7 using NaHCO₃ solid, filtered, concentrated and purified by prep-HPLC [C18 150×30 mm; A: water (FA), B: ACN, B %: 22%-52% over 7 min] to afford the title compound (5.97 mg, 34% yield, FA) as white solid; ¹H NMR (400 MHz, METHANOL-d4) δ=8.53 (s, 1H), 7.85 (d, J=1.2 Hz, 1H), 7.19 (dd, J=5.2, 8.4 Hz, 1H), 7.04 (dd, J=8.4, 9.6 Hz, 1H), 5.56-5.22 (m, 1H), 4.65-4.53 (m, 2H), 4.52-4.24 (m, 4H), 3.81 (d, J=6.0 Hz, 2H), 3.67-3.41 (m, 3H), 3.24-3.12 (m, 1H), 3.11-2.98 (m, 1H), 2.53-2.32 (m, 2H), 2.30-2.18 (m, 1H), 2.17-2.05 (m, 2H), 2.03-1.88 (m, 1H); LCMS (ESI, M+1): m/z=615.2.

### Example 222

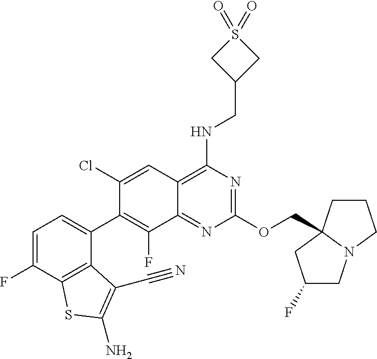 [SMILES: N#Cc1c(N)sc2c(F)ccc(-c3c(Cl)cc4c(NCC5CS(=O)(=O)C5)nc(OC[C@@]56CCCN5C[C@H](F)C6)nc4c3F)c12]

### 2-amino-4-(6-chloro-4-(((1,1-dioxidothietan-3-yl)methyl)amino)-8-fluoro-2-(((2R,7aS)-2-fluorotetrahydro-1H-pyrrolizin-7a(5H)-yl)methoxy)quinazolin-7-yl)-7-fluorobenzo[b]thiophene-3-carbonitrile

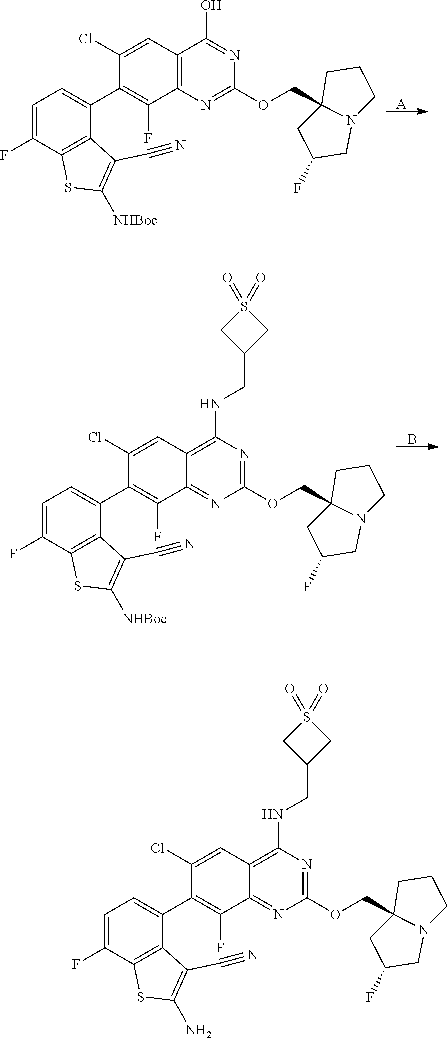

Step A. tert-butyl (4-(6-chloro-4-(((1,1-dioxidothietan-3-yl)methyl)amino)-8-fluoro-2-(((2R,7aS)-2-fluorotetrahydro-1H-pyrrolizin-7a(5H)-yl)methoxy)quinazolin-7-yl)-3-cyano-7-fluorobenzo[b]thiophen-2-yl)carbamate: To a solution of tert-butyl (4-(6-chloro-8-fluoro-2-(((2R,7aS)-2-fluorotetrahydro-1H-pyrrolizin-7a(5H)-yl)methoxy)-4-hydroxyquinazolin-7-yl)-3-cyano-7-fluorobenzo[b]thiophen-2-yl)carbamate (70.0 mg, 1.0 equiv) in DMSO (1 mL) were added PyBOP (84.0 mg, 1.5 equiv) and TEA (88.0 mg, 8.0 equiv). The reaction was stirred at 30° C. for 0.5 hours. 3-(aminomethyl)thietane 1,1-dioxide hydrogen chloride (93 mg, 5.0 equiv) was added into the mixture. The reaction was stirred at 30° C. for 12 hours. The mixture was filtered and purified with reversed phase flash [0.1% FA condition] to afford the title compound (80.0 mg, 72% yield) as yellow solid; LCMS (ESI, M+1): m/z=763.2.

Step B. 2-amino-4-(6-chloro-4-(((1,1-dioxidothietan-3-yl)methyl)amino)-8-fluoro-2-(((2R,7aS)-2-fluorotetrahydro-1H-pyrrolizin-7a(5 h)-yl)methoxy)quinazolin-7-yl)-7-fluorobenzo[b]thiophene-3-carbonitrile: To a solution of tert-butyl (4-(6-chloro-4-(((1,1-dioxidothietan-3-yl)methyl)amino)-8-fluoro-2-(((2R,7aS)-2-fluorotetrahydro-1h-pyrrolizin-7a(5H)-yl)methoxy)quinazolin-7-yl)-3-cyano-7-fluorobenzo[b]thiophen-2-yl)carbamate (70.0 mg, 1.0 equiv) in DCM (0.5 mL) was added TFa (1.59 g, 205 equiv) at 0° C. The reaction was stirred at 20° C. for 0.5 hours. The mixture was added into saturated Na₃HCO₃ aqueous (10 mL) at 0° C. and extracted with EtOAc (4×5 mL). The combined organic layers were dried over anhydrous sodium sulfate, concentrated and purified with prep-HPLC [column: Phenomenex luna C18 150×25 mm×10 μm; mobile phase:water (FA)−ACN; gradient:20%-40% B over 10 minutes] to afford the title compound (5.94 mg, 12% yield, 0.71 HCOOH) as white solid; ¹H NMR (400 MHz, METHANOL-d4) δ=8.11 (s, 1H), 7.20 (dd, J=5.2, 8.4 Hz, 1H), 7.04 (t, J=8.8 Hz, 1H), 5.59-5.36 (m, 1H), 4.68-4.43 (m, 3H), 4.42-4.22 (m, 2H), 4.14-3.90 (m, 4H), 3.86-3.51 (m, 3H), 3.15-2.95 (m, 1H), 2.65-2.29 (m, 3H), 2.27-2.00 (m, 3H); LCMS (ESI, M+1): m/z=663.2.

### Example A: KRas Binding Assay

This Example illustrates that exemplary compounds of the present invention bind to KRas and are capable of displacing a labeled tracer ligand occupying the KRas binding site. KRas^{WT}, KRas^{G12A}, KRas^{G12C}, KRas^{G12D}, KRas^{G12R}, KRas^{G12S}, KRas^{G12V}, KRas^{G13D}, or KRas^{Q61H} was used in the assay.

The ability of a compound to bind to KRas was measured using a TR-FRET displacement assay. Biotinylated KRas (corresponding to amino acids 1-169, produced at Accelegan Inc.) was incubated with custom made Cy5 labelled tracer, terbium streptavidin (Cisbio Inc.) and compound (1% DMSO final) in buffer (50 mM HEPES, pH 7.5, 5 mM MgCl₂, 0.005% Tween-20 and 1 mM DTT). After a 60-minute incubation at room temperature, the reaction was measured using a BMG LABTECH CLARIO star Plus via TR-FRET. 100 percent of control (POC) is determined by using a DMSO control and 0 POC is determined using a concentration of control compound that completely inhibits binding of the tracer to KRas. The POC values were fit to a 4-parameter IC₅₀ equation and the IC₅₀ value reported in Table 1 (selected Examples 172-222).

TABLE 1
Binding to KRas (IC₅₀ nM) by
Exemplary Compounds of Formula (I)

| Example | G12D | G12V | G12R | WT | G12C | G12S |
|---|---|---|---|---|---|---|
| 172 | 34 | 3 | | 11 | <2 | 3 |
| 173 | 661 | 430 | | 751 | 454 | 331 |
| 174 | 285 | 9 | 9 | | | |
| 175 | 313 | 12 | 16 | | | |
| 176 | <2 | <2 | | <2 | <2 | <2 |
| 177 | 107 | 18 | | 40 | 36 | 42 |
| 178 | 92 | 10 | | 17 | 20 | 28 |
| 187 | 2 | <2 | | 2 | <2 | 2 |
| 191 | <2 | <2 | | <2 | <2 | <2 |
| 198 | 9 | 13 | | 7 | 12 | 11 |
| 199 | 10 | 2 | | 6 | 3 | 3 |
| 200 | 3 | 3 | | 2 | 4 | 2 |
| 201 | 3 | 3 | | 3 | 3 | 2 |
| 202 | 6 | 4 | | 7 | 5 | 3 |
| 203 | 18 | 8 | | 12 | 11 | 11 |
| 204 | 3 | 3 | | 2 | 3 | <2 |
| 205 | 3 | 2 | | 3 | <2 | 2 |
| 206 | 14 | 4 | | 6 | 7 | 5 |
| 207 | 2 | <2 | | <2 | <2 | <2 |
| 208 | <2 | 10.372 | | 4 | 7 | 4 |
| 209 | 7 | <2 | | 2 | 4 | 2 |
| 210 | <2 | <2 | | <2 | <2 | <2 |
| 211 | 3 | 2 | | <2 | <2 | <2 |
| 212 | 14 | 11 | | 6 | 6 | 6 |
| 213 | 9 | 4 | | 11 | 8 | 5 |
| 214 | 80 | 15 | | 27 | 14 | 21 |
| 215 | <2 | <2 | | <2 | 4 | <2 |
| 216 | 4 | 4 | | 6 | 6 | 5 |
| 217 | 959 | 75 | | 251 | 184 | 275 |
| 218 | 7 | 3 | | 3 | 3 | 2 |
| 219 | 16 | 4 | | 7 | 9 | 6 |
| 220 | 26 | 7 | | 15 | 13 | 17 |
| 221 | 11 | 3 | | 4 | 3 | 4 |
| 222 | 4 | <2 | | <2 | <2 | <2 |

### Example B

Inhibition of KRas Phosphorylation of ERK (HTRF) by Exemplary Compounds of Formula (I)
Cisbio HTRF Advanced pERK Assay Catalog #64AERPEH
Cell: MKN1, PSN1
   Procedure:
   Day 1: Seed 6,000 cells/well −25 μl/well in 384-well white solid bottom plate; RPM1_10% FBS. Incubate overnight at 37° C./5% C02.
   Day 2: Echo transfer 25 nl of 10 mM compound 10 point dilution at 1:3 (Cf=10 uM) and incubate for 3 hour at 37° C./5% C02.
   Add 8.5 μl/well of 4× Lysis Buffer/25× Blocking reagent (do not dump media) and incubate for 30 min at room temperature on shaker.
   Add conjugate mixture of 4.25 ul/well 1×-pERK-D2 and 1×-pERK-K diluted in Detection Buffer for a total of 8.5 μl/well.
   Incubate for 4 hours at room temperature covered.
   Read HTRF using ClarioStar
Cell: ASPC1, H727, A549, H460, HCT116, H358
   Culture/Assay media: RPMI-1640+10% FBS
   Procedure:

Cell Seeding
1. To harvest cells from flask using 0.05% Trypsin/EDTA solution. Add 10 mL of media to stop trypsinizing. Pipette the cells into a conical bottom 50 mL centrifuge tube and centrifuge 5 min×1000 rpm.
2. Re-suspend the cell pellet in media, take a cell count, and then adjust the cell density using fresh media.
3. Seed 6,000 cells into cell culture plate with 50 μL media. The
4. Incubate cell plate overnight in a 37° C., 5% CO₂ incubator.

Compound Titrations
1. Use Tecan to complete the compound addition. Compounds start from 10 uM top, 3-fold dilution, and 10 doses. The final DMSO concentration is 0.8%. Dispensed 0.2 uM Trametinib as Min control.
2. Incubate cell plate for 3 hrs in the incubator.

Detection with Cisbio pERK HTRF Kit
1. Dilute 1 volume of 4× lysis buffer with 3 volumes of deionized water. Then, add 100× the blocking reagent. Keep lysis buffer on the ice.
2. At the end of the compound treatment, flick-off the media.
3. Add 35 μL of lysis buffer per well using a Multidrop Combi. Then place on a plate agitator shaking at 300 rpm at 4° C. for 40 mins.
4. Make up the HTRF antibody buffer. For each assay plate, mi×50 μL of d2-conjugate antibody with 950 μL of detection buffer. Similarly, mi×50 μL of Cryptate antibody with 950 μL of detection buffer. Then mix the two diluted antibodies together.
5. Dispense 3.4 μL the antibody buffer to wells of an empty assay plate. Seal the plate and centrifuge plate 30 sec×1000 rpm.
6. At the end of the 4° C. lysis, centrifuge the lysate plates 3 mins×1500 rpm.
7. Use the Bravo to transfer 13.6 μL of lysate from cell culture plate to assay plate. Then incubate assay plate for 2 hrs at room temperature.
8. At the end of incubation, read plate on the Envision after centrifuging plate 30 sec×1000 rpm. pERK IC_{50S} were reported in Table 2 and single point % inhibitions at 100 nM were reported in Table 3.

TABLE 2
Inhibition (HTRF IC₅₀ nM) of KRas-mediated
Phosphorylation of ERK by Exemplary Compounds of Formula (I)

| Example | AsPC-1 | H727 | HCT116 | A549 | H460 | H358 | H2009 | PSN1 | MKN1 |
|---|---|---|---|---|---|---|---|---|---|
| 172 | 794 | 111 | 841 | 111 | 182 | 15 | 8 | 4294 | |
| 173 | 1893 | 252 | 2898 | 244 | 511 | 95 | 40 | >10000 | |
| 174 | 4019 | 694 | 2261 | 734 | 968 | 324 | | | |
| 175 | 1281 | 422 | 1639 | 490 | 445 | 185 | | | |
| 176 | 10 | 23 | 135 | 72 | 101 | 8 | | | |
| 179 | 116 | 6808 | 3768 | 3135 | 2317 | 2609 | | | |
| 180 | 1 | 512 | 72 | 139 | 81 | 54 | 40 | 1373 | 10 |
| 181 | 950 | 8498 | 8029 | >10000 | 9665 | 4563 | | 5259 | |
| 182 | 4 | 1386 | 114 | 377 | 207 | 136 | | 1817 | |
| 183 | 254 | >10000 | 4795 | >10000 | 8089 | 6740 | | 6153 | |
| 184 | 1 | 610 | 39 | 160 | 82 | 40 | | 701 | |
| 185 | 2 | 3 | 17 | 12 | 9 | 1 | | 1802 | |
| 186 | 162 | 202 | 670 | 382 | 341 | 98 | | >10000 | |
| 187 | 41 | 148 | 176 | 106 | 78 | 12 | | 1063 | |
| 188 | 3 | 10 | 18 | 14 | 13 | 2 | | 544 | |
| 189 | 472 | 882 | 2467 | 3347 | 1527 | 327 | | 7466 | |
| 190 | 627 | 870 | 596 | 525 | 347 | 330 | | 5566 | |
| 191 | 8 | 12 | 11 | 11 | 10 | 2 | | 798 | |
| 192 | 3 | 5 | 12 | 8 | 4 | 1 | 1 | 260 | 1 |
| 193 | 414 | 686 | | | | | | | |
| 194 | 192 | 342 | 896 | 605 | 427 | 128 | | | |
| 195 | 0 | 2 | 6 | 2 | 2 | 1 | | 115 | |
| 196 | 531 | 2815 | | 2425 | | 472 | | | |
| 197 | 2 | 12 | | 10 | | 3 | | | |
| 198 | 253 | 717 | 275 | 326 | 170 | 125 | | | |
| 199 | 4205 | 4090 | 889 | 1179 | 462 | 232 | | 3856 | |
| 200 | 2738 | 4728 | 4559 | >10000 | 2760 | 1683 | | | |
| 201 | 4693 | >10000 | >10000 | >10000 | 5338 | 1779 | | >10000 | |
| 202 | 200 | 104 | 498 | 200 | 145 | 63 | 24 | 3347 | |
| 203 | 501 | 226 | 661 | 307 | 306 | 82 | | 4792 | |
| 204 | 16 | 18 | 25 | 34 | 23 | 6 | | 1175 | |
| 205 | 1744 | 554 | 985 | 574 | 690 | 71 | | 7121 | |
| 206 | 246 | 55 | 367 | 244 | 173 | 60 | | 4892 | |
| 207 | 3 | 11 | 42 | 22 | 34 | 3 | | 811 | |
| 208 | 4 | 3279 | 90 | 418 | 260 | 167 | | 2392 | |
| 209 | 93 | 27 | 83 | 68 | 47 | 9 | 15 | 1687 | |
| 210 | 21 | 22 | 54 | 38 | 25 | 10 | 5 | 1188 | |
| 211 | 22 | 12 | 18 | 15 | 13 | 3 | | 1274 | |
| 212 | 230 | 357 | | 76 | | 22 | | | |
| 213 | 761 | 767 | | 398 | | 160 | | | |
| 214 | >10000 | >10000 | >10000 | >10000 | 5055 | 982 | 4694 | >10000 | 739 |
| 215 | 25 | 35 | 392 | 393 | 223 | 21 | 34 | 3817 | 13 |
| 216 | 175 | 131 | 346 | 325 | 118 | 26 | 18 | 2435 | 12 |
| 218 | 1652 | 429 | 566 | 720 | 365 | 57 | 80 | 5112 | |
| 219 | 2901 | 1069 | 2254 | 3343 | 1120 | 370 | 508 | >10000 | 266 |
| 220 | 202 | 123 | 591 | 261 | 134 | 63 | 49 | 5866 | 45 |
| 221 | 962 | 1236 | 573 | 635 | 394 | 154 | 145 | 6035 | 86 |
| 222 | 2560 | 1439 | 1109 | 1641 | 911 | 207 | 273 | >10000 | 84 |

TABLE 3
Inhibition (HTRF % at 100 nM) of KRas-
mediated Phosphorylation of ERK by
Exemplary Compounds of Formula (I)

| Ex. No. | AsPC-1 | H727 | H460 | H358 | A549 | HCT116 |
|---|---|---|---|---|---|---|
| 11 | 91 | 49 | | | | |
| 28 | | | 8 | | | 9 |
| 36 | | | 25 | | | −22 |
| 37 | | | 28 | | | 0 |
| 38 | | | | 16 | 9 | |
| 39 | | | 11 | | | 3 |
| 42 | | | 59 | 71 | 39 | 30 |
| 44 | | | | 3 | −9 | |
| 45 | | | 94 | | | 86 |
| 48 | | | 75 | | | 50 |
| 49 | | | 23 | | | 13 |
| 50 | | | 38 | 3 | −17 | −3 |
| 51 | | | | 67 | 6 | |
| 52 | | | | 6 | 33 | |
| 53 | | | | 40 | 27 | |
| 54 | | | | 100 | 61 | |
| 55 | | | | 2 | −13 | |
| 56 | | | 86 | 87 | 47 | 51 |
| 58 | | | 58 | | | 12 |
| 59 | | | 36 | | | 23 |
| 61 | | | 36 | | | 9 |
| 62 | | | 98 | 96 | 72 | 92 |
| 63 | | | 82 | | | 61 |
| 64 | | | 3 | | | 14 |
| 65 | | | 1 | | | 16 |
| 66 | | | 97 | | | 97 |
| 67 | | | 95 | 94 | 81 | 73 |
| 68 | | | 14 | | | 8 |
| 69 | | | | 101 | 84 | |
| 70 | | | | 24 | 6 | |
| 71 | 90 | 65 | 87 | 94 | 82 | 38 |
| 72 | | | 87 | 98 | 76 | 69 |
| 73 | 97 | 71 | | 97 | 71 | 48 |
| 74 | 99 | 78 | | 98 | 72 | 38 |
| 75 | 95 | 73 | | 98 | 79 | 29 |
| 76 | 90 | 43 | 31 | 90 | 42 | 16 |
| 77 | 87 | 66 | 86 | 96 | 82 | 45 |
| 78 | 98 | 75 | 81 | 96 | 94 | 75 |
| 79 | | | 85 | 95 | 60 | 64 |
| 80 | | | 89 | 95 | 83 | 71 |
| 81 | | | 26 | 98 | 62 | 32 |
| 82 | 72 | 34 | 63 | 80 | 33 | 24 |
| 85 | | | 16 | −7 | 21 | −12 |
| 86 | | | −1 | −14 | 0 | −12 |
| 87 | | | −8 | 30 | 16 | −5 |

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth, and as follows in the scope of the appended claims.

What is claimed is:

1. A compound of Formula (I): 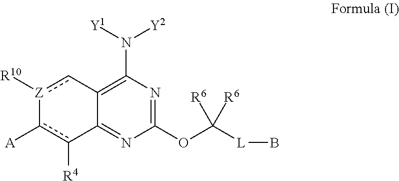
or a pharmaceutically acceptable salt thereof, wherein:
A is aryl or heteroaryl, wherein the aryl or the heteroaryl is optionally substituted with 1-4 R¹;
B is: 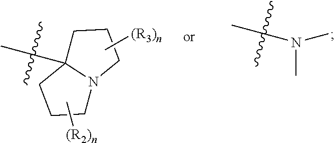
Y¹ is hydrogen, hydroxy, halogen, L-SO₂—NH₂, L-OH, C1-C4 alkyl, L-C3-C6 cycloalkyl optionally substituted with 1-4 R⁹, L-heteroaryl optionally substituted with 1-4 R⁸, L-aryl optionally substituted with 1-4 R⁸, L-C(O)—NH₂, and L-heterocycle optionally substituted with 1-2 oxo (═O) or oxo-containing substituent, and optionally further substituted with 1-2 R⁸;
Y² is hydrogen or C1-C4 alkyl;
or Y¹ and Y² join to form: 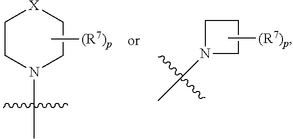
where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—;
each R¹ is independently halogen, cyano, hydroxy, C1-C4 alkyl, —S—C1-C3 alkyl, C2-C4 alkenyl, C2-C4 alkynyl, C2-C4 hydroxyalkynyl, C1-C3 cyanoalkyl, triazolyl, C1-C3 haloalkyl, —O—C1-C3 haloalkyl, —S—C1-C3 haloalkyl, C1-C3 alkoxy, hydroxyC1-C3 alkyl, —CH₂C(═O)N(R⁵)₂, —C3-C4 alkynyl(NR⁵)₂, —N(R⁵)₂, deuteroC2-C4 alkynyl, (C1-C3 alkoxy)haloC1-C3 alkyl-, or C3-C6 cycloalkyl wherein said C3-C6 cycloalkyl is optionally substituted with halogen or C1-C3 alkyl;
each R² is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —OC(O)N(R⁵)₂, —CO₂R⁵, or —CO₂N(R⁵)₂;
each R³ is independently hydrogen, deuterium, hydroxy, halogen, C1-C3 alkyl, ═CH₂, ═CH(halogen), ═C(halogen)₂, C1-C3 cyanoalkyl, C1-C3 hydroxyalkyl, HC(═O)—, —COC(O)N(R⁵)₂, —CO₂R⁵, or —CO₂N(R⁵)₂;
R⁴ is hydrogen, halogen or C1-C3 alkyl;
each R⁵ is independently hydrogen or C1-C3 alkyl;
each R⁶ is independently hydrogen, hydroxy, C1-C4 hydroxyalkyl or heteroaryl;
each R⁷ is independently hydrogen, C1-C3 alkyl, hydroxy, halogen, C1-C3 haloalkyl, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —CN, aryl, —CH₂—S(O)₂NH₂, or heteroaryl optionally independently substituted with 1-2 C1-C3 alkyl, —CN or C(O)NH₂,
two R⁷ on the same atom optionally join to form a spirocyclic ring selected from C3-C6 cycloalkyl and heterocycle, where said spirocyclic ring is optionally substituted with 1-4 substituents independently selected from oxo (═O), halogen, hydroxy, C1-C3 alkyl and —O—(C1-C3 alkyl),
two R⁷ on adjacent atoms optionally join to form a bond or a fused ring selected from C3-C6 cycloalkyl optionally substituted with 1-4 R⁸, heteroaryl optionally substituted with 1-4 R⁸, aryl optionally substituted with 1-4 R⁸, and heterocycle optionally substituted with 1-4 R⁸, and
two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;
each R⁸ is independently C1-C3 alkyl, hydroxy, halogen, —NH₂, —NH(C1-C3 alkyl), —N(C1-C3 alkyl)₂, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂, —C(O)-pyrrolidine or —CN;
each R⁹ is independently C1-C3 alkyl, hydroxy, halogen, oxo (═O), —O—(C1-C3 alkyl), -(C1-C3 alkyl)-OH, —C(O)OH, —C(O)O(C1-C3 alkyl), —C(O)NH₂, —C(O)NH(C1-C3 alkyl), —C(O)N(C1-C3 alkyl)₂ or —CN;
R¹⁰ is absent, hydrogen, deuterium, hydroxy, halogen, C1-C3 alkyl, deuterated C1-C3 alkyl, C2-C3 alkenyl, deuterated C2-C3 alkenyl or C3-C6 cycloalkyl;
L is a bond, —C1-C4 alkyl-, —NH—, —N(C1-C3 alkyl)- or cyclopropyl-CH₂—;
Z is C or O, wherein if Z is C the 6-membered ring that includes Z is aromatic, and wherein if Z is O the 6-membered ring that includes Z is an oxane;
each n is 0-3;
o is 1-6; and
p is 1-8.

2. The compound or salt of claim 1, wherein:
A is aryl, optionally substituted with 1-4 R¹; and
Y¹ and Y² join to form: 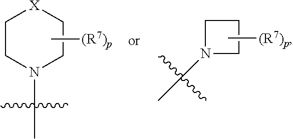
where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—.

3. The compound or salt of claim 1, wherein:
A is heteroaryl, optionally substituted with 1-4 R¹; and
Y¹ and Y² join to form: 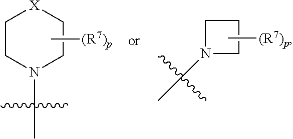
where X is selected from: a bond, —S—, —O—, —N<, —CH₂—N<, —CH₂—CH₂—N<, —CH—, —CH₂—CH₂—, —CH₂—CH₂—CH₂—, —O—CH₂— and —S—CH₂—.

4. The compound or salt of claim 1, wherein B is:

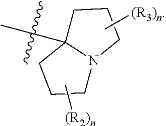

5. The compound or salt of claim 1, wherein -L-B is:

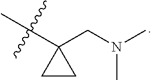 [SMILES: *C1(CN(C)C)CC1]

6. The compound or salt of claim 1, wherein A is naphthyl.

7. The compound or salt of claim 1, wherein A is indazolyl or benzothiophenyl.

8. The compound or salt of claim 1, wherein at least one R¹ is C1-C4 alkyl.

9. The compound or salt of claim 1, wherein at least one R¹ is fluorine.

10. The compound or salt of claim 1, wherein at least one R¹ is hydroxy.

11. The compound or salt of claim 1, wherein at least one R² is fluorine.

12. The compound or salt of claim 1, wherein at least one R³ is halogen.

13. The compound or salt of claim 12, wherein said halogen is fluorine.

14. The compound or salt of claim 1, wherein at least one of R² and R³ is independently selected from the group consisting of ═CH₂, ═CHF, ═CF₂,

15. The compound or salt of claim 1, wherein R⁴ is halogen.

16. The compound or salt of claim 15, wherein said halogen is fluorine.

17. The compound or salt of claim 1, wherein one or both R⁶ are C1-C4 alkyl.

18. The compound or salt of claim 1, wherein one or both R⁶ are hydrogen.

19. The compound or salt of claim 1, wherein two R⁷ on the same atom join to form a spirocyclic ring selected from C3-C6 cycloalkyl and heterocycle, where said spirocyclic ring is optionally substituted with one or more substituents selected from oxo (═O), halogen, hydroxy, C1-C3 alkyl and —O—(C1-C3 alkyl).

20. The compound or salt of claim 1, wherein two R⁷ on adjacent atoms join to form a bond or a fused ring selected from C3-C6 cycloalkyl optionally substituted with 1-4 R⁸; heteroaryl optionally substituted with 1-4 R⁸; aryl optionally substituted with 1-4 R⁸, and heterocycle optionally substituted with 1-4 R⁸.

21. The compound or salt of claim 1, two R⁷ on non-adjacent atoms optionally join to form a bridge comprising 1-3 members selected from (i) —CH₂— optionally substituted with 1-2 substituents selected from hydroxy, cyano, -halogen, C1-C4 alkyl and NH₂, (ii) up to one —O—, (iii) up to one —S— and (iv) up to one —NH—;

22. The compound or salt of claim 20, wherein at least one R⁸ is C1-C4 alkyl.

23. The compound or salt of claim 20, wherein at least one R⁸ is hydroxy or C1-C3 alkyl-hydroxy.

24. The compound or salt of claim 20, wherein one or two R⁸ are oxo (═O).

25. The compound or salt of claim 1, wherein Y¹ and Y² join to form piperidine, azepane, azocane, thiazepine, diazepane, oxazepane, azetidine, pyrrolidine, piperazine bound to a fused ring via nitrogen or thiomorpholine.

26. A compound selected from: 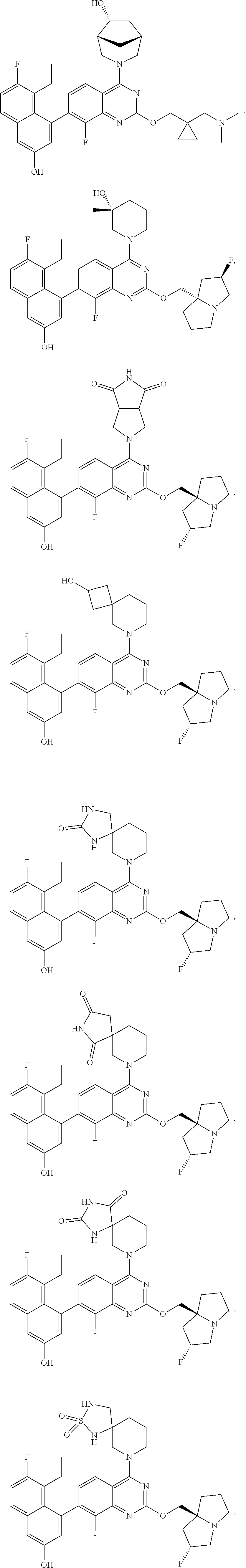 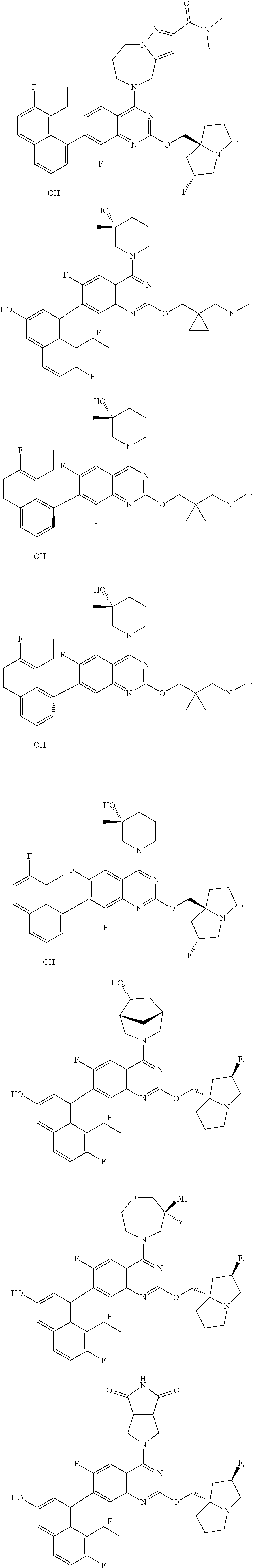 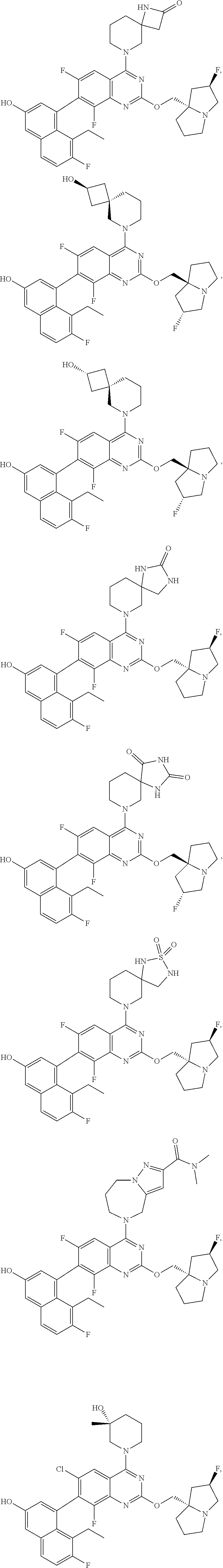 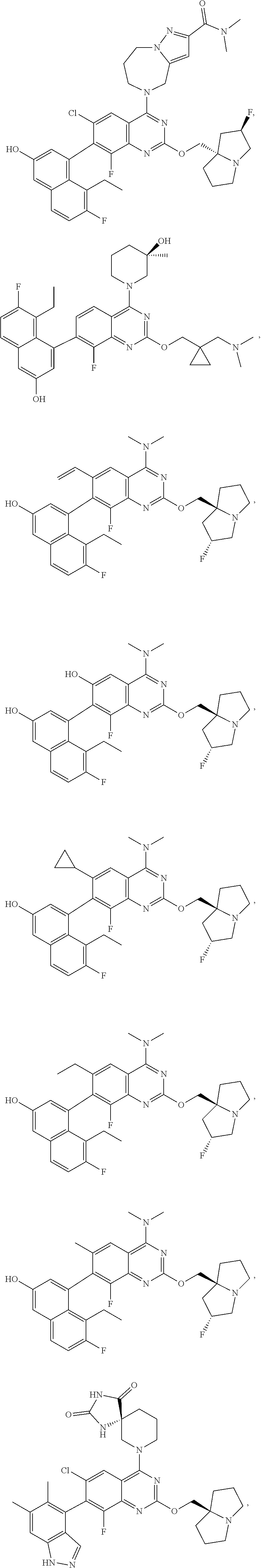 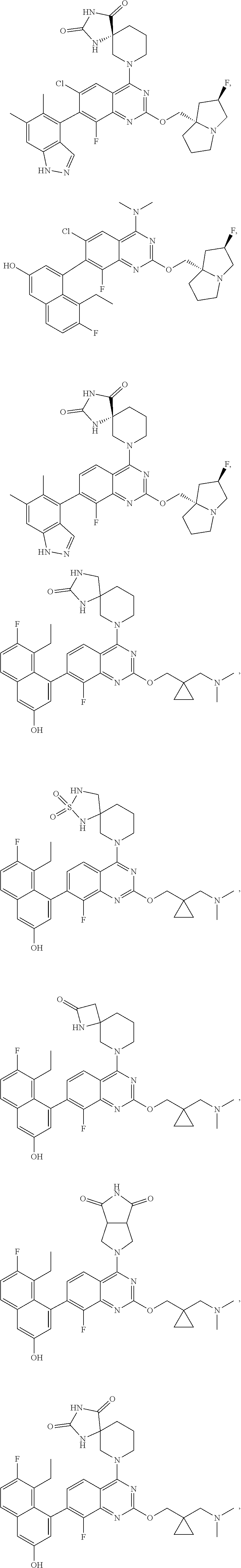 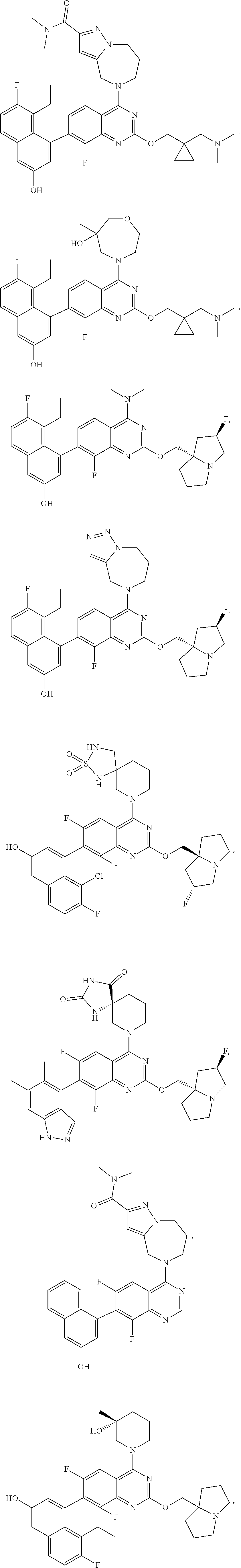 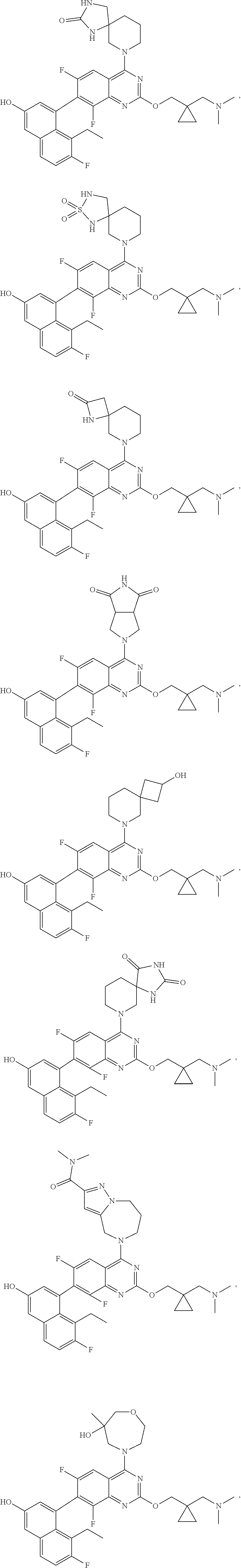 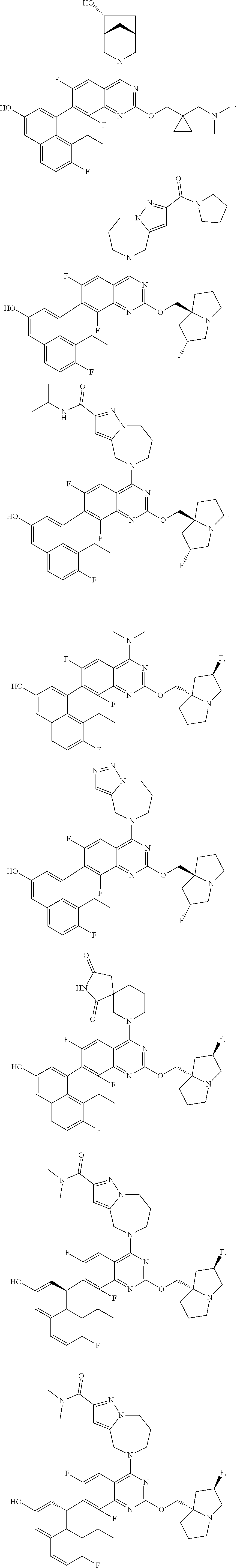 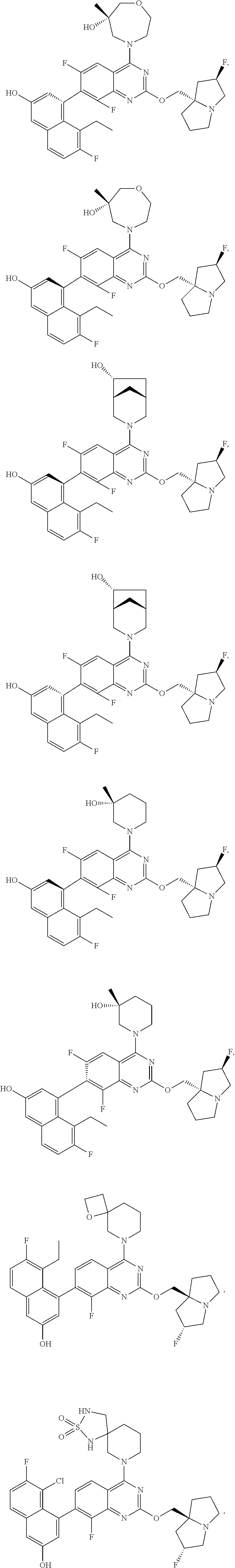 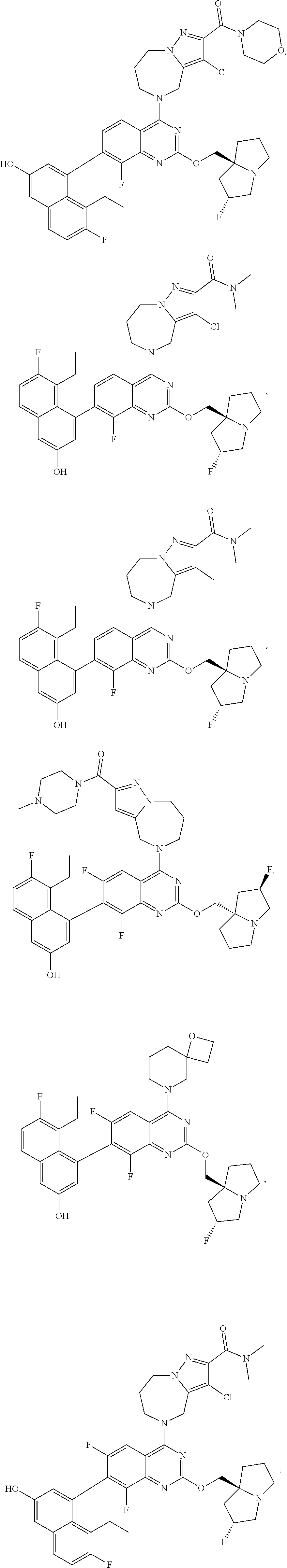 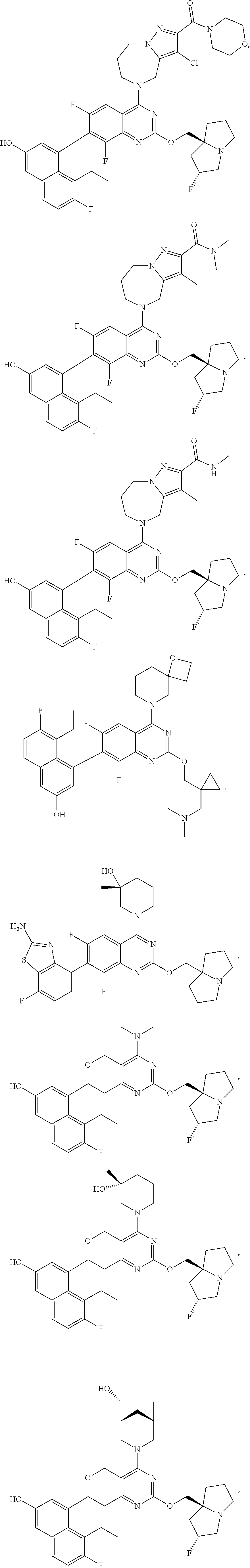 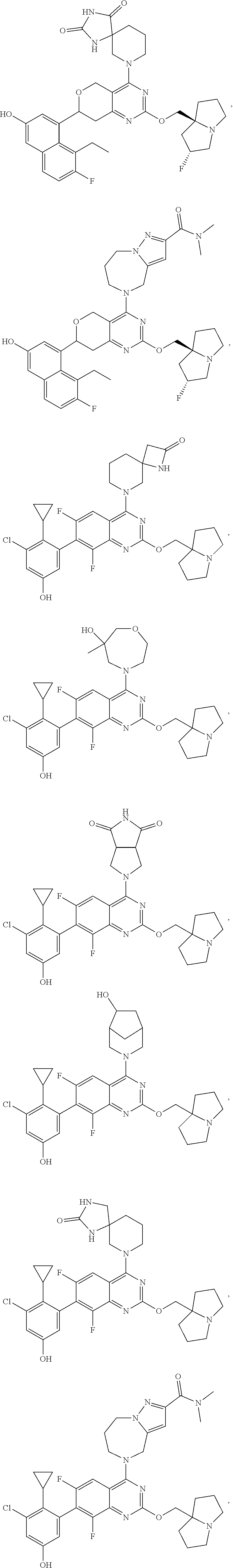 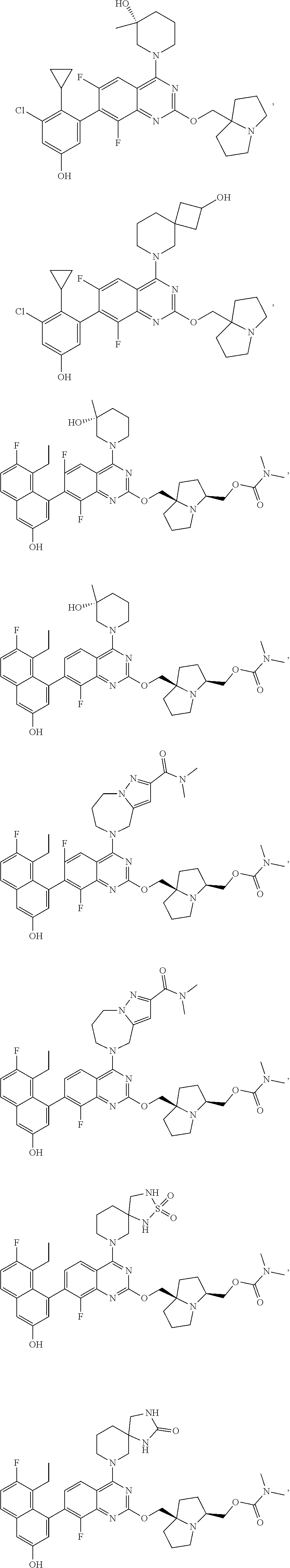 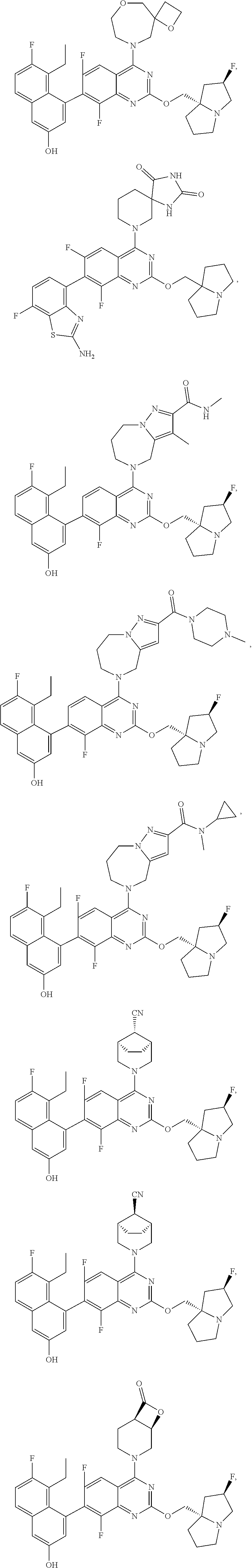 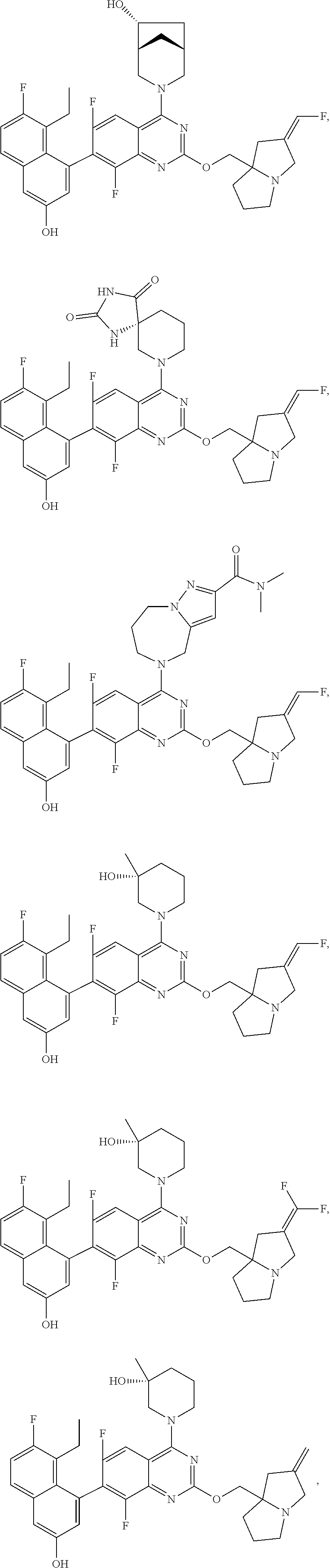 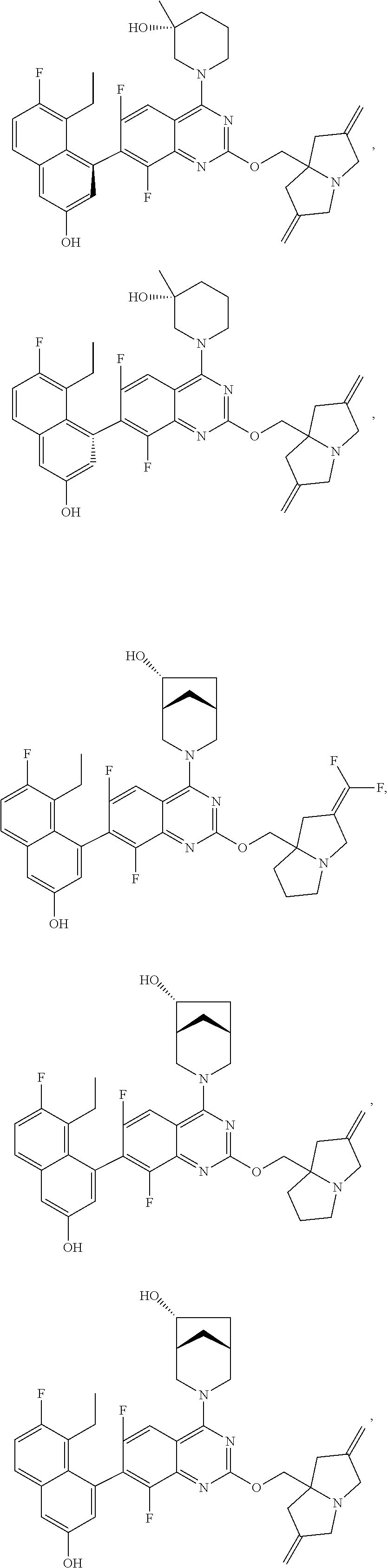 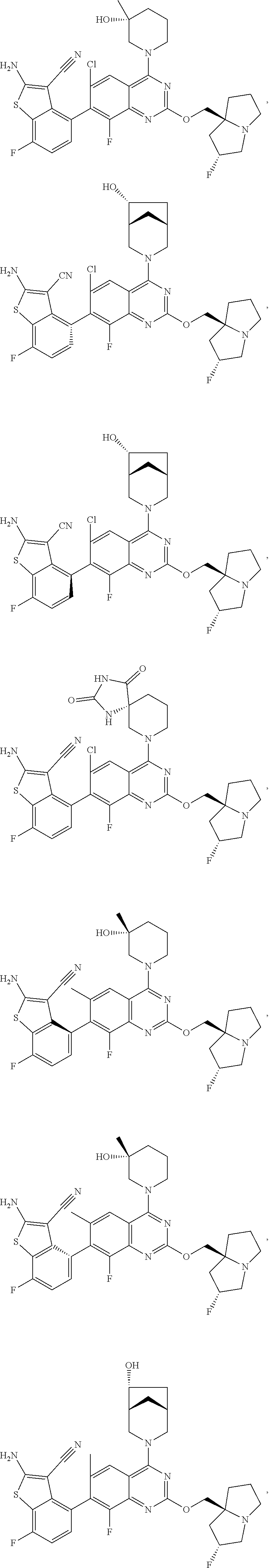 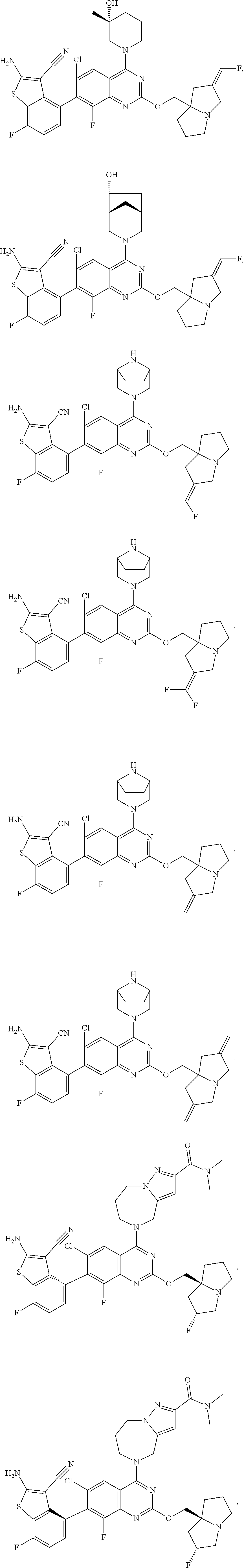 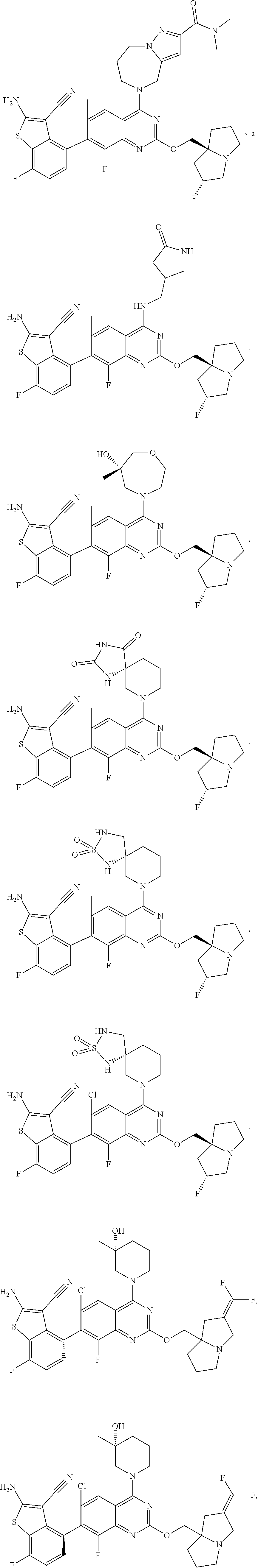 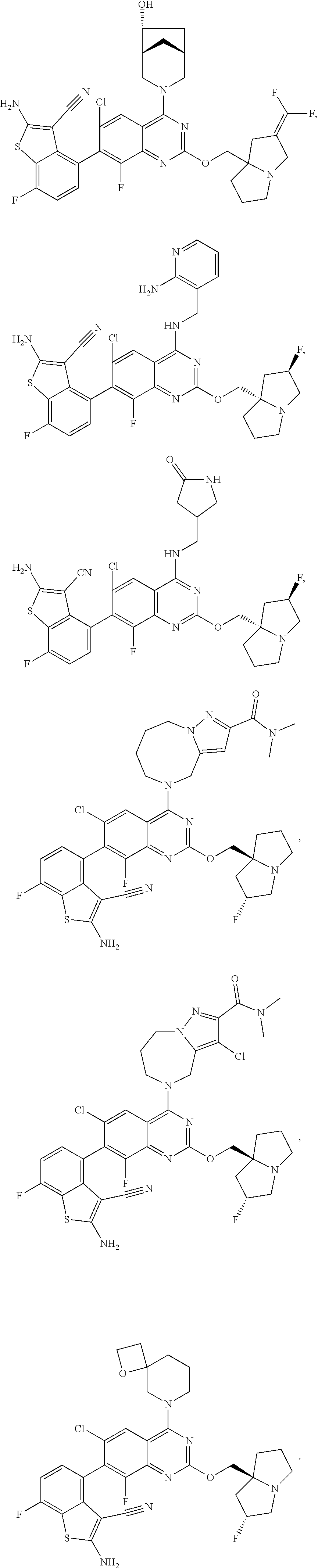 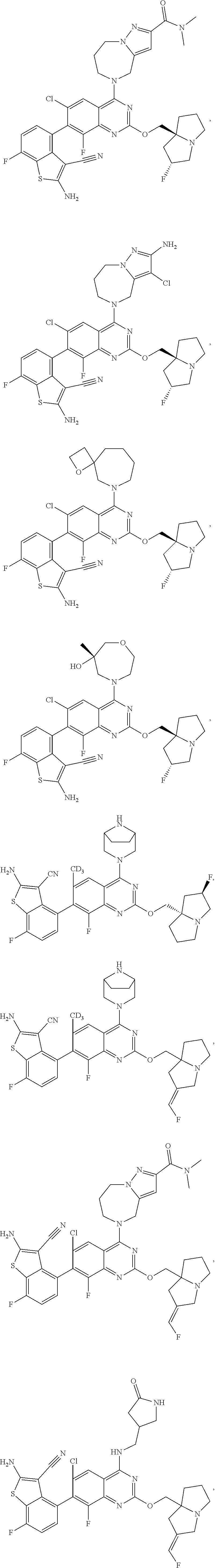 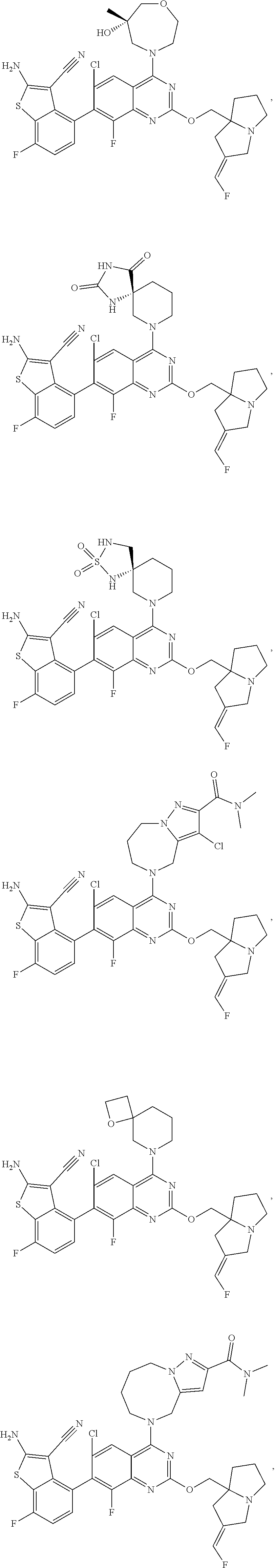 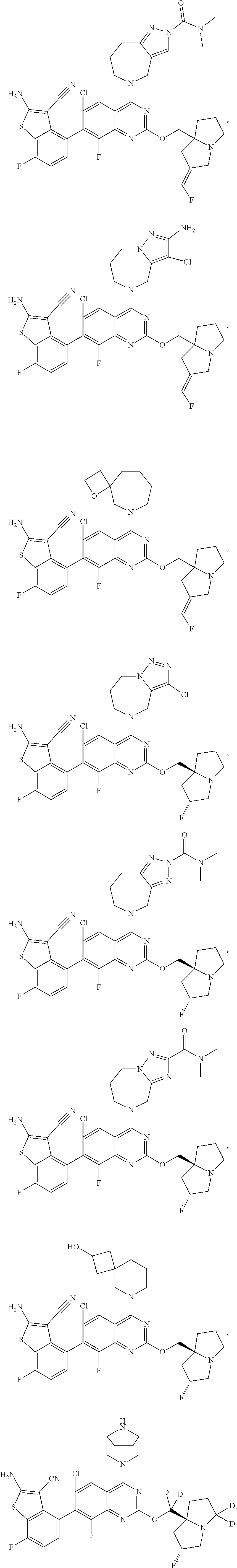 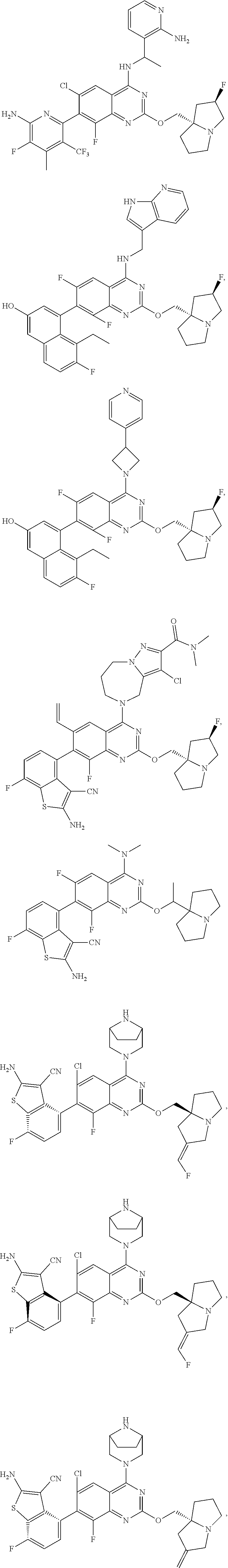 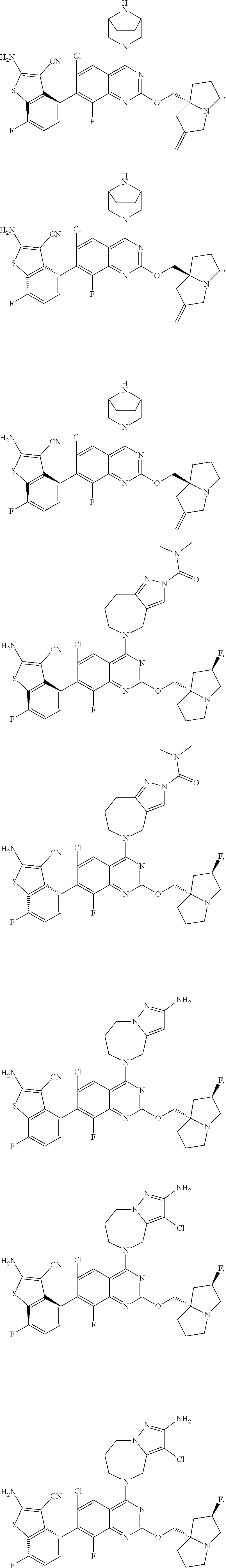 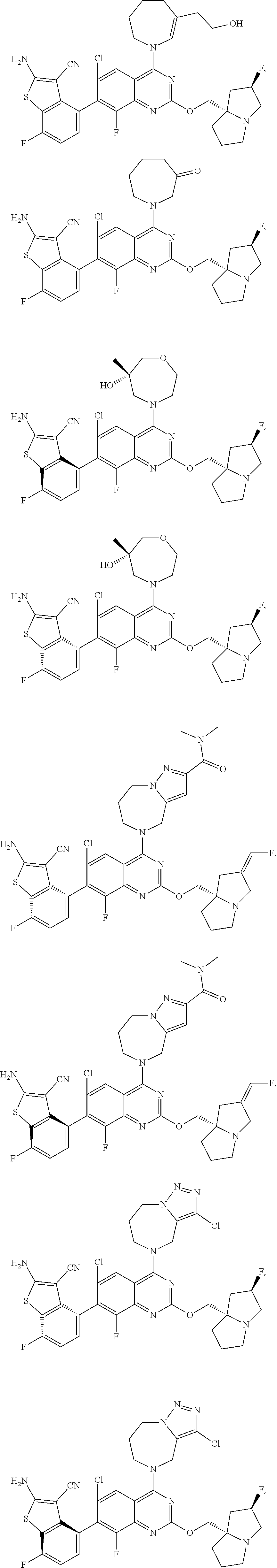 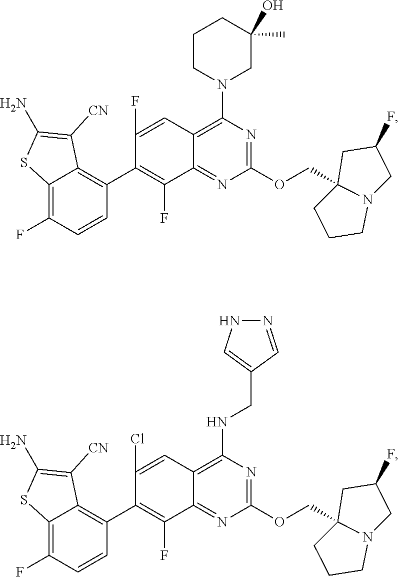 [SMILES: C[C@@]1(O)CCCN(c2nc(OC[C@@]34CCCN3C[C@H](F)C4)nc3c(F)c(-c4ccc(F)c5sc(N)c(C#N)c45)c(F)cc23)C1 ; N#Cc1c(N)sc2c(F)ccc(-c3c(Cl)cc4c(NCc5cn[nH]c5)nc(OC[C@@]56CCCN5C[C@H](F)C6)nc4c3F)c12] 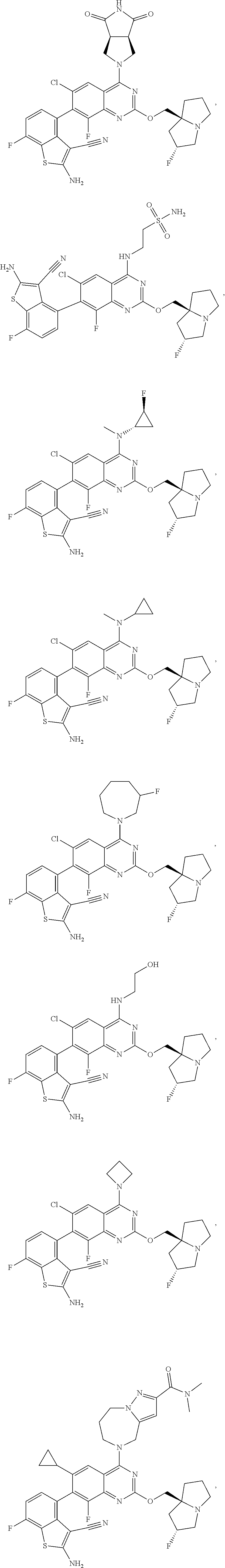 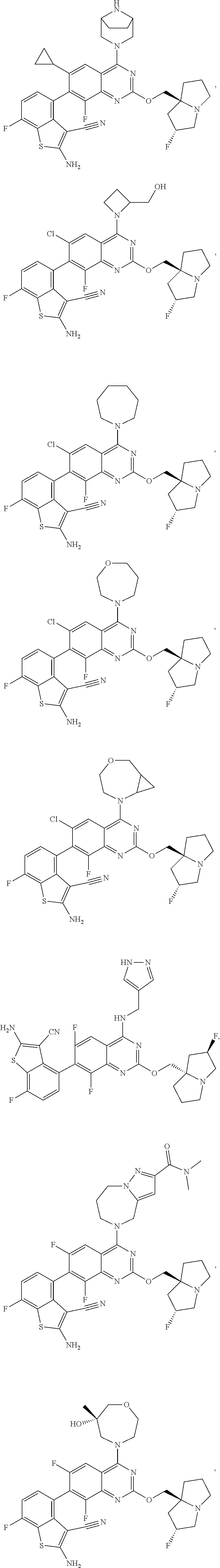 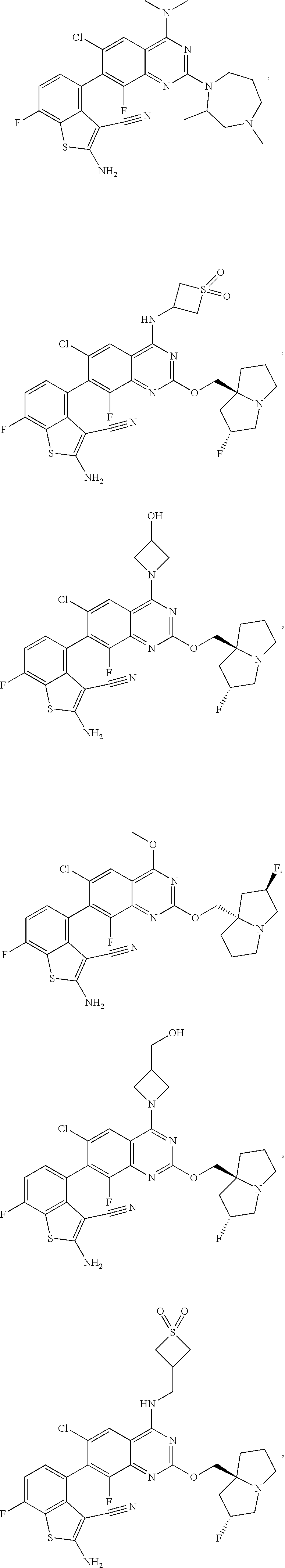
and pharmaceutically acceptable salts thereof.

27. A pharmaceutical composition, comprising a therapeutically effective amount of a compound of claim 1 or a pharmaceutically acceptable salt thereof, and a pharmaceutically acceptable excipient.

28. A method for inhibiting the wild type KRas, KRas G12A, KRas G12C, KRas G12D, KRas G12R, KRas G12S, KRas G12V, KRas G13D or KRas Q61H activity in a cell, comprising contacting the cell in which inhibition of KRas activity is desired with an effective amount of a compound of according to of claim 1 or a pharmaceutically acceptable salt thereof.

29. A method for treating cancer comprising administering to a patient having cancer a therapeutically effective amount of a compound according to claim 1 or a pharmaceutically acceptable salt thereof.

30. The method of claim 29, wherein the therapeutically effective amount of the compound is between about 0.01 to 100 mg/kg per day.

31. The method of claim 30, wherein the therapeutically effective amount of the compound is between about 0.1 to 50 mg/kg per day.

32. The method of claim 29, wherein the cancer is selected from the group consisting of Cardiac: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Kaposi's sarcoma, leiomyoma, hemangioma, lipoma, neurofibroma, fibroma), large bowel (adenocarcinoma, tubular adenoma, villous adenoma, hamartoma, leiomyoma); Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Biliary tract: gall bladder carcinoma, ampullary carcinoma, cholangiocarcinoma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial′carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma); Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma); Skin: malignant melanoma, basal cell carcinoma, squamous cell carcinoma, Kaposi's sarcoma, moles dysplastic nevi, lipoma, angioma, dermatofibroma, keloids, psoriasis; and Adrenal glands: neuroblastoma.

33. The method of claim 32, wherein the cancer is a KRas G12 A-associated cancer.

34. The method of claim 32, wherein the cancer is a KRas G12 C-associated cancer.

35. The method of claim 32, wherein the cancer is a KRas G12 D-associated cancer.

36. The method of claim 32, wherein the cancer is a KRas G12 R-associated cancer.

37. The method of claim 32, wherein the cancer is a KRas G12 S-associated cancer.

38. The method of claim 32, wherein the cancer is a KRas G12 V-associated cancer.

39. The method of claim 32, wherein the cancer is a KRas G13D-associated cancer.

40. The method of claim 32, wherein the cancer is a KRas Q61H-associated cancer.

41. The method of claim 32, wherein the cancer is a KRas G12 A-associated cancer.

42. The method of claim 32, wherein the cancer is associated with at least one of wild type KRas, KRas G12 A, KRas G12 C, KRas G12 D, KRas G12 R, KRas G12 S, KRas G12 V, KRas G13D or KRas Q61H.

43. The method of claim 32, wherein the cancer is non-small cell lung cancer, small cell lung cancer, colorectal cancer, rectal cancer or pancreatic cancer.

44. A method for treating cancer in a patient in need thereof, the method comprising (a) determining that the cancer is associated with wild type KRas or a KRas G12 A, KRas G12 C, KRas G12 D, KRas G12 R, KRas G12 S, KRas G12 V, KRas G13D or KRas Q61H mutation; and (b) administering to the patient a therapeutically effective amount of a compound according to claim 1 or a pharmaceutically acceptable salt thereof.

45. The method of claim 29, wherein the administering is done via a route selected from the group consisting of parenteral, intraperitoneal, intradermal, intracardiac, intraventricular, intracranial, intracerebrospinal, intrasynovial, intrathecal administration, intramuscular injection, intravitreous injection, intravenous injection, intra-arterial injection, oral, buccal, sublingual, transdermal, topical, intratracheal, intrarectal, subcutaneous, and topical administration.

46. The method of claim 45, wherein the administration route is oral.

47. The method of claim 45, wherein the administration is intravenous injection.

48. The method of claim 45, wherein the administration route is intramuscular injection.

49. The method of claim 45, wherein the administration route utilizes a delivery device.

50. The method of claim 45, wherein administration is done in a hospital setting.
